(19) United States
(12) Patent Application Publication
(10) Pub. No.: US 2009/0010908 A1
(43) Pub. Date: Jan. 8, 2009

(54) Materials and Methods for Diagnosis and Treatment of Chronic Fatigue Syndrome

(76) Inventors: John Gow, Glasgow (GB); Abhijit Chaudhuri, Glasgow (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/815,290

(22) PCT Filed: Feb. 1, 2006
(86) PCT No.: PCT/GB2006/000332

§ 371 Date: Jul. 16, 2008

(30) Foreign Application Priority Data
Feb. 1, 2005 (GB) 0502042.5

(51) Int. Cl. A61K 31/122; C40B 50/00; C40B 50/02; C40B 50/06; A61K 31/573; C12Q 1/68; A61K 31/415; C40B 40/00; A61K 31/65

(52) U.S. Cl. 424/94.1; 506/23; 506/24; 506/26; 506/13; 514/154; 514/406; 514/152; 514/179; 435/6

(57) ABSTRACT

The invention relates to materials and methods for diagnosis and treatment of chronic fatigue syndrome/myalgic encephalitis. A number of genes are identified which are expressed at abnormal levels in patients affected by CFS/ME as compared to normal healthy individuals. These genes include those encoding defensin α1, haemoglobin γ, CXCR4, tubulin beta 1, serine/threonine kinase 17B, HLA DRss4 and prostaglandin D2 synthase. The genes identified provide objective disease markers that may be used in diagnostic tests to support the diagnosis of CFS/ME or for monitoring the effectiveness of therapy. They also provide a rational basis for classifying CFS/ME patients according to the biochemical lesion underlying their symptoms and enable provision of appropriate targeted therapies.

### FIELD OF THE INVENTION

The invention relates to chronic fatigue syndrome/myalgic encephalitis, and in particular to materials and methods for its diagnosis and treatment.

### BACKGROUND TO THE INVENTION

Chronic fatigue syndrome (CFS), also known as Myalgic Encephalomyelitis (ME), is an acquired disorder with long term disability¹. The illness affects both children and adults and is characterised by persistent or relapsing fatigue of sufficient severity to interfere with normal function. In addition, patients experience impairment in short term memory and concentration, muscle pain and prolonged post-exertional malaise². The estimated overall prevalence of CFS/ME in the community is approximately 0.2-0.4% in adults and 0.07% in adolescents and children³.

The pathogenesis of CFS/ME is unknown. Diagnosis of CFS/ME according to the internationally accepted definition (modified CDC criteria) is essentially based on exclusion of known medical and psychiatric diseases². As yet, there is no specific or sensitive diagnostic test that positively establishes and/or supports the clinical diagnosis of CFS/ME.

Nevertheless, in 2002, following the publication of a UK Government Working Party report, Professor Sir Liam Donaldson confirmed that CFS/ME is a debilitating and distressing condition affecting many people which should be classed alongside other diseases such as multiple sclerosis and motor neurone disease”.

A significant proportion of patients report antecedent history of community acquired viral or bacterial infections^{4,5,6}. In addition, it is clear that patients with CFS/ME have a hypoactive hypothalamic-pituitary-adrenal (HPA) axis with altered neuroendocrine regulation affecting neurotransmitters such as monoamines (norepinephrine, serotonin and dopamine) and acetylcholine in the central nervous system⁴. Recent studies have demonstrated that viruses can affect neurotransmitter functions^{7,8}.

At the cellular level, fatigue has been linked with alterations in the cell membrane ion-channel traffic and ATPase system⁹. ATPases are also linked with neurotransmitter release (e.g. dopamine)¹⁰ and cellular energy metabolism via creatine phosphatase. Increased ATPase activity has been reported in muscle biopsies from patients with CFS^{11,12}. Previous work^{11,13} has raised the possibility that patients with CFS may have an ion channel dysfunction. This dysfunction might be induced by changes in the ion channel function, neurotransmitters involved in “gating” the channel or by a shift in the balance of the cellular “energy charge”, i.e. the ratio between ATP and ADP that is normally a function of the ATPase activity.

One previous study⁴³ has identified a number of genes differentially expressed between PBMCs from CFS/ME patients and healthy controls. A number of the genes identified are implicated in various aspects of immunological function, from which the authors concluded that some kind of immunological dysfunction may be involved in pathogenesis of CFS/ME. The authors were unable to relate the observed expression patterns to any functional model of disease etiology or pathology, and did not suggest that any of the genes which they identified could serve as useful biomarkers for CFS/ME. Their conclusions are consistent with previous studies which have suggested immunological abnormality in CFS/ME (Refs. 44 to 48) but the precise mechanism of immunological dysfunction in CFS/ME has not been established.

### SUMMARY OF THE INVENTION

The present inventors have identified a number of genes which are expressed at abnormal levels in patients affected by CFS/ME as compared to normal healthy individuals. In contrast to the earlier studies described above, the present inventors have been able to use the expression patterns of these genes to establish functional models of various aspects of the pathology of CFS/ME, which explain many of the symptoms observed in affected individuals. These provide a rational basis for classifying CFS/ME patients according to the biochemical lesion underlying their symptoms and enable appropriate targeted therapies to be provided for the first time.

The genes identified in the present invention provide objective disease markers that may be used in diagnostic tests to support the diagnosis of CFS/ME or in other applications. For example, the tests may enable deselection of inappropriately diagnosed patients who have an alternative diagnosis for their fatigue symptoms. The tests may also be used to classify patients according to the particular biochemical basis for their symptoms. In turn, this may enable a clinician to identify therapies which would be appropriate and rule out those which would not. The tests may also be applied as an outcome measure for interventional trials in CFS/ME, not least because no specific treatment is currently considered to be effective in the patient population defined by the clinical criteria. Further, the tests may offer support to CFS/ME patients for their claims for disability and insurance. Finally, depending on the specificity and sensitivity of the test, one may apply it to screen patients with symptoms of chronic fatigue to identify or exclude CFS/ME. Given the fact that chronic fatigue as a symptom is ten times commoner in the population (2%) than CFS/ME, an estimated size of the market for a screening test is at least 10 million in the UK alone.

Thus the present invention provides a method for investigating whether a test subject is affected by chronic fatigue syndrome/myalgic encephalomyelitis (CFS/ME), the method comprising providing a biological sample from the subject and determining the level in the sample of a biomarker for CFS/ME, wherein the biomarker is an expression product of a gene shown in Table 1. Genes in Table 1 have been found to be overexpressed in CFS/ME compared to unaffected individuals.

The method may comprise determining the level, in the sample, of a plurality of biomarkers (for example, 2, 3, 4, 5, 6, 7, 8, 9, 10, 15, 20 or more biomarkers), wherein each of the biomarkers is an expression product of a gene shown in Table 1.

The method may comprise the step of determining the level in the sample of one or more further biomarkers, being expression products of one or more genes from Table 6. Indeed, an expression product from any one of the genes of Table 6 may be used as a biomarker for CFS/ME, either alone or in combination with other genes of Table 6 or of Tables 1 to 5.

The method may be considered to provide an expression profile of the one or more biomarkers for CFS/ME, for the test subject, at the time of sampling. By “expression profile” is meant a set of data relating to the level of expression of one or more of the relevant biomarkers in a test subject, in a form which allows comparison with comparable expression profiles (e.g. from affected and/or unaffected individuals), in order to assist in the determination of whether or not the subject is affected by CFS/ME.

The method typically involves correlating the results obtained with a probability that the subject is affected by CFS/ME. In order to assist with this correlation, the method of the invention may comprise the step of comparing the expression profile for the test subject with one or more “reference” expression profiles, that is to say one or more expression profiles characteristic of unaffected subjects (i.e. subjects not suffering from CFS/ME), and/or one or more expression profiles characteristic of affected subjects (i.e. subjects not suffering from CFS/ME). Thus the level of expression of one or more of the said biomarkers in the test subject is typically compared with that characteristic of affected and/or unaffected individuals.

The reference expression profiles may be profiles previously derived from healthy or affected individuals, or may be artificial profiles which display expression levels of the relevant biomarkers which are characteristic of the relevant group. For example, they may be computer-generated from a plurality of profiles previously derived from appropriate individuals. The profile(s) characteristic of affected subjects may be divided into subgroups, e.g. according to their symptoms, as described in more detail below.

Taken alone, the expression profile for the test subject may not provide an absolute diagnosis of CFS/ME. Normally, a clinician will also take account of the physical and/or psychological symptoms of the subject in order to reach a diagnosis. However the expression profile generated by the methods of the invention provides useful data to help the clinician confirm or reject a preliminary diagnosis based on physical and psychological symptoms alone. A finding that one or more of these genes is upregulated in a particular individual may therefore provide support for a diagnosis of CFS/ME.

By “upregulated” or “overexpressed” is meant that the gene in question shows at least a two fold increase in expression at the level of mRNA and/or protein as compared to the level observed in unaffected (preferably healthy) individuals. If desired, the level of overexpression required to regard a result as positive may be set higher than this, e.g. a three, four, five, six, seven, eight, nine or ten fold increase as compared to unaffected individuals, or higher if required.

The genes in Table 1 not only provide biomarkers for CFS/ME, they also provide the first evidence of specific biochemical pathways which may be dysregulated in CFS/ME. They can be clustered into a number of subgroups, based on their involvement in the same or related biochemical pathways whose dysregulation is likely to play a role in either the underlying cause or the symptoms of the disease state. Previously it has not been possible to identify such pathways, which is a primary reason why no generally-recognised therapies exist for CFS/ME. The subgroups of genes identified by the present inventors provide an explanation for many of the symptoms displayed by those affected by CFS/ME, suggest rational therapies for the condition, and provide means for monitoring the efficacy of any therapy administered.

Without wishing to be bound by any particular theory, the inventors believe that CFS/ME is not a genetic disease caused by a single or multiple gene defects. Rather, they believe CFS/ME to be an acquired condition where there is a shift in the functional systems of a select number of genes regulating specific biological functions (e.g. infection and immunity, cell membrane function and cell cycle). Based on the expression patterns they have observed, they have established a model of “hub” and “network” genes which emphasises the interrelation of these key genes, with the hub genes being the control centre and the network genes being largely the effector arm of this functional system. This set of hub and network genes defines the functional shift in the biological systems of patients who continue to have symptoms due to CFS/ME.

Thus the genes of Table 1 are subdivided into “hub” genes and “network” genes. Essentially, in any particular pathway the hub gene(s) can be considered to lie upstream of the network genes, in that dysregulation of the hub gene is likely to lead (directly or indirectly) to dysregulation of the downstream genes. In Table 1, the hub genes are shown in section A, and the network genes in section B.

Certain genes which appear to be highly significant (e.g. based on the difference in their expression between the disease state and the normal state) have been allocated to the “hub” group even where no related set of downstream “network” genes is specifically identified.

Exemplary groups of genes identified in Table 1 are detailed below.

Defensin α1, CXCR4 and lactotransferrin (LTF) are all involved in the response to intracellular infection. The network genes associated with these hub genes include defensin α4, integrin α2B, integrin β3, arginase 1, arginase 2, thrombospondin 1, membrane associated protein 17 (MAP 17), Charcot Leyden Crystal Protein (CLC) and chondroitin sulphate proteoglycan 2 (versican). These genes are shown in Table 2.

Haemoglobin γ (foetal haemoglobin) is part of the oxidative stress response pathway, and is only expressed under conditions of oxidative stress. Network genes associated with the oxidative stress pathway include other haemoglobin genes including haemoglobin alpha 1, prostaglandin-endoperoxide synthase 1 and prostaglandin-endoperoxide synthase 2. These genes are shown in Table 3. However it will be noted that Table 6 includes genes for haemoglobins alpha, beta, gamma and delta. Any one of the haemoglobin genes of Table 6 may be used as a biomarker for CFS/ME, and may be used as well as, or in place of haemoglobin gamma or alpha if desired. For simplicity, though, reference will be made primarily to haemoglobin gamma.

Serine/threonine kinase 17B (STK17B) is implicated in apoptosis, as are the network genes caspase 1, dynamin 1-like, and phosphatidyl serine binding protein. These genes are shown in Table 4.

The MHC class II gene HLA-DRβ4 is upregulated in CFS/ME implying a shift from antigen presentation by MHC class I to MHC II presentation. Associated network genes include HLA-DQβ1 and the immunoglobulin heavy chain γ3 (IgG3). These genes are shown in Table 5. IgG1 may be used as a biomarker as well as, or in place of, IgG3.

Methods for investigating whether a test subject is affected by CFS/ME may therefore comprise determining the level of at least one gene from each of at least two of Tables 2, 3, 4 and 5, and preferably from each of three or four of said Tables. In preferred embodiments, at least one gene from each said Table is a hub gene.

Preferred genes of Table 1 are defensin α1, haemoglobin γ, CXCR4, tubulin beta 1 and HLA DRβ4. The method may involve testing expression of any one, two, three, four or all five of these genes, and optionally further hub genes of Table 1A.

Additionally or alternatively, the method may involve testing expression of one or more of TSP 1, caspase 1 and/or IgG3.

The method may also comprise the step of determining the level of an expression product of prostaglandin D2 synthase in sample. In contrast to the genes of Table 1, prostaglandin D2 synthase is found to be downregulated in individuals suffering from CFS/ME as compared to normal controls. Thus a finding that prostaglandin D2 synthase is downregulated in a particular individual may provide support for a diagnosis of CFS/ME.

By “downregulated” or “underexpressed” is meant that prostaglandin D2 synthase shows at least two fold higher expression at the level of mRNA and/or protein in unaffected (preferably healthy) individuals than is observed in the test subject. If desired, the level of downregulation required to regard a result as positive may require the level found in unaffected individuals to be three, four, five, six, seven, eight, nine or ten fold higher than in affected individuals.

The method may further involve the step of determining the level, in a biological sample from the subject, of the peptide QYNAD, as described in more detail below. The level of the peptide in the sample may form part of the expression profile established for the subject. An elevated level of the peptide as compared to unaffected individuals may be indicative of CFS/ME. By an “elevated” level is meant at least twice the level found in unaffected healthy controls, and preferably at least 5 times or at least 10 time the level found in unaffected healthy controls.

It will be appreciated that individual patients who each satisfy the criteria for CFS/ME may nevertheless present a range of very different symptoms. Some of these can be explained by the groups of genes identified herein.

For example, CFS/ME has previously been suggested to involve immune dysfunction. Some CFS/ME sufferers are particularly prone to infection by viruses (e.g. influenza) and other pathogens; indeed some suffer recurrent infections. Others are affected by atopic/allergic symptoms. These symptoms may be associated with the apparent shift from Type I to Type II antigen presentation by the MHC, which could impair the body's ability to deal efficiently with infections and could also exacerbate allergy/atopy.

Some CFS/ME sufferers describe their body as feeling prematurely aged, and can display restricted mobility characteristic of much older individuals. This might be explained by excessive apoptosis, particularly in the central nervous system. Therefore patients having these symptoms might be expected to show increased expression of serine/threonine kinase 17B (STK17B) and its associated network genes.

Oxidative stress is also a cause of apoptosis, particularly in the nervous system. Thus patients with increased expression of haemoglobin γ and its associated network genes may also show, or be at risk of, accelerated programmed cell death (increased level of apoptosis), particularly neuronal apoptosis. Features of oxidative stress and neuronal apoptosis may include ageing, cognitive impairment and chronic pain.

The genes of Table 2 are implicated in cellular protection (“defence”) against viral or bacterial infections. Thus an overexpressed defensin gene in CFS/ME would be consistent with immune activation and correlate with symptoms of recurrent influenza-type symptoms, sore throat and lymph node enlargement experienced by patients with CFS/ME.

In a further aspect, the invention therefore provides a method for classifying a subject affected by CFS/ME, the method comprising providing a biological sample from the subject and determining the level in the sample of a biomarker for CFS/ME, wherein the biomarker is an expression product of a gene shown in one or more of Tables 2 to 5.

Preferably the biomarker is a hub gene from one of Tables 2, 3, 4 or 5. More preferably, the method comprises determining the expression level of hub genes from each of two, three or all four of Tables 2, 3, 4 or 5. The method may also comprise determining network genes from one, two, three or all four of Tables 2, 3, 4 and 5.

The individual can then be assigned to a subgroup of CFS/ME, dependent on which group or groups of genes are found to be upregulated. Thus, for example, the subject may be classified as being affected by one or more of oxidative stress, excessive apoptosis, and immunological dysregulation (MHC I to II shift). The assignment step may involve comparing the expression profile obtained from the subject with one or more expression profiles characteristic of individuals previously assigned to one or more subgroups of CFS/ME.

It will be appreciated that the classification may be performed using the same expression profile as that established for determining whether the subject is affected by CFS/ME.

Individuals in which these particular pathways are dysregulated may be suitable for treatment by the methods described below. Therefore in a further aspect the invention provides a method of determining whether an individual affected by CFS/ME is suitable for treatment using such a therapy. The method comprises determining whether the individual is affected by one or more of oxidative stress, excessive apoptosis, and immunological dysregulation as described above, and optionally prescribing a suitable treatment depending on the outcome. These treatments are described in detail below.

Expression of individual biomarkers for CFS/ME (genes from Tables 1 to 5) may differ slightly between independent samples, leading to slightly different expression profiles for individual samples. However, these particular genes may provide a characteristic pattern of expression (expression profile) in an affected individual (i.e. one suffering from CFS/ME) that is recognisably different from that in an unaffected individual (i.e. one not suffering from CFS/ME).

By creating a number of expression profiles from a number of known affected and unaffected samples, it is possible to create a library of profiles for both sample types. The greater the number of expression profiles, the easier it is to create a reliable characteristic expression profile standard (i.e. including statistical variation) that can be used as a control in a diagnostic assay. Thus, a standard profile may be one that is devised from a plurality of individual expression profiles and devised within statistical variation to represent either the affected or unaffected profile.

The determination of the expression profile may be computerised and may be carried out within certain previously set parameters, to avoid false positives and false negatives.

The computer may then be able to provide an expression profile standard characteristic of an affected sample and a normal sample. The determined expression profiles may then be used to classify test samples as affected or unaffected as a way of diagnosis.

Thus, in a further aspect the invention provides a method of creating a library of expression profiles for use in determining whether an individual is affected by CFS/ME, the method comprising

(a) providing biological samples from a plurality of individuals affected by CFS/ME, and determining the level in each sample of one or more biomarkers for CFS/ME to create a plurality of expression profiles from affected individuals;
(b) providing biological samples from a plurality of individuals not affected by CFS/ME, and determining the level in the sample of said one or more biomarkers for CFS/ME to create a plurality of expression profiles from unaffected individuals;
wherein the biomarkers comprise expression products of one or more genes shown in Table 1.

Typically, the method comprises the step of retrievably storing each of the expression profiles on a computer data carrier, in order to create a database of expression profiles for both affected and unaffected individuals.

The invention further provides an expression profile database, comprising a plurality of expression profiles of biomarkers for CFS/ME from affected and unaffected individuals, wherein the biomarkers comprise expression products of one or more genes shown in Table 1.

It will be appreciated that the profiles may be classified according to their expression levels of the various groups of genes shown in Tables 2 to 5 as already described. The combinations of biomarkers analysed in establishing the expression profiles may therefore be chosen as described above.

The expression profiles may comprise data relating to the level, in biological samples from the subjects, of the peptide QYNAD. If required, the methods may therefore comprise the step of determining the level of this peptide in biological samples from the subjects.

At present there are no generally acknowledged treatments for CFS/ME. One problem which has faced researchers attempting to develop and evaluate suitable therapies is the lack of objective testable criteria to determine their efficacy. The present invention provides a useful tool for monitoring the efficacy of an interventional treatment for CFS/ME, by studying the effect of the therapy, over time, on an individual's expression profile of the biomarkers described in Tables 1 to 5. The methods described may therefore be used to evaluate the effectiveness of a test treatment, e.g. by testing it on a population of subjects affected by CFS/ME, or to investigate an individual's response to a particular therapy, to see whether or not they are responding appropriately.

Thus in a further aspect the invention provides a method of determining the efficacy of a treatment for CFS/ME comprising the steps of:

(a) providing a biological sample from a subject affected by CFS/ME who has been subjected to said treatment,
(b) determining the level in said sample of one or more biomarkers for CFS/ME to create an expression profile for said subject, and
(c) comparing said expression profile with
i) a comparable expression profile obtained from said test subject before initiation of said treatment, and/or
ii) a comparable expression profile obtained from said test subject at an earlier stage of said treatment, and/or
iii) a comparable expression profile characteristic of a subject who is unaffected by CFS/ME,
wherein the one or more biomarkers for CFS/ME are expression products of one or more genes shown in Tables 1 to 5. Preferred combinations of biomarkers have already been described in relation to earlier aspects of the invention.

In general, a treatment may be considered to be effective if the subject's expression profile after treatment shows that the expression level of one or more of the biomarkers is reduced compared to its level before treatment, or its level earlier in a course of the treatment. Preferably the subject's expression profile after treatment approaches a profile characteristic of an individual unaffected by CFS/ME. That is to say, the expression level of each of the biomarkers falls within the normal range found in unaffected individuals.

Some treatments may be targeted to particular pathways known to be dysregulated in CFS/ME. In such cases, it may be desirable to follow the effect of the treatment on the expression levels of genes in the pathway likely to be modulated by that treatment. For example, a treatment which is intended to reduce oxidative stress, apoptosis or immune dysfunction (characterised by MHC shift) may have a particular effect on the expression levels of the genes in Tables 3, 4 and 5 respectively. The biomarkers chosen to study the effects of the treatment may be chosen accordingly. Preferred combinations of biomarkers from these Tables have already been described.

The method may further comprise determining the effect of the treatment on the level of the peptide QYNAD in a biological sample from the test subject.

In the diagnostic and analytical methods described herein, the expression levels of the chosen biomarker(s) for CFS/ME are preferably determined using peripheral blood mononuclear cells (PBMCs) from the test subject. Therefore the biological sample used to establish the expression profile is preferably a blood sample or cells isolated from a blood sample. If desired, the method may comprise the steps of enriching PBMCs in the sample, or isolating PBMCs from the sample.

It will be understood that the methods described are generally performed on a biological sample which has been isolated from the test subject. The method may, but need not, comprise the actual step of isolating the sample from the test subject, e.g. by taking a blood sample.

Typically, the expression level of each of the biomarkers is determined by contacting the sample with a binding agent capable of binding specifically to an expression product of the genes encoding that biomarker (a gene of Table 1, 2, 3, 4, 5 or 6). Binding between the agent and an expression product present in the sample is then determined. If it is desired to determine the expression level of more than one gene, then the sample may be contacted with a plurality of binding agents, either simultaneously or sequentially, each binding agent being capable of binding specifically and individually to an expression product of one of the biomarker genes.

Binding agents capable of binding to nucleic acid expression products (e.g. mRNA, pre-mRNA) are typically nucleic acid primers or probes having a sequence of, or complementary to, a portion of the nucleic acid expression product. This enables the binding agent to hybridise under suitable conditions with the nucleic acid expression product itself, e.g. in a Northern blot or in situ hybridisation assay, or to a cDNA copy of the nucleic acid expression product, e.g. in a RT-PCR assay, Southern blot or microarray assay.

Binding agents capable of binding to polypeptide expression products include ligands and receptors for the polypeptide in question. Particularly preferred examples of binding agents are antibodies specific for (e.g. raised against) the desired polypeptide, or fragments thereof comprising an antigen binding site. These may be used in a variety of immunological assay methods, including Western blots and ELISA assays, as well as in microarray assays.

It may be desirable to isolate expression products or particular fractions thereof (e.g. total RNA, mRNA, total protein, soluble proteins or membrane proteins) from the sample before contacting them with the binding agent. The extent to which this is necessary will vary depending on the chosen assay method.

Simple immunological assays (such as ELISA assays) or PCR-based assays using ten biomarkers or less (e.g. 2, 3, 4, 5, 6, 7, 8, 9 or 10 biomarkers) are particularly preferred, because they can readily be adapted to analyse large numbers of samples in a relatively short space of time, at relatively low cost. Such assays are also well-suited to automation. Preferred markers and combinations of markers are as described elsewhere in this specification.

The diagnostic and analytical methods described above may also comprise determining the level, in a biological sample from the subject, of the peptide QYNAD. It is believed that the peptide may be found in serum and cerebrospinal fluid (CSF) in most or all of the population, but it is found at elevated levels in subjects suffering from inflammatory and immunological disorders of the nervous system (such as multiple sclerosis and Guillain-Barre syndrome). The present inventors have now established that it is also found at significantly elevated levels in subjects suffering from CFS/ME.

The biological sample used for determining the peptide level may be a blood sample (or serum derived therefrom). Thus, conveniently, the same blood sample may be used for determining levels of the peptide and the other biomarkers of CFS/ME. Alternatively, a different sample may be used, in particular, a sample of cerebrospinal fluid (CSF) where available.

The level of the peptide may be determined as described by Brinkmeier et al.¹⁶, e.g. by gel filtration chromatography. Alternatively conventional immunoassays such as ELISAs or Western blots may be used, employing antibodies raised against the peptide.

The assay for the peptide is generally performed on a sample isolated from the test subject. The method of the invention need not comprise the step of actually isolating the sample from the test subject.

In a further aspect, the invention provides a kit for use in a diagnostic or analytical method as described herein, the kit comprising a plurality of binding agents, each capable of binding specifically and individually to an expression product of one of the genes of Table 1, or the peptide QYNAD. Thus the kit includes binding agents specific for expression products of two or more genes of Tables 1, or at least one of the genes of Table 1 and the peptide QYNAD. The binding agents provided in the kit may be capable of binding specifically and individually to expression products of 2, 3, 4, 5, 6, 7, 8, 9, 10, 15, 20 or more of the genes of Table 1 and optionally the peptide QYNAD.

The kit is suitable for use in the methods of the invention described in this specification, and may comprise instructions for performing one or more methods of the invention.

The binding agents may be immobilised on one or more solid supports. Discrete supports may each carry only one type of binding agent. For example, distinct populations of beads may each carry one type of binding agent. Alternatively, a single support may carry more than one type of binding agent. Indeed, one support (e.g. a microarray chip) may carry all of the different types of binding agent provided with the kit.

In addition, the kit may comprise one or more binding agents capable of binding specifically to an expression product of a control gene which is not differentially expressed between individuals affected and unaffected by CFS/ME. The level of expression from this control gene may be measured in order to assist in quantification of the expression products of the genes of Table 1, and/or for quality assurance of an assay performed using the kit. Preferably a control gene is chosen which is constitutively expressed in the cells of the biological sample (i.e. always expressed, at substantially the same level, under substantially all conditions). Such genes are often referred to as “housekeeping” genes. Examples include glyceraldehyde phosphate dehydrogenase (GAPDH), β-actin, and abl (ableson tyrosine kinase).

The kit may comprise yet further binding agents capable of binding to expression products of other biomarker genes or control genes. However, in preferred embodiments, the kit comprises binding agents for expression products of less than 1000 different genes, e.g. less than 500 different genes, less than 100, less than 50, less than 40, less than 30, less than 20, or less than 10 different genes.

As explained above, the groups of genes identified by the present inventors also suggest specific therapies for CFS/ME.

Patients displaying signs of oxidative stress may be treated with an anti-oxidant. Thus the invention provides a method of treating CFS/ME in an individual suffering therefrom, comprising administering an effective amount of an anti-oxidant.

Therapeutically effective amounts of a corticosteroid and/or minocyline may also be administered to the subject in conjunction with the anti-oxidant. As will be appreciated, the individual active agents may be administered individually (in two or more separate compositions) or together (in the same composition).

The invention further provides the use of an anti-oxidant in the preparation of a medicament for the treatment of CFS/ME. The medicament may be formulated for administration in conjunction with a corticosteroid and/or minocycline. Alternatively, the medicament may comprise a corticosteroid and/or minocycline.

Examples of suitable anti-oxidants include coenzyme Q10 and inhibitors of cyclooxygenase (COX) enzymes, particularly COX II enzymes, such as celecoxib (4-[5-(4-methylphenyl)-3-(trifluoromethyl)-1H-pyrazol-1-yl] benzenesulfonamide).

Patients showing evidence of abnormal apoptosis may be treated with minocyline, which is an inhibitor of caspase 1, shown here to be upregulated in individuals affected by CFS/ME. Minocyline has previously been suggested for treatment of various neurological disorders including stroke, multiple sclerosis, spinal cord injury, Parkinson's disease, Huntington's disease and amylotrophic lateral sclerosis—see Wee Young et al., Lancet Neurology, 2004, 744-751 for review.

Thus the invention provides a method of treating CFS/ME in an individual suffering therefrom, comprising administering an effective amount of minocycline.

Therapeutically effective amounts of a corticosteroid and/or an anti-oxidant may also be administered to the subject in conjunction with the minocycline. As will be appreciated, the individual active agents may be administered individually (in two or more separate compositions) or together (in the same composition).

The invention further provides the use of minocycline in the preparation of a medicament for the treatment of CFS/ME. The medicament may be formulated for administration in conjunction with a corticosteroid and/or an anti-oxidant. Alternatively, the medicament may comprise a corticosteroid and/or an anti-oxidant.

Oxidative stress often gives rise to apoptosis. Therefore it may be advisable to treat patients showing signs of oxidative stress with an apoptosis treatment such as minocycline as a precautionary measure if they do not already show upregulation of genes involved in apoptosis.

Patients showing signs of immune dysfunction, and particularly the Type I to II shift of MHC expression, may benefit from treatment with a corticosteroid. This would address the hypocortisolism previously reported in some CFS/ME patients.

Thus the invention provides a method of treating CFS/ME in an individual suffering therefrom, comprising administering an effective amount of a corticosteroid.

Therapeutically effective amounts of minocycline and/or an anti-oxidant may also be administered to the subject in conjunction with the corticosteroid. As will be appreciated, the individual active agents may be administered individually (in two or more separate compositions) or together (in the same composition).

The invention further provides the use of a corticosteroid in the preparation of a medicament for the treatment of CFS/ME. The medicament may be formulated for administration in conjunction with minocycline and/or an anti-oxidant. Alternatively, the medicament may comprise minocycline and/or an anti-oxidant.

An example of a suitable corticosteroid is hydrocortisone. Others include dexamethasone and prednisone.

The invention further provides pharmaceutical compositions suitable for the treatment of CFS/ME. Thus there is provided a pharmaceutical composition comprising a therapeutically effective amount of minocycline in combination with a therapeutically effective amount of a corticosteroid and/or an anti-oxidant and a pharmaceutically acceptable carrier.

Also provided is a pharmaceutical composition comprising a therapeutically effective amount of a corticosteroid in combination with a therapeutically effective amount of minocycline and/or an anti-oxidant, and a pharmaceutically acceptable carrier.

Also provided is a pharmaceutical composition comprising a therapeutically effective amount of an anti-oxidant in combination with a therapeutically effective amount of a corticosteroid and/or minocycline, and a pharmaceutically acceptable carrier.

As described above, a particular treatment may be determined for any individual based on their expression profile of CFS/ME biomarkers. For example, an individual showing upregulation of oxidative stress pathway genes may be particularly suitable for treatment with an anti-oxidant. However it may not be convenient or necessary to establish a suitable expression profile before beginning treatment.

Therefore it may be desirable to administer one, two or all three of the treatment types described above to an individual affected by CFS/ME without recourse to expression profiling.

### DESCRIPTION OF THE DRAWINGS

FIG. 1 shows a RT-PCR assay of v-ATPase mRNA in PBMCs from three healthy control individuals (lanes 2 to 4) and three patients with CFS/ME (lanes 5 to 7). Lane 1 contains molecular weight markers.

FIG. 2 shows a RT-PCR assay of Defensin alpha 1 mRNA in PBMCs from three healthy control individuals (lanes 2 to 4) and three patients with CFS/ME (lanes 6 to 8). Lanes 1 and 9 contain molecular weight markers.

FIGS. 3 and 4 show Western blots of proteins extracted from PBMCs from healthy individuals and individuals suffering from CFS/ME, stained with antibodies against Defensin alpha 1 and Thrombospondin 1 respectively. In each case, lanes 1 and 12 contain molecular weight markers, lanes 2 to 11 contain samples from patients with CFS/ME, and lanes 13 to 22 contain samples from healthy control individuals. Purified protein extract from 2×10⁵ cells was loaded per lane. Proteins were separated on 10% SDS-PAGE gels and blotted onto a nylon membrane.

FIG. 5 shows a SDS-PAGE gel, stained with Coomassie blue, of the same samples used to prepare the Western blots shown in FIGS. 3 and 4. Lanes 11 and 22 contain molecular weight markers, lanes 1 to 10 contain samples from patients with CFS/ME, and lanes 12 to 21 contain samples from healthy control individuals. As in FIGS. 3 and 4, protein extract from 2×10⁵ cells was loaded per lane.

### DETAILED DESCRIPTION OF THE INVENTION

### Chronic Fatigue Syndrome/Myalgic Encephalomyelitis

CFS is typically diagnosed using the modified CDC criteria described by Fukuda et al.². All other conditions or diseases which could explain a patient's symptoms are first excluded. Having done this, CFS/ME is diagnosed if the patient has been affected by 6 months or longer of persistent relapsing or persistent fatigue accompanied by four or more concurrent symptoms including impaired memory severe enough to affect normal daily function, sore throat, tender lymph nodes, muscular or joint pain, new headaches, unrefreshing sleep and post-exertional malaise lasting for more than 24 hours. For the purposes of this specification, individuals satisfying these criteria are considered to be affected by CFS/ME.

### Assay Methods

The genes identified in Table 1 provide biomarkers which may be used in diagnostic assays to support, confirm, or refute a diagnosis of CFS/ME. With the knowledge of this set of genes, it is possible to devise many methods for determining a suitable expression profile of one or more CFS/ME biomarkers in a particular test sample.

Typically, the method involves contacting expression products from the sample with a binding agent capable of binding to an expression product of a gene identified in Table 1. The expression product may be a transcribed nucleic acid sequence or an expressed polypeptide.

The transcribed nucleic acid sequence may be mRNA or pre-mRNA. Alternatively, the expression product may also be cDNA produced from said mRNA. The binding member may a nucleic acid having a sequence complementary to that of the RNA or cDNA which is consequently capable of specifically binding to the transcribed nucleic acid or cDNA under suitable hybridisation conditions, e.g. by Northern blotting, in situ hybridisation, or Southern blotting.

Such protocols may use probes of at least about 20-80 bases in length. The probes may be of 100, 200, 300, 400 or 500 bases in length or more. Binding assays may be conducted using standard procedures, such as described in Sambrook et al., Molecular Cloning A Laboratory Manual (New York: Cold Spring Harbor Laboratory Press, 1989 or later editions).

RT-PCR procedures (including quantitative PCR procedures) may also be used to analyse the presence or amount of mRNA or precursor mRNA in a given sample. A suitable primer having at least 15 to 20 bases complementary to the desired mRNA or precursor mRNA sequence will typically be used to prime cDNA synthesis. Alternatively a poly-T primer (optionally comprising one or more random nucleotides at the 3′ end) may be used to prime cDNA synthesis from all mRNA in the sample.

Subsequently, a segment of the cDNA is amplified in a PCR reaction using a pair of nucleic acid primers, each typically having at least 15 to 20 bases complementary to the desired RNA sequence. The skilled person will be able to design suitable probes or primers based on the publicly available sequence data for the genes in question (see Table 1 for suitable accession numbers).

Where the expression product is the expressed polypeptide, the binding member is preferably an antibody raised against or otherwise specific for the desired polypeptide, or any other molecule comprising the antigen binding site from such an antibody.

The skilled person will realise that other binding agents may be used as appropriate. Suitable agents may include naturally-occurring ligands and receptors for the desired polypeptide, aptamers, etc. For example, aptamers are nucleic acid molecules (typically DNA or RNA), selected from libraries on the basis of their ability to bind other molecules. Aptamers have been identified which can bind to other nucleic acids (by means other than conventional Watson-Crick base pairing), proteins, small organic compounds, and even entire organisms.

The binding agent (e.g. a nucleic acid probe or antibody) may be fixed to a solid support. The expression products may then be passed over the solid support, thereby bringing them into contact with the binding agent. Conveniently, the binding agents are immobilised at defined, spatially separated locations, to make them easy to manipulate during the assay. The solid support may be a glass surface, e.g. a microscope slide, beads, fibre-optics or microarray chip. In the case of beads, each binding agent may be fixed to an individual bead and they may then be contacted with the expression products in solution.

The sample is generally contacted with the binding agent(s) under appropriate conditions which allow the analyte in the sample to bind to the binding agent(s). The fractional occupancy of the binding sites of the binding agent(s) can then be determined.

Whatever the chosen assay system, there are numerous ways to detect interaction between the binding agent and the expression product (analyte) to be determined, either by directly or indirectly labelling the analyte or binding agent, or by using a developing agent to arrive at an indication of the presence or amount of the analyte in the sample. A developing agent may be a secondary binding agent, capable of binding to a complex between analyte and primary binding agent. For example, if a primary antibody is used as a binding agent, the developing agent may be a secondary antibody capable of binding either to the primary antibody, or to a different epitope on the analyte to that recognised by the primary antibody.

Typically, the analyte, binding agent or developing agent is directly or indirectly labelled (e.g. with radioactive, fluorescent or enzyme labels, such as horseradish peroxidase) so that they can be detected using techniques well known in the art. Directly labelled agents have a label associated with or coupled to the agent. Indirectly labelled agents may act on a further species to produce a detectable result.

Thus, radioactive labels can be detected using a scintillation counter or other radiation counting device, fluorescent labels using a laser, confocal microscope, etc., and enzyme labels by the action of an enzyme label on a substrate, typically to produce a colour change. In further embodiments, the developing agent or analyte is tagged to allow its detection, e.g. linked to a nucleotide sequence which can be amplified in a PCR reaction to detect the analyte. Other labels are known to those skilled in the art are discussed below.

The developing agent(s) can be used in a competitive method in which the developing agent competes with the analyte for occupied binding sites of the binding agent, or non-competitive method, in which the labelled developing agent binds analyte bound by the binding agent or to occupied binding sites. Both methods provide an indication of the number of the binding sites occupied by the analyte, and hence the concentration of the analyte in the sample, e.g. by comparison with standards obtained using samples containing known concentrations of the analyte.

In alternative embodiments, the analyte can be tagged before applying it to the support comprising the binding agent.

There is an increasing tendency in the diagnostic field towards miniaturisation of such assays, e.g. making use of binding agents (such as antibodies or nucleic acid sequences) immobilised in small, discrete locations (microspots) and/or as arrays on solid supports or on diagnostic chips. These approaches can be particularly valuable as they can provide great sensitivity (particularly through the use of fluorescent labelled reagents), require only very small amounts of biological sample from individuals being tested and allow a variety of separate assays can be carried out simultaneously. This latter advantage can be useful as it provides an assay employing a plurality of analytes to be carried out using a single sample. Examples of techniques enabling this miniaturised technology are provided in WO84/01031, WO88/1058, WO89/01157, WO93/8472, WO95/18376/WO95/18377, WO95/24649 and EP 0 373 203 A.

Other methods which do not rely on labelling techniques may also be used to detect interaction between binding agent and reporter molecule, including physical methods such as surface plasmon resonance, agglutination, light scattering or other means.

Expressed nucleic acid (mRNA, pre-mRNA) can be isolated from the cells using standard molecular biological techniques. The expressed nucleic acid sequences corresponding to the gene or genes of Table 1 can then be amplified using nucleic acid primers specific for the expressed sequences in a PCR, e.g. real time PCR, multiplex PCR, etc. The skilled person will be able to select or design a suitable reaction type and protocol depending on, e.g. the number and particular combination of genes to be analysed. If the isolated expressed nucleic acid is mRNA, this can be converted into cDNA for the PCR reaction using standard methods.

The primers may conveniently introduce a label into the amplified nucleic acid so that it may be identified. Ideally, the label is able to indicate the relative quantity or proportion of nucleic acid sequences present after the amplification event, reflecting the relative quantity or proportion present in the original test sample. For example, if the label is fluorescent or radioactive, the intensity of the signal will indicate the relative quantity/proportion or even the absolute quantity, of the expressed sequences. The relative quantities or proportions of the expression products of each of the genes of Table 1 may be used to establish a particular expression profile for the test sample.

Other methods for detection of nucleic acid expression products may also be used, such as in situ hybridisation, Northern blot, etc.

Likewise, protein expression products may be detected by any suitable technique. Immunological techniques are particularly preferred, in which antibodies specific for the particular polypeptide gene product(s), are used as binding agents, although other binding agents such as receptors or ligands capable of binding to the proteins of interest may be employed.

In some embodiments, protein expression products from the sample under test are immobilised on a solid phase and contacted with a binding agent specific for one or more of the proteins of Table 1 under appropriate conditions which allow binding between the protein and the binding agent. The amount of the binding agent found at the surface is then determined. For example, the binding agent may be directly labelled. Alternatively, the immobilised antibody may be contacted with a labelled developing agent capable of binding to the primary antibody. Examples of this type of assay include Western blotting, and certain ELISA (enzyme-linked immunosorbent assay) techniques.

In other embodiments, a binding agent is immobilised on a solid phase and contacted with the sample under suitable conditions to allow binding to take place. The fractional occupancy of the binding sites of the binding agent(s) can then be determined either by directly or indirectly labelling the analyte or by using a developing agent or agents to arrive at an indication of the presence or amount of the analyte in the sample.

An example of this type of assay is an antibody sandwich assay (e.g. an ELISA), which employs two antibodies each capable of binding to a different site on the biomarker protein. The first is immobilised on a solid phase for use as the binding agent. After contact with the analyte, the second antibody is used to detect complexes between the first antibody and analyte.

Whichever method is chosen, it is important that the assay provides a read-out of the level of expression of the biomarker genes which allows results from different individuals to be compared reliably with one another. By way of example, the level of a particular expression product may be determined as a proportion of the total expression products found in the sample. Alternatively, the level of a particular expression product may be determined in relation to the level of expression of a control gene such as a housekeeping gene, or the like. Alternatively, it may be convenient to determine the absolute amount of a particular expression product, e.g. by comparison with known standards. The skilled person will be capable of designing a suitable protocol for any given assay method, and will also be aware of other suitable embodiments.

### Antibodies

It has been shown that fragments of a whole antibody can perform the function of binding antigens. The term “antibody” is therefore used herein to encompass any molecule comprising the binding fragment of an antibody, and the term binding agent and binding site should be construed accordingly. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988). In preferred embodiments the binding agent comprises a single antigen binding site specific for the analyte, i.e. a monovalent antibody or antibody fragment.

### Pharmaceutical Compositions

Pharmaceutical compositions as described in this specification typically comprise, in addition to one or more suitable active agents, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Whether it is a polypeptide, antibody, peptide, nucleic acid molecule, small molecule or other pharmaceutically useful compound that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

A composition may be administered alone or in combination with other treatments, either simultaneously or sequentially dependent upon the condition to be treated.

### EXAMPLES

A group at the University of Ulm, Germany, has recently suggested that a pentapeptide (QYNAD) with Na⁺ channel-blocking function could be a biological marker of certain inflammatory and immunological disorders of the nervous system¹⁶.

The inventors asked whether or not the pentapeptide identified by the German group¹⁶ might play a role in CFS. Samples of serum were sent to the University of Ulm for analysis. The 15 samples included 5 normal controls, 5 patients with CFS and 5 disease controls including two patients with MS. Samples were numbered 1-15 and the German group were not informed what the samples were, or which samples were which, until the experiment was concluded.

When the code was broken, the results showed that the disease control group had levels of the pentapeptide which were 2.3× those of the normal controls (similar to the published data) and the CFS samples had levels which were 3× higher than the healthy controls.

Thus, there are measurably higher amounts of the pentapeptide in patients with CFS compared with healthy controls. Although the pentapeptide may not be specific to CFS (as high levels are also found in other disorders), an assay for the peptide could be used as part of the differential diagnosis of CFS.

The German group was unable to identify an endogenous gene which encodes the pentapeptide. The inventors carried out NCBI BLAST and EMBL-Heidelberg Bioccelerator amino acid alignments for the pentapeptide QYNAD. A total of one hundred alignment hits were found. Of these, only nine showed 100% similarity over the five amino acids—five of those were human. The amino acid searches were followed by NCBI BLAST searches using the GenBank Accession and gi numbers for each of the five human amino acid to determine their origins, references and nucleotide sequences. A number of cloned nucleotide sequences were found and when these were run through the nucleotide databases, only one clone showed full-length homology to any human gene. This gene was a human ion-channel gene—the vacuolar proton pump H⁺-ATPase (v-ATPase). Remarkably, when the human gene amino acid sequence was compared with the original QYNAD pentapeptide it was discovered that the relevant part of the human ion channel encodes the sequence QYMAD.

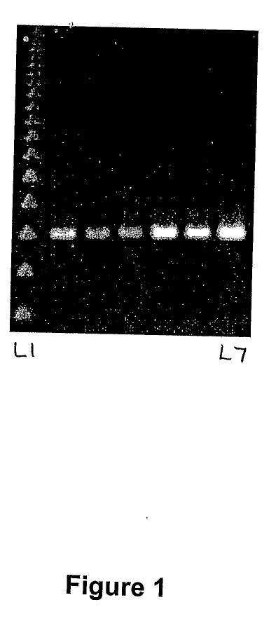

The inventors next asked whether the v-ATPase represents a candidate gene for a diagnostic test for CFS. RT-PCR using primers specific for the v-ATPase was performed on cDNA prepared from mRNA from PBMCs from CFS patients and healthy controls. As shown in FIG. 1, the patient samples have a significantly higher level of v-ATPase mRNA than the healthy controls. Thus the v-ATPase gene appears to represent a genuine biomarker for CFS/ME.

The vATPase is known to be involved in regulation of a number of metabolic functions which are deranged in CFS/ME. vATPase upregulation could therefore provide an explanation for a number of the symptoms observed. For example, increased vATPase activity could explain the intracellular acidosis in exercising muscles, chest pain (syndrome X), altered neurotransmitter (dopamine) function and abnormal regulation of hypothalamic hormones. In addition, it could explain the increased energy expenditure and fatigue associated with the condition. Taken together, this suggests that the vATPase is not only a marker for the condition, but is a realistic target for intervention therapy.

### Other Biomarkers for CFS/ME

The inventors went on to examine whether the increase in vATPase expression was confirmed by microarray analysis. Such analysis provides the opportunity to examine the differential expression of mRNA from a very large number of genes. Surprisingly, the results of the analysis not only confirmed their earlier findings regarding the vATPase gene, but also identified differences in the level of expression of key genes in the PBMC of patients with CFS/ME and control subjects, giving an insight into the biochemical pathways which are involved in this disorder.

Microarray results have been verified by western blot analysis and RT-PCR assay. A number of genes, in addition to v-ATPase, were significantly up/downregulated and identified as suitable biomarkers for the disorder.

Advances in genome sequencing and automated chip manufacture have made DNA chip or microarray technology readily available²⁵. This technology allows simultaneous differential expression profiling from a very large number of genes in tissue samples of CFS/ME patients and controls. A recent report from Vernon et al (2002), described a CFS biomarker search in PBMC using a DNA chip array assay which included 1,764 genes. In the study reported here, RNA isolated from PBMC, was assayed using Affymetrix genome-wide chips (HG-U133 arrays) which included 30,000 gene sequences.

Using DNA microarray analysis of whole human genome, gene transcriptional signatures were compared in the PBMC of eight male patients with CFS and seven age-matched male healthy controls. An additional cohort of fourteen patients with CFS and age and sex matched controls was recruited for RT-PCR and western blot assays in order to verify the microarray data. Analysis of the microarray data was performed as described previously (Breitling R. Armengaud P. Amtmann A. Herzyk P. Rank products: A simple, yet powerful, new method to detect differentially regulated genes in replicated microarray experiments. FEBS Letters 2004; 573(1-3): 83-92; Breitling R. Amtmann A. Herzyk P. Iterative Group Analysis (iGA): A simple tool to enhance sensitivity and facilitate interpretation of microarray experiments. BMC Bioinformatics 2004; 5: (pp 8p)).

Genes which are significantly upregulated in CFS/ME patients compared to healthy controls are detailed in Table 6, ranked according to their RP values. It is considered that any of these genes may be used as biomarkers for CFS/ME. Particularly preferred marker genes are detailed in Table 1.

Further genes, including prostaglandin D2 synthase and T-cell receptors alpha, beta, gamma and delta are found to be down-regulated in CFS patients compared to normal controls. Prostaglandin D2 synthase (NCBI accession no. BC 005939, UniGene Hs.446429) is considered to be a good candidate for a CFS/ME biomarker, because it is known to be involved in sleep regulation; patients with CFS frequently suffer from sleep reversal and fatigue associated with lack of sleep. However, data for other downregulated genes is not shown here. In general, genes which are upregulated in the disease state are considered to be better biomarkers for diagnostic tests etc. than genes which are downregulated because the potential for false-positive tests is significantly higher when using genes which are underexpressed in the disease state.

Iterative group analysis of the differentially expressed genes indicate that in CFS, there is a shift of immune response with preferential antigen presentation to MHC class II receptors and downregulation of T-cell receptor-α, increased cell membrane prostaglandin-endoperoxide synthase activity with downstream changes in oxygen transport and also activation of the guanyl cyclase and caspase pathways of cellular apoptosis. Another set of genes was identified which are involved in the immediate response to infection, particularly by intracellular parasites. The particular genes involved in each of these pathways are identified in Tables 2 to 5. In each of these key pathways, the hub genes were higher ranked in the analysis compared to the network genes.

Functional changes produced by altered gene regulation may explain the mechanism of fatigue and offer a rational basis for targeted pharmacotherapy in CFS.

TABLE 1
Biomarker Genes for CFS/ME

| | | |
|---|---|---|
| NCBI Accession Number | Gene | Gene symbol |

Table 1A - Hub Genes

| | | |
|---|---|---|
| NM_002343 | Defensin α 1 | DEFA 1 |
| AI_133353 | Haemoglobin γ | HBG1 |
| L_01639 | Chemokine (c-x-c) | CXCR4 |
| | receptor 4 | |
| NM_002343 | Lactotransferrin | LTF |
| NM_004226 | Serine/threonine | STK17B |
| | kinase 17B | |
| BC005312 | Major | HLA-DRB4 |
| | Histocompatibility | |
| | Complex Class II DR | |
| | β4 | |
| NM_030773 | Tubulin beta 1 | TUBB1 |
| M_87789 | Immunoglobulin heavy | IGHG3 |
| | constant γ3 | |
| NM_000570 | CD16 (IgG Fc | FCGR3A |
| | receptor, low | |
| | affinity IIIa) | |

Table 1B - Network Genes

| | | |
|---|---|---|
| NM_000558 | Haemoglobin α 1 | HBA1 |
| NM_001925 | Defensin α 4 | DEFA 4 |
| U13699 | Caspase 1 | CASP 1 |
| NM_000419 | Integrin α 2B, CD41B | ITGA2B |
| M35999 | Integrin β 3 CD61 | ITGB3 |
| NM_005764 | Membrane associated | MAP 17 |
| | protein 17 | |
| NM_001828 | Charcot Leyden | CLC |
| | Crystal Protein | |
| S36219 | Prostaglandin- | PTGS1 |
| | endoperoxide | |
| | Synthase 1 | |
| NM_000963 | Prostaglandin- | PTGS2 |
| | endoperoxide | |
| | Synthase 2 | |
| M16276 | Major | HLA-DQB1 |
| | Histocompatibility | |
| | Complex Class II | |
| | DQβ1 | |
| NM_012062 | Dynamin 1 | DNM1L (357) |
| NM_004657 | Phosphatidyl serine | SDPR (69) |
| | binding prot. Serum | |
| | deprivation response | |
| NM_000045 | Arginase 1 | ARG1 (358) |
| U75667 | Arginase 2 | ARG2 (228) |
| AI812030 | Thrombospondin 1 | THBS1 (79) |
| D32039 | Chondroitin sulphate | CSPG2 (22) |
| | Proteoglycan 2 | |
| | (versican) | |
| NM_001693 | ATPase | ATP6V1B2 |
| | H+transporting | |
| | protein (vATPase) | |

TABLE 2
Genes involved in defence against infection by
intracellular pathogens

| | | |
|---|---|---|
| NCBI Accession Number | Gene | Gene symbol |

Table 2A - Hub Genes

| | | |
|---|---|---|
| NM_002343 | Defensin α 1 | DEFA 1 |
| L_01639 | Chemokine (c-x-c) | CXCR4 |
| | receptor 4 | |
| M_87789 | Immunoglobulin heavy | IGHG3 |
| | constant γ3 | |
| NM_000570 | CD16 (IgG Fc | FCGR3A |
| | receptor, low | |
| | affinity IIIa) | |
| NM_002343 | Lactotransferrin | LTF |

Table 2B - Network Genes

| | | |
|---|---|---|
| NM_001925 | Defensin α 4 | DEFA 4 |
| NM_000419 | Integrin α 2B, CD41B | ITGA2B |
| M35999 | Integrin β 3 CD61 | ITGB3 |
| NM_000045 | Arginase 1 | ARG1 |
| U75667 | Arginase 2 | ARG2 |
| AI812030 | Thrombospondin 1 | THBS1 |

TABLE 3
Genes involved in oxidative stress response

| | | |
|---|---|---|
| NCBI Accession Number | Gene | Gene symbol |

Table 3A - Hub Genes

| | | |
|---|---|---|
| AI_133353 | Haemoglobin γ | HBG1 |

Table 3B - Network Genes

| | | |
|---|---|---|
| NM_000558 | Haemoglobin α 1 | HBA1 ( |
| S36219 | Prostaglandin- | PTGS1 |
| | endoperoxide | |
| | Synthase 1 | |
| NM_000963 | Prostaglandin- | PTGS2 |
| | endoperoxide | |
| | Synthase 2 | |

TABLE 4
Genes involved in apoptosis

| | | |
|---|---|---|
| NCBI Accession Number | Gene | Gene symbol |

Table 4A - Hub Genes

| | | |
|---|---|---|
| NM_004226 | Serine/threonine | STK17B |
| | kinase 17B | |

Table 4B - Network Genes

| | | |
|---|---|---|
| U13699 | Caspase 1 | CASP 1 |
| NM_012062 | Dynamin 1-like | DNM1L |
| NM_004657 | Phosphatidyl serine | SDPR |
| | binding prot. Serum | |
| | deprivation response | |

TABLE 5
Genes involved in antigen presentation and immunity

| | | |
|---|---|---|
| NCBI Accession Number | Gene | Gene symbol |

Table 5A - Hub Genes

| | | |
|---|---|---|
| BC005312 | Major | HLA-DRB4 |
| | Histocompatibility | |
| | Complex Class II DR | |
| | β4 | |

Table 5B - Network Genes

| | | |
|---|---|---|
| M16276 | Major | HLA-DQB1 |
| | Histocompatibility | |
| | Complex Class II | |
| | DQβ1 | |
| M 87789 | Immunoglobulin heavy | IGG3 |
| | constant γ3 | |

NCBI accession numbers refer to the UniGene database, build no. 177, released 22 Dec. 2004.

It is clear from this data that significant differences in the expression of a number of genes can be seen in PBMC samples from patients with CFS and healthy controls. We have verified that the DNA microarray assay is valid by confirming the results by RT-PCR and western blot analyses. This is the first time that a reproducible biochemical lesion has been seen in patients with CFS. We propose that bioassays of the significantly over-expressed genes could be used as diagnostic biomarkers for CFS to aid in the differential diagnosis of the condition.

In order to confirm the relevance of the genes identified in the microarray experiments, Western blot and RT-PCR assays have been performed to analyse the expression of selected genes in samples from patients and controls different to those studied in the microarray analysis. The results verify that these genes may be used as potential biomarkers to support the clinical diagnosis of CFS and identify suitable candidates for treatment trials.

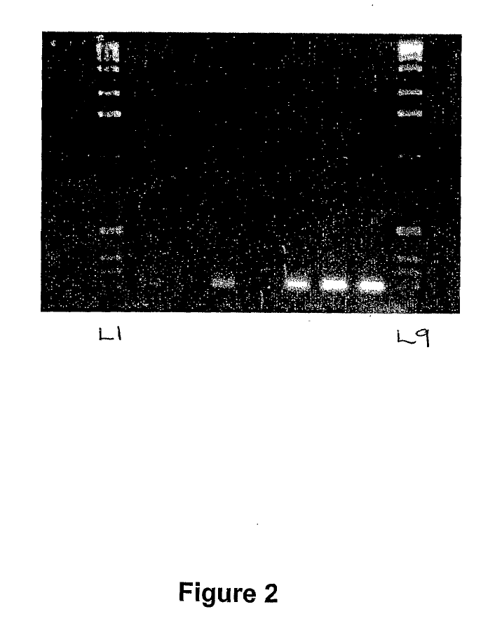

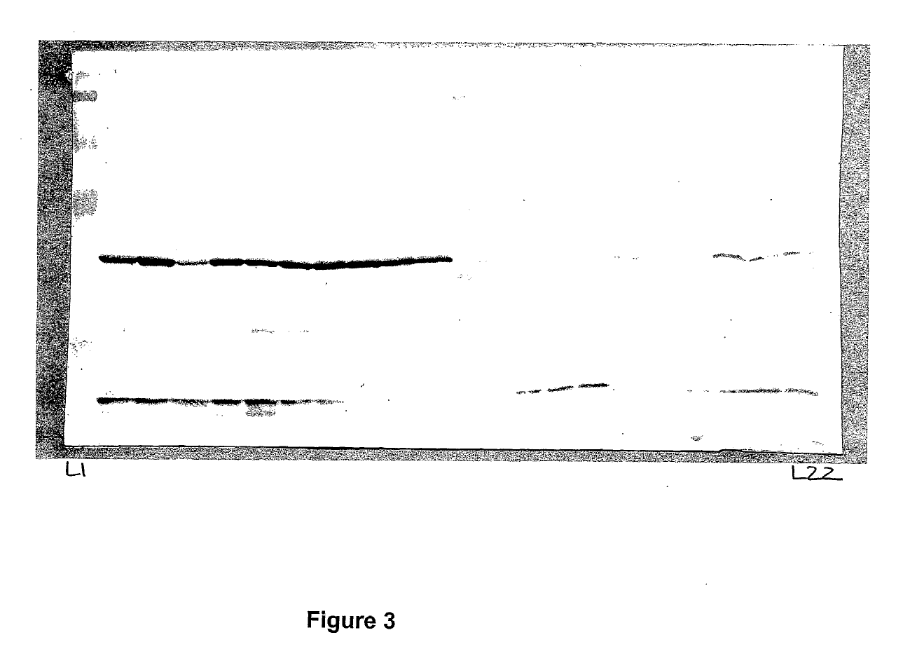

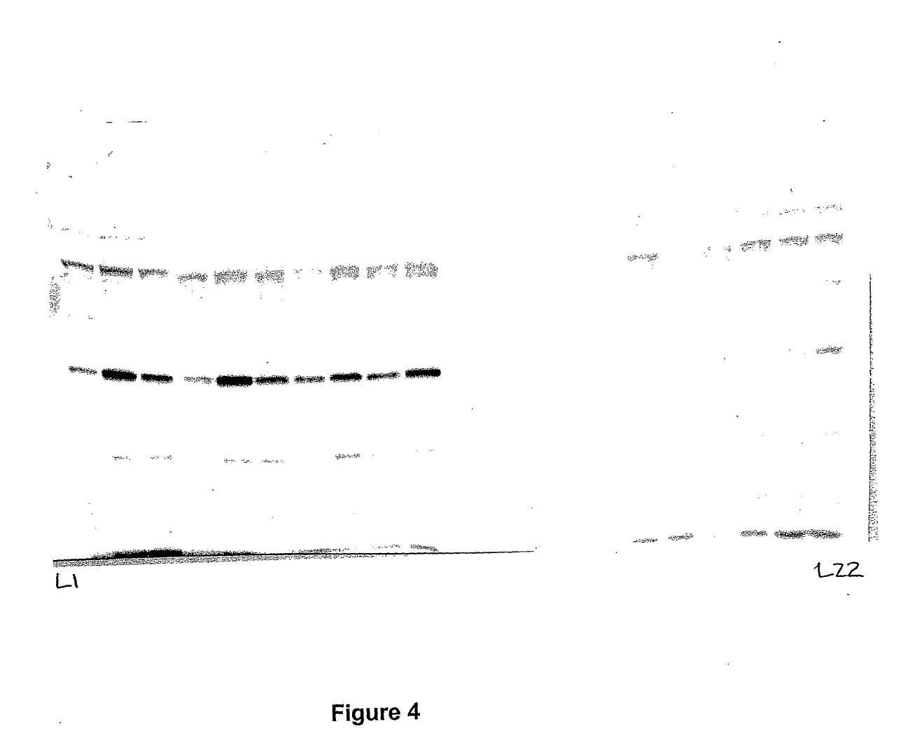

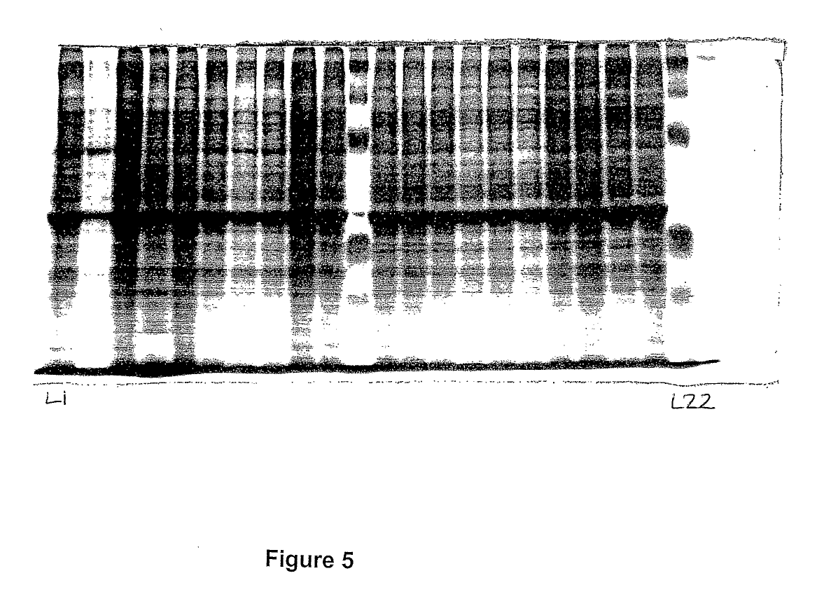

FIGS. 2 to 5 show upregulation of mRNA and/or proteins from various of these genes in PBMCs from patients suffering from CFS/ME, as compared to age- and sex-matched healthy controls. FIG. 2 shows RT-PCR analysis of Defensin alpha 1, FIGS. 3 and 4 show Western blot analysis of Defensin alpha 1 and Thrombospondin 1 respectively. In all cases, there is significant upregulation of the protein or mRNA in the disease group compared to the control group. FIG. 6 shows a duplicate of the SDS-PAGE gel used for the Western blot, stained with Coomassie blue. This confirms that the observed differences in protein levels between the disease and control groups are not due to unequal loading of total protein on the gels.

### Discussion

Previous reports have hypothesised that CFS is a form of channelopathy—a disorder of membrane ion channels^{9,11,13}. There are several reports in the literature which we believe strengthen the hypothesis that the vacuolar H+ ATPase plays a pathogenic role in CFS.

Local anaesthetics, which are known to act on ion channels, have an adverse effect on patients with CFS/ME. It has been demonstrated also, that in some patients with CFS/ME, there are morphological changes to the red blood cells¹⁹. Remarkably, a study by Nishiguchi et al²⁰, has demonstrated that the local anaesthetic lidocaine can induce reversible morphological transformation of human red blood cells and that this change is mediated by the activation of vacuolar H+ ATPase. In addition, Li et al^{21a} have shown that the gene is involved in iron binding in red blood cells.

The ion channel gene is a member of the vacuolar H+ ATPase proton transporting gene family^{21,22,23}. This family of genes is directly involved with the phosphocreatine-dependent glutamate uptake by synaptic vesicles²⁴. The gene is responsible for vesicle docking/exocytosis during neurotransmiter release²⁵ and is a major constituent of synaptic vesicles associated with intracellular membrane structures²⁶. We have demonstrated, using ¹H MRS that there is a perturbation of the choline/creatine balance in the CNS (Condon et al¹⁷, Chaudhuri et al⁴²). This finding has been corroborated by Puri et al¹⁸. As stated above, this type of gene is directly involved in the creatine pathways.

We have previously demonstrated that patients with CFS have low body potassium levels⁹. Bailey et al²⁷ have shown a relationship between potassium depletion and up-regulation of H+-ATPase.

As stated above, viruses have often been associated with CFS. Virus entry into cells may be mediated by H+ATPase^{28,29,30}. In addition to viral infection affecting neurotransmitter function⁷, there is a large body of evidence to show that the vacuolar H+-ATPase is also involved^{31,32,33,34,35,36,37,38,39}.

It is clear from the above data that significant differences in the expression of a number of genes can be seen in PBMC samples from patients with CFS and healthy controls. This is the first time that a reproducible biochemical lesion has been seen in patients with CFS. We propose that bioassays of the significantly over-expressed genes (below) could be used as diagnostic biomarkers for CFS to aid in the differential diagnosis of the condition.

### Materials and Methods

### Study Subjects

Patients with CFS were diagnosed with reference to the 1994 Fukuda definition. All seven patients were male, aged between 18 and 54 years (mean 3 6), and were not on medication. Healthy control subjects were male, aged between 22 and 58 years (mean 34). In addition to the 8 patients and seven control subjects used for the DNA chip assays, an additional fourteen patients and controls were used to confirm the chip assay results by RT-PCR and western blot assays. Informed consent and ethical approval were obtained.

### Isolation of Peripheral Blood Mononuclear Cells (PBMC) for Protein Purification

Venous blood samples were drawn from patients who fulfilled the Holmes and Fukuda criteria for ME/CFS, and from healthy individuals. The procedure for isolating PBMC was started immediately and finished within 2 h of sampling. EDTA treated whole blood was diluted 1:1 with phosphate buffered saline. Two volumes of blood were overlaid on one volume of Histopaque −1077 (Sigma Diagnostics) and centrifuged at 20° C. AT 500 g for 30 min. The PBMC interface was removed and washed twice with phosphate buffered saline and centrifuged. The pellets were resuspended in phosphate buffered saline, an aliquot removed and counted in red blood cell lysis buffer (155 Mm NH4CL, 10 Mm NaHCO3, pH7.4, 0.1 mM EDTA). The PBMC were centrifuged once more (20° C., 500 g, 10 min). The PBMC were then aliquoted into microtubes equivalent to 5×105 cells per tube, centrifuged and stored as dry pellets at −80° C.

### Western Blot Analysis

PBMC pellets were resuspended in sample reducing buffer (1 ml Glycerol, 0.5 ml β-mercaptoethanol, 3 ml 10% SDS, 1.25 ml 1M Tris-HCL Ph 6.7), boiled for 5 min. The lysates were loaded onto a 10% PAGE gel, each track equivalent to 2×10 5 PBMC. The PAGE gel was assessed for equal protein load by coomassie stain. The gel then electrophoretically transferred onto nitrocellulose PVDF membrane (Biorad) for 2 hours. The blots were blocked for non specific binding with 10% normal goat serum for 30 min, probed with a mouse Mab to human defensin1-3, (Hycult, Netherlands), mouse Mab to human thrombospondin (Sigma-Aldrich Inc) and a mouse Mab to Chondroitin Sulphate Proteoglycan (USBiological MA USA), at dilutions of 1/100, 1/1000, 1/1000 respectively in TBS 0.05% Tween 20 for 2 hours at RT. The protein was detected after subsequent incubation with alkaline phosphatase conjugate Goat anti Mouse IgG ( 1/1000 final dilution) (Jackson Immunoresearch Laboratories PA USA). The reactions were detected using SIGMA FAST BCIP/NBT).

### Development of the PCR Assay for the CFS Gene.

Oligonucleotide primers which span a specific epitope within the CFS gene were chosen and tested by RT-PCR. RNA from blood samples from patients with CFS and appropriate controls were RT-PCR amplified and PCR amplicons quantitated by gel documentation system software.

### RNA Isolation for RT-PCR

Total RNA was isolated from peripheral blood mononuclear cells (PBMC) using the Promega RNAgents Total RNA Isolation System.

Venous blood was collected in standard EDTA blood tubes and RNA purified using the method of Chomczynski and Sacchi. White blood cell pellets were homogenised by hand in an appropriate volume of denaturing solution (guanidinium thiocyanate, 4M; sodium citrate, 25 mM; N-laurolyl sarcosine, 0.5%; 2-mercaptoethanol, 0.1M, in distilled water adjusted to pH 7.0). To homogenate (1 ml), sodium acetate (100 μl, 2M, pH 4.0), citrate-buffered (0.1M, pH 4.3) phenol (1 ml) and chloroform: isoamyl alcohol (49:1, 200 μl) were sequentially added. The resulting mixture was treated in a vortex mixer (Fisons Scientific Equipment, Whirlimixer™) for 10 seconds then incubated on ice for 15 minutes. Samples were then centrifuged (12,000 g, 20 minutes, 4° C.) and the upper aqueous layer pippetted into to a fresh tube. After addition of ice cold isopropanol (1 ml), RNA precipitated from the mixture during a thirty minute period on dry ice. The mixtures were then centrifuged (12,000 g, 20 minutes, 4° C.) and the supernatant discarded. The pellet was dissolved in denaturing solution (300 μl) and transferred to a microcentrifuge tube (1.5 ml, Axygen). Ethanol (absolute, 600 μl) was added to each microcentrifuge tube, samples were incubated on dry ice for 30 minutes, and then centrifuged (11600 g, 20 minutes, 4° C.). The resulting pellet were then washed twice in ethanol (70% aqueous solution) then lyophylised (using a Hetosicc freeze-drier and a JAVAC high vacuum pump DD-75). The freeze-dried RNA was re-suspended in sterile distilled water (60 μl) and stored at −70° C. until required.

### Quantification and Examination of RNA

RNA solution (prepared as described above, 2 μl) was added to distilled water (98 μl) to produce a 1:50 dilution. The optical density of the sample was read at 260 nm and 280 nm using a 50 μl ultraviolet cuvet. The absorbance ratio 260/280 nm measured at these wavelengths indicates the purity of the RNA. The absolute concentration of RNA was estimated using the following equation:

Optical Density₂₆₀ₙₘ×40× dilution=RNA concentration (μg/ml)

To examine the quality, 5 μl of RNA was added to 5 μl of the gel marker Orange G (1% Orange G dye in 50% glycerol, 50% 2×TBE). This was heated to 70° C. for 3 minutes, then electrophoresed through a horizontal 1% agarose gel in 1×TBE (10×TBE=0.089M tris(hydroxymethyl)-methylamine, 0.089M boric acid, 0.025M disodium EDTA, pH 8.3). The gel was then stained for 30 minutes in ethidium bromide (0.5 mg/ml) in TBE, de-stained in water and visualised under medium wave (320 nm) ultraviolet light.

cDNA Synthesis

Two micrograms of RNA were added to 1 μl oligo (dT)₁₂₋₁₈ (500 μg/ml, Roche) and the volume made up to 11 μl with sterile distilled water. This was incubated (70° C., 10 minutes) to allow the oligo(dT) to bind to the poly-A tail of the RNA, and then chilled on ice. To the reaction mixture, 4 μl of 5× First Strand Buffer, 2 μl of 0.1M DTT, 1 μl of 10 mM dNTP mix (10 mM each dATP, dGTP, dCTP, dTTP; Amersham Pharmacia Biotech), 1 μl of sterile distilled water and 1 μl of Superscript II (Gibco-BRL® Life Technologies) were added. This was incubated first at 50° C. for 1 hr, then 70° C. for 15 minutes to inactivate the reaction. A negative cDNA control (2 μl of distilled water) was included in each cDNA synthesis to confirm that the reaction mix was not contaminated.

### Polymerase Chain Reaction

To 2 μl of cDNA, 10 μl of 10× magnesium-free buffer, 10 μl of 2.5 mM dNTPs (2.5 mM each dATP, dGTP, dCTP, dTTP), 6 μl MgCl₂ (25 mM) and 1 μl each of the appropriate 5′ and 3′ primers (0.5 μg/μl) were added. The volume was made up to 99.8 μl using sterile distilled water and 0.2 μl of Taq DNA polymerase (Promega) was added. The PCR reaction (35 cycles) was carried out on a Techne Genius thermocycler.

Primers used to amplify vATPase were 5′-ctc gtg acc tgt tac tgc tg-3′ and 5′-aag taa cca agt cca ctc ca-3′. Primers for Defensin 1 were 5′-caa gag ctg atg agg ttg ct-3′ and 5′-gaa ggt aca gga gta ata gc-3′.

Thirty microlitres of PCR product was added to 5 μl of orange G and electrophoresed through a horizontal 2-3% agarose gel in 1×TBE at 100 volts, until the dye front had migrated a minimum of 10 cm. On each gel, 3 μl of a 123 base pair DNA ladder in 5 μl of Orange G was included as a size marker for comparison with PCR product bands. The gel was then stained for 30 minutes in ethidium bromide (0.5 mg/ml) in TBE, destained in water and visualised under medium wave (320 nm) ultraviolet light.

Gene expression was analysed by measuring the band density for each amplicon. Band densities were measured using the Herolab EASY Plus computer automated image analysis system. True comparisons in gene expression between samples were enabled by comparing results of densitometry for the experimental genes against the housekeeping gene (abl—tyrosine kinase) band densities.

### REFERENCES

1. Chaudhuri A, Behan P O. Neurological dysfunction in chronic fatigue syndrome. J Chr Fatigue Synd 2000; 6(3/4):51-68
2. Fukuda K, Strauss S E, Hickie I, et al. The chronic fatigue syndrome: a comprehensive approach to its definition and study. Ann Intern Med 1994; 121:953-59
3. The National Task Force Report on CFS/ME (30 Sep. 1998).
4. Chaudhuri A, Behan W M H, Behan P O. Chronic fatigue syndrome. Proc R Coll Physicians Edinb 1998:28: 150-63
5. Chaudhuri A, Gow J, Behan P O. Systemic viral infections and chronic fatigue syndrome: current concept and recent advances. In: Abramsky O, Miller D (eds). Brain disease: Therapeutic strategies and repair. London: Martin Dunitz 2001 (in press)
6. de la Torre J C, Borrow P, Oldstone M B A. Viral persistence and disease: cytopathology in the absence of cytolysis. Br Med Bull 1991; 47: 838-51
7. Gies U, Bilzer T, Stitz L, Staiger J F. Disturbance of the cortical cholinergic innervation in Borna disease prior to encephalitis. Brain Pathology 1998; 8: 39-48
8. Kress M, Fickenscher H. Infection by human varicella-zoster virus confers norepinephrine sensitivity to sensory neurons from rat dorsal root ganglia. FASEB Journal 2001; 15:1037-43
9. Chaudhuri A, Watson W S, Behan P O. Arguments for a role of abnormal ionophore function in chronic fatigue syndrome. In: Yehuda S, Moltofsky D I (eds). Chronic fatigue syndrome. New York, Plenum Press 1997; 119-30
10. Goncealves P P, Meireles S M, Neves P, Vale M G P. Ca²⁺ sensitivity of synaptic vesicle dopamine, gamma-aminobutyric acid and glutamate transport system. Neurochemical research 2001; 26: 75-81
11. Gow J W, McGarry F, Behan W M H, Simpson K, Behan P O. Molecular analysis of cell membrane ion channel function in chronic fatigue syndrome (Abstract). International Meeting on Chronic Fatigue Syndrome, Dublin 1994.
12. Ackerman M J, Clapham D E. Ion channels-basic science and clinical diseases. N Engl J Med 1997; 336: 1575-86
13. Chaudhuri A, Watson W S, Pearn J, Behan P O. Symptoms of chronic fatigue syndrome are due to abnormal ion channel function. Medical Hypotheses 2000; 54: 59-63.
14. Pearn J. Chronic ciguatera: one organic cause of the chronic fatigue syndrome. J Chr Fatigue Synd 1996; 2: 29-34
15. Bidard J N, Viverberg H P M, Frelin C. Ciguatoxin is a novel type of Na⁺ channel toxin. J Biol Chem 1984; 259: 8353-57
16. Brinkmeier H, Aulkemeyer P, Wollinsky K H, Rudel R. An endogenous pentapeptide acting as a sodium channel blocker in inflammatory autoimmune disorders of the central nervous system. Nature Med 2000; 6: 808-11.
17. Condon B R, Chaudhuri A, Hadley D N, Brennan D, (2002), “Increased levels of choline containing compounds found in the basal ganglia of patients with chronic fatigue syndrome” in Proc Intl. Soc. Mag. Reson. Med. 10.
18. Puri B K, et al, (2002), “Relative increase in choline in the occipital cortex in chronic fatigue syndrome” Acta Psychiatr Scand 106(3):224-226.
19. Simpson L O. (1992) “The role of nondiscocytic erythrocytes in the pathogenesis of myalgic encephalomyelitis/chronic fatigue syndrome” In Hyde B M, Goldstein, Levine P (eds). The clinical and scientific basis of myalgic encephalomyelitis/chronic fatigue syndrome. The Nightingale Research Foundation, Canada. 597-605.
20. Nishiguchi E, Hamada N, Shindo J. (1995), “Lidocaine action and conformational changes in cytoskeletal protein network in human red blood cells” Eur J Pharmacol, 286(1):1-8.
21. Forgac, M. Structure and properties of the vacuolar (H⁺)-ATPases-. J. Biol. Chem. 274(19), 12951-12954 (1999).
21a. Li, C-Y., Watkins, J. A., Hamazaki, S., Altazan, J. D. & Glass, J. Iron binding, a new function for the reticulocyte endosome H+-ATPase. Biochemistry. 34(15), 5130-5136 (1995).
22. Stevens, T. H. & Forgac, M. Structure, function and regulation of the vacuolar (H⁺)-ATPase. Annu. Rev. Cell Dev. Biol 13, 779-808 (1997).
23. Nelson, N. & Harvey, W. R. Vacuolar and plasma membrane proton-adenosinetriphosphatases. Physiol. Rev. 79(2), 361-385 (1999).
24. Xu C J, Klunk W E, Kanfer J N, et al, (1996), Phosphocreatine-dependent glutamate uptake by synaptic vesicles” J Biological Chemistry, 271 (23): 13435-13440.
25. Siebert A, Lottspeich F, Nelson N, Betz H (1994). Purification of the synaptic vesicle-binding protein physophilin. Identification as 39-kDa subunit of the vacuolar H+-ATPase. J. Biological Chemistry. 269 (45): 28329-28334.
26. Brochier G, Morel N (1993). The same 15 kDA proteolipid subunit is a constituent of two different proteins in Torpedo, the acetycholine releasing protein and the vacuolar H+ATPase. Neurochem. International. 23 (6): 525-539.
27. Bailey, M., Capasso, G., Agulian, S., Giebisch, G. & Unwin, R. The relationship between distal tubular proton secretion and dietary potassium depletion: evidence for up-regulation of H+-ATPase. Nephrol. Dial. Transplant. 14(6), 1435-1440 (1999).
28. Hansen, J., Qing, K. & Srivastava, A. Adeno-associated virus type 2-medicated gene transfer: Altered endocytic processing enhances transduction efficiency in murine fibroblasts. J. Virol. 75(9), 4080-4090 (2001).
29. Suzuki, T. Yamayam M, Sekizawa, K., Hosada, M., Yamada, N., Ishizuka, S., nakayama, K., Yanai, M., Numazaki, Y., Sasaki, H. Bafilomycin A<inf>1</inf> inhibits rhinovirus infection in human airway epithelium: Effects on endosome and ICAM-1. Am. J. Physiol. Lung Cell. Mol. Physiol. 280(6 24-6), L1115-L1127 (2001).
30. Guinea, R. & Carrasco, L. Requirement for vacuolar proton-ATPase activity during entry influenza virus into cells. J. Virol. 69(4), 2306-2312 (1995).
31. Moriyama, Y. & Futai, M. Presence of 5-hydroxytryptamine (serotonin) transport coupled with vacuolar-type H+ATPase in neuroscretory granules from bovine posterior pituitary. J. Biol. Chem. 265(16), 9165-9169 (1990).
32. Wolosker, H., de Souza, D. O. & de Meis, L. Regulation of glutamate transport into synaptic vesicles by chloride and proton gradient. Am. Soc. Biochem. Mol. Bio. 271(20), 11726-11731 (1996).
33. Xu, C. J. et al. Phosphocreatine-dependent glutamate uptake by synaptic vesicles. A comparison with atp-dependent glutamate uptake. J. Bio. Chem. 271(23), 13435-13440 (1996).
34. Moriyama, Y., Tsai, H. L. & Futai, M. Energy-dependent accumulation of neuron blockers causes selective inhibition of neurotransmitter uptake by brain synaptic vesicles. Arch. Biochem. Biophys. 305(2), 278-281 (1993).
35. Moriyama Y. & Futai, M. Presence of 5-hydroxytryptamine (serotonin) transport coupled with vacuolar-type H(+)-ATPase in neurosecretory granules from bovine posterior pituitary. J. Biol. Chem. 265(16), 9165-9169 (1990).
36. Wolosker, H., de Souza, D. O. & de Meis, L. Regulation of glutamate transport into synaptic vesicles by chloride and proton gradient. J. Biol. Chem. 271(20), 11726-11731 (1996).
37. Brochier, G. & Morel, N. The same 15 kDa proteolipid subunit is a constituent of two different proteins in torpedo, the acetylcholine releasing protein mediatophore and the vacuolar H+ATPase. Neurochem. Int. 23(6), 525-539 (1993).
38. Hayashi, M., Haga, M., Yatsushiro, S., Yamamoto, A. & Moriyama, Y. Vesicular monoamine transporter 1 is responsible for storage of 5-hydroxytryptamine in rat pinealocytes. J. Neurochem. 73(6), 2538-2545 (1999).
39. Terland, O. & Flatmark, T. Drug-induced parkinsonism: cinnarizine and flunarizine are potent uncouplers of the vacuolar H+-ATPase in catecholamine storage vesicles. Neuropharmacology 38(6), 879-882 (1999).
39. Miyamoto, J. K., Uezu, E., Jiang, P. J. & Miyamoto, A. T. H(+)-ATPase and transport of DOPAC, HVA, and 5-HIAA in monoamine neurons. Physiol. Behav. 53(1), 65-74 (1993).
40. Ramsay, A. M. & Rundle, A. Clinical and biochemical findings in ten patients with benign myalgic encephalomyelitis. Postgrad. Med. J. 55(654), 856-857 (1979).
41. Condon B R, Chaudhuri A, Condon B R, Gow J W, Hadley D N, Brennan D, “Proton magnetic resonance spectroscopy of basal ganglia in chronic fatigue syndrome” Neuroreport 14(2), 225-228 (2003)
42. Chaudhuri A, Gow J W Behan P O, “Chronic fatigue syndrome and systemic viral infections: current evidence and recent advances.” in “Brain Disease—Therapeutic Strategies and Repair” Abramsky O, Compston D A, Miller A, Said G. 2002 pp 127-135.
43. Vernon, S D, Unger, E R, Dimulescu, I M, Rajeevan, M and Reeves, W C. Utility of the blood for gene expression profiling and biomarker discovery in chronic fatigue syndrome. Disease Markers 18, 193-199 (2002).
44. Klimas N G, Salvato F R, Morgan R, Fletcher M A. Immunological abnormalities in chronic fatigue syndrome. J Clin Microbiol 1990; 28: 1403-10.
45. Lloyd A R, Wakefield D, Hickie I. Immunity and the pathophysiology of chronic fatigue syndrome. Ciba Found Symp 1993; 173: 176-87.
46. Barker E, Fujimara S F, Fadem M B, Landay A L, Levy J A. Immunological abnormalities associated with chronic fatigue syndrome. Clin Infect Dis 1994; 18 (Suppl 1): S136-41.
47. Hassan I S, Bannister B A, Akbar A, Weir W, Bofill M. A study of the immunology of the chronic fatigue syndrome: correlation of immunologic parameters to health dysfunction. Clin Immunol Immunopathol 1998; 87: 60-7.
48. Vollmer-Conna U, Lloyd A, Hickie I, Wakefield D. Chronic fatigue syndrome: an immunological perspective. Aust NZ Psychiatry 1998; 32: 523-7.

TABLE 6

| | |
|---|---|
| Experiment | SK1U133 |
| Normalisation | rma across replicates |
| Chip | A |
| RP method | RP3 |
| Comparison | Samples: P2 P3 P4 P5 P6 P7 P8 P9 vs Samples: C1 C2 C3 C5 C6 C7 C8 |
| Direction | Positive |

| 0 | #Probe-set ID | RPscore | FDR | FCrma | FCnom | “SOURCE” | “UniGene” | Gene Symbol |
|---|---|---|---|---|---|---|---|---|
| 1 | 213515_x_at | 113.76 | 0.00 | 2.59 | 4.77 | AI133353 | Hs.449631 | HBG1 |
| 2 | 205033_s_at | 116.09 | 0.00 | 2.99 | 6.02 | NM_004084 | Hs.511887 | DEFA1 |
| 3 | 202018_s_at | 123.51 | 0.00 | 3.32 | 7.17 | NM_002343 | Hs.437457 | LTF |
| 4 | 206494_s_at | 152.52 | 0.00 | 2.34 | 4.04 | NM_000419 | Hs.411312 | ITGA2B |
| 5 | 206207_at | 158.06 | 0.00 | 2.87 | 5.63 | NM_001828 | Hs.889 | CLC |
| 6 | 209201_x_at | 175.29 | 0.00 | 2.20 | 3.64 | L01639 | Hs.421986 | CXCR4 |
| 7 | 204419_x_at | 177.28 | 0.00 | 2.29 | 3.89 | NM_000184 | Hs.449631 | HBG1 |
| 8 | 204627_s_at | 178.80 | 0.00 | 2.27 | 3.83 | M35999 | Hs.87149 | ITGB3 |
| 9 | 211919_s_at | 197.85 | 0.00 | 2.13 | 3.46 | AF348491 | Hs.421986 | CXCR4 |
| 10 | 216956_s_at | 218.78 | 0.00 | 2.09 | 3.36 | AF098114 | Hs.411312 | ITGA2B |
| 11 | 206834_at | 252.23 | 0.00 | 2.09 | 3.36 | NM_000519 | Hs.36977 | HBD |
| 12 | 204848_x_at | 259.39 | 0.00 | 2.06 | 3.27 | NM_000559 | Hs.449631 | HBG1 |
| 13 | 217739_s_at | 276.48 | 0.00 | 1.98 | 3.07 | NM_005746 | Hs.293464 | PBEF1 |
| 14 | 206493_at | 296.80 | 0.00 | 1.95 | 3.00 | NM_000419 | Hs.411312 | ITGA2B |
| 15 | 205214_at | 321.79 | 0.00 | 1.90 | 2.87 | NM_004226 | Hs.88297 | STK17B |
| 16 | 219630_at | 326.85 | 0.00 | 1.98 | 3.06 | NM_005764 | — | — |
| 17 | 209369_at | 329.37 | 0.00 | 2.12 | 3.43 | M63310 | Hs.442733 | ANXA3 |
| 18 | 205844_at | 359.70 | 0.00 | 1.93 | 2.93 | NM_004666 | Hs.12114 | VNN1 |
| 19 | 201058_s_at | 361.58 | 0.00 | 1.91 | 2.89 | NM_006097 | Hs.433814 | MYL9 |
| 20 | 207808_s_at | 376.44 | 0.00 | 1.97 | 3.04 | NM_000313 | — | — |
| 21 | 211571_s_at | 380.12 | 0.00 | 1.80 | 2.63 | D32039 | Hs.434488 | CSPG2 |
| 22 | 202708_s_at | 390.36 | 0.00 | 1.76 | 2.53 | NM_003528 | Hs.2178 | HIST2H2BE |
| 23 | 207269_at | 411.76 | 0.00 | 2.28 | 3.88 | NM_001925 | Hs.2582 | DEFA4 |
| 24 | 211560_s_at | 411.96 | 0.00 | 1.86 | 2.76 | AF130113 | Hs.440455 | ALAS2 |
| 25 | 206116_s_at | 423.80 | 0.00 | 1.82 | 2.66 | NM_000366 | Hs.133892 | TPM1 |
| 26 | 212515_s_at | 434.47 | 0.00 | 1.77 | 2.56 | BG492602 | Hs.380774 | DDX3X |
| 27 | 221768_at | 445.41 | 0.00 | 1.76 | 2.52 | AV705803 | Hs.180610 | SFPQ |
| 28 | AFFX-HSAC07/X00351_5_at | 472.31 | 0.00 | 1.75 | 2.49 | | | |
| 29 | 206676_at | 474.88 | 0.00 | 2.36 | 4.08 | M33326 | Hs.41 | CEACAM8 |
| 30 | 214696_at | 479.74 | 0.00 | 1.73 | 2.45 | AF070569 | Hs.417157 | MGC14376 |
| 31 | 206110_at | 496.83 | 0.00 | 1.48 | 1.91 | NM_003536 | Hs.70937 | HIST1H3H |
| 32 | 216202_s_at | 498.80 | 0.00 | 1.72 | 2.43 | U15555 | Hs.59403 | SPTLC2 |
| 33 | 201120_s_at | 509.82 | 0.00 | 1.71 | 2.40 | AL547946 | Hs.90061 | PGRMC1 |
| 34 | 216248_s_at | 512.18 | 0.00 | 1.82 | 2.66 | S77154 | Hs.82120 | NR4A2 |
| 35 | 209555_s_at | 523.56 | 0.00 | 1.70 | 2.40 | M98399 | Hs.443120 | CD36 |
| 36 | 212531_at | 530.54 | 0.00 | 1.82 | 2.67 | NM_005564 | Hs.204238 | LCN2 |
| 37 | 206655_s_at | 546.61 | 0.00 | 1.66 | 2.30 | NM_000407 | Hs.512000 | GP1BB |
| 38 | 216035_x_at | 547.30 | 0.03 | 1.64 | 2.25 | AV721430 | Hs.214039 | TCF7L2 |
| 39 | 204621_s_at | 548.80 | 0.03 | 1.72 | 2.44 | AI935096 | Hs.82120 | NR4A2 |
| 40 | 209116_x_at | 550.34 | 0.03 | 1.66 | 2.29 | M25079 | Hs.155376 | HBB |
| 41 | 210254_at | 550.38 | 0.02 | 1.96 | 3.02 | L35848 | Hs.99960 | MS4A3 |
| 42 | 219090_at | 559.00 | 0.05 | 1.71 | 2.40 | NM_020689 | Hs.439909 | SLC24A3 |
| 43 | 208792_s_at | 581.81 | 0.05 | 1.66 | 2.30 | M25915 | Hs.436657 | CLU |
| 44 | 209728_at | 582.70 | 0.05 | 1.39 | 1.71 | BC005312 | — | HLA-DRB4 |
| 45 | 207414_s_at | 586.02 | 0.04 | 1.63 | 2.23 | NM_002570 | Hs.117874 | PCSK6 |
| 46 | 201904_s_at | 599.75 | 0.04 | 1.62 | 2.21 | BF031714 | Hs.147189 | CTDSPL |
| 47 | 204466_s_at | 602.30 | 0.04 | 1.65 | 2.27 | BG260394 | Hs.76930 | SNCA |
| 48 | 209458_x_at | 604.65 | 0.04 | 1.56 | 2.08 | AF105974 | Hs.398636 | HBA2 |
| 49 | 217022_s_at | 604.71 | 0.04 | 1.77 | 2.55 | S55735 | Hs.366 | MGC27165 |
| 50 | 201980_s_at | 611.64 | 0.04 | 1.45 | 1.84 | NM_012425 | Hs.351342 | RSU1 |
| 51 | 203231_s_at | 613.03 | 0.04 | 1.76 | 2.52 | AW235612 | Hs.434961 | ATXN1 |
| 52 | 211745_x_at | 614.72 | 0.04 | 1.55 | 2.05 | BC005931 | Hs.398636 | HBA2 |
| 53 | 201005_at | 615.11 | 0.04 | 1.55 | 2.06 | NM_001769 | Hs.387579 | CD9 |
| 54 | 201906_s_at | 616.17 | 0.04 | 1.64 | 2.25 | NM_005808 | Hs.147189 | CTDSPL |
| 55 | 210244_at | 631.82 | 0.04 | 1.91 | 2.89 | U19970 | Hs.51120 | CAMP |
| 56 | 215813_s_at | 636.74 | 0.04 | 1.66 | 2.29 | S36219 | Hs.88474 | PTGS1 |
| 57 | 209480_at | 639.79 | 0.04 | 1.32 | 1.57 | M16276 | Hs.409934 | HLA-DQB1 |
| 58 | 210425_x_at | 642.62 | 0.03 | 1.66 | 2.31 | AF164622 | — | — |
| 59 | 208622_s_at | 643.22 | 0.03 | 1.66 | 2.30 | AA670344 | Hs.403997 | VIL2 |
| 60 | 216109_at | 649.72 | 0.03 | 1.65 | 2.28 | AK025348 | Hs.435249 | THRAP2 |
| 61 | 201739_at | 650.89 | 0.05 | 1.66 | 2.29 | NM_005627 | Hs.296323 | SGK |
| 62 | 215612_at | 660.86 | 0.05 | 1.63 | 2.22 | AU147983 | Hs.475627 | — |
| 63 | 208791_at | 668.35 | 0.05 | 1.62 | 2.21 | M25915 | Hs.436657 | CLU |
| 64 | 203827_at | 678.92 | 0.05 | 1.62 | 2.21 | NM_017983 | Hs.9398 | WIPI49 |
| 65 | 214731_at | 678.92 | 0.05 | 1.64 | 2.24 | AB037854 | Hs.374649 | DKFZp547A023 |
| 66 | 214805_at | 679.06 | 0.05 | 1.61 | 2.17 | U79273 | Hs.129673 | EIF4A1 |
| 67 | 212225_at | 683.10 | 0.04 | 1.62 | 2.20 | AL516854 | Hs.150580 | SUI1 |
| 68 | 202784_s_at | 683.67 | 0.04 | 1.61 | 2.19 | NM_012343 | Hs.106620 | NNT |
| 69 | 217738_at | 684.75 | 0.04 | 1.67 | 2.32 | BF575514 | Hs.293464 | PBEF1 |
| 70 | 207802_at | 687.00 | 0.04 | 1.93 | 2.94 | NM_006061 | Hs.404466 | CRISP3 |
| 71 | 200665_s_at | 695.48 | 0.04 | 1.67 | 2.31 | NM_003118 | Hs.111779 | SPARC |
| 72 | 208703_s_at | 698.66 | 0.04 | 1.63 | 2.22 | BG427393 | Hs.279518 | APLP2 |
| 73 | 204018_x_at | 703.07 | 0.04 | 1.51 | 1.96 | NM_000558 | Hs.398636 | HBA2 |
| 74 | 209395_at | 725.60 | 0.04 | 1.72 | 2.44 | M80927 | Hs.382202 | CHI3L1 |
| 75 | 201694_s_at | 730.85 | 0.05 | 1.45 | 1.84 | NM_001964 | Hs.326035 | EGR1 |
| 76 | 222186_at | 732.39 | 0.05 | 1.60 | 2.15 | AL109684 | Hs.306329 | — |
| 77 | 208601_s_at | 732.79 | 0.05 | 1.45 | 1.85 | NM_030773 | Hs.303023 | TUBB1 |
| 78 | 215933_s_at | 735.00 | 0.05 | 1.62 | 2.20 | Z21533 | Hs.118651 | HHEX |
| 79 | 201108_s_at | 749.47 | 0.05 | 1.58 | 2.12 | BF055462 | Hs.164226 | THBS1 |
| 80 | 204622_x_at | 752.07 | 0.06 | 1.60 | 2.15 | NM_006186 | Hs.82120 | NR4A2 |
| 81 | 218711_s_at | 752.67 | 0.06 | 1.52 | 2.00 | NM_004657 | Hs.26530 | SDPR |
| 82 | 210190_at | 755.04 | 0.06 | 1.59 | 2.14 | AF071504 | Hs.118958 | STX11 |
| 83 | 209315_at | 764.08 | 0.07 | 1.56 | 2.08 | AW297143 | Hs.221040 | HBS1L |
| 84 | 206167_s_at | 776.83 | 0.11 | 1.58 | 2.12 | NM_001174 | Hs.250830 | ARHGAP6 |
| 85 | 212592_at | 779.14 | 0.12 | 1.68 | 2.34 | AV733266 | Hs.381568 | IGJ |
| 86 | 211676_s_at | 781.53 | 0.13 | 1.59 | 2.14 | AF056979 | Hs.180866 | IFNGR1 |
| 87 | 215646_s_at | 784.29 | 0.14 | 1.58 | 2.11 | R94644 | Hs.434488 | CSPG2 |
| 88 | 217811_at | 791.60 | 0.15 | 1.57 | 2.10 | NM_016275 | Hs.8148 | SELT |
| 89 | 209868_s_at | 792.96 | 0.15 | 1.58 | 2.11 | D28482 | Hs.241567 | RBMS1 |
| 90 | 211828_s_at | 793.05 | 0.14 | 1.57 | 2.10 | AF172268 | Hs.252550 | TNIK |
| 91 | 208078_s_at | 794.06 | 0.14 | 1.60 | 2.17 | NM_030751 | Hs.380991 | SNF1LK |
| 92 | 204351_at | 798.71 | 0.15 | 1.57 | 2.10 | NM_005980 | Hs.2962 | S100P |
| 93 | 210387_at | 800.12 | 0.15 | 1.49 | 1.92 | BC001131 | Hs.182137 | HIST1H2BG |
| 94 | 203574_at | 801.59 | 0.15 | 1.48 | 1.91 | NM_005384 | — | — |
| 95 | 204628_s_at | 810.96 | 0.16 | 1.56 | 2.07 | NM_000212 | Hs.87149 | ITGB3 |
| 96 | 211367_s_at | 812.31 | 0.16 | 1.56 | 2.07 | U13699 | Hs.2490 | CASP1 |
| 97 | 216218_s_at | 813.86 | 0.15 | 1.57 | 2.10 | AK023546 | Hs.54886 | PLCL2 |
| 98 | 209964_s_at | 818.86 | 0.15 | 1.58 | 2.11 | AF032105 | Hs.108447 | ATXN7 |
| 99 | 212671_s_at | 825.56 | 0.15 | 1.37 | 1.67 | BG397856 | Hs.387679 | HLA-DQA1 |
| 100 | 208632_at | 826.40 | 0.15 | 1.54 | 2.03 | AL578551 | Hs.528722 | RNF10 |
| 101 | 217232_x_at | 826.87 | 0.15 | 1.48 | 1.91 | AF059180 | — | — |
| 102 | 204875_s_at | 828.71 | 0.15 | 1.55 | 2.06 | NM_001500 | Hs.105435 | GMDS |
| 103 | 206715_at | 831.12 | 0.15 | 1.52 | 1.99 | NM_012252 | Hs.125962 | TFEC |
| 104 | 206698_at | 836.18 | 0.14 | 1.56 | 2.07 | NM_021083 | Hs.78919 | XK |
| 105 | 208097_s_at | 838.41 | 0.15 | 1.55 | 2.05 | NM_030755 | Hs.125221 | TXNDC |
| 106 | 213359_at | 843.56 | 0.16 | 1.58 | 2.12 | W74620 | Hs.438726 | HNRPD |
| 107 | 205557_at | 843.61 | 0.16 | 1.75 | 2.51 | NM_001725 | Hs.303523 | BPI |
| 108 | 210987_x_at | 844.12 | 0.16 | 1.57 | 2.10 | M19267 | Hs.133892 | TPM1 |
| 109 | 201082_s_at | 849.04 | 0.16 | 1.55 | 2.04 | NM_004082 | Hs.74617 | DCTN1 |
| 110 | 214469_at | 850.41 | 0.15 | 1.39 | 1.72 | NM_021052 | Hs.121017 | HIST1H2AE |
| 111 | 209301_at | 854.28 | 0.15 | 1.44 | 1.81 | M36532 | Hs.155097 | CA2 |
| 112 | 208860_s_at | 859.17 | 0.16 | 1.55 | 2.04 | U09820 | Hs.440734 | ATRX |
| 113 | 213831_at | 868.41 | 0.16 | 1.07 | 1.12 | X00452 | Hs.289095 /// | HLA-DQA2 /// |
| | | | | | | | Hs.387679 | HLA-DQA1 |
| 114 | 220496_at | 869.62 | 0.16 | 1.45 | 1.84 | NM_016509 | Hs.409794 | CLEC2 |
| 115 | 203680_at | 871.65 | 0.16 | 1.57 | 2.09 | NM_002736 | Hs.77439 | PRKAR2B |
| 116 | 204794_at | 876.91 | 0.16 | 1.51 | 1.96 | NM_004418 | Hs.1183 | DUSP2 |
| 117 | 39313_at | 879.33 | 0.16 | 1.52 | 2.00 | AB002342 | Hs.275999 | PRKWNK1 |
| 118 | 208442_s_at | 881.57 | 0.16 | 1.49 | 1.93 | NM_000051 | Hs.526394 | ATM |
| 119 | 203630_s_at | 887.12 | 0.17 | 1.34 | 1.62 | NM_006348 | Hs.239631 | COG5 |
| 120 | 216511_s_at | 891.66 | 0.17 | 1.53 | 2.02 | AJ270770 | Hs.214039 | TCF7L2 |
| 121 | 201020_at | 892.51 | 0.17 | 1.49 | 1.92 | NM_003405 | Hs.226755 | YWHAH |
| 122 | 207827_x_at | 892.95 | 0.16 | 1.53 | 2.01 | L36675 | Hs.76930 | SNCA |
| 123 | 222040_at | 893.13 | 0.16 | 1.55 | 2.04 | AI144007 | Hs.356721 | HNRPA1 |
| 124 | 205997_at | 893.26 | 0.16 | 1.55 | 2.04 | NM_021778 | Hs.528304 | ADAM28 |
| 125 | 212749_s_at | 893.86 | 0.16 | 1.53 | 2.02 | AI096477 | Hs.48297 | RCHY1 |
| 126 | 215223_s_at | 898.45 | 0.16 | 1.47 | 1.87 | W46388 | Hs.384944 | SOD2 |
| 127 | 204081_at | 898.67 | 0.16 | 1.53 | 2.02 | NM_006176 | Hs.232004 | NRGN |
| 128 | 221860_at | 901.45 | 0.16 | 1.54 | 2.03 | AL044078 | Hs.446623 | HNRPL |
| 129 | 218486_at | 905.14 | 0.16 | 1.51 | 1.97 | AA149594 | Hs.12229 | TIEG2 |
| 130 | 201329_s_at | 908.48 | 0.15 | 1.54 | 2.03 | NM_005239 | Hs.292477 | ETS2 |
| 131 | 206343_s_at | 909.24 | 0.15 | 1.50 | 1.94 | NM_013959 | Hs.172816 | NRG1 |
| 132 | 212224_at | 909.91 | 0.15 | 1.55 | 2.05 | NM_000689 | Hs.76392 | ALDH1A1 |
| 133 | 213674_x_at | 915.53 | 0.16 | 1.55 | 2.04 | AI858004 | Hs.439852 | IGHM |
| 134 | 206283_s_at | 921.65 | 0.17 | 1.44 | 1.82 | NM_003189 | Hs.73828 | TAL1 |
| 135 | 211699_x_at | 923.85 | 0.17 | 1.42 | 1.77 | AF349571 | Hs.398636 | HBA2 |
| 136 | 214677_x_at | 927.71 | 0.18 | 1.53 | 2.01 | X57812 | Hs.405944 | IGLC2 |
| 137 | 215794_x_at | 932.98 | 0.18 | 1.52 | 1.99 | AC006144 | — | — |
| 138 | 221989_at | 936.30 | 0.18 | 1.55 | 2.05 | AW057781 | Hs.401929 | RPL10 |
| 139 | 202545_at | 939.10 | 0.19 | 1.51 | 1.97 | NM_006254 | Hs.155342 | PRKCD |
| 140 | 219079_at | 939.61 | 0.19 | 1.53 | 2.01 | NM_016230 | Hs.5741 | NCB5OR |
| 141 | 208180_s_at | 942.37 | 0.19 | 1.28 | 1.49 | NM_003543 | — | — |
| 142 | 222142_at | 943.96 | 0.19 | 1.51 | 1.96 | AK024212 | Hs.386952 | CYLD |
| 143 | 208884_s_at | 945.94 | 0.20 | 1.53 | 2.00 | AF006010 | Hs.94262 | EDD |
| 144 | 204993_at | 948.67 | 0.19 | 1.48 | 1.91 | NM_002073 | Hs.437081 | GNAZ |
| 145 | 213338_at | 949.40 | 0.19 | 1.47 | 1.89 | BF062629 | Hs.35861 | RIS1 |
| 146 | 204187_at | 951.00 | 0.20 | 1.44 | 1.82 | NM_006877 | Hs.1435 | GMPR |
| 147 | 221039_s_at | 959.09 | 0.22 | 1.51 | 1.97 | NM_018482 | Hs.386779 | DDEF1 |
| 148 | 217762_s_at | 961.00 | 0.23 | 1.49 | 1.92 | BE789881 | Hs.223025 | RAB31 |
| 149 | 209729_at | 967.16 | 0.23 | 1.51 | 1.98 | BC001782 | Hs.322852 | GAS2L1 |
| 150 | 202643_s_at | 968.93 | 0.23 | 1.39 | 1.71 | AI738896 | Hs.211600 | TNFAIP3 |
| 151 | 208931_s_at | 971.16 | 0.24 | 1.50 | 1.95 | AF147209 | Hs.256583 | ILF3 |
| 152 | 217414_x_at | 972.26 | 0.24 | 1.44 | 1.81 | V00489 | Hs.398636 | HBA2 |
| 153 | 201179_s_at | 975.44 | 0.24 | 1.51 | 1.97 | J03005 | Hs.73799 | GNAI3 |
| 154 | 201123_s_at | 976.94 | 0.24 | 1.51 | 1.97 | NM_001970 | Hs.310621 | EIF5A |
| 155 | 200628_s_at | 978.77 | 0.24 | 1.45 | 1.84 | M61715 | Hs.82030 | WARS |
| 156 | 220189_s_at | 983.96 | 0.24 | 1.50 | 1.95 | NM_014275 | Hs.437277 | MGAT4B |
| 157 | 212077_at | 985.45 | 0.24 | 1.39 | 1.72 | AL583520 | Hs.443811 | CALD1 |
| 158 | AFFX- | 986.61 | 0.23 | 1.51 | 1.97 | | | |
| | HSAC07/X00351_M_at | | | | | | | |
| 159 | 207275_s_at | 989.13 | 0.24 | 1.49 | 1.92 | NM_001995 | Hs.406678 | ACSL1 |
| 160 | 221556_at | 989.40 | 0.24 | 1.46 | 1.86 | BF792631 | Hs.22116 | CDC14B |
| 161 | 208010_s_at | 992.61 | 0.24 | 1.52 | 1.98 | NM_012411 | Hs.87860 | PTPN22 |
| 162 | 205884_at | 996.27 | 0.25 | 1.52 | 1.98 | NM_000885 | Hs.528404 | ITGA4 |
| 163 | 212813_at | 998.28 | 0.25 | 1.46 | 1.86 | AA149644 | Hs.419149 | JAM3 |
| 164 | 216037_x_at | 1002.76 | 0.24 | 1.43 | 1.79 | AA664011 | Hs.214039 | TCF7L2 |
| 165 | 211430_s_at | 1008.61 | 0.26 | 1.60 | 2.17 | M87789 | Hs.413826 | IGHG1 |
| 166 | 217821_s_at | 1008.83 | 0.26 | 1.51 | 1.96 | AF118023 | Hs.164526 | WBP11 |
| 167 | 219269_at | 1012.63 | 0.26 | 1.51 | 1.97 | NM_024567 | Hs.458684 | FLJ21616 |
| 168 | 211985_s_at | 1016.13 | 0.27 | 1.51 | 1.97 | AI653730 | Hs.282410 | CALM1 |
| 169 | 214974_x_at | 1017.48 | 0.27 | 1.41 | 1.75 | AK026546 | Hs.89714 | CXCL5 |
| 170 | 211026_s_at | 1023.05 | 0.28 | 1.54 | 2.02 | BC006230 | Hs.409826 | MGLL |
| 171 | 216905_s_at | 1032.03 | 0.29 | 1.46 | 1.86 | U20428 | Hs.56937 | ST14 |
| 172 | 217826_s_at | 1032.22 | 0.29 | 1.39 | 1.73 | NM_016021 | Hs.184325 | UBE2J1 |
| 173 | 203819_s_at | 1037.62 | 0.29 | 1.46 | 1.85 | AU160004 | Hs.79440 | IMP-3 |
| 174 | 211998_at | 1037.91 | 0.29 | 1.49 | 1.92 | AW138159 | Hs.180877 | H3F3B |
| 175 | 218055_s_at | 1040.13 | 0.29 | 1.47 | 1.89 | NM_018268 | Hs.16470 | WDR41 |
| 176 | 202768_at | 1042.14 | 0.30 | 1.29 | 1.52 | NM_006732 | Hs.75678 | FOSB |
| 177 | 204440_at | 1042.60 | 0.29 | 1.38 | 1.70 | NM_004233 | Hs.79197 | CD83 |
| 178 | 213524_s_at | 1045.57 | 0.29 | 1.13 | 1.23 | NM_015714 | Hs.432132 | G0S2 |
| 179 | 209839_at | 1051.62 | 0.31 | 1.44 | 1.82 | AL136712 | Hs.327347 | DNM3 |
| 180 | 220532_s_at | 1055.32 | 0.31 | 1.12 | 1.20 | NM_014020 | Hs.190161 | LR8 |
| 181 | 201075_s_at | 1057.31 | 0.31 | 1.48 | 1.90 | NM_003074 | Hs.162086 | SMARCC1 |
| 182 | 215071_s_at | 1067.30 | 0.31 | 1.43 | 1.79 | AL353759 | Hs.28777 | HIST1H2AC |
| 183 | 204748_at | 1073.73 | 0.32 | 1.43 | 1.80 | NM_000963 | Hs.196384 | PTGS2 |
| 184 | 208782_at | 1075.69 | 0.32 | 1.41 | 1.75 | BC000055 | Hs.433622 | FSTL1 |
| 185 | 206857_s_at | 1076.17 | 0.32 | 1.36 | 1.66 | NM_004116 | Hs.407482 | FKBP1B |
| 186 | 216621_at | 1076.93 | 0.32 | 1.47 | 1.89 | AL050032 | — | — |
| 187 | 214438_at | 1076.99 | 0.32 | 1.45 | 1.85 | M60721 | Hs.74870 | HLX1 |
| 188 | 207983_s_at | 1078.35 | 0.31 | 1.50 | 1.95 | NM_006603 | Hs.8217 | STAG2 |
| 189 | 201222_s_at | 1079.04 | 0.31 | 1.49 | 1.92 | AL527365 | Hs.159087 | RAD23B |
| 190 | 206036_s_at | 1083.29 | 0.31 | 1.48 | 1.90 | NM_002908 | Hs.44313 | REL |
| 191 | 201015_s_at | 1087.36 | 0.31 | 1.56 | 2.07 | NM_021991 | Hs.2340 | JUP |
| 192 | 202006_at | 1088.57 | 0.31 | 1.46 | 1.85 | NM_002835 | Hs.62 | PTPN12 |
| 193 | 206548_at | 1088.69 | 0.31 | 1.38 | 1.70 | NM_024880 | — | FLJ23556 |
| 194 | 202840_at | 1093.73 | 0.31 | 1.46 | 1.85 | NM_003487 | Hs.402752 | TAF15 |
| 195 | 201225_s_at | 1094.11 | 0.31 | 1.48 | 1.90 | NM_005839 | Hs.18192 | SRRM1 |
| 196 | 205933_at | 1094.99 | 0.32 | 1.47 | 1.89 | NM_015559 | Hs.201369 | SETBP1 |
| 197 | 202203_s_at | 1098.14 | 0.32 | 1.30 | 1.53 | NM_001144 | Hs.295137 | AMFR |
| 198 | 202241_at | 1098.22 | 0.32 | 1.33 | 1.59 | NM_025195 | Hs.444947 | TRIB1 |
| 199 | 202539_s_at | 1099.98 | 0.33 | 1.49 | 1.93 | AL518627 | Hs.11899 | HMGCR |
| 200 | 214875_x_at | 1105.10 | 0.33 | 1.47 | 1.87 | AW001847 | Hs.279518 | APLP2 |
| 201 | 214475_x_at | 1111.61 | 0.35 | 1.48 | 1.90 | AF127764 | Hs.439343 | CAPN3 |
| 202 | 213537_at | 1113.02 | 0.35 | 1.52 | 1.99 | AI128225 | Hs.914 | HLA-DPA1 |
| 203 | 204006_s_at | 1119.79 | 0.37 | 1.42 | 1.77 | NM_000570 | — | FCGR3A |
| 204 | 205128_x_at | 1120.16 | 0.37 | 1.47 | 1.87 | NM_000962 | Hs.88474 | PTGS1 |
| 205 | 210986_s_at | 1121.08 | 0.37 | 1.46 | 1.86 | Z24727 | Hs.133892 | TPM1 |
| 206 | 221942_s_at | 1121.44 | 0.37 | 1.42 | 1.78 | AI719730 | Hs.433488 | GUCY1A3 |
| 207 | 210222_s_at | 1127.24 | 0.37 | 1.45 | 1.84 | BC000314 | Hs.99947 | RTN1 |
| 208 | 209138_x_at | 1128.73 | 0.37 | 1.46 | 1.86 | M87790 | Hs.458262 | IGLC2 |
| 209 | 206515_at | 1129.17 | 0.37 | 1.42 | 1.78 | NM_000896 | Hs.106242 | CYP4F3 |
| 210 | 201681_s_at | 1129.81 | 0.37 | 1.49 | 1.92 | AB011155 | Hs.500245 | DLG5 |
| 211 | 203126_at | 1135.42 | 0.37 | 1.43 | 1.80 | NM_014214 | Hs.5753 | IMPA2 |
| 212 | 220832_at | 1136.62 | 0.37 | 1.50 | 1.94 | NM_016610 | Hs.272410 | TLR8 |
| 213 | 202007_at | 1137.65 | 0.38 | 1.48 | 1.91 | BF940043 | Hs.356624 | NID |
| 214 | 217591_at | 1138.78 | 0.38 | 1.44 | 1.82 | BF725121 | — | — |
| 215 | 217781_s_at | 1138.96 | 0.38 | 1.46 | 1.87 | NM_022473 | Hs.188199 | SH3BP3 |
| 216 | 208983_s_at | 1142.28 | 0.38 | 1.46 | 1.87 | M37780 | Hs.78146 | PECAM1 |
| 217 | 202944_at | 1149.07 | 0.41 | 1.43 | 1.80 | NM_000262 | Hs.75372 | NAGA |
| 218 | 207079_s_at | 1149.61 | 0.41 | 1.49 | 1.93 | NM_005466 | Hs.167738 | MED6 |
| 219 | 211696_x_at | 1150.69 | 0.42 | 1.37 | 1.68 | AF349114 | Hs.155376 | HBB |
| 220 | 202934_at | 1152.15 | 0.42 | 1.46 | 1.85 | AI761561 | Hs.406266 | HK2 |
| 221 | 213703_at | 1153.69 | 0.43 | 1.50 | 1.94 | W95043 | Hs.446532 | LOC150759 |
| 222 | 206618_at | 1156.05 | 0.44 | 1.50 | 1.95 | NM_003855 | Hs.159301 | IL18R1 |
| 223 | 202583_s_at | 1159.24 | 0.46 | 1.47 | 1.88 | NM_005493 | Hs.306242 | RANBP9 |
| 224 | 202861_at | 1162.81 | 0.46 | 1.46 | 1.86 | NM_002616 | Hs.445534 | PER1 |
| 225 | 214102_at | 1167.57 | 0.48 | 1.46 | 1.86 | AK023737 | — | — |
| 226 | 203748_x_at | 1168.88 | 0.48 | 1.47 | 1.87 | NM_016839 | Hs.241567 | RBMS1 |
| 227 | 204362_at | 1169.74 | 0.48 | 1.44 | 1.82 | NM_003930 | Hs.410745 | SCAP2 |
| 228 | 207610_s_at | 1173.18 | 0.49 | 1.46 | 1.87 | NM_013447 | Hs.137354 | EMR2 |
| 229 | 211822_s_at | 1173.74 | 0.49 | 1.45 | 1.85 | AF229061 | Hs.104305 | NALP1 |
| 230 | 201118_at | 1180.99 | 0.50 | 1.46 | 1.87 | NM_002631 | Hs.392837 | PGD |
| 231 | 212573_at | 1182.29 | 0.49 | 1.44 | 1.82 | AF131747 | Hs.167115 | KIAA0830 |
| 232 | 220711_at | 1183.01 | 0.49 | 1.43 | 1.79 | NM_024978 | — | — |
| 233 | 209795_at | 1184.57 | 0.49 | 1.40 | 1.74 | L07555 | Hs.82401 | CD69 |
| 234 | 221239_s_at | 1187.11 | 0.49 | 1.47 | 1.88 | NM_030764 | Hs.194976 | SPAP1 |
| 235 | 203127_s_at | 1189.65 | 0.50 | 1.44 | 1.81 | BC005123 | Hs.59403 | SPTLC2 |
| 236 | 204141_at | 1191.19 | 0.50 | 1.22 | 1.39 | NM_001069 | Hs.512712 | TUBB |
| 237 | 219892_at | 1194.44 | 0.51 | 1.43 | 1.81 | NM_023003 | Hs.151155 | TM6SF1 |
| 238 | 221523_s_at | 1195.49 | 0.51 | 1.46 | 1.86 | AL138717 | Hs.238679 | RRAGD |
| 239 | 210858_x_at | 1200.47 | 0.52 | 1.40 | 1.73 | U26455 | Hs.526394 | ATM |
| 240 | 204069_at | 1201.66 | 0.53 | 1.30 | 1.54 | NM_002398 | Hs.170177 | MEIS1 |
| 241 | 207332_s_at | 1202.05 | 0.52 | 1.46 | 1.86 | NM_003234 | Hs.185726 | TFRC |
| 242 | 209091_s_at | 1204.16 | 0.52 | 1.44 | 1.81 | AF263293 | Hs.136309 | SH3GLB1 |
| 243 | 200605_s_at | 1204.84 | 0.52 | 1.46 | 1.87 | NM_002734 | Hs.280342 | PRKAR1A |
| 244 | 201125_s_at | 1206.58 | 0.52 | 1.41 | 1.75 | NM_002213 | Hs.149846 | ITGB5 |
| 245 | 214765_s_at | 1208.50 | 0.52 | 1.45 | 1.83 | AK024677 | Hs.264330 | ASAHL |
| 246 | 207266_x_at | 1210.67 | 0.52 | 1.46 | 1.85 | NM_016837 | Hs.241567 | RBMS1 |
| 247 | 215786_at | 1213.10 | 0.53 | 1.46 | 1.87 | AK022170 | Hs.432941 | — |
| 248 | 220306_at | 1216.50 | 0.52 | 1.37 | 1.68 | NM_017709 | Hs.356216 | FAM46C |
| 249 | 208633_s_at | 1228.49 | 0.55 | 1.46 | 1.85 | W61052 | Hs.372463 | MACF1 |
| 250 | 205547_s_at | 1232.07 | 0.56 | 1.37 | 1.68 | NM_003186 | Hs.410977 | TAGLN |
| 251 | 209460_at | 1232.41 | 0.56 | 1.45 | 1.84 | AF237813 | Hs.1588 | ABAT |
| 252 | 203585_at | 1242.84 | 0.58 | 1.45 | 1.84 | NM_007150 | Hs.16622 | ZNF185 |
| 253 | 209043_at | 1244.35 | 0.59 | 1.43 | 1.80 | AF033026 | Hs.3833 | PAPSS1 |
| 254 | 201629_s_at | 1250.18 | 0.60 | 1.43 | 1.79 | BE872974 | Hs.130873 | ACP1 |
| 255 | 205442_at | 1253.38 | 0.60 | 1.36 | 1.65 | NM_021647 | Hs.178121 | MFAP3L |
| 256 | 210543_s_at | 1257.97 | 0.61 | 1.45 | 1.84 | U34994 | Hs.415749 | PRKDC |
| 257 | 221428_s_at | 1258.97 | 0.61 | 1.46 | 1.87 | NM_030921 | Hs.438970 | TBL1XR1 |
| 258 | 220094_s_at | 1259.66 | 0.61 | 1.33 | 1.60 | NM_022102 | Hs.214043 | C6orf79 |
| 259 | 200648_s_at | 1263.21 | 0.61 | 1.42 | 1.78 | NM_002065 | Hs.442669 | GLUL |
| 260 | 206204_at | 1263.28 | 0.61 | 1.30 | 1.54 | NM_004490 | Hs.411881 | GRB14 |
| 261 | 208925_at | 1270.79 | 0.63 | 1.48 | 1.89 | AF161522 | Hs.107393 | C3orf4 |
| 262 | 202275_at | 1270.96 | 0.63 | 1.21 | 1.36 | NM_000402 | Hs.80206 | G6PD |
| 263 | 204361_s_at | 1271.53 | 0.63 | 1.42 | 1.79 | AB014486 | Hs.410745 | SCAP2 |
| 264 | 209156_s_at | 1284.11 | 0.66 | 1.44 | 1.81 | AY029208 | Hs.420269 | COL6A2 |
| 265 | 222043_at | 1285.56 | 0.66 | 1.41 | 1.76 | AI982754 | Hs.436657 | CLU |
| 266 | 210982_s_at | 1287.56 | 0.67 | 1.43 | 1.80 | M60333 | Hs.409805 | HLA-DRA |
| 267 | 218319_at | 1287.89 | 0.67 | 1.43 | 1.80 | NM_020651 | Hs.7886 | PELI1 |
| 268 | 206641_at | 1291.70 | 0.67 | 1.52 | 1.98 | NM_001192 | Hs.2556 | TNFRSF17 |
| 269 | 215898_at | 1292.15 | 0.67 | 1.43 | 1.80 | AK021879 | Hs.435270 | KIAA0998 |
| 270 | 210379_s_at | 1295.24 | 0.67 | 1.43 | 1.80 | AF162666 | Hs.369280 | TLK1 |
| 271 | 203946_s_at | 1300.70 | 0.69 | 1.26 | 1.45 | U75667 | Hs.172851 | ARG2 |
| 272 | 212561_at | 1300.81 | 0.68 | 1.43 | 1.79 | AA349595 | Hs.26797 | RAB6IP1 |
| 273 | 208022_s_at | 1305.54 | 0.69 | 1.39 | 1.72 | NM_003671 | Hs.22116 | CDC14B |
| 274 | 201386_s_at | 1306.58 | 0.69 | 1.46 | 1.85 | AF279891 | Hs.5683 | DHX15 |
| 275 | 212036_s_at | 1312.00 | 0.72 | 1.44 | 1.81 | AW152664 | Hs.409965 | PNN |
| 276 | 200008_s_at | 1315.42 | 0.74 | 1.43 | 1.79 | D13988 | Hs.56845 | GDI2 |
| 277 | 212443_at | 1316.21 | 0.74 | 1.41 | 1.76 | AB011112 | Hs.437043 | KIAA0540 |
| 278 | 217523_at | 1317.38 | 0.74 | 1.45 | 1.83 | AV700298 | Hs.306278 | CD44 |
| 279 | 212337_at | 1317.38 | 0.74 | 1.43 | 1.80 | AI687738 | — | TI-227H |
| 280 | 212768_s_at | 1318.95 | 0.75 | 1.75 | 2.50 | AL390736 | Hs.273321 | OLFM4 |
| 281 | 208750_s_at | 1320.26 | 0.75 | 1.43 | 1.80 | AA580004 | Hs.286221 | ARF1 |
| 282 | 206177_s_at | 1324.86 | 0.76 | 1.59 | 2.13 | NM_000045 | Hs.440934 | ARG1 |
| 283 | 200730_s_at | 1325.35 | 0.76 | 1.29 | 1.52 | BF576710 | Hs.227777 | PTP4A1 |
| 284 | 202555_s_at | 1330.16 | 0.79 | 1.41 | 1.75 | NM_005965 | Hs.386078 | MYLK |
| 285 | 202466_at | 1330.57 | 0.79 | 1.43 | 1.80 | NM_006999 | Hs.39488 | POLS |
| 286 | 215992_s_at | 1331.28 | 0.79 | 1.42 | 1.77 | AL117397 | Hs.373588 | RAPGEF2 |
| 287 | 218033_s_at | 1337.07 | 0.81 | 1.33 | 1.59 | NM_003498 | Hs.76691 | SNN |
| 288 | 200946_x_at | 1338.29 | 0.82 | 1.43 | 1.80 | AI339331 | Hs.355697 | GLUD1 |
| 289 | 206571_s_at | 1345.69 | 0.85 | 1.39 | 1.72 | NM_004834 | Hs.6631 | MAP4K4 |
| 290 | 217202_s_at | 1355.53 | 0.89 | 1.40 | 1.74 | U08626 | Hs.442669 | GLUL |
| 291 | 210075_at | 1358.59 | 0.89 | 1.36 | 1.66 | AF151074 | Hs.331308 | LOC51257 |
| 292 | 201110_s_at | 1359.40 | 0.90 | 1.37 | 1.69 | NM_003246 | Hs.164226 | THBS1 |
| 293 | 215379_x_at | 1359.47 | 0.89 | 1.42 | 1.78 | AV698647 | Hs.405944 | IGLC2 |
| 294 | 221833_at | 1359.74 | 0.89 | 1.39 | 1.73 | AI971258 | Hs.301872 | LONP |
| 295 | 203357_s_at | 1360.30 | 0.89 | 1.42 | 1.79 | NM_014296 | Hs.7145 | CAPN7 |
| 296 | 220148_at | 1360.78 | 0.89 | 1.30 | 1.54 | NM_022568 | Hs.18443 | ALDH8A1 |
| 297 | 202940_at | 1369.07 | 0.91 | 1.42 | 1.77 | NM_014823 | Hs.275999 | PRKWNK1 |
| 298 | 207945_s_at | 1372.25 | 0.92 | 1.41 | 1.77 | NM_001893 | Hs.378918 | CSNK1D |
| 299 | 205552_s_at | 1374.66 | 0.93 | 1.40 | 1.73 | NM_002534 | Hs.442936 | OAS1 |
| 300 | 211824_x_at | 1376.06 | 0.93 | 1.41 | 1.75 | AF229062 | Hs.104305 | NALP1 |
| 301 | 201401_s_at | 1376.53 | 0.93 | 1.40 | 1.74 | M80776 | Hs.83636 | ADRBK1 |
| 302 | 203992_s_at | 1379.33 | 0.94 | 1.43 | 1.79 | AF000992 | — | UTX |
| 303 | 209930_s_at | 1381.61 | 0.94 | 1.38 | 1.70 | L13974 | Hs.75643 | NFE2 |
| 304 | 214366_s_at | 1394.78 | 1.00 | 1.42 | 1.77 | AA995910 | Hs.89499 | ALOX5 |
| 305 | 208501_at | 1395.46 | 1.00 | 1.43 | 1.80 | NM_004188 | — | — |
| 306 | 205730_s_at | 1396.55 | 1.00 | 1.37 | 1.67 | NM_014945 | Hs.282566 | ABLIM3 |
| 307 | 201313_at | 1396.63 | 1.00 | 1.42 | 1.77 | NM_001975 | Hs.511915 | ENO2 |
| 308 | 208637_x_at | 1397.08 | 0.99 | 1.37 | 1.68 | BC003576 | Hs.119000 | ACTN1 |
| 309 | 221973_at | 1400.60 | 1.00 | 1.40 | 1.74 | AI983904 | Hs.446532 | LOC150759 |
| 310 | 209257_s_at | 1404.17 | 1.01 | 1.26 | 1.46 | BF795297 | Hs.24485 | CSPG6 |
| 311 | 221664_s_at | 1405.22 | 1.02 | 1.37 | 1.68 | AF154005 | Hs.506845 | F11R |
| 312 | 203105_s_at | 1414.16 | 1.06 | 1.43 | 1.81 | NM_012062 | Hs.180628 | DNM1L |
| 313 | 212201_at | 1416.64 | 1.06 | 1.40 | 1.74 | AW274877 | Hs.524874 | KIAA0692 |
| 314 | 205249_at | 1417.87 | 1.06 | 1.36 | 1.65 | NM_000399 | Hs.1395 | EGR2 |
| 315 | 218344_s_at | 1419.49 | 1.07 | 1.42 | 1.78 | NM_018254 | Hs.125478 | RCOR3 |
| 316 | 203936_s_at | 1419.63 | 1.07 | 1.35 | 1.64 | NM_004994 | Hs.151738 | MMP9 |
| 317 | 221427_s_at | 1422.02 | 1.07 | 1.43 | 1.79 | NM_030937 | Hs.143601 | CCNL2 |
| 318 | 217763_s_at | 1422.34 | 1.07 | 1.33 | 1.61 | NM_006868 | Hs.223025 | RAB31 |
| 319 | 215201_at | 1428.09 | 1.09 | 1.41 | 1.75 | AW166925 | Hs.333141 | REPS1 |
| 320 | 212998_x_at | 1429.23 | 1.09 | 1.41 | 1.76 | AI583173 | Hs.409934 | HLA-DQB1 |
| 321 | 216899_s_at | 1431.04 | 1.10 | 1.40 | 1.73 | AC003999 | Hs.410745 | SCAP2 |
| 322 | 219094_at | 1433.34 | 1.11 | 1.40 | 1.74 | NM_014154 | Hs.102708 | ARMC8 |
| 323 | 220202_s_at | 1433.38 | 1.11 | 1.41 | 1.75 | NM_018835 | Hs.112227 | MNAB |
| 324 | 215176_x_at | 1434.17 | 1.11 | 1.33 | 1.59 | AW404894 | Hs.526208 | — |
| 325 | 212271_at | 1436.79 | 1.13 | 1.41 | 1.75 | AA195999 | Hs.324473 | MAPK1 |
| 326 | 219577_s_at | 1438.27 | 1.13 | 1.37 | 1.67 | NM_019112 | Hs.134514 | ABCA7 |
| 327 | 215047_at | 1445.59 | 1.16 | 1.41 | 1.77 | AL080170 | — | BIA2 |
| 328 | 207067_s_at | 1446.86 | 1.16 | 1.40 | 1.74 | NM_002112 | Hs.1481 | HDC |
| 329 | 206049_at | 1453.32 | 1.19 | 1.38 | 1.70 | NM_003005 | Hs.73800 | SELP |
| 330 | 217823_s_at | 1456.37 | 1.19 | 1.40 | 1.74 | AL562526 | Hs.184325 | UBE2J1 |
| 331 | 201089_at | 1461.33 | 1.22 | 1.41 | 1.75 | NM_001693 | Hs.295917 | ATP6V1B2 |
| 332 | 201151_s_at | 1461.79 | 1.21 | 1.37 | 1.68 | BF512200 | Hs.28578 | MBNL1 |
| 333 | 37966_at | 1464.21 | 1.21 | 1.37 | 1.68 | AA187563 | Hs.8836 | PARVB |
| 334 | 207791_s_at | 1464.57 | 1.21 | 1.39 | 1.72 | NM_004161 | Hs.227327 | RAB1A |
| 335 | 211546_x_at | 1464.79 | 1.21 | 1.39 | 1.71 | L36674 | Hs.76930 | SNCA |
| 336 | 201689_s_at | 1468.24 | 1.21 | 1.41 | 1.76 | BE974098 | Hs.162089 | TPD52 |
| 337 | 203337_s_at | 1468.52 | 1.21 | 1.36 | 1.66 | NM_004763 | Hs.200666 | ITGB1BP1 |
| 338 | 221752_at | 1468.67 | 1.20 | 1.41 | 1.76 | AL041728 | Hs.60377 | SSH1 |
| 339 | 208702_x_at | 1471.44 | 1.21 | 1.40 | 1.74 | AI525212 | Hs.279518 | APLP2 |
| 340 | 202164_s_at | 1472.58 | 1.22 | 1.43 | 1.80 | AF180476 | Hs.26703 | CNOT8 |
| 341 | 218751_s_at | 1473.54 | 1.21 | 1.38 | 1.70 | NM_018315 | Hs.312503 | FBXW7 |
| 342 | 205612_at | 1475.25 | 1.22 | 1.38 | 1.70 | NM_007351 | Hs.268107 | MMRN1 |
| 343 | 216834_at | 1477.07 | 1.22 | 1.39 | 1.72 | S59049 | Hs.75256 | RGS1 |
| 344 | 212062_at | 1479.70 | 1.23 | 1.36 | 1.65 | AB014511 | Hs.406434 | ATP9A |
| 345 | 201279_s_at | 1480.21 | 1.23 | 1.34 | 1.62 | BC003064 | Hs.81988 | DAB2 |
| 346 | 221449_s_at | 1486.52 | 1.25 | 1.40 | 1.73 | NM_030790 | Hs.23047 | CDA08 |
| 347 | 209710_at | 1487.17 | 1.25 | 1.41 | 1.75 | AL563460 | Hs.367725 | GATA2 |
| 348 | 35626_at | 1490.03 | 1.25 | 1.37 | 1.68 | U30894 | Hs.31074 | SGSH |
| 349 | 201136_at | 1490.45 | 1.25 | 1.46 | 1.85 | NM_002668 | Hs.77422 | PLP2 |
| 350 | 203085_s_at | 1491.00 | 1.25 | 1.43 | 1.79 | BC000125 | — | — |
| 351 | 204042_at | 1493.48 | 1.26 | 1.32 | 1.57 | AB020707 | Hs.82318 | WASF3 |
| 352 | 215147_at | 1493.52 | 1.26 | 1.40 | 1.74 | AF007147 | Hs.113166 | — |
| 353 | 208997_s_at | 1494.38 | 1.25 | 1.36 | 1.66 | U82819 | Hs.80658 | UCP2 |
| 354 | 203520_s_at | 1495.10 | 1.25 | 1.40 | 1.74 | AW613549 | Hs.147868 | ZNF318 |
| 355 | 213546_at | 1495.66 | 1.26 | 1.39 | 1.72 | AL050378 | Hs.112423 | DKFZp586I1420 |
| 356 | 202859_x_at | 1496.43 | 1.26 | 1.04 | 1.07 | NM_000584 | Hs.624 | IL8 |
| 357 | 212605_s_at | 1496.57 | 1.26 | 1.38 | 1.69 | AK025759 | Hs.188882 | — |
| 358 | 213872_at | 1498.55 | 1.26 | 1.33 | 1.60 | BE465032 | — | — |
| 359 | 221669_s_at | 1500.87 | 1.26 | 1.42 | 1.79 | BC001964 | Hs.14791 | ACAD8 |
| 360 | 201858_s_at | 1503.47 | 1.27 | 1.39 | 1.72 | J03223 | Hs.1908 | PRG1 |
| 361 | 213418_at | 1506.09 | 1.27 | 1.36 | 1.66 | NM_002155 | Hs.3268 | HSPA6 |
| 362 | 213518_at | 1514.98 | 1.31 | 1.42 | 1.78 | AI689429 | Hs.496511 | PRKCI |
| 363 | 204060_s_at | 1515.87 | 1.31 | 1.40 | 1.73 | NM_005044 | Hs.147996 | PRKX /// PRKY |
| 364 | 221253_s_at | 1516.74 | 1.32 | 1.45 | 1.84 | NM_030810 | Hs.430169 | TXNDC5 |
| 365 | 203414_at | 1518.85 | 1.33 | 1.38 | 1.69 | NM_012329 | Hs.79889 | MMD |
| 366 | 206245_s_at | 1525.47 | 1.36 | 1.41 | 1.75 | NM_006469 | Hs.197298 | IVNS1ABP |
| 367 | 203817_at | 1525.74 | 1.36 | 1.36 | 1.66 | W93728 | Hs.77890 | GUCY1B3 |
| 368 | 218733_at | 1527.64 | 1.36 | 1.38 | 1.69 | NM_018133 | Hs.518278 | FLJ10546 |
| 369 | 202593_s_at | 1528.23 | 1.37 | 1.40 | 1.73 | NM_016641 | Hs.512607 | MIR16 |
| 370 | 210042_s_at | 1538.39 | 1.43 | 1.36 | 1.65 | AF073890 | — | — |
| 371 | 215121_x_at | 1542.93 | 1.43 | 1.37 | 1.67 | AA680302 | Hs.405944 | IGLC2 |
| 372 | 212038_s_at | 1543.57 | 1.43 | 1.39 | 1.72 | AL515918 | Hs.404814 | VDAC1 |
| 373 | 201341_at | 1546.40 | 1.45 | 1.40 | 1.74 | NM_003633 | Hs.104925 | ENC1 |
| 374 | 207426_s_at | 1549.72 | 1.45 | 1.25 | 1.45 | NM_003326 | Hs.181097 | TNFSF4 |
| 375 | 208811_s_at | 1552.31 | 1.46 | 1.38 | 1.70 | AF080569 | Hs.181195 | DNAJB6 |
| 376 | 206278_at | 1553.21 | 1.45 | 1.38 | 1.69 | D10202 | Hs.46 | PTAFR |
| 377 | 201028_s_at | 1553.79 | 1.45 | 1.38 | 1.70 | U82164 | Hs.283477 | CD99 |
| 378 | 206522_at | 1554.13 | 1.46 | 1.34 | 1.61 | NM_004668 | Hs.122785 | MGAM |
| 379 | 206881_s_at | 1557.54 | 1.49 | 1.36 | 1.65 | NM_006865 | Hs.113277 | LILRA3 |
| 380 | 202974_at | 1557.55 | 1.49 | 1.34 | 1.61 | NM_002436 | Hs.422215 | MPP1 |
| 381 | 209616_s_at | 1558.82 | 1.49 | 1.34 | 1.62 | S73751 | Hs.278997 | CES1 |
| 382 | 200931_s_at | 1558.95 | 1.49 | 1.39 | 1.71 | NM_014000 | Hs.75350 | VCL |
| 383 | 203820_s_at | 1559.26 | 1.48 | 1.36 | 1.66 | NM_006547 | Hs.79440 | IMP-3 |
| 384 | 218364_at | 1563.63 | 1.51 | 1.37 | 1.68 | NM_017724 | Hs.268768 | LRRFIP2 |
| 385 | 220354_at | 1566.01 | 1.51 | 1.37 | 1.68 | NM_016616 | Hs.134079 | TXNDC3 |
| 386 | 207543_s_at | 1567.61 | 1.51 | 1.40 | 1.74 | NM_000917 | Hs.76768 | P4HA1 |
| 387 | 203280_at | 1569.39 | 1.51 | 1.38 | 1.71 | NM_014649 | Hs.159384 | SAFB2 |
| 388 | 222030_at | 1571.28 | 1.51 | 1.34 | 1.62 | AW024335 | Hs.112058 | SIVA |
| 389 | 203923_s_at | 1572.02 | 1.51 | 1.34 | 1.62 | NM_000397 | Hs.88974 | CYBB |
| 390 | 207124_s_at | 1572.47 | 1.51 | 1.38 | 1.69 | NM_006578 | Hs.155090 | GNB5 |
| 391 | 203146_s_at | 1574.37 | 1.52 | 1.38 | 1.69 | NM_001470 | Hs.167017 | GABBR1 |
| 392 | 219634_at | 1577.16 | 1.55 | 1.36 | 1.66 | NM_018413 | Hs.433059 | CHST11 |
| 393 | 214359_s_at | 1579.24 | 1.56 | 1.38 | 1.69 | AI218219 | Hs.74335 | HSPCB |
| 394 | 210401_at | 1580.18 | 1.56 | 1.39 | 1.72 | U45448 | Hs.41735 | P2RX1 |
| 395 | 208763_s_at | 1582.39 | 1.57 | 1.38 | 1.69 | AL110191 | Hs.420569 | DSIPI |
| 396 | 200593_s_at | 1586.51 | 1.60 | 1.38 | 1.70 | BC003621 | Hs.166463 | HNRPU |
| 397 | 212016_s_at | 1589.05 | 1.60 | 1.38 | 1.69 | AA679988 | Hs.172550 | PTBP1 |
| 398 | 214895_s_at | 1590.18 | 1.60 | 1.40 | 1.73 | AU135154 | Hs.172028 | ADAM10 |
| 399 | 211749_s_at | 1590.85 | 1.60 | 1.36 | 1.66 | BC005941 | Hs.66708 | VAMP3 |
| 400 | 221430_s_at | 1593.05 | 1.61 | 1.41 | 1.75 | NM_030963 | Hs.267120 | RNF146 |
| 401 | 203315_at | 1595.26 | 1.62 | 1.36 | 1.65 | BC000103 | Hs.101695 | NCK2 |
| 402 | 219657_s_at | 1595.79 | 1.62 | 1.37 | 1.68 | NM_016531 | Hs.145754 | KLF3 |
| 403 | 206544_x_at | 1598.43 | 1.64 | 1.39 | 1.71 | NM_003070 | Hs.396404 | SMARCA2 |
| 404 | 219957_at | 1600.14 | 1.64 | 1.41 | 1.75 | NM_017987 | — | — |
| 405 | 210681_s_at | 1601.35 | 1.65 | 1.36 | 1.66 | AF153604 | Hs.339425 | USP15 |
| 406 | 218175_at | 1601.78 | 1.65 | 1.36 | 1.66 | NM_025140 | Hs.387266 | FLJ22471 |
| 407 | 200661_at | 1605.92 | 1.66 | 1.35 | 1.64 | NM_000308 | Hs.118126 | PPGB |
| 408 | 215285_s_at | 1606.71 | 1.66 | 1.39 | 1.72 | AA927671 | Hs.123637 | PHTF1 |
| 409 | 208453_s_at | 1606.88 | 1.66 | 1.38 | 1.70 | NM_006523 | Hs.390623 | XPNPEP1 |
| 410 | 200962_at | 1608.32 | 1.66 | 1.35 | 1.65 | AI348010 | Hs.375921 | RPL31 |
| 411 | 213653_at | 1614.83 | 1.71 | 1.40 | 1.74 | AW069290 | Hs.168799 | METTL3 |
| 412 | 209861_s_at | 1620.95 | 1.74 | 1.39 | 1.72 | U13261 | Hs.144906 | METAP2 |
| 413 | 214291_at | 1621.95 | 1.74 | 1.39 | 1.71 | AA522618 | Hs.374588 | RPL17 |
| 414 | 204908_s_at | 1622.77 | 1.74 | 1.36 | 1.65 | NM_005178 | Hs.31210 | BCL3 |
| 415 | 206478_at | 1623.38 | 1.74 | 1.40 | 1.73 | NM_014792 | Hs.38365 | KIAA0125 |
| 416 | 36711_at | 1624.74 | 1.75 | 1.09 | 1.16 | AL021977 | Hs.460889 | MAFF |
| 417 | 207078_at | 1625.20 | 1.75 | 1.37 | 1.69 | NM_005466 | Hs.167738 | MED6 |
| 418 | 209906_at | 1625.50 | 1.74 | 1.30 | 1.53 | U62027 | Hs.155935 | C3AR1 |
| 419 | 217764_s_at | 1626.92 | 1.75 | 1.37 | 1.69 | AF183421 | Hs.223025 | RAB31 |
| 420 | 201109_s_at | 1627.74 | 1.75 | 1.37 | 1.67 | AV726673 | Hs.164226 | THBS1 |
| 421 | 214290_s_at | 1631.55 | 1.77 | 1.19 | 1.34 | AI313324 | Hs.417332 | HIST2H2AA |
| 422 | 200790_at | 1631.66 | 1.77 | 1.36 | 1.65 | NM_002539 | Hs.443409 | ODC1 |
| 423 | 217783_s_at | 1632.39 | 1.78 | 1.30 | 1.53 | NM_016061 | Hs.184542 | YPEL5 |
| 424 | 205513_at | 1637.73 | 1.79 | 1.58 | 2.13 | NM_001062 | Hs.2012 | TCN1 |
| 425 | 216727_at | 1639.16 | 1.80 | 1.35 | 1.63 | AF034187 | Hs.367811 | STK38 |
| 426 | 204482_at | 1643.20 | 1.83 | 1.33 | 1.59 | NM_003277 | Hs.110903 | CLDN5 |
| 427 | 211555_s_at | 1643.62 | 1.83 | 1.35 | 1.65 | AF020340 | Hs.77890 | GUCY1B3 |
| 428 | 204198_s_at | 1648.82 | 1.86 | 1.36 | 1.67 | AA541630 | Hs.170019 | RUNX3 |
| 429 | 203482_at | 1651.37 | 1.87 | 1.37 | 1.68 | AL133215 | Hs.447458 | C10orf6 |
| 430 | 207431_s_at | 1651.57 | 1.87 | 1.36 | 1.65 | NM_003676 | Hs.299878 | DEGS |
| 431 | 202738_s_at | 1653.84 | 1.88 | 1.38 | 1.71 | BG149218 | Hs.78060 | PHKB |
| 432 | 200999_s_at | 1653.95 | 1.88 | 1.35 | 1.63 | NM_006825 | Hs.74368 | CKAP4 |
| 433 | 202644_s_at | 1657.53 | 1.90 | 1.34 | 1.61 | NM_006290 | Hs.211600 | TNFAIP3 |
| 434 | 204369_at | 1660.07 | 1.91 | 1.39 | 1.71 | NM_006218 | Hs.85701 | PIK3CA |
| 435 | 212762_s_at | 1660.12 | 1.90 | 1.26 | 1.46 | AI375916 | Hs.214039 | TCF7L2 |
| 436 | 217427_s_at | 1664.34 | 1.93 | 1.25 | 1.45 | X75296 | Hs.415735 | HIRA |
| 437 | 222018_at | 1665.62 | 1.93 | 1.34 | 1.62 | AI992187 | Hs.32916 | NACA |
| 438 | 212361_s_at | 1668.53 | 1.95 | 1.39 | 1.72 | AW190070 | Hs.374535 | ATP2A2 |
| 439 | 211744_s_at | 1674.10 | 1.96 | 1.29 | 1.52 | BC005930 | Hs.75626 | CD58 |
| 440 | 219551_at | 1676.18 | 1.97 | 1.35 | 1.63 | NM_018456 | Hs.383018 | EAF2 |
| 441 | 210621_s_at | 1683.85 | 2.02 | 1.39 | 1.72 | M23612 | Hs.758 | RASA1 |
| 442 | 206632_s_at | 1686.23 | 2.03 | 1.42 | 1.78 | NM_004900 | Hs.226307 | APOBEC3B |
| 443 | 211776_s_at | 1688.55 | 2.03 | 1.37 | 1.67 | BC006141 | Hs.103839 | EPB41L3 |
| 444 | 221919_at | 1692.61 | 2.06 | 1.37 | 1.68 | AW450929 | Hs.356721 | HNRPA1 |
| 445 | 214073_at | 1692.93 | 2.06 | 1.21 | 1.38 | BG475299 | Hs.301348 | CTTN |
| 446 | 211645_x_at | 1694.26 | 2.06 | 1.36 | 1.65 | M85256 | Hs.512133 | — |
| 447 | 218280_x_at | 1694.74 | 2.06 | 1.20 | 1.35 | NM_003516 | Hs.417332 | HIST2H2AA |
| 448 | 203725_at | 1696.57 | 2.08 | 1.24 | 1.41 | NM_001924 | Hs.80409 | GADD45A |
| 449 | 220477_s_at | 1697.25 | 2.08 | 1.34 | 1.62 | NM_014145 | Hs.380990 | C20orf30 |
| 450 | 202284_s_at | 1698.97 | 2.10 | 1.34 | 1.61 | NM_000389 | Hs.370771 | CDKN1A |
| 451 | 206666_at | 1701.03 | 2.11 | 1.17 | 1.29 | NM_002104 | Hs.277937 | GZMK |
| 452 | 213746_s_at | 1704.00 | 2.12 | 1.35 | 1.65 | AW051856 | Hs.195464 | FLNA |
| 453 | 210191_s_at | 1704.83 | 2.12 | 1.38 | 1.69 | BC002447 | Hs.123637 | PHTF1 |
| 454 | 212428_at | 1705.70 | 2.13 | 1.37 | 1.68 | AW001101 | Hs.445255 | KIAA0368 |
| 455 | 203375_s_at | 1706.68 | 2.14 | 1.29 | 1.53 | NM_003291 | — | — |
| 456 | 204467_s_at | 1714.00 | 2.20 | 1.31 | 1.57 | NM_000345 | Hs.76930 | SNCA |
| 457 | 206624_at | 1714.16 | 2.19 | 1.38 | 1.69 | NM_004654 | Hs.371255 | USP9Y |
| 458 | 204500_s_at | 1720.70 | 2.24 | 1.36 | 1.65 | NM_015239 | Hs.21542 | AGTPBP1 |
| 459 | 210830_s_at | 1721.29 | 2.23 | 1.37 | 1.68 | AF001602 | Hs.165598 | PON2 |
| 460 | 208753_s_at | 1723.57 | 2.26 | 1.36 | 1.65 | BC002387 | Hs.419776 | NAP1L1 |
| 461 | 201121_s_at | 1725.62 | 2.26 | 1.31 | 1.57 | NM_006667 | Hs.90061 | PGRMC1 |
| 462 | 216942_s_at | 1726.42 | 2.26 | 1.31 | 1.55 | D28586 | Hs.75626 | CD58 |
| 463 | 210742_at | 1732.90 | 2.30 | 1.37 | 1.68 | AF064103 | Hs.405214 | CDC14A |
| 464 | 207815_at | 1735.40 | 2.33 | 1.24 | 1.42 | NM_002620 | Hs.72933 | PF4V1 |
| 465 | 214060_at | 1736.21 | 2.33 | 1.38 | 1.69 | BE220360 | Hs.923 | SSBP1 |
| 466 | 201104_x_at | 1736.98 | 2.33 | 1.37 | 1.68 | NM_015383 | Hs.445556 | LOC376745 /// |
| | | | | | | | | DJ328E19.C1.1 /// |
| | | | | | | | | LOC200030 /// |
| | | | | | | | | MGC8902 |
| 467 | 221269_s_at | 1738.33 | 2.34 | 1.35 | 1.63 | NM_031286 | Hs.109051 | SH3BGRL3 |
| 468 | 201545_s_at | 1747.15 | 2.43 | 1.36 | 1.66 | NM_004643 | Hs.117176 | PABPN1 |
| 469 | 214958_s_at | 1749.07 | 2.44 | 1.35 | 1.64 | AK021738 | Hs.16165 | EVER1 |
| 470 | 215322_at | 1749.95 | 2.44 | 1.31 | 1.55 | AL080190 | Hs.189242 | — |
| 471 | 212542_s_at | 1753.53 | 2.46 | 1.35 | 1.64 | BF224151 | Hs.9927 | PHIP |
| 472 | 218243_at | 1756.86 | 2.49 | 1.24 | 1.43 | NM_025158 | Hs.306769 | RUFY1 |
| 473 | 207000_s_at | 1759.16 | 2.50 | 1.37 | 1.67 | NM_005605 | Hs.75206 | PPP3CC |
| 474 | 200604_s_at | 1760.34 | 2.51 | 1.36 | 1.65 | M18468 | Hs.280342 | PRKAR1A |
| 475 | 201560_at | 1761.16 | 2.51 | 1.34 | 1.62 | NM_013943 | Hs.25035 | CLIC4 |
| 476 | 202928_s_at | 1764.94 | 2.53 | 1.35 | 1.64 | NM_024165 | Hs.166204 | PHF1 |
| 477 | 207957_s_at | 1766.34 | 2.53 | 1.38 | 1.69 | NM_002738 | Hs.349845 | PRKCB1 |
| 478 | 212999_x_at | 1766.65 | 2.52 | 1.11 | 1.19 | AW276186 | Hs.409934 | HLA-DQB1 |
| 479 | 201574_at | 1767.65 | 2.53 | 1.37 | 1.67 | NM_004730 | Hs.77324 | ETF1 |
| 480 | 221478_at | 1769.34 | 2.53 | 1.35 | 1.64 | AL132665 | Hs.132955 | BNIP3L |
| 481 | 207206_s_at | 1770.83 | 2.53 | 1.31 | 1.57 | NM_000697 | Hs.1200 | ALOX12 |
| 482 | 201794_s_at | 1771.21 | 2.52 | 1.28 | 1.49 | NM_014837 | Hs.43660 | C1orf16 |
| 483 | 210423_s_at | 1772.78 | 2.53 | 1.27 | 1.48 | L32185 | Hs.135163 | SLC11A1 |
| 484 | 208730_x_at | 1773.91 | 2.52 | 1.36 | 1.65 | AA535244 | Hs.78305 | RAB2 |
| 485 | 212667_at | 1775.31 | 2.53 | 1.33 | 1.61 | AL575922 | Hs.111779 | SPARC |
| 486 | 206964_at | 1779.91 | 2.57 | 1.21 | 1.37 | NM_016347 | Hs.14637 | NAT8 |
| 487 | 209095_at | 1780.81 | 2.57 | 1.38 | 1.70 | J03620 | Hs.74635 | DLD |
| 488 | 213758_at | 1784.88 | 2.59 | 1.35 | 1.63 | AW337510 | Hs.433419 | COX4I1 |
| 489 | 219983_at | 1786.69 | 2.61 | 1.24 | 1.43 | NM_020386 | Hs.36761 | HRASLS |
| 490 | 220046_s_at | 1789.31 | 2.63 | 1.32 | 1.58 | NM_020307 | Hs.4859 | CCNL1 |
| 491 | 214414_x_at | 1791.20 | 2.65 | 1.25 | 1.45 | T50399 | Hs.398636 | HBA2 |
| 492 | 201229_s_at | 1795.51 | 2.67 | 1.37 | 1.68 | BC000422 | Hs.241558 | ARIH2 |
| 493 | 201399_s_at | 1798.80 | 2.69 | 1.31 | 1.55 | NM_014294 | Hs.4147 | TRAM1 |
| 494 | 212843_at | 1799.33 | 2.68 | 1.15 | 1.26 | AA126505 | Hs.78792 | NCAM1 |
| 495 | 207474_at | 1800.42 | 2.69 | 1.29 | 1.53 | NM_017719 | Hs.79025 | SNRK |
| 496 | 219607_s_at | 1805.46 | 2.74 | 1.32 | 1.57 | NM_024021 | Hs.325960 | MS4A4A |
| 497 | 218064_s_at | 1808.81 | 2.75 | 1.36 | 1.66 | NM_014371 | Hs.96200 | AKAP8L |
| 498 | 209605_at | 1809.08 | 2.75 | 1.28 | 1.49 | D87292 | Hs.351863 | TST |
| 499 | 210299_s_at | 1810.43 | 2.77 | 1.32 | 1.58 | AF063002 | Hs.421383 | FHL1 |
| 500 | 208786_s_at | 1811.61 | 2.77 | 1.34 | 1.61 | AF183417 | Hs.356061 | MAP1LC3B |
| 501 | 212878_s_at | 1813.06 | 2.77 | 1.34 | 1.62 | AA284075 | Hs.20107 | KNS2 |
| 502 | 213385_at | 1814.95 | 2.77 | 1.33 | 1.59 | AK026415 | Hs.407520 | CHN2 |
| 503 | 218559_s_at | 1815.84 | 2.77 | 1.33 | 1.60 | NM_005461 | Hs.169487 | MAFB |
| 504 | 212614_at | 1817.78 | 2.78 | 1.36 | 1.65 | BG285011 | Hs.12702 | ARID5B |
| 505 | 205127_at | 1820.12 | 2.79 | 1.34 | 1.62 | NM_000962 | Hs.88474 | PTGS1 |
| 506 | 215038_s_at | 1820.44 | 2.79 | 1.36 | 1.66 | AF049103 | Hs.6947 | HYPB |
| 507 | 219681_s_at | 1821.52 | 2.79 | 1.35 | 1.63 | NM_025151 | Hs.96125 | RAB11FIP1 |
| 508 | 220712_at | 1821.69 | 2.79 | 1.33 | 1.59 | NM_024984 | — | — |
| 509 | 202696_at | 1822.30 | 2.79 | 1.36 | 1.65 | NM_005109 | Hs.95220 | OSR1 |
| 510 | 209306_s_at | 1823.14 | 2.78 | 1.37 | 1.67 | AI139569 | Hs.153026 | SWAP70 |
| 511 | 206871_at | 1826.96 | 2.81 | 1.37 | 1.68 | NM_001972 | Hs.99863 | ELA2 |
| 512 | 200927_s_at | 1828.26 | 2.82 | 1.36 | 1.66 | AA919115 | Hs.5807 | RAB14 |
| 513 | 215483_at | 1829.90 | 2.83 | 1.23 | 1.40 | AK000270 | Hs.58103 | AKAP9 |
| 514 | 203940_s_at | 1831.28 | 2.83 | 1.31 | 1.56 | NM_014909 | Hs.155182 | KIAA1036 |
| 515 | 218935_at | 1837.41 | 2.87 | 1.28 | 1.50 | NM_014600 | Hs.368808 | EHD3 |
| 516 | 214455_at | 1848.57 | 2.96 | 1.11 | 1.19 | NM_003526 | — | — |
| 517 | 200622_x_at | 1859.16 | 3.06 | 1.26 | 1.45 | AV685208 | Hs.334330 | CALM3 |
| 518 | 206130_s_at | 1860.39 | 3.07 | 1.35 | 1.64 | NM_001181 | Hs.1259 | ASGR2 |
| 519 | 204285_s_at | 1861.09 | 3.07 | 1.09 | 1.15 | AI857639 | Hs.96 | PMAIP1 |
| 520 | 201743_at | 1862.39 | 3.07 | 1.33 | 1.60 | NM_000591 | Hs.163867 | CD14 |
| 521 | 215224_at | 1863.51 | 3.08 | 1.35 | 1.63 | AK025200 | Hs.406300 | RPL23 |
| 522 | 219382_at | 1864.40 | 3.09 | 1.34 | 1.63 | NM_013368 | Hs.169138 | SERTAD3 |
| 523 | 203419_at | 1865.39 | 3.09 | 1.34 | 1.63 | NM_014727 | Hs.92236 | MLL4 |
| 524 | 214513_s_at | 1865.66 | 3.08 | 1.34 | 1.61 | M34356 | Hs.22315 | CREB1 |
| 525 | 201251_at | 1868.55 | 3.10 | 1.35 | 1.63 | NM_002654 | Hs.198281 | PKM2 |
| 526 | 202978_s_at | 1868.59 | 3.09 | 1.36 | 1.66 | AW204564 | Hs.321022 | ZF |
| 527 | 202856_s_at | 1873.32 | 3.13 | 0.92 | 0.88 | NM_004207 | Hs.386678 | SLC16A3 |
| 528 | 218454_at | 1873.55 | 3.13 | 1.35 | 1.63 | NM_024829 | Hs.178470 | FLJ22662 |
| 529 | 203603_s_at | 1878.71 | 3.17 | 1.33 | 1.59 | NM_014795 | Hs.34871 | ZFHX1B |
| 530 | 207156_at | 1879.35 | 3.17 | 1.21 | 1.37 | NM_021064 | Hs.51011 | HIST1H2AG |
| 531 | 221718_s_at | 1881.90 | 3.19 | 1.33 | 1.60 | M90360 | Hs.350631 | AKAP13 |
| 532 | 203591_s_at | 1882.40 | 3.19 | 1.32 | 1.58 | NM_000760 | Hs.381027 | CSF3R |
| 533 | 211654_x_at | 1883.83 | 3.20 | 1.22 | 1.39 | M17565 | Hs.409934 | HLA-DQB1 |
| 534 | 217028_at | 1885.11 | 3.21 | 1.35 | 1.64 | AJ224869 | Hs.421986 | CXCR4 |
| 535 | 210337_s_at | 1886.00 | 3.21 | 1.34 | 1.62 | U18197 | Hs.387567 | ACLY |
| 536 | 201337_s_at | 1886.06 | 3.20 | 1.30 | 1.54 | NM_004781 | Hs.66708 | VAMP3 |
| 537 | 219694_at | 1887.37 | 3.21 | 1.34 | 1.61 | NM_019018 | Hs.528644 | FLJ11127 |
| 538 | 209882_at | 1891.50 | 3.24 | 1.24 | 1.42 | AF084462 | Hs.446472 | RIT1 |
| 539 | 209053_s_at | 1892.39 | 3.24 | 1.35 | 1.63 | BE793789 | Hs.110457 | WHSC1 |
| 540 | 211936_at | 1901.77 | 3.31 | 1.30 | 1.54 | AF216292 | Hs.310769 | HSPA5 |
| 541 | 209773_s_at | 1903.40 | 3.32 | 1.30 | 1.54 | BC001886 | Hs.226390 | RRM2 |
| 542 | 202762_at | 1903.68 | 3.32 | 1.34 | 1.62 | AL049383 | Hs.58617 | ROCK2 |
| 543 | 209067_s_at | 1904.19 | 3.32 | 1.35 | 1.64 | D89092 | Hs.372673 | HNRPDL |
| 544 | 209610_s_at | 1904.45 | 3.32 | 1.34 | 1.61 | BF340083 | Hs.323878 | SLC1A4 |
| 545 | 209641_s_at | 1906.52 | 3.34 | 1.31 | 1.56 | AF009670 | Hs.90786 | ABCC3 |
| 546 | 202381_at | 1907.10 | 3.34 | 1.35 | 1.64 | NM_003816 | Hs.2442 | ADAM9 |
| 547 | 201502_s_at | 1907.32 | 3.34 | 1.15 | 1.26 | AI078167 | Hs.81328 | NFKBIA |
| 548 | 203140_at | 1908.59 | 3.34 | 1.32 | 1.58 | NM_001706 | Hs.155024 | BCL6 |
| 549 | 206542_s_at | 1911.47 | 3.37 | 1.35 | 1.64 | AV725365 | Hs.396404 | SMARCA2 |
| 550 | 213594_x_at | 1912.43 | 3.37 | 1.34 | 1.62 | AU130523 | Hs.3530 | FUSIP1 |
| 551 | 210357_s_at | 1915.72 | 3.38 | 1.26 | 1.46 | BC000669 | Hs.433337 | SMOX |
| 552 | 208853_s_at | 1918.58 | 3.40 | 1.34 | 1.61 | L18887 | Hs.155560 | CANX |
| 553 | 216278_at | 1919.34 | 3.40 | 1.35 | 1.64 | AL109705 | Hs.9997 | — |
| 554 | 203017_s_at | 1919.85 | 3.40 | 1.30 | 1.54 | R52678 | Hs.22587 | SSX2IP |
| 555 | 203198_at | 1920.26 | 3.39 | 1.35 | 1.64 | NM_001261 | Hs.150423 | CDK9 |
| 556 | 213502_x_at | 1920.49 | 3.39 | 1.36 | 1.65 | AA398569 | Hs.272302 | LOC91316 |
| 557 | 209396_s_at | 1924.30 | 3.42 | 1.36 | 1.65 | M80927 | Hs.382202 | CHI3L1 |
| 558 | 209892_at | 1926.40 | 3.42 | 1.34 | 1.61 | AF305083 | Hs.390420 | FUT4 |
| 559 | 215716_s_at | 1926.73 | 3.42 | 1.32 | 1.58 | L14561 | Hs.20952 | ATP2B1 |
| 560 | 209344_at | 1927.02 | 3.42 | 1.28 | 1.50 | BC002827 | Hs.250641 | TPM4 |
| 561 | 201663_s_at | 1929.52 | 3.43 | 1.35 | 1.64 | NM_005496 | Hs.50758 | SMC4L1 |
| 562 | 221210_s_at | 1930.38 | 3.43 | 1.32 | 1.58 | NM_030769 | Hs.64896 | NPL |
| 563 | 209984_at | 1931.85 | 3.43 | 1.35 | 1.64 | AB037901 | Hs.209292 | JMJD2C |
| 564 | 202728_s_at | 1934.44 | 3.46 | 1.24 | 1.42 | AI986120 | Hs.241257 | LTBP1 |
| 565 | 200866_s_at | 1934.69 | 3.45 | 1.32 | 1.58 | M32221 | Hs.406455 | PSAP |
| 566 | 209498_at | 1936.42 | 3.46 | 1.39 | 1.71 | X16354 | — | — |
| 567 | 218943_s_at | 1936.94 | 3.46 | 1.28 | 1.49 | NM_014314 | Hs.438386 | DDX58 |
| 568 | 202729_s_at | 1937.41 | 3.46 | 1.27 | 1.48 | NM_000627 | Hs.241257 | LTBP1 |
| 569 | 205159_at | 1940.50 | 3.49 | 1.33 | 1.59 | AV756141 | Hs.285401 | CSF2RB |
| 570 | 209586_s_at | 1945.09 | 3.51 | 1.29 | 1.52 | AF123539 | — | HTCD37 |
| 571 | 211366_x_at | 1945.24 | 3.51 | 1.33 | 1.59 | U13698 | Hs.2490 | CASP1 |
| 572 | 211926_s_at | 1945.97 | 3.51 | 1.34 | 1.62 | AI827941 | Hs.146550 | MYH9 |
| 573 | 210240_s_at | 1946.67 | 3.52 | 1.33 | 1.61 | U20498 | Hs.435051 | CDKN2D |
| 574 | 202450_s_at | 1948.39 | 3.54 | 1.29 | 1.53 | NM_000396 | Hs.83942 | CTSK |
| 575 | 201639_s_at | 1948.98 | 3.53 | 1.30 | 1.53 | NM_013291 | Hs.83727 | CPSF1 |
| 576 | 215111_s_at | 1949.37 | 3.53 | 1.21 | 1.38 | AK027071 | Hs.114360 | TGFB1I4 |
| 577 | 200709_at | 1951.64 | 3.55 | 1.32 | 1.58 | NM_000801 | Hs.374638 | FKBP1A |
| 578 | 202364_at | 1952.51 | 3.54 | 1.33 | 1.60 | NM_005962 | Hs.118630 | MXI1 |
| 579 | 220494_s_at | 1953.40 | 3.55 | 1.21 | 1.36 | NM_018678 | — | — |
| 580 | 207667_s_at | 1954.30 | 3.54 | 1.32 | 1.57 | NM_002756 | Hs.180533 | MAP2K3 |
| 581 | 216765_at | 1954.67 | 3.54 | 1.31 | 1.56 | AK025177 | Hs.436145 | MAP2K5 |
| 582 | 201423_s_at | 1955.11 | 3.54 | 1.34 | 1.62 | AL037208 | Hs.270788 | CUL4A |
| 583 | 208498_s_at | 1955.79 | 3.54 | 1.29 | 1.52 | NM_004038 | Hs.288166 | AMY2B |
| 584 | 202113_s_at | 1955.92 | 3.54 | 1.33 | 1.60 | AF043453 | Hs.11183 | SNX2 |
| 585 | 57588_at | 1956.00 | 3.54 | 1.30 | 1.53 | R62432 | Hs.439909 | SLC24A3 |
| 586 | 208893_s_at | 1957.72 | 3.54 | 1.33 | 1.59 | BC005047 | Hs.298654 | DUSP6 |
| 587 | 210754_s_at | 1960.94 | 3.56 | 1.32 | 1.59 | M79321 | Hs.80887 | LYN |
| 588 | 215113_s_at | 1961.19 | 3.56 | 1.34 | 1.62 | AK000923 | Hs.255022 | SENP3 |
| 589 | 212792_at | 1961.43 | 3.55 | 1.34 | 1.62 | AB020684 | Hs.408623 | KIAA0877 |
| 590 | 203110_at | 1962.52 | 3.55 | 1.33 | 1.60 | U43522 | Hs.405474 | PTK2B |
| 591 | 206157_at | 1963.06 | 3.55 | 1.19 | 1.34 | NM_002852 | Hs.2050 | PTX3 |
| 592 | 222088_s_at | 1964.81 | 3.57 | 1.31 | 1.56 | AA778684 | Hs.401274 | SLC2A14 /// |
| | | | | | | | | SLC2A3 |
| 593 | 202468_s_at | 1967.82 | 3.59 | 1.30 | 1.55 | NM_003798 | Hs.58488 | CTNNAL1 |
| 594 | 209042_s_at | 1968.26 | 3.59 | 1.31 | 1.56 | BC001738 | Hs.250387 | UBE2G2 |
| 595 | 208776_at | 1969.42 | 3.59 | 1.34 | 1.63 | BF432873 | Hs.443379 | PSMD11 |
| 596 | 200641_s_at | 1971.23 | 3.61 | 1.29 | 1.52 | U28964 | Hs.386834 | YWHAZ |
| 597 | 220751_s_at | 1971.40 | 3.61 | 1.27 | 1.47 | NM_016348 | Hs.10235 | C5orf4 |
| 598 | 217771_at | 1971.97 | 3.61 | 1.33 | 1.59 | NM_016548 | Hs.352662 | GOLPH2 |
| 599 | 222311_s_at | 1973.46 | 3.62 | 1.33 | 1.60 | AA648521 | Hs.125134 | SFRS15 |
| 600 | 221484_at | 1973.75 | 3.62 | 1.22 | 1.39 | BF691447 | Hs.107526 | B4GALT5 |
| 601 | 201059_at | 1976.41 | 3.64 | 1.28 | 1.50 | NM_005231 | Hs.301348 | CTTN |
| 602 | 201078_at | 1983.01 | 3.72 | 1.33 | 1.60 | NM_004800 | Hs.298272 | TM9SF2 |
| 603 | 212206_s_at | 1984.00 | 3.72 | 1.34 | 1.62 | BF343852 | Hs.301005 | H2AFV |
| 604 | 217882_at | 1984.52 | 3.72 | 1.23 | 1.40 | NM_018447 | Hs.444846 | LOC55831 |
| 605 | 200897_s_at | 1985.37 | 3.72 | 1.24 | 1.43 | NM_016081 | Hs.194431 | KIAA0992 |
| 606 | 200624_s_at | 1985.93 | 3.72 | 1.34 | 1.62 | AA577695 | Hs.223745 | MATR3 |
| 607 | 202084_s_at | 1986.52 | 3.72 | 1.33 | 1.59 | NM_003003 | Hs.75232 | SEC14L1 |
| 608 | 201916_s_at | 1989.53 | 3.75 | 1.33 | 1.59 | NM_007214 | Hs.330767 | SEC63 |
| 609 | 207238_s_at | 1990.32 | 3.74 | 1.35 | 1.64 | NM_002838 | Hs.444324 | PTPRC |
| 610 | 221677_s_at | 1992.04 | 3.75 | 1.33 | 1.59 | AF232674 | — | — |
| 611 | 206710 s_at | 1994.27 | 3.77 | 1.33 | 1.59 | NM_012307 | Hs.103839 | EPB41L3 |
| 612 | 219359_at | 1995.54 | 3.78 | 1.29 | 1.52 | NM_025092 | Hs.353181 | FLJ22635 |
| 613 | 204924_at | 1995.66 | 3.77 | 1.30 | 1.55 | NM_003264 | Hs.519033 | TLR2 |
| 614 | 208894_at | 2000.16 | 3.82 | 1.29 | 1.51 | M60334 | Hs.409805 | HLA-DRA |
| 615 | 203196_at | 2001.33 | 3.82 | 1.30 | 1.53 | AI948503 | Hs.307915 | ABCC4 |
| 616 | 202761_s_at | 2004.93 | 3.84 | 1.21 | 1.37 | NM_015180 | Hs.444069 | SYNE2 |
| 617 | 218940_at | 2006.27 | 3.85 | 1.30 | 1.55 | NM_024558 | Hs.390504 | C14orf138 |
| 618 | 201850_at | 2009.51 | 3.88 | 1.28 | 1.49 | NM_001747 | Hs.82422 | CAPG |
| 619 | 221781_s_at | 2011.87 | 3.90 | 1.33 | 1.60 | BG168666 | Hs.1098 | DNAJC10 |
| 620 | 218343_s_at | 2016.03 | 3.94 | 1.34 | 1.62 | NM_012086 | Hs.512838 | GTF3C3 |
| 621 | 205627_at | 2016.33 | 3.93 | 1.32 | 1.57 | NM_001785 | Hs.72924 | CDA |
| 622 | 202902_s_at | 2017.08 | 3.93 | 1.32 | 1.58 | NM_004079 | Hs.181301 | CTSS |
| 623 | 201735_s_at | 2017.66 | 3.93 | 1.28 | 1.49 | NM_001829 | Hs.372528 | CLCN3 |
| 624 | 204715_at | 2017.81 | 3.93 | 1.32 | 1.58 | NM_015368 | Hs.32163 | PANX1 |
| 625 | 201321_s_at | 2019.57 | 3.94 | 1.34 | 1.62 | NM_003075 | Hs.236030 | SMARCC2 |
| 626 | 200613_at | 2019.58 | 3.94 | 1.31 | 1.55 | NM_004068 | Hs.433892 | AP2M1 |
| 627 | 203485_at | 2020.72 | 3.94 | 1.30 | 1.53 | NM_021136 | Hs.99947 | RTN1 |
| 628 | 217742_s_at | 2021.10 | 3.93 | 1.32 | 1.58 | NM_016628 | Hs.370152 | WAC |
| 629 | 215240_at | 2023.06 | 3.95 | 1.16 | 1.28 | AI189839 | Hs.87149 | ITGB3 |
| 630 | 212828_at | 2023.51 | 3.94 | 1.32 | 1.57 | AA191573 | Hs.434494 | SYNJ2 |
| 631 | 201504_s_at | 2027.18 | 3.99 | 1.32 | 1.58 | AI435302 | Hs.75066 | TSN |
| 632 | 211075_s_at | 2028.19 | 3.99 | 1.30 | 1.55 | Z25521 | Hs.446414 | CD47 |
| 633 | 217100_s_at | 2028.57 | 3.99 | 1.33 | 1.60 | AK026451 | Hs.127287 | KIAA0794 |
| 634 | 205221_at | 2031.06 | 4.01 | 1.17 | 1.30 | NM_000187 | Hs.116992 | HGD |
| 635 | 215252_at | 2036.44 | 4.05 | 1.32 | 1.57 | AW814026 | — | — |
| 636 | 212227_x_at | 2036.54 | 4.05 | 1.32 | 1.57 | W67644 | Hs.150580 | SUI1 |
| 637 | 210101_x_at | 2036.71 | 4.04 | 1.30 | 1.54 | AF257318 | Hs.136309 | SH3GLB1 |
| 638 | 210988_s_at | 2039.15 | 4.07 | 1.32 | 1.58 | AF123538 | — | HTCD37 |
| 639 | 203471_s_at | 2039.25 | 4.07 | 1.30 | 1.54 | NM_002664 | Hs.77436 | PLEK |
| 640 | 211085_s_at | 2041.53 | 4.09 | 1.30 | 1.54 | Z25430 | Hs.35140 | STK4 |
| 641 | 201926_s_at | 2042.73 | 4.11 | 1.31 | 1.56 | BC001288 | Hs.408864 | DAF |
| 642 | 39318_at | 2042.79 | 4.10 | 1.11 | 1.19 | X82240 | Hs.2484 | TCL1A |
| 643 | 210269_s_at | 2046.95 | 4.14 | 1.32 | 1.58 | M99578 | — | DXYS155E |
| 644 | 207564_x_at | 2049.68 | 4.16 | 1.33 | 1.60 | NM_003605 | Hs.405410 | OGT |
| 645 | 221381_s_at | 2050.29 | 4.17 | 1.30 | 1.54 | NM_006792 | Hs.374503 | MORF4L1 |
| 646 | 213465_s_at | 2054.91 | 4.22 | 1.30 | 1.54 | BF718769 | Hs.36587 | PPP1R7 |
| 647 | 212721_at | 2055.41 | 4.21 | 1.32 | 1.58 | AI810380 | Hs.371166 | SFRS12 |
| 648 | 202268_s_at | 2057.04 | 4.22 | 1.33 | 1.59 | NM_003905 | Hs.418162 | APPBP1 |
| 649 | 200898_s_at | 2059.51 | 4.24 | 1.32 | 1.58 | AK002091 | Hs.5734 | MGEA5 |
| 650 | 200710_at | 2065.31 | 4.30 | 1.30 | 1.54 | NM_000018 | Hs.437178 | ACADVL |
| 651 | 202021_x_at | 2067.54 | 4.31 | 1.30 | 1.54 | AF083441 | Hs.150580 | SUI1 |
| 652 | 212070_at | 2068.94 | 4.33 | 1.25 | 1.43 | AL554008 | Hs.6527 | GPR56 |
| 653 | 201520_s_at | 2069.55 | 4.33 | 1.31 | 1.56 | BF034561 | Hs.309763 | GRSF1 |
| 654 | 212301_at | 2070.32 | 4.33 | 1.34 | 1.62 | D87440 | Hs.83419 | KIAA0252 |
| 655 | 217078_s_at | 2070.44 | 4.32 | 1.30 | 1.53 | AJ010102 | Hs.9688 | CMRF-35H |
| 656 | 202162_s_at | 2070.94 | 4.33 | 1.33 | 1.60 | AI769416 | Hs.26703 | CNOT8 |
| 657 | 218340_s_at | 2071.68 | 4.33 | 1.25 | 1.45 | NM_018227 | Hs.59838 | FLJ10808 |
| 658 | 206383_s_at | 2072.13 | 4.32 | 1.32 | 1.58 | NM_012297 | Hs.303676 | G3BP2 |
| 659 | 216940_x_at | 2074.96 | 4.35 | 1.29 | 1.51 | X96666 | — | — |
| 660 | 213142_x_at | 2075.62 | 4.35 | 1.27 | 1.48 | AV700415 | Hs.186649 | LOC54103 |
| 661 | 214241_at | 2076.14 | 4.35 | 1.32 | 1.58 | AA723057 | Hs.198273 | NDUFB8 |
| 662 | 203311_s_at | 2077.63 | 4.35 | 1.32 | 1.58 | M57763 | Hs.89474 | ARF6 |
| 663 | 218191_s_at | 2079.64 | 4.36 | 1.31 | 1.56 | NM_018368 | Hs.439494 | C6orf209 |
| 664 | 204255_s_at | 2080.36 | 4.36 | 1.26 | 1.46 | AA772285 | Hs.2062 | VDR |
| 665 | 220122_at | 2084.31 | 4.39 | 1.04 | 1.06 | NM_024717 | Hs.107716 | MCTP1 |
| 666 | 212008_at | 2085.70 | 4.40 | 1.33 | 1.60 | N29889 | Hs.350806 | UBXD2 |
| 667 | 213361_at | 2087.72 | 4.40 | 1.32 | 1.57 | AW129593 | Hs.416543 | TDRD7 |
| 668 | 203519_s_at | 2088.37 | 4.41 | 1.32 | 1.58 | NM_015542 | Hs.3862 | UPF2 |
| 669 | 201696_at | 2088.94 | 4.41 | 1.33 | 1.59 | NM_005626 | Hs.76122 | SFRS4 |
| 670 | 203670_at | 2089.47 | 4.41 | 1.30 | 1.53 | NM_015644 | Hs.511982 | TTLL3 |
| 671 | 211317_s_at | 2089.80 | 4.41 | 1.30 | 1.54 | AF041461 | Hs.355724 | CFLAR |
| 672 | 206414_s_at | 2090.37 | 4.40 | 1.22 | 1.38 | NM_003887 | Hs.12802 | DDEF2 |
| 673 | 211862_x_at | 2090.42 | 4.40 | 1.31 | 1.56 | AF015451 | Hs.355724 | CFLAR |
| 674 | 217356_s_at | 2092.22 | 4.43 | 1.29 | 1.53 | S81916 | Hs.78771 | PGK1 |
| 675 | 204589_at | 2093.87 | 4.43 | 1.36 | 1.65 | NM_014840 | Hs.200598 | ARK5 |
| 676 | 221641_s_at | 2093.89 | 4.42 | 1.33 | 1.60 | AF241787 | Hs.298885 | ACATE2 |
| 677 | 205568_at | 2094.05 | 4.42 | 1.23 | 1.40 | NM_020980 | Hs.104624 | AQP9 |
| 678 | 212135_s_at | 2054.65 | 4.42 | 1.30 | 1.54 | AW517686 | Hs.343522 | ATP2B4 |
| 679 | 209993_at | 2097.47 | 4.45 | 1.32 | 1.58 | AF016535 | Hs.21330 | ABCB1 |
| 680 | 214752_x_at | 2097.80 | 4.45 | 1.30 | 1.54 | AI625550 | Hs.195464 | FLNA |
| 681 | 204581_at | 2100.76 | 4.47 | 1.31 | 1.55 | NM_001771 | Hs.262150 | CD22 |
| 682 | 205241_at | 2102.77 | 4.49 | 1.12 | 1.20 | NM_005138 | Hs.410944 | SCO2 |
| 683 | 206828_at | 2102.96 | 4.49 | 1.26 | 1.46 | NM_003328 | Hs.29877 | TXK |
| 684 | 217795_s_at | 2104.64 | 4.50 | 1.33 | 1.60 | W74580 | Hs.130330 | MGC3222 |
| 685 | 203561_at | 2105.39 | 4.50 | 1.29 | 1.51 | NM_021642 | Hs.352642 | FCGR2A |
| 686 | 222045_s_at | 2107.58 | 4.52 | 1.27 | 1.49 | AI199589 | Hs.272814 | C20orf67 |
| 687 | 219600_s_at | 2108.63 | 4.53 | 1.31 | 1.56 | NM_006134 | Hs.433668 | C21orf4 |
| 688 | 202887_s_at | 2108.77 | 4.53 | 1.25 | 1.44 | NM_019058 | Hs.111244 | DDIT4 |
| 689 | 209514_s_at | 2110.27 | 4.54 | 1.31 | 1.55 | BE502030 | Hs.298530 | RAB27A |
| 690 | 202104_s_at | 2111.63 | 4.54 | 1.30 | 1.55 | NM_003119 | Hs.311765 | SPG7 |
| 691 | 209287_s_at | 2112.69 | 4.55 | 1.31 | 1.56 | AF104857 | Hs.352554 | CDC42EP3 |
| 692 | 212753_at | 2113.01 | 4.54 | 1.27 | 1.48 | AI692203 | Hs.435065 | RNF3 |
| 693 | 203241_at | 2113.34 | 4.55 | 1.33 | 1.59 | NM_003369 | Hs.13137 | UVRAG |
| 694 | 216261_at | 2115.51 | 4.56 | 1.21 | 1.36 | AI151479 | Hs.87149 | ITGB3 |
| 695 | 212599_at | 2118.12 | 4.58 | 1.34 | 1.62 | AK025298 | Hs.296720 | AUTS2 |
| 696 | 211160_x_at | 2119.87 | 4.59 | 1.31 | 1.56 | M95178 | Hs.119000 | ACTN1 |
| 697 | 207306_at | 2126.77 | 4.67 | 1.31 | 1.55 | NM_004609 | Hs.437 | TCF15 |
| 698 | 201473_at | 2127.44 | 4.68 | 1.24 | 1.42 | NM_002229 | Hs.25292 | JUNB |
| 699 | 201931_at | 2128.63 | 4.68 | 1.27 | 1.48 | NM_000126 | Hs.169919 | ETFA |
| 700 | 208922_s_at | 2128.87 | 4.67 | 1.31 | 1.56 | BC004904 | Hs.323502 | NXF1 |
| 701 | 210024_s_at | 2130.41 | 4.69 | 1.29 | 1.52 | AB017644 | Hs.449501 | UBE2E3 |
| 702 | 208869_s_at | 2131.33 | 4.69 | 1.27 | 1.48 | AF087847 | Hs.336429 | GABARAPL1 |
| 703 | 204286_s_at | 2131.55 | 4.69 | 1.22 | 1.38 | NM_021127 | Hs.96 | PMAIP1 |
| 704 | 211986_at | 2132.41 | 4.70 | 1.32 | 1.57 | BG287862 | Hs.378738 | AHNAK |
| 705 | 208854_s_at | 2132.96 | 4.70 | 1.32 | 1.57 | AA586774 | Hs.168913 | STK24 |
| 706 | 212124_at | 2137.17 | 4.74 | 1.31 | 1.56 | AF070622 | Hs.438767 | RAI17 |
| 707 | 208627_s_at | 2137.29 | 4.74 | 1.31 | 1.55 | BE966374 | Hs.74497 | NSEP1 |
| 708 | 217736_s_at | 2140.83 | 4.78 | 1.29 | 1.53 | NM_014413 | Hs.434986 | HRI |
| 709 | 210563_x_at | 2144.88 | 4.83 | 1.30 | 1.55 | U97075 | Hs.355724 | CFLAR |
| 710 | 218032_at | 2145.77 | 4.83 | 1.25 | 1.43 | AF070673 | Hs.76691 | SNN |
| 711 | 218348_s_at | 2147.67 | 4.84 | 1.33 | 1.59 | NM_014153 | Hs.371856 | ZC3HDC7 |
| 712 | 212888_at | 2148.81 | 4.85 | 1.32 | 1.57 | BG109746 | Hs.87889 | DICER1 |
| 713 | 218041_x_at | 2150.44 | 4.86 | 1.32 | 1.57 | NM_018573 | Hs.298275 | SLC38A2 |
| 714 | 208579_x_at | 2150.97 | 4.86 | 1.18 | 1.30 | NM_017445 | Hs.247817 | HIST1N2BK |
| 715 | 212256_at | 2152.10 | 4.86 | 1.31 | 1.55 | BE906572 | Hs.13785 | GALNT10 |
| 716 | 208161_s_at | 2152.15 | 4.86 | 1.27 | 1.48 | NM_020037 | Hs.90786 | ABCC3 |
| 717 | 210235_s_at | 2153.99 | 4.88 | 1.30 | 1.54 | U22815 | Hs.128312 | PPFIA1 |
| 718 | 211797_s_at | 2156.02 | 4.91 | 1.32 | 1.58 | U62296 | Hs.285133 | NFYC |
| 719 | 212131_at | 2157.53 | 4.92 | 1.32 | 1.58 | BG054966 | Hs.8258 | C19orf13 |
| 720 | 201132_at | 2157.70 | 4.91 | 1.32 | 1.58 | NM_019597 | Hs.278857 | HNRPH2 |
| 721 | 202771_at | 2164.41 | 5.00 | 1.28 | 1.50 | NM_014745 | Hs.79077 | FAM38A |
| 722 | 204562_at | 2165.54 | 5.01 | 1.30 | 1.55 | NM_002460 | Hs.127686 | IRF4 |
| 723 | 215424_s_at | 2168.39 | 5.05 | 1.31 | 1.56 | AV689564 | Hs.445498 | SKIIP |
| 724 | 209688_s_at | 2169.79 | 5.06 | 1.31 | 1.57 | BC005078 | Hs.98324 | FLJ10996 |
| 725 | 221834_at | 2171.37 | 5.07 | 1.31 | 1.55 | AV700132 | Hs.301872 | LONP |
| 726 | 209806_at | 2172.42 | 5.07 | 1.20 | 1.35 | BC000893 | Hs.247817 | HIST1H2BK |
| 727 | 212240_s_at | 2174.14 | 5.09 | 1.31 | 1.56 | AI679268 | Hs.6241 | PIK3R1 |
| 728 | 210371_s_at | 2176.37 | 5.10 | 1.31 | 1.56 | BC003092 | Hs.16003 | RBBP4 |
| 729 | 211794_at | 2176.53 | 5.10 | 1.16 | 1.27 | AF198052 | Hs.276506 | FYB |
| 730 | 211864_s_at | 2178.30 | 5.11 | 1.23 | 1.41 | AF207990 | Hs.362731 | FER1L3 |
| 731 | 203086_at | 2178.86 | 5.10 | 1.26 | 1.47 | BE872563 | Hs.113319 | KIF2 |
| 732 | 213018_at | 2179.68 | 5.11 | 1.31 | 1.55 | AI337901 | Hs.21145 | ODAG |
| 733 | 204007_at | 2182.99 | 5.16 | 1.24 | 1.42 | J04162 | — | FCGR3A |
| 734 | 215908_at | 2183.38 | 5.15 | 1.21 | 1.36 | AF009267 | — | — |
| 735 | 208698_s_at | 2183.46 | 5.15 | 1.31 | 1.56 | L14599 | Hs.355861 | NONO |
| 736 | 212202_s_at | 2183.93 | 5.15 | 1.32 | 1.58 | BG493972 | Hs.200692 | DKFZP564G2022 |
| 737 | 202061_s_at | 2184.19 | 5.15 | 1.32 | 1.58 | AI927770 | Hs.181300 | SEL1L |
| 738 | 202654_x_at | 2184.59 | 5.15 | 1.31 | 1.55 | NM_022826 | Hs.5306 | AXOT |
| 739 | 221972_s_at | 2184.70 | 5.15 | 1.32 | 1.57 | AL571362 | Hs.42806 | Cab45 |
| 740 | 205548_s_at | 2184.82 | 5.14 | 1.31 | 1.55 | NM_006806 | Hs.77311 | BTG3 |
| 741 | 220439_at | 2185.27 | 5.14 | 1.32 | 1.58 | NM_024892 | Hs.413374 | RIN3 |
| 742 | 211015_s_at | 2185.37 | 5.13 | 1.31 | 1.56 | L12723 | Hs.90093 | HSPA4 |
| 743 | 222044_at | 2185.87 | 5.13 | 1.26 | 1.46 | AI199589 | Hs.272814 | C20orf67 |
| 744 | 201693_s_at | 2186.62 | 5.14 | 1.02 | 1.04 | AV733950 | Hs.326035 | EGR1 |
| 745 | 206465_at | 2186.92 | 5.14 | 1.23 | 1.40 | BE856376 | — | — |
| 746 | 204197_s_at | 2187.32 | 5.14 | 1.31 | 1.56 | NM_004350 | Hs.170019 | RUNX3 |
| 747 | 200666_s_at | 2187.47 | 5.13 | 1.30 | 1.54 | NM_006145 | Hs.82646 | DNAJB1 |
| 748 | 219708_at | 2190.22 | 5.15 | 1.26 | 1.46 | NM_020201 | Hs.16614 | NT5M |
| 749 | 221676_s_at | 2194.28 | 5.20 | 1.24 | 1.42 | BC002342 | Hs.17377 | CORO1C |
| 750 | 218880_at | 2194.65 | 5.20 | 1.29 | 1.53 | N36408 | Hs.301612 | FOSL2 |
| 751 | 204150_at | 2194.72 | 5.19 | 1.27 | 1.47 | NM_015136 | Hs.301989 | STAB1 |
| 752 | 204644_at | 2195.63 | 5.19 | 1.30 | 1.53 | AF207881 | Hs.155185 | COVA1 |
| 753 | 206371_at | 2198.61 | 5.23 | 1.43 | 1.80 | NM_000804 | Hs.352 | FOLR3 |
| 754 | 204376_at | 2199.10 | 5.23 | 1.29 | 1.52 | NM_014703 | Hs.118738 | VprBP |
| 755 | 207677_s_at | 2199.34 | 5.22 | 1.29 | 1.53 | NM_013416 | Hs.196352 | NCF4 |
| 756 | 213988_s_at | 2200.93 | 5.23 | 1.15 | 1.26 | BE971383 | Hs.28491 | SAT |
| 757 | 203665_at | 2201.40 | 5.23 | 1.20 | 1.34 | NM_002133 | Hs.202833 | HMOX1 |
| 758 | 209027_s_at | 2201.52 | 5.23 | 1.31 | 1.57 | BF673013 | Hs.42710 | ABI1 |
| 759 | 207198_s_at | 2203.14 | 5.24 | 1.20 | 1.35 | NM_004987 | Hs.112378 | LIMS1 |
| 760 | 209180_at | 2205.45 | 5.25 | 1.33 | 1.59 | U49245 | Hs.78948 | RABGGTB |
| 761 | 211068_x_at | 2206.73 | 5.26 | 1.31 | 1.55 | BC006456 | Hs.439367 | KIAA0592 |
| 762 | 202380_s_at | 2210.28 | 5.31 | 1.32 | 1.58 | NM_005385 | Hs.369815 | NKTR |
| 763 | 204019_s_at | 2210.47 | 5.30 | 1.28 | 1.50 | NM_015677 | Hs.147365 | SH3YL1 |
| 764 | 206254_at | 2210.75 | 5.30 | 1.25 | 1.44 | NM_001963 | Hs.419815 | EGF |
| 765 | 214769_at | 2212.56 | 5.31 | 1.28 | 1.49 | AF052117 | Hs.417091 | CLCN4 |
| 766 | 217827_s_at | 2213.91 | 5.32 | 1.30 | 1.55 | NM_016630 | Hs.242458 | SPG21 |
| 767 | 218017_s_at | 2215.58 | 5.33 | 1.33 | 1.60 | NM_025070 | Hs.191320 | FLJ32731 |
| 768 | 201669_s_at | 2216.16 | 5.34 | 1.27 | 1.48 | NM_002356 | Hs.318603 | MARCKS |
| 769 | 213140_s_at | 2217.16 | 5.35 | 1.31 | 1.56 | AB014593 | Hs.154429 | SS18L1 |
| 770 | 208924_at | 2218.04 | 5.35 | 1.27 | 1.48 | AB024703 | Hs.96334 | RNF11 |
| 771 | 203674_at | 2218.47 | 5.35 | 1.31 | 1.56 | NM_014877 | Hs.99437 | HELZ |
| 772 | 210653_s_at | 2218.68 | 5.34 | 1.30 | 1.55 | M55575 | Hs.1265 | BCKDHB |
| 773 | 210757_x_at | 2220.78 | 5.35 | 1.22 | 1.38 | AF188298 | Hs.81988 | DAB2 |
| 774 | 213792_s_at | 2222.50 | 5.36 | 1.28 | 1.50 | AA485908 | Hs.438669 | INSR |
| 775 | 215375_x_at | 2222.97 | 5.36 | 1.28 | 1.51 | AK023938 | Hs.438377 | — |
| 776 | 208885_at | 2223.91 | 5.36 | 1.29 | 1.53 | J02923 | Hs.381099 | LCP1 |
| 777 | 202378_s_at | 2223.92 | 5.36 | 1.25 | 1.44 | NM_017526 | — | OBRGRP |
| 778 | 203710_at | 2226.32 | 5.38 | 1.33 | 1.59 | NM_002222 | Hs.149900 | ITPR1 |
| 779 | 204982_at | 2228.30 | 5.40 | 1.30 | 1.53 | NM_014776 | Hs.418057 | GIT2 |
| 780 | 210356_x_at | 2232.25 | 5.43 | 1.29 | 1.53 | BC002807 | Hs.438040 | MS4A1 |
| 781 | 216033_s_at | 2232.64 | 5.43 | 1.31 | 1.57 | S74774 | Hs.390567 | FYN |
| 782 | 202478_at | 2233.64 | 5.43 | 1.31 | 1.56 | NM_021643 | Hs.155418 | TRIB2 |
| 783 | 201844_s_at | 2233.84 | 5.42 | 1.28 | 1.49 | W84482 | Hs.7910 | RYBP |
| 784 | 205681_at | 2238.59 | 5.47 | 1.21 | 1.37 | NM_004049 | Hs.227817 | BCL2A1 |
| 785 | 217825_s_at | 2240.55 | 5.49 | 1.30 | 1.53 | AF151039 | Hs.184325 | UBE2J1 |
| 786 | 202862_at | 2241.55 | 5.49 | 1.25 | 1.44 | NM_000137 | Hs.73875 | FAH |
| 787 | 218172_s_at | 2242.30 | 5.49 | 1.29 | 1.52 | NM_018630 | Hs.241576 | DER1 |
| 788 | 221853_s_at | 2244.90 | 5.51 | 1.27 | 1.48 | N39536 | Hs.227823 | NOMO2 |
| 789 | 204203_at | 2245.81 | 5.51 | 1.27 | 1.47 | NM_001806 | Hs.2227 | CEBPG |
| 790 | 203949_at | 2246.84 | 5.51 | 1.30 | 1.54 | NM_000250 | — | — |
| 791 | 217748_at | 2248.39 | 5.52 | 1.29 | 1.52 | NM_015999 | Hs.5298 | ADIPOR1 |
| 792 | 219714_s_at | 2249.51 | 5.53 | 1.30 | 1.54 | NM_018398 | — | — |
| 793 | 202606_s_at | 2250.15 | 5.53 | 1.29 | 1.52 | NM_012290 | Hs.369280 | TLK1 |
| 794 | 213047_x_at | 2250.72 | 5.53 | 1.27 | 1.48 | AI278616 | Hs.436687 | SET |
| 795 | 213839_at | 2255.29 | 5.60 | 1.31 | 1.55 | AW028110 | — | KIAA0500 |
| 796 | 205953_at | 2255.44 | 5.59 | 1.29 | 1.53 | NM_014813 | Hs.448972 | LRIG2 |
| 797 | 201889_at | 2255.59 | 5.59 | 1.31 | 1.56 | NM_014888 | Hs.434053 | FAM3C |
| 798 | 222235_s_at | 2258.12 | 5.62 | 1.33 | 1.59 | AL139812 | Hs.180758 | GALNACT-2 |
| 799 | 210555_s_at | 2259.84 | 5.64 | 1.31 | 1.56 | U85430 | Hs.172674 | NFATC3 |
| 800 | 203411_s_at | 2260.50 | 5.65 | 1.30 | 1.53 | NM_005572 | Hs.436441 | LMNA |
| 801 | 201280_s_at | 2264.17 | 5.70 | 1.20 | 1.36 | NM_001343 | Hs.81988 | DAB2 |
| 802 | 202510_s_at | 2264.28 | 5.69 | 1.24 | 1.43 | NM_006291 | Hs.101382 | TNFAIP2 |
| 803 | 210785_s_at | 2265.96 | 5.71 | 1.31 | 1.56 | AB035482 | Hs.10649 | C1orf38 |
| 804 | 202615_at | 2267.03 | 5.72 | 1.30 | 1.53 | BF222895 | Hs.380144 | — |
| 805 | 212031_at | 2268.07 | 5.74 | 1.28 | 1.50 | AV757384 | Hs.197184 | RBM25 |
| 806 | 202168_at | 2268.78 | 5.74 | 1.32 | 1.58 | NM_003187 | Hs.60679 | TAF9 |
| 807 | 201425_at | 2269.50 | 5.74 | 1.31 | 1.56 | NM_000690 | Hs.331141 | ALDH2 |
| 808 | 216983_s_at | 2269.69 | 5.74 | 1.29 | 1.52 | BC002889 | Hs.279855 | ZNF224 |
| 809 | 203675_at | 2269.95 | 5.73 | 1.27 | 1.48 | NM_005013 | Hs.423095 | NUCB2 |
| 810 | 203258_at | 2274.05 | 5.77 | 1.29 | 1.53 | NM_006442 | Hs.356742 | DRAP1 |
| 811 | 212460_at | 2274.19 | 5.76 | 1.29 | 1.52 | BE738425 | Hs.353161 | C14orf147 |
| 812 | 208406_s_at | 2274.85 | 5.76 | 1.24 | 1.42 | NM_004810 | Hs.193076 | GRAP2 |
| 813 | 218035_s_at | 2276.34 | 5.77 | 1.28 | 1.51 | NM_019027 | Hs.95549 | FLJ20273 |
| 814 | 220702_at | 2278.36 | 5.79 | 1.29 | 1.52 | NM_018616 | — | — |
| 815 | 213817_at | 2278.72 | 5.79 | 1.23 | 1.40 | AL049435 | Hs.407903 | — |
| 816 | 201586_s_at | 2279.07 | 5.78 | 1.30 | 1.54 | NM_005066 | — | — |
| 817 | 202501_at | 2281.37 | 5.82 | 1.30 | 1.53 | NM_014268 | Hs.446375 | MAPRE2 |
| 818 | 201711_x_at | 2284.74 | 5.85 | 1.25 | 1.45 | AI681120 | Hs.199179 | RANBP2 |
| 819 | 216988_s_at | 2286.23 | 5.87 | 1.29 | 1.53 | L48722 | Hs.82911 | PTP4A2 |
| 820 | 215043_s_at | 2286.69 | 5.86 | 1.27 | 1.48 | X83301 | Hs.166361 | SMA5 |
| 821 | 201417_at | 2287.47 | 5.87 | 1.22 | 1.38 | AL136179 | Hs.357901 | SOX4 |
| 822 | 202777_at | 2288.04 | 5.87 | 1.31 | 1.56 | NM_007373 | Hs.104315 | SHOC2 |
| 823 | 200742_s_at | 2290.07 | 5.89 | 1.29 | 1.52 | BG231932 | Hs.429658 | CLN2 |
| 824 | 204546_at | 2290.59 | 5.89 | 1.28 | 1.49 | NM_014732 | Hs.301658 | KIAA0513 |
| 825 | 202165_at | 2291.12 | 5.89 | 1.31 | 1.55 | BF966540 | Hs.267819 | PPP1R2 |
| 826 | 216652_s_at | 2293.65 | 5.92 | 1.30 | 1.53 | AL137673 | Hs.348418 | DR1 |
| 827 | 200698_at | 2293.65 | 5.91 | 1.31 | 1.56 | AL542253 | Hs.446645 | KDELR2 |
| 828 | 209994_s_at | 2293.82 | 5.91 | 1.29 | 1.51 | AF016535 | Hs.21330 | ABCB1 /// ABCB4 |
| 829 | 206697_s_at | 2294.07 | 5.91 | 1.29 | 1.51 | NM_005143 | Hs.403931 | HP |
| 830 | 203139_at | 2294.88 | 5.90 | 1.24 | 1.42 | NM_004938 | Hs.244318 | DAPK1 |
| 831 | 212808_at | 2295.29 | 5.90 | 1.25 | 1.45 | AI884627 | Hs.334788 | FLJ14639 |
| 832 | 202587_s_at | 2295.30 | 5.90 | 1.23 | 1.41 | BC001116 | Hs.76240 | AK1 |
| 833 | 213208_at | 2295.37 | 5.89 | 1.31 | 1.57 | AI801951 | Hs.196275 | KIAA0240 |
| 834 | 200808_s_at | 2301.70 | 5.95 | 1.30 | 1.53 | NM_003461 | Hs.75873 | ZYX |
| 835 | 218184_at | 2303.67 | 5.97 | 1.31 | 1.55 | NM_020245 | Hs.102237 | TULP4 |
| 836 | 212705_x_at | 2306.53 | 6.00 | 1.29 | 1.52 | BF570210 | Hs.118463 | PNPLA2 |
| 837 | 214736_s_at | 2308.28 | 6.02 | 1.31 | 1.55 | BE898639 | Hs.183706 | ADD1 |
| 838 | 222065_s_at | 2313.20 | 6.10 | 1.30 | 1.53 | AI830227 | Hs.531027 | FLII |
| 839 | 201278_at | 2315.96 | 6.14 | 1.20 | 1.34 | N21202 | Hs.519270 /// | DAB2 |
| | | | | | | | Hs.81988 | |
| 840 | 203156_at | 2316.77 | 6.14 | 1.33 | 1.59 | NM_016248 | Hs.414995 | AKAP11 |
| 841 | 220034_at | 2316.86 | 6.14 | 1.23 | 1.40 | NM_007199 | Hs.268552 | IRAK3 |
| 842 | 200704_at | 2319.96 | 6.17 | 1.28 | 1.50 | AB034747 | Hs.76507 | LITAF |
| 843 | 205327_s_at | 2320.30 | 6.16 | 1.29 | 1.52 | NM_001616 | Hs.389846 | ACVR2 |
| 844 | 212373_at | 2320.64 | 6.16 | 1.31 | 1.56 | AW139179 | Hs.362733 | FEM1B |
| 845 | 200596_s_at | 2320.68 | 6.16 | 1.29 | 1.52 | BE614908 | Hs.389559 | EIF3S10 |
| 846 | 215946_x_at | 2323.09 | 6.18 | 1.29 | 1.51 | AL022324 | Hs.348935 | IGLL1 |
| 847 | 205321_at | 2323.81 | 6.18 | 1.23 | 1.41 | NM_001415 | Hs.480368 | EIF2S3 |
| 848 | 201506_at | 2323.87 | 6.18 | 1.28 | 1.50 | NM_000358 | Hs.421496 | TGFBI |
| 849 | 200692_s_at | 2325.06 | 6.18 | 1.30 | 1.54 | NM_004134 | Hs.184233 | HSPA9B |
| 850 | 212025_s_at | 2327.15 | 6.21 | 1.30 | 1.53 | BG421186 | Hs.531027 | FLII |
| 851 | 209689_at | 2327.61 | 6.21 | 1.30 | 1.54 | BC005078 | Hs.98324 | FLJ10996 |
| 852 | 202197_at | 2329.40 | 6.23 | 1.28 | 1.50 | NM_021090 | Hs.412833 | MTMR3 |
| 853 | 202990_at | 2330.37 | 6.24 | 1.28 | 1.49 | NM_002863 | Hs.282417 | PYGL |
| 854 | 201373_at | 2330.49 | 6.23 | 1.30 | 1.54 | NM_000445 | Hs.79706 | PLEC1 |
| 855 | 209785_s_at | 2333.18 | 6.27 | 1.28 | 1.50 | AF065214 | Hs.18858 | PLA2G4C |
| 856 | 210512_s_at | 2334.48 | 6.27 | 1.27 | 1.47 | AF022375 | Hs.73793 | VEGF |
| 857 | 200864_s_at | 2334.69 | 6.27 | 1.24 | 1.43 | NM_004663 | Hs.75618 | RAB11A |
| 858 | 204061_at | 2334.82 | 6.27 | 1.31 | 1.55 | NM_005044 | Hs.147996 | PRKX |
| 859 | 213222_at | 2335.99 | 6.27 | 1.29 | 1.51 | AL049593 | Hs.429643 | PLCB1 |
| 860 | 209352_s_at | 2336.76 | 6.27 | 1.28 | 1.49 | AB014600 | Hs.13999 | SIN3B |
| 861 | 211727_s_at | 2337.45 | 6.27 | 1.29 | 1.53 | BC005895 | Hs.436988 | COX11 |
| 862 | 201394_s_at | 2337.97 | 6.27 | 1.29 | 1.52 | U23946 | Hs.439480 | RBM5 |
| 863 | 209651_at | 2338.77 | 6.27 | 1.28 | 1.49 | BC001830 | Hs.25511 | TGFB1I1 |
| 864 | 215888_at | 2339.08 | 6.28 | 1.32 | 1.57 | AK026889 | Hs.168625 | APRIN |
| 865 | 208761_s_at | 2339.60 | 6.27 | 1.30 | 1.53 | U67122 | Hs.81424 | SUMO1 |
| 866 | 202374_s_at | 2340.41 | 6.28 | 1.30 | 1.55 | NM_012414 | Hs.197289 | RAB3-GAP150 |
| 867 | 212651_at | 2340.74 | 6.27 | 1.13 | 1.22 | AB018283 | Hs.15099 | RHOBTB1 |
| 868 | 217824_at | 2343.48 | 6.30 | 1.28 | 1.50 | AW500009 | Hs.184325 | UBE2J1 |
| 869 | 207760_s_at | 2343.65 | 6.30 | 1.28 | 1.50 | NM_006312 | Hs.287994 | NCOR2 |
| 870 | 208623_s_at | 2343.97 | 6.30 | 1.30 | 1.54 | J05021 | Hs.403997 | VIL2 |
| 871 | 209962_at | 2346.12 | 6.32 | 1.27 | 1.48 | M34986 | Hs.127826 | EPOR |
| 872 | 201549_x_at | 2348.02 | 6.34 | 1.30 | 1.53 | NM_006618 | Hs.143323 | JARID1B |
| 873 | 218218_at | 2348.56 | 6.34 | 1.31 | 1.56 | NM_018171 | Hs.370019 | DIP13B |
| 874 | 216915_s_at | 2349.13 | 6.34 | 1.00 | 0.99 | S69182 | Hs.62 | PTPN12 |
| 875 | 201736_s_at | 2351.46 | 6.36 | 1.28 | 1.50 | BF000409 | Hs.380875 | TEB4 |
| 876 | 219549_s_at | 2352.35 | 6.37 | 1.26 | 1.46 | NM_006054 | Hs.404434 | RTN3 |
| 877 | 213532_at | 2354.59 | 6.39 | 1.29 | 1.52 | AI797833 | Hs.404914 | ADAM17 |
| 878 | 218781_at | 2355.96 | 6.41 | 1.31 | 1.55 | NM_024624 | Hs.424559 | SMC6L1 |
| 879 | 202386_s_at | 2356.23 | 6.40 | 1.30 | 1.54 | NM_019081 | Hs.432741 | LKAP |
| 880 | 213459_at | 2356.47 | 6.40 | 1.30 | 1.54 | AU155515 | Hs.433701 | RPL37A |
| 881 | 204724_s_at | 2356.73 | 6.39 | 1.25 | 1.44 | NM_001853 | Hs.126248 | COL9A3 |
| 882 | 200737_at | 2357.14 | 6.39 | 1.26 | 1.45 | NM_000291 | Hs.78771 | PGK1 |
| 883 | 212636_at | 2359.22 | 6.41 | 1.27 | 1.49 | AL031781 | Hs.153355 | QKI |
| 884 | 209607_x_at | 2360.81 | 6.43 | 1.28 | 1.51 | U08032 | — | SULT1A3 |
| 885 | 212268_at | 2362.39 | 6.45 | 1.29 | 1.52 | NM_030666 | Hs.381167 | SERPINB1 |
| 886 | 221580_s_at | 2363.34 | 6.45 | 1.29 | 1.51 | BC001972 | Hs.355750 | MGC5306 |
| 887 | 221849_s_at | 2366.01 | 6.48 | 1.29 | 1.53 | AA733079 | Hs.443636 | LOC90379 |
| 888 | 205603_s_at | 2366.74 | 6.48 | 1.28 | 1.50 | NM_007309 | — | DIAPH2 |
| 889 | 221211_s_at | 2367.99 | 6.50 | 0.98 | 0.97 | NM_020152 | Hs.41267 | C21orf7 |
| 890 | 213006_at | 2368.50 | 6.50 | 1.31 | 1.55 | AV655640 | — | CEBPD |
| 891 | 209907_s_at | 2369.07 | 6.50 | 1.30 | 1.53 | AF182198 | Hs.444708 | ITSN2 |
| 892 | 200706_s_at | 2369.93 | 6.50 | 1.29 | 1.52 | NM_004862 | Hs.76507 | LITAF |
| 893 | 219033_at | 2369.93 | 6.50 | 1.31 | 1.55 | NM_024615 | Hs.310185 | PARP8 |
| 894 | 217418_x_at | 2370.96 | 6.51 | 1.27 | 1.47 | X12530 | Hs.438040 | MS4A1 |
| 895 | 213089_at | 2371.94 | 6.52 | 1.28 | 1.50 | AU158490 | Hs.446379 | LOC153561 |
| 896 | 213379_at | 2372.21 | 6.52 | 1.25 | 1.45 | AF091086 | Hs.144304 | CL640 |
| 897 | 207389_at | 2373.01 | 6.52 | 1.26 | 1.46 | NM_000173 | Hs.1472 | GP1BA |
| 898 | 211650_x_at | 2374.60 | 6.53 | 1.31 | 1.56 | L34164 | Hs.413826 | IGHG1 |
| 899 | 202429_s_at | 2376.39 | 6.54 | 1.28 | 1.50 | AL353950 | Hs.272458 | PPP3CA |
| 900 | 210110_x_at | 2377.48 | 6.54 | 1.29 | 1.52 | AF132363 | Hs.156481 | HNRPH3 |
| 901 | 217664_at | 2380.58 | 6.59 | 1.29 | 1.52 | AA780524 | — | — |
| 902 | 204132_s_at | 2380.88 | 6.59 | 1.30 | 1.53 | NM_001455 | Hs.14845 | FOXO3A |
| 903 | 201887_at | 2381.12 | 6.58 | 1.21 | 1.37 | NM_001560 | Hs.285115 | IL13RA1 |
| 904 | 212735_at | 2382.47 | 6.59 | 1.28 | 1.50 | BF448041 | Hs.499355 | KIAA0226 |
| 905 | 206279_at | 2382.80 | 6.59 | 1.30 | 1.53 | NM_002760 | Hs.183165 | PRKY |
| 906 | 212263_at | 2383.45 | 6.59 | 1.29 | 1.52 | AI114716 | Hs.153355 | QKI |
| 907 | 210038_at | 2383.50 | 6.58 | 1.28 | 1.49 | AL137145 | Hs.408049 | PRKCQ |
| 908 | 221748_s_at | 2384.19 | 6.59 | 1.23 | 1.40 | AL046979 | Hs.439442 | TNS |
| 909 | 209307_at | 2384.46 | 6.59 | 1.25 | 1.44 | AB014540 | Hs.153026 | SWAP70 |
| 910 | 204526_s_at | 2387.72 | 6.61 | 1.27 | 1.47 | NM_007063 | Hs.442657 | TBC1D8 |
| 911 | 203922_s_at | 2388.82 | 6.62 | 1.26 | 1.45 | AI308863 | Hs.88974 | CYBB |
| 912 | 201462_at | 2389.40 | 6.63 | 1.28 | 1.50 | NM_014766 | Hs.75137 | SCRN1 |
| 913 | 215029_at | 2389.84 | 6.63 | 1.27 | 1.47 | AL117451 | Hs.293563 | — |
| 914 | 202988_s_at | 2390.37 | 6.63 | 1.29 | 1.52 | NM_002922 | Hs.75256 | RGS1 |
| 915 | 209092_s_at | 2390.41 | 6.62 | 1.30 | 1.53 | AF061730 | Hs.279061 | C17orf25 |
| 916 | 201301_s_at | 2391.25 | 6.62 | 1.28 | 1.51 | BC000182 | Hs.422986 | ANXA4 |
| 917 | 210474_s_at | 2392.45 | 6.64 | 1.30 | 1.54 | U04819 | Hs.192316 | CDC2L1 |
| 918 | 202129_s_at | 2393.19 | 6.64 | 1.29 | 1.52 | AW006290 | Hs.209061 | RIOK3 |
| 919 | 202252_at | 2394.01 | 6.65 | 1.23 | 1.40 | NM_002870 | Hs.151536 | RAB13 |
| 920 | 207782_s_at | 2394.85 | 6.65 | 1.28 | 1.50 | NM_007319 | Hs.3260 | PSEN1 |
| 921 | 205174_s_at | 2396.02 | 6.67 | 1.29 | 1.52 | NM_012413 | Hs.79033 | QPCT |
| 922 | 209015_s_at | 2396.33 | 6.66 | 1.26 | 1.46 | BC002446 | Hs.181195 | DNAJB6 |
| 923 | 208948_s_at | 2396.46 | 6.66 | 1.27 | 1.48 | BC000830 | Hs.6113 | STAU |
| 924 | 209970_x_at | 2400.77 | 6.72 | 1.28 | 1.50 | M87507 | Hs.2490 | CASP1 |
| 925 | 213836_s_at | 2401.11 | 6.72 | 1.28 | 1.49 | AW052084 | Hs.9398 | WIPI49 |
| 926 | 215222_x_at | 2403.12 | 6.74 | 1.29 | 1.52 | AK023406 | Hs.372463 | MACF1 |
| 927 | 208754_s_at | 2403.84 | 6.75 | 1.29 | 1.52 | AL162068 | Hs.419776 | NAP1L1 |
| 928 | 205329_s_at | 2404.21 | 6.75 | 1.28 | 1.50 | NM_003794 | Hs.267812 | SNX4 |
| 929 | 201554_x_at | 2407.21 | 6.79 | 1.17 | 1.28 | NM_004130 | Hs.174071 | GYG |
| 930 | 214107_x_at | 2407.29 | 6.78 | 1.27 | 1.49 | AW340850 | — | FLJ11822 |
| 931 | 201529_s_at | 2407.76 | 6.78 | 1.22 | 1.39 | NM_002945 | Hs.84318 | RPA1 |
| 932 | 214714_at | 2408.68 | 6.78 | 1.26 | 1.46 | AK022360 | Hs.386324 | ZNF394 |
| 933 | 220610_s_at | 2410.16 | 6.80 | 1.28 | 1.49 | NM_006309 | Hs.268768 | LRRFIP2 |
| 934 | 201543_s_at | 2410.89 | 6.81 | 1.27 | 1.49 | NM_020150 | Hs.110796 | SARA1 |
| 935 | 201627_s_at | 2412.11 | 6.82 | 1.28 | 1.50 | NM_005542 | Hs.416385 | INSIG1 |
| 936 | 222150_s_at | 2412.17 | 6.81 | 1.25 | 1.43 | AK026747 | Hs.186649 | LOC54103 |
| 937 | 221736_at | 2413.56 | 6.82 | 1.31 | 1.55 | AA156777 | Hs.348929 | KIAA1219 |
| 938 | 200764_s_at | 2414.30 | 6.83 | 1.28 | 1.51 | AI826881 | Hs.254321 | CTNNA1 |
| 939 | 210786_s_at | 2415.68 | 6.85 | 1.29 | 1.51 | M93255 | Hs.257049 | FLI1 |
| 940 | 201210_at | 2416.14 | 6.85 | 1.28 | 1.50 | NM_001356 | Hs.380774 | DDX3X |
| 941 | 203124_s_at | 2419.20 | 6.89 | 1.27 | 1.47 | NM_000617 | Hs.57435 | SLC11A2 |
| 942 | 207655_s_at | 2421.29 | 6.92 | 1.26 | 1.46 | NM_013314 | Hs.167746 | BLNK |
| 943 | 213145_at | 2421.35 | 6.91 | 1.30 | 1.53 | BF001666 | Hs.367956 | FBXL14 |
| 944 | 204849_at | 2421.76 | 6.92 | 1.24 | 1.41 | NM_006602 | Hs.30696 | TCFL5 |
| 945 | 200719_at | 2422.04 | 6.91 | 1.25 | 1.45 | BE964043 | Hs.171626 | SKP1A |
| 946 | 221920_s_at | 2423.59 | 6.92 | 1.25 | 1.45 | BE677761 | Hs.283716 | MSCP |
| 947 | 221432_s_at | 2423.68 | 6.92 | 1.28 | 1.49 | NM_031212 | Hs.326104 | SLC25A28 |
| 948 | 206700_s_at | 2424.73 | 6.93 | 1.28 | 1.51 | NM_004653 | Hs.80358 | JARID1D |
| 949 | 201583_s_at | 2425.02 | 6.92 | 1.26 | 1.46 | NM_006363 | Hs.173497 | SEC23B |
| 950 | 201061_s_at | 2425.45 | 6.92 | 1.25 | 1.44 | M81635 | Hs.439776 | STOM |
| 951 | 217388_s_at | 2425.56 | 6.91 | 1.26 | 1.47 | D55639 | Hs.444471 | KYNU |
| 952 | 210656_at | 2425.84 | 6.91 | 1.30 | 1.54 | AF099032 | Hs.512812 | EED |
| 953 | 213510_x_at | 2425.87 | 6.91 | 1.18 | 1.32 | AW194543 | Hs.234573 | LOC220594 |
| 954 | 204725_s_at | 2428.98 | 6.94 | 1.28 | 1.49 | NM_006153 | Hs.54589 | NCK1 |
| 955 | 204552_at | 2431.50 | 6.98 | 1.30 | 1.54 | AA355179 | Hs.334575 | INPP4A |
| 956 | 217671_at | 2432.92 | 7.00 | 1.28 | 1.50 | BE466926 | — | — |
| 957 | 206565_x_at | 2433.48 | 7.00 | 1.26 | 1.47 | NM_006780 | Hs.440958 | SMA3 |
| 958 | 208798_x_at | 2436.90 | 7.04 | 1.27 | 1.48 | AF204231 | Hs.182982 | GOLGIN-67 |
| 959 | 212247_at | 2436.97 | 7.04 | 1.30 | 1.53 | AW008531 | Hs.413636 | NUP205 |
| 960 | 211783_s_at | 2438.35 | 7.04 | 1.25 | 1.44 | BC006177 | Hs.101448 | MTA1 |
| 961 | 212666_at | 2439.44 | 7.06 | 1.29 | 1.52 | AB046845 | Hs.436249 | SMURF1 |
| 962 | 202499_s_at | 2441.03 | 7.08 | 1.16 | 1.28 | NM_006931 | Hs.419240 | SLC2A3 |
| 963 | 200859_x_at | 2441.44 | 7.08 | 1.27 | 1.47 | NM_001456 | Hs.195464 | FLNA |
| 964 | 209031_at | 2443.64 | 7.10 | 1.28 | 1.49 | AL519710 | Hs.156682 | IGSF4 |
| 965 | 208840_s_at | 2445.46 | 7.13 | 1.29 | 1.52 | AU149503 | Hs.303676 | G3BP2 |
| 966 | 31874_at | 2446.68 | 7.14 | 1.25 | 1.44 | Y07846 | Hs.322852 | GAS2L1 |
| 967 | 211991_s_at | 2448.13 | 7.15 | 1.22 | 1.38 | M27487 | Hs.914 | HLA-DPA1 |
| 968 | 211657_at | 2448.67 | 7.15 | 1.31 | 1.55 | M18728 | Hs.436718 | CEACAM6 |
| 969 | 219410_at | 2449.92 | 7.17 | 1.09 | 1.16 | NM_018004 | Hs.104800 | FLJ10134 |
| 970 | 208313_s_at | 2453.55 | 7.23 | 1.28 | 1.50 | NM_004630 | Hs.440835 | SF1 |
| 971 | 209636_at | 2453.75 | 7.23 | 1.28 | 1.50 | BC002844 | Hs.73090 | NFKB2 |
| 972 | 217902_s_at | 2455.01 | 7.25 | 1.29 | 1.52 | NM_004667 | Hs.434890 | HERC2 |
| 973 | 217497_at | 2455.75 | 7.25 | 1.18 | 1.30 | AW613387 | Hs.435067 | ECGF1 |
| 974 | 214575_s_at | 2456.83 | 7.26 | 1.26 | 1.46 | NM_001700 | Hs.72885 | AZU1 |
| 975 | 209004_s_at | 2459.75 | 7.29 | 1.26 | 1.46 | AF142481 | Hs.5548 | FBXL5 |
| 976 | 208450_at | 2463.01 | 7.33 | 1.23 | 1.41 | NM_006498 | Hs.113987 | LGALS2 |
| 977 | 203305_at | 2466.11 | 7.37 | 1.23 | 1.40 | NM_000129 | Hs.80424 | F13A1 |
| 978 | 213416_at | 2455.15 | 7.36 | 1.30 | 1.54 | BG532690 | — | — |
| 979 | 208690_s_at | 2468.95 | 7.40 | 1.24 | 1.43 | BC000915 | Hs.750807 | PDLIM1 |
| 980 | 209995_s_at | 2469.96 | 7.41 | 1.06 | 1.10 | BC003574 | Hs.2484 | TCL1A |
| 981 | 210663_s_at | 2470.63 | 7.42 | 1.28 | 1.50 | BC000879 | Hs.444471 | KYNU |
| 982 | 33132_at | 2475.43 | 7.47 | 1.25 | 1.45 | U37012 | Hs.83727 | CPSF1 |
| 983 | 218821_at | 2479.70 | 7.54 | 1.19 | 1.34 | AL139349 | Hs.378135 | NPEPL1 |
| 984 | 205270_s_at | 2480.61 | 7.55 | 1.29 | 1.51 | NM_005565 | Hs.2488 | LCP2 |
| 985 | 212584_at | 2481.20 | 7.55 | 1.30 | 1.53 | BG260519 | Hs.129952 | AQR |
| 986 | 205219_s_at | 2485.59 | 7.60 | 1.27 | 1.49 | NM_002044 | Hs.129228 | GALK2 |
| 987 | 202263_at | 2486.44 | 7.60 | 1.25 | 1.45 | NM_016243 | Hs.334832 | NQO3A2 |
| 988 | 201912_s_at | 2487.02 | 7.60 | 1.29 | 1.52 | NM_002094 | Hs.2707 | GSPT1 |
| 989 | 216576_x_at | 2487.22 | 7.60 | 1.19 | 1.33 | AF103529 | — | — |
| 990 | 219132_at | 2488.66 | 7.61 | 1.29 | 1.51 | NM_021255 | Hs.44038 | PELI2 |
| 991 | 208707_at | 2489.38 | 7.62 | 1.21 | 1.38 | BE552334 | Hs.433702 | EIF5 |
| 992 | 210765_at | 2489.46 | 7.61 | 1.28 | 1.50 | AF053640 | Hs.90073 | CSE1L |
| 993 | 201890_at | 2490.35 | 7.62 | 1.30 | 1.54 | BE966236 | Hs.226390 | RRM2 |
| 994 | 220088_at | 2491.49 | 7.63 | 1.23 | 1.41 | NM_001736 | — | C5R1 |
| 995 | 201772_at | 2491.77 | 7.63 | 1.26 | 1.47 | NM_015878 | Hs.223014 | OAZIN |
| 996 | 201197_at | 2497.28 | 7.71 | 1.22 | 1.39 | NM_001634 | Hs.159118 | AMD1 |
| 997 | 212057_at | 2499.81 | 7.74 | 1.28 | 1.51 | AA206161 | Hs.222171 | KIAA0182 |
| 998 | 217963_s_at | 2501.64 | 7.75 | 1.20 | 1.34 | NM_014380 | Hs.381039 | NGFRAP1 |
| 999 | 221485_at | 2501.75 | 7.74 | 1.21 | 1.36 | AL035683 | Hs.107526 | B4GALT5 |
| 1000 | 60084_at | 2501.79 | 7.73 | 1.29 | 1.51 | AI453099 | Hs.386952 | CYLD |
| 1001 | 209835_x_at | 2503.29 | 7.76 | 1.28 | 1.50 | BC004372 | Hs.306278 | CD44 |
| 1002 | 220924_s_at | 2503.54 | 7.75 | 1.28 | 1.51 | NM_018976 | Hs.298275 | SLC38A2 |
| 1003 | 222023_at | 2505.00 | 7.76 | 1.26 | 1.46 | AK022014 | Hs.350631 | AKAP13 |
| 1004 | 201619_at | 2505.10 | 7.75 | 1.26 | 1.46 | NM_006793 | Hs.397062 | PRDX3 |
| 1005 | 201823_s_at | 2507.91 | 7.78 | 1.26 | 1.45 | NM_004290 | Hs.170926 | RNF14 |
| 1006 | 212612_at | 2512.26 | 7.86 | 1.29 | 1.53 | D31888 | Hs.405373 | RCOR1 |
| 1007 | 214430_at | 2513.79 | 7.87 | 1.25 | 1.44 | NM_000169 | Hs.69089 | GLA |
| 1008 | 215118_s_at | 2516.64 | 7.91 | 1.22 | 1.39 | AW519168 | Hs.366 | MGC27165 |
| 1009 | 200965_s_at | 2517.87 | 7.92 | 1.27 | 1.48 | NM_006720 | Hs.442540 | ABLIM1 |
| 1010 | 201239_s_at | 2518.76 | 7.92 | 1.23 | 1.41 | BF530535 | Hs.87095 | KIAA0102 |
| 1011 | 212602_at | 2518.97 | 7.92 | 1.26 | 1.46 | AI806395 | Hs.105340 | WDFY3 |
| 1012 | 208938_at | 2519.55 | 7.92 | 1.28 | 1.51 | BC004913 | Hs.9629 | PRCC |
| 1013 | 220296_at | 2525.00 | 7.99 | 1.27 | 1.48 | NM_024564 | Hs.13785 | GALNT10 |
| 1014 | 203907_s_at | 2526.49 | 8.01 | 1.26 | 1.47 | NM_014869 | Hs.244616 | IQSEC1 |
| 1015 | 202941_at | 2529.24 | 8.03 | 1.28 | 1.49 | NM_021074 | Hs.51299 | NDUFV2 |
| 1016 | 213753_x_at | 2530.08 | 8.04 | 1.23 | 1.40 | BF541557 | — | — |
| 1017 | 215203_at | 2530.15 | 8.03 | 1.29 | 1.53 | AW438464 | Hs.434433 | GOLGA4 |
| 1018 | 212441_at | 2530.25 | 8.03 | 1.27 | 1.48 | D86985 | Hs.79276 | KIAA0232 |
| 1019 | 217047_s_at | 2531.96 | 8.05 | 1.28 | 1.50 | AK027138 | Hs.442818 | FAM13A1 |
| 1020 | 219998_at | 2533.52 | 8.06 | 1.17 | 1.29 | NM_014181 | Hs.372208 | HSPC159 |
| 1021 | 210759_s_at | 2535.64 | 8.09 | 1.27 | 1.49 | M64992 | Hs.82159 | PSMA1 |
| 1022 | 204618_s_at | 2536.26 | 8.10 | 1.28 | 1.51 | NM_005254 | Hs.181202 | GABPB2 |
| 1023 | 205267_at | 2536.42 | 8.09 | 1.25 | 1.45 | NM_006235 | Hs.2407 | POU2AF1 |
| 1024 | 213538_at | 2537.88 | 8.11 | 1.27 | 1.47 | AI936458 | Hs.430541 | SON |
| 1025 | 209185_s_at | 2539.69 | 8.13 | 1.27 | 1.47 | AF073310 | Hs.143648 | IRS2 |
| 1026 | 209941_at | 2540.06 | 8.13 | 1.29 | 1.51 | U50062 | Hs.390758 | RIPK1 |
| 1027 | 219452_at | 2540.90 | 8.14 | 1.27 | 1.47 | NM_022365 | Hs.499331 | DPEP2 |
| 1028 | 203620_s_at | 2541.83 | 8.14 | 1.27 | 1.48 | NM_014824 | Hs.19056 | FCHSD2 |
| 1029 | 208890_s_at | 2543.20 | 8.16 | 1.24 | 1.43 | BC004542 | Hs.3989 | PLXNB2 |
| 1030 | 202161_at | 2543.77 | 8.17 | 1.25 | 1.45 | NM_002741 | Hs.2499 | PKN1 |
| 1031 | 201435_s_at | 2544.19 | 8.17 | 1.27 | 1.48 | AW268640 | Hs.79306 | EIF4E |
| 1032 | 217526_at | 2547.25 | 8.20 | 1.28 | 1.50 | AI478300 | Hs.334788 | FLJ14639 |
| 1033 | 201302_at | 2547.38 | 8.20 | 1.26 | 1.45 | NM_001153 | Hs.422986 | ANXA4 |
| 1034 | 201490_s_at | 2547.74 | 8.19 | 1.25 | 1.43 | NM_005729 | Hs.381072 | PPIF |
| 1035 | 208319_s_at | 2548.16 | 8.19 | 1.28 | 1.50 | NM_006743 | Hs.301404 | RBM3 |
| 1036 | 212461_at | 2548.35 | 8.19 | 1.26 | 1.46 | BF793951 | — | — |
| 1037 | 212730_at | 2549.30 | 8.20 | 1.24 | 1.43 | AK026420 | Hs.381347 | DMN |
| 1038 | 213598_at | 2555.24 | 8.28 | 1.24 | 1.43 | W87688 | — | — |
| 1039 | 218456_at | 2555.91 | 8.29 | 1.27 | 1.48 | NM_023925 | Hs.234355 | C1QDC1 |
| 1040 | 200806_s_at | 2556.49 | 8.30 | 1.21 | 1.36 | BE256479 | Hs.79037 | HSPD1 |
| 1041 | 206115_at | 2556.50 | 8.29 | 1.24 | 1.41 | NM_004430 | — | EGR3 |
| 1042 | 202251_at | 2558.12 | 8.31 | 1.27 | 1.48 | NM_004698 | Hs.11776 | PRPF3 |
| 1043 | 203521_s_at | 2558.44 | 8.31 | 1.28 | 1.50 | NM_014345 | Hs.147868 | ZNF318 |
| 1044 | 221060_s_at | 2558.57 | 8.31 | 1.27 | 1.49 | NM_003266 | Hs.174312 | TLR4 |
| 1045 | 202078_at | 2559.57 | 8.32 | 1.29 | 1.51 | NM_003653 | Hs.6076 | COPS3 |
| 1046 | 202731_at | 2561.59 | 8.34 | 1.27 | 1.48 | NM_014456 | Hs.257697 | PDCD4 |
| 1047 | 215936_s_at | 2562.70 | 8.35 | 1.22 | 1.39 | AK001657 | Hs.12144 | KIAA1033 |
| 1048 | 202446_s_at | 2562.80 | 8.34 | 1.23 | 1.41 | AI825926 | Hs.348478 | PLSCR1 |
| 1049 | 218008_at | 2565.46 | 8.37 | 1.27 | 1.49 | NM_017994 | Hs.287955 | FLJ10099 |
| 1050 | 210978_s_at | 2566.50 | 8.38 | 1.28 | 1.49 | BC002616 | Hs.406504 | TAGLN2 |
| 1051 | 214196_s_at | 2566.88 | 8.37 | 1.26 | 1.46 | AA602532 | Hs.429658 | CLN2 |
| 1052 | 219016_at | 2566.98 | 8.37 | 1.28 | 1.49 | NM_021826 | — | FLJ13149 |
| 1053 | 213383_at | 2568.61 | 8.38 | 1.19 | 1.33 | AW593269 | — | — |
| 1054 | 205173_x_at | 2568.68 | 8.37 | 1.23 | 1.40 | NM_001779 | Hs.75626 | CD58 |
| 1055 | 210479_s_at | 2571.32 | 8.42 | 1.15 | 1.26 | L14611 | Hs.528666 | RORA |
| 1056 | 212608_s_at | 2572.29 | 8.43 | 1.27 | 1.48 | W85912 | Hs.188882 | — |
| 1057 | 217877_s_at | 2573.92 | 8.45 | 1.26 | 1.47 | NM_021639 | Hs.169854 | SP192 |
| 1058 | 207419_s_at | 2574.44 | 8.45 | 1.21 | 1.37 | NM_002872 | Hs.301175 | RAC2 |
| 1059 | 201310_s_at | 2575.21 | 8.45 | 1.24 | 1.43 | NM_004772 | Hs.508741 | C5orf13 |
| 1060 | 206100_at | 2575.95 | 8.46 | 1.28 | 1.49 | NM_001874 | Hs.334873 | CPM |
| 1061 | 221755_at | 2577.93 | 8.49 | 1.22 | 1.39 | BG334196 | Hs.438942 | DKFZp762C186 |
| 1062 | 216929_x_at | 2580.57 | 8.53 | 1.25 | 1.44 | U15197 | Hs.113271 | ABO |
| 1063 | 215023_s_at | 2580.77 | 8.53 | 1.27 | 1.48 | AC000064 | Hs.164682 | PEX1 |
| 1064 | 207697_x_at | 2581.22 | 8.52 | 1.23 | 1.40 | NM_005874 | Hs.306230 | LILRB2 |
| 1065 | 202912_at | 2582.55 | 8.54 | 1.07 | 1.12 | NM_001124 | Hs.441047 | ADM |
| 1066 | 210732_s_at | 2583.64 | 8.55 | 1.26 | 1.45 | AF342816 | Hs.4082 | LGALS8 |
| 1067 | 214916_x_at | 2583.74 | 8.54 | 1.27 | 1.47 | BG340548 | Hs.366 | MGC27165 /// |
| | | | | | | | | IGHG1 |
| 1068 | 208852_s_at | 2583.84 | 8.53 | 1.23 | 1.40 | AI761759 | Hs.155560 | CANX |
| 1069 | 218402_s_at | 2583.96 | 8.53 | 1.29 | 1.51 | NM_022081 | Hs.441481 | HPS4 |
| 1070 | 209447_at | 2585.72 | 8.55 | 1.27 | 1.48 | AF043290 | Hs.282117 | SYNE1 |
| 1071 | 211984_at | 2588.84 | 8.60 | 1.28 | 1.49 | AI653730 | Hs.282410 | CALM1 |
| 1072 | 211272_s_at | 2588.90 | 8.59 | 1.26 | 1.46 | AF064771 | Hs.172690 | DGKA |
| 1073 | 203856_at | 2589.18 | 8.59 | 1.27 | 1.47 | NM_003384 | Hs.422662 | VRK1 |
| 1074 | 218123_at | 2590.94 | 8.61 | 1.28 | 1.49 | NM_017835 | Hs.5811 | C21orf59 |
| 1075 | 208954_s_at | 2591.87 | 8.62 | 1.28 | 1.51 | BC003381 | Hs.192881 | KIAA0217 |
| 1076 | 202121_s_at | 2593.63 | 8.64 | 1.23 | 1.41 | NM_014453 | Hs.12107 | BC-2 |
| 1077 | 32099_at | 2594.98 | 8.67 | 1.28 | 1.50 | D50928 | Hs.159384 | SAFB2 |
| 1078 | 219797_at | 2595.36 | 8.67 | 1.25 | 1.45 | NM_012214 | Hs.177576 | MGAT4A |
| 1079 | 210415_s_at | 2595.78 | 8.67 | 1.28 | 1.50 | AF053970 | Hs.129055 | ODF2 |
| 1080 | 210180_s_at | 2596.16 | 8.67 | 0.98 | 0.97 | U87836 | Hs.30035 | SFRS10 |
| 1081 | 220952_s_at | 2596.46 | 8.67 | 1.24 | 1.43 | NM_019012 | Hs.242537 | PLEKHA5 |
| 1082 | 212387_at | 2598.54 | 8.70 | 1.21 | 1.37 | BG495771 | Hs.359289 | TCF4 |
| 1083 | 212330_at | 2600.82 | 8.74 | 1.29 | 1.51 | R60866 | Hs.79353 | TFDP1 |
| 1084 | 213596_at | 2601.49 | 8.74 | 1.19 | 1.34 | AL050391 | Hs.74122 | CASP4 |
| 1085 | 200707_at | 2601.86 | 8.74 | 1.29 | 1.51 | NM_002743 | Hs.512640 | PRKCSH |
| 1086 | 210950_s_at | 2603.40 | 8.76 | 1.19 | 1.34 | BC003573 | Hs.191435 | FDFT1 |
| 1087 | 201643_x_at | 2603.70 | 8.76 | 1.28 | 1.50 | NM_016604 | Hs.24125 | JMJD1B |
| 1088 | 204049_s_at | 2604.75 | 8.77 | 1.28 | 1.50 | NM_014721 | Hs.102471 | PHACTR2 |
| 1089 | 213761_at | 2605.76 | 8.77 | 1.26 | 1.46 | AW664850 | Hs.136623 | MDM1 |
| 1090 | 205495_s_at | 2607.88 | 8.80 | 1.02 | 1.04 | NM_006433 | Hs.105806 | GNLY |
| 1091 | 212894_at | 2608.01 | 8.79 | 1.28 | 1.50 | NM_003171 | Hs.106469 | SUPV3L1 |
| 1092 | 201438_at | 2609.56 | 8.81 | 1.23 | 1.40 | NM_004369 | Hs.233240 | COL6A3 |
| 1093 | 203016_s_at | 2610.88 | 8.83 | 1.18 | 1.31 | AK001710 | Hs.22587 | SSX2IP |
| 1094 | 219078_at | 2611.23 | 8.83 | 1.27 | 1.48 | NM_018040 | Hs.53913 | GPATC2 |
| 1095 | 211101_x_at | 2615.90 | 8.90 | 1.24 | 1.42 | U82276 | — | LILRA2 |
| 1096 | 212830_at | 2620.39 | 8.98 | 1.27 | 1.47 | W68084 | Hs.236216 | EGFL5 |
| 1097 | 204192_at | 2622.18 | 9.02 | 1.21 | 1.38 | NM_001774 | Hs.166556 | CD37 |
| 1098 | 200985_s_at | 2622.58 | 9.01 | 1.23 | 1.41 | NM_000611 | Hs.278573 | CD59 |
| 1099 | 213700_s_at | 2623.73 | 9.02 | 1.25 | 1.43 | AA554945 | — | — |
| 1100 | 212403_at | 2623.76 | 9.01 | 1.28 | 1.49 | AI749193 | Hs.305834 | UBE3B |
| 1101 | 215640_at | 2627.25 | 9.07 | 1.26 | 1.45 | AK000173 | Hs.438702 | KIAA1055 |
| 1102 | 219189_at | 2627.67 | 9.07 | 1.27 | 1.48 | NM_024555 | Hs.12271 | FBXL6 |
| 1103 | 202123_s_at | 2631.70 | 9.14 | 1.27 | 1.48 | NM_005157 | Hs.446504 | ABL1 |
| 1104 | 215735_s_at | 2632.13 | 9.14 | 1.27 | 1.47 | AC005600 | Hs.90303 | TSC2 |
| 1105 | 200629_at | 2633.04 | 9.14 | 1.12 | 1.21 | NM_004184 | Hs.82030 | WARS |
| 1106 | 202216_x_at | 2633.71 | 9.15 | 1.28 | 1.50 | BC005003 | Hs.285133 | NFYC |
| 1107 | 212374_at | 2639.48 | 9.23 | 1.21 | 1.36 | NM_015322 | Hs.362733 | FEM1B |
| 1108 | 211559_s_at | 2640.62 | 9.25 | 1.26 | 1.47 | L49506 | Hs.13291 | CCNG2 |
| 1109 | 217889_s_at | 2641.88 | 9.26 | 1.27 | 1.48 | NM_024843 | Hs.31297 | CYBRD1 |
| 1110 | 208991_at | 2642.44 | 9.26 | 1.25 | 1.44 | AA634272 | Hs.421342 | STAT3 |
| 1111 | 208946_s_at | 2642.45 | 9.25 | 1.28 | 1.49 | AF139131 | Hs.12272 | BECN1 |
| 1112 | 201418_s_at | 2645.18 | 9.30 | 1.19 | 1.33 | NM_003107 | Hs.357901 | SOX4 |
| 1113 | 201178_at | 2646.10 | 9.31 | 1.26 | 1.46 | NM_012179 | Hs.5912 | FBXO7 |
| 1114 | 206398_s_at | 2646.21 | 9.31 | 1.26 | 1.46 | NM_001770 | — | — |
| 1115 | 216401_x_at | 2646.71 | 9.31 | 1.24 | 1.43 | AJ408433 | — | — |
| 1116 | 211996_s_at | 2647.34 | 9.31 | 1.19 | 1.33 | BG256504 | Hs.512019 | KIAA0220 |
| 1117 | 202469_s_at | 2648.15 | 9.31 | 1.27 | 1.48 | AU149367 | Hs.64542 | CPSF6 |
| 1118 | 218509_at | 2649.87 | 9.34 | 1.26 | 1.45 | NM_022737 | Hs.6846 | LPPR2 |
| 1119 | 215548_s_at | 2653.22 | 9.39 | 1.28 | 1.50 | AB020724 | Hs.27023 | SCFD1 |
| 1120 | 210746_s_at | 2653.86 | 9.40 | 1.25 | 1.45 | M30646 | Hs.368642 | EPB42 |
| 1121 | 205119_s_at | 2654.35 | 9.40 | 1.15 | 1.25 | NM_002029 | — | — |
| 1122 | 219157_at | 2657.09 | 9.45 | 1.26 | 1.47 | NM_007246 | Hs.122967 | KLHL2 |
| 1123 | 203097_s_at | 2657.26 | 9.44 | 1.27 | 1.49 | NM_014247 | Hs.373588 | RAPGEF2 |
| 1124 | 208546_x_at | 2657.86 | 9.45 | 1.05 | 1.08 | NM_003524 | — | — |
| 1125 | 211734_s_at | 2659.84 | 9.47 | 1.16 | 1.28 | BC005912 | Hs.897 | FCER1A |
| 1126 | 218345_at | 2660.51 | 9.48 | 0.92 | 0.87 | NM_018487 | Hs.12126 | HCA112 |
| 1127 | 213353_at | 2661.62 | 9.49 | 1.24 | 1.43 | BF693921 | Hs.421474 | ABCA5 |
| 1128 | 202032_s_at | 2661.75 | 9.48 | 1.26 | 1.45 | NM_006122 | Hs.116459 | MAN2A2 |
| 1129 | 212975_at | 2664.54 | 9.53 | 1.23 | 1.41 | AB020677 | Hs.18166 | KIAA0870 |
| 1130 | 205099_s_at | 2669.68 | 9.59 | 1.23 | 1.40 | NM_001295 | Hs.301921 | CCR1 |
| 1131 | 204799_at | 2669.85 | 9.59 | 1.27 | 1.49 | NM_014838 | Hs.13604 | ZBED4 |
| 1132 | 212311_at | 2671.44 | 9.61 | 1.27 | 1.48 | AA522514 | Hs.49500 | KIAA0746 |
| 1133 | 201970_s_at | 2673.14 | 9.63 | 1.27 | 1.48 | NM_002482 | Hs.446206 | NASP |
| 1134 | 200791_s_at | 2674.29 | 9.64 | 1.26 | 1.45 | NM_003870 | Hs.1742 | IQGAP1 |
| 1135 | 207856_s_at | 2676.03 | 9.67 | 1.26 | 1.47 | NM_017951 | Hs.94491 | FLJ41352 /// |
| | | | | | | | | FLJ20297 |
| 1136 | 210649_s_at | 2676.95 | 9.68 | 1.27 | 1.49 | AF231056 | Hs.170333 | ARID1A |
| 1137 | 204108_at | 2677.92 | 9.68 | 1.27 | 1.47 | AL031778 | Hs.10441 | NFYA |
| 1138 | 201908_at | 2678.04 | 9.67 | 1.27 | 1.48 | NM_004423 | Hs.381928 | DVL3 |
| 1139 | 201087_at | 2678.69 | 9.68 | 1.24 | 1.43 | NM_002859 | Hs.446336 | PXN |
| 1140 | 206752_s_at | 2679.12 | 9.68 | 1.24 | 1.42 | NM_004402 | Hs.133089 | DFFB |
| 1141 | 201881_s_at | 2679.34 | 9.68 | 1.27 | 1.48 | NM_005744 | Hs.181461 | ARIH1 |
| 1142 | 220576_at | 2679.92 | 9.68 | 1.25 | 1.44 | NM_024989 | Hs.528683 | PGAP1 |
| 1143 | 209845_at | 2680.46 | 9.68 | 1.26 | 1.47 | AF117233 | Hs.7838 | MKRN1 |
| 1144 | 221205_at | 2684.37 | 9.76 | 1.23 | 1.41 | NM_018041 | — | — |
| 1145 | 213624_at | 2685.33 | 9.77 | 1.27 | 1.49 | AA873600 | Hs.277962 | SMPDL3A |
| 1146 | 211536_x_at | 2685.52 | 9.76 | 1.28 | 1.51 | AB009358 | Hs.290346 | MAP3K7 |
| 1147 | 208927_at | 2687.32 | 9.79 | 1.25 | 1.45 | BF673888 | Hs.129951 | SPOP |
| 1148 | 203431_s_at | 2689.71 | 9.82 | 1.24 | 1.43 | NM_014715 | Hs.440379 | RICS |
| 1149 | 211911_x_at | 2692.79 | 9.87 | 1.24 | 1.43 | L07950 | Hs.274485 | HLA-C |
| 1150 | 201096_s_at | 2696.20 | 9.92 | 1.27 | 1.47 | AL537042 | Hs.435639 | ARF4 |
| 1151 | 221477_s_at | 2698.20 | 9.95 | 1.24 | 1.43 | BF575213 | Hs.384944 | SOD2 |
| 1152 | 217750_s_at | 2699.56 | 9.96 | 1.24 | 1.43 | NM_023079 | Hs.369120 | FLJ13855 |
| 1153 | 217741_s_at | 2700.46 | 9.96 | 1.11 | 1.18 | AW471220 | Hs.406096 | ZA20D2 |
| 1154 | 217853_at | 2704.06 | 10.02 | 1.25 | 1.44 | NM_022748 | Hs.12210 | TENS1 |
| 1155 | 213605_s_at | 2704.15 | 10.01 | 1.26 | 1.46 | AL049987 | Hs.482464 | — |
| 1156 | 209020_at | 2704.56 | 10.01 | 1.26 | 1.46 | AF217514 | Hs.75798 | C20orf111 |
| 1157 | 204588_s_at | 2705.82 | 10.02 | 1.24 | 1.42 | NM_003982 | Hs.194693 | SLC7A7 |
| 1158 | 208846_s_at | 2706.02 | 10.01 | 1.19 | 1.32 | U90943 | Hs.439253 | VDAC3 |
| 1159 | 203385_at | 2706.45 | 10.01 | 1.25 | 1.45 | NM_001345 | Hs.172690 | DGKA |
| 1160 | 204094_s_at | 2706.82 | 10.01 | 1.29 | 1.51 | NM_014779 | Hs.52526 | KIAA0669 |
| 1161 | 201369_s_at | 2708.89 | 10.05 | 1.18 | 1.31 | NM_006887 | Hs.78909 | ZFP36L2 |
| 1162 | 213471_at | 2708.96 | 10.04 | 1.25 | 1.44 | AB014573 | Hs.434931 | NPHP4 |
| 1163 | 212246_at | 2710.85 | 10.07 | 1.25 | 1.44 | BE880828 | Hs.293689 | MCFD2 |
| 1164 | 208130_s_at | 2712.17 | 10.09 | 1.22 | 1.39 | NM_030984 | Hs.444510 | TBXAS1 |
| 1165 | 202089_s_at | 2712.89 | 10.10 | 1.20 | 1.35 | NM_012319 | Hs.79136 | SLC39A6 |
| 1166 | 215209_at | 2713.90 | 10.11 | 1.26 | 1.46 | AU143984 | Hs.409424 | SEC24D |
| 1167 | 212871_at | 2714.14 | 10.11 | 1.27 | 1.49 | NM_003668 | Hs.413901 | MAPKAPK5 |
| 1168 | 213521_at | 2717.10 | 10.16 | 1.24 | 1.41 | AW575379 | — | — |
| 1169 | 203355_s_at | 2718.51 | 10.18 | 1.26 | 1.46 | NM_015310 | Hs.236438 | PSD3 |
| 1170 | 203909_at | 2720.31 | 10.20 | 1.28 | 1.51 | NM_006359 | Hs.62185 | SLC9A6 |
| 1171 | 220397_at | 2722.04 | 10.23 | 1.24 | 1.42 | NM_020128 | Hs.136623 | MDM1 |
| 1172 | 222175_s_at | 2722.22 | 10.22 | 1.25 | 1.44 | AK000003 | Hs.410347 | PCQAP |
| 1173 | 203159_at | 2723.55 | 10.24 | 1.27 | 1.48 | NM_014905 | Hs.128410 | GLS |
| 1174 | 20777_s_at | 2725.07 | 10.26 | 1.26 | 1.47 | NM_007237 | Hs.399826 | SP140 |
| 1175 | 219833_s_at | 2727.23 | 10.30 | 1.23 | 1.41 | NM_018100 | Hs.446047 | EFHC1 |
| 1176 | 39402_at | 2729.05 | 10.32 | 1.23 | 1.40 | M15330 | Hs.126256 | IL1B |
| 1177 | 200828_s_at | 2729.79 | 10.33 | 1.23 | 1.41 | BE871379 | Hs.97128 | ZNF207 |
| 1178 | 218109_s_at | 2729.88 | 10.33 | 1.24 | 1.41 | NM_022736 | Hs.7503 | FLJ14153 |
| 1179 | 204958_at | 2730.15 | 10.32 | 1.26 | 1.46 | NM_004073 | Hs.153640 | PLK3 |
| 1180 | 205067_at | 2730.17 | 10.31 | 1.23 | 1.41 | NM_000576 | Hs.126256 | IL1B |
| 1181 | 210113_s_at | 2730.73 | 10.31 | 1.23 | 1.41 | AF310105 | Hs.104305 | NALP1 |
| 1182 | 202215_s_at | 2731.06 | 10.31 | 1.26 | 1.47 | NM_014223 | Hs.285133 | NFYC |
| 1183 | 209379_s_at | 2731.22 | 10.30 | 1.24 | 1.42 | AF241785 | Hs.81897 | KIAA1128 |
| 1184 | 202457_s_at | 2731.93 | 10.31 | 1.26 | 1.47 | AA911231 | Hs.272458 | PPP3CA |
| 1185 | 219563_at | 2732.60 | 10.31 | 1.27 | 1.48 | NM_024633 | Hs.41502 | C14orf139 |
| 1186 | 206390_x_at | 2732.69 | 10.31 | 1.20 | 1.35 | NM_002619 | Hs.81564 | PF4 |
| 1187 | 211302_s_at | 2733.36 | 10.31 | 1.28 | 1.49 | L20966 | Hs.188 | PDE4B |
| 1188 | 201237_at | 2735.25 | 10.35 | 1.25 | 1.44 | AV685920 | Hs.369579 | CAPZA2 |
| 1189 | 219994_at | 2737.59 | 10.38 | 1.23 | 1.40 | NM_019043 | Hs.397115 | APBB1IP |
| 1190 | 216206_x_at | 2738.33 | 10.38 | 1.24 | 1.41 | BC005365 | Hs.110299 | MAP2K7 |
| 1191 | 212219_at | 2742.43 | 10.45 | 1.26 | 1.46 | D38521 | Hs.119563 | PSME4 |
| 1192 | 210517_s_at | 2742.78 | 10.45 | 1.26 | 1.46 | AB003476 | Hs.197081 | AKAP12 |
| 1193 | 221841_s_at | 2743.05 | 10.44 | 1.23 | 1.40 | BF514079 | Hs.376206 | KLF4 |
| 1194 | 214433_s_at | 2743.34 | 10.44 | 1.24 | 1.42 | NM_003944 | Hs.334841 | SELENBP1 |
| 1195 | 212687_at | 2743.93 | 10.44 | 1.22 | 1.38 | AL110164 | Hs.112378 | LIMS1 |
| 1196 | 210951_x_at | 2744.21 | 10.44 | 124 | 1.42 | AF125393 | Hs.298530 | RAB27A |
| 1197 | 212390_at | 2746.22 | 10.47 | 1.25 | 1.45 | AB007923 | Hs.502577 | PDE4DIP |
| 1198 | 209131_s_at | 2746.32 | 10.47 | 1.23 | 1.40 | U55936 | Hs.202308 | SNAP23 |
| 1199 | 210426_x_at | 2750.88 | 10.54 | 1.14 | 1.25 | U04897 | Hs.528666 | RORA |
| 1200 | 200664_s_at | 2752.75 | 10.57 | 1.21 | 1.37 | BG537255 | Hs.82646 | DNAJB1 |
| 1201 | 201688_s_at | 2752.78 | 10.56 | 1.24 | 1.43 | BG389015 | Hs.162089 | TPD52 |
| 1202 | 208018_s_at | 2755.04 | 10.60 | 1.23 | 1.40 | NM_002110 | Hs.89555 | HCK |
| 1203 | 212698_s_at | 2758.24 | 10.66 | 1.27 | 1.47 | BF966021 | Hs.355455 | SEPT10 |
| 1204 | 212312_at | 2758.94 | 10.66 | 1.22 | 1.39 | AL117381 | Hs.305890 | BCL2L1 |
| 1205 | 202014_at | 2759.21 | 10.66 | 1.11 | 1.18 | NM_014330 | Hs.76556 | PPP1R15A |
| 1206 | 213690_s_at | 2760.08 | 10.67 | 1.25 | 1.44 | AL050131 | Hs.227429 | PTOV1 |
| 1207 | 221958_s_at | 2760.77 | 10.68 | 1.27 | 1.47 | AA775681 | Hs.297792 | FLJ23091 |
| 1208 | 202181_at | 2761.66 | 10.68 | 1.25 | 1.44 | NM_014734 | Hs.82426 | KIAA0247 |
| 1209 | 201238_s_at | 2765.36 | 10.74 | 1.24 | 1.43 | BC005338 | Hs.369579 | CAPZA2 |
| 1210 | 212307_s_at | 2766.09 | 10.75 | 1.24 | 1.42 | BF001665 | Hs.405410 | OGT |
| 1211 | 209333_at | 2766.62 | 10.75 | 1.26 | 1.46 | AB018265 | Hs.47061 | ULK1 |
| 1212 | 208643_s_at | 2767.72 | 10.76 | 1.22 | 1.38 | J04977 | Hs.257082 | XRCC5 |
| 1213 | 203752_s_at | 2768.34 | 10.76 | 1.15 | 1.27 | NM_005354 | Hs.2780 | JUND |
| 1214 | 202459_s_at | 2769.81 | 10.79 | 1.25 | 1.44 | U55968 | Hs.437425 | LPIN2 |
| 1215 | 207563_s_at | 2770.30 | 10.79 | 1.25 | 1.44 | U77413 | Hs.405410 | OGT |
| 1216 | 213182_x_at | 2770.78 | 10.78 | 1.18 | 1.32 | R78668 | Hs.106070 | CDKN1C |
| 1217 | 210276_s_at | 2772.67 | 10.82 | 1.26 | 1.46 | AF281030 | Hs.40342 | HRIHFB2122 |
| 1218 | 206877_at | 2778.04 | 10.91 | 1.24 | 1.41 | NM_002357 | Hs.379930 | MAD |
| 1219 | 208704_x_at | 2778.57 | 10.91 | 1.26 | 1.46 | BC000373 | Hs.279518 | APLP2 |
| 1220 | 218229_s_at | 2781.32 | 10.95 | 1.25 | 1.44 | NM_017542 | Hs.432752 | POGK |
| 1221 | 208781_x_at | 2781.40 | 10.94 | 1.23 | 1.41 | AF062483 | Hs.12102 | SNX3 |
| 1222 | 218107_at | 2782.49 | 10.95 | 1.27 | 1.47 | NM_025160 | Hs.406339 | WDR26 |
| 1223 | 221986_s_at | 2782.54 | 10.94 | 1.23 | 1.40 | AW006750 | Hs.246875 | DRE1 |
| 1224 | 221645_s_at | 2784.09 | 10.96 | 1.24 | 1.43 | M27877 | Hs.305953 | ZNF83 |
| 1225 | 212130_x_at | 2785.84 | 10.98 | 1.24 | 1.42 | AL537707 | Hs.150580 | SUI1 |
| 1226 | 205054_at | 2786.98 | 11.00 | 1.26 | 1.47 | NM_004543 | Hs.386131 | NEB |
| 1227 | 220746_s_at | 2788.25 | 11.03 | 1.26 | 1.46 | NM_016290 | Hs.300263 | RAP80 |
| 1228 | 208868_s_at | 2790.36 | 11.06 | 1.23 | 1.41 | BF125756 | Hs.336429 | GABARAPL1 |
| 1229 | 201748_s_at | 2790.96 | 11.07 | 1.26 | 1.46 | NM_002967 | Hs.23978 | SAFB |
| 1230 | 209117_at | 2791.81 | 11.08 | 1.18 | 1.32 | U79458 | Hs.231840 | WBP2 |
| 1231 | 216232_s_at | 2792.78 | 11.08 | 1.26 | 1.46 | AI697055 | Hs.298716 | GCN1L1 |
| 1232 | 218908_at | 2793.75 | 11.09 | 1.25 | 1.45 | NM_024083 | Hs.298351 | ASPSCR1 |
| 1233 | 212308_at | 2794.84 | 11.11 | 1.27 | 1.48 | AL137636 | Hs.108614 | CLASP2 |
| 1234 | 209234_at | 2795.36 | 11.12 | 1.26 | 1.45 | BF939474 | Hs.444757 | KIF1B |
| 1235 | 205842_s_at | 2796.33 | 11.13 | 1.25 | 1.44 | AF001362 | Hs.434374 | JAK2 |
| 1236 | 206099_at | 2796.73 | 11.13 | 1.24 | 1.43 | NM_006255 | Hs.315366 | PRKCH |
| 1237 | 201012_at | 2797.24 | 11.13 | 1.26 | 1.47 | NM_000700 | Hs.287558 | ANXA1 |
| 1238 | 210588_x_at | 2798.45 | 11.14 | 1.26 | 1.46 | L32610 | Hs.156481 | HNRPH3 |
| 1239 | 211537_x_at | 2799.23 | 11.14 | 1.26 | 1.46 | AF218074 | Hs.290346 | MAP3K7 |
| 1240 | 201195_s_at | 2799.99 | 11.15 | 1.25 | 1.43 | AB018009 | Hs.184601 | SLC7A5 |
| 1241 | 203319_s_at | 2800.12 | 11.14 | 1.26 | 1.46 | L04282 | Hs.442787 | ZNF148 |
| 1242 | 211672_s_at | 2800.74 | 11.14 | 1.23 | 1.41 | AF019888 | Hs.323342 | ARPC4 |
| 1243 | 219679_s_at | 2802.25 | 11.17 | 1.18 | 1.31 | NM_018604 | Hs.370152 | WAC |
| 1244 | 218967_s_at | 2802.35 | 11.16 | 1.26 | 1.47 | NM_030664 | Hs.397381 | PTER |
| 1245 | 218360_at | 2805.58 | 11.22 | 1.27 | 1.47 | NM_020673 | Hs.281117 | RAB22A |
| 1246 | 212060_at | 2806.41 | 11.23 | 1.25 | 1.44 | AU152088 | Hs.370907 | SR140 |
| 1247 | 203096_s_at | 2808.17 | 11.26 | 1.07 | 1.12 | BF439282 | Hs.373588 | RAPGEF2 |
| 1248 | 215684_s_at | 2808.19 | 11.25 | 1.27 | 1.48 | AL096741 | Hs.436407 | ASCC2 |
| 1249 | 210788_s_at | 2809.09 | 11.26 | 1.17 | 1.29 | AF126782 | Hs.36049 | DHRS7 |
| 1250 | 213376_at | 2809.24 | 11.26 | 1.26 | 1.47 | AI656706 | Hs.511938 | ZBTB1 |
| 1251 | 203821_at | 2809.92 | 11.26 | 1.21 | 1.37 | NM_001945 | Hs.799 | DTR |
| 1252 | 204048_s_at | 2810.07 | 11.26 | 1.26 | 1.46 | AA551142 | Hs.102471 | PHACTR2 |
| 1253 | 202080_s_at | 2811.05 | 11.27 | 1.27 | 1.49 | NM_014965 | Hs.457063 | OIP106 |
| 1254 | 219565_at | 2812.51 | 11.29 | 1.27 | 1.47 | NM_020674 | Hs.446065 | CYP20A1 |
| 1255 | 200634_at | 2813.53 | 11.30 | 1.24 | 1.43 | NM_005022 | Hs.408943 | PFN1 |
| 1256 | 201866_s_at | 2813.56 | 11.29 | 1.25 | 1.44 | NM_000176 | — | — |
| 1257 | 202599_s_at | 2814.33 | 11.30 | 1.28 | 1.49 | NM_003489 | Hs.155017 | NRIP1 |
| 1258 | 210859_x_at | 2814.40 | 11.29 | 1.25 | 1.44 | AF077973 | Hs.194660 | CLN3 |
| 1259 | 212756_s_at | 2815.06 | 11.29 | 1.24 | 1.43 | AI761518 | Hs.131842 | C6orf133 |
| 1260 | 219534_x_at | 2816.17 | 11.30 | 1.16 | 1.28 | NM_000076 | Hs.106070 | CDKN1C |
| 1261 | 201715_s_at | 2816.65 | 11.30 | 1.26 | 1.45 | NM_014977 | Hs.227133 | ACIN1 |
| 1262 | 207492_at | 2821.11 | 11.40 | 1.24 | 1.42 | NM_025105 | Hs.63657 | NGLY1 |
| 1263 | 211661_x_at | 2821.53 | 11.40 | 1.24 | 1.43 | M80436 | Hs.46 | PTAFR |
| 1264 | 203117_s_at | 2821.81 | 11.40 | 1.24 | 1.42 | NM_014871 | Hs.273397 | USP52 |
| 1265 | 218639_s_at | 2821.95 | 11.39 | 1.26 | 1.45 | NM_025112 | Hs.288697 | MGC11349 |
| 1266 | 220940_at | 2823.75 | 11.42 | 1.22 | 1.39 | NM_025190 | Hs.389347 | KIAA1641 |
| 1267 | 219173_at | 2824.29 | 11.42 | 1.19 | 1.33 | NM_024957 | Hs.390817 | MYO15B |
| 1268 | 221541_at | 2824.83 | 11.43 | 1.21 | 1.37 | AL136861 | Hs.262958 | DKFZP434B044 |
| 1269 | 215470_at | 2825.82 | 11.45 | 1.24 | 1.42 | U21915 | Hs.398348 | — |
| 1270 | 212316_at | 2825.86 | 11.44 | 1.27 | 1.47 | AA502912 | Hs.292119 | NUP210 |
| 1271 | 201455_s_at | 2826.66 | 11.45 | 1.26 | 1.47 | AJ132583 | Hs.293007 | NPEPPS |
| 1272 | 203039_s_at | 2826.96 | 11.44 | 1.26 | 1.47 | NM_005006 | Hs.420563 | NDUFS1 |
| 1273 | 220865_s_at | 2829.02 | 11.49 | 1.25 | 1.44 | NM_014317 | Hs.279865 | TPRT |
| 1274 | 219317_at | 2831.01 | 11.51 | 1.21 | 1.36 | NM_007195 | Hs.438533 | POLI |
| 1275 | 204791_at | 2834.31 | 11.57 | 1.23 | 1.40 | NM_003297 | Hs.108301 | NR2C1 |
| 1276 | 209050_s_at | 2834.51 | 11.56 | 1.14 | 1.25 | AI421559 | Hs.106185 | RALGDS |
| 1277 | 202638_s_at | 2835.29 | 11.57 | 1.23 | 1.40 | NM_000201 | Hs.386467 | ICAM1 |
| 1278 | 214012_at | 2835.72 | 11.56 | 1.01 | 1.01 | BE551138 | Hs.436186 | ARTS-1 |
| 1279 | 208270_s_at | 2837.25 | 11.59 | 1.23 | 1.41 | NM_020216 | Hs.283667 | RNPEP |
| 1280 | 201585_s_at | 2841.04 | 11.67 | 1.25 | 1.43 | BG035151 | — | — |
| 1281 | 213348_at | 2843.69 | 11.71 | 1.11 | 1.19 | N33167 | Hs.106070 | CDKN1C |
| 1282 | 216191_s_at | 2847.34 | 11.78 | 0.97 | 0.96 | X72501 | Hs.2014 | TRDD3 /// TRD@ |
| 1283 | 211505_s_at | 2847.48 | 11.77 | 1.27 | 1.47 | AL136601 | Hs.6113 | STAU |
| 1284 | 202946_s_at | 2848.45 | 11.80 | 1.20 | 1.35 | NM_014962 | Hs.7935 | BTBD3 |
| 1285 | 204119_s_at | 2853.97 | 11.89 | 1.20 | 1.35 | U90339 | Hs.355533 | ADK |
| 1286 | 201894_s_at | 2856.92 | 11.94 | 1.25 | 1.45 | NM_001920 | Hs.250773 | SSR1 |
| 1287 | 215440_s_at | 2861.68 | 12.03 | 1.25 | 1.43 | AL523320 | Hs.184736 | BEXL1 |
| 1288 | 212689_s_at | 2861.87 | 12.02 | 1.26 | 1.47 | AA524505 | Hs.321707 | JMJD1A |
| 1289 | 205022_s_at | 2862.19 | 12.02 | 1.23 | 1.40 | NM_005197 | — | CHES1 |
| 1290 | 205197_s_at | 2864.98 | 12.07 | 1.20 | 1.34 | BE567813 | Hs.606 | ATP7A |
| 1291 | 210176_at | 2870.10 | 12.16 | 1.18 | 1.32 | AL050262 | Hs.111805 | TLR1 |
| 1292 | 212577_at | 2872.33 | 12.20 | 1.24 | 1.42 | AA868754 | Hs.8118 | KIAA0650 |
| 1293 | 201825_s_at | 2872.73 | 12.20 | 1.24 | 1.43 | AL572542 | Hs.238126 | CGI-49 |
| 1294 | 201189_s_at | 2872.93 | 12.20 | 1.24 | 1.43 | NM_002224 | Hs.77515 | ITPR3 |
| 1295 | 208819_at | 2873.46 | 12.20 | 1.24 | 1.42 | BC002977 | Hs.5947 | RAB8A |
| 1296 | 209510_at | 2873.60 | 12.20 | 1.23 | 1.41 | AF064801 | Hs.28285 | RNF139 |
| 1297 | 211725_s_at | 2873.87 | 12.19 | 1.20 | 1.35 | BC005884 | Hs.300825 | BID |
| 1298 | 205423_at | 2873.88 | 12.18 | 1.24 | 1.42 | NM_001127 | Hs.331602 | AP1B1 |
| 1299 | 217992_s_at | 2874.75 | 12.19 | 1.22 | 1.39 | NM_024329 | Hs.301342 | EFHD2 |
| 1300 | 217534_at | 2879.00 | 12.27 | 1.23 | 1.40 | AA845825 | — | — |
| 1301 | 208248_x_at | 2879.49 | 12.28 | 1.25 | 1.44 | NM_001642 | Hs.279518 | APLP2 |
| 1302 | 205069_s_at | 2879.92 | 12.28 | 1.21 | 1.36 | NM_015071 | Hs.132942 | ARHGAP26 |
| 1303 | 218418_s_at | 2880.40 | 12.28 | 1.23 | 1.41 | NM_015493 | Hs.284208 | ANKRD25 |
| 1304 | 209615_s_at | 2881.13 | 12.28 | 1.22 | 1.39 | U51120 | Hs.64056 | PAK1 |
| 1305 | 221704_s_at | 2882.87 | 12.31 | 1.21 | 1.36 | BC005882 | Hs.77870 | FLJ12750 |
| 1306 | 203297_s_at | 2882.99 | 12.30 | 1.21 | 1.37 | BG029530 | Hs.40154 | JARID2 |
| 1307 | 201483_s_at | 2883.38 | 12.30 | 1.21 | 1.37 | BC002802 | Hs.79058 | SUPT4H1 |
| 1308 | 218530_at | 2884.09 | 12.31 | 1.24 | 1.42 | NM_013241 | Hs.95231 | FHOD1 |
| 1309 | 204319_s_at | 2884.66 | 12.31 | 1.12 | 1.20 | NM_002925 | Hs.82280 | RGS10 |
| 1310 | 218284_at | 2885.73 | 12.31 | 1.26 | 1.46 | NM_015400 | — | — |
| 1311 | 212014_x_at | 2888.44 | 12.35 | 1.25 | 1.43 | AI493245 | Hs.306278 | CD44 |
| 1312 | 207723_s_at | 2889.28 | 12.36 | 1.05 | 1.08 | NM_002261 | — | KLRC3 |
| 1313 | 209025_s_at | 2889.66 | 12.37 | 1.25 | 1.45 | AF037448 | Hs.436376 | SYNCRIP |
| 1314 | 213931_at | 2890.60 | 12.38 | 1.22 | 1.39 | AI819238 | Hs.180919 | ID2 |
| 1315 | 201378_s_at | 2891.18 | 12.38 | 1.26 | 1.46 | NM_014847 | Hs.8127 | UBAP2L |
| 1316 | 201719_s_at | 2891.52 | 12.37 | 1.26 | 1.45 | NM_001431 | Hs.440387 | EPB41L2 |
| 1317 | 219202_at | 2895.61 | 12.45 | 1.21 | 1.37 | NM_024599 | — | — |
| 1318 | 204513_s_at | 2896.96 | 12.47 | 1.24 | 1.43 | NM_014800 | Hs.444695 | ELMO1 |
| 1319 | 208691_at | 2897.25 | 12.47 | 1.23 | 1.41 | BC001188 | Hs.185726 | TFRC |
| 1320 | 210993_s_at | 2898.40 | 12.48 | 1.16 | 1.28 | U54826 | Hs.388294 | SMAD1 |
| 1321 | 202043_s_at | 2899.82 | 12.50 | 1.23 | 1.40 | NM_004595 | Hs.449032 | SMS |
| 1322 | 205684_s_at | 2900.62 | 12.51 | 1.24 | 1.43 | NM_017925 | Hs.249591 | C9orf55 |
| 1323 | 204674_at | 2901.21 | 12.51 | 1.25 | 1.45 | NM_006152 | Hs.124922 | LRMP |
| 1324 | 204761_at | 2901.32 | 12.51 | 1.24 | 1.42 | NM_014688 | Hs.278526 | USP6NL |
| 1325 | 214502_at | 2901.71 | 12.50 | 1.17 | 1.29 | NM_021058 | Hs.519945 | HIST1H2BJ |
| 1326 | 221264_s_at | 2902.59 | 12.52 | 1.23 | 1.41 | NM_031214 | Hs.300624 | TARDBP |
| 1327 | 217635_s_at | 2903.17 | 12.52 | 1.25 | 1.45 | AA769006 | Hs.290921 | POLG |
| 1328 | 210266_s_at | 2903.43 | 12.52 | 1.25 | 1.44 | AF220137 | Hs.293044 | TRIM33 |
| 1329 | 204780_s_at | 2904.53 | 12.53 | 1.22 | 1.38 | AA164751 | Hs.82359 | TNFRSF6 |
| 1330 | 212041_at | 2909.73 | 12.62 | 1.22 | 1.38 | AL566172 | Hs.106876 | ATP6V0D1 |
| 1331 | 200752_s_at | 2912.47 | 12.68 | 1.23 | 1.40 | NM_005186 | Hs.356181 | CAPN1 |
| 1332 | 210136_at | 2912.87 | 12.68 | 1.19 | 1.34 | AW070431 | Hs.352898 | — |
| 1333 | 207691_x_at | 2913.87 | 12.69 | 1.24 | 1.42 | NM_001776 | Hs.444105 | ENTPD1 |
| 1334 | 209457_at | 2913.93 | 12.69 | 1.18 | 1.31 | U16996 | H.2128 | DUSP5 |
| 1335 | 218276_s_at | 2916.80 | 12.74 | 1.23 | 1.40 | NM_021818 | Hs.257341 | SAV1 |
| 1336 | 219284_at | 2917.62 | 12.75 | 1.20 | 1.35 | NM_024610 | Hs.29169 | HSPBAP1 |
| 1337 | 205826_at | 2917.94 | 12.75 | 0.73 | 0.60 | NM_003970 | Hs.443683 | MYOM2 |
| 1338 | 204686_at | 2918.02 | 12.74 | 1.18 | 1.31 | NM_005544 | Hs.390242 | IRS1 |
| 1339 | 210844_x_at | 2918.28 | 12.74 | 1.22 | 1.39 | D14705 | Hs.254321 | CTNNA1 |
| 1340 | 35974_at | 2923.71 | 12.84 | 1.26 | 1.46 | U10485 | Hs.124922 | LRMP |
| 1341 | 201690_s_at | 2924.99 | 12.86 | 1.26 | 1.46 | AA524023 | Hs.162089 | TPD52 |
| 1342 | 206088_at | 2926.54 | 12.88 | 1.21 | 1.37 | NM_014834 | Hs.433113 | KIAA0563 |
| 1343 | 200825_s_at | 2927.35 | 12.89 | 1.25 | 1.45 | NM_006389 | Hs.277704 | HYOU1 |
| 1344 | 201133_s_at | 2930.34 | 12.94 | 1.24 | 1.42 | AA142966 | Hs.224262 | PJA2 |
| 1345 | 207622_s_at | 2930.75 | 12.94 | 1.25 | 1.44 | NM_005692 | Hs.438823 | ABCF2 |
| 1346 | 204063_s_at | 2931.04 | 12.94 | 1.23 | 1.40 | NM_014683 | Hs.168762 | ULK2 |
| 1347 | 213483_at | 2932.52 | 12.96 | 1.25 | 1.44 | AK025679 | Hs.1191 | KIAA0073 |
| 1348 | 212384_at | 2933.41 | 12.96 | 1.21 | 1.36 | AI282485 | — | — |
| 1349 | 219528_s_at | 2933.54 | 12.95 | 1.21 | 1.36 | NM_022898 | Hs.57987 | BCL11B |
| 1350 | 201552_at | 2933.76 | 12.95 | 1.26 | 1.45 | NM_005561 | Hs.150101 | LAMP1 |
| 1351 | 214578_s_at | 2934.13 | 12.95 | 1.24 | 1.42 | AV683882 | Hs.306307 | ROCK1 |
| 1352 | 202899_s_at | 2936.19 | 12.99 | 1.22 | 1.38 | NM_003017 | Hs.405144 | SFRS3 |
| 1353 | 205950_s_at | 2937.07 | 13.00 | 1.23 | 1.41 | NM_001738 | Hs.23118 | CA1 |
| 1354 | 204398_s_at | 2937.63 | 13.01 | 1.23 | 1.40 | NM_012155 | Hs.24178 | EML2 |
| 1355 | 212040_at | 2938.40 | 13.02 | 1.20 | 1.35 | BG249599 | Hs.14894 | TGOLN2 |
| 1356 | 202368_s_at | 2939.07 | 13.03 | 1.25 | 1.45 | AI986461 | Hs.310230 | TRAM2 |
| 1357 | 203470_s_at | 2940.33 | 13.05 | 1.07 | 1.12 | AI433595 | Hs.77436 | PLEK |
| 1358 | 204143_s_at | 2941.09 | 13.05 | 1.22 | 1.39 | NM_017512 | Hs.475848 | HSRTSBETA |
| 1359 | 203869_at | 2944.46 | 13.11 | 1.26 | 1.46 | AK024318 | Hs.109268 | USP46 |
| 1360 | 220742_s_at | 2946.89 | 13.15 | 1.25 | 1.45 | NM_018297 | Hs.63657 | NGLY1 |
| 1361 | 201408_at | 2949.45 | 13.21 | 1.26 | 1.45 | W67887 | Hs.21537 | PPP1CB |
| 1362 | 213772_s_at | 2949.82 | 13.20 | 1.24 | 1.42 | BF196572 | Hs.133340 | GGA2 |
| 1363 | 217378_x_at | 2952.42 | 13.26 | 1.23 | 1.41 | X51887 | — | — |
| 1364 | 215012_at | 2952.82 | 13.26 | 1.21 | 1.36 | AU144775 | Hs.188662 | ZNF451 |
| 1365 | 201002_s_at | 2953.28 | 13.26 | 1.24 | 1.42 | U39361 | Hs.381025 | Kua-UEV /// |
| | | | | | | | | UBE2V1 |
| 1366 | 204417_at | 2955.89 | 13.31 | 1.24 | 1.41 | NM_000153 | Hs.408273 | GALC |
| 1367 | 207614_s_at | 2956.91 | 13.32 | 1.25 | 1.43 | NM_003592 | Hs.348153 | CUL1 |
| 1368 | 203185_at | 2956.98 | 13.31 | 1.23 | 1.41 | NM_014737 | Hs.80905 | RASSF2 |
| 1369 | 209696_at | 2957.72 | 13.32 | 1.21 | 1.37 | D26054 | Hs.360509 | FBP1 |
| 1370 | 216266_s_at | 2957.90 | 13.31 | 1.25 | 1.44 | AK025637 | Hs.94631 | ARFGEF1 |
| 1371 | 203481_at | 2958.34 | 13.31 | 1.25 | 1.44 | AI655902 | Hs.447458 | C10orf6 |
| 1372 | 215269_at | 2961.95 | 13.38 | 1.24 | 1.43 | AI922538 | Hs.438927 | TMEM1 |
| 1373 | 208815_x_at | 2962.57 | 13.38 | 1.25 | 1.44 | AB023420 | Hs.90093 | HSPA4 |
| 1374 | 206875_s_at | 2962.77 | 13.37 | 1.26 | 1.47 | NM_014720 | Hs.105751 | SLK |
| 1375 | 204293_at | 2964.18 | 13.39 | 1.21 | 1.37 | NM_000199 | Hs.31074 | SGSH |
| 1376 | 203853_s_at | 2966.29 | 13.43 | 1.20 | 1.35 | NM_012296 | Hs.30687 | GAB2 |
| 1377 | 215761_at | 2966.97 | 13.43 | 1.18 | 1.30 | AK000156 | Hs.200828 | RC3 |
| 1378 | 215535_s_at | 2968.44 | 13.46 | 1.23 | 1.40 | AF007145 | Hs.409230 | AGPAT1 |
| 1379 | 204507_s_at | 2970.62 | 13.50 | 1.16 | 1.27 | NM_000945 | Hs.280604 | PPP3R1 |
| 1380 | 202778_s_at | 2972.34 | 13.53 | 1.24 | 1.41 | NM_003453 | Hs.315241 | ZNF198 |
| 1381 | 204158_s_at | 2975.85 | 13.59 | 1.16 | 1.27 | NM_006019 | Hs.46465 | TCIRG1 |
| 1382 | 215268_at | 2976.35 | 13.59 | 1.24 | 1.43 | AW663712 | — | KIAA0754 |
| 1383 | 208919_s_at | 2979.42 | 13.65 | 1.20 | 1.34 | BC001709 | Hs.220324 | FLJ13052 |
| 1384 | 218669_at | 2981.25 | 13.68 | 1.26 | 1.45 | NM_021183 | Hs.225979 | RAP2C |
| 1385 | 200623_s_at | 2984.48 | 13.75 | 1.24 | 1.42 | NM_005184 | Hs.334330 | CALM3 |
| 1386 | 201862_s_at | 2987.35 | 13.80 | 1.24 | 1.43 | NM_004735 | Hs.512387 | LRRFIP1 |
| 1387 | 209471_s_at | 2987.77 | 13.80 | 1.25 | 1.44 | L00634 | Hs.356463 | FNTA |
| 1388 | 212252_at | 2989.15 | 13.82 | 1.22 | 1.39 | AA181179 | Hs.297343 | CAMKK2 |
| 1389 | 201747_s_at | 2989.70 | 13.83 | 1.25 | 1.43 | AI769566 | Hs.23978 | SAFB |
| 1390 | 204976_s_at | 2991.01 | 13.85 | 1.25 | 1.45 | AK023637 | Hs.433256 | AMMECR1 |
| 1391 | 220553_s_at | 2992.22 | 13.86 | 1.23 | 1.40 | NM_018333 | Hs.274337 | PRPF39 |
| 1392 | 202232_s_at | 2992.56 | 13.86 | 1.24 | 1.43 | NM_006360 | Hs.44893 | GA17 |
| 1393 | 202878_s_at | 2993.92 | 13.89 | 1.21 | 1.36 | NM_012072 | Hs.97199 | C1QR1 |
| 1394 | 209331_s_at | 2994.48 | 13.89 | 1.19 | 1.33 | AA723514 | Hs.42712 | MAX |
| 1395 | 201531_at | 2995.16 | 13.89 | 1.20 | 1.36 | NM_003407 | Hs.343586 | ZFP36 |
| 1396 | 217833_at | 2995.45 | 13.89 | 1.25 | 1.45 | AL520908 | Hs.436376 | SYNCRIP |
| 1397 | 204411_at | 2995.48 | 13.88 | 1.25 | 1.44 | NM_017596 | Hs.169182 | KIF21B |
| 1398 | 222303_at | 2995.92 | 13.88 | 1.23 | 1.40 | AV700891 | — | — |
| 1399 | 219984_s_at | 2996.25 | 13.88 | 1.17 | 1.30 | NM_020386 | Hs.36761 | HRASLS |
| 1400 | 211106_at | 2996.91 | 13.88 | 1.21 | 1.36 | AF064804 | Hs.410088 | SUPT3H |
| 1401 | 202519_at | 2997.64 | 13.88 | 1.24 | 1.42 | NM_014938 | Hs.528339 | MONDOA |
| 1402 | 219806_s_at | 2998.14 | 13.89 | 1.17 | 1.30 | NM_020179 | Hs.416456 | FN5 |
| 1403 | 215087_at | 2998.52 | 13.89 | 1.21 | 1.37 | AL109730 | Hs.17936 | DKFZP434H132 |
| 1404 | 218977_s_at | 2999.54 | 13.90 | 1.25 | 1.43 | NM_017846 | Hs.266935 | SECP43 |
| 1405 | 200041_s_at | 3001.67 | 13.94 | 1.23 | 1.41 | NM_004640 | — | — |
| 1406 | 217743_s_at | 3002.81 | 13.96 | 1.26 | 1.45 | NM_018247 | Hs.108530 | TMEM30A |
| 1407 | 207008_at | 3003.70 | 13.98 | 1.17 | 1.29 | NM_001557 | Hs.846 | IL8RB |
| 1408 | 212943_at | 3004.28 | 13.98 | 1.24 | 1.42 | AB011100 | Hs.30656 | KIAA0528 |
| 1409 | 209200_at | 3004.28 | 13.97 | 1.23 | 1.41 | AL536517 | Hs.368950 | MEF2C |
| 1410 | 208631_s_at | 3006.58 | 14.01 | 1.23 | 1.41 | U04627 | Hs.75860 | HADHA |
| 1411 | 212825_at | 3006.63 | 14.00 | 1.25 | 1.45 | AI357401 | Hs.443881 | PAXIP1L |
| 1412 | 217925_s_at | 3007.56 | 14.01 | 1.23 | 1.40 | NM_022758 | Hs.25999 | C6orf106 |
| 1413 | 201494_at | 3008.74 | 14.03 | 1.24 | 1.42 | NM_005040 | Hs.314089 | PRCP |
| 1414 | 208810_at | 3012.17 | 14.10 | 1.24 | 1.42 | AF080569 | Hs.181195 | DNAJB6 |
| 1415 | 204000_at | 3017.27 | 14.20 | 1.21 | 1.37 | NM_016194 | Hs.155090 | GNB5 |
| 1416 | 202647_s_at | 3018.18 | 14.21 | 1.23 | 1.40 | NM_002524 | Hs.260523 | NRAS |
| 1417 | 219453_at | 3019.08 | 14.22 | 1.23 | 1.40 | NM_024731 | Hs.222731 | C16orf44 |
| 1418 | 220370_s_at | 3019.45 | 14.22 | 1.20 | 1.35 | NM_025090 | Hs.11387 | USP36 |
| 1419 | 210623_at | 3019.91 | 14.23 | 1.24 | 1.42 | BC001372 | Hs.351296 | LOC51035 |
| 1420 | 201742_x_at | 3020.10 | 14.22 | 1.24 | 1.42 | NM_006924 | Hs.68714 | SFRS1 |
| 1421 | 218111_s_at | 3020.44 | 14.22 | 1.24 | 1.43 | NM_018686 | Hs.311346 | CMAS |
| 1422 | 217371_s_at | 3020.44 | 14.21 | 1.23 | 1.41 | Y09908 | Hs.528402 | IL15 |
| 1423 | 219503_s_at | 3020.69 | 14.21 | 1.15 | 1.26 | NM_018306 | Hs.263876 | FLJ11036 |
| 1424 | 200732_s_at | 3020.74 | 14.20 | 1.24 | 1.42 | AL578310 | Hs.227777 | PTP4A1 |
| 1425 | 210156_s_at | 3021.17 | 14.20 | 1.21 | 1.36 | D25547 | Hs.79137 | PCMT1 |
| 1426 | 212836_at | 3022.30 | 14.21 | 1.24 | 1.43 | D26018 | Hs.82502 | POLD3 |
| 1427 | 203935_at | 3022.39 | 14.21 | 1.23 | 1.41 | NM_001105 | Hs.150402 | ACVR1 |
| 1428 | 212810_s_at | 3023.64 | 14.22 | 1.20 | 1.35 | W72527 | Hs.323878 | SLC1A4 |
| 1429 | 219067_s_at | 3024.05 | 14.23 | 1.25 | 1.44 | NM_017615 | Hs.258798 | C10orf86 |
| 1430 | 221555_x_at | 3026.27 | 14.27 | 1.17 | 1.29 | AU145941 | Hs.22116 | CDC14B |
| 1431 | 212521_s_at | 3026.75 | 14.27 | 1.24 | 1.42 | BE568219 | Hs.78746 | PDE8A |
| 1432 | 209626_s_at | 3029.50 | 14.33 | 1.22 | 1.39 | AI202969 | Hs.197955 | OSBPL3 |
| 1433 | 209697_at | 3029.81 | 14.33 | 1.21 | 1.36 | BC004864 | — | — |
| 1434 | 202749_at | 3030.59 | 14.34 | 1.17 | 1.29 | NM_004627 | Hs.198308 | WRB |
| 1435 | 220521_s_at | 3033.14 | 14.39 | 1.25 | 1.44 | NM_017974 | Hs.419213 | APG16L |
| 1436 | 211475_s_at | 3033.25 | 14.38 | 1.22 | 1.38 | AF116273 | Hs.377484 | BAG1 |
| 1437 | 212640_at | 3033.77 | 14.38 | 1.22 | 1.39 | AV712602 | Hs.5957 | PTPLB |
| 1438 | 209286_at | 3034.72 | 14.39 | 1.22 | 1.39 | AI754416 | Hs.352554 | CDC42EP3 |
| 1439 | 209191_at | 3036.65 | 14.42 | 1.20 | 1.35 | BC002654 | Hs.274398 | MGC4083 |
| 1440 | 200973_s_at | 3036.94 | 14.42 | 1.23 | 1.40 | NM_005724 | Hs.259212 | TM4SF8 |
| 1441 | 220001_at | 3038.17 | 14.44 | 1.20 | 1.35 | NM_012387 | Hs.397050 | PADI4 |
| 1442 | 221725_at | 3038.36 | 14.43 | 1.23 | 1.40 | AI962978 | Hs.446403 | WASF2 |
| 1443 | 205098_at | 3038.46 | 14.42 | 1.18 | 1.30 | AI421071 | — | — |
| 1444 | 217040_x_at | 3038.50 | 14.42 | 1.22 | 1.39 | AB025355 | Hs.95582 | SOX15 |
| 1445 | 210418_s_at | 3039.33 | 14.43 | 1.24 | 1.43 | AF023265 | Hs.436405 | IDH3B |
| 1446 | 209683_at | 3039.46 | 14.42 | 1.23 | 1.40 | AA243659 | Hs.4863 | FAM49A |
| 1447 | 40472_at | 3039.64 | 14.42 | 1.24 | 1.43 | AF007155 | Hs.352614 | LOC254531 |
| 1448 | 202973_x_at | 3039.81 | 14.41 | 1.24 | 1.42 | NM_014883 | Hs.442818 | FAM13A1 |
| 1449 | 206296_x_at | 3040.30 | 14.41 | 1.22 | 1.38 | NM_007181 | Hs.95424 | MAP4K1 |
| 1450 | 214308_s_at | 3040.87 | 14.41 | 1.07 | 1.11 | AI478172 | Hs.116992 | HGD |
| 1451 | 200064_at | 3041.07 | 14.41 | 1.24 | 1.43 | AF275719 | Hs.74335 | HSPCB |
| 1452 | 202548_s_at | 3041.69 | 14.41 | 1.24 | 1.42 | NM_003899 | Hs.172813 | ARHGEF7 |
| 1453 | 209846_s_at | 3043.39 | 14.45 | 1.16 | 1.27 | BC002832 | Hs.376046 | BTN3A2 |
| 1454 | 221778_at | 3045.12 | 14.47 | 1.24 | 1.42 | BE217882 | Hs.222707 | KIAA1718 |
| 1455 | 201571_s_at | 3048.91 | 14.57 | 1.22 | 1.38 | AI656493 | Hs.76894 | DCTD |
| 1456 | 204427_s_at | 3049.97 | 14.58 | 1.25 | 1.44 | NM_006815 | Hs.75914 | RNP24 |
| 1457 | 208899_x_at | 3050.74 | 14.60 | 1.22 | 1.38 | AF100741 | Hs.272630 | ATP6V1D |
| 1458 | 202770_s_at | 3051.51 | 14.60 | 1.20 | 1.35 | NM_004354 | Hs.13291 | CCNG2 |
| 1459 | 212360_at | 3051.79 | 14.60 | 1.17 | 1.30 | AI916249 | Hs.82927 | AMPD2 |
| 1460 | 219259_at | 3051.86 | 14.59 | 1.23 | 1.40 | NM_022367 | Hs.408846 | SEMA4A |
| 1461 | 202083_s_at | 3053.82 | 14.63 | 1.19 | 1.33 | AI017770 | Hs.75232 | SEC14L1 |
| 1462 | 206488_s_at | 3054.99 | 14.64 | 1.19 | 1.33 | NM_000072 | Hs.443120 | CD36 |
| 1463 | 203130_s_at | 3055.17 | 14.63 | 1.23 | 1.40 | NM_004522 | Hs.6641 | KIF5C |
| 1464 | 37145_at | 3055.53 | 14.63 | 1.00 | 1.01 | M85276 | Hs.105806 | GNLY |
| 1465 | 213061_s_at | 3058.32 | 14.69 | 1.25 | 1.44 | AA643304 | Hs.351573 | NTAN1 |
| 1466 | 205076_s_at | 3059.00 | 14.70 | 1.23 | 1.40 | NM_006697 | Hs.425144 | CRA |
| 1467 | 209339_at | 3060.48 | 14.72 | 1.22 | 1.38 | U76248 | Hs.20191 | SIAH2 |
| 1468 | 208863_s_at | 3060.54 | 14.71 | 1.25 | 1.43 | M72709 | Hs.68714 | SFRS1 |
| 1469 | 205345_at | 3062.01 | 14.74 | 1.23 | 1.41 | NM_000465 | Hs.54089 | BARD1 |
| 1470 | 202651_at | 3063.79 | 14.79 | 1.23 | 1.41 | NM_014873 | Hs.528724 | FAM34A |
| 1471 | 221741_s_at | 3063.83 | 14.78 | 1.22 | 1.38 | AL096828 | Hs.11747 | YTHDF1 |
| 1472 | 203064_s_at | 3064.11 | 14.78 | 1.20 | 1.34 | NM_004514 | Hs.439387 | FOXK2 |
| 1473 | 219922_s_at | 3064.27 | 14.77 | 1.19 | 1.34 | NM_021070 | Hs.289019 | LTBP3 |
| 1474 | 206111_at | 3065.98 | 14.81 | 1.27 | 1.48 | NM_002934 | Hs.728 | RNASE2 |
| 1475 | 209273_s_at | 3066.21 | 14.80 | 1.20 | 1.34 | BG387555 | — | — |
| 1476 | 212516_at | 3067.33 | 14.81 | 1.20 | 1.34 | AB018325 | Hs.192190 | CENTD2 |
| 1477 | 205480_s_at | 3067.74 | 14.81 | 1.21 | 1.37 | NM_006759 | Hs.417361 | UGP2 |
| 1478 | 208092_s_at | 3068.84 | 14.83 | 1.18 | 1.31 | NM_030797 | Hs.4863 | FAM49A |
| 1479 | 221724_s_at | 3068.85 | 14.82 | 1.24 | 1.42 | AF200738 | Hs.115515 | CLECSF6 |
| 1480 | 217749_at | 3069.26 | 14.82 | 1.24 | 1.43 | NM_016128 | Hs.368056 | COPG |
| 1481 | 219599_at | 3071.36 | 14.86 | 1.08 | 1.14 | NM_018507 | — | — |
| 1482 | 207072_at | 3071.38 | 14.85 | 1.10 | 1.17 | NM_003853 | Hs.158315 | IL18RAP |
| 1483 | 215605_at | 3071.39 | 14.84 | 1.21 | 1.37 | AU145806 | Hs.446678 | NCOA2 |
| 1484 | 209012_at | 3071.80 | 14.84 | 1.22 | 1.38 | AV718192 | Hs.367689 | TRIO |
| 1485 | 210706_s_at | 3071.95 | 14.83 | 1.24 | 1.42 | BC000213 | Hs.30524 | RNF24 |
| 1486 | 204072_s_at | 3071.96 | 14.82 | 1.23 | 1.40 | NM_023037 | Hs.390874 | 13CDNA73 |
| 1487 | 212287_at | 3072.79 | 14.84 | 1.22 | 1.39 | BF382924 | Hs.335118 | JJAZ1 |
| 1488 | 202813_at | 3074.01 | 14.85 | 1.22 | 1.38 | NM_005646 | Hs.151518 | TARBP1 |
| 1489 | 210417_s_at | 3076.11 | 14.91 | 1.24 | 1.42 | U81802 | Hs.154846 | PIK4C8 |
| 1490 | 219434_at | 3079.33 | 14.97 | 1.21 | 1.36 | NM_018643 | Hs.283022 | TREM1 |
| 1491 | 221143_at | 3079.79 | 14.96 | 1.18 | 1.32 | NM_013347 | Hs.226483 | RPA4 |
| 1492 | 206959_s_at | 3081.69 | 15.00 | 1.23 | 1.40 | NM_023011 | Hs.399740 | UPF3A |
| 1493 | 212693_at | 3082.65 | 15.01 | 1.24 | 1.43 | BE670928 | Hs.181334 | MDN1 |
| 1494 | 213440_at | 3083.65 | 15.02 | 1.23 | 1.41 | AL530264 | Hs.227327 | RAB1A |
| 1495 | 202437_s_at | 3084.39 | 15.03 | 1.13 | 1.23 | NM_000104 | Hs.154654 | CYP1B1 |
| 1496 | 212001_at | 3084.58 | 15.03 | 1.22 | 1.39 | AV738039 | Hs.134093 | SFRS14 |
| 1497 | 220066_at | 3084.65 | 15.02 | 1.22 | 1.39 | NM_022162 | Hs.135201 | CARD15 |
| 1498 | 213190_at | 3090.46 | 15.13 | 1.24 | 1.42 | R61519 | Hs.185807 | COG7 |
| 1499 | 201626_at | 3092.26 | 15.16 | 1.22 | 1.39 | BG292233 | Hs.416385 | INSIG1 |
| 1500 | 202817_s_at | 3094.11 | 15.19 | 1.24 | 1.42 | NM_005637 | Hs.404263 | SS18 |
| 1501 | 200813_s_at | 3094.57 | 15.19 | 1.24 | 1.42 | BE256969 | Hs.77318 | PAFAH1B1 |
| 1502 | 204629_at | 3094.61 | 15.18 | 1.21 | 1.37 | NM_013327 | Hs.8836 | PARVB |
| 1503 | 212966_at | 3096.77 | 15.24 | 1.20 | 1.36 | AL043112 | Hs.109445 | HIC2 |
| 1504 | 211922_s_at | 3099.69 | 15.29 | 1.21 | 1.37 | AY028632 | Hs.395771 | CAT |
| 1505 | 204204_at | 3099.86 | 15.29 | 1.23 | 1.40 | NM_001860 | Hs.24030 | SLC31A2 |
| 1506 | 212269_s_at | 3100.23 | 15.29 | 1.21 | 1.37 | AJ010089 | Hs.389037 | MCM3AP |
| 1507 | 215714_s_at | 3101.96 | 15.32 | 1.24 | 1.42 | AF254822 | Hs.78202 | SMARCA4 |
| 1508 | 203615_x_at | 3102.24 | 15.31 | 1.21 | 1.37 | NM_001055 | Hs.142 | SULT1A1 |
| 1509 | 221675_s_at | 3102.30 | 15.30 | 1.22 | 1.38 | AF195624 | Hs.225567 | CHPT1 |
| 1510 | 204156_at | 3104.79 | 15.36 | 1.24 | 1.43 | AA044154 | Hs.444909 | KIAA0999 |
| 1511 | 215287_at | 3105.00 | 15.35 | 1.25 | 1.44 | AA975427 | Hs.128434 | — |
| 1512 | 201122_x_at | 3108.04 | 15.42 | 1.15 | 1.25 | BC000751 | — | — |
| 1513 | 209142_s_at | 3109.82 | 15.46 | 1.20 | 1.34 | BC002775 | Hs.78563 | UBE2G1 |
| 1514 | 220330_s_at | 3110.03 | 15.45 | 1.20 | 1.36 | NM_022136 | Hs.221851 | SAMSN1 |
| 1515 | 212595_s_at | 3110.47 | 15.45 | 1.21 | 1.37 | AL534321 | Hs.369761 | DAZAP2 |
| 1516 | 203243_s_at | 3111.81 | 15.48 | 1.23 | 1.41 | NM_006457 | Hs.154103 | LIM |
| 1517 | 215513_at | 3112.14 | 15.48 | 1.15 | 1.25 | AF241534 | — | HYMAI |
| 1518 | 219355_at | 3115.24 | 15.55 | 1.23 | 1.40 | NM_018015 | Hs.274267 | FLJ10178 |
| 1519 | 214431_at | 3115.62 | 15.55 | 1.20 | 1.36 | NM_003875 | Hs.144057 | GMPS |
| 1520 | 201376_s_at | 3120.30 | 15.64 | 1.21 | 1.37 | AI591354 | Hs.808 | HNRPF |
| 1521 | 218462_at | 3120.91 | 15.64 | 1.24 | 1.43 | NM_025065 | Hs.287863 | RPF1 |
| 1522 | 201767_s_at | 3120.98 | 15.63 | 1.23 | 1.41 | NM_018127 | Hs.12124 | ELAC2 |
| 1523 | 209239_at | 3122.08 | 15.64 | 1.24 | 1.42 | M55643 | Hs.160557 | NFKB1 |
| 1524 | 205698_s_at | 3124.02 | 15.67 | 1.23 | 1.41 | NM_002758 | Hs.256924 | MAP2K6 |
| 1525 | 204306_s_at | 3125.08 | 15.69 | 1.23 | 1.40 | NM_004357 | Hs.512857 | CD151 |
| 1526 | 209049_s_at | 3125.34 | 15.68 | 1.19 | 1.33 | BC001004 | Hs.37372 | PRKCBP1 |
| 1527 | 212798_s_at | 3127.21 | 15.71 | 1.24 | 1.42 | AK001389 | Hs.112605 | ANKMY2 |
| 1528 | 217555_at | 3127.32 | 15.71 | 1.24 | 1.41 | AI042030 | Hs.211602 | SMC1L1 |
| 1529 | 208640_at | 3128.35 | 15.72 | 1.22 | 1.39 | BG292367 | Hs.413812 | RAC1 |
| 1530 | 203116_s_at | 3128.77 | 15.72 | 1.23 | 1.41 | NM_000140 | Hs.443610 | FECH |
| 1531 | 209412_at | 3130.17 | 15.74 | 1.24 | 1.43 | U61500 | Hs.438927 | TMEM1 |
| 1532 | 201328_at | 3131.09 | 15.75 | 1.24 | 1.42 | AL575509 | Hs.292477 | ETS2 |
| 1533 | 218502_s_at | 3131.91 | 15.76 | 1.18 | 1.32 | NM_014112 | Hs.26102 | TRPS1 |
| 1534 | 217610_at | 3133.63 | 15.80 | 1.12 | 1.21 | AL047879 | Hs.293865 | — |
| 1535 | 215392_at | 3134.37 | 15.81 | 1.23 | 1.41 | AU148154 | Hs.298014 | — |
| 1536 | 206983_at | 3135.86 | 15.84 | 1.23 | 1.41 | NM_004367 | Hs.46468 | CCR6 |
| 1537 | 208485_x_at | 3138.00 | 15.88 | 1.22 | 1.39 | NM_003879 | Hs.355724 | CFLAR |
| 1538 | 215499_at | 3139.19 | 15.89 | 1.20 | 1.34 | AA780381 | Hs.180533 | MAP2K3 |
| 1539 | 218098_at | 3139.76 | 15.89 | 1.25 | 1.44 | AL121903 | Hs.310323 | ARFGEF2 |
| 1540 | 213305_s_at | 3140.86 | 15.91 | 1.23 | 1.40 | L42375 | Hs.249955 | PPP2R5C |
| 1541 | 204970_s_at | 3141.25 | 15.91 | 1.22 | 1.39 | NM_002359 | Hs.252229 | MAFG |
| 1542 | 201751_at | 3141.28 | 15.90 | 1.23 | 1.41 | NM_014876 | Hs.3094 | KIAA0063 |
| 1543 | 218403_at | 3141.95 | 15.90 | 1.19 | 1.34 | NM_016399 | Hs.69499 | HSPC132 |
| 1544 | 201938_at | 3143.70 | 15.94 | 1.14 | 1.25 | NM_004642 | Hs.433201 | CDK2AP1 |
| 1545 | 208886_at | 3144.00 | 15.94 | 1.17 | 1.29 | BC000145 | Hs.226117 | H1F0 |
| 1546 | 216274_s_at | 3146.73 | 15.99 | 1.23 | 1.41 | N99438 | Hs.9534 | SPC18 |
| 1547 | 201604_s_at | 3147.31 | 16.00 | 1.22 | 1.39 | NM_002480 | Hs.377908 | PPP1R12A |
| 1548 | 213434_at | 3147.94 | 16.01 | 1.20 | 1.34 | H95263 | Hs.99865 | EPIM |
| 1549 | 221830_at | 3148.70 | 16.01 | 1.21 | 1.36 | AI302106 | Hs.48554 | RAP2A |
| 1550 | 208752_x_at | 3148.86 | 16.00 | 1.22 | 1.39 | AI888672 | Hs.419776 | NAP1L1 |
| 1551 | 219242_at | 3149.74 | 16.02 | 1.23 | 1.41 | NM_025180 | Hs.443301 | Cep63 |
| 1552 | 207104_x_at | 3150.74 | 16.04 | 1.21 | 1.36 | NM_006669 | Hs.149924 | LILRB1 |
| 1553 | 214953_s_at | 3152.73 | 16.08 | 1.09 | 1.15 | X06989 | Hs.177486 | APP |
| 1554 | 210285_x_at | 3152.93 | 16.08 | 1.20 | 1.36 | BC000383 | Hs.446091 | WTAP |
| 1555 | 212557_at | 3153.43 | 16.09 | 1.23 | 1.40 | AB011148 | Hs.188662 | ZNF451 |
| 1556 | 214511_x_at | 3153.80 | 16.08 | 1.01 | 1.02 | L03419 | Hs.77424 | FCGR1A |
| 1557 | 202445_s_at | 3153.92 | 16.07 | 1.23 | 1.40 | NM_024408 | Hs.8121 | NOTCH2 |
| 1558 | 201561_s_at | 3156.73 | 16.13 | 1.24 | 1.42 | NM_014944 | Hs.29665 | CLSTN1 |
| 1559 | 220768_s_at | 3158.30 | 16.16 | 1.22 | 1.39 | NM_004384 | Hs.129206 | CSNK1G3 |
| 1560 | 221751_at | 3158.62 | 16.15 | 1.24 | 1.43 | AL565516 | Hs.388400 | PANK3 |
| 1561 | 220467_at | 3161.65 | 16.22 | 1.23 | 1.41 | NM_025032 | Hs.523544 | FLJ21272 |
| 1562 | 201722_s_at | 3162.38 | 16.23 | 1.24 | 1.42 | AV692127 | Hs.277324 | GALNT1 |
| 1563 | 205401_at | 3163.66 | 16.25 | 1.22 | 1.39 | NM_003659 | Hs.407933 | AGPS |
| 1564 | 200969_at | 3164.65 | 16.26 | 1.22 | 1.38 | BG107676 | Hs.439874 | SERP1 |
| 1565 | 200743_s_at | 3167.50 | 16.32 | 1.22 | 1.38 | NM_000391 | Hs.429658 | CLN2 |
| 1566 | 200776_s_at | 3169.89 | 16.37 | 1.22 | 1.38 | AL518328 | Hs.355983 | BZW1 |
| 1567 | 200867_at | 3170.05 | 16.36 | 1.24 | 1.42 | AL031685 | Hs.144949 | ZNF313 |
| 1568 | 215390_at | 3171.67 | 16.40 | 1.23 | 1.40 | AU147194 | Hs.494744 | — |
| 1569 | 206986_at | 3171.91 | 16.39 | 1.22 | 1.38 | AB007422 | — | — |
| 1570 | 202430_s_at | 3172.21 | 16.40 | 1.18 | 1.31 | NM_021105 | Hs.348478 | PLSCR1 |
| 1571 | 208700_s_at | 3172.36 | 16.39 | 1.22 | 1.39 | L12711 | Hs.89643 | TKT |
| 1572 | 222156_x_at | 3175.00 | 16.44 | 1.24 | 1.43 | AK022459 | Hs.259326 | CCPG1 |
| 1573 | 209571_at | 3175.59 | 16.44 | 1.20 | 1.36 | U03644 | Hs.89421 | CIR |
| 1574 | 209911_x_at | 3176.06 | 16.45 | 1.02 | 1.03 | BC002842 | Hs.180779 | HIST1H2BD |
| 1575 | 200771_at | 3176.53 | 16.44 | 1.16 | 1.28 | NM_002293 | Hs.432855 | LAMC1 |
| 1576 | 202464_s_at | 3177.77 | 16.46 | 1.11 | 1.18 | NM_004566 | Hs.195471 | PFKFB3 |
| 1577 | 208923_at | 3179.04 | 16.48 | 1.21 | 1.37 | BC005097 | Hs.26704 | CYFIP1 |
| 1578 | 38487_at | 3180.57 | 16.52 | 1.16 | 1.27 | D87433 | Hs.301989 | STAB1 |
| 1579 | 212200_at | 3180.64 | 16.51 | 1.21 | 1.37 | AK025933 | Hs.524874 | KIAA0692 |
| 1580 | 211368_s_at | 3180.83 | 16.51 | 1.20 | 1.35 | U13700 | Hs.2490 | CASP1 |
| 1581 | 202487_s_at | 3181.28 | 16.51 | 1.21 | 1.36 | NM_012412 | Hs.301005 | H2AFV |
| 1582 | 204004_at | 3183.04 | 16.54 | 1.22 | 1.39 | AI336206 | Hs.406074 | PAWR |
| 1583 | 201727_s_at | 3183.66 | 16.54 | 1.21 | 1.37 | NM_001419 | Hs.184492 | ELAVL1 |
| 1584 | 203582_s_at | 3183.81 | 16.53 | 1.22 | 1.38 | NM_004578 | Hs.296169 | RAB4A |
| 1585 | 214307_at | 3184.34 | 16.54 | 0.99 | 0.99 | AI478172 | Hs.116992 | HGD |
| 1586 | 216060_s_at | 3186.04 | 16.56 | 1.21 | 1.37 | AK021890 | Hs.356537 | DAAM1 |
| 1587 | 215358_x_at | 3187.75 | 16.60 | 1.20 | 1.35 | AK026980 | — | ZNF37B |
| 1588 | 213906_at | 3187.88 | 16.59 | 1.17 | 1.30 | AW592266 | Hs.300592 | MYBL1 |
| 1589 | 222244_s_at | 3188.68 | 16.60 | 1.22 | 1.39 | AK000749 | Hs.52184 | FLJ20618 |
| 1590 | 200782_at | 3191.40 | 16.66 | 1.23 | 1.41 | NM_001154 | Hs.145741 | ANXA5 |
| 1591 | 214339_s_at | 3193.75 | 16.71 | 1.23 | 1.40 | AA744529 | Hs.95424 | MAP4K1 |
| 1592 | 221640_s_at | 3194.48 | 16.71 | 1.19 | 1.34 | AF274972 | Hs.438986 | LRDD |
| 1593 | 200663_at | 3194.58 | 16.71 | 1.20 | 1.35 | NM_001780 | Hs.445570 | CD63 |
| 1594 | 201876_at | 3194.69 | 16.70 | 1.24 | 1.42 | NM_000305 | Hs.165598 | PON2 |
| 1595 | 213238_at | 3195.30 | 16.71 | 1.22 | 1.38 | AI478147 | Hs.437241 | ATP10D |
| 1596 | 202377_at | 3196.61 | 16.73 | 1.21 | 1.36 | AW026535 | Hs.23581 | LEPR /// OBRGRP |
| 1597 | 200011_s_at | 3197.09 | 16.73 | 1.23 | 1.40 | NM_001659 | Hs.119177 | ARF3 |
| 1598 | 206483_at | 3197.26 | 16.72 | 1.16 | 1.28 | NM_012472 | — | — |
| 1599 | 221806_s_at | 3200.35 | 16.79 | 1.23 | 1.40 | BF590997 | Hs.405917 | FLJ10707 |
| 1600 | 212465_at | 3202.09 | 16.83 | 1.22 | 1.39 | AA524500 | Hs.321766 | C14orf154 |
| 1601 | 212805_at | 3202.89 | 16.83 | 1.21 | 1.36 | AB002365 | Hs.23311 | KIAA0367 |
| 1602 | 203164_at | 3202.98 | 16.83 | 1.22 | 1.39 | BE464756 | Hs.285176 | SLC33A1 |
| 1603 | 211352_s_at | 3204.23 | 16.85 | 1.22 | 1.39 | U80737 | Hs.382168 | NCOA3 |
| 1604 | 201170_s_at | 3205.17 | 16.86 | 1.20 | 1.36 | NM_003670 | Hs.171825 | BHLHB2 |
| 1605 | 205052_at | 3205.30 | 16.86 | 1.24 | 1.42 | NM_001698 | Hs.81886 | AUH |
| 1606 | 220416_at | 3205.63 | 16.86 | 1.23 | 1.40 | NM_024837 | Hs.313841 | ATP8B4 |
| 1607 | 213149_at | 3206.16 | 16.86 | 1.21 | 1.36 | AW299740 | Hs.115285 | DLAT |
| 1608 | 218506_x_at | 3206.35 | 16.86 | 1.19 | 1.33 | NM_018459 | — | — |
| 1609 | 202379_s_at | 3207.35 | 16.87 | 1.18 | 1.32 | AI361805 | Hs.369815 | NKTR |
| 1610 | 220200_s_at | 3208.36 | 16.88 | 1.23 | 1.40 | NM_020382 | Hs.287493 | SET8 |
| 1611 | 220615_s_at | 3209.23 | 16.91 | 1.21 | 1.37 | NM_018099 | Hs.134497 | MLSTD1 |
| 1612 | 204735_at | 3209.43 | 16.96 | 1.20 | 1.35 | NM_006202 | Hs.89901 | PDE4A |
| 1613 | 221246_x_at | 3209.59 | 16.89 | 1.19 | 1.33 | NM_018274 | Hs.439442 | TNS |
| 1614 | 221498_at | 3210.26 | 16.89 | 1.23 | 1.40 | BF939727 | Hs.67619 | SNX27 |
| 1615 | 203087_s_at | 3211.08 | 16.90 | 1.17 | 1.29 | NM_004520 | Hs.113319 | KIF2 |
| 1616 | 210542_s_at | 3211.34 | 16.89 | 1.22 | 1.39 | BC000585 | Hs.113657 | SLCO3A1 |
| 1617 | 210942_s_at | 3212.37 | 16.91 | 1.16 | 1.27 | AB022918 | Hs.440913 | SIAT10 |
| 1618 | 215090_x_at | 3212.83 | 16.91 | 1.21 | 1.37 | AK021884 | — | FLJ11822 |
| 1619 | 219925_at | 3213.18 | 16.91 | 1.21 | 1.36 | NM_007167 | Hs.434283 | ZNF258 |
| 1620 | 220410_s_at | 3213.61 | 16.91 | 1.22 | 1.39 | NM_018627 | — | — |
| 1621 | 209791_at | 3215.93 | 16.95 | 1.17 | 1.29 | AL049569 | Hs.33455 | PADI2 |
| 1622 | 219675_s_at | 3218.08 | 16.99 | 1.22 | 1.38 | NM_025076 | Hs.288158 | UXS1 |
| 1623 | 218164_at | 3218.96 | 17.00 | 1.17 | 1.29 | NM_022827 | Hs.103147 | SSP411 |
| 1624 | 212260_at | 3219.14 | 17.00 | 1.24 | 1.41 | AL045800 | Hs.334871 | TNRC15 |
| 1625 | 213295_at | 3222.15 | 17.06 | 1.24 | 1.42 | AA555096 | Hs.386952 | CYLD |
| 1626 | 218238_at | 3222.94 | 17.07 | 1.22 | 1.39 | NM_012341 | Hs.215766 | GTPBP4 |
| 1627 | 200712_s_at | 3223.09 | 17.06 | 1.21 | 1.37 | AI633566 | Hs.408754 | MAPRE1 |
| 1628 | 204334_at | 3224.54 | 17.09 | 1.22 | 1.39 | AA488672 | Hs.436708 | KLF7 |
| 1629 | 222061_at | 3225.10 | 17.09 | 1.23 | 1.40 | AA700015 | Hs.75626 | CD58 |
| 1630 | 214522_x_at | 3225.11 | 17.08 | 1.17 | 1.29 | NM_021065 | Hs.239458 | HIST1H2AD /// |
| | | | | | | | | HIST1H3D |
| 1631 | 205212_s_at | 3225.61 | 17.08 | 1.21 | 1.36 | NM_014716 | Hs.337242 | CENTB1 |
| 1632 | 213590_at | 3225.80 | 17.07 | 1.21 | 1.37 | AA705628 | Hs.90911 | SLC16A5 |
| 1633 | 221501_x_at | 3226.54 | 17.09 | 1.20 | 1.34 | AF229069 | Hs.449809 | LOC339047 |
| 1634 | 208325_s_at | 3226.59 | 17.08 | 0.98 | 0.97 | NM_006738 | Hs.350631 | AKAP13 |
| 1635 | 218272_at | 3226.73 | 17.07 | 1.18 | 1.32 | NM_017931 | Hs.435832 | FLJ20699 |
| 1636 | 206342_x_at | 3226.80 | 17.06 | 1.20 | 1.35 | NM_006123 | Hs.303154 | IDS |
| 1637 | 212156_at | 3226.88 | 17.05 | 1.22 | 1.38 | AA812224 | Hs.417482 | VPS39 |
| 1638 | 200977_s_at | 3228.71 | 17.09 | 1.22 | 1.38 | AF090891 | Hs.5437 | TAX1BP1 |
| 1639 | 212899_at | 3232.68 | 17.19 | 1.21 | 1.37 | AB028951 | Hs.129836 | CDK11 |
| 1640 | 219229_at | 3233.91 | 17.20 | 1.20 | 1.35 | NM_013272 | Hs.113657 | SLCO3A1 |
| 1641 | 202589_at | 3234.41 | 17.21 | 1.18 | 1.31 | NM_001071 | Hs.87491 | TYMS |
| 1642 | 205584_at | 3235.00 | 17.22 | 1.22 | 1.38 | NM_024810 | Hs.169078 | CXorf45 |
| 1643 | AFFX- | 3235.28 | 17.22 | 1.15 | 1.26 | | | |
| | HUMGAPDH/M33197_5_at | | | | | | | |
| 1644 | 210639_s_at | 3237.77 | 17.27 | 1.20 | 1.35 | AF293841 | Hs.11171 | APG5L |
| 1645 | 204789_at | 3238.37 | 17.27 | 1.21 | 1.38 | NM_005892 | Hs.100217 | FMNL1 |
| 1646 | 219403_s_at | 3238.93 | 17.28 | 1.12 | 1.20 | NM_006665 | Hs.44227 | HPSE |
| 1647 | 201267_s_at | 3239.35 | 17.28 | 1.21 | 1.36 | AL545523 | Hs.250758 | PSMC3 |
| 1648 | 204131_s_at | 3240.82 | 17.31 | 1.20 | 1.34 | N25732 | Hs.14845 | FOXO3A |
| 1649 | 217432_s_at | 3241.24 | 17.31 | 1.23 | 1.41 | AF179281 | Hs.303154 | IDS |
| 1650 | 208862_s_at | 3241.36 | 17.30 | 1.21 | 1.37 | AW073672 | Hs.166011 | CTNND1 |
| 1651 | 208731_at | 3241.84 | 17.31 | 1.15 | 1.26 | AU158062 | Hs.78305 | RAB2 |
| 1652 | 201700_at | 3242.30 | 17.31 | 1.22 | 1.38 | NM_001760 | Hs.83173 | CCND3 |
| 1653 | 209894_at | 3243.29 | 17.31 | 1.10 | 1.17 | U50748 | Hs.23581 | LEPR |
| 1654 | 220326_s_at | 3245.61 | 17.36 | 1.22 | 1.38 | NM_018071 | Hs.22451 | FLJ10357 |
| 1655 | 220199_s_at | 3247.65 | 17.40 | 1.19 | 1.32 | NM_022831 | Hs.512696 | FLJ12806 |
| 1656 | 217766_s_at | 3249.09 | 17.44 | 1.23 | 1.40 | NM_014313 | Hs.107979 | SMP1 |
| 1657 | 212582_at | 3249.27 | 17.43 | 1.23 | 1.40 | AL049923 | Hs.109694 | OSBPL8 |
| 1658 | 212198_s_at | 3249.42 | 17.43 | 1.23 | 1.41 | AL515964 | Hs.79305 | TM9SF4 |
| 1659 | 202656_s_at | 3249.82 | 17.43 | 1.23 | 1.40 | BG107456 | Hs.77293 | SERTAD2 |
| 1660 | 217789_at | 3250.35 | 17.43 | 1.21 | 1.37 | NM_021249 | Hs.283443 | SNX6 |
| 1661 | 203233_at | 3250.91 | 17.43 | 1.20 | 1.35 | NM_000418 | Hs.75545 | IL4R |
| 1662 | 213726_x_at | 3252.97 | 17.47 | 1.19 | 1.33 | AA515698 | Hs.433615 | TUBB2 |
| 1663 | 204194_at | 3255.85 | 17.54 | 1.23 | 1.41 | NM_001186 | Hs.154276 | BACH1 |
| 1664 | 218505_at | 3257.54 | 17.58 | 1.21 | 1.37 | NM_024673 | Hs.413396 | FLJ12270 |
| 1665 | 212606_at | 3257.88 | 17.57 | 1.22 | 1.39 | AL536319 | Hs.105340 | WDFY3 |
| 1666 | 222221_x_at | 3259.61 | 17.61 | 1.21 | 1.37 | AY007161 | Hs.155119 | EHD1 |
| 1667 | 205316_at | 3261.24 | 17.65 | 1.21 | 1.36 | BF223679 | Hs.118747 | SLC15A2 |
| 1668 | 206723_s_at | 3261.62 | 17.65 | 1.18 | 1.31 | AF011466 | Hs.122575 | EDG4 |
| 1669 | 201037_at | 3265.84 | 17.75 | 1.17 | 1.30 | NM_002627 | Hs.26010 | PFKP |
| 1670 | 217865_at | 3266.43 | 17.75 | 1.22 | 1.38 | NM_018434 | Hs.155718 | RNF130 |
| 1671 | 202027_at | 3266.46 | 17.74 | 1.21 | 1.37 | NM_012264 | Hs.182626 | C22orf5 |
| 1672 | 202079_s_at | 3266.66 | 17.73 | 1.22 | 1.39 | AI633774 | Hs.457063 | OIP106 |
| 1673 | 200656_s_at | 3266.82 | 17.73 | 1.23 | 1.40 | NM_000918 | Hs.410578 | P4HB |
| 1674 | 219437_s_at | 3268.38 | 17.75 | 1.22 | 1.39 | NM_013275 | Hs.402727 | ANKRD11 |
| 1675 | 209199_s_at | 3269.08 | 17.76 | 1.19 | 1.33 | N22468 | Hs.368950 | MEF2C |
| 1676 | 221535_at | 3269.40 | 17.76 | 1.21 | 1.36 | AL136897 | Hs.436471 | FLJ11301 |
| 1677 | 211833_s_at | 3270.11 | 17.77 | 1.15 | 1.25 | U19599 | Hs.159428 | BAX |
| 1678 | 211632_at | 3273.37 | 17.84 | 1.21 | 1.37 | L34163 | Hs.383438 | — |
| 1679 | 208934_s_at | 3273.78 | 17.84 | 1.22 | 1.39 | AF342815 | Hs.4082 | LGALS8 |
| 1680 | 201071_x_at | 3273.80 | 17.83 | 1.22 | 1.39 | NM_012433 | Hs.334826 | SF3B1 |
| 1681 | 219361_s_at | 3273.84 | 17.82 | 1.23 | 1.40 | NM_022767 | Hs.436102 | FLJ12484 |
| 1682 | 202551_s_at | 3273.89 | 17.81 | 1.23 | 1.40 | BG546884 | Hs.170752 | CRIM1 |
| 1683 | 205010_at | 3276.17 | 17.85 | 1.17 | 1.30 | NM_019067 | Hs.438583 | FLJ10613 |
| 1684 | 217225_x_at | 3277.24 | 17.87 | 1.20 | 1.35 | AL512687 | Hs.227823 | NOMO2 /// |
| | | | | | | | | FLJ43542 |
| 1685 | 214289_at | 3277.45 | 17.86 | 1.22 | 1.38 | W86293 | Hs.352768 | PSMB1 |
| 1686 | 210580_x_at | 3278.01 | 17.87 | 1.21 | 1.37 | L25275 | — | SULT1A3 |
| 1687 | 206574_s_at | 3279.30 | 17.89 | 1.21 | 1.37 | NM_007079 | Hs.43666 | PTP4A3 |
| 1688 | 212993_at | 3280.53 | 17.92 | 1.21 | 1.37 | AA114166 | Hs.531457 | — |
| 1689 | 218552_at | 3281.33 | 17.92 | 1.18 | 1.31 | NM_018281 | Hs.170915 | FLJ10948 |
| 1690 | 220099_s_at | 3281.58 | 17.92 | 1.22 | 1.38 | NM_016007 | Hs.308777 | LUC7L2 |
| 1691 | 209760_at | 3282.57 | 17.93 | 1.21 | 1.38 | AL136932 | Hs.511944 | KIAA0922 |
| 1692 | 218149_s_at | 3285.58 | 17.99 | 1.22 | 1.39 | NM_017606 | Hs.27410 | ZNF395 |
| 1693 | 221059_s_at | 3289.31 | 18.08 | 1.20 | 1.35 | NM_021615 | Hs.289092 | COTL1 |
| 1694 | 205048_s_at | 3293.04 | 18.17 | 1.24 | 1.42 | NM_003832 | Hs.512656 | PSPH |
| 1695 | 203718_at | 3293.94 | 18.19 | 1.14 | 1.25 | NM_006702 | Hs.511760 | NTE |
| 1696 | 217734_s_at | 3294.86 | 18.20 | 1.21 | 1.37 | NM_018031 | Hs.8737 | WDR6 |
| 1697 | 205883_at | 3302.46 | 18.41 | 1.17 | 1.29 | NM_006006 | Hs.356349 | ZBTB16 |
| 1698 | 200052_s_at | 3303.24 | 18.42 | 1.18 | 1.32 | NM_004515 | Hs.75117 | ILF2 |
| 1699 | 209459_s_at | 3304.05 | 18.43 | 1.20 | 1.36 | AF237813 | Hs.1588 | ABAT |
| 1700 | 221764_at | 3304.65 | 18.43 | 1.20 | 1.34 | AL574186 | Hs.388956 | C19orf22 |
| 1701 | 214219_x_at | 3305.75 | 18.45 | 1.19 | 1.34 | BE646618 | Hs.95424 | MAP4K1 |
| 1702 | 203322_at | 3308.18 | 18.51 | 1.23 | 1.41 | AU145934 | Hs.533923 | KIAA0863 |
| 1703 | 203261_at | 3308.39 | 18.51 | 1.23 | 1.40 | NM_006571 | Hs.158427 | DCTN6 |
| 1704 | 218847_at | 3308.78 | 18.51 | 1.16 | 1.28 | NM_006548 | Hs.30299 | IMP-2 |
| 1705 | 212546_s_at | 3308.98 | 18.50 | 1.23 | 1.40 | AI126634 | Hs.169600 | KIAA0826 |
| 1706 | 202069_s_at | 3309.85 | 18.52 | 1.21 | 1.37 | AI826060 | Hs.250616 | IDH3A |
| 1707 | 201244_s_at | 3315.59 | 18.67 | 1.22 | 1.38 | NM_002880 | Hs.257266 | RAF1 |
| 1708 | 202151_s_at | 3315.91 | 18.66 | 1.20 | 1.35 | NM_016172 | Hs.9194 | UBADC1 |
| 1709 | 219256_s_at | 3316.48 | 18.67 | 1.19 | 1.33 | NM_018986 | Hs.61053 | SH3TC1 |
| 1710 | 208634_s_at | 3317.61 | 18.69 | 1.22 | 1.39 | AB029290 | Hs.372463 | MACF1 |
| 1711 | 202100_at | 3317.69 | 18.68 | 1.20 | 1.34 | BG169673 | Hs.348024 | RALB |
| 1712 | 200594_x_at | 3318.27 | 18.69 | 1.20 | 1.35 | NM_004501 | Hs.166463 | HNRPU |
| 1713 | 218848_at | 3318.46 | 18.68 | 1.23 | 1.40 | NM_024339 | Hs.412304 | MGC2655 |
| 1714 | 208828_at | 3319.87 | 18.71 | 1.23 | 1.40 | BC004170 | Hs.108112 | POLE3 |
| 1715 | 211656_x_at | 3320.66 | 18.72 | 1.18 | 1.31 | M32577 | Hs.409934 | HLA-DQB1 |
| 1716 | 204137_at | 3321.07 | 18.72 | 1.13 | 1.22 | NM_003272 | Hs.15791 | TM7SF1 |
| 1717 | 211596_s_at | 3321.58 | 18.72 | 1.11 | 1.19 | AB050468 | Hs.528353 | LRIG1 |
| 1718 | 220480_at | 3326.06 | 18.84 | 1.19 | 1.34 | NM_021973 | Hs.388245 | HAND2 |
| 1719 | 207075_at | 3327.71 | 18.87 | 1.19 | 1.33 | NM_004895 | Hs.159483 | CIAS1 |
| 1720 | 217264_s_at | 3327.78 | 18.86 | 1.21 | 1.36 | U81961 | Hs.446415 | SCNN1A |
| 1721 | 211323_s_at | 3327.98 | 18.85 | 1.22 | 1.39 | L38019 | Hs.149900 | ITPR1 |
| 1722 | 209106_at | 3329.30 | 18.87 | 1.22 | 1.38 | BF576458 | Hs.386092 | NCOA1 |
| 1723 | 221286_s_at | 3329.43 | 18.87 | 1.21 | 1.36 | NM_016459 | Hs.409563 | PACAP |
| 1724 | 215105_at | 3329.69 | 18.86 | 1.20 | 1.35 | U50531 | — | CG030 |
| 1725 | 221027_s_at | 3330.28 | 18.86 | 1.08 | 1.13 | NM_030821 | Hs.526406 | PLA2G12A |
| 1726 | 212030_at | 3332.04 | 18.90 | 1.20 | 1.36 | BG251218 | Hs.197184 | RBM25 |
| 1727 | 211100_x_at | 3333.21 | 18.92 | 1.21 | 1.36 | U82278 | — | LILRA2 |
| 1728 | 204055_s_at | 3334.73 | 18.95 | 1.18 | 1.32 | NM_005930 | Hs.434485 | CTAGE5 |
| 1729 | 220882_at | 3339.94 | 19.08 | 1.22 | 1.38 | NM_018612 | — | — |
| 1730 | 204254_s_at | 3341.87 | 19.11 | 1.16 | 1.28 | NM_000376 | Hs.2062 | VDR |
| 1731 | 213111_at | 3342.32 | 19.11 | 1.23 | 1.40 | AB023198 | Hs.158135 | PIP5K3 |
| 1732 | 208470_s_at | 3342.74 | 19.11 | 1.21 | 1.37 | NM_020995 | Hs.403931 | HP |
| 1733 | 214964_at | 3343.74 | 19.13 | 1.21 | 1.36 | AA554430 | Hs.487431 | — |
| 1734 | 203528_at | 3344.04 | 19.13 | 1.20 | 1.35 | NM_006378 | Hs.511748 | SEMA4D |
| 1735 | 201188_s_at | 3346.24 | 19.17 | 1.22 | 1.38 | D26351 | Hs.77515 | ITPR3 |
| 1736 | 210102_at | 3347.05 | 19.19 | 1.18 | 1.32 | BC001234 | Hs.152944 | LOH11CR2A |
| 1737 | 211771_s_at | 3347.56 | 19.20 | 1.16 | 1.28 | BC006101 | Hs.1101 | POU2F2 |
| 1738 | 214972_at | 3348.06 | 19.20 | 1.22 | 1.39 | AU144791 | Hs.5734 | MGEA5 |
| 1739 | 208073_x_at | 3349.84 | 19.24 | 1.22 | 1.39 | NM_003316 | Hs.132605 | TTC3 |
| 1740 | 216538_at | 3350.42 | 19.24 | 1.22 | 1.39 | AL049351 | Hs.302058 | — |
| 1741 | 215706_x_at | 3351.14 | 19.25 | 1.21 | 1.38 | BC002323 | Hs.75873 | ZYX |
| 1742 | 206587_at | 3351.26 | 19.24 | 1.21 | 1.38 | NM_006584 | Hs.73072 | CCT6B |
| 1743 | 219471_at | 3352.36 | 19.26 | 1.22 | 1.38 | NM_025113 | Hs.413071 | C13orf18 |
| 1744 | 210109_at | 3353.21 | 19.27 | 1.21 | 1.36 | AF191492 | — | NAG8 |
| 1745 | 218376_s_at | 3353.88 | 19.27 | 1.18 | 1.32 | NM_022765 | Hs.33476 | NICAL |
| 1746 | 211901_s_at | 3354.49 | 19.28 | 1.20 | 1.35 | AF073745 | Hs.89901 | PDE4A |
| 1747 | 210873_x_at | 3354.71 | 19.27 | 1.02 | 1.04 | U03891 | Hs.348983 | APOBEC3A |
| 1748 | 208739_x_at | 3355.93 | 19.30 | 1.22 | 1.39 | L76416 | Hs.380973 | SUMO2 |
| 1749 | 202625_at | 3357.49 | 19.33 | 1.17 | 1.29 | AI356412 | Hs.80887 | LYN |
| 1750 | 203633_at | 3357.65 | 19.32 | 1.19 | 1.33 | BF001714 | Hs.259785 | CPT1A |
| 1751 | 219788_at | 3358.09 | 19.33 | 1.20 | 1.35 | NM_013439 | Hs.122591 | PILRA |
| 1752 | 208836_at | 3359.00 | 19.34 | 1.17 | 1.30 | U51478 | Hs.76941 | ATP1B3 |
| 1753 | 213729_at | 3359.66 | 19.34 | 1.22 | 1.39 | Z78308 | Hs.298735 | FNBP3 |
| 1754 | 211890_x_at | 3359.98 | 19.34 | 1.21 | 1.37 | AF127765 | Hs.439343 | CAPN3 |
| 1755 | 216894_x_at | 3360.69 | 19.35 | 1.16 | 1.28 | D64137 | Hs.106070 | CDKN1C |
| 1756 | 211999_at | 3364.05 | 19.43 | 1.20 | 1.36 | Z48950 | Hs.180877 | H3F3B |
| 1757 | 221191_at | 3364.95 | 19.45 | 1.16 | 1.27 | NM_018991 | Hs.411818 | DKFZP434A0131 |
| 1758 | 221742_at | 3365.20 | 19.44 | 1.18 | 1.32 | BF037823 | Hs.321390 | CUGBP1 |
| 1759 | 203036_s_at | 3365.90 | 19.44 | 1.21 | 1.36 | AI027678 | Hs.77694 | MTSS1 |
| 1760 | 218295_s_at | 3366.47 | 19.44 | 1.20 | 1.35 | NM_007172 | Hs.362841 | NUP50 |
| 1761 | 212769_at | 3367.83 | 19.47 | 1.18 | 1.32 | AI567426 | Hs.287362 | TLE3 |
| 1762 | 202296_s_at | 3370.05 | 19.52 | 1.23 | 1.40 | NM_007033 | Hs.40500 | RER1 |
| 1763 | 207571_x_at | 3371.50 | 19.54 | 1.22 | 1.38 | NM_004848 | Hs.10649 | C1orf38 |
| 1764 | 206883_x_at | 3371.62 | 19.54 | 1.10 | 1.17 | NM_000174 | Hs.1144 | GP9 |
| 1765 | 208071_s_at | 3372.16 | 19.54 | 1.22 | 1.39 | NM_021708 | Hs.407964 | LAIR1 |
| 1766 | 212254_s_at | 3372.53 | 19.54 | 1.21 | 1.37 | AI798790 | Hs.443518 | DST |
| 1767 | 208654_s_at | 3373.47 | 19.55 | 1.16 | 1.27 | BF669455 | Hs.43910 | CD164 |
| 1768 | 206235_at | 3373.69 | 19.55 | 1.21 | 1.37 | NM_002312 | Hs.166091 | LIG4 |
| 1769 | 202406_s_at | 3375.70 | 19.61 | 1.22 | 1.39 | NM_003252 | Hs.335786 | TIAL1 |
| 1770 | 212678_at | 3375.92 | 19.60 | 1.22 | 1.38 | AW054826 | Hs.93207 | NF1 |
| 1771 | 202932_at | 3377.90 | 19.64 | 1.21 | 1.36 | NM_005433 | Hs.194148 | YES1 |
| 1772 | 214059_at | 3378.16 | 19.64 | 1.17 | 1.29 | BE049439 | Hs.82316 | IFI44 |
| 1773 | 222285_at | 3379.68 | 19.67 | 1.17 | 1.30 | AW134608 | Hs.439852 | IGHM |
| 1774 | 202480_s_at | 3380.84 | 19.70 | 1.21 | 1.37 | NM_004216 | Hs.169681 | DEDD |
| 1775 | 211746_x_at | 3380.91 | 19.69 | 1.22 | 1.39 | BC005932 | Hs.82159 | PSMA1 |
| 1776 | 208104_s_at | 3381.39 | 19.69 | 1.20 | 1.34 | NM_030935 | Hs.97717 | THG-1 |
| 1177 | 203062_s_at | 3381.84 | 19.69 | 1.21 | 1.37 | NM_014641 | Hs.433653 | MDC1 |
| 1778 | 216236_s_at | 3382.14 | 19.68 | 1.19 | 1.34 | AL110298 | Hs.401274 | SLC2A14 /// |
| | | | | | | | | SLC2A3 |
| 1779 | 217735_s_at | 3384.09 | 19.72 | 1.21 | 1.37 | AW007368 | Hs.434986 | HRI |
| 1780 | 218450_at | 3385.15 | 19.74 | 1.20 | 1.34 | NM_015987 | Hs.294133 | HEBP1 |
| 1781 | 213395_at | 3385.31 | 19.74 | 1.16 | 1.28 | AL022327 | Hs.74518 | MLC1 |
| 1782 | 221727_at | 3385.49 | 19.73 | 1.20 | 1.34 | AA456973 | Hs.229641 | PC4 |
| 1783 | 204251_s_at | 3385.70 | 19.73 | 1.18 | 1.32 | NM_014956 | Hs.18624 | Cep164 |
| 1784 | 34031_i_at | 3386.48 | 19.74 | 1.21 | 1.38 | U90269 | Hs.438833 | CCM1 |
| 1785 | 206174_s_at | 3389.69 | 19.82 | 1.18 | 1.31 | NM_002721 | Hs.356739 | PPP6C |
| 1786 | 211297_s_at | 3391.01 | 19.85 | 1.22 | 1.39 | L20320 | Hs.184298 | CDK7 |
| 1787 | 218389_s_at | 3391.27 | 19.84 | 1.20 | 1.35 | NM_016022 | Hs.108408 | APH-1A |
| 1788 | 208751_at | 3392.98 | 19.87 | 1.21 | 1.37 | BC001165 | Hs.75932 | NAPA |
| 1789 | 202352_s_at | 3394.94 | 19.91 | 1.21 | 1.36 | AI446530 | Hs.4295 | PSMD12 |
| 1790 | 201259_s_at | 3396.61 | 19.95 | 1.20 | 1.35 | AI768845 | Hs.80919 | SYPL |
| 1791 | 201573_s_at | 3396.84 | 19.95 | 1.18 | 1.32 | M75715 | Hs.77324 | ETF1 |
| 1792 | 221824_s_at | 3397.81 | 19.97 | 1.21 | 1.37 | AA770170 | Hs.288156 | MIR |
| 1793 | 200729_s_at | 3402.89 | 20.12 | 1.16 | 1.28 | NM_005722 | Hs.393201 | ACTR2 |
| 1794 | 213514_s_at | 3402.94 | 20.11 | 1.20 | 1.35 | AU158818 | Hs.432623 | DIAPH1 |
| 1795 | 203118_at | 3403.21 | 20.10 | 1.22 | 1.38 | NM_004716 | Hs.443752 | PCSK7 |
| 1796 | 201935_s_at | 3403.33 | 20.10 | 1.21 | 1.37 | AI768122 | Hs.402697 | EIF4G3 |
| 1797 | 210878_s_at | 3404.97 | 20.13 | 1.22 | 1.38 | BC001202 | Hs.24125 | JMJD1B |
| 1798 | 218420_s_at | 3405.44 | 20.13 | 1.22 | 1.38 | NM_025138 | Hs.318526 | C13orf23 |
| 1799 | 219322_s_at | 3406.51 | 20.15 | 1.22 | 1.39 | NM_017818 | Hs.31714 | WDR8 |
| 1800 | 59375_at | 3407.15 | 20.16 | 1.14 | 1.24 | AI825877 | Hs.390817 | MYO15B |
| 1801 | 202897_at | 3407.30 | 20.15 | 1.20 | 1.35 | AB023430 | Hs.156114 | PTPNS1 |
| 1802 | 216640_s_at | 3408.21 | 20.17 | 1.15 | 1.26 | AK026926 | Hs.212102 | TXNDC7 |
| 1803 | 207550_at | 3408.34 | 20.16 | 1.17 | 1.29 | NM_005373 | — | MPL |
| 1804 | 218676_s_at | 3408.61 | 20.16 | 1.22 | 1.39 | NM_021213 | Hs.285218 | PCTP |
| 1805 | 205467_at | 3410.76 | 20.20 | 1.20 | 1.34 | NM_001230 | Hs.5353 | CASP10 |
| 1806 | 205819_at | 3413.38 | 20.26 | 1.12 | 1.21 | NM_006770 | Hs.67726 | MARCO |
| 1807 | 212641_at | 3414.39 | 20.29 | 1.13 | 1.22 | AL023584 | Hs.75063 | HIVEP2 |
| 1808 | 205180_s_at | 3414.62 | 20.28 | 1.21 | 1.36 | NM_001109 | Hs.86947 | ADAM8 |
| 1809 | 210230_at | 3415.67 | 20.30 | 1.18 | 1.32 | BC003629 | — | — |
| 1810 | 218576_s_at | 3416.28 | 20.31 | 1.22 | 1.39 | NM_007240 | Hs.416216 | DUSP12 |
| 1811 | 221581_s_at | 3419.86 | 20.40 | 1.21 | 1.36 | AF257135 | Hs.56607 | WBSCR5 |
| 1812 | 204766_s_at | 3420.32 | 20.40 | 1.22 | 1.39 | NM_002452 | Hs.413078 | NUDT1 |
| 1813 | 213116_at | 3420.98 | 20.41 | 1.19 | 1.32 | AI191920 | Hs.2236 | NEK3 |
| 1814 | 201698_s_at | 3421.03 | 20.40 | 1.22 | 1.39 | NM_003769 | Hs.77608 | SFRS9 |
| 1815 | 211509_s_at | 3421.23 | 20.39 | 1.21 | 1.37 | AB015639 | Hs.436349 | RTN4 |
| 1816 | 201285_at | 3422.55 | 20.42 | 1.20 | 1.34 | NM_013446 | Hs.7838 | MKRN1 |
| 1817 | 202401_s_at | 3423.88 | 20.45 | 1.22 | 1.38 | NM_003131 | Hs.444086 | SRF |
| 1818 | 222035_s_at | 3425.81 | 20.49 | 1.17 | 1.30 | AI984479 | Hs.201085 | PAPOLA |
| 1819 | 218374_s_at | 3426.19 | 20.49 | 1.20 | 1.34 | NM_020374 | Hs.296198 | C12orf4 |
| 1820 | 214870_x_at | 3426.40 | 20.48 | 1.20 | 1.34 | AC002045 | Hs.449809 | LOC339047 /// |
| | | | | | | | | NPIP |
| 1821 | 212982_at | 3426.68 | 20.48 | 1.22 | 1.39 | AI621223 | Hs.4014 | ZDHHC17 |
| 1822 | 200980_s_at | 3427.71 | 20.50 | 1.22 | 1.39 | NM_000284 | Hs.1023 | PDHA1 |
| 1823 | 218660_at | 3428.65 | 20.51 | 1.16 | 1.27 | NM_003494 | Hs.1432 | DYSF |
| 1824 | 208517_x_at | 3430.02 | 20.54 | 1.20 | 1.35 | NM_001207 | Hs.446567 | BTF3 |
| 1825 | 209823_x_at | 3432.26 | 20.58 | 1.07 | 1.12 | M17955 | Hs.409934 | HLA-DQB1 |
| 1826 | 34206_at | 3432.47 | 20.58 | 1.16 | 1.28 | AB018325 | Hs.192190 | CENTD2 |
| 1827 | 203508_at | 3432.77 | 20.58 | 1.16 | 1.28 | NM_001066 | Hs.256278 | TNFRSF1B |
| 1828 | 212033_at | 3435.09 | 20.63 | 1.22 | 1.38 | BF055107 | Hs.197184 | RBM25 |
| 1829 | 205857_at | 3436.58 | 20.66 | 1.20 | 1.35 | AI269290 | Hs.50458 | SLC1BA2 |
| 1830 | 205885_s_at | 3436.87 | 20.66 | 1.17 | 1.30 | L12002 | Hs.528404 | ITGA4 |
| 1831 | 212655_at | 3437.39 | 20.66 | 1.19 | 1.33 | AB011151 | Hs.81505 | ZCCHC14 |
| 1832 | 201368_at | 3439.00 | 20.70 | 1.22 | 1.39 | U07802 | Hs.78909 | ZFP36L2 |
| 1833 | 202449_s_at | 3439.79 | 20.71 | 1.20 | 1.34 | NM_002957 | Hs.20084 | RXRA |
| 1834 | 212235_at | 3442.92 | 20.80 | 1.19 | 1.34 | AL575403 | Hs.301685 | PLXND1 |
| 1835 | 209105_at | 3445.42 | 20.86 | 1.20 | 1.34 | AI672428 | Hs.386092 | NCOA1 |
| 1836 | 200815_s_at | 3445.55 | 20.85 | 1.16 | 1.28 | L13386 | Hs.77318 | PAFAH1B1 |
| 1837 | 207384_at | 3445.60 | 20.84 | 1.21 | 1.37 | NM_005091 | Hs.137583 | PGLYRP1 |
| 1838 | 201841_s_at | 3445.96 | 20.85 | 1.18 | 1.31 | NM_001540 | Hs.76067 | HSPB1 |
| 1839 | 208056_s_at | 3445.97 | 20.84 | 1.21 | 1.37 | NM_005187 | Hs.110099 | CBFA2T3 |
| 1840 | 203378_at | 3447.35 | 20.87 | 1.23 | 1.40 | AB020631 | Hs.123654 | PCF11 |
| 1841 | 219515_at | 3447.68 | 20.86 | 1.23 | 1.41 | NM_020228 | Hs.275086 | PRDM10 |
| 1842 | 209824_s_at | 3448.45 | 20.87 | 1.23 | 1.41 | AB000812 | Hs.65734 | ARNTL |
| 1843 | 216952_s_at | 3449.73 | 20.90 | 1.21 | 1.36 | M94363 | Hs.76084 | LMNB2 |
| 1844 | 207819_s_at | 3449.83 | 20.89 | 1.16 | 1.28 | NM_000443 | Hs.73812 | ABCB4 |
| 1845 | 217659_at | 3449.97 | 20.88 | 1.21 | 1.36 | AA457019 | — | — |
| 1846 | 210120_s_at | 3450.30 | 20.88 | 1.22 | 1.38 | BC004349 | Hs.176657 | RANBP3 |
| 1847 | 218740_s_at | 3450.34 | 20.87 | 1.18 | 1.31 | NM_025197 | Hs.20157 | CDK5RAP3 |
| 1848 | 202760_s_at | 3450.42 | 20.86 | 1.17 | 1.30 | NM_007203 | — | AKAP2 |
| 1849 | 217938_s_at | 3451.62 | 20.89 | 1.21 | 1.36 | NM_020122 | Hs.5392 | KCMF1 |
| 1850 | 208002_s_at | 3451.86 | 20.89 | 1.17 | 1.30 | NM_007274 | Hs.435092 | BACH |
| 1851 | 217965_s_at | 3452.19 | 20.89 | 1.22 | 1.39 | NM_013260 | Hs.2729 | HCNP |
| 1852 | 200001_at | 3453.84 | 20.92 | 1.18 | 1.31 | NM_001749 | Hs.388469 | CAPNS1 |
| 1853 | 201246_s_at | 3454.16 | 20.92 | 1.21 | 1.36 | NM_017670 | Hs.473788 | OTUB1 |
| 1854 | 202512_s_at | 3454.43 | 20.92 | 1.22 | 1.38 | NM_004849 | Hs.11171 | APG5L |
| 1855 | 200863_s_at | 3455.28 | 20.93 | 1.15 | 1.26 | AI215102 | Hs.75618 | RAB11A |
| 1856 | 215659_at | 3458.14 | 21.00 | 1.18 | 1.31 | AK025174 | Hs.306777 | GSDML |
| 1857 | 200627_at | 3459.19 | 21.02 | 1.19 | 1.33 | BC003005 | Hs.355693 | TEBP |
| 1858 | 211275_s_at | 3460.51 | 21.04 | 1.15 | 1.26 | AF087942 | Hs.174071 | GYG |
| 1859 | 209653_at | 3461.27 | 21.05 | 1.18 | 1.31 | U93240 | Hs.288193 | KPNA4 |
| 1860 | 201760_s_at | 3461.90 | 21.05 | 1.20 | 1.36 | NM_018639 | Hs.459470 | WSB2 |
| 1861 | 219117_s_at | 3464.07 | 21.11 | 1.20 | 1.35 | NM_016594 | Hs.438695 | FKBP11 |
| 1862 | 213470_s_at | 3464.35 | 21.11 | 1.21 | 1.37 | BF983406 | Hs.202166 | HNRPH1 |
| 1863 | 202753_at | 3464.81 | 21.11 | 1.21 | 1.36 | NM_014814 | Hs.350939 | p44S10 |
| 1864 | 202405_at | 3465.09 | 21.11 | 1.21 | 1.36 | BF432532 | Hs.64840 | — |
| 1865 | 202833_s_at | 3471.40 | 21.30 | 1.15 | 1.26 | NM_000295 | Hs.297681 | SERPINA1 |
| 1866 | 217858_s_at | 3472.18 | 21.30 | 1.19 | 1.33 | NM_016607 | Hs.172788 | ARMCX3 |
| 1867 | 210895_s_at | 3472.37 | 21.30 | 1.18 | 1.32 | L25259 | Hs.27954 | CD86 |
| 1868 | 37965_at | 3473.02 | 21.30 | 1.21 | 1.37 | AA181053 | Hs.8836 | PARVB |
| 1869 | 202439_s_at | 3475.57 | 21.37 | 1.21 | 1.36 | NM_000202 | Hs.303154 | IDS |
| 1870 | 214039_s_at | 3476.68 | 21.39 | 1.11 | 1.19 | T15777 | Hs.296398 | LAPTM4B |
| 1871 | 218718_at | 3482.81 | 21.55 | 1.15 | 1.25 | NM_016205 | Hs.43080 | PDGFC |
| 1872 | 203492_x_at | 3483.78 | 21.56 | 1.22 | 1.38 | AA918224 | Hs.101014 | KIAA0092 |
| 1873 | 203938_s_at | 3485.06 | 21.59 | 1.20 | 1.34 | NM_005679 | Hs.153022 | TAF1C |
| 1874 | 216327_s_at | 3486.36 | 21.62 | 1.19 | 1.34 | AF287892 | Hs.447899 | SIGLEC8 |
| 1875 | 205801_s_at | 3489.25 | 21.69 | 1.18 | 1.32 | NM_015376 | Hs.24024 | RASGRP3 |
| 1876 | 218909_at | 3489.46 | 21.68 | 1.21 | 1.36 | NM_012424 | Hs.30352 | RPS6KC1 |
| 1877 | 210776_x_at | 3489.82 | 21.68 | 1.20 | 1.36 | M31222 | Hs.371282 | TCF3 |
| 1878 | 202780_at | 3492.17 | 21.74 | 1.21 | 1.36 | NM_000436 | Hs.177584 | OXCT1 |
| 1879 | 204064_at | 3494.61 | 21.79 | 1.20 | 1.36 | NM_005131 | Hs.1540 | THOC1 |
| 1880 | 200968_s_at | 3494.69 | 21.78 | 1.18 | 1.32 | NM_000942 | Hs.434937 | PPIB |
| 1881 | 213202_at | 3496.09 | 21.81 | 1.20 | 1.34 | N30342 | Hs.297483 | KIAA0339 |
| 1882 | 203207_s_at | 3499.37 | 21.90 | 1.21 | 1.37 | BF214329 | Hs.170198 | CHPPR |
| 1883 | 213397_x_at | 3500.10 | 21.91 | 1.18 | 1.30 | AI761728 | Hs.283749 | RNASE4 |
| 1884 | 211404_s_at | 3500.69 | 21.91 | 1.18 | 1.32 | BC004371 | Hs.279518 | APLP2 |
| 1885 | 219158_s_at | 3501.66 | 21.92 | 1.13 | 1.22 | NM_025085 | Hs.125034 | TBDN100 |
| 1886 | 207705_s_at | 3503.73 | 21.97 | 1.16 | 1.28 | NM_025176 | Hs.227743 | KIAA0980 |
| 1887 | 200668_s_at | 3505.27 | 22.01 | 1.21 | 1.37 | BC003395 | Hs.411826 | UBE2D3 |
| 1888 | 209184_s_at | 3505.48 | 22.01 | 1.19 | 1.33 | BF700086 | Hs.143648 | IRS2 |
| 1889 | 205546_s_at | 3506.79 | 22.04 | 1.19 | 1.33 | NM_003331 | Hs.75516 | TYK2 |
| 1890 | 204050_s_at | 3507.73 | 22.05 | 1.19 | 1.33 | NM_001833 | — | — |
| 1891 | 202167_s_at | 3508.22 | 22.05 | 1.21 | 1.37 | NM_022362 | Hs.288891 | MMS19L |
| 1892 | 210968_s_at | 3508.28 | 22.04 | 1.21 | 1.37 | AF333336 | Hs.436349 | RTN4 |
| 1893 | 37796_at | 3508.59 | 22.03 | 1.15 | 1.26 | AF053356 | Hs.125742 | LRCH4 |
| 1894 | 213618_at | 3508.76 | 22.02 | 1.21 | 1.37 | AB011152 | Hs.427719 | CENTD1 |
| 1895 | 212053_at | 3509.55 | 22.03 | 1.22 | 1.39 | AK025504 | Hs.439211 | KIAA0251 |
| 1896 | 214669_x_at | 3510.28 | 22.05 | 1.17 | 1.30 | BG485135 | Hs.377975 /// | IGKC |
| | | | | | | | Hs.449606 /// | |
| | | | | | | | Hs.494060 /// | |
| | | | | | | | Hs.512126 /// | |
| | | | | | | | Hs.534005 | |
| 1897 | 218822_s_at | 3510.35 | 22.03 | 1.15 | 1.25 | NM_024663 | Hs.378135 | NPEPL1 |
| 1898 | 212481_s_at | 3514.78 | 22.16 | 1.17 | 1.29 | AI214061 | Hs.250641 | TPM4 |
| 1899 | 200762_at | 3515.51 | 22.16 | 1.19 | 1.33 | NM_001386 | Hs.173381 | DPYSL2 |
| 1900 | 202112_at | 3516.13 | 22.17 | 1.15 | 1.26 | NM_000552 | Hs.440848 | VWF |
| 1901 | 217922_at | 3517.39 | 22.20 | 1.21 | 1.37 | AL157902 | Hs.367638 | MAN1A2 |
| 1902 | 201266_at | 3518.05 | 22.21 | 1.21 | 1.36 | NM_003330 | Hs.434367 | TXNRD1 |
| 1903 | 201878_at | 3518.97 | 22.22 | 1.21 | 1.37 | N25546 | Hs.181461 | ARIH1 |
| 1904 | 210129_s_at | 3519.31 | 22.23 | 1.19 | 1.33 | AF078842 | Hs.511982 | TTLL3 |
| 1905 | 217881_s_at | 3520.56 | 22.25 | 1.18 | 1.31 | NM_001256 | Hs.406631 | CDC27 |
| 1906 | 200638_s_at | 3521.86 | 22.29 | 1.19 | 1.34 | BC003623 | Hs.386834 | YWHAZ |
| 1907 | 210734_x_at | 3524.21 | 22.34 | 1.10 | 1.16 | M64240 | Hs.42712 | MAX |
| 1908 | 212470_at | 3524.45 | 22.33 | 1.20 | 1.35 | AB011088 | Hs.500367 | SPAG9 |
| 1909 | 215719_x_at | 3524.72 | 22.33 | 1.20 | 1.36 | X83493 | Hs.82359 | TNFRSF6 |
| 1910 | 208772_at | 3528.11 | 22.41 | 1.21 | 1.38 | AU160676 | — | MASK-BP3 |
| 1911 | 202124_s_at | 3528.22 | 22.40 | 1.20 | 1.34 | AV705253 | Hs.154248 | ALS2CR3 |
| 1912 | 204798_at | 3528.34 | 22.40 | 1.11 | 1.19 | NM_005375 | Hs.407830 | MYB |
| 1913 | 208696_at | 3528.71 | 22.41 | 1.20 | 1.35 | AF275798 | Hs.1600 | CCT5 |
| 1914 | 205463_s_at | 3529.06 | 22.40 | 1.08 | 1.13 | NM_002607 | Hs.376032 | PDGFA |
| 1915 | 213718_at | 3530.36 | 22.43 | 1.17 | 1.29 | BE222257 | Hs.211203 | RBM4 |
| 1916 | 218831_s_at | 3531.48 | 22.45 | 1.19 | 1.33 | NM_004107 | Hs.111903 | FCGRT |
| 1917 | 218137_s_at | 3532.16 | 22.46 | 1.21 | 1.37 | NM_021940 | Hs.410882 | SMAP1 |
| 1918 | 212690_at | 3533.80 | 22.50 | 1.22 | 1.38 | AB018268 | Hs.434966 | DDHD2 |
| 1919 | 209694_at | 3534.39 | 22.51 | 1.20 | 1.35 | M97655 | Hs.415877 | PTS |
| 1920 | 212101_at | 3535.44 | 22.53 | 1.19 | 1.33 | AU164321 | Hs.372741 | KPNA6 |
| 1921 | 209889_at | 3535.53 | 22.52 | 1.19 | 1.33 | AF274863 | Hs.18889 | SEC31L2 |
| 1922 | 204377_s_at | 3536.87 | 22.56 | 1.19 | 1.33 | NM_014703 | Hs.118738 | VprBP |
| 1923 | 208788_at | 3537.90 | 22.57 | 1.23 | 1.40 | AL136939 | Hs.343667 | ELOVL5 |
| 1924 | 218829_s_at | 3538.22 | 22.57 | 1.22 | 1.38 | NM_017780 | Hs.397426 | CHD7 |
| 1925 | 214405_at | 3538.24 | 22.56 | 1.18 | 1.31 | Z39557 | Hs.12533 | — |
| 1926 | 209268_at | 3540.39 | 22.62 | 1.21 | 1.36 | AF165513 | Hs.134231 | VPS45A |
| 1927 | 214027_x_at | 3540.65 | 22.61 | 1.19 | 1.33 | AA889653 | Hs.279604 | DES /// FAM48A |
| 1928 | 221136_at | 3542.23 | 22.64 | 1.20 | 1.34 | NM_016204 | Hs.279463 | GDF2 |
| 1929 | 205104_at | 3542.56 | 22.64 | 1.20 | 1.35 | NM_014723 | Hs.323833 | SNPH |
| 1930 | 208722_s_at | 3544.34 | 22.68 | 1.21 | 1.37 | BC001081 | Hs.7101 | ANAPC5 |
| 1931 | 202704_at | 3545.82 | 22.72 | 1.13 | 1.23 | AA675892 | Hs.178137 | TOB1 |
| 1932 | 203120_at | 3546.68 | 22.73 | 1.21 | 1.36 | NM_005426 | Hs.44585 | TP53BP2 |
| 1933 | 218953_s_at | 3547.09 | 22.73 | 1.18 | 1.31 | NM_024028 | Hs.257111 | MGC3265 |
| 1934 | 205530_at | 3547.17 | 22.73 | 1.20 | 1.35 | NM_004453 | Hs.511919 | ETFDH |
| 1935 | 221235_s_at | 3547.50 | 22.72 | 1.21 | 1.37 | NM_030825 | — | — |
| 1936 | 218178_s_at | 3547.98 | 22.72 | 1.20 | 1.35 | NM_020412 | — | CHMP1.5 |
| 1937 | 207127_s_at | 3548.12 | 22.72 | 1.20 | 1.36 | NM_021644 | Hs.156481 | HNRPH3 |
| 1938 | 220486_x_at | 3548.59 | 22.72 | 1.18 | 1.31 | NM_017698 | Hs.5472 | FLJ22679 |
| 1939 | 201986_at | 3550.45 | 22.76 | 1.22 | 1.39 | AB011165 | Hs.439144 | THRAP1 |
| 1940 | 221905_at | 3552.16 | 22.80 | 1.21 | 1.37 | BF516433 | Hs.386952 | CYLD |
| 1941 | 210684_s_at | 3553.12 | 22.82 | 1.19 | 1.32 | AF028825 | Hs.23731 | DLG4 |
| 1942 | 202546_at | 3553.85 | 22.83 | 1.17 | 1.30 | NM_003761 | — | — |
| 1943 | 209949_at | 3553.88 | 22.82 | 1.20 | 1.35 | BC001606 | Hs.949 | NCF2 |
| 1944 | 204088_at | 3555.35 | 22.85 | 1.21 | 1.36 | NM_002560 | Hs.321709 | P2RX4 |
| 1945 | 218974_at | 3555.54 | 22.85 | 1.19 | 1.33 | NM_018013 | Hs.346203 | FLJ10159 |
| 1946 | 203199_s_at | 3557.97 | 22.92 | 1.21 | 1.36 | N29717 | Hs.153792 | MTRR |
| 1947 | 218877_s_at | 3559.07 | 22.94 | 1.21 | 1.37 | NM_021820 | Hs.282575 | C6orf75 |
| 1948 | 205403_at | 3559.09 | 22.93 | 1.13 | 1.22 | NM_004633 | Hs.25333 | IL1R2 |
| 1949 | 205059_s_at | 3559.87 | 22.94 | 1.14 | 1.24 | NM_000203 | Hs.89560 | IDUA |
| 1950 | 212539_at | 3560.54 | 22.95 | 1.12 | 1.20 | AI422099 | Hs.429920 | CHD1L |
| 1951 | 201869_s_at | 3560.87 | 22.95 | 1.20 | 1.36 | BF593932 | Hs.76536 | TBL1X |
| 1952 | 218029_at | 3561.55 | 22.95 | 1.20 | 1.35 | NM_024519 | Hs.152717 | FLJ13725 |
| 1953 | 210178_x_at | 3562.30 | 22.96 | 1.20 | 1.34 | AF047448 | Hs.3530 | FUSIP1 |
| 1954 | 209174_s_at | 3563.19 | 22.98 | 1.21 | 1.37 | BC000978 | Hs.297389 | FLJ20259 |
| 1955 | 201236_s_at | 3565.58 | 23.03 | 1.18 | 1.32 | NM_006763 | Hs.75462 | BTG2 |
| 1956 | 205981_s_at | 3565.93 | 23.03 | 1.18 | 1.31 | NM_001564 | Hs.107153 | ING1L |
| 1957 | 217928_s_at | 3568.47 | 23.10 | 1.19 | 1.33 | NM_018312 | Hs.128218 | C11orf23 |
| 1958 | 213119_at | 3569.82 | 23.12 | 1.20 | 1.34 | AW058600 | Hs.409314 | SLC36A1 |
| 1959 | 205050_s_at | 3572.22 | 23.17 | 1.19 | 1.34 | NM_012324 | Hs.356523 | MAPK8IP2 |
| 1960 | 206789_s_at | 3573.09 | 23.18 | 1.20 | 1.35 | NM_002697 | Hs.444234 | POU2F1 |
| 1961 | 211716_x_at | 3573.18 | 23.17 | 1.19 | 1.33 | BC005851 | Hs.159161 | ARHGDIA |
| 1962 | 200059_s_at | 3573.94 | 23.18 | 1.20 | 1.35 | BC001360 | Hs.77273 | RHOA |
| 1963 | 212507_at | 3574.21 | 23.18 | 1.18 | 1.31 | D87446 | Hs.318783 | RW1 |
| 1964 | 202614_at | 3574.32 | 23.17 | 1.21 | 1.37 | NM_006345 | Hs.364615 | SLC30A9 |
| 1965 | 209815_at | 3577.46 | 23.26 | 1.11 | 1.18 | BG054916 | Hs.454253 | — |
| 1966 | 211289_x_at | 3578.08 | 23.27 | 1.20 | 1.35 | AF067524 | Hs.192316 | CDC2L1 |
| 1967 | 49111_at | 3578.69 | 23.28 | 1.21 | 1.36 | N80935 | Hs.12853 | — |
| 1968 | 201413_at | 3579.78 | 23.30 | 1.21 | 1.37 | NM_000414 | Hs.356894 | HSD17B4 |
| 1969 | 202541_at | 3581.00 | 23.33 | 1.19 | 1.33 | BF589679 | Hs.105656 | SCYE1 |
| 1970 | 208959_s_at | 3583.03 | 23.37 | 1.17 | 1.29 | BC005374 | Hs.154023 | TXNDC4 |
| 1971 | 201489_at | 3584.52 | 23.40 | 1.05 | 1.09 | BC005020 | Hs.381072 | PPIF |
| 1972 | 205878_at | 3586.53 | 23.44 | 1.21 | 1.37 | NM_002702 | Hs.2815 | POU6F1 |
| 1973 | 210092_at | 3587.93 | 23.47 | 1.18 | 1.31 | AF067173 | Hs.421576 | MAGOH |
| 1974 | 204313_s_at | 3588.66 | 23.48 | 1.22 | 1.38 | AA161486 | Hs.22315 | CREB1 |
| 1975 | 221631_at | 3590.45 | 23.52 | 1.20 | 1.35 | AB032946 | Hs.125116 | CACNA1I |
| 1976 | 209939_x_at | 3590.78 | 23.52 | 1.20 | 1.35 | AF005775 | Hs.355724 | CFLAR |
| 1977 | 217933_s_at | 3592.16 | 23.55 | 1.05 | 1.08 | NM_015907 | Hs.182579 | LAP3 |
| 1978 | 221160_s_at | 3592.67 | 23.55 | 1.12 | 1.21 | NM_019855 | Hs.117694 | CABP3 /// CABP5 |
| 1979 | 209522_s_at | 3593.72 | 23.57 | 1.17 | 1.29 | BC000723 | Hs.12068 | CRAT |
| 1980 | 217952_x_at | 3594.77 | 23.59 | 1.21 | 1.37 | BF430956 | Hs.348921 | PHF3 |
| 1981 | 214753_at | 3596.05 | 23.62 | 1.21 | 1.36 | AW084068 | Hs.131727 | PFAAP5 |
| 1982 | 205508_at | 3596.61 | 23.63 | 1.15 | 1.26 | NM_001037 | Hs.408883 | SCN1B |
| 1983 | 219646_at | 3598.83 | 23.69 | 1.21 | 1.38 | NM_017702 | Hs.62771 | FLJ20186 |
| 1984 | 209341_s_at | 3599.07 | 23.68 | 1.21 | 1.37 | AU153366 | Hs.413513 | IKBKB |
| 1985 | 215415_s_at | 3599.15 | 23.68 | 1.21 | 1.37 | U70064 | Hs.130188 | CHS1 |
| 1986 | 206108_s_at | 3599.84 | 23.69 | 1.16 | 1.27 | NM_006275 | Hs.6891 | SFRS6 |
| 1987 | 209314_s_at | 3600.08 | 23.68 | 1.17 | 1.30 | AK024258 | Hs.221040 | HBS1L |
| 1988 | 204182_s_at | 3600.58 | 23.69 | 1.14 | 1.25 | NM_014007 | Hs.355581 | ZNF297B |
| 1989 | 207842_s_at | 3601.95 | 23.72 | 1.21 | 1.37 | NM_007359 | Hs.350229 | CASC3 |
| 1990 | 207628_s_at | 3602.03 | 23.71 | 1.19 | 1.33 | NM_017528 | Hs.413036 | WBSCR22 |
| 1991 | 203865_s_at | 3603.14 | 23.73 | 1.21 | 1.38 | NM_015833 | Hs.148822 | ADARB1 |
| 1992 | 214172_x_at | 3604.99 | 23.78 | 1.21 | 1.36 | BG032035 | Hs.285346 | RYK |
| 1993 | 217772_s_at | 3605.02 | 23.77 | 1.20 | 1.35 | NM_014342 | Hs.279609 | MTCH2 |
| 1994 | 205976_at | 3608.61 | 23.87 | 1.21 | 1.36 | NM_014929 | Hs.84429 | KIAA0971 |
| 1995 | 218093_s_at | 3609.02 | 23.86 | 1.20 | 1.34 | NM_017664 | Hs.164969 | ANKRD10 |
| 1996 | 203155_at | 3610.45 | 23.89 | 1.20 | 1.35 | NM_012432 | Hs.345058 | SETDB1 |
| 1997 | 210469_at | 3611.61 | 23.91 | 1.19 | 1.33 | BC002915 | Hs.500245 | DLG5 |
| 1998 | 212692_s_at | 3614.19 | 23.98 | 1.21 | 1.36 | W60686 | Hs.209846 | LRBA |
| 1999 | 203959_s_at | 3615.35 | 24.00 | 1.20 | 1.34 | NM_014870 | Hs.528723 | KIAA0478 |
| 2000 | 219649_at | 3615.50 | 24.00 | 1.21 | 1.37 | NM_013339 | Hs.80042 | ALG6 |
| 2001 | 220104_at | 3616.28 | 24.00 | 1.16 | 1.28 | NM_020119 | Hs.35254 | ZC3HAV1 |
| 2002 | 210252_s_at | 3616.94 | 24.01 | 1.20 | 1.34 | AB002356 | Hs.82548 | MADD |
| 2003 | 216289_at | 3616.97 | 24.00 | 1.18 | 1.32 | AU148039 | Hs.454099 | GPR144 |
| 2004 | 202000_at | 3617.60 | 24.01 | 1.16 | 1.28 | BC002772 | Hs.274416 | NDUFA6 |
| 2005 | 201294_s_at | 3618.59 | 24.02 | 1.03 | 1.05 | N24643 | Hs.315379 | WSB1 |
| 2006 | 203948_s_at | 3618.79 | 24.02 | 1.18 | 1.32 | J02694 | — | — |
| 2007 | 222054_at | 3619.21 | 24.02 | 1.19 | 1.32 | BF511556 | Hs.232058 | — |
| 2008 | 214130_s_at | 3619.83 | 24.03 | 1.20 | 1.35 | AI821791 | Hs.502577 | PDE4DIP |
| 2009 | 207686_s_at | 3619.89 | 24.02 | 1.22 | 1.38 | NM_001228 | Hs.243491 | CASP8 |
| 2010 | 215722_s_at | 3621.33 | 24.06 | 1.20 | 1.35 | AJ130971 | Hs.434901 | SNRPA1 |
| 2011 | 219235_s_at | 3623.06 | 24.10 | 1.20 | 1.35 | NM_023923 | Hs.225641 | PHACTR4 |
| 2012 | 200723_s_at | 3623.34 | 24.10 | 1.15 | 1.25 | NM_005898 | Hs.278672 | M11S1 |
| 2013 | 200779_at | 3624.69 | 24.13 | 1.19 | 1.33 | NM_001675 | Hs.181243 | ATF4 |
| 2014 | 203966_s_at | 3624.80 | 24.12 | 1.17 | 1.30 | NM_021003 | Hs.130036 | PPM1A |
| 2015 | 214188_at | 3625.08 | 24.11 | 1.17 | 1.30 | AW665096 | — | — |
| 2016 | 202176_at | 3625.21 | 24.11 | 1.22 | 1.38 | NM_000122 | Hs.77929 | ERCC3 |
| 2017 | 212017_at | 3625.99 | 24.12 | 1.21 | 1.36 | BF677404 | — | LOC130074 |
| 2018 | 219278_at | 3626.08 | 24.11 | 1.17 | 1.29 | NM_004672 | Hs.194694 | MAP3K6 |
| 2019 | 221602_s_at | 3627.59 | 24.14 | 1.20 | 1.35 | AF057557 | Hs.58831 | TOSO |
| 2020 | 206114_at | 3627.66 | 24.13 | 1.14 | 1.24 | NM_004438 | Hs.244624 | EPHA4 |
| 2021 | 209034_at | 3627.93 | 24.13 | 1.19 | 1.34 | AF279899 | Hs.75969 | PNRC1 |
| 2022 | 208977_x_at | 3632.71 | 24.27 | 1.17 | 1.29 | BC004188 | Hs.433615 | TUBB2 |
| 2023 | 218520_at | 3633.46 | 24.28 | 1.19 | 1.33 | NM_013254 | Hs.432466 | TBK1 |
| 2024 | 205789_at | 3633.56 | 24.27 | 1.18 | 1.31 | NM_001766 | Hs.1799 | CD1D |
| 2025 | 202174_s_at | 3635.46 | 24.32 | 1.21 | 1.37 | NM_006197 | Hs.348501 | PCM1 |
| 2026 | 218217_at | 3635.90 | 24.32 | 1.18 | 1.31 | NM_021626 | Hs.431107 | SCPEP1 |
| 2027 | 202033_s_at | 3636.12 | 24.32 | 1.17 | 1.29 | BG402105 | Hs.151202 | RB1CC1 |
| 2028 | 207106_s_at | 3637.62 | 24.36 | 1.18 | 1.31 | NM_002344 | Hs.434481 | LTK |
| 2029 | 211994_at | 3638.16 | 24.36 | 1.21 | 1.36 | AI742553 | Hs.275999 | PRKWNK1 |
| 2030 | 208820_at | 3638.24 | 24.35 | 1.16 | 1.27 | AL037339 | Hs.434281 | PTK2 |
| 2031 | 216207_x_at | 3638.25 | 24.34 | 1.14 | 1.23 | AW408194 | Hs.390427 | IGKV1D-13 |
| 2032 | 216950_s_at | 3640.60 | 24.40 | 1.04 | 1.06 | X14355 | Hs.77424 | FCGR1A |
| 2033 | 219980_at | 3641.48 | 24.42 | 1.17 | 1.29 | NM_025097 | Hs.407155 | FLJ21106 |
| 2034 | 201164_s_at | 3642.04 | 24.42 | 1.21 | 1.37 | BG474429 | Hs.153834 | PUM1 |
| 2035 | 204490_s_at | 3642.82 | 24.44 | 1.18 | 1.32 | M24915 | Hs.306278 | CD44 |
| 2036 | 204506_at | 3643.52 | 24.44 | 1.16 | 1.28 | AL544951 | Hs.280604 | PPP3R1 |
| 2037 | 214781_at | 3644.39 | 24.46 | 1.19 | 1.34 | AL162013 | — | — |
| 2038 | 201670_s_at | 3644.40 | 24.45 | 1.05 | 1.09 | M68956 | Hs.318603 | MARCKS |
| 2039 | 206214_at | 3645.14 | 24.46 | 1.01 | 1.02 | NM_005084 | Hs.93304 | PLA2G7 |
| 2040 | 214918_at | 3645.90 | 24.47 | 1.16 | 1.27 | AK024911 | Hs.385766 | HNRPM |
| 2041 | 214373_at | 3646.53 | 24.48 | 1.10 | 1.18 | AI582773 | Hs.443268 | — |
| 2042 | 201636_at | 3647.86 | 24.52 | 1.19 | 1.34 | BG025078 | Hs.408096 | FXR1 |
| 2043 | 203041_s_at | 3649.19 | 24.54 | 1.16 | 1.28 | J04183 | Hs.232432 | LAMP2 |
| 2044 | 214833_at | 3650.91 | 24.59 | 1.21 | 1.37 | AB007958 | Hs.119387 | KIAA0792 |
| 2045 | 206707_x_at | 3650.94 | 24.57 | 1.19 | 1.34 | NM_015864 | Hs.389488 | C6orf32 |
| 2046 | 208921_s_at | 3651.51 | 24.58 | 1.13 | 1.23 | L12387 | Hs.422340 | SRI |
| 2047 | 204425_at | 3651.60 | 24.57 | 1.17 | 1.30 | NM_001666 | Hs.3109 | ARHGAP4 |
| 2048 | 208778_s_at | 3651.99 | 24.57 | 1.21 | 1.37 | BC000665 | Hs.4112 | TCP1 |
| 2049 | 203115_at | 3652.44 | 24.57 | 1.18 | 1.30 | AU152635 | Hs.443610 | FECH |
| 2050 | 218277_s_at | 3652.44 | 24.56 | 1.19 | 1.34 | NM_024612 | — | — |
| 2051 | 210997_at | 3653.11 | 24.57 | 1.20 | 1.35 | M77227 | Hs.396530 | HGF |
| 2052 | 218682_s_at | 3654.30 | 24.59 | 1.20 | 1.36 | NM_018158 | Hs.306000 | SLC4A1AP |
| 2053 | 207180_s_at | 3655.57 | 24.61 | 1.18 | 1.32 | NM_006410 | Hs.90753 | HTATIP2 |
| 2054 | 207511_s_at | 3658.99 | 24.70 | 1.21 | 1.36 | NM_015680 | Hs.4973 | C2orf24 |
| 2055 | 38398_at | 3660.23 | 24.72 | 1.19 | 1.33 | AB002356 | Hs.82548 | MADD |
| 2056 | 201038_s_at | 3661.25 | 24.74 | 1.19 | 1.34 | T67821 | Hs.356089 | ANP32A |
| 2057 | 214880_x_at | 3663.12 | 24.79 | 1.20 | 1.34 | D90453 | Hs.443811 | CALD1 |
| 2058 | 212980_at | 3663.21 | 24.78 | 1.18 | 1.31 | AL050376 | Hs.122440 | AHSA2 |
| 2059 | 213318_s_at | 3664.33 | 24.80 | 1.20 | 1.35 | BG028844 | Hs.440900 | BAT3 |
| 2060 | 218947_s_at | 3664.36 | 24.79 | 1.15 | 1.27 | NM_018109 | Hs.173946 | PAPD1 |
| 2061 | 213301_x_at | 3665.23 | 24.80 | 1.19 | 1.34 | AL538264 | Hs.183858 | TIF1 |
| 2062 | 221482_s_at | 3669.30 | 24.91 | 1.14 | 1.24 | BC003418 | Hs.7351 | ARPP-19 |
| 2063 | 202254_at | 3670.73 | 24.94 | 1.21 | 1.37 | AB007900 | Hs.375008 | — |
| 2064 | 215299_x_at | 3671.72 | 24.97 | 1.18 | 1.32 | U37025 | Hs.142 | SULT1A1 |
| 2065 | 203645_s_at | 3671.79 | 24.96 | 1.19 | 1.34 | NM_004244 | Hs.74076 | CD163 |
| 2066 | 37577_at | 3673.24 | 24.99 | 1.20 | 1.36 | U79256 | Hs.80305 | ARHGAP19 |
| 2067 | 210715_s_at | 3674.11 | 25.00 | 1.18 | 1.31 | AF027205 | Hs.31439 | SPINT2 |
| 2068 | 203163_at | 3675.81 | 25.04 | 1.21 | 1.37 | NM_005886 | Hs.275675 | KATNB1 |
| 2069 | 208881_x_at | 3676.54 | 25.05 | 1.20 | 1.35 | BC005247 | Hs.76038 | IDI1 |
| 2070 | 211953_s_at | 3677.21 | 25.06 | 1.20 | 1.34 | AU148466 | Hs.113503 | RANBP5 |
| 2071 | 201533_at | 3677.68 | 25.07 | 1.11 | 1.19 | NM_001904 | Hs.410086 | CTNNB1 |
| 2072 | 91703_at | 3678.03 | 25.06 | 1.16 | 1.28 | AA149545 | Hs.438942 | DKFZp762C186 |
| 2073 | 211286_x_at | 3678.89 | 25.08 | 1.19 | 1.32 | L29349 | Hs.520937 | CSF2RA |
| 2074 | 210609_s_at | 3679.54 | 25.09 | 1.16 | 1.27 | BC000474 | Hs.50649 | TP53I3 |
| 2075 | 215031_x_at | 3679.92 | 25.09 | 1.20 | 1.35 | BG420893 | Hs.69554 | RNF126 |
| 2076 | 218803_at | 3680.92 | 25.11 | 1.17 | 1.30 | NM_018223 | Hs.23794 | CHFR |
| 2077 | 211662_s_at | 3681.27 | 25.11 | 1.19 | 1.33 | L08666 | Hs.355927 | VDAC2 |
| 2078 | 208694_at | 3681.74 | 25.11 | 1.19 | 1.33 | U47077 | Hs.415749 | PRKDC |
| 2079 | 212882_at | 3682.26 | 25.11 | 1.20 | 1.35 | AB018338 | Hs.22926 | KLHL18 |
| 2080 | 204062_s_at | 3683.36 | 25.12 | 1.19 | 1.34 | BG526973 | Hs.168762 | ULK2 |
| 2081 | 202147_s_at | 3683.56 | 25.11 | 1.11 | 1.19 | NM_001550 | Hs.7879 | IFRD1 |
| 2082 | 212523_s_at | 3683.77 | 25.11 | 1.19 | 1.33 | D63480 | Hs.381058 | KIAA0146 |
| 2083 | 219770_at | 3684.08 | 25.11 | 1.20 | 1.34 | NM_024659 | Hs.159460 | GTDC1 |
| 2084 | 221050_s_at | 3684.18 | 25.10 | 1.18 | 1.32 | NM_019096 | Hs.13011 | GTPBP2 |
| 2085 | 200713_s_at | 3684.46 | 25.10 | 1.19 | 1.32 | NM_012325 | Hs.408754 | MAPRE1 |
| 2086 | 201444_s_at | 3686.02 | 25.13 | 1.17 | 1.29 | NM_005785 | Hs.183434 | ATP6AP2 |
| 2087 | 215329_s_at | 3686.53 | 25.14 | 1.19 | 1.33 | AL031282 | Hs.192316 | CDC2L1 |
| 2088 | 336_at | 3687.62 | 25.16 | 1.10 | 1.17 | D38081 | Hs.442530 | TBXA2R |
| 2089 | 200750_s_at | 3687.71 | 25.15 | 1.19 | 1.32 | AF054183 | Hs.10842 | RAN |
| 2090 | 219624_at | 3688.24 | 25.15 | 1.17 | 1.30 | NM_004874 | Hs.194726 | BAG4 |
| 2091 | 202722_s_at | 3689.34 | 25.18 | 1.18 | 1.31 | NM_002056 | Hs.1674 | GFPT1 |
| 2092 | 214311_at | 3691.53 | 25.23 | 1.19 | 1.33 | AI767884 | Hs.155165 | ZFPL1 |
| 2093 | 219484_at | 3691.71 | 25.22 | 1.20 | 1.35 | NM_013320 | Hs.55601 | HCFC2 |
| 2094 | 216977_x_at | 3692.52 | 25.23 | 1.21 | 1.36 | AJ130972 | Hs.434901 | SNRPA1 |
| 2095 | 203704_s_at | 3692.97 | 25.23 | 1.11 | 1.19 | AW118862 | Hs.171942 | RREB1 |
| 2096 | 219099_at | 3692.98 | 25.22 | 1.13 | 1.21 | NM_020375 | Hs.24792 | C12orf5 |
| 2097 | 207606_s_at | 3694.42 | 25.26 | 1.21 | 1.38 | NM_018287 | Hs.396643 | ARHGAP12 |
| 2098 | 212299_at | 3695.67 | 25.28 | 1.21 | 1.37 | AL117502 | Hs.7200 | NEK9 |
| 2099 | 213581_at | 3695.86 | 25.27 | 1.19 | 1.33 | BF446180 | Hs.367900 | PDCD2 |
| 2100 | 205671_s_at | 3695.92 | 25.26 | 1.16 | 1.27 | NM_002120 | Hs.1802 | HLA-DOB |
| 2101 | 201898_s_at | 3696.98 | 25.28 | 1.18 | 1.31 | AI126625 | Hs.379466 | UBE2A |
| 2102 | 220586_at | 3698.85 | 25.33 | 1.20 | 1.35 | NM_025134 | Hs.119053 | CHD9 |
| 2103 | 207668_x_at | 3699.38 | 25.33 | 1.16 | 1.28 | NM_005742 | Hs.212102 | TXNDC7 |
| 2104 | 211090_s_at | 3700.27 | 25.35 | 1.09 | 1.16 | Z25435 | Hs.198891 | PRPF4B |
| 2105 | 213887_s_at | 3700.64 | 25.34 | 1.16 | 1.28 | AI554759 | Hs.24301 | POLR2E |
| 2106 | 214946_x_at | 3702.05 | 25.37 | 1.18 | 1.32 | AV728658 | Hs.375174 | LOC387680 |
| 2107 | 209864_at | 3702.05 | 25.36 | 1.11 | 1.19 | AB045118 | Hs.140720 | FRAT2 |
| 2108 | 204294_at | 3704.28 | 25.41 | 1.19 | 1.32 | NM_000481 | Hs.102 | AMT |
| 2109 | 216180_s_at | 3706.41 | 25.46 | 1.18 | 1.31 | AK026758 | Hs.434494 | SYNJ2 |
| 2110 | 207287_at | 3707.43 | 25.48 | 1.17 | 1.29 | NM_025026 | — | FLJ14107 |
| 2111 | 201914_s_at | 3707.48 | 25.47 | 1.16 | 1.28 | AK001465 | Hs.330767 | SEC63 |
| 2112 | 211458_s_at | 3708.22 | 25.48 | 1.16 | 1.28 | AF180519 | Hs.336429 | GABARAPL1 |
| 2113 | 211780_x_at | 3709.12 | 25.50 | 1.18 | 1.32 | BC006163 | Hs.74617 | DCTN1 |
| 2114 | 201356_at | 3710.15 | 25.52 | 1.21 | 1.36 | BF129339 | Hs.406277 | SF3A1 |
| 2115 | 213329_at | 3710.83 | 25.53 | 1.19 | 1.33 | AA742261 | Hs.5003 | FNBP2 |
| 2116 | 214464_at | 3713.76 | 25.61 | 1.14 | 1.23 | NM_003607 | Hs.18586 | CDC42BPA |
| 2117 | 202820_at | 3714.51 | 25.62 | 1.19 | 1.33 | NM_001621 | Hs.170087 | AHR |
| 2118 | 218223_s_at | 3714.77 | 25.61 | 1.12 | 1.21 | NM_016274 | Hs.173380 | CKIP-1 |
| 2119 | 212309_at | 3716.17 | 25.64 | 1.20 | 1.34 | AV725315 | Hs.108614 | CLASP2 |
| 2120 | 209585_s_at | 3717.53 | 25.66 | 1.16 | 1.28 | AF084943 | Hs.95907 | MINPP1 |
| 2121 | 221761_at | 3717.72 | 25.66 | 1.20 | 1.35 | AA628948 | Hs.90011 | ADSS |
| 2122 | 204232_at | 3717.74 | 25.64 | 1.17 | 1.29 | NM_004106 | Hs.433300 | FCER1G |
| 2123 | 200748_s_at | 3719.42 | 25.69 | 1.17 | 1.29 | NM_002032 | Hs.446345 | FTH1 |
| 2124 | 218289_s_at | 3720.32 | 25.71 | 1.21 | 1.37 | NM_024818 | Hs.170737 | UBE1DC1 |
| 2125 | 212267_at | 3722.15 | 25.76 | 1.20 | 1.35 | D87450 | Hs.439188 | KIAA0261 |
| 2126 | 212375_at | 3723.34 | 25.78 | 1.19 | 1.34 | AL563727 | Hs.306094 | EP400 |
| 2127 | 216260_at | 3724.40 | 25.80 | 1.14 | 1.24 | AK001827 | Hs.87889 | DICER1 |
| 2128 | 219062_s_at | 3726.04 | 25.84 | 1.17 | 1.29 | NM_017742 | Hs.114191 | ZCCHC2 |
| 2129 | 218503_at | 3727.12 | 25.86 | 1.20 | 1.34 | NM_017794 | Hs.257696 | KIAA1797 |
| 2130 | 219040_at | 3727.75 | 25.87 | 1.17 | 1.29 | NM_024535 | Hs.7258 | CRN7 |
| 2131 | 211871_x_at | 3727.81 | 25.86 | 1.19 | 1.34 | AF300650 | Hs.155090 | GNB5 |
| 2132 | 203504_s_at | 3728.03 | 25.86 | 1.20 | 1.34 | NM_005502 | Hs.147259 | ABCA1 |
| 2133 | 204401_at | 3728.29 | 25.85 | 1.17 | 1.29 | NM_002250 | Hs.10082 | KCNN4 |
| 2134 | 214924_s_at | 3728.60 | 25.85 | 1.19 | 1.33 | AK000754 | Hs.457063 | OIP106 |
| 2135 | 200669_s_at | 3728.76 | 25.84 | 1.18 | 1.32 | NM_003340 | Hs.411826 | UBE2D3 |
| 2136 | 200953_s_at | 3728.98 | 25.84 | 1.20 | 1.34 | NM_001759 | Hs.376071 | CCND2 |
| 2137 | 203749_s_at | 3730.27 | 25.86 | 1.18 | 1.31 | AI806984 | Hs.361071 | RARA |
| 2138 | 218815_s_at | 3732.91 | 25.93 | 1.17 | 1.29 | NM_018022 | Hs.30925 | FLJ10199 |
| 2139 | 204385_at | 3732.95 | 25.92 | 1.19 | 1.33 | NM_003937 | Hs.444471 | KYNU |
| 2140 | 218989_x_at | 3734.05 | 25.95 | 1.20 | 1.35 | NM_022902 | Hs.438748 | SLC30A5 |
| 2141 | 203313_s_at | 3734.30 | 25.94 | 1.21 | 1.37 | NM_003244 | Hs.161999 | TGIF |
| 2142 | 202119_s_at | 3735.22 | 25.96 | 1.21 | 1.36 | NM_003909 | Hs.14158 | CPNE3 |
| 2143 | 214894_x_at | 3735.57 | 25.96 | 1.19 | 1.33 | AK023285 | Hs.372463 | MACF1 |
| 2144 | 211675_s_at | 3736.34 | 25.97 | 1.19 | 1.33 | AF054589 | Hs.132739 | HIC |
| 2145 | 218673_s_at | 3737.34 | 26.00 | 1.17 | 1.29 | NM_006395 | Hs.278607 | APG7L |
| 2146 | 219378_at | 3737.71 | 25.99 | 1.17 | 1.30 | NM_024561 | Hs.13277 | FLJ22054 |
| 2147 | 203818_s_at | 3739.30 | 26.03 | 1.19 | 1.32 | NM_006802 | — | — |
| 2148 | 212820_at | 3741.10 | 26.08 | 1.16 | 1.27 | AB020663 | Hs.200828 | RC3 |
| 2149 | 212901_s_at | 3741.91 | 26.09 | 1.08 | 1.14 | BF732638 | Hs.21992 | CSTF2T |
| 2150 | 200852_x_at | 3744.94 | 26.17 | 1.17 | 1.29 | NM_005273 | Hs.185172 | GNB2 |
| 2151 | 202730_s_at | 3747.14 | 26.24 | 1.20 | 1.34 | NM_014456 | Hs.257697 | PDCD4 |
| 2152 | 203825_at | 3748.05 | 26.25 | 1.17 | 1.29 | NM_007371 | Hs.86896 | BRD3 |
| 2153 | 201060_x_at | 3748.60 | 26.25 | 1.16 | 1.27 | AI537887 | Hs.439776 | STOM |
| 2154 | 212751_at | 3749.55 | 26.26 | 1.20 | 1.34 | BG290646 | Hs.458359 | UBE2N |
| 2155 | 219233_s_at | 3750.57 | 26.28 | 1.17 | 1.29 | NM_018530 | Hs.306777 | GSDML |
| 2156 | 203397_s_at | 3751.25 | 26.29 | 1.19 | 1.33 | BF063271 | Hs.278611 | GALNT3 |
| 2157 | 211395_x_at | 3753.77 | 26.35 | 1.20 | 1.34 | U90940 | Hs.126384 | FCGR2A /// |
| | | | | | | | | FCGR2B /// |
| | | | | | | | | FCGR2C |
| 2158 | 200696_s_at | 3753.83 | 26.34 | 1.16 | 1.28 | NM_000177 | Hs.446537 | GSN |
| 2159 | 216997_x_at | 3754.56 | 26.35 | 1.17 | 1.29 | AL358975 | Hs.494269 | TLE4 |
| 2160 | 200066_at | 3755.23 | 26.36 | 1.20 | 1.35 | AF182645 | Hs.421245 | IK |
| 2161 | 201570_at | 3758.22 | 26.45 | 1.18 | 1.31 | NM_015380 | Hs.505824 | CGI-51 |
| 2162 | 208620_at | 3758.71 | 26.45 | 1.16 | 1.27 | U24223 | Hs.2853 | PCBP1 |
| 2163 | 200778_s_at | 3761.20 | 26.53 | 1.16 | 1.28 | AI191427 | Hs.131736 | NEDD5 |
| 2164 | 212638_s_at | 3761.45 | 26.53 | 1.19 | 1.33 | BF131791 | Hs.450060 | WWP1 |
| 2165 | 201676_x_at | 3762.12 | 26.54 | 1.20 | 1.35 | NM_002786 | Hs.82159 | PSMA1 |
| 2166 | 200744_s_at | 3762.18 | 26.53 | 1.17 | 1.29 | AI741124 | Hs.304694 | GNB1 |
| 2167 | 208774_at | 3762.64 | 26.53 | 1.20 | 1.34 | AV700224 | Hs.528476 | — |
| 2168 | 215378_at | 3763.63 | 26.56 | 1.18 | 1.31 | AU148255 | Hs.406840 | EIF4EBP3 |
| 2169 | 212086_x_at | 3765.06 | 26.59 | 1.18 | 1.31 | AK026584 | Hs.436441 | LMNA |
| 2170 | 220051_at | 3765.58 | 26.60 | 1.17 | 1.29 | NM_006799 | Hs.72026 | PRSS21 |
| 2171 | 211742_s_at | 3766.41 | 26.61 | 1.20 | 1.34 | BC005926 | Hs.5509 | EVI2B |
| 2172 | 203276_at | 3766.46 | 26.60 | 1.06 | 1.10 | NM_005573 | Hs.89497 | LMNB1 |
| 2173 | 212538_at | 3767.97 | 26.64 | 1.15 | 1.25 | AL576253 | Hs.362177 | DOCK9 |
| 2174 | 200755_s_at | 3769.11 | 26.66 | 1.19 | 1.33 | BF939365 | Hs.7753 | CALU |
| 2175 | 213446_s_at | 3771.02 | 26.71 | 1.02 | 1.04 | AI679073 | — | — |
| 2176 | 215427_s_at | 3772.08 | 26.74 | 1.19 | 1.32 | AL117532 | Hs.81505 | ZCCHC14 |
| 2177 | 217947_at | 3772.42 | 26.74 | 1.18 | 1.32 | NM_017801 | Hs.380627 | CKLFSF6 |
| 2178 | 209430_at | 3773.82 | 26.77 | 1.19 | 1.33 | AJ001017 | — | — |
| 2179 | 200097_s_at | 3774.63 | 26.78 | 1.20 | 1.34 | AI701949 | Hs.307544 | HNRPK |
| 2180 | 209069_s_at | 3775.20 | 26.79 | 1.19 | 1.33 | BC001124 | Hs.180877 | H3F3B |
| 2181 | 214494_s_at | 3775.48 | 26.78 | 1.16 | 1.27 | NM_005200 | Hs.311765 | SPG7 |
| 2182 | 212929_s_at | 3776.51 | 26.80 | 1.17 | 1.29 | W68158 | Hs.375174 | FLJ10824 /// |
| | | | | | | | | LOC387680 |
| 2183 | 205051_s_at | 3776.51 | 26.79 | 1.18 | 1.32 | NM_000222 | Hs.81665 | KIT |
| 2184 | 213475_s_at | 3777.10 | 26.80 | 1.15 | 1.25 | AC002310 | Hs.174103 | ITGAL |
| 2185 | 201555_at | 3778.77 | 26.85 | 1.20 | 1.34 | NM_002388 | Hs.179565 | MCM3 |
| 2186 | 204547_at | 3779.10 | 26.85 | 1.15 | 1.26 | NM_006822 | Hs.302498 | RAB40B |
| 2187 | 201946_s_at | 3779.16 | 26.84 | 1.14 | 1.24 | AL545982 | Hs.189772 | CCT2 |
| 2188 | 208660_at | 3780.03 | 26.85 | 1.20 | 1.36 | BC000105 | Hs.430606 | CS |
| 2189 | 212314_at | 3781.33 | 26.88 | 1.15 | 1.25 | AB018289 | Hs.49500 | KIAA0746 |
| 2190 | 201346_at | 3781.56 | 26.88 | 1.19 | 1.33 | NM_024551 | Hs.334854 | ADIPOR2 |
| 2191 | 216903_s_at | 3782.11 | 26.89 | 1.17 | 1.30 | AK022697 | Hs.61628 | CBARA1 |
| 2192 | 219190_s_at | 3782.50 | 26.89 | 1.15 | 1.26 | NM_017629 | Hs.437864 | EIF2C4 |
| 2193 | 202422_s_at | 3783.08 | 26.90 | 1.18 | 1.31 | NM_022977 | Hs.269559 | ACSL4 |
| 2194 | 205312_at | 3783.42 | 26.89 | 1.19 | 1.34 | NM_003120 | Hs.157441 | SPI1 |
| 2195 | 203891_s_at | 3783.69 | 26.89 | 1.14 | 1.23 | NM_001348 | Hs.153908 | DAPK3 |
| 2196 | 213078_x_at | 3786.57 | 26.97 | 1.19 | 1.33 | AI889513 | Hs.352614 | LOC254531 |
| 2197 | 201540_at | 3786.79 | 26.97 | 1.11 | 1.19 | NM_001449 | Hs.421383 | FHL1 |
| 2198 | 209338_at | 3788.04 | 27.00 | 1.18 | 1.32 | U03494 | Hs.154970 | TFCP2 |
| 2199 | 204202_at | 3788.19 | 26.99 | 1.19 | 1.33 | NM_017604 | Hs.446063 | IQCE |
| 2200 | 201818_at | 3789.78 | 27.02 | 1.14 | 1.23 | NM_024830 | Hs.179882 | FLJ12443 |
| 2201 | 207809_s_at | 3791.94 | 27.08 | 1.19 | 1.33 | NM_001183 | Hs.6551 | ATP6AP1 |
| 2202 | 203079_s_at | 3792.08 | 27.07 | 1.20 | 1.34 | NM_003591 | Hs.82919 | CUL2 |
| 2203 | 212370_x_at | 3793.38 | 27.10 | 1.18 | 1.32 | AL080183 | Hs.449662 | FLJ10824 |
| 2204 | 200820_at | 3796.98 | 27.21 | 1.12 | 1.21 | NM_002812 | Hs.78466 | PSMD8 |
| 2205 | 213251_at | 3797.68 | 27.22 | 1.19 | 1.33 | AV712064 | Hs.135705 | SMARCA5 |
| 2206 | 202071_at | 3799.21 | 27.26 | 1.11 | 1.19 | NM_002999 | Hs.252189 | SDC4 |
| 2207 | 218224_at | 3800.11 | 27.27 | 1.16 | 1.27 | NM_006029 | Hs.194709 | PNMA1 |
| 2208 | 217518_at | 3801.34 | 27.29 | 1.11 | 1.19 | BF056029 | Hs.362731 | FER1L3 |
| 2209 | 200916_at | 3802.00 | 27.31 | 1.16 | 1.27 | NM_003564 | Hs.406504 | TAGLN2 |
| 2210 | 208456_s_at | 3804.26 | 27.36 | 1.18 | 1.31 | NM_012250 | Hs.206097 | RRAS2 |
| 2211 | 217945_at | 3804.54 | 27.36 | 1.20 | 1.35 | NM_025238 | Hs.321501 | BTBD1 |
| 2212 | 204538_x_at | 3804.84 | 27.36 | 1.17 | 1.30 | NM_006985 | Hs.449809 | LOC339047 /// |
| | | | | | | | | NPIP |
| 2213 | 210257_x_at | 3805.05 | 27.36 | 1.18 | 1.30 | AF212995 | Hs.155976 | CUL4B |
| 2214 | 218380_at | 3807.09 | 27.42 | 1.16 | 1.27 | NM_021730 | Hs.104305 | NALP1 |
| 2215 | 217900_at | 3807.13 | 27.41 | 1.20 | 1.34 | NM_018060 | Hs.262823 | FLJ10326 |
| 2216 | 35776_at | 3807.28 | 27.40 | 1.17 | 1.30 | AF064243 | Hs.66392 | ITSN1 |
| 2217 | 202667_s_at | 3807.48 | 27.39 | 1.19 | 1.32 | NM_006979 | Hs.278721 | SLC39A7 |
| 2218 | 201975_at | 3807.92 | 27.39 | 1.19 | 1.33 | NM_002956 | Hs.410113 | RSN |
| 2219 | 201611_s_at | 3807.92 | 27.38 | 1.17 | 1.29 | NM_012405 | Hs.183212 | ICMT |
| 2220 | 206176_at | 3808.93 | 27.40 | 1.10 | 1.17 | NM_001718 | Hs.285671 | BMP6 |
| 2221 | 213437_at | 3809.04 | 27.39 | 1.15 | 1.26 | AA861784 | Hs.7972 | RIPX |
| 2222 | 39582_at | 3809.45 | 27.39 | 1.20 | 1.35 | AL050166 | Hs.386952 | CYLD |
| 2223 | 215454_x_at | 3810.63 | 27.42 | 1.18 | 1.31 | AI831055 | Hs.1074 | SFTPC |
| 2224 | 210146_x_at | 3811.53 | 27.43 | 1.06 | 1.10 | AF004231 | Hs.306230 | LILRB2 |
| 2225 | 202157_s_at | 3811.60 | 27.42 | 1.19 | 1.33 | U69546 | Hs.211610 | CUGBP2 |
| 2226 | 203688_at | 3812.35 | 27.44 | 1.19 | 1.34 | NM_000297 | Hs.458291 | PKD2 |
| 2227 | 201101_s_at | 3812.96 | 27.45 | 0.96 | 0.94 | BE963370 | Hs.80338 | BCLAF1 |
| 2228 | 201920_at | 3814.45 | 27.49 | 1.13 | 1.23 | NM_005415 | Hs.110855 | SLC20A1 |
| 2229 | 202688_at | 3814.85 | 27.49 | 1.01 | 1.01 | NM_003810 | Hs.387871 | TNFSF10 |
| 2230 | 203254_s_at | 3815.23 | 27.49 | 1.18 | 1.32 | NM_006289 | Hs.375001 | TLN1 |
| 2231 | 208717_at | 3816.56 | 27.51 | 1.20 | 1.35 | BC001669 | Hs.151134 | OXA1L |
| 2232 | 219342_at | 3818.00 | 27.55 | 1.19 | 1.33 | NM_022900 | Hs.324725 | CAS1 |
| 2233 | 205961_s_at | 3819.24 | 27.59 | 1.19 | 1.33 | NM_004682 | Hs.351305 | PSIP1 |
| 2234 | 210845_s_at | 3819.61 | 27.59 | 1.11 | 1.18 | U08839 | Hs.179657 | PLAUR |
| 2235 | 202697_at | 3821.17 | 27.63 | 1.19 | 1.33 | NM_007006 | Hs.446393 | CPSF5 |
| 2236 | 202775_s_at | 3821.39 | 27.63 | 1.18 | 1.31 | NM_004592 | Hs.213739 | SFRS8 |
| 2237 | 214681_at | 3823.14 | 27.67 | 1.15 | 1.26 | AI830490 | Hs.1466 | GK |
| 2238 | 201336_at | 3823.25 | 27.66 | 1.19 | 1.33 | BC003570 | Hs.66708 | VAMP3 |
| 2239 | 211356_x_at | 3823.47 | 27.66 | 1.11 | 1.19 | U66495 | Hs.23581 | LEPR |
| 2240 | 208308_s_at | 3824.08 | 27.67 | 1.16 | 1.28 | NM_000175 | Hs.406458 | GPI |
| 2241 | 202511_s_at | 3825.88 | 27.71 | 1.18 | 1.31 | AK001899 | Hs.11171 | APG5L |
| 2242 | 203669_s_at | 3827.12 | 27.74 | 1.19 | 1.33 | NM_012079 | Hs.288627 | DGAT1 |
| 2243 | 206075_s_at | 3828.04 | 27.76 | 1.19 | 1.33 | NM_001895 | Hs.446484 | CSNK2A1 |
| 2244 | 219024_at | 3828.22 | 27.75 | 1.20 | 1.35 | NM_021622 | Hs.287830 | PLEKHA1 |
| 2245 | 201011_at | 3831.03 | 27.84 | 1.17 | 1.29 | NM_002950 | Hs.2280 | RPN1 |
| 2246 | 212509_s_at | 3831.22 | 27.83 | 0.99 | 0.99 | BF968134 | Hs.250723 | FLJ46603 |
| 2247 | 214657_s_at | 3832.00 | 27.85 | 1.16 | 1.28 | AU134977 | Hs.240443 | — |
| 2248 | 205398_s_at | 3832.67 | 27.86 | 1.19 | 1.33 | NM_005902 | Hs.288261 | SMAD3 |
| 2249 | 219151_s_at | 3832.98 | 27.86 | 1.19 | 1.33 | NM_007081 | Hs.355874 | RABL2A /// |
| | | | | | | | | RABL2B |
| 2250 | 218539_at | 3835.17 | 27.92 | 1.21 | 1.36 | NM_017943 | Hs.15467 | FBXO34 |
| 2251 | 213313_at | 3836.44 | 27.94 | 1.19 | 1.34 | AI922519 | Hs.55099 | GPR21 |
| 2252 | 205116_at | 3836.52 | 27.93 | 1.18 | 1.32 | NM_000426 | Hs.445120 | LAMA2 |
| 2253 | 215262_at | 3837.06 | 27.93 | 1.13 | 1.22 | AF052160 | Hs.142570 | — |
| 2254 | 216041_x_at | 3837.33 | 27.93 | 1.17 | 1.29 | AK023348 | — | — |
| 2255 | 215016_x_at | 3837.35 | 27.92 | 1.18 | 1.31 | BC004912 | Hs.443518 | DST |
| 2256 | 212478_at | 3837.67 | 27.92 | 1.14 | 1.23 | H65865 | Hs.75277 | FLJ13910 |
| 2257 | 209467_s_at | 3837.88 | 27.92 | 1.18 | 1.30 | BC002755 | Hs.79516 | MKNK1 |
| 2258 | 221290_s_at | 3839.64 | 27.97 | 1.18 | 1.30 | NM_016473 | Hs.454758 | MUM1 |
| 2259 | 204781_s_at | 3842.36 | 28.05 | 1.15 | 1.25 | NM_000043 | Hs.82359 | TNFRSF6 |
| 2260 | 64432_at | 3842.54 | 28.04 | 1.20 | 1.34 | W05463 | Hs.333120 | FLJ39616 |
| 2261 | 209318_x_at | 3843.05 | 28.05 | 1.09 | 1.16 | BG547855 | Hs.132911 | PLAGL1 |
| 2262 | 204199_at | 3845.52 | 28.11 | 1.19 | 1.33 | NM_014636 | Hs.432842 | RALGPS1 |
| 2263 | 209439_s_at | 3847.44 | 28.16 | 1.19 | 1.33 | D38616 | Hs.54941 | PHKA2 |
| 2264 | 214703_s_at | 3847.66 | 28.16 | 1.19 | 1.34 | AW954107 | Hs.200661 | MAN2B2 |
| 2265 | 202623_at | 3848.46 | 28.17 | 1.13 | 1.23 | NM_018453 | Hs.433269 | C14orf11 |
| 2266 | 200947_s_at | 3849.13 | 28.19 | 1.20 | 1.34 | NM_005271 | Hs.355697 | GLUD1 |
| 2267 | 212613_at | 3849.29 | 28.18 | 1.13 | 1.23 | AI991252 | Hs.376046 | BTN3A2 |
| 2268 | 202822_at | 3851.10 | 28.22 | 1.18 | 1.32 | BF221852 | Hs.180398 | LPP |
| 2269 | 216253_s_at | 3851.26 | 28.22 | 1.19 | 1.33 | N73272 | Hs.8836 | PARVB |
| 2270 | 213902_at | 3852.25 | 28.24 | 1.14 | 1.25 | AI379338 | Hs.324808 | ASAH1 |
| 2271 | 203112_s_at | 3856.13 | 28.36 | 1.18 | 1.31 | NM_005663 | Hs.21771 | WHSC2 |
| 2272 | 202792_s_at | 3856.37 | 28.36 | 1.16 | 1.27 | NM_014678 | Hs.449098 | KIAA0685 |
| 2273 | 219507_at | 3856.71 | 28.36 | 1.01 | 1.01 | NM_016625 | Hs.191381 | MGC12197 |
| 2274 | 201174_s_at | 3857.31 | 28.37 | 1.18 | 1.31 | NM_018975 | Hs.274428 | TERF2IP |
| 2275 | 216766_at | 3857.37 | 28.36 | 1.18 | 1.31 | AK025152 | — | — |
| 2276 | 214831_at | 3857.61 | 28.36 | 1.21 | 1.36 | AK024944 | Hs.33032 | — |
| 2277 | 203689_s_at | 3857.68 | 28.35 | 1.20 | 1.35 | AI743037 | Hs.89764 | FMR1 |
| 2278 | 204245_s_at | 3859.42 | 28.39 | 1.20 | 1.34 | AW242755 | Hs.446320 | RPP14 |
| 2279 | 64408_s_at | 3860.32 | 28.41 | 1.17 | 1.30 | AW025529 | Hs.528715 | CALML4 |
| 2280 | 208992_s_at | 3861.59 | 28.45 | 1.16 | 1.28 | BC000627 | Hs.421342 | STAT3 |
| 2281 | 203552_at | 3861.91 | 28.45 | 1.17 | 1.30 | AW298170 | Hs.246970 | MAP4K5 |
| 2282 | 203723_at | 3862.21 | 28.45 | 1.20 | 1.35 | NM_002221 | Hs.78877 | ITPKB |
| 2283 | 209222_s_at | 3862.34 | 28.43 | 1.17 | 1.30 | BC000296 | Hs.15519 | OSBPL2 |
| 2284 | 207194_s_at | 3884.35 | 28.49 | 1.14 | 1.24 | NM_001544 | Hs.512159 | ICAM4 |
| 2285 | 222133_s_at | 3565.05 | 28.50 | 1.18 | 1.31 | AK022280 | Hs.44159 | CGI-72 |
| 2286 | 204053_x_at | 3868.81 | 28.62 | 1.18 | 1.31 | U96180 | Hs.253309 | PTEN |
| 2287 | 202684_s_at | 3870.48 | 28.67 | 1.17 | 1.30 | AB020966 | Hs.8086 | RNMT |
| 2288 | 212657_s_at | 3871.25 | 28.68 | 1.06 | 1.09 | U65590 | Hs.81134 | IL1RN |
| 2289 | 215300_s_at | 3872.47 | 28.71 | 1.17 | 1.30 | AK022172 | Hs.396595 | FMO5 |
| 2290 | 216976_s_at | 3872.63 | 28.71 | 1.19 | 1.34 | X96588 | Hs.285346 | RYK |
| 2291 | 201022_s_at | 3874.32 | 28.75 | 1.17 | 1.29 | NM_006870 | Hs.408576 | DSTN |
| 2292 | 219136_s_at | 3874.45 | 28.74 | 1.19 | 1.33 | NM_022773 | Hs.58362 | FLJ12681 |
| 2293 | 201569_s_at | 3874.49 | 28.73 | 1.16 | 1.27 | NM_015380 | Hs.505824 | CGI-51 |
| 2294 | 219820_at | 3874.57 | 28.72 | 1.18 | 1.31 | NM_014037 | — | — |
| 2295 | 220560_at | 3875.31 | 28.74 | 1.11 | 1.19 | NM_014144 | Hs.272100 | C11orf21 |
| 2296 | 200765_x_at | 3875.93 | 28.74 | 1.17 | 1.29 | NM_001903 | Hs.254321 | CTNNA1 |
| 2297 | 210410_s_at | 3876.11 | 28.74 | 1.18 | 1.31 | AF034759 | Hs.132399 | MSH5 |
| 2298 | 208729_x_at | 3876.46 | 28.74 | 1.15 | 1.26 | D83043 | Hs.77961 | HLA-B |
| 2299 | 219470_x_at | 3878.23 | 28.79 | 1.17 | 1.30 | NM_019084 | Hs.281348 | CCNJ |
| 2300 | 208961_s_at | 3878.27 | 28.78 | 1.12 | 1.21 | AB017493 | Hs.285313 | COPEB |
| 2301 | 208523_x_at | 3879.24 | 28.80 | 1.11 | 1.18 | NM_003525 | — | HIST1H2BI |
| 2302 | 202518_at | 3880.14 | 28.82 | 1.20 | 1.34 | NM_001707 | Hs.408219 | BCL7B |
| 2303 | 220035_at | 3880.28 | 28.81 | 1.15 | 1.26 | NM_024923 | Hs.292119 | NUP210 |
| 2304 | 215330_at | 3882.56 | 28.88 | 1.15 | 1.26 | AL049991 | Hs.226103 | — |
| 2305 | 202675_at | 3884.31 | 28.93 | 1.17 | 1.29 | NM_003000 | Hs.64 | SDHB |
| 2306 | 208636_at | 3885.35 | 28.95 | 1.11 | 1.20 | AI082078 | Hs.119000 | ACTN1 |
| 2307 | 214281_s_at | 3885.61 | 28.95 | 1.13 | 1.22 | AA524525 | Hs.48297 | RCHY1 |
| 2308 | 220740_s_at | 3886.05 | 28.95 | 1.18 | 1.32 | NM_005135 | Hs.4876 | SLC12A6 |
| 2309 | 202388_at | 3887.06 | 28.97 | 1.16 | 1.27 | NM_002923 | Hs.78944 | RGS2 |
| 2310 | 204083_s_at | 3889.34 | 29.03 | 1.16 | 1.27 | NM_003289 | Hs.300772 | TPM2 |
| 2311 | 208420_x_at | 3889.58 | 29.03 | 1.17 | 1.30 | NM_003170 | — | — |
| 2312 | 217787_s_at | 3889.70 | 29.02 | 1.19 | 1.34 | AL525086 | Hs.307582 | GALNT2 |
| 2313 | 205896_at | 3890.22 | 29.02 | 1.16 | 1.28 | NM_003059 | Hs.441130 | SLC22A4 |
| 2314 | 200640_at | 3890.32 | 29.01 | 1.19 | 1.32 | NM_003406 | Hs.386834 | YWHAZ |
| 2315 | 215555_at | 3892.80 | 29.08 | 1.13 | 1.22 | AU158442 | — | — |
| 2316 | 208999_at | 3894.73 | 29.13 | 1.17 | 1.29 | D86957 | Hs.307944 | SEPT8 |
| 2317 | 213808_at | 3897.11 | 29.20 | 1.16 | 1.28 | BE674466 | Hs.432317 | ADAM23 |
| 2318 | 218497_s_at | 3897.87 | 29.21 | 1.18 | 1.31 | NM_002936 | Hs.511960 | RNASEH1 |
| 2319 | 202423_at | 3898.13 | 29.21 | 1.20 | 1.34 | NM_006766 | Hs.93231 | MYST3 |
| 2320 | 205068_s_at | 3898.64 | 29.21 | 1.08 | 1.13 | BE671084 | Hs.132942 | ARHGAP26 |
| 2321 | 209311_at | 3899.29 | 29.22 | 1.18 | 1.31 | D87461 | Hs.410026 | BCL2L2 |
| 2322 | 202467_s_at | 3899.37 | 29.21 | 1.16 | 1.28 | NM_004236 | Hs.30212 | TRIP15 |
| 2323 | 212315_s_at | 3899.93 | 29.22 | 1.19 | 1.33 | AA502912 | Hs.292119 | NUP210 |
| 2324 | 200691_s_at | 3902.58 | 29.29 | 1.19 | 1.34 | BC000478 | Hs.184233 | HSPA9B |
| 2325 | 217906_at | 3903.52 | 29.32 | 1.20 | 1.35 | NM_014315 | Hs.415236 | KLHDC2 |
| 2326 | 212569_at | 3903.57 | 29.31 | 1.19 | 1.32 | AV699744 | Hs.8118 | KIAA0650 |
| 2327 | 206825_at | 3906.46 | 29.39 | 1.06 | 1.10 | NM_000916 | Hs.2820 | OXTR |
| 2328 | 203857_s_at | 3907.27 | 29.40 | 1.17 | 1.30 | NM_006810 | Hs.76901 | PDIR |
| 2329 | 201646_at | 3907.51 | 29.40 | 1.17 | 1.29 | AA885297 | Hs.349656 | SCARB2 |
| 2330 | 221878_at | 3907.82 | 29.39 | 1.20 | 1.34 | BF110411 | Hs.370214 | — |
| 2331 | 221743_at | 3908.41 | 29.40 | 1.19 | 1.32 | AI472139 | Hs.321390 | CUGBP1 |
| 2332 | 208072_s_at | 3908.70 | 29.40 | 1.17 | 1.29 | NM_003648 | Hs.115907 | DGKD |
| 2333 | 212912_at | 3910.98 | 29.46 | 1.17 | 1.30 | AI992251 | Hs.301664 | RPS6KA2 |
| 2334 | 217143_s_at | 3913.99 | 29.55 | 0.99 | 0.98 | X06557 | Hs.2014 | TRDD3 /// TRD@ |
| 2335 | 201783_s_at | 3915.63 | 29.59 | 1.17 | 1.29 | NM_021975 | Hs.132594 | RELA |
| 2336 | 201014_s_at | 3919.81 | 29.72 | 1.18 | 1.30 | NM_006452 | Hs.444439 | PAICS |
| 2337 | 220243_at | 3920.03 | 29.72 | 1.18 | 1.32 | NM_014155 | Hs.178499 | HSPC063 |
| 2338 | 201191_at | 3920.24 | 29.72 | 1.20 | 1.35 | H15647 | Hs.433429 | PITPNA |
| 2339 | 204206_at | 3920.58 | 29.72 | 1.20 | 1.34 | NM_020310 | Hs.253552 | MNT |
| 2340 | 221547_at | 3922.94 | 29.79 | 1.17 | 1.29 | BC000794 | Hs.306456 | PRPF18 |
| 2341 | 204834_at | 3923.04 | 29.78 | 1.15 | 1.26 | NM_006682 | Hs.351808 | FGL2 |
| 2342 | 204249_s_at | 3923.85 | 29.80 | 1.17 | 1.29 | NM_005574 | Hs.283063 | LMO2 |
| 2343 | 207005_s_at | 3924.63 | 29.82 | 1.16 | 1.27 | NM_000657 | Hs.79241 | BCL2 |
| 2344 | 210011_s_at | 3928.07 | 29.92 | 1.19 | 1.33 | BC000527 | Hs.374477 | EWSR1 |
| 2345 | 211339_s_at | 3928.07 | 29.91 | 1.14 | 1.24 | D13720 | — | — |
| 2346 | 214437_s_at | 3928.91 | 29.93 | 1.18 | 1.30 | NM_005412 | Hs.75069 | SHMT2 |
| 2347 | 201880_at | 3929.15 | 29.92 | 1.19 | 1.33 | AL040708 | Hs.181461 | ARIH1 |
| 2348 | 204838_s_at | 3930.79 | 29.97 | 0.92 | 0.88 | NM_014381 | Hs.279843 | MLH3 |
| 2349 | 204115_at | 3930.93 | 29.96 | 1.00 | 1.00 | NM_004126 | Hs.83381 | GNG11 |
| 2350 | 212137_at | 3931.45 | 29.97 | 1.19 | 1.33 | AV746402 | Hs.6214 | LARP |
| 2351 | 205516_x_at | 3932.24 | 29.98 | 1.18 | 1.32 | NM_012127 | Hs.23476 | CIZ1 |
| 2352 | 202221_s_at | 3932.59 | 29.98 | 1.10 | 1.18 | AV727101 | Hs.271730 | EP300 |
| 2353 | 220143_x_at | 3933.41 | 30.00 | 1.18 | 1.31 | NM_018032 | Hs.16803 | LUC7L |
| 2354 | 201535_at | 3934.28 | 30.02 | 1.17 | 1.30 | NM_007106 | Hs.145575 | UBL3 |
| 2355 | 210574_s_at | 3934.42 | 30.01 | 1.18 | 1.31 | AF241788 | Hs.263812 | NUDC |
| 2356 | 221861_at | 3934.78 | 30.01 | 1.19 | 1.33 | AL157484 | Hs.12853 | — |
| 2357 | 220338_s_at | 3935.59 | 30.03 | 1.20 | 1.35 | NM_018037 | Hs.220745 | RALGPS2 |
| 2358 | 200733_s_at | 3936.32 | 30.04 | 1.11 | 1.19 | U48296 | Hs.227777 | PTP4A1 |
| 2359 | 208304_at | 3937.24 | 30.06 | 1.16 | 1.28 | NM_001837 | Hs.506190 | CCR3 |
| 2360 | 221671_x_at | 3937.49 | 30.06 | 1.15 | 1.25 | M63438 | Hs.377975 | IGKC |
| 2361 | 212570_at | 3939.86 | 30.13 | 1.07 | 1.12 | AL573201 | Hs.167115 | KIAA0830 |
| 2362 | 202812_at | 3939.96 | 30.12 | 1.11 | 1.18 | NM_000152 | Hs.1437 | GAA |
| 2363 | 219135_s_at | 3940.97 | 30.14 | 1.19 | 1.32 | AL515916 | Hs.58362 | FLJ12681 |
| 2364 | 209620_s_at | 3941.66 | 30.14 | 1.18 | 1.32 | AB005289 | — | — |
| 2365 | 205841_at | 3942.17 | 30.15 | 1.16 | 1.27 | NM_004972 | Hs.434374 | JAK2 |
| 2366 | 218696_at | 3943.75 | 30.19 | 1.20 | 1.34 | NM_004836 | Hs.102506 | EIF2AK3 |
| 2367 | 209340_at | 3945.79 | 30.25 | 1.15 | 1.26 | S73498 | Hs.21293 | UAP1 |
| 2368 | 203474_at | 3946.58 | 30.27 | 1.19 | 1.33 | NM_006633 | Hs.373980 | IQGAP2 |
| 2369 | 212276_at | 3947.51 | 30.29 | 1.12 | 1.21 | D80010 | Hs.81412 | LPIN1 |
| 2370 | 203517_at | 3949.26 | 30.35 | 1.19 | 1.33 | NM_006554 | Hs.31584 | MTX2 |
| 2371 | 212435_at | 3949.78 | 30.35 | 1.18 | 1.32 | AA205593 | Hs.26837 | — |
| 2372 | 213787_s_at | 3949.95 | 30.35 | 1.17 | 1.29 | AV702405 | Hs.196669 | EBP |
| 2373 | 209712_at | 3950.16 | 30.34 | 1.19 | 1.32 | AI769637 | Hs.82635 | SLC35D1 |
| 2374 | 211228_s_at | 3951.13 | 30.36 | 1.20 | 1.35 | AF065736 | Hs.16184 | RAD17 |
| 2375 | 204499_at | 3951.76 | 30.37 | 1.19 | 1.33 | AB028958 | Hs.21542 | AGTPBP1 |
| 2376 | 216064_s_at | 3952.29 | 30.38 | 1.16 | 1.28 | W27131 | — | — |
| 2377 | 201412_at | 3952.62 | 30.37 | 1.16 | 1.28 | NM_014045 | Hs.28368 | LRP10 |
| 2378 | 200643_at | 3952.99 | 30.37 | 1.16 | 1.28 | NM_005336 | Hs.427152 | HDLBP |
| 2379 | 209570_s_at | 3954.11 | 30.40 | 0.95 | 0.93 | BC001745 | Hs.79404 | D4S234E |
| 2380 | 215123_at | 3954.32 | 30.39 | 1.18 | 1.31 | AL049250 | Hs.512019 | KIAA0220 |
| 2381 | 219810_at | 3954.81 | 30.40 | 1.16 | 1.28 | NM_025054 | Hs.287727 | VCIP135 |
| 2382 | 222139_at | 3955.43 | 30.41 | 1.08 | 1.13 | AI765383 | — | KIAA1466 |
| 2383 | 212103_at | 3955.69 | 30.41 | 1.17 | 1.30 | BG403834 | Hs.372741 | KPNA6 |
| 2384 | 202663_at | 3956.17 | 30.41 | 1.17 | 1.30 | AI005043 | Hs.401414 | WASPIP |
| 2385 | 211543_s_at | 3957.69 | 30.45 | 1.19 | 1.33 | AF040752 | Hs.235116 | GRK6 |
| 2386 | 212349_at | 3958.54 | 30.46 | 1.18 | 1.31 | AL045513 | Hs.178292 | POFUT1 |
| 2387 | 208607_s_at | 3959.24 | 30.48 | 1.16 | 1.28 | NM_030754 | Hs.1955 | SAA1 /// SAA2 |
| 2388 | 209316_s_at | 3962.08 | 30.56 | 1.16 | 1.28 | BC001465 | Hs.221040 | HBS1L |
| 2389 | 214122_at | 3962.12 | 30.55 | 1.16 | 1.28 | AA086229 | — | — |
| 2390 | 217027_x_at | 3963.39 | 30.59 | 1.17 | 1.29 | AC004941 | Hs.439683 | KPNB1 |
| 2391 | 217761_at | 3963.47 | 30.58 | 1.16 | 1.28 | NM_018269 | Hs.502773 | MTCBP-1 |
| 2392 | 218999_at | 3964.06 | 30.58 | 1.06 | 1.10 | NM_018295 | Hs.407831 | FLJ11000 |
| 2393 | 202286_s_at | 3966.51 | 30.66 | 0.90 | 0.85 | J04152 | Hs.23582 | TACSTD2 |
| 2394 | 221783_at | 3968.21 | 30.71 | 1.17 | 1.29 | W67467 | Hs.442138 | WIZ |
| 2395 | 209030_s_at | 3968.70 | 30.71 | 1.17 | 1.29 | NM_014333 | Hs.156682 | IGSF4 |
| 2396 | 203221_at | 3969.04 | 30.71 | 1.17 | 1.29 | AI758763 | Hs.406491 | TLE1 |
| 2397 | 210428_s_at | 3969.17 | 30.70 | 1.15 | 1.26 | AF260566 | Hs.416959 | HGS |
| 2398 | 212005_at | 3970.08 | 30.71 | 1.18 | 1.31 | AL582808 | Hs.252967 | DKFZp566C0424 |
| 2399 | 210169_at | 3970.30 | 30.71 | 1.09 | 1.15 | AB007880 | Hs.512856 | KIAA0420 |
| 2400 | 213882_at | 3973.58 | 30.82 | 1.19 | 1.33 | AA012917 | — | — |
| 2401 | 200892_s_at | 3974.14 | 30.83 | 1.13 | 1.22 | BC000451 | Hs.30035 | SFRS10 |
| 2402 | 203344_s_at | 3974.59 | 30.83 | 1.16 | 1.28 | NM_002894 | Hs.437224 | RBBP8 |
| 2403 | 219289_at | 3974.68 | 30.82 | 1.18 | 1.31 | NM_017939 | Hs.313917 | FLJ20718 |
| 2404 | 218425_at | 3975.23 | 30.83 | 1.16 | 1.28 | BC000787 | Hs.336227 | TRIAD3 |
| 2405 | 222073_at | 3976.11 | 30.85 | 1.07 | 1.12 | AI694562 | — | — |
| 2406 | 203006_at | 3976.94 | 30.86 | 1.15 | 1.25 | NM_005539 | Hs.408063 | INPP5A |
| 2407 | 205353_s_at | 3977.00 | 30.85 | 1.17 | 1.30 | NM_002567 | Hs.433863 | PBP |
| 2408 | 212126_at | 3977.69 | 30.86 | 1.19 | 1.33 | BG391282 | Hs.349283 | — |
| 2409 | 213079_at | 3978.76 | 30.89 | 1.18 | 1.31 | AA223871 | Hs.178207 | DT1P1A10 |
| 2410 | 208831_x_at | 3978.92 | 30.88 | 1.18 | 1.30 | D79984 | — | — |
| 2411 | 211798_x_at | 3979.30 | 30.88 | 1.17 | 1.29 | AB001733 | Hs.102950 | IGLJ3 |
| 2412 | 221651_x_at | 3979.49 | 30.88 | 1.16 | 1.27 | BC005332 | Hs.377975 | IGKC |
| 2413 | 205936_s_at | 3980.69 | 30.91 | 1.13 | 1.22 | NM_002115 | Hs.411695 | HK3 |
| 2414 | 213216_at | 3981.58 | 30.93 | 1.18 | 1.32 | AL537463 | Hs.374987 | KIAA0459 |
| 2415 | 204068_at | 3982.52 | 30.95 | 1.14 | 1.24 | NM_006281 | Hs.166684 | STK3 |
| 2416 | 204695_at | 3982.61 | 30.94 | 1.16 | 1.27 | AI343459 | Hs.1634 | CDC25A |
| 2417 | 205526_s_at | 3986.70 | 31.07 | 1.19 | 1.34 | NM_007044 | Hs.440341 | KATNA1 |
| 2418 | 211920_at | 3987.12 | 31.07 | 1.17 | 1.30 | AF349679 | Hs.69771 | BF |
| 2419 | 206255_at | 3987.62 | 31.08 | 1.14 | 1.23 | NM_001715 | Hs.389900 | BLK |
| 2420 | 208679_s_at | 3989.27 | 31.12 | 1.18 | 1.30 | AF279893 | Hs.83583 | ARPC2 |
| 2421 | 212234_at | 3990.12 | 31.13 | 1.19 | 1.33 | AL034550 | Hs.3686 | ASXL1 |
| 2422 | 205041_s_at | 3992.04 | 31.18 | 1.18 | 1.31 | NM_000607 | Hs.278388 | ORM2 |
| 2423 | 214590_s_at | 3993.54 | 31.22 | 1.19 | 1.33 | AL545760 | Hs.129683 | UBE2D1 |
| 2424 | 201944_at | 3993.81 | 31.21 | 1.17 | 1.30 | NM_000521 | Hs.69293 | HEXB |
| 2425 | 213864_s_at | 3993.92 | 31.20 | 1.17 | 1.30 | AI985751 | Hs.419776 | NAP1L1 |
| 2426 | 43544_at | 3994.39 | 31.21 | 1.10 | 1.18 | AA314406 | Hs.365207 | THRAP5 |
| 2427 | 203757_s_at | 3995.88 | 31.25 | 1.23 | 1.40 | BC005008 | Hs.436718 | CEACAM6 |
| 2428 | 201157_s_at | 3996.32 | 31.26 | 1.18 | 1.31 | AF020500 | Hs.346743 | NMT1 |
| 2429 | 203076_s_at | 3996.94 | 31.26 | 1.17 | 1.30 | U65019 | Hs.110741 | SMAD2 |
| 2430 | 212694_s_at | 3997.20 | 31.26 | 1.16 | 1.28 | NM_000532 | Hs.63788 | PCCB |
| 2431 | 222193_at | 4000.08 | 31.35 | 1.18 | 1.32 | AK021672 | Hs.63300 | FLJ21820 |
| 2432 | 218236_s_at | 4000.33 | 31.34 | 1.15 | 1.26 | NM_005813 | Hs.434387 | PRKCN |
| 2433 | 221586_s_at | 4001.63 | 31.37 | 1.18 | 1.31 | U15642 | Hs.447905 | E2F5 |
| 2434 | 200731_s_at | 4002.22 | 31.38 | 1.11 | 1.19 | AW165960 | Hs.227777 | PTP4A1 |
| 2435 | 200903_s_at | 4002.53 | 31.38 | 1.14 | 1.24 | NM_000687 | Hs.388004 | AHCY |
| 2436 | 205147_x_at | 4002.60 | 31.37 | 1.16 | 1.28 | NM_000631 | Hs.196352 | NCF4 |
| 2437 | 213396_s_at | 4002.79 | 31.36 | 1.09 | 1.15 | AA456929 | Hs.372446 | AKAP10 |
| 2438 | 208944_at | 4003.15 | 31.36 | 1.19 | 1.33 | D50683 | — | — |
| 2439 | 210992_x_at | 4003.33 | 31.36 | 1.16 | 1.28 | U90939 | Hs.126384 | FCGR2A /// |
| | | | | | | | | FCGR2B /// |
| | | | | | | | | FCGR2C |
| 2440 | 206816_s_at | 4005.49 | 31.42 | 1.18 | 1.31 | NM_012436 | Hs.256747 | SPAG8 |
| 2441 | 218627_at | 4007.65 | 31.48 | 1.17 | 1.29 | NM_018370 | Hs.416393 | FLJ11259 |
| 2442 | 212163_at | 4007.81 | 31.47 | 1.19 | 1.32 | AB033076 | Hs.9873 | KIDINS220 |
| 2443 | 218268_at | 4009.67 | 31.53 | 1.14 | 1.24 | NM_022771 | Hs.310422 | TBC1D15 |
| 2444 | 209721_s_at | 4010.91 | 31.56 | 1.16 | 1.28 | BC002857 | Hs.15243 | HOM-TES-103 |
| 2445 | 43511_s_at | 4011.52 | 31.57 | 1.19 | 1.33 | AI201594 | Hs.12853 | — |
| 2446 | 202140_s_at | 4013.71 | 31.63 | 1.18 | 1.31 | NM_003992 | Hs.511790 | CLK3 |
| 2447 | 205776_at | 4014.11 | 31.63 | 1.16 | 1.27 | NM_001461 | Hs.396595 | FMO5 |
| 2448 | 205062_x_at | 4014.13 | 31.62 | 1.19 | 1.32 | NM_002892 | Hs.380994 | ARID4A |
| 2449 | 220146_at | 4014.80 | 31.63 | 1.13 | 1.22 | NM_016562 | Hs.179152 | TLR7 |
| 2450 | 200632_s_at | 4016.56 | 31.68 | 1.18 | 1.31 | NM_006096 | Hs.318567 | NDRG1 |
| 2451 | 208804_s_at | 4016.70 | 31.68 | 1.15 | 1.27 | AL031681 | Hs.6891 | SFRS6 |
| 2452 | 202723_s_at | 4017.23 | 31.68 | 1.16 | 1.28 | AW117498 | Hs.170133 | FOXO1A |
| 2453 | 201334_s_at | 4017.35 | 31.68 | 1.17 | 1.30 | AB002380 | Hs.413112 | ARHGEF12 |
| 2454 | 215791_at | 4020.05 | 31.77 | 1.18 | 1.31 | AF003738 | Hs.268526 | — |
| 2455 | 200868_s_at | 4020.20 | 31.76 | 1.16 | 1.27 | NM_018683 | Hs.144949 | ZNF313 |
| 2456 | 209678_s_at | 4020.96 | 31.78 | 1.17 | 1.29 | L18964 | Hs.496511 | PRKCI |
| 2457 | 204502_at | 4021.99 | 31.80 | 1.18 | 1.31 | NM_015474 | Hs.371264 | SAMHD1 |
| 2458 | 219822_at | 4022.23 | 31.80 | 1.16 | 1.27 | NM_004294 | Hs.348472 | MTRF1 |
| 2459 | 217080_s_at | 4022.61 | 31.80 | 1.16 | 1.28 | Y19026 | Hs.93564 | HOMER2 |
| 2460 | 219575_s_at | 4025.33 | 31.88 | 1.18 | 1.31 | NM_022341 | Hs.130849 | COG8 /// PDF |
| 2461 | 203169_at | 4025.82 | 31.88 | 1.18 | 1.31 | NM_014785 | Hs.47313 | KIAA0258 |
| 2462 | 213730_x_at | 4027.79 | 31.94 | 1.18 | 1.31 | BE962186 | Hs.371282 | TCF3 |
| 2463 | 209186_at | 4027.95 | 31.93 | 1.18 | 1.32 | M23114 | Hs.374535 | ATP2A2 |
| 2464 | 219880_at | 4028.26 | 31.93 | 1.19 | 1.33 | NM_022907 | Hs.94037 | FLJ23053 |
| 2465 | 212764_at | 4029.10 | 31.94 | 1.18 | 1.32 | AI806174 | Hs.232068 | TCF8 |
| 2466 | 214146_s_at | 4029.66 | 31.95 | 1.13 | 1.23 | R64130 | Hs.2164 | PPBP |
| 2467 | 213995_at | 4030.97 | 31.99 | 1.14 | 1.25 | AW195882 | Hs.440456 | ATP5S |
| 2468 | 209361_s_at | 4031.42 | 31.99 | 1.17 | 1.30 | BC004153 | Hs.20930 | PCBP4 |
| 2469 | 221042_s_at | 4032.30 | 32.01 | 1.17 | 1.29 | NM_024734 | Hs.301478 | CLMN |
| 2470 | 219316_s_at | 4032.61 | 32.01 | 1.11 | 1.19 | NM_017791 | Hs.267566 | C14orf58 |
| 2471 | 218628_at | 4032.92 | 32.01 | 1.17 | 1.30 | NM_016053 | Hs.18885 | CGI-116 |
| 2472 | 200913_at | 4034.50 | 32.05 | 1.19 | 1.32 | NM_002707 | Hs.17883 | PPM1G |
| 2473 | 209198_s_at | 4035.62 | 32.07 | 1.07 | 1.12 | BC004291 | Hs.380439 | SYT11 |
| 2474 | 209627_s_at | 4035.97 | 32.07 | 1.14 | 1.24 | AY008372 | Hs.197955 | OSBPL3 |
| 2475 | 208454_s_at | 4036.31 | 32.08 | 1.17 | 1.30 | NM_016134 | Hs.197335 | PGCP |
| 2476 | 212780_at | 4036.51 | 32.07 | 1.18 | 1.31 | AA700167 | Hs.326392 | SOS1 |
| 2477 | 218174_s_at | 4036.77 | 32.06 | 1.18 | 1.31 | NM_025125 | Hs.288768 | C10orf57 |
| 2478 | 221068_at | 4037.81 | 32.08 | 1.17 | 1.30 | NM_017616 | Hs.284208 | ANKRD25 |
| 2479 | 220954_s_at | 4038.65 | 32.11 | 1.09 | 1.15 | NM_013440 | Hs.349256 | PILRB |
| 2480 | 210131_x_at | 4039.04 | 32.11 | 1.13 | 1.22 | D49737 | Hs.433982 | SDHC |
| 2481 | 215537_x_at | 4040.44 | 32.14 | 1.17 | 1.29 | AJ012008 | Hs.247362 | DDAH2 |
| 2482 | 215842_s_at | 4040.74 | 32.14 | 1.17 | 1.29 | AK024264 | Hs.29189 | ATP11A |
| 2483 | 221479_s_at | 4042.89 | 32.20 | 1.18 | 1.31 | AF060922 | Hs.132955 | BNIP3L |
| 2484 | 210916_s_at | 4043.23 | 32.20 | 1.17 | 1.30 | AF098641 | Hs.306278 | CD44 |
| 2485 | 218528_s_at | 4043.43 | 32.19 | 1.19 | 1.34 | NM_022781 | Hs.333503 | RNF38 |
| 2486 | 201924_at | 4045.70 | 32.26 | 1.18 | 1.32 | NM_005935 | Hs.114765 | MLLT2 |
| 2487 | 204881_s_at | 4046.20 | 32.26 | 1.15 | 1.26 | NM_003358 | Hs.432605 | UGCG |
| 2488 | 218368_s_at | 4046.26 | 32.25 | 1.16 | 1.28 | NM_016639 | Hs.355899 | TNFRSF12A |
| 2489 | 213511_s_at | 4046.63 | 32.25 | 1.19 | 1.33 | AI167164 | Hs.347187 | MTMR1 |
| 2490 | 208952_s_at | 4046.70 | 32.24 | 1.17 | 1.29 | AA811923 | Hs.192881 | KIAA0217 |
| 2491 | 216510_x_at | 4046.76 | 32.23 | 1.22 | 1.39 | AB035175 | Hs.413826 | IGHG1 |
| 2492 | 204057_at | 4046.87 | 32.22 | 1.17 | 1.29 | AI073984 | — | — |
| 2493 | 211325_x_at | 4048.33 | 32.26 | 1.17 | 1.29 | U72518 | — | LOC171220 |
| 2494 | 220266_s_at | 4048.80 | 32.26 | 1.18 | 1.31 | NM_004235 | Hs.376206 | KLF4 |
| 2495 | 218119_at | 4049.27 | 32.26 | 1.18 | 1.30 | NM_006327 | Hs.11866 | TIMM23 |
| 2496 | 202003_s_at | 4049.41 | 32.25 | 1.17 | 1.29 | NM_006111 | Hs.172506 | ACAA2 |
| 2497 | 208146_s_at | 4050.87 | 32.29 | 1.15 | 1.26 | NM_031311 | Hs.95594 | CPVL |
| 2498 | 200957_s_at | 4051.51 | 32.30 | 1.18 | 1.32 | NM_003146 | Hs.79162 | SSRP1 |
| 2499 | 200030_s_at | 4053.32 | 32.34 | 1.19 | 1.33 | NM_002635 | Hs.290404 | SLC25A3 |
| 2500 | 215200_x_at | 4053.71 | 32.35 | 1.17 | 1.30 | AK022362 | — | — |
| 2501 | 218383_at | 4055.17 | 32.39 | 1.18 | 1.31 | NM_017815 | Hs.442782 | C14orf94 |
| 2502 | 219043_s_at | 4055.29 | 32.38 | 1.19 | 1.33 | NM_024065 | Hs.406178 | LOC285359 /// |
| | | | | | | | | PDCL3 |
| 2503 | 205781_at | 4057.42 | 32.44 | 1.07 | 1.11 | NM_004913 | Hs.164410 | C16orf7 |
| 2504 | 210716_s_at | 4057.65 | 32.44 | 1.18 | 1.32 | M97501 | Hs.410113 | RSN |
| 2505 | 217979_at | 4058.02 | 32.44 | 1.08 | 1.14 | NM_014399 | Hs.364544 | TM4SF13 |
| 2506 | 201963_at | 4059.64 | 32.48 | 1.12 | 1.21 | NM_021122 | Hs.406678 | ACSL1 |
| 2507 | 220953_s_at | 4059.92 | 32.48 | 1.15 | 1.26 | NM_019061 | Hs.93872 | PIP3AP |
| 2508 | 212482_at | 4060.88 | 32.51 | 1.16 | 1.27 | BF671894 | Hs.75277 | FLJ13910 |
| 2509 | 202969_at | 4062.64 | 32.55 | 1.14 | 1.24 | AI216690 | Hs.173135 | DYRK2 |
| 2510 | 201219_at | 4064.03 | 32.58 | 1.14 | 1.24 | AW269836 | Hs.171391 | CTBP2 |
| 2511 | 201255_x_at | 4064.77 | 32.59 | 1.17 | 1.30 | NM_004639 | Hs.440900 | BAT3 |
| 2512 | 202531_at | 4065.62 | 32.61 | 1.11 | 1.19 | NM_002198 | Hs.80645 | IRF1 |
| 2513 | 201591_s_at | 4065.68 | 32.60 | 1.17 | 1.29 | NM_007184 | Hs.435290 | NISCH |
| 2514 | 202948_at | 4066.30 | 32.62 | 1.17 | 1.29 | NM_000877 | Hs.82112 | IL1R1 |
| 2515 | 210962_s_at | 4066.83 | 32.62 | 1.19 | 1.33 | AB019691 | Hs.58103 | AKAP9 |
| 2516 | 211202_s_at | 4067.93 | 32.65 | 1.18 | 1.32 | AF087481 | Hs.143323 | JARID1B |
| 2517 | 208684_at | 4068.43 | 32.65 | 1.18 | 1.31 | U24105 | — | COPA |
| 2518 | 200798_x_at | 4068.69 | 32.65 | 1.14 | 1.23 | NM_021960 | Hs.86386 | MCL1 |
| 2519 | 210069_at | 4070.54 | 32.70 | 1.13 | 1.22 | U62733 | Hs.439777 | CPT1B |
| 2520 | 213319_s_at | 4071.14 | 32.71 | 1.16 | 1.28 | AW170359 | — | — |
| 2521 | 200015_s_at | 4071.50 | 32.71 | 1.17 | 1.29 | NM_004404 | Hs.131736 | NEDD5 |
| 2522 | 208979_at | 4072.40 | 32.73 | 1.19 | 1.32 | AF128458 | Hs.435788 | NCOA6 |
| 2523 | 211974_x_at | 4072.45 | 32.72 | 1.18 | 1.31 | AL513759 | Hs.347340 | RBPSUH |
| 2524 | 219854_at | 4073.10 | 32.73 | 1.13 | 1.22 | NM_021030 | Hs.197219 | ZNF14 |
| 2525 | 217886_at | 4075.84 | 32.81 | 1.19 | 1.34 | BF213575 | Hs.79095 | EPS15 |
| 2526 | 216194_s_at | 4077.47 | 32.86 | 1.18 | 1.32 | AD001527 | Hs.31053 | CKAP1 |
| 2527 | 206631_at | 4078.01 | 32.87 | 1.06 | 1.11 | NM_000956 | Hs.2090 | PTGER2 |
| 2528 | 210556_at | 4078.23 | 32.86 | 1.09 | 1.15 | U85430 | Hs.172674 | NFATC3 |
| 2529 | 202320_at | 4079.18 | 32.88 | 1.17 | 1.30 | NM_001520 | Hs.331 | GTF3C1 |
| 2530 | 200803_s_at | 4079.30 | 32.87 | 1.14 | 1.24 | AF033095 | Hs.35052 | TEGT |
| 2531 | 210184_at | 4079.92 | 32.88 | 1.11 | 1.18 | M81695 | Hs.385521 | ITGAX |
| 2532 | 221900_at | 4080.23 | 32.88 | 1.17 | 1.29 | AI806793 | Hs.353001 | COL8A2 |
| 2533 | 214697_s_at | 4080.70 | 32.88 | 1.14 | 1.24 | AW190873 | Hs.374634 | ROD1 |
| 2534 | 208699_x_at | 4081.37 | 32.89 | 1.17 | 1.30 | BF696840 | Hs.89643 | TKT |
| 2535 | 221840_at | 4081.80 | 32.90 | 1.18 | 1.30 | AA775177 | Hs.437980 | PTPRE |
| 2536 | 212485_at | 4082.25 | 32.90 | 1.16 | 1.28 | AU146596 | Hs.396047 | KIAA0553 |
| 2537 | 211385_x_at | 4083.56 | 32.94 | 1.16 | 1.27 | U28169 | Hs.389536 | SULT1A2 |
| 2538 | 212381_at | 4084.07 | 32.94 | 1.17 | 1.30 | BF444943 | Hs.7243 | USP24 |
| 2539 | 212726_at | 4084.23 | 32.94 | 1.18 | 1.32 | AB014562 | Hs.93868 | PHF2 |
| 2540 | 212406_s_at | 4084.36 | 32.93 | 1.17 | 1.30 | AB028973 | Hs.184628 | C20orf36 |
| 2541 | 212176_at | 4086.12 | 32.99 | 1.15 | 1.26 | AA902326 | Hs.414993 | C6orf111 |
| 2542 | 208913_at | 4088.29 | 33.05 | 1.18 | 1.31 | AA868560 | Hs.133340 | GGA2 |
| 2543 | 213005_s_at | 4089.39 | 33.08 | 1.10 | 1.18 | D79994 | Hs.77546 | ANKRD15 |
| 2544 | 219629_at | 4089.65 | 33.07 | 1.10 | 1.16 | NM_017911 | Hs.265018 | C22orf8 |
| 2545 | 202700_s_at | 4089.74 | 33.06 | 1.17 | 1.29 | NM_014698 | Hs.119387 | KIAA0792 |
| 2546 | 210438_x_at | 4091.67 | 33.12 | 1.18 | 1.31 | M25077 | Hs.288178 | SSA2 |
| 2547 | 222146_s_at | 4092.46 | 33.14 | 1.16 | 1.28 | AK026674 | Hs.359289 | TCF4 |
| 2548 | 217891_at | 4093.25 | 33.16 | 1.17 | 1.30 | NM_022744 | Hs.9003 | FLJ13868 |
| 2549 | 211841_s_at | 4093.67 | 33.17 | 1.14 | 1.24 | U94510 | Hs.299558 | TNFRSF25 |
| 2550 | 219594_at | 4093.92 | 33.16 | 1.15 | 1.25 | NM_016533 | Hs.380976 | NINJ2 |
| 2551 | 211997_x_at | 4094.45 | 33.17 | 1.15 | 1.25 | NM_005324 | Hs.180877 | H3F3B |
| 2552 | 205049_s_at | 4094.72 | 33.17 | 1.13 | 1.23 | NM_001783 | Hs.79630 | CD79A |
| 2553 | 214092_x_at | 4095.01 | 33.16 | 1.17 | 1.29 | AI928127 | Hs.134093 | SFRS14 |
| 2554 | 204028_s_at | 4095.07 | 33.15 | 1.18 | 1.31 | NM_012197 | — | RABGAP1 |
| 2555 | 215859_at | 4095.13 | 33.14 | 1.17 | 1.30 | AK022309 | Hs.465589 | — |
| 2556 | 212794_s_at | 4095.41 | 33.13 | 1.10 | 1.18 | AK001728 | Hs.12144 | KIAA1033 |
| 2557 | 217148_x_at | 4096.52 | 33.16 | 1.12 | 1.21 | AJ249377 | Hs.449592 /// | IGLJ3 /// IGLC2 |
| | | | | | | | Hs.458262 | |
| 2558 | 204811_s_at | 4097.79 | 33.19 | 1.15 | 1.26 | NM_006030 | Hs.389415 | CACNA2D2 |
| 2559 | 220570_at | 4097.95 | 33.19 | 1.14 | 1.23 | NM_020415 | Hs.283091 | RETN |
| 2560 | 210432_s_at | 4098.26 | 33.19 | 1.07 | 1.12 | AF225986 | Hs.300717 | SCN3A |
| 2561 | 204214_s_at | 4101.46 | 33.30 | 1.11 | 1.19 | NM_006834 | Hs.32217 | RAB32 |
| 2562 | 202787_s_at | 4101.84 | 33.30 | 1.07 | 1.12 | U43784 | Hs.234521 | MAPKAPK3 |
| 2563 | 201155_s_at | 4101.88 | 33.29 | 1.16 | 1.28 | NM_014874 | Hs.376681 | MFN2 |
| 2564 | 213170_at | 4102.18 | 33.28 | 1.14 | 1.23 | AA406605 | Hs.43728 | GPX7 |
| 2565 | 221776_s_at | 4102.69 | 33.29 | 1.16 | 1.28 | AI885109 | — | — |
| 2566 | 218099_at | 4103.70 | 33.31 | 1.17 | 1.30 | NM_018469 | Hs.16206 | HT008 |
| 2567 | 205715_at | 4103.99 | 33.31 | 1.17 | 1.29 | NM_004334 | Hs.169998 | BST1 |
| 2568 | 211342_x_at | 4105.58 | 33.34 | 1.18 | 1.31 | BC004354 | Hs.409226 | TNRC11 |
| 2569 | 212371_at | 4109.33 | 33.46 | 1.19 | 1.33 | AL049397 | Hs.211473 | PNAS-4 |
| 2570 | 38241_at | 4109.65 | 33.46 | 1.12 | 1.20 | U90548 | Hs.167741 | BTN3A3 |
| 2571 | 215116_s_at | 4110.88 | 33.49 | 1.16 | 1.27 | AF035321 | Hs.436132 | DNM1 |
| 2572 | 208896_at | 4114.55 | 33.62 | 1.18 | 1.32 | X98743 | Hs.363492 | DDX18 |
| 2573 | 210983_s_at | 4115.99 | 33.67 | 1.15 | 1.26 | AF279900 | Hs.438720 | MCM7 |
| 2574 | 201443_s_at | 4117.16 | 33.70 | 1.18 | 1.31 | AF248966 | Hs.183434 | ATP6AP2 |
| 2575 | 214202_at | 4117.84 | 33.71 | 1.18 | 1.31 | N21364 | Hs.26968 | — |
| 2576 | 200649_at | 4119.64 | 33.76 | 1.16 | 1.28 | BC002356 | Hs.172609 | NUCB1 |
| 2577 | 203042_at | 4120.58 | 33.78 | 1.13 | 1.23 | NM_002294 | Hs.232432 | LAMP2 |
| 2578 | 203555_at | 4120.73 | 33.77 | 1.14 | 1.24 | NM_014369 | Hs.210913 | PTPN18 |
| 2579 | 209266_s_at | 4120.83 | 33.76 | 1.15 | 1.26 | AW134794 | Hs.284205 | SLC39A8 |
| 2580 | 205786_s_at | 4120.91 | 33.75 | 1.17 | 1.29 | NM_000632 | Hs.172631 | ITGAM |
| 2581 | 221654_s_at | 4122.60 | 33.80 | 1.18 | 1.30 | AF077040 | Hs.251636 | USP3 |
| 2582 | 217294_s_at | 4123.04 | 33.80 | 1.16 | 1.27 | U88968 | Hs.433455 | ENO1 |
| 2583 | 218009_s_at | 4123.22 | 33.80 | 1.16 | 1.28 | NM_003981 | Hs.344037 | PRC1 |
| 2584 | 212329_at | 4123.83 | 33.80 | 1.16 | 1.27 | D83782 | Hs.437096 | SCAP |
| 2585 | 217896_s_at | 4125.40 | 33.84 | 1.19 | 1.32 | NM_024946 | Hs.285017 | NIP30 |
| 2586 | 221737_at | 4125.86 | 33.84 | 1.16 | 1.27 | AK024696 | Hs.182874 | GNA12 |
| 2587 | 201952_at | 4126.99 | 33.88 | 1.12 | 1.20 | AA156721 | Hs.10247 | ALCAM |
| 2588 | 204620_s_at | 4128.59 | 33.92 | 1.16 | 1.28 | NM_004385 | Hs.434488 | CSPG2 |
| 2589 | 203408_s_at | 4128.83 | 33.92 | 1.15 | 1.26 | NM_002971 | Hs.416026 | SATB1 |
| 2590 | 209448_at | 4129.45 | 33.93 | 1.15 | 1.25 | BC002439 | Hs.90753 | HTATIP2 |
| 2591 | 201196_s_at | 4130.10 | 33.94 | 1.08 | 1.14 | M21154 | Hs.159118 | AMD1 |
| 2592 | 212688_at | 4133.80 | 34.06 | 1.15 | 1.25 | BC003393 | Hs.239818 | PIK3CB |
| 2593 | 209342_s_at | 4133.87 | 34.04 | 1.18 | 1.31 | AF080158 | Hs.413513 | IKBKB |
| 2594 | 212665_at | 4134.11 | 34.04 | 1.14 | 1.24 | AL556438 | Hs.12813 | TIPARP |
| 2595 | 210314_x_at | 4136.02 | 34.09 | 1.14 | 1.24 | AF114013 | Hs.54673 | TNFSF12- |
| | | | | | | | | TNFSF13 /// |
| | | | | | | | | TNFSF13 |
| 2596 | 220617_s_at | 4138.39 | 34.16 | 1.17 | 1.29 | NM_018181 | Hs.368756 | ZNF532 |
| 2597 | 200983_x_at | 4138.86 | 34.16 | 1.17 | 1.29 | BF983379 | Hs.278573 | CD59 |
| 2598 | 216680_s_at | 4138.98 | 34.15 | 1.15 | 1.26 | BC004264 | Hs.437008 | EPHB4 |
| 2599 | 213505_s_at | 4139.33 | 34.15 | 1.18 | 1.30 | BG252853 | Hs.134093 | SFRS14 |
| 2600 | 204183_s_at | 4139.37 | 34.14 | 1.13 | 1.22 | AI478542 | Hs.13944 | ADRBK2 |
| 2601 | 205035_at | 4139.38 | 34.13 | 1.17 | 1.29 | NM_004715 | Hs.4076 | CTDP1 |
| 2602 | 200597_at | 4139.57 | 34.12 | 1.16 | 1.28 | AI123320 | Hs.389559 | EIF3S10 |
| 2603 | 222371_at | 4140.10 | 34.12 | 1.16 | 1.28 | AI732802 | — | — |
| 2604 | 204162_at | 4140.28 | 34.12 | 1.17 | 1.29 | NM_006101 | Hs.414407 | KNTC2 |
| 2605 | 217403_s_at | 4141.35 | 34.14 | 1.16 | 1.28 | AC074331 | Hs.371335 | ZNF227 |
| 2606 | 213666_at | 4141.78 | 34.14 | 1.14 | 1.24 | AK026589 | Hs.90998 | SEPT6 |
| 2607 | 207769_s_at | 4142.10 | 34.14 | 1.15 | 1.26 | NM_005710 | Hs.30570 | PQBP1 |
| 2608 | 211979_at | 4142.22 | 34.14 | 1.16 | 1.28 | AB046844 | Hs.442329 | GPR107 |
| 2609 | 201537_s_at | 4142.96 | 34.15 | 1.14 | 1.25 | BC002682 | Hs.181046 | DUSP3 |
| 2610 | 201755_at | 4143.32 | 34.15 | 1.17 | 1.29 | NM_006739 | Hs.77171 | MCM5 |
| 2611 | 201971_s_at | 4143.66 | 34.15 | 1.17 | 1.29 | NM_001690 | Hs.409131 | ATP6V1A |
| 2612 | 211944_at | 4144.21 | 34.15 | 1.15 | 1.26 | BE729523 | Hs.446197 | XTP2 |
| 2613 | 220748_s_at | 4144.45 | 34.15 | 1.16 | 1.27 | NM_016202 | Hs.94392 | ZNF580 |
| 2614 | 206073_at | 4144.60 | 34.14 | 1.10 | 1.17 | AF057036 | Hs.138520 | COLQ |
| 2615 | 211615_s_at | 4144.62 | 34.13 | 1.17 | 1.30 | M92439 | Hs.182490 | LRPPRC |
| 2616 | 220023_at | 4145.19 | 34.14 | 1.17 | 1.29 | NM_018690 | Hs.454666 | — |
| 2617 | 210340_s_at | 4145.55 | 34.14 | 1.15 | 1.26 | BC002635 | Hs.520937 | CSF2RA |
| 2618 | 213093_at | 4146.33 | 34.16 | 1.14 | 1.24 | AI471375 | Hs.349611 | PRKCA |
| 2619 | 203466_at | 4146.42 | 34.15 | 1.19 | 1.33 | NM_002437 | Hs.75659 | MPV17 |
| 2620 | 217759_at | 4147.41 | 34.17 | 1.19 | 1.33 | BF431488 | Hs.14512 | TRIM44 |
| 2621 | 201354_s_at | 4147.58 | 34.16 | 1.15 | 1.27 | AA788652 | — | — |
| 2622 | 219731_at | 4147.93 | 34.16 | 1.17 | 1.30 | NM_024343 | — | FLJ34077 |
| 2623 | 214408_s_at | 4148.37 | 34.17 | 1.17 | 1.29 | AL021937 | — | RFPL1S /// |
| | | | | | | | | RFPL3S |
| 2624 | 208908_s_at | 4148.79 | 34.17 | 1.15 | 1.26 | AF327443 | Hs.440961 | CAST |
| 2625 | 219938_s_at | 4148.87 | 34.16 | 1.00 | 1.00 | NM_024430 | Hs.413501 | PSTPIP2 |
| 2626 | 202258_s_at | 4149.06 | 34.15 | 1.18 | 1.31 | U50532 | Hs.131727 | PFAAP5 |
| 2627 | 205504_at | 4149.84 | 34.17 | 1.14 | 1.25 | NM_000061 | Hs.159494 | BTK |
| 2628 | 201330_at | 4150.55 | 34.18 | 1.18 | 1.31 | NM_002887 | Hs.180832 | RARS |
| 2629 | 205512_s_at | 4150.88 | 34.18 | 1.16 | 1.27 | NM_004208 | Hs.18720 | PDCD8 |
| 2630 | 201536_at | 4151.06 | 34.17 | 1.11 | 1.19 | AL048503 | Hs.181046 | DUSP3 |
| 2631 | 201575_at | 4153.39 | 34.23 | 1.17 | 1.30 | NM_012245 | Hs.445498 | SKIIP |
| 2632 | 203181_x_at | 4156.62 | 34.34 | 1.17 | 1.29 | AW149364 | Hs.221688 | SRPK2 |
| 2633 | 206759_at | 4158.04 | 34.38 | 1.16 | 1.28 | NM_002002 | Hs.1416 | FCER2 |
| 2634 | 216768_x_at | 4158.18 | 34.37 | 1.15 | 1.26 | AK025188 | Hs.435832 | FLJ20699 |
| 2635 | 212862_at | 4158.37 | 34.36 | 1.16 | 1.27 | AL568982 | Hs.306912 | CDS2 |
| 2636 | 207091_at | 4161.81 | 34.47 | 1.13 | 1.21 | NM_002562 | Hs.528636 | P2RX7 |
| 2637 | 208527_x_at | 4162.55 | 34.49 | 1.04 | 1.06 | NM_003523 | — | HIST1H2BE |
| 2638 | 209062_x_at | 4163.39 | 34.51 | 1.15 | 1.25 | AF010227 | Hs.382168 | NCOA3 |
| 2639 | 203518_at | 4163.64 | 34.51 | 1.18 | 1.31 | NM_000081 | Hs.130188 | CHS1 |
| 2640 | 216094_at | 4163.77 | 34.50 | 1.07 | 1.12 | AK025323 | Hs.230999 | — |
| 2641 | 212893_at | 4163.99 | 34.50 | 1.15 | 1.26 | AL080063 | Hs.5364 | ZZZ3 |
| 2642 | 207554_x_at | 4166.16 | 34.56 | 1.14 | 1.23 | NM_001060 | — | — |
| 2643 | 204330_s_at | 4167.19 | 34.58 | 1.16 | 1.28 | AA587905 | Hs.411125 | MRPS12 |
| 2644 | 212368_at | 4167.75 | 34.59 | 1.16 | 1.28 | AA972711 | Hs.448341 | ZNF292 |
| 2645 | 212350_at | 4167.96 | 34.59 | 1.15 | 1.26 | AB029031 | Hs.372659 | TBC1D1 |
| 2646 | 209883_at | 4168.50 | 34.59 | 1.11 | 1.19 | AF288389 | Hs.387995 | GLT25D2 |
| 2647 | 210014_x_at | 4169.32 | 34.61 | 1.17 | 1.30 | AF023266 | Hs.436405 | IDH3B |
| 2648 | 209165_at | 4169.79 | 34.61 | 1.15 | 1.26 | AF083208 | Hs.311079 | AATF |
| 2649 | 208930_s_at | 4170.34 | 34.62 | 0.94 | 0.90 | BG032366 | Hs.256583 | ILF3 |
| 2650 | 212211_at | 4172.10 | 34.68 | 1.17 | 1.29 | AI986295 | Hs.131059 | ANKRD17 |
| 2651 | 221828_s_at | 4173.54 | 34.72 | 1.16 | 1.28 | AK024432 | Hs.438972 | C9orf28 |
| 2652 | 218932_at | 4174.74 | 34.75 | 1.15 | 1.27 | NM_017953 | Hs.5111 | FLJ20729 |
| 2653 | 203184_at | 4175.67 | 34.77 | 1.13 | 1.21 | NM_001999 | Hs.79432 | FBN2 |
| 2654 | 218131_s_at | 4177.56 | 34.83 | 1.13 | 1.22 | NM_017660 | Hs.118964 | p66alpha |
| 2655 | 218738_s_at | 4178.04 | 34.83 | 1.17 | 1.29 | NM_016271 | Hs.180403 | RNF138 |
| 2656 | 217976_s_at | 4178.66 | 34.84 | 1.15 | 1.25 | NM_016141 | Hs.351916 | DNCLI1 |
| 2657 | 204430_s_at | 4178.97 | 34.84 | 1.12 | 1.20 | NM_003039 | — | — |
| 2658 | 220784_s_at | 4180.24 | 34.88 | 1.15 | 1.26 | NM_021995 | Hs.162200 | UTS2 |
| 2659 | 218263_s_at | 4180.77 | 34.88 | 1.18 | 1.31 | NM_021211 | Hs.25726 | LOC58486 |
| 2660 | 203250_at | 4182.24 | 34.93 | 1.15 | 1.25 | NM_014892 | Hs.3737 | RBM16 |
| 2661 | 207627_s_at | 4183.29 | 34.95 | 1.17 | 1.30 | NM_005653 | Hs.154970 | TFCP2 |
| 2662 | 37950_at | 4184.70 | 34.99 | 1.15 | 1.26 | X74496 | Hs.433986 | PREP |
| 2663 | 212223_at | 4184.89 | 34.98 | 1.18 | 1.31 | AI926544 | Hs.303154 | IDS |
| 2664 | 200678_x_at | 4185.28 | 34.98 | 1.14 | 1.24 | NM_002087 | — | — |
| 2665 | 208743_s_at | 4185.39 | 34.98 | 1.12 | 1.21 | BC001359 | Hs.279920 | YWHAB |
| 2666 | 220757_s_at | 4186.84 | 35.02 | 1.14 | 1.23 | NM_025241 | Hs.435255 | UBXD1 |
| 2667 | 203063_at | 4187.01 | 35.01 | 1.16 | 1.27 | NM_014634 | Hs.278441 | PPM1F |
| 2668 | 208403_x_at | 4187.12 | 35.00 | 1.05 | 1.08 | NM_002382 | — | — |
| 2669 | 211908_x_at | 4188.63 | 35.03 | 1.16 | 1.27 | M87268 | Hs.413826 | IGHG1 |
| 2670 | 201456_s_at | 4190.64 | 35.10 | 1.13 | 1.23 | AU160695 | Hs.418533 | BUB3 |
| 2671 | 221809_at | 4193.39 | 35.19 | 1.18 | 1.31 | AB040897 | Hs.6343 | RANBP10 |
| 2672 | 219296_at | 4194.00 | 35.20 | 1.15 | 1.25 | NM_019028 | Hs.163091 | ZDHHC13 |
| 2673 | 37028_at | 4195.79 | 35.25 | 1.03 | 1.05 | U83981 | Hs.76556 | PPP1R15A |
| 2674 | 212291_at | 4196.38 | 35.26 | 1.12 | 1.21 | AI393355 | Hs.12259 | HIPK1 |
| 2675 | 202367_at | 4196.74 | 35.26 | 1.15 | 1.25 | NM_001913 | Hs.438974 | CUTL1 |
| 2676 | 215049_x_at | 4197.35 | 35.27 | 1.16 | 1.28 | Z22969 | Hs.74076 | CD163 |
| 2677 | 200785_s_at | 4198.45 | 35.29 | 1.15 | 1.26 | NM_002332 | Hs.162757 | LRP1 |
| 2678 | 215193_x_at | 4199.21 | 35.31 | 1.16 | 1.28 | AJ297586 | Hs.308026 | HLA-DRB3 |
| 2679 | 205323_s_at | 4200.62 | 35.34 | 1.14 | 1.23 | NM_005955 | Hs.211581 | MTF1 |
| 2680 | 209499_x_at | 4201.14 | 35.35 | 1.14 | 1.24 | BF448647 | Hs.54673 | TNFSF12- |
| | | | | | | | | TNFSF13 /// |
| | | | | | | | | TNFSF13 |
| 2681 | 209545_s_at | 4201.57 | 35.36 | 1.15 | 1.26 | AF064824 | Hs.103755 | RIPK2 |
| 2682 | 216002_at | 4201.63 | 35.35 | 1.17 | 1.30 | AU147200 | Hs.509645 | — |
| 2683 | 211762_s_at | 4201.65 | 35.33 | 1.17 | 1.30 | BC005978 | Hs.159557 | KPNA2 |
| 2684 | 219032_x_at | 4202.90 | 35.37 | 1.17 | 1.29 | NM_014322 | Hs.170129 | OPN3 |
| 2685 | 218888_s_at | 4203.02 | 35.36 | 1.16 | 1.27 | NM_018092 | Hs.6823 | NETO2 |
| 2686 | 203789_s_at | 4203.16 | 35.35 | 1.18 | 1.30 | NM_006379 | Hs.171921 | SEMA3C |
| 2687 | 219622_at | 4203.29 | 35.34 | 1.09 | 1.15 | NM_017817 | Hs.179791 | RAB20 |
| 2688 | 217513_at | 4203.89 | 35.35 | 1.14 | 1.23 | BG334495 | Hs.406258 | LOC284021 |
| 2689 | 212397_at | 4204.05 | 35.35 | 1.17 | 1.29 | AL137751 | Hs.263671 | RDX |
| 2690 | 218971_s_at | 4206.88 | 35.44 | 1.17 | 1.30 | NM_014149 | Hs.512143 | HSPC049 |
| 2691 | 206015_s_at | 4207.66 | 35.45 | 1.18 | 1.30 | NM_014947 | Hs.26023 | FOXJ3 |
| 2692 | 201234_at | 4207.83 | 35.44 | 1.17 | 1.29 | NM_004517 | Hs.6196 | ILK |
| 2693 | 201320_at | 4208.12 | 35.44 | 1.14 | 1.24 | BF663402 | Hs.236030 | SMARCC2 |
| 2694 | 219402_s_at | 4208.90 | 35.46 | 1.15 | 1.26 | NM_024295 | Hs.241576 | DER1 |
| 2695 | 91952_at | 4210.80 | 35.51 | 1.15 | 1.25 | AI363375 | Hs.443636 | LOC90379 |
| 2696 | 211578_s_at | 4210.96 | 35.51 | 1.19 | 1.33 | M60725 | Hs.86858 | RPS6KB1 |
| 2697 | 205333_s_at | 4211.03 | 35.50 | 1.17 | 1.29 | NM_005133 | Hs.528725 | RCE1 |
| 2698 | 217258_x_at | 4212.35 | 35.54 | 1.16 | 1.28 | AF043583 | Hs.449599 | — |
| 2699 | 212160_at | 4212.84 | 35.54 | 1.17 | 1.30 | AI984005 | Hs.85951 | XPOT |
| 2700 | 208029_s_at | 4214.44 | 35.58 | 1.11 | 1.19 | NM_018407 | Hs.296398 | LAPTM4B |
| 2701 | 201940_at | 4215.42 | 35.61 | 1.17 | 1.30 | AA897514 | Hs.5057 | CPD |
| 2702 | 37652_at | 4216.11 | 35.62 | 1.16 | 1.27 | AB002328 | Hs.435798 | CABIN1 |
| 2703 | 209298_s_at | 4216.89 | 35.64 | 1.17 | 1.30 | AF114488 | Hs.66392 | ITSN1 |
| 2704 | 207993_s_at | 4217.11 | 35.63 | 1.16 | 1.28 | NM_007236 | Hs.406234 | CHP |
| 2705 | 206918_s_at | 4220.77 | 35.76 | 1.11 | 1.18 | NM_003915 | Hs.166887 | CPNE1 |
| 2706 | 200700_s_at | 4220.79 | 35.75 | 1.10 | 1.16 | NM_006854 | Hs.446645 | KDELR2 |
| 2707 | 204096_s_at | 4221.44 | 35.76 | 1.16 | 1.27 | AL136771 | Hs.5881 | ELL |
| 2708 | 219104_at | 4221.49 | 35.75 | 1.16 | 1.27 | NM_016422 | Hs.44685 | RNF141 |
| 2709 | 213407_at | 4222.40 | 35.77 | 1.15 | 1.26 | AB023148 | Hs.531564 | KIAA0931 |
| 2710 | 206906_at | 4222.98 | 35.78 | 1.14 | 1.24 | NM_003259 | Hs.151250 | ICAM5 |
| 2711 | 204275_at | 4223.29 | 35.78 | 1.15 | 1.26 | AI796687 | Hs.416262 | SOLH |
| 2712 | 221691_x_at | 4226.80 | 35.89 | 1.18 | 1.31 | AB042278 | Hs.411098 | NPM1 |
| 2713 | 201039_s_at | 4227.59 | 35.90 | 1.11 | 1.18 | BF572938 | Hs.440960 | RAD23A |
| 2714 | 219336_s_at | 4227.79 | 35.89 | 1.17 | 1.30 | NM_015947 | Hs.112694 | ASCC1 |
| 2715 | 218917_s_at | 4228.05 | 35.89 | 1.19 | 1.33 | NM_018450 | Hs.170333 | ARID1A |
| 2716 | 202979_s_at | 4228.36 | 35.89 | 1.12 | 1.20 | NM_021212 | Hs.321022 | ZF |
| 2717 | 208833_s_at | 4228.91 | 35.90 | 1.17 | 1.30 | AF119662 | Hs.191294 | ATXN10 |
| 2718 | 203502_at | 4229.35 | 35.90 | 1.15 | 1.26 | NM_001724 | Hs.198365 | BPGM |
| 2719 | 209902_at | 4230.15 | 35.91 | 1.18 | 1.31 | U49844 | Hs.77613 | ATR |
| 2720 | 210694_s_at | 4230.96 | 35.93 | 1.07 | 1.13 | AF041209 | — | — |
| 2721 | 221572_s_at | 4232.37 | 35.97 | 1.16 | 1.27 | AF288410 | Hs.436194 | SLC26A6 |
| 2722 | 217478_s_at | 4232.45 | 35.96 | 1.17 | 1.29 | X76775 | Hs.351279 | HLA-DMA |
| 2723 | 214786_at | 4236.10 | 36.09 | 1.16 | 1.28 | AA361361 | Hs.170610 | MAP3K1 |
| 2724 | 36920_at | 4236.83 | 36.11 | 1.18 | 1.31 | U46024 | Hs.386205 | MTM1 |
| 2725 | 205570_at | 4236.94 | 36.10 | 1.15 | 1.26 | NM_005028 | Hs.108966 | PIP5K2A |
| 2726 | 205260_s_at | 4239.07 | 36.17 | 1.16 | 1.28 | NM_001107 | Hs.18573 | ACYP1 |
| 2727 | 217995_at | 4239.96 | 36.19 | 1.10 | 1.17 | NM_021199 | Hs.435468 | SQRDL |
| 2728 | 204082_at | 4240.04 | 36.18 | 1.16 | 1.27 | NM_006195 | Hs.294101 | PBX3 |
| 2729 | 221158_at | 4240.16 | 36.17 | 1.14 | 1.24 | NM_013329 | Hs.297559 | C21orf66 |
| 2730 | 209275_s_at | 4243.96 | 36.30 | 1.18 | 1.31 | AF015593 | Hs.194660 | CLN3 |
| 2731 | 207111_at | 4245.04 | 36.32 | 1.10 | 1.17 | NM_001974 | Hs.2375 | EMR1 |
| 2732 | 206272_at | 4245.74 | 36.33 | 1.10 | 1.17 | NM_006542 | Hs.511754 | SPHAR |
| 2733 | 212231_at | 4246.31 | 36.34 | 1.18 | 1.31 | AB020682 | Hs.184227 | FBXO21 |
| 2734 | 202455_at | 4246.54 | 36.34 | 1.15 | 1.27 | NM_005474 | Hs.9028 | HDAC5 |
| 2735 | 221738_at | 4250.65 | 36.48 | 1.16 | 1.28 | BG236163 | Hs.348929 | KIAA1219 |
| 2736 | 211034_s_at | 4250.88 | 36.47 | 1.18 | 1.31 | BC006270 | Hs.7314 | FLJ30092 |
| 2737 | 214741_at | 4251.35 | 36.47 | 1.18 | 1.31 | AW968301 | Hs.97845 | ZNF131 |
| 2738 | 203942_s_at | 4251.92 | 36.49 | 1.14 | 1.24 | NM_017490 | Hs.157199 | MARK2 |
| 2739 | 214777_at | 4253.10 | 36.52 | 1.16 | 1.27 | BG482805 | Hs.534005 | — |
| 2740 | 203884_s_at | 4254.34 | 36.55 | 1.18 | 1.32 | NM_014904 | Hs.173656 | RAB11FIP2 |
| 2741 | 222282_at | 4256.42 | 36.61 | 1.13 | 1.22 | AV761453 | — | — |
| 2742 | 222138_s_at | 4257.18 | 36.63 | 1.17 | 1.30 | AF158978 | Hs.12142 | WDR13 |
| 2743 | 203569_s_at | 4257.50 | 36.63 | 1.15 | 1.27 | NM_003611 | Hs.6483 | OFD1 |
| 2744 | 221229_s_at | 4260.30 | 36.73 | 1.16 | 1.28 | NM_017910 | Hs.32356 | FLJ20628 |
| 2745 | 218739_at | 4260.32 | 36.72 | 1.15 | 1.26 | NM_016006 | Hs.19385 | ABHD5 |
| 2746 | 216841_s_at | 4260.44 | 36.71 | 1.05 | 1.09 | X15132 | Hs.384944 | SOD2 |
| 2747 | 203288_at | 4260.95 | 36.72 | 1.18 | 1.32 | NM_014686 | Hs.436976 | KIAA0355 |
| 2748 | 214977_at | 4261.01 | 36.71 | 1.16 | 1.28 | AK023852 | Hs.492966 | — |
| 2749 | 206338_at | 4263.12 | 36.78 | 1.15 | 1.27 | NM_001420 | Hs.1701 | ELAVL3 |
| 2750 | 205451_at | 4263.23 | 36.77 | 1.16 | 1.27 | NM_005938 | Hs.512585 | MLLT7 |
| 2751 | 221812_at | 4263.29 | 36.76 | 1.16 | 1.28 | AK025059 | Hs.62767 | FBXO42 |
| 2752 | 212847_at | 4265.44 | 36.83 | 1.14 | 1.24 | AL036840 | Hs.22370 | NEXN |
| 2753 | 202692_s_at | 4266.43 | 36.86 | 1.17 | 1.29 | NM_014233 | Hs.89781 | UBTF |
| 2754 | 222131_x_at | 4267.46 | 36.88 | 1.16 | 1.28 | BC004327 | Hs.444178 | RHOT2 |
| 2755 | 212708_at | 4267.68 | 36.88 | 1.13 | 1.22 | AV721987 | — | LOC339287 |
| 2756 | 207530_s_at | 4268.66 | 36.90 | 1.16 | 1.28 | NM_004936 | Hs.72901 | CDKN2B |
| 2757 | 212237_at | 4269.12 | 36.90 | 1.17 | 1.29 | N64780 | Hs.3686 | ASXL1 |
| 2758 | 201853_s_at | 4269.47 | 36.90 | 1.15 | 1.25 | NM_021873 | Hs.153752 | CDC25B |
| 2759 | 201377_at | 4269.78 | 36.90 | 1.16 | 1.27 | NM_014847 | Hs.8127 | UBAP2L |
| 2760 | 215342_s_at | 4270.05 | 36.90 | 1.02 | 1.04 | AB019490 | Hs.242271 | RABGAP1L |
| 2761 | 218031_s_at | 4270.97 | 36.92 | 1.17 | 1.30 | NM_018589 | — | CHES1 |
| 2762 | 220761_s_at | 4271.03 | 36.91 | 1.17 | 1.30 | NM_016281 | Hs.12040 | JIK |
| 2763 | 202130_at | 4271.42 | 36.91 | 1.15 | 1.26 | AA725102 | Hs.209061 | RIOK3 |
| 2764 | 204491_at | 4273.41 | 36.97 | 1.18 | 1.31 | R40917 | Hs.28482 | PDE4D |
| 2765 | 212591_at | 4273.86 | 36.97 | 1.17 | 1.30 | AA887480 | — | — |
| 2766 | 210891_s_at | 4274.56 | 36.98 | 1.15 | 1.26 | AF035737 | Hs.408507 | GTF2I /// GTF2IP1 |
| 2767 | 212257_s_at | 4275.46 | 37.00 | 1.13 | 1.21 | AW131754 | Hs.396404 | SMARCA2 |
| 2768 | 204744_s_at | 4280.22 | 37.15 | 1.18 | 1.30 | NM_013417 | Hs.172801 | IARS |
| 2769 | 221021_s_at | 4280.39 | 37.15 | 1.17 | 1.29 | NM_030877 | Hs.178576 | CTNNBL1 |
| 2770 | 200667_at | 4280.73 | 37.14 | 1.16 | 1.28 | BF448062 | Hs.411826 | UBE2D3 |
| 2771 | 117_at | 4280.83 | 37.13 | 1.14 | 1.24 | X51757 | Hs.3268 | HSPA6 |
| 2772 | 218188_s_at | 4281.60 | 37.15 | 1.12 | 1.21 | NM_012458 | Hs.75056 | TIMM13 |
| 2773 | 201997_s_at | 4281.90 | 37.15 | 1.17 | 1.29 | NM_015001 | Hs.184245 | SHARP |
| 2774 | 211930_at | 4281.91 | 37.13 | 1.14 | 1.24 | AW080932 | Hs.446430 | HNRPA3 |
| 2775 | 210719_s_at | 4282.31 | 37.13 | 1.13 | 1.23 | BC002552 | Hs.406534 | HMG20B |
| 2776 | 213811_x_at | 4283.38 | 37.16 | 1.16 | 1.28 | AW062341 | Hs.371282 | TCF3 |
| 2777 | 201241_at | 4283.69 | 37.16 | 1.16 | 1.28 | NM_004939 | Hs.440599 | DDX1 |
| 2778 | 212238_at | 4284.50 | 37.18 | 1.16 | 1.28 | AL117518 | Hs.3686 | ASXL1 |
| 2779 | 205940_at | 4285.73 | 37.22 | 1.14 | 1.24 | NM_002470 | Hs.440895 | MYH3 |
| 2780 | 206656_s_at | 4286.62 | 37.24 | 1.16 | 1.27 | BC000353 | Hs.22391 | C20orf3 |
| 2781 | 201426_s_at | 4286.85 | 37.23 | 1.14 | 1.24 | AI922599 | Hs.435800 | VIM |
| 2782 | 207522_s_at | 4286.89 | 37.22 | 1.14 | 1.24 | NM_005173 | Hs.5541 | ATP2A3 |
| 2783 | 220112_at | 4287.41 | 37.23 | 1.12 | 1.21 | NM_024669 | Hs.84560 | FLJ11795 |
| 2784 | 205992_s_at | 4287.94 | 37.24 | 1.12 | 1.20 | NM_000585 | Hs.528402 | IL15 |
| 2785 | 214184_at | 4289.07 | 37.27 | 1.16 | 1.27 | AW195837 | Hs.104555 | NPFF |
| 2786 | 209289_at | 4289.78 | 37.28 | 1.10 | 1.16 | AI700518 | Hs.302690 | NFIB |
| 2787 | 211976_at | 4290.40 | 37.29 | 1.17 | 1.29 | AK026168 | Hs.188882 | — |
| 2788 | 212172_at | 4290.56 | 37.28 | 1.10 | 1.17 | AW277253 | Hs.294008 | AK2 |
| 2789 | 203262_s_at | 4290.65 | 37.27 | 1.16 | 1.27 | NM_004699 | Hs.54277 | DXS9928E |
| 2790 | 202396_at | 4291.76 | 37.30 | 1.15 | 1.26 | NM_006706 | Hs.300052 | TCERG1 |
| 2791 | 221811_at | 4292.10 | 37.30 | 1.16 | 1.28 | BF033007 | Hs.91668 | PERLD1 |
| 2792 | 216076_at | 4293.79 | 37.35 | 1.15 | 1.25 | AW445040 | Hs.300863 | L3MBTL |
| 2793 | 218902_at | 4295.38 | 37.39 | 1.10 | 1.17 | NM_017617 | Hs.311559 | NOTCH1 |
| 2794 | 219456_s_at | 4297.99 | 37.46 | 1.17 | 1.30 | AW027923 | Hs.413374 | RIN3 |
| 2795 | 218114_at | 4299.34 | 37.50 | 1.15 | 1.25 | NM_013365 | Hs.405689 | GGA1 |
| 2796 | 212419_at | 4301.38 | 37.57 | 1.14 | 1.24 | AA131324 | Hs.28264 | C10orf56 |
| 2797 | 207549_x_at | 4301.63 | 37.57 | 1.17 | 1.29 | NM_002389 | Hs.83532 | MCP |
| 2798 | 203537_at | 4301.97 | 37.56 | 1.18 | 1.30 | NM_002767 | Hs.13339 | PRPSAP2 |
| 2799 | 212720_at | 4302.61 | 37.57 | 1.1 | 1.17 | AI670847 | Hs.201085 | PAPOLA |
| 2800 | 207168_s_at | 4302.94 | 37.57 | 1.15 | 1.26 | NM_004893 | Hs.75258 | H2AFY |
| 2801 | 214077_x_at | 4303.29 | 37.57 | 1.15 | 1.26 | H15129 | Hs.356135 | MEIS4 |
| 2802 | 201422_at | 4304.57 | 37.61 | 1.13 | 1.22 | NM_006332 | Hs.14623 | IFI30 |
| 2803 | 214960_at | 4305.92 | 37.64 | 1.17 | 1.30 | AF229253 | Hs.444340 | API5 |
| 2804 | 210592_s_at | 4308.76 | 37.74 | 1.12 | 1.20 | M55580 | Hs.28491 | SAT |
| 2805 | 215749_s_at | 4309.47 | 37.76 | 1.16 | 1.27 | AK001574 | Hs.250181 | GORASP1 |
| 2806 | 218650_at | 4311.20 | 37.81 | 1.08 | 1.13 | NM_022775 | Hs.350243 | DGCR8 |
| 2807 | 204037_at | 4311.71 | 37.82 | 1.13 | 1.22 | BF055366 | Hs.75794 | EDG2 |
| 2808 | 209869_at | 4311.90 | 37.81 | 1.12 | 1.21 | AF284095 | Hs.249159 | ADRA2A |
| 2809 | 220059_at | 4312.59 | 37.83 | 1.11 | 1.19 | NM_012108 | Hs.121128 | BRDG1 |
| 2810 | 204900_x_at | 4313.92 | 37.87 | 1.15 | 1.25 | NM_003864 | Hs.512813 | SAP30 |
| 2811 | 208614_s_at | 4314.29 | 37.87 | 1.15 | 1.26 | M62994 | Hs.81008 | FLNB |
| 2812 | 200920_s_at | 4314.44 | 37.86 | 1.17 | 1.30 | AL535380 | — | — |
| 2813 | 201737_s_at | 4314.53 | 37.85 | 1.14 | 1.24 | NM_005885 | Hs.380875 | TEB4 |
| 2814 | 218311_at | 4314.84 | 37.84 | 1.18 | 1.31 | NM_003618 | — | — |
| 2815 | 218016_s_at | 4315.55 | 37.86 | 1.17 | 1.30 | NM_018119 | Hs.355714 | POLR3E |
| 2816 | 212019_at | 4315.75 | 37.85 | 1.15 | 1.26 | AK025446 | Hs.528354 | DKFZP564M182 |
| 2817 | 205281_s_at | 4316.17 | 37.85 | 1.16 | 1.27 | NM_002641 | Hs.51 | PIGA |
| 2818 | 209871_s_at | 4320.03 | 37.98 | 1.08 | 1.14 | AB014719 | Hs.26468 | APBA2 |
| 2819 | 219349_s_at | 4320.32 | 37.98 | 1.15 | 1.25 | NM_018303 | Hs.436664 | SEC5L1 |
| 2820 | 201956_s_at | 4321.06 | 37.99 | 1.17 | 1.30 | NM_014236 | Hs.12482 | GNPAT |
| 2821 | 212010_s_at | 4321.69 | 38.00 | 1.17 | 1.29 | AK025647 | Hs.283690 | H41 |
| 2822 | 214790_at | 4322.48 | 38.02 | 1.16 | 1.27 | AK001406 | Hs.435628 | SENP6 |
| 2823 | 200974_at | 4323.36 | 38.04 | 1.14 | 1.23 | NM_001613 | Hs.208641 | ACTA2 |
| 2824 | 219497_s_at | 4325.21 | 38.09 | 1.15 | 1.26 | NM_022893 | Hs.314623 | BCL11A |
| 2825 | 211084_x_at | 4325.81 | 38.10 | 1.17 | 1.30 | Z25429 | Hs.434387 | PRKCN |
| 2826 | 204445_s_at | 4326.55 | 38.11 | 1.17 | 1.29 | AI361850 | Hs.89499 | ALOX5 |
| 2827 | 216850_at | 4328.76 | 38.19 | 1.14 | 1.24 | AF319523 | Hs.48375 | SNRPN |
| 2828 | 209002_s_at | 4329.37 | 38.19 | 1.16 | 1.28 | BC003177 | Hs.156667 | KIAA1536 |
| 2829 | 203611_at | 4329.52 | 38.18 | 1.17 | 1.30 | NM_005652 | Hs.63335 | TERF2 |
| 2830 | 217122_s_at | 4331.80 | 38.25 | 1.17 | 1.30 | AL031282 | Hs.214646 | SLC35E2 |
| 2831 | 203324_s_at | 4332.47 | 38.26 | 1.15 | 1.26 | NM_001233 | Hs.139851 | CAV2 |
| 2832 | 212080_at | 4332.52 | 38.25 | 1.18 | 1.31 | AV714029 | Hs.258855 | MLL |
| 2833 | 214909_s_at | 4333.60 | 38.28 | 1.15 | 1.26 | AK026191 | Hs.247362 | DDAH2 |
| 2834 | 206474_at | 4333.74 | 38.27 | 1.14 | 1.24 | NM_002595 | Hs.258536 | PCTK2 |
| 2835 | 213159_at | 4334.80 | 38.30 | 1.17 | 1.29 | AB018348 | Hs.55947 | PCNX |
| 2836 | 217208_s_at | 4335.55 | 38.31 | 1.17 | 1.29 | AL121981 | Hs.389893 | DLG1 |
| 2837 | 211671_s_at | 4335.96 | 38.32 | 1.18 | 1.31 | U01351 | Hs.126608 | NR3C1 |
| 2838 | 217915_s_at | 4336.41 | 38.32 | 1.15 | 1.26 | NM_016304 | Hs.274772 | C15orf15 |
| 2839 | 54037_at | 4336.61 | 38.32 | 1.17 | 1.30 | AL041451 | Hs.441481 | HPS4 |
| 2840 | 212197_x_at | 4336.94 | 38.32 | 1.17 | 1.29 | AB020671 | Hs.430725 | M-RIP |
| 2841 | 210154_at | 4339.25 | 38.38 | 1.16 | 1.27 | M55905 | Hs.75342 | ME2 |
| 2842 | 210773_s_at | 4341.26 | 38.44 | 1.13 | 1.21 | U81501 | Hs.99855 | FPRL1 |
| 2843 | 201047_x_at | 4341.61 | 38.44 | 1.16 | 1.28 | BC003617 | Hs.5636 | RAB6A |
| 2844 | 214126_at | 4343.03 | 38.48 | 1.16 | 1.28 | N39314 | Hs.46791 | MCART1 |
| 2845 | 217179_x_at | 4344.92 | 38.53 | 1.15 | 1.25 | X79782 | Hs.440830 | — |
| 2846 | 220968_s_at | 4345.62 | 38.55 | 1.14 | 1.24 | NM_031285 | Hs.108557 | PP1057 |
| 2847 | 212702_s_at | 4346.34 | 38.56 | 1.08 | 1.14 | N45111 | Hs.436939 | BICD2 |
| 2848 | 221524_s_at | 4346.54 | 38.55 | 1.15 | 1.25 | AF272036 | Hs.238679 | RRAGD |
| 2849 | 216241_s_at | 4346.62 | 38.54 | 1.15 | 1.26 | X57198 | Hs.78869 | TCEA1 |
| 2850 | 202326_at | 4347.25 | 38.55 | 1.16 | 1.28 | NM_006709 | Hs.75196 | BAT8 |
| 2851 | 214867_at | 4347.30 | 38.54 | 1.13 | 1.23 | AL080182 | Hs.225129 | NDST2 |
| 2852 | 218970_s_at | 4347.68 | 38.54 | 1.17 | 1.30 | NM_015960 | Hs.16606 | CUTC |
| 2853 | 202816_s_at | 4347.74 | 38.53 | 1.17 | 1.29 | AW292882 | Hs.404263 | SS18 |
| 2854 | 206284_x_at | 4347.93 | 38.52 | 1.13 | 1.22 | NM_001834 | Hs.380749 | CLTB |
| 2855 | 204391_x_at | 4349.78 | 38.58 | 1.15 | 1.26 | NM_015905 | Hs.183858 | TIF1 |
| 2856 | 212377_s_at | 4350.23 | 38.58 | 1.16 | 1.27 | AU158495 | Hs.8121 | NOTCH2 |
| 2857 | 205763_s_at | 4350.75 | 38.59 | 1.16 | 1.27 | NM_006773 | Hs.363492 | DDX18 |
| 2858 | 213160_at | 4351.73 | 38.61 | 1.15 | 1.26 | D86964 | Hs.17211 | DOCK2 |
| 2859 | 201726_at | 4351.83 | 38.61 | 1.16 | 1.27 | BC003376 | Hs.184492 | ELAVL1 |
| 2860 | 221293_s_at | 4352.75 | 38.63 | 1.18 | 1.30 | NM_022047 | Hs.15476 | DEF6 |
| 2861 | 200599_s_at | 4353.04 | 38.63 | 1.16 | 1.28 | NM_003299 | Hs.192374 | TRA1 |
| 2862 | 221922_at | 4353.72 | 38.64 | 1.16 | 1.27 | AW195581 | Hs.278338 | GPSM2 |
| 2863 | 200673_at | 4353.74 | 38.63 | 1.16 | 1.27 | NM_014713 | Hs.111894 | LAPTM4A |
| 2864 | 207085_x_at | 4353.83 | 38.62 | 1.13 | 1.23 | NM_006140 | Hs.520937 | CSF2RA |
| 2865 | 214938_x_at | 4354.81 | 38.64 | 1.16 | 1.28 | AF283771 | Hs.434102 | HMGB1 |
| 2866 | 203734_at | 4355.34 | 38.65 | 1.17 | 1.29 | NM_018416 | Hs.120844 | FOXJ2 |
| 2867 | 207740_s_at | 4355.53 | 38.64 | 1.16 | 1.27 | NM_012346 | Hs.528342 | NUP62 |
| 2868 | 209767_s_at | 4355.88 | 38.64 | 1.07 | 1.11 | AA702163 | Hs.283743 | GP1BB /// PNUTL1 |
| 2869 | 208859_s_at | 4357.27 | 38.67 | 0.99 | 0.98 | AI650257 | Hs.440734 | ATRX |
| 2870 | 220642_x_at | 4358.47 | 38.71 | 1.18 | 1.31 | NM_016334 | Hs.412059 | GPR89 |
| 2871 | 212000_at | 4359.49 | 38.73 | 1.15 | 1.26 | AB002363 | Hs.134093 | SFRS14 |
| 2872 | 220777_at | 4360.76 | 38.76 | 1.08 | 1.13 | NM_022113 | Hs.146286 | KIF13A |
| 2873 | 209150_s_at | 4362.19 | 38.80 | 1.16 | 1.28 | U94831 | Hs.91586 | TM9SF1 |
| 2874 | 217074_at | 4363.13 | 38.83 | 1.14 | 1.25 | AK025938 | Hs.433337 | SMOX |
| 2875 | 201484_at | 4364.81 | 38.89 | 1.13 | 1.23 | NM_003168 | Hs.79058 | SUPT4H1 |
| 2876 | 218037_at | 4364.92 | 38.87 | 1.16 | 1.27 | NM_024293 | Hs.22412 | C2orf17 |
| 2877 | 211052_s_at | 4365.45 | 38.88 | 1.15 | 1.26 | BC006364 | Hs.435541 | TBCD |
| 2878 | 221992_at | 4365.66 | 38.87 | 1.10 | 1.17 | AI925734 | Hs.165328 | LOC283970 |
| 2879 | 212335_at | 4366.44 | 38.88 | 1.12 | 1.20 | AW167793 | Hs.334534 | GNS |
| 2880 | 209884_s_at | 4366.70 | 38.88 | 1.16 | 1.28 | AF047033 | Hs.250072 | SLC4A7 |
| 2881 | 210427_x_at | 4367.49 | 38.90 | 1.14 | 1.23 | BC001388 | Hs.462864 | ANXA2 |
| 2882 | 201925_s_at | 4369.67 | 38.97 | 1.11 | 1.19 | NM_000574 | Hs.408864 | DAF |
| 2883 | 216306_x_at | 4371.19 | 39.01 | 1.17 | 1.30 | X62006 | Hs.172550 | PTBP1 |
| 2884 | 204054_at | 4371.20 | 39.00 | 1.17 | 1.29 | NM_000314 | Hs.253309 | PTEN |
| 2885 | 208870_x_at | 4372.18 | 39.02 | 1.12 | 1.20 | BC000931 | — | — |
| 2886 | 208639_x_at | 4372.79 | 39.03 | 1.11 | 1.18 | BC001312 | Hs.212102 | TXNDC7 |
| 2887 | 204059_s_at | 4374.94 | 39.10 | 1.13 | 1.22 | NM_002395 | Hs.14732 | ME1 |
| 2888 | 209912_s_at | 4375.05 | 39.09 | 1.14 | 1.23 | AI373854 | Hs.505758 | FOXK1 |
| 2889 | 201161_s_at | 4377.52 | 39.17 | 1.15 | 1.26 | NM_003651 | Hs.221889 | CSDA |
| 2890 | 212811_x_at | 4377.59 | 39.16 | 1.15 | 1.25 | AI889380 | Hs.323878 | SLC1A4 |
| 2891 | 209290_s_at | 4379.60 | 39.21 | 1.07 | 1.11 | BC001283 | Hs.302690 | NFIB |
| 2892 | 210638_s_at | 4379.62 | 39.20 | 1.15 | 1.27 | AF176704 | Hs.388387 | FBXO9 |
| 2893 | 202266_at | 4379.70 | 39.19 | 1.15 | 1.25 | NM_016614 | Hs.210628 | TTRAP |
| 2894 | 209046_s_at | 4382.13 | 39.26 | 1.16 | 1.27 | AB030710 | Hs.6518 | GABARAPL2 |
| 2895 | 211594_s_at | 4382.73 | 39.27 | 1.16 | 1.28 | AB049636 | Hs.288936 | MRPL9 |
| 2896 | 219304_s_at | 4382.80 | 39.26 | 1.04 | 1.06 | NM_025208 | Hs.112885 | PDGFD |
| 2897 | 211810_s_at | 4383.20 | 39.26 | 1.14 | 1.24 | D25284 | Hs.408273 | GALC |
| 2898 | 200948_at | 4383.20 | 39.24 | 1.15 | 1.27 | NM_005439 | Hs.79026 | MLF2 |
| 2899 | 201631_s_at | 4385.16 | 39.31 | 1.02 | 1.04 | NM_003897 | Hs.76095 | IER3 |
| 2900 | 201943_s_at | 4388.22 | 39.41 | 1.16 | 1.27 | NM_001304 | Hs.5057 | CPD |
| 2901 | 200900_s_at | 4388.56 | 39.41 | 1.05 | 1.09 | AI583537 | Hs.134084 | M6PR |
| 2902 | 200598_s_at | 4388.57 | 39.39 | 1.06 | 1.10 | AI582238 | Hs.192374 | TRA1 |
| 2903 | 207475_at | 4390.05 | 39.44 | 1.15 | 1.25 | NM_000134 | Hs.282265 | FABP2 |
| 2904 | 213175_s_at | 4390.05 | 39.43 | 1.09 | 1.15 | AL049650 | Hs.83753 | SNRPB |
| 2905 | 203447_at | 4390.33 | 39.42 | 1.17 | 1.29 | AU157008 | Hs.193725 | PSMD5 |
| 2906 | 218854_at | 4390.46 | 39.41 | 1.12 | 1.21 | NM_013352 | Hs.388014 | SART2 |
| 2907 | 209285_s_at | 4390.98 | 39.42 | 1.16 | 1.28 | N38985 | Hs.23440 | RAP140 |
| 2908 | 212463_at | 4391.08 | 39.41 | 1.14 | 1.24 | BE379006 | Hs.278573 | CD59 |
| 2909 | 211168_s_at | 4391.30 | 39.41 | 1.15 | 1.26 | D86988 | Hs.388125 | RENT1 |
| 2910 | 214882_s_at | 4391.85 | 39.42 | 1.17 | 1.30 | BG254869 | Hs.73965 | SFRS2 |
| 2911 | 215778_x_at | 4395.32 | 39.54 | 1.15 | 1.26 | AJ006206 | Hs.183096 | HAB1 |
| 2912 | 203839_s_at | 4396.56 | 39.57 | 1.13 | 1.22 | NM_005781 | Hs.528296 | ACK1 |
| 2913 | 214795_at | 4396.58 | 39.55 | 1.14 | 1.23 | AL137703 | Hs.37372 | PRKCBP1 |
| 2914 | 211251_x_at | 4396.63 | 39.54 | 1.17 | 1.29 | U78774 | Hs.285133 | NFYC |
| 2915 | 203265_s_at | 4396.89 | 39.54 | 1.17 | 1.29 | AA810268 | Hs.134106 | MAP2K4 |
| 2916 | 204568_at | 4397.91 | 39.57 | 1.16 | 1.28 | NM_014924 | Hs.414809 | KIAA0831 |
| 2917 | 202616_s_at | 4398.60 | 39.59 | 1.17 | 1.30 | AI631140 | Hs.3239 | MECP2 |
| 2918 | 210039_s_at | 4401.01 | 39.65 | 1.15 | 1.25 | L01087 | Hs.408049 | PRKCQ |
| 2919 | 221163_s_at | 4401.63 | 39.66 | 1.15 | 1.25 | NM_015977 | Hs.285681 | WBSCR14 |
| 2920 | 206011_at | 4402.74 | 39.69 | 1.11 | 1.19 | AI719655 | Hs.2490 | CASP1 |
| 2921 | 212416_at | 4402.79 | 39.68 | 1.17 | 1.30 | AV745949 | Hs.31218 | SCAMP1 |
| 2922 | 219869_s_at | 4403.48 | 39.70 | 1.13 | 1.21 | NM_022154 | Hs.284205 | SLC39A8 |
| 2923 | 203501_at | 4404.30 | 39.71 | 1.10 | 1.17 | NM_006102 | Hs.197335 | PGCP |
| 2924 | 214728_x_at | 4404.35 | 39.70 | 1.15 | 1.26 | AK026573 | Hs.78202 | SMARCA4 |
| 2925 | 205895_s_at | 4404.90 | 39.71 | 1.15 | 1.25 | NM_004741 | Hs.75337 | NOLC1 |
| 2926 | 218066_at | 4405.33 | 39.71 | 1.10 | 1.17 | NM_006598 | Hs.172613 | SLC12A7 |
| 2927 | 218857_s_at | 4406.90 | 39.76 | 1.16 | 1.27 | NM_025080 | Hs.433197 | ASRGL1 |
| 2928 | 200615_s_at | 4407.13 | 39.75 | 1.13 | 1.23 | AL567295 | Hs.370123 | AP2B1 |
| 2929 | 210718_s_at | 4407.43 | 39.76 | 0.96 | 0.94 | AF119889 | Hs.58323 | LOC51326 |
| 2930 | 212956_at | 4407.99 | 39.77 | 1.08 | 1.13 | AI348094 | Hs.411317 | KIAA0882 |
| 2931 | 206019_at | 4408.28 | 39.76 | 1.15 | 1.25 | NM_014852 | Hs.7482 | RBM19 |
| 2932 | 201734_at | 4412.56 | 39.91 | 1.15 | 1.25 | AI760629 | Hs.372528 | CLCN3 |
| 2933 | 213476_x_at | 4413.75 | 39.95 | 1.15 | 1.26 | AL565749 | Hs.511743 | TUBB4 |
| 2934 | 200883_at | 4413.91 | 39.94 | 1.11 | 1.18 | NM_003366 | Hs.173554 | UQCRC2 |
| 2935 | 211383_s_at | 4414.22 | 39.94 | 1.18 | 1.30 | AL136827 | Hs.435671 | WDR37 |
| 2936 | 210361_s_at | 4415.00 | 39.95 | 1.16 | 1.27 | AF256223 | Hs.82143 | ELF2 |
| 2937 | 217873_at | 4416.79 | 40.02 | 1.16 | 1.28 | NM_016289 | Hs.6406 | CAB39 |
| 2938 | 202725_at | 4417.06 | 40.02 | 1.13 | 1.23 | NM_000937 | Hs.171880 | POLR2A |
| 2939 | 222034_at | 4417.60 | 40.03 | 1.16 | 1.27 | AA443762 | Hs.5662 | GNB2L1 |
| 2940 | 201624_at | 4418.26 | 40.04 | 1.16 | 1.27 | NM_001349 | Hs.32393 | DARS |
| 2941 | 201724_s_at | 4420.03 | 40.09 | 1.17 | 1.30 | NM_020474 | Hs.277324 | GALNT1 |
| 2942 | 208683_at | 4420.72 | 40.11 | 1.15 | 1.26 | M23254 | Hs.350899 | CAPN2 |
| 2943 | 215780_s_at | 4421.11 | 40.11 | 0.96 | 0.94 | Z95126 | Hs.436687 | SET |
| 2944 | 218219_s_at | 4421.84 | 40.13 | 1.15 | 1.26 | NM_018697 | Hs.134342 | LANCL2 |
| 2945 | 208705_s_at | 4425.30 | 40.25 | 1.15 | 1.26 | BG481972 | Hs.433702 | EIF5 |
| 2946 | 203961_at | 4425.36 | 40.24 | 0.89 | 0.83 | AL157398 | Hs.5025 | NEBL |
| 2947 | 212609_s_at | 4425.71 | 40.24 | 1.16 | 1.27 | U79271 | Hs.511758 | SDCCAG8 |
| 2948 | 214450_at | 4425.81 | 40.23 | 1.03 | 1.04 | NM_001335 | Hs.416848 | CTSW |
| 2949 | 201534_s_at | 4426.74 | 40.25 | 1.17 | 1.29 | AF044221 | Hs.145575 | UBL3 |
| 2950 | 215666_at | 4430.49 | 40.37 | 1.15 | 1.25 | U70544 | — | HLA-DRB4 |
| 2951 | 214108_at | 4430.75 | 40.37 | 1.04 | 1.07 | AI346181 | Hs.42712 | MAX |
| 2952 | 206316_s_at | 4430.91 | 40.36 | 1.14 | 1.25 | NM_014708 | Hs.300559 | KNTC1 |
| 2953 | 208935_s_at | 4430.93 | 40.35 | 1.18 | 1.31 | L78132 | Hs.4082 | LGALS8 |
| 2954 | 208309_s_at | 4431.95 | 40.37 | 1.16 | 1.27 | NM_006785 | Hs.180566 | MALT1 |
| 2955 | 209579_s_at | 4433.57 | 40.42 | 1.15 | 1.26 | AL556619 | Hs.35947 | MBD4 |
| 2956 | 211692_s_at | 4434.10 | 40.42 | 1.14 | 1.23 | AF332558 | Hs.87246 | BBC3 |
| 2957 | 203759_at | 4436.17 | 40.49 | 1.14 | 1.24 | NM_006278 | Hs.75268 | SIAT4C |
| 2958 | 218863_s_at | 4438.01 | 40.54 | 1.15 | 1.25 | NM_022648 | Hs.439442 | TNS |
| 2959 | 201137_s_at | 4438.18 | 40.54 | 1.13 | 1.21 | NM_002121 | Hs.368409 | HLA-DPB1 |
| 2960 | 208914_at | 4440.52 | 40.62 | 1.16 | 1.27 | BE646414 | Hs.133340 | GGA2 |
| 2961 | 213081_at | 4440.78 | 40.62 | 1.15 | 1.26 | AL523144 | Hs.206770 | ZNF297 |
| 2962 | 210153_s_at | 4440.96 | 40.61 | 1.16 | 1.28 | M55905 | Hs.75342 | ME2 |
| 2963 | 207304_at | 4441.42 | 40.62 | 1.17 | 1.29 | NM_003425 | Hs.381285 | ZNF45 |
| 2964 | 208624_s_at | 4441.68 | 40.61 | 1.05 | 1.09 | BE966878 | Hs.433750 | EIF4G1 |
| 2965 | 202059_s_at | 4441.71 | 40.60 | 1.15 | 1.26 | NM_002264 | Hs.161008 | KPNA1 |
| 2966 | 33494_at | 4442.81 | 40.63 | 1.14 | 1.24 | S69232 | Hs.511919 | ETFDH |
| 2967 | 204026_s_at | 4443.00 | 40.62 | 1.16 | 1.27 | NM_007057 | Hs.42650 | ZWINT |
| 2968 | 202225_at | 4443.79 | 40.64 | 1.17 | 1.29 | AW612311 | Hs.343220 | — |
| 2969 | 203775_at | 4444.07 | 40.64 | 1.13 | 1.21 | NM_014251 | Hs.9599 | SLC25A13 |
| 2970 | 201864_at | 4445.13 | 40.66 | 1.15 | 1.25 | NM_001493 | Hs.74576 | GDI1 |
| 2971 | 220117_at | 4447.18 | 40.73 | 1.12 | 1.21 | NM_024697 | Hs.99256 | FLJ22419 |
| 2972 | 201546_at | 4448.70 | 40.77 | 1.17 | 1.29 | NM_004238 | Hs.388373 | TRIP12 |
| 2973 | 204888_s_at | 4449.10 | 40.77 | 1.16 | 1.28 | AA772093 | Hs.146233 | NEURL |
| 2974 | 204142_at | 4449.37 | 40.77 | 1.14 | 1.25 | NM_017512 | Hs.475848 | HSRTSBETA |
| 2975 | 207098_s_at | 4450.63 | 40.80 | 1.15 | 1.25 | NM_017927 | Hs.433673 | MFN1 |
| 2976 | 212580_at | 4451.20 | 40.81 | 1.16 | 1.28 | BG111635 | Hs.440961 | CAST |
| 2977 | 201837_s_at | 4452.01 | 40.82 | 1.17 | 1.29 | AF197954 | Hs.6232 | STAF65(gamma) |
| 2978 | 216628_at | 4452.25 | 40.81 | 1.15 | 1.26 | AL117447 | — | — |
| 2979 | 208613_s_at | 4454.79 | 40.89 | 1.15 | 1.26 | AV712733 | Hs.81008 | FLNB |
| 2980 | 214959_s_at | 4455.42 | 40.90 | 1.16 | 1.28 | AF229253 | Hs.444340 | API5 |
| 2981 | 209515_s_at | 4456.58 | 40.93 | 1.15 | 1.26 | U38654 | Hs.298530 | RAB27A |
| 2982 | 202128_at | 4457.33 | 40.95 | 1.15 | 1.26 | NM_014821 | Hs.434432 | KIAA0317 |
| 2983 | 211156_at | 4458.02 | 40.97 | 1.15 | 1.25 | AF115544 | Hs.421349 | CDKN2A |
| 2984 | 204393_s_at | 4459.54 | 41.01 | 1.08 | 1.13 | NM_001099 | Hs.388677 | ACPP |
| 2985 | 220998_s_at | 4459.60 | 41.00 | 1.14 | 1.24 | NM_030930 | Hs.135187 | UNC93B1 |
| 2986 | 218386_x_at | 4463.10 | 41.13 | 1.17 | 1.30 | NM_006447 | Hs.99819 | USP16 |
| 2987 | 217831_s_at | 4465.06 | 41.20 | 1.16 | 1.27 | NM_016143 | Hs.12865 | NSFL1C |
| 2988 | 205718_at | 4465.89 | 41.23 | 1.14 | 1.25 | NM_000889 | Hs.1741 | ITGB7 |
| 2989 | 202452_at | 4466.63 | 41.25 | 1.15 | 1.26 | AI991574 | Hs.29285 | C9orf60 |
| 2990 | 200993_at | 4467.10 | 41.26 | 1.17 | 1.30 | AA939270 | Hs.434408 | IPO7 |
| 2991 | 211284_s_at | 4468.70 | 41.31 | 1.15 | 1.25 | BC000324 | Hs.180577 | GRN |
| 2992 | 201113_at | 4468.97 | 41.31 | 1.15 | 1.25 | NM_003321 | Hs.12084 | TUFM |
| 2993 | 219095_at | 4472.26 | 41.43 | 1.13 | 1.22 | NM_005090 | Hs.198161 | PLA2G4B |
| 2994 | 202629_at | 4472.70 | 41.44 | 1.17 | 1.29 | AV681579 | Hs.84084 | APPBP2 |
| 2995 | 204435_at | 4473.76 | 41.47 | 1.12 | 1.21 | NM_014778 | Hs.406243 | NUPL1 |
| 2996 | 201623_s_at | 4474.36 | 41.48 | 1.15 | 1.26 | BC000629 | Hs.32393 | DARS |
| 2997 | 207616_s_at | 4474.38 | 41.47 | 1.16 | 1.27 | NM_004180 | Hs.146847 | TANK |
| 2998 | 212056_at | 4475.12 | 41.48 | 1.16 | 1.28 | D80004 | Hs.222171 | KIAA0182 |
| 2999 | 209799_at | 4475.84 | 41.50 | 1.16 | 1.27 | AF100763 | Hs.43322 | PRKAA1 |
| 3000 | 200078_s_at | 4476.49 | 41.51 | 1.13 | 1.22 | BC005876 | Hs.7476 | ATP6V0B |
| 3001 | 212632_at | 4477.15 | 41.52 | 1.15 | 1.26 | N32035 | Hs.152335 | — |
| 3002 | 219131_at | 4477.77 | 41.53 | 1.16 | 1.27 | NM_013319 | Hs.443567 | TERE1 |
| 3003 | 211102_s_at | 4478.83 | 41.56 | 1.15 | 1.25 | U82277 | — | LILRA2 |
| 3004 | 204587_at | 4480.84 | 41.62 | 1.16 | 1.27 | NM_003951 | Hs.194686 | SLC25A14 |
| 3005 | 202515_at | 4481.84 | 41.65 | 1.17 | 1.29 | BG251175 | Hs.389893 | DLG1 |
| 3006 | 202732_at | 4482.03 | 41.64 | 1.11 | 1.18 | NM_007066 | Hs.3407 | PKIG |
| 3007 | 208184_s_at | 4482.04 | 41.63 | 1.15 | 1.27 | NM_003274 | Hs.438927 | TMEM1 |
| 3008 | 218807_at | 4483.30 | 41.66 | 1.15 | 1.26 | NM_006113 | Hs.267659 | VAV3 |
| 3009 | 218885_s_at | 4483.45 | 41.65 | 1.15 | 1.26 | NM_024642 | Hs.47099 | GALNT12 |
| 3010 | 210658_s_at | 4484.77 | 41.69 | 1.16 | 1.28 | BC000284 | Hs.133340 | GGA2 |
| 3011 | 203651_at | 4485.59 | 41.71 | 1.14 | 1.23 | NM_014733 | Hs.438240 | ZFYVE16 |
| 3012 | 203175_at | 4486.25 | 41.72 | 1.14 | 1.24 | NM_001665 | Hs.75082 | RHOG |
| 3013 | 203771_s_at | 4486.69 | 41.72 | 1.14 | 1.24 | AA740186 | Hs.435726 | BLVRA |
| 3014 | 216980_s_at | 4486.86 | 41.72 | 1.14 | 1.25 | X60502 | — | — |
| 3015 | 209953_s_at | 4489.24 | 41.80 | 1.16 | 1.27 | U63131 | Hs.160958 | CDC37 |
| 3016 | 211373_s_at | 4489.58 | 41.81 | 1.14 | 1.24 | U34349 | Hs.25363 | PSEN2 |
| 3017 | 200816_s_at | 4490.12 | 41.81 | 1.17 | 1.30 | NM_000430 | Hs.77318 | PAFAH1B1 |
| 3018 | 206894_at | 4490.15 | 41.80 | 1.15 | 1.26 | NM_000482 | Hs.1247 | APOA4 |
| 3019 | 213482_at | 4491.13 | 41.82 | 1.16 | 1.28 | BF593175 | Hs.7022 | DOCK3 |
| 3020 | 221207_s_at | 4491.50 | 41.82 | 1.15 | 1.25 | NM_015678 | Hs.3821 | NBEA |
| 3021 | 213684_s_at | 4491.68 | 41.82 | 1.11 | 1.18 | BF671400 | Hs.154103 | LIM |
| 3022 | 202016_at | 4491.99 | 41.82 | 1.10 | 1.16 | NM_002402 | Hs.440459 | MEST |
| 3023 | 212061_at | 4492.86 | 41.84 | 1.16 | 1.27 | AB002330 | Hs.370907 | SR140 |
| 3024 | 204730_at | 4493.69 | 41.86 | 1.16 | 1.28 | NM_014747 | Hs.78748 | RIMS3 |
| 3025 | 203236_s_at | 4494.03 | 41.86 | 1.12 | 1.21 | NM_009587 | Hs.81337 | LGALS9 |
| 3026 | 205223_at | 4494.22 | 41.85 | 1.16 | 1.27 | NM_014662 | Hs.435022 | DEPDC5 |
| 3027 | 202594_at | 4494.36 | 41.84 | 1.08 | 1.13 | NM_015344 | Hs.146585 | LEPROTL1 |
| 3028 | 208657_s_at | 4495.12 | 41.86 | 1.12 | 1.20 | AF142408 | Hs.288094 | MSF |
| 3029 | 201847_at | 4495.17 | 41.85 | 1.11 | 1.19 | NM_000235 | Hs.85226 | LIPA |
| 3030 | 209536_s_at | 4496.54 | 41.88 | 1.16 | 1.27 | AF320070 | Hs.55058 | EHD4 |
| 3031 | 203392_s_at | 4498.26 | 41.93 | 1.13 | 1.22 | NM_001328 | Hs.196083 | CTBP1 |
| 3032 | 206374_at | 4498.55 | 41.93 | 1.16 | 1.27 | NM_004420 | Hs.511698 | DUSP8 |
| 3033 | 210338_s_at | 4498.68 | 41.93 | 1.02 | 1.03 | AB034951 | Hs.180414 | HSPA8 |
| 3034 | 207320_x_at | 4501.21 | 42.03 | 1.15 | 1.26 | NM_004602 | Hs.6113 | STAU |
| 3035 | 216705_s_at | 4501.62 | 42.03 | 1.13 | 1.22 | X02189 | Hs.407135 | ADA |
| 3036 | 214173_x_at | 4501.79 | 42.02 | 1.15 | 1.26 | AW514900 | Hs.7943 | C19orf2 |
| 3037 | 208269_s_at | 4503.04 | 42.06 | 1.15 | 1.27 | NM_021777 | Hs.528304 | ADAM28 |
| 3038 | 217966_s_at | 4503.91 | 42.08 | 1.03 | 1.04 | NM_022083 | Hs.48778 | C1orf24 |
| 3039 | 218167_at | 4504.07 | 42.07 | 1.16 | 1.28 | NM_016627 | Hs.268122 | LOC51321 |
| 3040 | 221194_s_at | 4504.08 | 42.06 | 1.15 | 1.26 | NM_016125 | Hs.30154 | LOC51136 |
| 3041 | 214531_s_at | 4504.35 | 42.06 | 1.14 | 1.25 | AF065484 | Hs.498154 | SNX1 |
| 3042 | 210186_s_at | 4504.80 | 42.06 | 1.14 | 1.24 | BC005147 | Hs.374638 | FKBP1A |
| 3043 | 209158_s_at | 4504.99 | 42.05 | 1.17 | 1.28 | BC004361 | Hs.8517 | PSCD2 |
| 3044 | 202470_s_at | 4505.87 | 42.07 | 1.15 | 1.25 | NM_007007 | Hs.64542 | CPSF6 |
| 3045 | 200932_s_at | 4507.68 | 42.13 | 1.15 | 1.26 | NM_006400 | Hs.289123 | DCTN2 |
| 3046 | 202361_at | 4509.27 | 42.18 | 1.15 | 1.25 | NM_004922 | Hs.534042 | SEC24C |
| 3047 | 208278_s_at | 4511.13 | 42.23 | 1.15 | 1.25 | NM_016420 | — | — |
| 3048 | 213045_at | 4511.88 | 42.25 | 1.16 | 1.28 | AB011133 | Hs.173864 | MAST3 |
| 3049 | 216071_x_at | 4512.20 | 42.25 | 1.16 | 1.27 | AF132033 | Hs.409226 | TNRC11 |
| 3050 | 209765_at | 4512.31 | 42.24 | 1.15 | 1.25 | Y13786 | Hs.289368 | ADAM19 |
| 3051 | 203914_x_at | 4514.17 | 42.30 | 1.11 | 1.18 | NM_000860 | Hs.77348 | HPGD |
| 3052 | 203729_at | 4515.10 | 42.32 | 1.15 | 1.27 | NM_001425 | Hs.9999 | EMP3 |
| 3053 | 212178_s_at | 4515.32 | 42.32 | 1.13 | 1.23 | AK022555 | Hs.380964 | POM121 /// |
| | | | | | | | | LOC340318 |
| 3054 | 213696_s_at | 4516.73 | 42.36 | 1.16 | 1.28 | AA421957 | Hs.301756 | MED8 |
| 3055 | 202810_at | 4516.79 | 42.35 | 1.13 | 1.21 | NM_004147 | Hs.115242 | DRG1 |
| 3056 | 208835_s_at | 4517.31 | 42.36 | 1.07 | 1.12 | AW089673 | Hs.130293 | CROP |
| 3057 | 202636_at | 4517.64 | 42.36 | 1.17 | 1.29 | NM_005667 | Hs.147159 | RNF103 |
| 3058 | 213326_at | 4519.30 | 42.41 | 1.12 | 1.20 | AU150319 | Hs.20021 | VAMP1 |
| 3059 | 202985_s_at | 4520.20 | 42.43 | 1.15 | 1.25 | NM_004873 | Hs.5443 | BAG5 |
| 3060 | 202662_s_at | 4521.39 | 42.47 | 1.16 | 1.28 | NM_002223 | Hs.512235 | ITPR2 |
| 3061 | 218401_s_at | 4522.01 | 42.48 | 1.14 | 1.24 | NM_012482 | Hs.59757 | ZNF281 |
| 3062 | 202691_at | 4522.01 | 42.46 | 1.13 | 1.23 | NM_006938 | Hs.86948 | SNRPD1 |
| 3063 | 205382_s_at | 4522.99 | 42.49 | 1.04 | 1.07 | NM_001928 | Hs.155597 | DF |
| 3064 | 213271_s_at | 4525.45 | 42.58 | 1.16 | 1.28 | AB029040 | Hs.348982 | KIAA1117 |
| 3065 | 215399_s_at | 4525.79 | 42.58 | 1.13 | 1.21 | AI683900 | Hs.76228 | OS-9 |
| 3066 | 201339_s_at | 4526.78 | 42.60 | 1.13 | 1.23 | NM_002979 | Hs.75760 | SCP2 |
| 3067 | 211714_x_at | 4527.22 | 42.61 | 1.13 | 1.23 | BC005838 | Hs.356729 | OK/SW-cl.56 |
| 3068 | 218516_s_at | 4527.62 | 42.61 | 1.15 | 1.27 | NM_017813 | Hs.378857 | FLJ20421 |
| 3069 | 214671_s_at | 4528.04 | 42.62 | 1.16 | 1.28 | L19704 | Hs.434004 | ABR |
| 3070 | 216984_x_at | 4528.59 | 42.63 | 1.11 | 1.18 | D84143 | — | — |
| 3071 | 212212_s_at | 4529.37 | 42.64 | 1.13 | 1.23 | BF055496 | Hs.112184 | DKFZP586J0619 |
| 3072 | 205253_at | 4529.49 | 42.63 | 1.05 | 1.08 | NM_002585 | Hs.408222 | PBX1 |
| 3073 | 220091_at | 4531.96 | 42.72 | 1.09 | 1.15 | NM_017585 | Hs.244378 | SLC2A6 |
| 3074 | 221681_s_at | 4532.31 | 42.72 | 1.15 | 1.25 | AF094508 | Hs.447902 | DSPP |
| 3075 | 210140_at | 4532.58 | 42.72 | 0.99 | 0.98 | AF031824 | Hs.143212 | CST7 |
| 3076 | 211061_s_at | 4532.66 | 42.71 | 1.14 | 1.25 | BC006390 | Hs.93338 | MGAT2 |
| 3077 | 208669_s_at | 4533.22 | 42.72 | 1.16 | 1.27 | AF109873 | Hs.381137 | CRI1 |
| 3078 | 200845_s_at | 4533.52 | 42.72 | 1.07 | 1.12 | NM_004905 | Hs.120 | PRDX6 |
| 3079 | 209754_s_at | 4534.44 | 42.74 | 1.12 | 1.21 | AF113682 | Hs.11355 | TMPO |
| 3080 | 218271_s_at | 4535.32 | 42.76 | 1.14 | 1.24 | NM_018622 | Hs.13094 | PSARL |
| 3081 | 203099_s_at | 4535.89 | 42.76 | 1.11 | 1.18 | AF081258 | Hs.16081 | CDYL |
| 3082 | 218067_s_at | 4536.52 | 42.78 | 1.13 | 1.22 | NM_018011 | Hs.406114 | FLJ10154 |
| 3083 | 208290_s_at | 4536.64 | 42.77 | 1.12 | 1.21 | NM_001969 | Hs.433702 | EIF5 |
| 3084 | 221499_s_at | 4537.52 | 42.78 | 1.13 | 1.23 | AK026970 | Hs.349074 | STX16 |
| 3085 | 200009_at | 4537.79 | 42.78 | 1.16 | 1.27 | NM_001494 | Hs.56845 | GDI2 |
| 3086 | 203514_at | 4540.00 | 42.85 | 1.12 | 1.20 | BF971923 | Hs.29282 | MAP3K3 |
| 3087 | 201729_s_at | 4541.46 | 42.89 | 1.15 | 1.26 | NM_014680 | Hs.151761 | KIAA0100 |
| 3088 | 201590_x_at | 4543.90 | 42.97 | 1.15 | 1.25 | NM_004039 | Hs.462864 | ANXA2 |
| 3089 | 214523_at | 4545.06 | 43.00 | 1.15 | 1.25 | NM_001805 | Hs.426867 | CEBPE |
| 3090 | 201803_at | 4545.67 | 43.01 | 1.17 | 1.28 | NM_000938 | Hs.149353 | POLR2B |
| 3091 | 200757_s_at | 4546.27 | 43.02 | 1.15 | 1.26 | NM_001219 | Hs.7753 | CALU |
| 3092 | 202250_s_at | 4546.87 | 43.03 | 1.14 | 1.25 | NM_015726 | Hs.120904 | WDR42A |
| 3093 | 202370_s_at | 4548.01 | 43.07 | 1.13 | 1.23 | NM_001755 | Hs.179881 | CBFB |
| 3094 | 208055_s_at | 4549.23 | 43.10 | 1.15 | 1.27 | NM_015601 | Hs.96867 | HERC4 |
| 3095 | 210309_at | 4549.97 | 43.12 | 1.15 | 1.26 | AB042823 | Hs.33818 | RECQL5 |
| 3096 | 203769_s_at | 4550.64 | 43.14 | 1.15 | 1.25 | NM_000351 | Hs.79876 | STS |
| 3097 | 200760_s_at | 4551.23 | 43.15 | 1.00 | 0.99 | N92494 | Hs.92384 | ARL6IP5 |
| 3098 | 208195_at | 4551.33 | 43.14 | 1.11 | 1.19 | NM_003319 | Hs.434384 | TTN |
| 3099 | 221847_at | 4552.04 | 43.16 | 1.13 | 1.22 | BF665706 | — | — |
| 3100 | 212958_x_at | 4553.90 | 43.21 | 1.15 | 1.25 | AI022882 | Hs.352733 | PAM |
| 3101 | 216142_at | 4554.03 | 43.21 | 1.16 | 1.27 | AL137403 | Hs.429565 | — |
| 3102 | 216472_at | 4554.63 | 43.21 | 1.14 | 1.24 | AF003737 | Hs.517244 | — |
| 3103 | 210947_s_at | 4555.07 | 43.21 | 1.11 | 1.20 | J04810 | Hs.42674 | MSH3 |
| 3104 | 202962_at | 4555.07 | 43.20 | 1.14 | 1.24 | NM_015254 | Hs.15711 | KIF13B |
| 3105 | 207163_s_at | 4555.13 | 43.19 | 1.13 | 1.23 | NM_005163 | Hs.368861 | AKT1 |
| 3106 | 218429_s_at | 4556.90 | 43.24 | 1.15 | 1.27 | NM_018381 | Hs.175120 | FLJ11286 |
| 3107 | 212208_at | 4557.55 | 43.26 | 1.10 | 1.16 | AK023837 | Hs.435249 | THRAP2 |
| 3108 | 203100_s_at | 4559.20 | 43.31 | 1.16 | 1.28 | NM_004824 | Hs.16081 | CDYL |
| 3109 | 212415_at | 4560.78 | 43.36 | 1.08 | 1.13 | AW150913 | Hs.90998 | SEPT6 |
| 3110 | 216163_at | 4562.40 | 43.41 | 1.14 | 1.24 | AK025086 | Hs.449291 | HBLD2 |
| 3111 | 206267_s_at | 4562.93 | 43.42 | 1.11 | 1.19 | NM_002378 | Hs.437808 | MATK |
| 3112 | 210281_s_at | 4562.95 | 43.40 | 1.16 | 1.27 | AL136621 | Hs.315241 | ZNF198 |
| 3113 | 206589_at | 4566.08 | 43.51 | 1.12 | 1.20 | NM_005263 | Hs.73172 | GFI1 |
| 3114 | 217914_at | 4566.87 | 43.53 | 1.13 | 1.23 | NM_017901 | Hs.26440 | TPCN1 |
| 3115 | 218113_at | 4567.58 | 43.54 | 1.15 | 1.25 | NM_013390 | Hs.160417 | TMEM2 |
| 3116 | 209374_s_at | 4567.86 | 43.54 | 1.06 | 1.10 | BC001872 | Hs.439852 | IGHM |
| 3117 | 212159_x_at | 4568.16 | 43.54 | 1.15 | 1.26 | AI125280 | Hs.19121 | AP2A2 |
| 3118 | 204520_x_at | 4568.22 | 43.53 | 1.15 | 1.25 | NM_014577 | Hs.127950 | BRD1 |
| 3119 | 209769_s_at | 4569.65 | 43.56 | 1.15 | 1.25 | L20860 | Hs.512000 | GP1BB |
| 3120 | 202192_s_at | 4569.86 | 43.56 | 1.13 | 1.22 | NM_005890 | Hs.226133 | GAS7 |
| 3121 | 204643_s_at | 4573.57 | 43.69 | 1.15 | 1.26 | NM_006375 | Hs.155185 | COVA1 |
| 3122 | 203659_s_at | 4573.93 | 43.69 | 1.15 | 1.25 | NM_005798 | Hs.436922 | RFP2 |
| 3123 | 221198_at | 4574.02 | 43.68 | 1.13 | 1.23 | NM_021920 | — | SCT |
| 3124 | 218039_at | 4576.57 | 43.76 | 1.15 | 1.25 | NM_016359 | Hs.279905 | NUSAP1 |
| 3125 | 203543_s_at | 4576.76 | 43.76 | 1.13 | 1.23 | NM_001206 | Hs.150557 | BTEB1 |
| 3126 | 205349_at | 4577.47 | 43.77 | 1.06 | 1.09 | NM_002068 | Hs.73797 | GNA15 |
| 3127 | 200844_s_at | 4578.36 | 43.79 | 1.11 | 1.19 | BE869583 | Hs.120 | PRDX6 |
| 3128 | 210298_x_at | 4578.99 | 43.80 | 1.13 | 1.23 | AF098518 | Hs.421383 | FHL1 |
| 3129 | 213274_s_at | 4582.64 | 43.92 | 1.09 | 1.16 | AA020826 | Hs.135226 | CTSB |
| 3130 | 215045_at | 4584.53 | 43.99 | 1.15 | 1.25 | BC004145 | Hs.26047 | TNRC4 |
| 3131 | 215772_x_at | 4585.20 | 44.00 | 1.12 | 1.20 | AL050226 | — | — |
| 3132 | 212280_x_at | 4585.55 | 44.00 | 1.13 | 1.22 | AA532726 | Hs.283610 | APG4B |
| 3133 | 216473_x_at | 4585.92 | 43.99 | 1.12 | 1.21 | D38024 | — | — |
| 3134 | 221953_s_at | 4586.39 | 44.00 | 1.14 | 1.25 | W45551 | Hs.5215 | ITGB4BP |
| 3135 | 203712_at | 4586.62 | 43.99 | 1.15 | 1.27 | NM_014878 | Hs.443866 | KIAA0020 |
| 3136 | 221687_s_at | 4587.73 | 44.03 | 1.15 | 1.25 | BC000122 | Hs.438972 | C9orf28 |
| 3137 | 201393_s_at | 4587.75 | 44.01 | 1.11 | 1.18 | NM_000876 | Hs.76473 | IGF2R |
| 3138 | 200850_s_at | 4588.97 | 44.05 | 1.15 | 1.26 | NM_006621 | Hs.4113 | AHCYL1 |
| 3139 | 212880_at | 4589.25 | 44.04 | 1.15 | 1.26 | AB011113 | Hs.428344 | WDR7 |
| 3140 | 206016_at | 4589.74 | 44.05 | 1.12 | 1.20 | NM_014008 | Hs.26333 | CXorf37 |
| 3141 | 217835_x_at | 4590.58 | 44.07 | 1.14 | 1.24 | NM_018840 | Hs.184062 | C20orf24 |
| 3142 | 210660_at | 4591.13 | 44.08 | 1.11 | 1.19 | AF025529 | — | LILRA1 |
| 3143 | 210322_x_at | 4591.73 | 44.09 | 1.15 | 1.25 | AF000995 | Hs.115277 | UTY |
| 3144 | 201029_s_at | 4592.15 | 44.09 | 1.15 | 1.26 | NM_002414 | Hs.283477 | CD99 |
| 3145 | 203384_s_at | 4592.17 | 44.08 | 1.14 | 1.24 | NM_002077 | Hs.101916 | GOLGA1 |
| 3146 | 205726_at | 4592.94 | 44.09 | 1.13 | 1.23 | NM_006729 | — | DIAPH2 |
| 3147 | 210116_at | 4594.35 | 44.13 | 1.03 | 1.05 | AF072930 | Hs.349094 | SH2D1A |
| 3148 | 216345_at | 4594.87 | 44.14 | 1.14 | 1.24 | AB020720 | Hs.65135 | KIAA0913 |
| 3149 | 205518_s_at | 4595.43 | 44.15 | 1.11 | 1.19 | NM_003570 | — | CMAH |
| 3150 | 213016_at | 4596.69 | 44.18 | 1.12 | 1.20 | AA573805 | Hs.35380 | BBX |
| 3151 | 214962_s_at | 4597.30 | 44.20 | 1.15 | 1.26 | AK026236 | Hs.22559 | NUP160 |
| 3152 | 203200_s_at | 4597.41 | 44.19 | 1.14 | 1.24 | NM_024010 | Hs.153792 | MTRR |
| 3153 | 214129_at | 4598.58 | 44.22 | 1.15 | 1.26 | AI821791 | Hs.512356 | — |
| 3154 | 202131_s_at | 4600.83 | 44.31 | 1.14 | 1.24 | NM_003831 | Hs.209061 | RIOK3 |
| 3155 | 219690_at | 4601.08 | 44.31 | 1.12 | 1.20 | NM_024660 | Hs.352548 | FLJ22573 |
| 3156 | 212535_at | 4602.36 | 44.35 | 1.15 | 1.26 | AA142929 | Hs.415033 | MEF2A |
| 3157 | 208353_x_at | 4602.49 | 44.34 | 1.13 | 1.21 | NM_020480 | — | — |
| 3158 | 209019_s_at | 4603.28 | 44.35 | 1.14 | 1.24 | AF316873 | Hs.389171 | PINK1 |
| 3159 | 206546_at | 4605.27 | 44.43 | 1.16 | 1.27 | NM_014258 | Hs.202676 | SYCP2 |
| 3160 | 204959_at | 4605.46 | 44.43 | 1.12 | 1.21 | NM_002432 | Hs.153837 | MNDA |
| 3161 | 203932_at | 4605.83 | 44.43 | 1.14 | 1.24 | NM_002118 | Hs.1162 | HLA-DMB |
| 3162 | 214674_at | 4605.84 | 44.41 | 1.14 | 1.23 | AW451502 | Hs.255596 | USP19 |
| 3163 | 204497_at | 4607.62 | 44.47 | 1.12 | 1.21 | AB011092 | Hs.20196 | ADCY9 |
| 3164 | 220038_at | 4607.76 | 44.46 | 1.13 | 1.21 | NM_013257 | Hs.380877 | SGKL |
| 3165 | 202568_s_at | 4607.86 | 44.45 | 1.15 | 1.26 | AI745639 | Hs.437625 | MARK3 |
| 3166 | 207654_x_at | 4608.69 | 44.47 | 1.11 | 1.19 | NM_001938 | Hs.348418 | DR1 |
| 3167 | 210144_at | 4610.23 | 44.52 | 1.14 | 1.25 | AK000851 | Hs.505862 | C22orf4 |
| 3168 | 204312_x_at | 4611.24 | 44.54 | 1.16 | 1.27 | AI655737 | Hs.22315 | CREB1 |
| 3169 | 200650_s_at | 4613.33 | 44.61 | 1.15 | 1.26 | NM_005566 | Hs.2795 | LDHA |
| 3170 | 210317_s_at | 4615.11 | 44.66 | 1.13 | 1.22 | U28936 | Hs.79474 | YWHAE |
| 3171 | 201999_s_at | 4616.96 | 44.72 | 1.13 | 1.23 | NM_006519 | Hs.266940 | TCTEL1 |
| 3172 | 220674_at | 4617.55 | 44.73 | 1.15 | 1.25 | NM_024916 | Hs.528682 | FLJ22814 |
| 3173 | 221932_s_at | 4617.88 | 44.73 | 1.15 | 1.25 | AA133341 | Hs.294083 | C14orf87 |
| 3174 | 217322_x_at | 4618.09 | 44.72 | 1.14 | 1.25 | AL024509 | — | — |
| 3175 | 209450_at | 4618.73 | 44.73 | 1.15 | 1.25 | AB050442 | Hs.413652 | OSGEP |
| 3176 | 200911_s_at | 4619.30 | 44.74 | 1.16 | 1.27 | NM_006283 | Hs.279245 | TACC1 |
| 3177 | 203232_s_at | 4619.59 | 44.73 | 1.11 | 1.19 | NM_000332 | Hs.434961 | ATXN1 |
| 3178 | 214093_s_at | 4620.11 | 44.74 | 1.09 | 1.15 | AA156865 | Hs.118962 | FUBP1 |
| 3179 | 200891_s_at | 4621.14 | 44.77 | 1.14 | 1.23 | NM_003144 | Hs.250773 | SSR1 |
| 3180 | 214211_at | 4622.19 | 44.79 | 1.09 | 1.15 | AA083483 | Hs.446345 | FTH1 |
| 3181 | 208857_s_at | 4622.67 | 44.80 | 1.13 | 1.23 | M93008 | Hs.79137 | PCMT1 |
| 3182 | 210249_s_at | 4622.93 | 44.80 | 1.14 | 1.25 | U59302 | Hs.386092 | NCOA1 |
| 3183 | 204442_x_at | 4623.36 | 44.81 | 1.14 | 1.24 | NM_003573 | Hs.85087 | LTBP4 |
| 3184 | 212761_at | 4625.17 | 44.87 | 1.09 | 1.15 | AI949687 | Hs.214039 | TCF7L2 |
| 3185 | 222127_s_at | 4625.76 | 44.88 | 1.15 | 1.26 | AK023461 | Hs.221377 | SEC3L1 |
| 3186 | 222348_at | 4626.57 | 44.90 | 1.13 | 1.21 | AW971134 | — | — |
| 3187 | 200662_s_at | 4627.46 | 44.92 | 1.16 | 1.28 | NM_014765 | Hs.75187 | TOMM20 |
| 3188 | 218606_at | 4628.21 | 44.94 | 1.14 | 1.24 | NM_017740 | Hs.9725 | ZDHHC7 |
| 3189 | 218608_at | 4628.85 | 44.95 | 1.15 | 1.27 | NM_022089 | Hs.128866 | HSA9947 |
| 3190 | 210616_s_at | 4630.11 | 44.99 | 1.15 | 1.25 | AB020712 | Hs.436549 | SEC31L1 |
| 3191 | 203600_s_at | 4631.66 | 45.03 | 1.15 | 1.26 | NM_003704 | Hs.325987 | C4orf8 |
| 3192 | 217808_s_at | 4631.87 | 45.03 | 1.13 | 1.23 | NM_024117 | Hs.324178 | MAPKAP1 |
| 3193 | 205711_x_at | 4632.92 | 45.05 | 1.13 | 1.22 | NM_005174 | — | — |
| 3194 | 201861_s_at | 4635.54 | 45.14 | 1.14 | 1.24 | BF965566 | Hs.512387 | LRRFIP1 |
| 3195 | 202875_s_at | 4637.73 | 45.22 | 1.11 | 1.19 | BE397715 | Hs.93728 | PBX2 |
| 3196 | 213128_s_at | 4637.77 | 45.20 | 1.16 | 1.27 | AA527499 | Hs.180686 | UBE3A |
| 3197 | 219116_s_at | 4637.88 | 45.19 | 1.14 | 1.24 | NM_018185 | Hs.23618 | C13orf17 |
| 3198 | 219947_at | 4638.80 | 45.22 | 1.15 | 1.26 | NM_016184 | Hs.115515 | CLECSF6 |
| 3199 | 202524_s_at | 4639.26 | 45.22 | 1.13 | 1.22 | NM_014767 | Hs.436193 | SPOCK2 |
| 3200 | 200964_at | 4639.27 | 45.20 | 1.14 | 1.24 | NM_003334 | Hs.406693 | UBE1 |
| 3201 | 217422_s_at | 4639.33 | 45.19 | 1.15 | 1.26 | X52785 | Hs.262150 | CD22 |
| 3202 | 203605_at | 4639.66 | 45.19 | 1.14 | 1.24 | NM_003136 | Hs.49346 | SRP54 |
| 3203 | 212054_x_at | 4639.99 | 45.19 | 1.16 | 1.27 | AK026096 | Hs.155629 | KIAA0676 |
| 3204 | 215109_at | 4642.34 | 45.28 | 1.14 | 1.24 | R02172 | Hs.531342 | KIAA0492 |
| 3205 | 213300_at | 4642.95 | 45.29 | 1.13 | 1.22 | AW168132 | Hs.370671 | KIAA0404 |
| 3206 | 205811_at | 4643.05 | 45.28 | 1.15 | 1.26 | NM_007215 | Hs.437009 | POLG2 |
| 3207 | 213891_s_at | 4643.63 | 45.29 | 1.11 | 1.19 | AI927067 | — | — |
| 3208 | 201055_s_at | 4646.26 | 45.39 | 1.14 | 1.23 | NM_006805 | Hs.96996 | HNRPA0 |
| 3209 | 217602_at | 4646.40 | 45.38 | 1.07 | 1.12 | AI191118 | — | — |
| 3210 | 216050_at | 4646.56 | 45.37 | 0.91 | 0.86 | AK024584 | Hs.406847 | — |
| 3211 | 209814_at | 4647.26 | 45.38 | 1.15 | 1.26 | BC004421 | Hs.120766 | ZNF330 |
| 3212 | 217815_at | 4647.79 | 45.38 | 1.16 | 1.27 | NM_007192 | Hs.271225 | SUPT16H |
| 3213 | 218048_at | 4648.01 | 45.38 | 1.16 | 1.28 | NM_012071 | Hs.380403 | COMMD3 |
| 3214 | 208315_x_at | 4648.11 | 45.37 | 1.15 | 1.26 | NM_003300 | Hs.297660 | TRAF3 |
| 3215 | 205922_at | 4648.70 | 45.38 | 1.07 | 1.12 | NM_004665 | — | — |
| 3216 | 202604_x_at | 4649.12 | 45.38 | 1.14 | 1.25 | NM_001110 | Hs.172028 | ADAM10 |
| 3217 | 200065_s_at | 4649.16 | 45.37 | 1.15 | 1.27 | AF052179 | Hs.286221 | ARF1 |
| 3218 | 207974_s_at | 4650.31 | 45.40 | 1.08 | 1.14 | NM_006930 | Hs.171626 | SKP1A |
| 3219 | 209274_s_at | 4652.75 | 45.49 | 1.15 | 1.27 | BC002675 | — | — |
| 3220 | 210094_s_at | 4654.57 | 45.55 | 1.07 | 1.11 | AF196186 | Hs.72249 | PARD3 |
| 3221 | 201563_at | 4657.01 | 45.64 | 1.13 | 1.22 | L29008 | Hs.878 | SORD |
| 3222 | 210840_s_at | 4657.98 | 45.66 | 1.12 | 1.20 | D29640 | Hs.1742 | IQGAP1 |
| 3223 | 58780_s_at | 4658.92 | 45.68 | 1.09 | 1.15 | R42449 | Hs.22451 | FLJ10357 |
| 3224 | 201086_x_at | 4659.49 | 45.69 | 1.16 | 1.28 | NM_003103 | Hs.430541 | SON |
| 3225 | 212434_at | 4660.42 | 45.71 | 1.13 | 1.22 | AI984421 | Hs.151903 | GRPEL1 |
| 3226 | 215966_x_at | 4660.68 | 45.71 | 1.13 | 1.22 | AA292874 | — | — |
| 3227 | 204489_s_at | 4660.75 | 45.70 | 1.14 | 1.24 | NM_000610 | Hs.306278 | CD44 |
| 3228 | 213037_x_at | 4661.13 | 45.70 | 1.15 | 1.26 | AJ132258 | — | — |
| 3229 | 216379_x_at | 4661.75 | 45.71 | 0.98 | 0.96 | AK000168 | Hs.375108 | CD24 |
| 3230 | 201041_s_at | 4661.78 | 45.70 | 1.02 | 1.04 | NM_004417 | Hs.171695 | DUSP1 |
| 3231 | 203035_s_at | 4661.84 | 45.69 | 1.16 | 1.27 | NM_006099 | Hs.435761 | PIAS3 |
| 3232 | 209188_x_at | 4661.99 | 45.68 | 1.12 | 1.20 | BC002809 | Hs.348418 | DR1 |
| 3233 | 221185_s_at | 4662.25 | 45.68 | 1.13 | 1.22 | NM_025111 | Hs.512778 | DKFZp434B227 |
| 3234 | 209107_x_at | 4663.78 | 45.73 | 1.14 | 1.24 | U19179 | Hs.386092 | NCOA1 |
| 3235 | 212204_at | 4664.50 | 45.74 | 1.16 | 1.27 | AL049944 | Hs.200692 | DKFZP564G2022 |
| 3236 | 213088_s_at | 4664.98 | 45.75 | 1.15 | 1.27 | BE551340 | Hs.400676 | DNAJC9 |
| 3237 | 221525_at | 4665.46 | 45.75 | 1.13 | 1.22 | AL136572 | Hs.77978 | DKFZp761I2123 |
| 3238 | 209175_at | 4665.94 | 45.75 | 1.12 | 1.20 | AK001135 | Hs.300208 | SEC23IP |
| 3239 | 201532_at | 4666.93 | 45.79 | 1.10 | 1.16 | NM_002788 | Hs.246240 | PSMA3 |
| 3240 | 201243_s_at | 4667.24 | 45.78 | 1.09 | 1.16 | NM_001677 | Hs.78629 | ATP1B1 |
| 3241 | 202433_at | 4667.27 | 45.77 | 1.16 | 1.28 | NM_005827 | Hs.154073 | SLC35B1 |
| 3242 | 209367_at | 4667.60 | 45.77 | 1.09 | 1.15 | AB002559 | Hs.379204 | STXBP2 |
| 3243 | 202776_at | 4667.86 | 45.77 | 1.13 | 1.23 | NM_014597 | Hs.85769 | ERBP |
| 3244 | 209508_x_at | 4668.51 | 45.79 | 1.13 | 1.21 | AF005774 | Hs.355724 | CFLAR |
| 3245 | 218543_s_at | 4668.86 | 45.79 | 1.07 | 1.13 | NM_022750 | Hs.12646 | ZC3HDC1 |
| 3246 | 201620_at | 4669.41 | 45.79 | 1.15 | 1.26 | NM_003791 | Hs.75890 | MBTPS1 |
| 3247 | 200929_at | 4669.42 | 45.78 | 1.14 | 1.25 | NM_006827 | Hs.74137 | TMP21 |
| 3248 | 202317_s_at | 4670.01 | 45.79 | 1.15 | 1.26 | NM_006048 | Hs.386404 | UBE4B |
| 3249 | 212194_s_at | 4670.14 | 45.78 | 1.11 | 1.19 | AI418892 | Hs.79305 | TM9SF4 |
| 3250 | 213049_at | 4670.39 | 45.77 | 1.13 | 1.22 | BG436400 | Hs.167031 | GARNL1 |
| 3251 | 212961_x_at | 4671.16 | 45.79 | 1.16 | 1.27 | L43578 | Hs.425383 | CXorf40 |
| 3252 | 208806_at | 4672.31 | 45.83 | 1.11 | 1.19 | BE379542 | Hs.25601 | CHD3 |
| 3253 | 208407_s_at | 4673.88 | 45.88 | 1.16 | 1.27 | NM_001331 | Hs.166011 | CTNND1 |
| 3254 | 204333_s_at | 4674.05 | 45.87 | 1.11 | 1.19 | NM_000027 | — | — |
| 3255 | 215850_s_at | 4674.82 | 45.89 | 1.15 | 1.26 | AK022209 | Hs.83916 | NDUFA5 |
| 3256 | 219443_at | 4674.95 | 45.88 | 1.14 | 1.25 | NM_017714 | Hs.88367 | C20orf13 |
| 3257 | 204324_s_at | 4675.06 | 45.87 | 1.09 | 1.15 | NM_014498 | Hs.143600 | GOLPH4 |
| 3258 | 200885_at | 4676.30 | 45.91 | 1.08 | 1.13 | NM_005167 | Hs.179735 | RHOC |
| 3259 | 211764_s_at | 4676.61 | 45.90 | 1.08 | 1.13 | BC005980 | Hs.129683 | UBE2D1 |
| 3260 | 221699_s_at | 4679.36 | 46.00 | 1.16 | 1.27 | AF334103 | Hs.396766 | DDX50 |
| 3261 | 217943_s_at | 4680.52 | 46.04 | 1.14 | 1.24 | NM_018067 | Hs.356096 | FLJ10350 |
| 3262 | 213292_s_at | 4680.94 | 46.04 | 1.15 | 1.26 | AA908770 | Hs.269881 | SNX13 |
| 3263 | 219951_s_at | 4681.08 | 46.03 | 1.16 | 1.28 | NM_018152 | Hs.128766 | C20orf12 |
| 3264 | 220917_s_at | 4681.65 | 46.04 | 1.14 | 1.24 | NM_025132 | Hs.438482 | WDR19 |
| 3265 | 217516_x_at | 4682.03 | 46.04 | 1.13 | 1.23 | BG149428 | Hs.326730 | ARVCF |
| 3266 | 215024_at | 4684.22 | 46.12 | 1.14 | 1.24 | AK000993 | Hs.274128 | — |
| 3267 | 204928_s_at | 4686.25 | 46.18 | 1.14 | 1.24 | NM_019848 | Hs.72980 | SLC10A3 |
| 3268 | 203409_at | 4686.41 | 46.17 | 1.10 | 1.18 | NM_000107 | Hs.446564 | DDB2 |
| 3269 | 203971_at | 4687.37 | 46.20 | 1.12 | 1.20 | NM_001859 | Hs.414471 | SLC31A1 |
| 3270 | 206277_at | 4688.04 | 46.22 | 1.12 | 1.20 | NM_002564 | Hs.339 | P2RY2 |
| 3271 | 221771_s_at | 4689.55 | 46.26 | 1.14 | 1.25 | BC003542 | Hs.411347 | HSMPP8 |
| 3272 | 209282_at | 4690.31 | 46.28 | 1.15 | 1.25 | AF309082 | Hs.205431 | PRKD2 |
| 3273 | 204878_s_at | 4691.05 | 46.29 | 1.13 | 1.23 | NM_004783 | Hs.291623 | TAO1 |
| 3274 | 218768_at | 4691.67 | 46.31 | 1.16 | 1.28 | NM_020401 | Hs.355598 | NUP107 |
| 3275 | 214500_at | 4691.86 | 46.30 | 1.12 | 1.21 | AF044286 | Hs.75258 | H2AFY |
| 3276 | 213280_at | 4692.84 | 46.33 | 1.12 | 1.20 | AK000478 | Hs.499659 | GARNL4 |
| 3277 | 218058_at | 4693.15 | 46.33 | 1.16 | 1.28 | NM_014593 | Hs.180933 | CXXC1 |
| 3278 | 209300_s_at | 4693.29 | 46.32 | 1.15 | 1.26 | BC002888 | Hs.527839 | DKFZP566B183 |
| 3279 | 215273_s_at | 4693.45 | 46.31 | 1.13 | 1.23 | AK024982 | Hs.386390 | TADA3L |
| 3280 | 206072_at | 4693.64 | 46.30 | 1.14 | 1.24 | NM_003353 | Hs.134932 | UCN |
| 3281 | 217672_x_at | 4693.66 | 46.29 | 1.12 | 1.21 | BF114906 | — | — |
| 3282 | 201941_at | 4694.21 | 46.30 | 1.14 | 1.24 | BE349147 | Hs.5057 | CPD |
| 3283 | 209578_s_at | 4697.20 | 46.40 | 1.13 | 1.23 | BC000626 | Hs.22982 | POFUT2 |
| 3284 | 222290_at | 4698.96 | 46.46 | 1.12 | 1.20 | AA731709 | Hs.133517 | OR2A20P |
| 3285 | 211230_s_at | 4699.91 | 46.48 | 1.14 | 1.25 | U57843 | Hs.426967 | PIK3CD |
| 3286 | 212196_at | 4701.20 | 46.53 | 1.07 | 1.12 | AW242916 | Hs.71968 | IL6ST |
| 3287 | 209390_at | 4701.68 | 46.53 | 1.14 | 1.23 | AF013168 | Hs.69429 | TSC1 |
| 3288 | 213268_at | 4701.97 | 46.53 | 1.11 | 1.18 | Z98884 | Hs.253254 | CAMTA1 |
| 3289 | 222129_at | 4702.08 | 46.52 | 1.14 | 1.25 | AK026155 | Hs.22412 | C2orf17 |
| 3290 | 207541_s_at | 4705.02 | 46.62 | 1.15 | 1.26 | NM_002685 | Hs.321234 | EXOSC10 |
| 3291 | 201127_s_at | 4705.72 | 46.63 | 1.15 | 1.26 | AI971281 | Hs.387567 | ACLY |
| 3292 | 213164_at | 4706.53 | 46.66 | 1.16 | 1.27 | AI867198 | — | SLC5A3 |
| 3293 | 203655_at | 4707.52 | 46.69 | 1.14 | 1.25 | NM_006297 | Hs.98493 | XRCC1 |
| 3294 | 219974_x_at | 4708.99 | 46.72 | 1.15 | 1.25 | NM_018479 | Hs.437091 | ECHDC1 |
| 3295 | 202126_at | 4710.03 | 46.75 | 1.07 | 1.11 | AA156948 | Hs.198891 | PRPF4B |
| 3296 | 201572_x_at | 4710.45 | 46.75 | 1.15 | 1.26 | NM_001921 | Hs.76894 | DCTD |
| 3297 | 222006_at | 4711.03 | 46.76 | 1.14 | 1.24 | AI359368 | Hs.1420 | FGFR3 |
| 3298 | 203544_s_at | 4712.12 | 46.78 | 1.15 | 1.25 | NM_003473 | Hs.441498 | STAM |
| 3299 | 208989_s_at | 4712.89 | 46.80 | 1.13 | 1.23 | AF179221 | Hs.219614 | FBXL11 |
| 3300 | 202393_s_at | 4714.33 | 46.84 | 0.96 | 0.93 | NM_005655 | Hs.82173 | TIEG |
| 3301 | 209371_s_at | 4714.66 | 46.84 | 1.11 | 1.19 | AB000462 | Hs.167679 | SH3BP2 |
| 3302 | 212518_at | 4714.83 | 46.83 | 1.12 | 1.20 | AB011161 | Hs.282177 | PIP5K1C |
| 3303 | 202188_at | 4716.25 | 46.88 | 1.14 | 1.24 | NM_014669 | Hs.295014 | NUP93 |
| 3304 | 215599_at | 4716.33 | 46.86 | 1.02 | 1.03 | X83300 | — | SMA4 |
| 3305 | 219555_s_at | 4717.46 | 46.90 | 1.15 | 1.25 | NM_018455 | Hs.283532 | BM039 |
| 3306 | 208805_at | 4717.63 | 46.89 | 1.14 | 1.23 | BC002979 | Hs.374499 | PSMA6 |
| 3307 | 220261_s_at | 4718.87 | 46.92 | 1.14 | 1.25 | NM_018106 | Hs.5268 | ZDHHC4 |
| 3308 | 214213_x_at | 4719.10 | 46.92 | 1.14 | 1.23 | AA063189 | Hs.436441 | LMNA |
| 3309 | 208974_x_at | 4719.61 | 46.93 | 1.12 | 1.21 | BC003572 | Hs.439683 | KPNB1 |
| 3310 | 213979_s_at | 4720.74 | 46.96 | 1.01 | 1.01 | BF984434 | — | — |
| 3311 | 218617_at | 4720.90 | 46.96 | 1.15 | 1.26 | NM_017646 | Hs.356554 | TRIT1 |
| 3312 | 202759_s_at | 4721.67 | 46.98 | 1.14 | 1.23 | BE879367 | — | AKAP2 |
| 3313 | 205758_at | 4722.23 | 46.99 | 0.99 | 0.99 | AW006735 | Hs.85258 | CD8A |
| 3314 | 214170_x_at | 4722.25 | 46.97 | 1.15 | 1.26 | AA669797 | Hs.391168 | FH |
| 3315 | 218019_s_at | 4722.86 | 46.98 | 1.12 | 1.21 | NM_021941 | Hs.284491 | PDXK |
| 3316 | 201593_s_at | 4722.90 | 46.97 | 1.11 | 1.18 | AV716798 | Hs.368598 | LEREPO4 |
| 3317 | 208671_at | 4724.07 | 47.01 | 1.11 | 1.19 | AF164794 | Hs.146668 | TDE2 |
| 3318 | 218306_s_at | 4724.40 | 47.01 | 1.16 | 1.27 | NM_003922 | Hs.133411 | HERC1 |
| 3319 | 201102_s_at | 4724.40 | 46.99 | 1.15 | 1.26 | NM_002626 | Hs.368741 | PFKL |
| 3320 | 203814_s_at | 4724.58 | 46.98 | 0.99 | 0.98 | NM_000904 | — | NQO2 |
| 3321 | 207707_s_at | 4725.92 | 47.02 | 1.15 | 1.26 | NM_030673 | Hs.227949 | SEC13L1 |
| 3322 | 201685_s_at | 4725.98 | 47.01 | 1.14 | 1.24 | NM_014828 | Hs.194035 | C14orf92 |
| 3323 | 203274_at | 4726.67 | 47.02 | 1.14 | 1.24 | NM_012151 | Hs.387463 | F8A |
| 3324 | 215388_s_at | 4727.32 | 47.04 | 1.13 | 1.21 | X56210 | Hs.296941 | CFHL1 |
| 3325 | 218088_s_at | 4730.04 | 47.14 | 1.14 | 1.23 | NM_022157 | Hs.110950 | RRAGC |
| 3326 | 203143_s_at | 4730.82 | 47.15 | 1.13 | 1.22 | T79953 | Hs.368916 | KIAA0040 |
| 3327 | 204409_s_at | 4730.91 | 47.14 | 1.14 | 1.24 | BC005248 | Hs.461178 | EIF1AY |
| 3328 | 217860_at | 4730.94 | 47.13 | 1.15 | 1.25 | NM_004544 | Hs.198271 | NDUFA10 |
| 3329 | 201513_at | 4731.04 | 47.12 | 1.14 | 1.24 | AI659180 | Hs.75066 | TSN |
| 3330 | 215731_s_at | 4731.08 | 47.11 | 1.14 | 1.24 | X98258 | Hs.445084 | MPHOSPH9 |
| 3331 | 210145_at | 4731.09 | 47.09 | 1.14 | 1.25 | M68874 | Hs.211587 | PLA2G4A |
| 3332 | 212863_x_at | 4731.40 | 47.09 | 1.10 | 1.17 | BF337195 | Hs.196083 | CTBP1 |
| 3333 | 200695_at | 4731.50 | 47.08 | 1.14 | 1.24 | NM_014225 | Hs.173902 | PPP2R1A |
| 3334 | 218014_at | 4731.59 | 47.07 | 1.14 | 1.24 | NM_024844 | Hs.184352 | PCNT1 |
| 3335 | 203166_at | 4733.20 | 47.13 | 1.12 | 1.20 | NM_006324 | Hs.433402 | CFDP1 |
| 3336 | 201054_at | 4733.79 | 47.14 | 1.07 | 1.12 | BE966599 | Hs.96996 | HNRPA0 |
| 3337 | 204299_at | 4734.55 | 47.16 | 1.16 | 1.27 | NM_021993 | Hs.3530 | FUSIP1 |
| 3338 | 204139_x_at | 4735.94 | 47.20 | 1.11 | 1.18 | NM_003422 | Hs.458361 | ZNF42 |
| 3339 | 210772_at | 473704 | 47.23 | 1.12 | 1.21 | M88107 | Hs.99855 | FPRL1 |
| 3340 | 206268_at | 4738.02 | 47.25 | 1.08 | 1.14 | NM_020997 | Hs.278239 | LEFTB |
| 3341 | 205047_s_at | 4738.22 | 47.25 | 1.11 | 1.19 | NM_001673 | Hs.446546 | ASNS |
| 3342 | 220990_s_at | 4739.33 | 47.28 | 1.12 | 1.21 | NM_030938 | Hs.166254 | VMP1 |
| 3343 | 203581_at | 4739.92 | 47.29 | 1.05 | 1.09 | BC002438 | Hs.296169 | RAB4A |
| 3344 | 212841_s_at | 4741.56 | 47.35 | 1.10 | 1.17 | AI692180 | Hs.12953 | PPFIBP2 |
| 3345 | 208866_at | 4741.59 | 47.33 | 1.11 | 1.19 | BF510713 | Hs.318381 | CSNK1A1 |
| 3346 | 204905_s_at | 4741.84 | 47.33 | 1.09 | 1.15 | NM_004280 | Hs.88977 | EEF1E1 |
| 3347 | 218793_s_at | 4742.91 | 47.37 | 1.02 | 1.03 | NM_006746 | Hs.109655 | SCML1 |
| 3348 | 221858_at | 4743.18 | 47.36 | 1.13 | 1.22 | N34407 | Hs.100960 | TBC1D12 |
| 3349 | 209652_s_at | 4743.35 | 47.36 | 1.14 | 1.24 | BC001422 | Hs.252820 | PGF |
| 3350 | 213178_s_at | 4743.62 | 47.35 | 1.10 | 1.17 | AB028989 | Hs.511943 | MAPK8IP3 |
| 3351 | 201458_s_at | 4744.01 | 47.35 | 1.15 | 1.26 | NM_004725 | Hs.418533 | BUB3 |
| 3352 | 218521_s_at | 4744.50 | 47.36 | 1.08 | 1.13 | NM_018299 | Hs.376816 | FLJ11011 |
| 3353 | 205297_s_at | 4747.39 | 47.46 | 1.04 | 1.06 | NM_000626 | Hs.89575 | CD79B |
| 3354 | 216166_at | 4749.47 | 47.53 | 1.10 | 1.17 | AK024909 | Hs.306724 | — |
| 3355 | 208794_s_at | 4749.91 | 47.54 | 1.12 | 1.20 | D26156 | Hs.78202 | SMARCA4 |
| 3356 | 222032_s_at | 4749.94 | 47.52 | 1.14 | 1.23 | BF591638 | — | — |
| 3357 | 206550_s_at | 4750.15 | 47.52 | 1.14 | 1.24 | NM_004298 | Hs.23255 | NUP155 |
| 3358 | 201923_at | 4750.27 | 47.51 | 1.01 | 1.01 | NM_006406 | Hs.83383 | PRDX4 |
| 3359 | 204669_s_at | 4751.51 | 47.54 | 1.11 | 1.19 | NM_007219 | Hs.30524 | RNF24 |
| 3360 | 213074_at | 4754.08 | 47.62 | 1.13 | 1.22 | BG545769 | Hs.511817 | IRAK1BP1 |
| 3361 | 217549_at | 4754.49 | 47.63 | 1.15 | 1.26 | AW574933 | — | — |
| 3362 | 215626_at | 4754.61 | 47.62 | 1.14 | 1.24 | AU144887 | Hs.301800 | — |
| 3363 | 215401_at | 4755.10 | 47.63 | 1.12 | 1.21 | AU147698 | Hs.507633 | — |
| 3364 | 211703_s_at | 4755.55 | 47.63 | 1.11 | 1.19 | AF353990 | Hs.276876 | BBP |
| 3365 | 207434_s_at | 4756.75 | 47.66 | 1.11 | 1.18 | NM_021603 | Hs.413137 | FXYD2 |
| 3366 | 201064_s_at | 4756.99 | 47.66 | 1.16 | 1.27 | NM_003819 | Hs.169900 | PABPC4 |
| 3367 | 219972_s_at | 4757.39 | 47.66 | 1.12 | 1.21 | NM_022495 | Hs.413671 | C14orf135 |
| 3368 | 200991_s_at | 4759.57 | 47.75 | 1.13 | 1.23 | NM_014748 | — | — |
| 3369 | 208901_s_at | 4760.88 | 47.79 | 1.15 | 1.25 | J03250 | Hs.253536 | TOP1 |
| 3370 | 203165_s_at | 4760.98 | 47.77 | 1.15 | 1.25 | NM_004733 | Hs.285176 | SLC33A1 |
| 3371 | 209421_at | 4761.49 | 47.78 | 1.14 | 1.24 | U04045 | Hs.440394 | MSH2 |
| 3372 | 206328_at | 4761.60 | 47.77 | 1.12 | 1.21 | NM_004933 | Hs.148090 | CDH15 |
| 3373 | 222212_s_at | 4763.93 | 47.85 | 1.12 | 1.21 | AK001105 | Hs.285976 | LASS2 |
| 3374 | 212712_at | 4764.39 | 47.85 | 1.14 | 1.25 | BE222901 | Hs.380639 | CAMSAP1 |
| 3375 | 216813_at | 4766.39 | 47.93 | 1.11 | 1.19 | AL512728 | — | — |
| 3376 | 217968_at | 4767.27 | 47.95 | 1.13 | 1.23 | NM_003310 | Hs.4992 | TSSC1 |
| 3377 | 212306_at | 4767.63 | 47.95 | 1.16 | 1.28 | AI741784 | Hs.108614 | CLASP2 |
| 3378 | 222209_s_at | 4768.15 | 47.96 | 1.14 | 1.23 | AK000684 | Hs.183887 | FLJ22104 |
| 3379 | 202402_s_at | 4768.62 | 47.97 | 1.13 | 1.23 | NM_001751 | Hs.304134 | CARS |
| 3380 | 211938_at | 4769.58 | 48.00 | 1.15 | 1.25 | BF247371 | Hs.93379 | EIF4B |
| 3381 | 207556_s_at | 4770.07 | 48.00 | 1.09 | 1.15 | NM_003646 | Hs.277445 | DGKZ |
| 3382 | 209880_s_at | 4772.36 | 48.07 | 1.14 | 1.24 | U02297 | Hs.423077 | SELPLG |
| 3383 | 202591_s_at | 4773.19 | 48.09 | 1.14 | 1.23 | NM_003143 | Hs.923 | SSBP1 |
| 3384 | 205213_at | 4774.30 | 48.12 | 1.13 | 1.22 | NM_014716 | Hs.337242 | CENTB1 |
| 3385 | 200901_s_at | 4774.39 | 48.11 | 1.15 | 1.26 | NM_002355 | Hs.134084 | M6PR |
| 3386 | 212065_s_at | 4775.69 | 48.15 | 1.00 | 1.00 | AW502434 | Hs.507665 | USP34 |
| 3387 | 215460_x_at | 4775.99 | 48.15 | 1.13 | 1.23 | AL080149 | Hs.127950 | BRD1 |
| 3388 | 202916_s_at | 4776.49 | 48.15 | 1.15 | 1.25 | NM_014864 | Hs.5737 | FAM20B |
| 3389 | 207108_s_at | 4777.21 | 48.17 | 1.15 | 1.26 | NM_015384 | Hs.225767 | NIPBL |
| 3390 | 201967_at | 4778.36 | 48.19 | 1.12 | 1.20 | NM_005777 | Hs.188879 | RBM6 |
| 3391 | 205218_at | 4779.06 | 48.21 | 1.16 | 1.27 | NM_006466 | Hs.242776 | POLR3F |
| 3392 | 208232_x_at | 4780.02 | 48.23 | 1.14 | 1.24 | L12260 | — | — |
| 3393 | 203524_s_at | 4783.80 | 48.36 | 1.13 | 1.22 | NM_021126 | Hs.248267 | MPST |
| 3394 | 218100_s_at | 4783.81 | 48.34 | 1.14 | 1.23 | NM_018010 | Hs.170318 | ESRRBL1 |
| 3395 | 208933_s_at | 4783.87 | 48.33 | 1.16 | 1.27 | AI659005 | Hs.4082 | LGALS8 |
| 3396 | 213822_s_at | 4784.84 | 48.36 | 1.11 | 1.19 | BE856776 | Hs.305834 | UBE3B |
| 3397 | 212297_at | 4785.73 | 48.37 | 1.14 | 1.24 | BF218804 | Hs.371148 | AFURS1 |
| 3398 | 200899_s_at | 4786.89 | 48.41 | 1.14 | 1.24 | NM_012215 | Hs.5734 | MGEA5 |
| 3399 | 202788_at | 4787.20 | 48.41 | 1.11 | 1.19 | NM_004635 | Hs.234521 | MAPKAPK3 |
| 3400 | 205453_at | 4787.48 | 48.41 | 1.05 | 1.08 | NM_002145 | Hs.290432 | HOXB2 |
| 3401 | 203511_s_at | 4787.66 | 48.41 | 1.13 | 1.22 | AF041432 | Hs.288013 | TRAPPC3 |
| 3402 | 201050_at | 4787.94 | 48.40 | 1.13 | 1.22 | NM_012268 | Hs.257008 | PLD3 |
| 3403 | 206613_s_at | 4788.26 | 48.40 | 1.21 | 1.21 | NM_005681 | Hs.153088 | TAF1A |
| 3404 | 218189_s_at | 4788.81 | 48.41 | 1.11 | 1.19 | NM_018946 | Hs.274424 | NANS |
| 3405 | 213402_at | 4789.32 | 48.42 | 1.13 | 1.23 | AA029811 | Hs.397153 | LOC126208 |
| 3406 | 207795_s_at | 4790.24 | 48.44 | 1.00 | 1.00 | AB009597 | Hs.41682 | KLRD1 |
| 3407 | 200685_at | 4791.28 | 48.47 | 1.13 | 1.21 | AU146237 | Hs.443458 | SFRS11 |
| 3408 | 203015_s_at | 4792.27 | 48.50 | 1.12 | 1.20 | AW136988 | Hs.22587 | SSX2IP |
| 3409 | 205728_at | 4792.38 | 48.49 | 1.06 | 1.11 | AL022718 | Hs.349094 | SH2D1A |
| 3410 | 212015_x_at | 4793.26 | 48.51 | 1.15 | 1.26 | BF690062 | Hs.172550 | PTBP1 |
| 3411 | 211792_s_at | 4793.68 | 48.51 | 1.13 | 1.22 | U17074 | Hs.4854 | CDKN2C |
| 3412 | 212173_at | 4794.81 | 48.55 | 1.14 | 1.24 | AU154985 | Hs.294008 | AK2 |
| 3413 | 209403_at | 4796.30 | 48.59 | 1.11 | 1.18 | AL136860 | Hs.462906 | TBC1D3 |
| 3414 | 208765_s_at | 4796.45 | 48.59 | 1.12 | 1.21 | NM_005826 | Hs.15265 | HNRPR |
| 3415 | 209081_s_at | 4796.92 | 48.59 | 1.06 | 1.11 | NM_030582 | Hs.413175 | COL18A1 |
| 3416 | 217140_s_at | 4796.94 | 48.57 | 1.11 | 1.18 | AJ002428 | Hs.404814 | VDAC1 |
| 3417 | 209788_s_at | 4797.84 | 48.60 | 1.00 | 1.00 | AF183569 | Hs.436186 | ARTS-1 |
| 3418 | 212318_at | 4798.60 | 48.62 | 1.11 | 1.18 | NM_012470 | Hs.412527 | TNPO3 |
| 3419 | 209463_s_at | 4799.62 | 48.65 | 1.15 | 1.26 | D50544 | Hs.421646 | TAF12 |
| 3420 | 201079_at | 4801.52 | 48.70 | 1.14 | 1.25 | NM_004710 | Hs.433753 | SYNGR2 |
| 3421 | 212182_at | 4803.32 | 48.77 | 1.13 | 1.22 | AB007956 | Hs.355399 | NUDT4 |
| 3422 | 212388_at | 4803.39 | 48.75 | 1.15 | 1.25 | AB028980 | Hs.7243 | USP24 |
| 3423 | 221789_x_at | 4804.07 | 48.76 | 1.13 | 1.23 | AK024450 | Hs.444178 | RHOT2 |
| 3424 | 217986_s_at | 4804.28 | 48.76 | 1.14 | 1.24 | NM_013448 | Hs.436468 | BAZ1A |
| 3425 | 214269_at | 4805.61 | 48.80 | 1.10 | 1.17 | AK025922 | Hs.334789 | FLJ22269 |
| 3426 | 211837_s_at | 4806.01 | 48.80 | 1.11 | 1.19 | AF165312 | — | — |
| 3427 | 219228_at | 4807.49 | 48.86 | 1.14 | 1.24 | NM_018555 | Hs.147644 | ZNF331 |
| 3428 | 201613_s_at | 4807.65 | 48.85 | 1.14 | 1.24 | BC000519 | Hs.343244 | AP1G2 |
| 3429 | 205165_at | 4808.87 | 48.89 | 1.12 | 1.21 | NM_001407 | Hs.55173 | CELSR3 |
| 3430 | 214048_at | 4810.29 | 48.93 | 1.14 | 1.25 | AI913365 | Hs.35947 | MBD4 |
| 3431 | 202356_s_at | 4810.87 | 48.94 | 1.12 | 1.21 | NM_002096 | — | — |
| 3432 | 202384_s_at | 4811.74 | 48.96 | 1.14 | 1.25 | AW167713 | Hs.301266 | TCOF1 |
| 3433 | 201608_s_at | 4812.25 | 48.97 | 1.14 | 1.24 | NM_007062 | Hs.172589 | PWP1 |
| 3434 | 212120_at | 4812.31 | 48.96 | 1.13 | 1.22 | BE897886 | Hs.442989 | RHOQ |
| 3435 | 220239_at | 4812.37 | 48.95 | 1.11 | 1.18 | NM_018846 | Hs.376793 | KLHL7 |
| 3436 | 214527_s_at | 4812.70 | 48.95 | 1.12 | 1.21 | AB041836 | Hs.30570 | PQBP1 |
| 3437 | 214597_at | 4813.34 | 48.95 | 1.13 | 1.23 | BC000256 | Hs.184841 | SSTR2 |
| 3438 | 212380_at | 4814.30 | 48.97 | 1.09 | 1.16 | D43949 | Hs.202331 | KIAA0082 |
| 3439 | 219297_at | 4814.78 | 48.98 | 1.14 | 1.25 | NM_019045 | Hs.98510 | WDR44 |
| 3440 | 221503_s_at | 4815.14 | 48.98 | 1.09 | 1.15 | AF034756 | Hs.407765 | KPNA3 |
| 3441 | 205996_s_at | 4816.47 | 49.01 | 1.14 | 1.24 | NM_013411 | Hs.294008 | AK2 |
| 3442 | 201470_at | 4818.48 | 49.08 | 1.10 | 1.17 | NM_004832 | Hs.11465 | GSTO1 |
| 3443 | 211963_s_at | 4818.70 | 49.08 | 1.12 | 1.21 | AL516350 | Hs.126222 | ARPC5 |
| 3444 | 208111_at | 4818.86 | 49.07 | 1.14 | 1.24 | NM_000054 | Hs.2524 | AVPR2 |
| 3445 | 209178_at | 4818.86 | 49.06 | 1.15 | 1.25 | AF038391 | Hs.512858 | DHX38 |
| 3446 | 201457_x_at | 4819.25 | 49.06 | 1.09 | 1.15 | AF081496 | Hs.418533 | BUB3 |
| 3447 | 212229_s_at | 4819.76 | 49.07 | 1.10 | 1.17 | AK001699 | Hs.184227 | FBXO21 |
| 3448 | 221903_s_at | 4822.89 | 49.17 | 1.14 | 1.23 | BE046443 | Hs.386952 | CYLD |
| 3449 | 211563_s_at | 4823.27 | 49.17 | 1.14 | 1.23 | AB006572 | Hs.7943 | C19orf2 |
| 3450 | 212148_at | 4824.11 | 49.20 | 1.08 | 1.14 | AL049381 | Hs.408222 | PBX1 |
| 3451 | 221571_at | 4825.98 | 49.26 | 1.14 | 1.24 | AI721219 | Hs.297660 | TRAF3 |
| 3452 | 212621_at | 4826.45 | 49.26 | 1.13 | 1.22 | AB006624 | Hs.14912 | KIAA0286 |
| 3453 | 203193_at | 4826.48 | 49.25 | 1.13 | 1.22 | NM_004451 | — | — |
| 3454 | 200701_at | 4827.41 | 49.27 | 1.06 | 1.11 | NM_006432 | Hs.433222 | NPC2 |
| 3455 | 215083_at | 4827.59 | 49.26 | 1.08 | 1.14 | AL049263 | Hs.507407 | — |
| 3456 | 215236_s_at | 4828.67 | 49.29 | 1.11 | 1.19 | AV721177 | Hs.39252 | PICALM |
| 3457 | 203822_s_at | 4829.57 | 49.31 | 1.14 | 1.24 | NM_006874 | Hs.82143 | ELF2 |
| 3458 | 202880_s_at | 4830.10 | 49.32 | 1.13 | 1.22 | NM_004762 | Hs.1050 | PSCD1 |
| 3459 | 222074_at | 4830.44 | 49.32 | 1.14 | 1.23 | AW614435 | Hs.78601 | UROD |
| 3460 | 202727_s_at | 4830.95 | 49.33 | 1.13 | 1.22 | NM_000416 | Hs.180866 | IFNGR1 |
| 3461 | 212423_at | 4831.12 | 49.32 | 1.14 | 1.23 | AK024784 | Hs.28264 | C10orf56 |
| 3462 | 202798_at | 4832.17 | 49.35 | 1.16 | 1.28 | NM_006323 | Hs.7239 | SEC24B |
| 3463 | 209574_s_at | 4833.15 | 49.37 | 1.15 | 1.26 | AI349506 | Hs.285091 | C18orf1 |
| 3464 | 201360_at | 4833.24 | 49.36 | 1.12 | 1.20 | NM_000099 | Hs.304682 | CST3 |
| 3465 | 210166_at | 4833.42 | 49.36 | 1.03 | 1.06 | AF051151 | Hs.114408 | TLR5 |
| 3466 | 207118_s_at | 4834.24 | 49.38 | 1.13 | 1.22 | NM_004659 | Hs.211819 | MMP23A /// |
| | | | | | | | | MMP23B |
| 3467 | 222128_at | 4835.29 | 49.41 | 1.09 | 1.14 | U80764 | Hs.435786 | CACNB2 |
| 3468 | 218638_s_at | 4835.49 | 49.40 | 0.96 | 0.94 | NM_012445 | Hs.435953 | SPON2 |
| 3469 | 212193_s_at | 4837.34 | 49.48 | 1.12 | 1.21 | BE881529 | Hs.6214 | LARP |
| 3470 | 211975_at | 4838.19 | 49.50 | 1.13 | 1.23 | BE299671 | Hs.436204 | ZNF289 |
| 3471 | 201514_s_at | 4838.22 | 49.49 | 1.15 | 1.25 | NM_005754 | Hs.3353 | G3BP |
| 3472 | 212074_at | 4838.35 | 49.48 | 1.03 | 1.04 | BE972774 | Hs.438072 | UNC84A |
| 3473 | 212296_at | 4838.72 | 49.48 | 1.11 | 1.19 | NM_005805 | Hs.178761 | PSMD14 |
| 3474 | 201791_s_at | 4839.68 | 49.51 | 1.14 | 1.24 | NM_001360 | Hs.11806 | DHCR7 |
| 3475 | 44790_s_at | 4840.03 | 49.51 | 1.13 | 1.23 | AI129310 | Hs.413071 | C13orf18 |
| 3476 | 210943_s_at | 4840.69 | 49.52 | 1.01 | 1.01 | U84744 | Hs.130188 | CHS1 |
| 3477 | 217503_at | 4841.77 | 49.55 | 1.12 | 1.20 | AA203487 | — | — |
| 3478 | 221005_s_at | 4842.45 | 49.55 | 1.13 | 1.22 | NM_030783 | Hs.12851 | PTDSS2 |
| 3479 | 209724_s_at | 4842.53 | 49.54 | 1.14 | 1.24 | AL534416 | Hs.156000 | ZFP161 |
| 3480 | 210422_x_at | 4843.42 | 49.57 | 1.09 | 1.15 | D50402 | Hs.135163 | SLC11A1 |
| 3481 | 203239_s_at | 4843.45 | 49.55 | 1.13 | 1.21 | NM_014516 | Hs.343571 | CNOT3 |
| 3482 | 203298_s_at | 4843.84 | 49.56 | 1.14 | 1.24 | NM_004973 | Hs.40154 | JARID2 |
| 3483 | 209267_s_at | 4845.45 | 49.61 | 1.02 | 1.04 | AB040120 | Hs.284205 | SLC39A8 |
| 3484 | 202681_at | 4846.90 | 49.66 | 1.14 | 1.23 | AI346043 | Hs.77500 | USP4 |
| 3485 | 220558_x_at | 4847.28 | 49.66 | 1.13 | 1.22 | NM_005705 | Hs.271954 | PHEMX |
| 3486 | 221754_s_at | 4847.53 | 49.65 | 1.13 | 1.21 | AI341234 | Hs.6191 | CORO1B |
| 3487 | 212321_at | 4847.64 | 49.65 | 1.13 | 1.22 | AB033078 | Hs.186613 | SGPL1 |
| 3488 | 216095_x_at | 4849.44 | 49.70 | 1.15 | 1.26 | AF057354 | Hs.347187 | MTMR1 |
| 3489 | 204859_s_at | 4849.78 | 49.70 | 1.07 | 1.12 | NM_013229 | Hs.373575 | APAF1 |
| 3490 | 201076_at | 4850.20 | 49.71 | 1.15 | 1.25 | NM_005008 | Hs.182255 | NHP2L1 |
| 3491 | 200958_s_at | 4850.22 | 49.69 | 1.14 | 1.24 | NM_005625 | Hs.164067 | SDCBP |
| 3492 | 209310_s_at | 4850.42 | 49.69 | 1.09 | 1.15 | U25804 | Hs.74122 | CASP4 |
| 3493 | 219077_s_at | 4850.78 | 49.69 | 1.06 | 1.10 | NM_016373 | Hs.519 | WWOX |
| 3494 | 210162_s_at | 4850.85 | 49.68 | 1.14 | 1.24 | U08015 | Hs.96149 | NFATC1 |
| 3495 | 212590_at | 4852.17 | 49.71 | 1.11 | 1.20 | AI431643 | Hs.206097 | RRAS2 |
| 3496 | 202313_at | 4852.33 | 49.71 | 1.14 | 1.24 | NM_002717 | Hs.512628 | PPP2R2A |
| 3497 | 201806_s_at | 4852.45 | 49.70 | 1.12 | 1.20 | NM_007245 | Hs.43509 | ATXN2L |
| 3498 | 203005_at | 4852.55 | 49.69 | 1.10 | 1.17 | NM_002342 | Hs.1116 | LTBR |
| 3499 | 218945_at | 4853.48 | 49.72 | 1.09 | 1.16 | NM_024109 | Hs.306380 | MGC2654 |
| 3500 | 210743_s_at | 4854.39 | 49.74 | 1.15 | 1.26 | AF064103 | Hs.405214 | CDC14A |
| 3501 | 212658_at | 4855.05 | 49.75 | 1.08 | 1.14 | N66633 | Hs.79299 | LHFPL2 |
| 3502 | 201918_at | 4855.18 | 49.74 | 0.98 | 0.97 | AI927944 | Hs.144130 | FLJ10618 |
| 3503 | 203604_at | 4855.77 | 49.75 | 1.12 | 1.21 | N38750 | Hs.458486 | ZNF516 |
| 3504 | 213830_at | 4855.93 | 49.74 | 0.93 | 0.89 | AW007751 | Hs.2014 | TRD@ |
| 3505 | 220704_at | 4857.19 | 49.78 | 1.04 | 1.06 | NM_018563 | — | — |
| 3506 | 201720_s_at | 4857.78 | 49.79 | 1.13 | 1.22 | AI589086 | Hs.436200 | LAPTM5 |
| 3507 | 217620_s_at | 4857.84 | 49.78 | 1.10 | 1.18 | AA805318 | Hs.239818 | PIK3CB |
| 3508 | 201993_x_at | 4859.39 | 49.83 | 1.15 | 1.25 | NM_005463 | Hs.372673 | HNRPDL |
| 3509 | 218228_s_at | 4859.40 | 49.81 | 1.11 | 1.18 | NM_025235 | Hs.280776 | TNKS2 |
| 3510 | 200775_s_at | 4860.01 | 49.82 | 1.14 | 1.23 | BC000355 | Hs.307544 | HNRPK |
| 3511 | 203195_s_at | 4860.08 | 49.81 | 1.15 | 1.26 | NM_005387 | Hs.112255 | NUP98 |
| 3512 | 203893_at | 4861.81 | 49.87 | 1.10 | 1.17 | NM_016283 | Hs.60679 | TAF9 |
| 3513 | 34225_at | 4863.64 | 49.93 | 1.14 | 1.25 | AF101434 | Hs.21771 | WHSC2 |
| 3514 | 202665_s_at | 4863.81 | 49.92 | 1.14 | 1.23 | NM_003387 | Hs.401414 | WASPIP |
| 3515 | 201168_x_at | 4865.61 | 49.97 | 1.12 | 1.20 | NM_004309 | Hs.159161 | ARHGDIA |
| 3516 | 217850_at | 4865.78 | 49.97 | 1.15 | 1.25 | NM_014366 | Hs.313544 | NS |
| 3517 | 218921_at | 4865.97 | 49.96 | 1.11 | 1.19 | NM_021805 | Hs.433036 | SIGIRR |
| 3518 | 211376_s_at | 4866.57 | 49.98 | 1.16 | 1.27 | BC005212 | Hs.258798 | C10orf86 |
| 3519 | 202458_at | 4867.27 | 49.99 | 1.03 | 1.05 | NM_007173 | Hs.25338 | PRSS23 |
| 3520 | 211252_x_at | 4867.48 | 49.98 | 1.05 | 1.08 | U36759 | — | — |

| 0 | Title |
|---|---|
| 1 | hemoglobin, gamma A |
| 2 | defensin, alpha 1, myeloid-related sequence |
| 3 | lactotransferrin |
| 4 | integrin, alpha 2b (platelet glycoprotein IIb of IIb/IIIa complex, antigen CD41B) |
| 5 | Charcot-Leyden crystal protein |
| 6 | chemokine (C—X—C motif) receptor 4 |
| 7 | hemoglobin, gamma A |
| 8 | integrin, beta 3 (platelet glycoprotein IIIa, antigen CD61) |
| 9 | chemokine (C—X—C motif) receptor 4 /// chemokine (C—X—C motif) receptor 4 |
| 10 | integrin, alpha 2b (platelet glycoprotein IIb of IIb/IIIa complex, antigen CD41B) |
| 11 | hemoglobin, delta |
| 12 | hemoglobin, gamma A |
| 13 | pre-B-cell colony enhancing factor 1 |
| 14 | integrin, alpha 2b (platelet glycoprotein IIb of IIb/IIIa complex, antigen CD41B) |
| 15 | serine/threonine kinase 17b (apoptosis-inducing) |
| 16 | — |
| 17 | annexin A3 |
| 18 | vanin 1 |
| 19 | myosin, light polypeptide 9, regulatory |
| 20 | — |
| 21 | chondroitin sulfate proteoglycan 2 (versican) |
| 22 | histone 2, H2be |
| 23 | defensin, alpha 4, corticostatin |
| 24 | aminolevulinate, delta-, synthase 2 (sideroblastic/hypochromic anemia) |
| 25 | tropomyosin 1 (alpha) |
| 26 | DEAD (Asp-Glu-Ala-Asp) box polypeptide 3, X-linked |
| 27 | splicing factor proline/glutamine rich (polypyrimidine tract binding protein associated) |
| 28 | |
| 29 | carcinoembryonic antigen-related cell adhesion molecule 8 |
| 30 | hypothetical protein MGC14376 |
| 31 | histone 1, H3h |
| 32 | serine palmitoyltransferase, long chain base subunit 2 |
| 33 | progesterone receptor membrane component 1 |
| 34 | nuclear receptor subfamily 4, group A, member 2 |
| 35 | CD36 antigen (collagen type I receptor, thrombospondin receptor) |
| 36 | lipocalin 2 (oncogene 24p3) |
| 37 | glycoprotein Ib (platelet), beta polypeptide |
| 38 | transcription factor 7-like 2 (T-cell specific, HMG-box) |
| 39 | nuclear receptor subfamily 4, group A, member 2 |
| 40 | hemoglobin, beta |
| 41 | membrane-spanning 4-domains, subfamily A, member 3 (hematopoietic cell-specific) |
| 42 | solute carrier family 24 (sodium/potassium/calcium exchanger), member 3 |
| 43 | clusterin (complement lysis inhibitor, SP-40,40, sulfated glycoprotein 2, testosterone-repressed prostate message 2, |
| | apolipoprotein J) |
| 44 | major histocompatibility complex, class II, DR beta 4 |
| 45 | proprotein convertase subtilisin/kexin type 6 |
| 46 | CTD (carboxy-terminal domain, RNA polymerase II, polypeptide A) small phosphatase-like |
| 47 | synuclein, alpha (non A4 component of amyloid precursor) |
| 48 | hemoglobin, alpha 2 |
| 49 | hypothetical protein MGC27165 |
| 50 | Ras suppressor protein 1 |
| 51 | ataxin 1 |
| 52 | hemoglobin, alpha 2 /// hemoglobin, alpha 2 |
| 53 | CD9 antigen (p24) |
| 54 | CTD (carboxy-terminal domain, RNA polymerase II, polypeptide A) small phosphatase-like |
| 55 | cathelicidin antimicrobial peptide |
| 56 | prostaglandin-endoperoxide synthase 1 (prostaglandin G/H synthase and cyclooxygenase) |
| 57 | major histocompatibility complex, class II, DQ beta 1 |
| 58 | — |
| 59 | villin 2 (ezrin) |
| 60 | thyroid hormone receptor associated protein 2 |
| 61 | serum/glucocorticoid regulated kinase |
| 62 | CDNA FLJ43139 fis, clone CTONG3007444 |
| 63 | clusterin (complement lysis inhibitor, SP-40,40, sulfated glycoprotein 2, testosterone-repressed prostate message 2, |
| | apolipoprotein J) |
| 64 | WD40 repeat protein interacting with phosphoinositides of 49 kDa |
| 65 | hypothetical protein DKFZp547A023 |
| 66 | eukaryotic translation initiation factor 4A, isoform 1 |
| 67 | putative translation initiation factor |
| 68 | nicotinamide nucleotide transhydrogenase |
| 69 | pre-B-cell colony enhancing factor 1 |
| 70 | cysteine-rich secretory protein 3 |
| 71 | secreted protein, acidic, cysteine-rich (osteonectin) |
| 72 | amyloid beta (A4) precursor-like protein 2 |
| 73 | hemoglobin, alpha 2 |
| 74 | chitinase 3-like 1 (cartilage glycoprotein-39) |
| 75 | early growth response 1 |
| 76 | MRNA full length insert cDNA clone EUROIMAGE 27080 |
| 77 | tubulin, beta 1 /// tubulin, beta 1 |
| 78 | hematopoietically expressed homeobox |
| 79 | thrombospondin 1 |
| 80 | nuclear receptor subfamily 4, group A, member 2 |
| 81 | serum deprivation response (phosphatidylserine binding protein) |
| 82 | syntaxin 11 |
| 83 | HBS1-like (S. cerevisiae) |
| 84 | Rho GTPase activating protein 6 |
| 85 | immunoglobulin J polypeptide, linker protein for immunoglobulin alpha and mu polypeptides |
| 86 | interferon gamma receptor 1 /// interferon gamma receptor 1 |
| 87 | chondroitin sulfate proteoglycan 2 (versican) |
| 88 | selenoprotein T |
| 89 | RNA binding motif, single stranded interacting protein 1 |
| 90 | TRAF2 and NCK interacting kinase |
| 91 | SNF1-like kinase /// SNF1-like kinase |
| 92 | S100 calcium binding protein P |
| 93 | histone 1, H2bg |
| 94 | — |
| 95 | integrin, beta 3 (platelet glycoprotein IIIa, antigen CD61) |
| 96 | caspase 1, apoptosis-related cysteine protease (interleukin 1, beta, convertase) |
| 97 | phospholipase C-like 2 |
| 98 | ataxin 7 |
| 99 | major histocompatibility complex, class II, DQ alpha 1 |
| 100 | ring finger protein 10 |
| 101 | — |
| 102 | GDP-mannose 4,6-dehydratase |
| 103 | transcription factor EC |
| 104 | Kell blood group precursor (McLeod phenotype) |
| 105 | thioredoxin domain containing /// thioredoxin domain containing |
| 106 | heterogeneous nuclear ribonucleoprotein D (AU-rich element RNA binding protein 1, 37 kDa) |
| 107 | bactericidal/permeability-increasing protein |
| 108 | tropomyosin 1 (alpha) |
| 109 | dynactin 1 (p150, glued homolog, Drosophila) |
| 110 | histone 1, H2ae |
| 111 | carbonic anhydrase II |
| 112 | alpha thalassemia/mental retardation syndrome X-linked (RAD54 homolog, S. cerevisiae) |
| 113 | major histocompatibility complex, class II, DQ alpha 2 /// major histocompatibility complex, class II, DQ alpha 1 |
| 114 | C-type lectin-like receptor-2 |
| 115 | protein kinase, cAMP-dependent, regulatory, type II, beta |
| 116 | dual specificity phosphatase 2 |
| 117 | protein kinase, lysine deficient 1 |
| 118 | ataxia telangiectasia mutated (includes complementation groups A, C and D) |
| 119 | component of oligomeric golgi complex 5 |
| 120 | transcription factor 7-like 2 (T-cell specific, HMG-box) |
| 121 | tyrosine 3-monooxygenase/tryptophan 5-monooxygenase activation protein, eta polypeptide |
| 122 | synuclein, alpha (non A4 component of amyloid precursor) |
| 123 | heterogeneous nuclear ribonucleoprotein A1 |
| 124 | a disintegrin and metalloproteinase domain 28 |
| 125 | ring finger and CHY zinc finger domain containing 1 |
| 126 | superoxide dismutase 2, mitochondrial |
| 127 | neurogranin (protein kinase C substrate, RC3) |
| 128 | heterogeneous nuclear ribonucleoprotein L |
| 129 | TGFB inducible early growth response 2 |
| 130 | v-ets erythroblastosis virus E26 oncogene homolog 2 (avian) |
| 131 | neuregulin 1 |
| 132 | aldehyde dehydrogenase 1 family, member A1 |
| 133 | immunoglobulin heavy constant mu |
| 134 | T-cell acute lymphocytic leukemia 1 |
| 135 | hemoglobin, alpha 2 /// hemoglobin, alpha 2 |
| 136 | immunoglobulin lambda constant 2 (Kern-Oz-marker) |
| 137 | — |
| 138 | ribosomal protein L10 |
| 139 | protein kinase C, delta |
| 140 | NADPH cytochrome B5 oxidoreductase |
| 141 | — |
| 142 | cylindromatosis (turban tumor syndrome) |
| 143 | E3 identified by differential display |
| 144 | guanine nucleotide binding protein (G protein), alpha z polypeptide |
| 145 | Ras-induced senescence 1 |
| 146 | guanosine monophosphate reductase |
| 147 | development and differentiation enhancing factor 1 |
| 148 | RAB31, member RAS oncogene family |
| 149 | growth arrest-specific 2 like 1 |
| 150 | tumor necrosis factor, alpha-induced protein 3 |
| 151 | interleukin enhancer binding factor 3, 90 kDa |
| 152 | hemoglobin, alpha 2 |
| 153 | guanine nucleotide binding protein (G protein), alpha inhibiting activity polypeptide 3 |
| 154 | eukaryotic translation initiation factor 5A |
| 155 | tryptophanyl-tRNA synthetase |
| 156 | mannosyl (alpha-1,3-)-glycoprotein beta-1,4-N-acetylglucosaminyltransferase, isoenzyme B |
| 157 | caldesmon 1 |
| 158 | |
| 159 | acyl-CoA synthetase long-chain family member 1 |
| 160 | CDC14 cell division cycle 14 homolog B (S. cerevisiae) |
| 161 | protein tyrosine phosphatase, non-receptor type 22 (lymphoid) |
| 162 | integrin, alpha 4 (antigen CD49D, alpha 4 subunit of VLA-4 receptor) |
| 163 | junctional adhesion molecule 3 |
| 164 | transcription factor 7-like 2 (T-cell specific, HMG-box) |
| 165 | immunoglobulin heavy constant gamma 1 (G1m marker) |
| 166 | WW domain binding protein 11 |
| 167 | hypothetical protein FLJ21616 |
| 168 | calmodulin 1 (phosphorylase kinase, delta) |
| 169 | chemokine (C—X—C motif) ligand 5 |
| 170 | monoglyceride lipase /// monoglyceride lipase |
| 171 | suppression of tumorigenicity 14 (colon carcinoma, matriptase, epithin) |
| 172 | ubiquitin-conjugating enzyme E2, J1 (UBC6 homolog, yeast) |
| 173 | IGF-IImRNA-binding protein 3 |
| 174 | H3 histone, family 3B (H3.3B) |
| 175 | WD repeat domain 41 |
| 176 | FBJ murine osteosarcoma viral oncogene homolog B |
| 177 | CD83 antigen (activated B lymphocytes, immunoglobulin superfamily) |
| 178 | putative lymphocyte G0/G1 switch gene |
| 179 | dynamin 3 |
| 180 | LR8 protein |
| 181 | SWI/SNF related, matrix associated, actin dependent regulator of chromatin, subfamily c, member 1 |
| 182 | histone 1, H2ac |
| 183 | prostaglandin-endoperoxide synthase 2 (prostaglandin G/H synthase and cyclooxygenase) |
| 184 | follistatin-like 1 |
| 185 | FK506 binding protein 1B, 12.6 kDa |
| 186 | — |
| 187 | H2.0-like homeo box 1 (Drosophila) |
| 188 | stromal antigen 2 |
| 189 | RAD23 homolog B (S. cerevisiae) |
| 190 | v-rel reticuloendotheliosis viral oncogene homolog (avian) |
| 191 | junction plakoglobin |
| 192 | protein tyrosine phosphatase, non-receptor type 12 |
| 193 | hypothetical protein FLJ23556 |
| 194 | TAF15 RNA polymerase II, TATA box binding protein (TBP)-associated factor, 68 kDa |
| 195 | serine/arginine repetitive matrix 1 |
| 196 | SET binding protein 1 |
| 197 | autocrine motility factor receptor |
| 198 | tribbles homolog 1 (Drosophila) |
| 199 | 3-hydroxy-3-methylglutaryl-Coenzyme A reductase |
| 200 | amyloid beta (A4) precursor-like protein 2 |
| 201 | calpain 3, (p94) |
| 202 | major histocompatibility complex, class II, DP alpha 1 |
| 203 | Fc fragment of IgG, low affinity IIIa, receptor for (CD16) |
| 204 | prostaglandin-endoperoxide synthase 1 (prostaglandin G/H synthase and cyclooxygenase) |
| 205 | tropomyosin 1 (alpha) |
| 206 | guanylate cyclase 1, soluble, alpha 3 |
| 207 | reticulon 1 |
| 208 | immunoglobulin lambda constant 2 (Kern-Oz-marker) |
| 209 | cytochrome P450, family 4, subfamily F, polypeptide 3 |
| 210 | discs, large homolog 5 (Drosophila) |
| 211 | inositol(myo)-1(or 4)-monophosphatase 2 |
| 212 | toll-like receptor 8 |
| 213 | nidogen (enactin) |
| 214 | — |
| 215 | SH3-domain binding protein 3 |
| 216 | platelet/endothelial cell adhesion molecule (CD31 antigen) |
| 217 | N-acetylgalactosaminidase, alpha- |
| 218 | mediator of RNA polymerase II transcription, subunit 6 homolog (yeast) |
| 219 | hemoglobin, beta /// hemoglobin, beta |
| 220 | hexokinase 2 |
| 221 | hypothetical protein LOC150759 |
| 222 | interleukin 18 receptor 1 |
| 223 | RAN binding protein 9 |
| 224 | period homolog 1 (Drosophila) |
| 225 | — |
| 226 | RNA binding motif, single stranded interacting protein 1 |
| 227 | src family associated phosphoprotein 2 |
| 228 | egf-like module containing, mucin-like, hormone receptor-like 2 |
| 229 | NACHT, leucine rich repeat and PYD containing 1 |
| 230 | phosphogluconate dehydrogenase |
| 231 | KIAA0830 protein |
| 232 | — |
| 233 | CD69 antigen (p60, early T-cell activation antigen) |
| 234 | SH2 domain containing phosphatase anchor protein 1 /// SH2 domain containing phosphatase anchor protein 1 |
| 235 | serine palmitoyltransferase, long chain base subunit 2 |
| 236 | tubulin, beta polypeptide |
| 237 | transmembrane 6 superfamily member 1 |
| 238 | Ras-related GTP binding D |
| 239 | ataxia telangiectasia mutated (includes complementation groups A, C and D) |
| 240 | Meis1, myeloid ecotropic viral integration site 1 homolog (mouse) |
| 241 | transferrin receptor (p90, CD71) |
| 242 | SH3-domain GRB2-like endophilin B1 |
| 243 | protein kinase, cAMP-dependent, regulatory, type I, alpha (tissue specific extinguisher 1) |
| 244 | integrin, beta 5 |
| 245 | N-acylsphingosine amidohydrolase (acid ceramidase)-like |
| 246 | RNA binding motif, single stranded interacting protein 1 |
| 247 | CDNA FLJ12108 fis, clone MAMMA1000009 |
| 248 | family with sequence similarity 46, member C |
| 249 | microtubule-actin crosslinking factor 1 |
| 250 | transgelin |
| 251 | 4-aminobutyrate aminotransferase |
| 252 | zinc finger protein 185 (LIM domain) |
| 253 | 3′-phosphoadenosine 5′-phosphosulfate synthase 1 |
| 254 | acid phosphatase 1, soluble |
| 255 | microfibrillar-associated protein 3-like |
| 256 | protein kinase, DNA-activated, catalytic polypeptide |
| 257 | transducin (beta)-like 1X-linked receptor 1 /// transducin (beta)-like 1X-linked receptor 1 |
| 258 | chromosome 6 open reading frame 79 |
| 259 | glutamate-ammonia ligase (glutamine synthase) |
| 260 | growth factor receptor-bound protein 14 |
| 261 | chromosome 3 open reading frame 4 |
| 262 | glucose-6-phosphate dehydrogenase |
| 263 | src family associated phosphoprotein 2 |
| 264 | collagen, type VI, alpha 2 |
| 265 | clusterin (complement lysis inhibitor, SP-40,40, sulfated glycoprotein 2, testosterone-repressed prostate message 2, |
| | apolipoprotein J) |
| 266 | major histocompatibility complex, class II, DR alpha |
| 267 | pellino homolog 1 (Drosophila) |
| 268 | tumor necrosis factor receptor superfamily, member 17 |
| 269 | KIAA0998 |
| 270 | tousled-like kinase 1 |
| 271 | arginase, type II |
| 272 | RAB6 interacting protein 1 |
| 273 | CDC14 cell division cycle 14 homolog B (S. cerevisiae) /// CDC14 cell division cycle 14 homolog B (S. cerevisiae) |
| 274 | DEAH (Asp-Glu-Ala-His) box polypeptide 15 |
| 275 | pinin, desmosome associated protein |
| 276 | GDP dissociation inhibitor 2 /// GDP dissociation inhibitor 2 |
| 277 | KIAA0540 protein |
| 278 | CD44 antigen (homing function and Indian blood group system) |
| 279 | hypothetical protein TI-227H |
| 280 | olfactomedin 4 |
| 281 | ADP-ribosylation factor 1 |
| 282 | arginase, liver |
| 283 | protein tyrosine phosphatase type IVA, member 1 |
| 284 | myosin, light polypeptide kinase |
| 285 | polymerase (DNA directed) sigma |
| 286 | Rap guanine nucleotide exchange factor (GEF) 2 |
| 287 | stannin |
| 288 | glutamate dehydrogenase 1 |
| 289 | mitogen-activated protein kinase kinase kinase kinase 4 |
| 290 | glutamate-ammonia ligase (glutamine synthase) |
| 291 | hypothetical protein LOC51257 |
| 292 | thrombospondin 1 |
| 293 | immunoglobulin lambda constant 2 (Kern-Oz-marker) |
| 294 | peroxisomal Ion protease |
| 295 | calpain 7 |
| 296 | aldehyde dehydrogenase 8 family, member A1 |
| 297 | protein kinase, lysine deficient 1 |
| 298 | casein kinase 1, delta |
| 299 | 2′,5′-oligoadenylate synthetase 1, 40/46 kDa |
| 300 | NACHT, leucine rich repeat and PYD containing 1 |
| 301 | adrenergic, beta, receptor kinase 1 |
| 302 | ubiquitously transcribed tetratricopeptide repeat, X chromosome |
| 303 | nuclear factor (erythroid-derived 2), 45 kDa |
| 304 | arachidonate 5-lipoxygenase |
| 305 | — |
| 306 | actin binding LIM protein family, member 3 |
| 307 | enolase 2 (gamma, neuronal) |
| 308 | actinin, alpha 1 |
| 309 | hypothetical protein LOC150759 |
| 310 | chondroitin sulfate proteoglycan 6 (bamacan) |
| 311 | F11 receptor |
| 312 | dynamin 1-like |
| 313 | KIAA0692 protein |
| 314 | early growth response 2 (Krox-20 homolog, Drosophila) |
| 315 | REST corepressor 3 |
| 316 | matrix metalloproteinase 9 (gelatinase B, 92 kDa gelatinase, 92 kDa type IV collagenase) |
| 317 | cyclin L2 /// cyclin L2 |
| 318 | RAB31, member RAS oncogene family |
| 319 | RALBP1 associated Eps domain containing 1 |
| 320 | major histocompatibility complex, class II, DQ beta 1 |
| 321 | src family associated phosphoprotein 2 |
| 322 | armadillo repeat containing 8 |
| 323 | membrane associated DNA binding protein |
| 324 | Similar to immunoglobulin kappa light chain VC region |
| 325 | mitogen-activated protein kinase 1 |
| 326 | ATP-binding cassette, sub-family A (ABC1), member 7 |
| 327 | BIA2 |
| 328 | histidine decarboxylase |
| 329 | selectin P (granule membrane protein 140 kDa, antigen CD62) |
| 330 | ubiquitin-conjugating enzyme E2, J1 (UBC6 homolog, yeast) |
| 331 | ATPase, H+ transporting, lysosomal 56/58 kDa, V1 subunit B, isoform 2 |
| 332 | muscleblind-like (Drosophila) |
| 333 | parvin, beta |
| 334 | RAB1A, member RAS oncogene family |
| 335 | synuclein, alpha (non A4 component of amyloid precursor) |
| 336 | tumor protein D52 |
| 337 | integrin beta 1 binding protein 1 |
| 338 | slingshot homolog 1 (Drosophila) |
| 339 | amyloid beta (A4) precursor-like protein 2 |
| 340 | CCR4-NOT transcription complex, subunit 8 |
| 341 | F-box and WD-40 domain protein 7 (archipelago homolog, Drosophila) |
| 342 | multimerin 1 |
| 343 | regulator of G-protein signalling 1 |
| 344 | ATPase, Class II, type 9A |
| 345 | disabled homolog 2, mitogen-responsive phosphoprotein (Drosophila) |
| 346 | T-cell immunomodulatory protein /// T-cell immunomodulatory protein |
| 347 | GATA binding protein 2 |
| 348 | N-sulfoglucosamine sulfohydrolase (sulfamidase) |
| 349 | proteolipid protein 2 (colonic epithelium-enriched) |
| 350 | — |
| 351 | WAS protein family, member 3 |
| 352 | CDNA FLJ35626 fis, clone SPLEN2011086 |
| 353 | uncoupling protein 2 (mitochondrial, proton carrier) |
| 354 | zinc finger protein 318 |
| 355 | hypothetical protein DKPZp586I1420 |
| 356 | interleukin 8 |
| 357 | Clone 23872 mRNA sequence |
| 358 | — |
| 359 | acyl-Coenzyme A dehydrogenase family, member 8 |
| 360 | proteoglycan 1, secretory granule |
| 361 | heat shock 70 kDa protein 6 (HSP70B′) |
| 362 | protein kinasa C, iota |
| 363 | protein kinase, X-linked /// protein kinase, Y-linked |
| 364 | thioredoxin domain containing 5 /// thioredoxin domain containing 5 |
| 365 | monocyte to macrophage differentiation-associated |
| 366 | influenza virus NS1A binding protein |
| 367 | guanylate cyclase 1, soluble, beta 3 |
| 368 | hypothetical protein FLJ10546 |
| 369 | membrane interacting protein of RGS16 |
| 370 | — |
| 371 | immunoglobulin lambda constant 2 (Kern-Oz-marker) |
| 372 | voltage-dependent anion channel 1 |
| 373 | ectodermal-neural cortex (with BTB-like domain) |
| 374 | tumor necrosis factor (ligand) superfamily, member 4 (tax-transcriptionally activated glycoprotein 1, 34 kDa) |
| 375 | DnaJ (Hsp40) homolog, subfamily B, member 6 |
| 376 | platelet-activating factor receptor |
| 377 | CD99 antigen |
| 378 | maltase-glucoamylase (alpha-glucosidase) |
| 379 | leukocyte immunoglobulin-like receptor, subfamily A (without TM domain), member 3 |
| 380 | membrane protein, palmitoylated 1, 55 kDa |
| 381 | carboxylesterase 1 (monocyte/macrophage serine esterase 1) |
| 382 | vinculin |
| 383 | IGF-II mRNA-binding protein 3 |
| 384 | leucine rich repeat (in FLII) interacting protein 2 |
| 385 | thioredoxin domain containing 3 (spermatozoa) |
| 386 | procollagen-proline, 2-oxoglutarate 4-dioxygenasa (proline 4-hydroxylase), alpha polypeptide I |
| 387 | scaffold attachment factor B2 |
| 388 | CD27-binding (Siva) protein |
| 389 | cytochrome b-245, beta polypeptide (chronic granulomatous disease) |
| 390 | guanine nucleotide binding protein (G protein), beta 5 |
| 391 | gamma-aminobutyric acid (GABA) B receptor, 1 |
| 392 | carbohydrate (chondroitin 4) sulfotransferase 11 |
| 393 | heat shock 90 kDa protein 1, beta |
| 394 | purinergic receptor P2X, ligand-gated ion channel, 1 |
| 395 | delta sleep inducing peptide, immunoreactor |
| 396 | heterogeneous nuclear ribonucleoprotein U (scaffold attachment factor A) |
| 397 | polypyrimidine tract binding protein 1 |
| 398 | a disintegrin and metalloproteinase domain 10 |
| 399 | vesicle-associated membrane protein 3 (cellubrevin) /// vesicle-associated membrane protein 3 (cellubrevin) |
| 400 | ring finger protein 146 /// ring finger protein 146 |
| 401 | NCK adaptor protein 2 |
| 402 | Kruppel-like factor 3 (basic) |
| 403 | SWI/SNF related, matrix associated, actin dependent regulator of chromatin, subfamily a, member 2 |
| 404 | — |
| 405 | ubiquitin specific protease 15 |
| 406 | limkain bela 2 |
| 407 | protective protein for beta-galactosidase (galactosialidosis) |
| 408 | putative homeodomain transcription factor 1 |
| 409 | X-prolyl aminopeptidase (aminopeptidase P) 1, soluble |
| 410 | ribosomal protein L31 |
| 411 | methyltransferase like 3 |
| 412 | methionyl aminopeptidase 2 |
| 413 | ribosomal protein L17 |
| 414 | B-cell CLL/lymphoma 3 |
| 415 | KIAA0I25 |
| 416 | v-maf musculoaponeurotic fibrosarcoma oncogene homolog F (avian) |
| 417 | mediator of RNA polymerase II transcription, subunit 6 homolog (yeast) |
| 418 | complement component 3a receptor 1 |
| 419 | RAB31, member RAS oncogene family |
| 420 | thrombospondin 1 |
| 421 | histone 2, H2aa |
| 422 | ornithine decarboxylase 1 |
| 423 | yippee-like 5 (Drosophila) |
| 424 | transcobalamin I (vitamin B12 binding protein, R binder family) |
| 425 | serine/threonine kinase 38 |
| 426 | claudin 5 (transmembrane protein deleted in velocardiofacial syndrome) |
| 427 | guanylate cyclase 1, soluble, beta 3 |
| 428 | runt-related transcription factor 3 |
| 429 | chromosome 10 open reading frame 6 |
| 430 | degenerative spermatocyte homolog, lipid desaturase (Drosophila) |
| 431 | phosphorylase kinase, beta |
| 432 | cytoskeleton-associated protein 4 |
| 433 | tumor necrosis factor, alpha-induced protein 3 |
| 434 | phosphoinositide-3-kinase, catalytic, alpha polypeptide |
| 435 | transcription factor 7-like 2 (T-cell specific, HMG-box) |
| 436 | HIR histone cell cycle regulation defective homolog A (S. cerevisiae) |
| 437 | nascent-polypeptide-associated complex alpha polypeptide |
| 438 | ATPase, Ca++ transporting, cardiac muscle, slow twitch 2 |
| 439 | CD58 antigen, (lymphocyte function-associated antigen 3) /// CD58 antigen, (lymphocyte function-associated antigen 3) |
| 440 | ELL associated factor 2 |
| 441 | RAS p21 protein activator (GTPase activating protein) 1 |
| 442 | apolipoprotein B mRNA editing enzyme, catalytic polypeptide-like 3B |
| 443 | erythrocyte membrane protein band 4.1-like 3 /// erythrocyte membrane protein band 4.1-like 3 |
| 444 | heterogeneous nuclear ribonucleoprotein A1 |
| 445 | cortactin |
| 446 | Isolate donor Z clone Z55K immunoglobulin kappa light chain variable region mRNA, partial cds /// Isolate donor Z clone Z55K |
| | immunoglobulin kappa light chain variable region mRNA, partial cds |
| 447 | histone 2, H2aa |
| 448 | growth arrest and DNA-damage-inducible, alpha |
| 449 | chromosome 20 open reading frame 30 |
| 450 | cyclin-dependent kinase inhibitor 1A (p21, Cip1) |
| 451 | granzyme K (serine protease, granzyme 3; tryptase II) |
| 452 | filamin A, alpha (actin binding protein 280) |
| 453 | putative homeodomain transcription factor 1 |
| 454 | KIAA0368 |
| 455 | — |
| 456 | synuclein, alpha (non A4 component of amyloid precursor) |
| 457 | ubiquitin specific protease 9, Y-linked (fat facets-like, Drosophila) |
| 458 | ATP/GTP binding protein 1 |
| 459 | paraoxonase 2 |
| 460 | nucleosome assembly protein 1-like 1 |
| 461 | progesterone receptor membrane component 1 |
| 462 | CD58 antigen, (lymphocyte function-associated antigen 3) |
| 463 | CDC14 cell division cycle 14 homolog A (S. cerevisiae) |
| 464 | platelet factor 4 variant 1 |
| 465 | single-stranded DNA binding protein 1 |
| 466 | AG1 /// hypothetical protein DJ328E19.C1.1 /// hypothetical protein LOC200030 /// hypothetical protein MGC8902 |
| 467 | SH3 domain binding glutamic acid-rich protein like 3 /// SH3 domain binding glutamic acid-rich protein like 3 |
| 468 | poly(A) binding protein, nuclear 1 |
| 469 | epidermodysplasia verruciformis 1 |
| 470 | MRNA; cDNA DKFZp434A202 (from clone DKFZp434A202) |
| 471 | pleckstrin homology domain interacting protein |
| 472 | RUN and FYVE domain containing 1 |
| 473 | protein phosphatase 3 (formerly 2B), catalytic subunit, gamma isoform (calcineurin A gamma) |
| 474 | protein kinase, cAMP-dependent, regulatory, type I, alpha (tissue specific extinguisher 1) |
| 475 | chloride intracellular channel 4 |
| 476 | PHD finger protein 1 |
| 477 | protein kinase C, beta 1 |
| 478 | major histocompatibility complex, class II, DQ beta 1 |
| 479 | eukaryotic translation termination factor 1 |
| 480 | BCL2/adenovirus E1B 19 kDa interacting protein 3-like |
| 481 | arachidonate 12-lipoxygenase |
| 482 | chromosome 1 open reading frame 16 |
| 483 | solute carrier family 11 (proton-coupled divalent metal ion transporters), member 1 |
| 484 | RAB2, member RAS oncogene family |
| 485 | secreted protein, acidic, cysteine-rich (osteonectin) |
| 486 | N-acetyltransferase 8 (camello like) |
| 487 | dihydrolipoamide dehydrogenase (E3 component of pyruvate dehydrogenase complex, 2-oxo-glutarate complex, branched |
| | chain keto acid dehydrogenase complex) |
| 488 | cytochrome c oxidase subunit IV isoform 1 |
| 489 | HRAS-like suppressor |
| 490 | cyclin L1 |
| 491 | hemoglobin, alpha 2 |
| 492 | ariadne homolog 2 (Drosophila) |
| 493 | translocation associated membrane protein 1 |
| 494 | neural cell adhesion molecule 1 |
| 495 | SNF-1 related kinase |
| 496 | membrane-spanning 4-domains, subfamily A, member 4 |
| 497 | A kinase (PRKA) anchor protein 8-like |
| 498 | thiosulfate sulfurtransferase (rhodanese) |
| 499 | four and a half LIM domains 1 |
| 500 | microtubule-associated protein 1 light chain 3 beta |
| 501 | kinesin 2 60/70 kDa |
| 502 | chimerin (chimaerin) 2 |
| 503 | v-maf musculoaponeurotic fibrosarcoma oncogene homolog B (avian) |
| 504 | AT rich interactive domain 5B (MRF1-like) |
| 505 | prostaglandin-endoperoxide synthase 1 (prostaglandin G/H synthase and cyclooxygenase) |
| 506 | huntingtin interacting protein B |
| 507 | RAB11 family interacting protein 1 (class I) |
| 508 | — |
| 509 | oxidative-stress responsive 1 |
| 510 | SWAP-70 protein |
| 511 | elastase 2, neutrophil |
| 512 | RAB14, member RAS oncogene family |
| 513 | A kinase (PRKA) anchor protein (yotiao) 9 |
| 514 | KIAA1036 |
| 515 | EH-domain containing 3 |
| 516 | — |
| 517 | calmodulin 3 (phosphorylase kinase, delta) |
| 518 | asialoglycoprotein receptor 2 |
| 519 | phorbol-12-myristate-13-acetate-induced protein 1 |
| 520 | CD14 antigen |
| 521 | ribosomal protein L23 |
| 522 | SERTA domain containing 3 |
| 523 | myeloid/lymphoid or mixed-lineage leukemia 4 |
| 524 | cAMP responsive element binding protein 1 |
| 525 | pyruvate kinase, muscle |
| 526 | HCF-binding transcription factor Zhangfei |
| 527 | solute carrier family 16 (monocarboxylic acid transporters), member 3 |
| 528 | hypothetical protein FLJ22662 |
| 529 | zinc finger homeobox 1b |
| 530 | histone 1, H2ag |
| 531 | A kinase (PRKA) anchor protein 13 /// A kinase (PRKA) anchor protein 13 |
| 532 | colony stimulating factor 3 receptor (granulocyte) |
| 533 | major histocompatibility complex, class II, DQ beta 1 /// major histocompatibility complex, class II, DQ beta 1 |
| 534 | chemokine (C—X—C motif) receptor 4 |
| 535 | ATP citrate lyase |
| 536 | vesicle-associated membrane protein 3 (cellubrevin) |
| 537 | hypothetical protein FLJ11127 |
| 538 | Ras-like without CAAX 1 |
| 539 | Wolf-Hirschhom syndrome candidate 1 |
| 540 | heat shock 70 kDa protein 5 (glucose-regulated protein, 78 kDa) |
| 541 | ribonucleotide reductase M2 polypeptide |
| 542 | Rho-associated, coiled-coil containing protein kinase 2 |
| 543 | heterogeneous nuclear ribonucleoprotein D-like |
| 544 | solute carrier family 1 (glutamate/neutral amino acid transporter), member 4 |
| 545 | ATP-binding cassette, sub-family C (CFTR/MRP), member 3 |
| 546 | a disintegrin and metalloproteinase domain 9 (meltrin gamma) |
| 547 | nuclear factor of kappa light polypeptide gene enhancer in B-cells inhibitor, alpha |
| 548 | B-cell CLL/lymphoma 6 (zinc finger protein 51) |
| 549 | SWI/SNF related, matrix associated, actin dependent regulator of chromatin, subfamily a, member 2 |
| 550 | FUS interacting protein (serine-arginine rich) 1 |
| 551 | spermine oxidase |
| 552 | calnexin |
| 553 | MRNA full length insert cDNA clone EUROIMAGE 73337 |
| 554 | synovial sarcoma, X breakpoint 2 interacting protein |
| 555 | cyclin-dependent kinase 9 (CDC2-related kinase) |
| 556 | similar to bK246H3.1 (immunoglobulin lambda-like polypeptide 1, pre-B-cell specific) |
| 557 | chitinase 3-like 1 (cartilage glycoprotein-39) |
| 558 | fucosyltransferase 4 (alpha (1,3) fucosyltransferase, myeloid-specific) |
| 559 | ATPase, Ca++ transporting, plasma membrane 1 |
| 560 | tropomyosin 4 |
| 561 | SMC4 structural maintenance of chromosomes 4-like 1 (yeast) |
| 562 | N-acetylneuraminate pyruvate lyase (dihydrodipicolinate synthase) /// N-acetylneuraminate pyruvate lyase (dihydrodipicolinate |
| | synthase) |
| 563 | jumonji domain containing 2C |
| 564 | latent transforming growth factor beta binding protein 1 |
| 565 | prosaposin (variant Gaucher disease and variant metachromatic leukodystrophy) |
| 566 | — |
| 567 | DEAD (Asp-Glu-Ala-Asp) box polypeptide 58 |
| 568 | latent transforming growth factor beta binding protein 1 |
| 569 | colony stimulating factor 2 receptor, beta, tow-affinity (granulocyte-macrophage) |
| 570 | TcD37 homolog |
| 571 | caspase 1, apoptosis-related cysteine protease (interleukin 1, beta, convertase) |
| 572 | myosin, heavy polypeptide 9, non-muscle |
| 573 | cyclin-dependent kinase inhibitor 2D (p19, inhibits CDK4) |
| 574 | cathepsin K (pycnodysostosis) |
| 575 | cleavage and polyadenylation specific factor 1, 160 kDa |
| 576 | transforming growth factor beta 1 induced transcript 4 |
| 577 | FK506 binding protein 1A, 12 kDa |
| 578 | MAX interactor 1 |
| 579 | — |
| 580 | mitogen-activated protein kinase kinase 3 |
| 581 | mitogen-activated protein kinase kinase 5 |
| 582 | cullin 4A |
| 583 | amylase, alpha 2B; pancreatic |
| 584 | sorting nexin 2 |
| 585 | solute carrier family 24 (sodium/potassium/calcium exchanger), member 3 |
| 586 | dual specificity phosphatase 6 |
| 587 | v-yes-1 Yamaguchi sarcoma viral related oncogene homolog |
| 588 | SUMO1/sentrin/SMT3 specific protease 3 |
| 589 | KIAA0877 protein |
| 590 | PTK2B protein tyrosine kinase 2 beta |
| 591 | pentaxin-related gene, rapidly induced by IL-1 beta |
| 592 | solute carrier family 2 (facilitated glucose transporter), member 14 /// solute carrier family 2 (facilitated glucose transporter), |
| | member 3 |
| 593 | catenin (cadherin-associated protein), alpha-like 1 |
| 594 | ubiquitin-conjugating enzyme E2G 2 (UBC7 homolog, yeast) |
| 595 | proteasome (prosome, macropain) 26S subunit, non-ATPase, 11 |
| 596 | tyrosina 3-monooxygenase/tryptophan 5-monooxygenase activation protein, zeta polypeptide |
| 597 | chromosome 5 open reading frame 4 |
| 598 | golgi phosphoprotein 2 |
| 599 | splicing factor, arginine/serine-rich 15 |
| 600 | UDP-Gal:betaGlcNAc beta 1,4-galactosyltransferase, polypeptide 5 |
| 601 | cortactin |
| 602 | transmembrane 9 superfamily member 2 |
| 603 | H2A histone family, member V |
| 604 | 30 kDa protein |
| 605 | palladin |
| 606 | matrin 3 |
| 607 | SEC14-like 1 (S. cerevisiae) |
| 608 | SEC63-like (S. cerevisiae) |
| 609 | protein tyrosine phosphatase, receptor type, C |
| 610 | — |
| 611 | erythrocyte membrane protein band 4.1-like 3 |
| 612 | hypothetical protein FLJ22635 |
| 613 | toll-like receptor 2 |
| 614 | major histocompatibility complex, class II, DR alpha |
| 615 | ATP-binding cassette, sub-family C (CFTR/MRP), member 4 |
| 616 | spectrin repeat containing, nuclear envelope 2 |
| 617 | chromosome 14 open reading frame 138 |
| 618 | capping protein (actin filament), gelsolin-like |
| 619 | DnaJ (Hsp40) homolog, subfamily C, member 10 |
| 620 | general transcription factor IIIC, polypeptide 3, 102 kDa |
| 621 | cytidine deaminase |
| 622 | cathepsin S |
| 623 | chloride channel 3 |
| 624 | pannexin 1 |
| 625 | SWI/SNF related, matrix associated, actin dependent regulator of chromatin, subfamily c, member 2 |
| 626 | adaptor-related protein complex 2, mu 1 subunit |
| 627 | reticulon 1 |
| 628 | WW domain containing adaptor with coiled-coil |
| 629 | integrin, beta 3 (platelet glycoprotein IIIa, antigen CD61) |
| 630 | synaptojanin 2 |
| 631 | translin |
| 632 | CD47 antegen (Rh-related antigen, integrin-associated signal transducer) /// CD47 antigen (Rh-related antigen, integrin- |
| | associated signal transducer) |
| 633 | KIAA0794 protein |
| 634 | homogentisate 1,2-dioxygenase (homogentisate oxidase) |
| 635 | — |
| 636 | putative translation initiation factor |
| 637 | SH3-domain GRB2-like endophilin B1 |
| 638 | TcD37 homolog |
| 639 | pleckstrin |
| 640 | serine/threonine kinase 4 /// serine/threonine kinase 4 |
| 641 | decay accelerating factor for complement (CD55, Cromer blood group system) |
| 642 | T-cell leukemia/lymphoma 1A |
| 643 | DNA segment on chromosome X and Y (unique) 155 expressed sequence |
| 644 | O-linked N-acetylglucosamine (GlcNAc) transferase (UDP-N-acetylglucosamine:polypeptide-N-acetylglucosaminyl transferase) |
| 645 | mortality factor 4 like 1 |
| 646 | protein phosphatase 1, regulatory subunit 7 |
| 647 | splicing factor, arginine/serine-rich 12 |
| 648 | amyloid beta precursor protein binding protein 1, 59 kDa |
| 649 | meningioma expressed antigen 5 (hyaluronidase) |
| 650 | acyl-Coenzyme A dehydrogenase, very long chain |
| 651 | putative translation initiation factor |
| 652 | G protein-coupled receptor 56 |
| 653 | G-rich RNA sequence binding factor 1 |
| 654 | KIAA0252 |
| 655 | leukocyte membrane antigen |
| 656 | CCR4-NOT transcription complex, subunit 8 |
| 657 | hypothetical protein FLJ10808 |
| 658 | Ras-GTPase activating protein SH3 domain-binding protein 2 |
| 659 | — |
| 660 | hypothetical protein LOC54103 |
| 661 | NADH dehydrogenase (ubiquinone) 1 beta subcomplex, 8, 19 kDa |
| 662 | ADP-ribosylation factor 6 |
| 663 | chromosome 6 open reading frame 209 |
| 664 | vitamin D (1,25-dihydroxyvitamin D3) receptor |
| 665 | multiple C2-domains with two transmembrane regions 1 |
| 666 | UBX domain containing 2 |
| 667 | tudor domain containing 7 |
| 668 | UPF2 regulator of nonsense transcripts homolog (yeast) |
| 669 | splicing factor, arginine/serine-rich 4 |
| 670 | tubulin tyrosine ligase-like family, member 3 |
| 671 | CASP8 and FADD-like apoptosis regulator |
| 672 | development and differentiation enhancing factor 2 |
| 673 | CASP8 and FADD-like apoptosis regulator |
| 674 | phosphoglycerate kinase 1 |
| 675 | AMP-activated protein kinase family member 5 |
| 676 | likely ortholog of mouse acyl-Coenzyme A thioesterase 2, mitochondrial |
| 677 | aquaporin 9 |
| 678 | ATPase, Ca++ transporting, plasma membrane 4 |
| 679 | ATP-binding cassette, sub-family B (MDR/TAP), member 1 |
| 680 | filamin A, alpha (actin binding protein 280) |
| 681 | CD22 antigen |
| 682 | SCO cytochrome oxidase deficient homolog 2 (yeast) |
| 683 | TXK tyrosine kinase |
| 684 | hypothetical protein MGC3222 |
| 685 | Fc fragment of IgG, low affinity IIa, receptor for (CD32) |
| 686 | chromosome 20 open reading frame 67 |
| 687 | chromosome 21 open reading frame 4 |
| 688 | DNA-damage-inducible transcript 4 |
| 689 | RAB27A, member RAS oncogene family |
| 690 | spastic paraplegia 7, paraplegin (pure and complicated autosomal recessive) |
| 691 | CDC42 effector protein (Rho GTPase binding) 3 |
| 692 | ring finger protein 3 |
| 693 | UV radiation resistance associated gene |
| 694 | integrin, beta 3 (platelet glycoprotein IIIa, antigen CD61) |
| 695 | autism susceptibility candidate 2 |
| 696 | actinin, alpha 1 |
| 697 | transcription factor 15 (basic helix-loop-helix) |
| 698 | jun B proto-oncogene |
| 699 | electron-transfer-flavoprotein, alpha polypeptide (glutaric aciduria II) |
| 700 | nuclear RNA export factor 1 |
| 701 | ubiquitin-conjugating enzyme E2E3 (UBC4/5 homolog, yeast) |
| 702 | GABA(A) receptor-associated protein like 1 |
| 703 | phorbol-12-myristate-13-acetate-induced protein 1 |
| 704 | AHNAK nucleoprotein (desmoyokin) |
| 705 | serine/threonine kinase 24 (STE20 homolog, yeast) |
| 706 | retinoic acid induced 17 |
| 707 | nuclease sensitive element binding protein 1 |
| 708 | heme-regulated initiation factor 2-alpha kinase |
| 709 | CASP8 and FADD-like apoptosis regulator |
| 710 | stannin |
| 711 | zinc finger CCCH type domain containing 7 |
| 712 | Dicer1, Dcr-1 homolog (Drosophila) |
| 713 | solute carrier family 38, member 2 |
| 714 | histone 1, H2bk |
| 715 | UDP-N-acetyl-alpha-D-galactosamine:polypeptide N-acetylgalactosaminyltranaferase 10 (GalNAc-T10) |
| 716 | ATP-binding cassette, sub-family C (CFTR/MRP), member 3 |
| 717 | protein tyrosine phosphatase, receptor type, f polypeptide (PTPRF), interacting protein (liprin), alpha 1 |
| 718 | nuclear transcription factor Y, gamma |
| 719 | chromosome 19 open reading frame 13 |
| 720 | heterogeneous nuclear ribonucleoprotein H2 (H′) |
| 721 | family with sequence similarity 38, member A |
| 722 | interferon regulatory factor 4 |
| 723 | SKI interacting protein |
| 724 | hypothetical protein FLJ10996 |
| 725 | peroxisomal Ion protease |
| 726 | histone 1, H2bk |
| 727 | phosphoinositide-3-kinase, regulatory subunit, polypeptide 1 (p85 alpha) |
| 728 | retinoblastoma binding protein 4 |
| 729 | FYN binding protein (FYB-120/130) |
| 730 | fer-1-like 3, myoferlin (C. elegans) |
| 731 | kinesin heavy chain member 2 |
| 732 | ocular development-associated gene |
| 733 | Fc fragment of IgG, low affinity IIIa, receptor for (CD16) |
| 734 | — |
| 735 | non-POU domain containing, octamer-binding |
| 736 | DKFZP564G2022 protein |
| 737 | sel-1 suppressor of lin-12-like (C. elegans) |
| 738 | axotrophin |
| 739 | calcium binding protein Cab45 precursor |
| 740 | BTG family, member 3 |
| 741 | Ras and Rab interactor 3 |
| 742 | heat shock 70 kDa protein 4 |
| 743 | chromosome 20 open reading frame 67 |
| 744 | early growth response 1 |
| 745 | — |
| 746 | runt-related transcription factor 3 |
| 747 | DnaJ (Hsp40) homolog, subfamily B, member 1 |
| 748 | 5′,3′-nucleotidase, mitochondrial |
| 749 | coronin, actin binding protein, 1C |
| 750 | FOS-like antigen 2 |
| 751 | stabilin 1 |
| 752 | cytosolic ovarian carcinoma antigen 1 |
| 753 | folate receptor 3 (gamma) |
| 754 | Vpr-binding protein |
| 755 | neutrophil cytosolic factor 4, 40 kDa |
| 756 | spermidine/spermine N1-acetyltransferase |
| 757 | heme oxygenase (decycling) 1 |
| 758 | abI-interactor 1 |
| 759 | LIM and senescent cell antigen-like domains 1 |
| 760 | Rab geranylgeranyltransferase, beta subunit |
| 761 | KIAA0592 protein /// KIAA0592 protein |
| 762 | natural killer-tumor recognition sequence |
| 763 | SH3 domain containing, Ysc84-like 1 (S. cerevisiae) |
| 764 | epidermal growth factor (beta-urogastrone) |
| 765 | chloride channel 4 |
| 766 | spastic paraplegia 21 (autosomal recessive, Mast syndrome) |
| 767 | hypothetical protein FLJ32731 |
| 768 | myristoylated alanine-rich protein kinase C substrate |
| 769 | synovial sarcoma translocation gene on chromosome 18-like 1 |
| 770 | ring finger protein 11 |
| 771 | helicase with zinc finger domain |
| 772 | branched chain keto acid dehydrogenase E1, beta polypeptide (maple syrup urine disease) |
| 773 | disabled homolog 2, mitogen-responsive phosphoprotein (Drosophila) |
| 774 | insulin receptor |
| 775 | CDNA FLJ13876 fis, clone THYRO1001401 |
| 776 | lymphocyte cytosolic protein 1 (L-plastin) |
| 777 | leptin receptor gene-related protein |
| 778 | inositol 1,4,5-triphosphate receptor, type 1 |
| 779 | G protein-coupled receptor kinase interactor 2 |
| 780 | membrane-spanning 4-domains, subfamily A, member 1 |
| 781 | FYN oncogene related to SRC, FGR, YES |
| 782 | tribbles homolog 2 (Drosophila) |
| 783 | RING1 and YY1 binding protein |
| 784 | BCL2-related protein A1 |
| 785 | ubiquitin-conjugating enzyme E2, J1 (UBC6 homolog, yeast) |
| 786 | fumarylacetoacetate hydrolase (fumarylacetoacetase) |
| 787 | derlin-1 |
| 788 | nodal modulator 2 |
| 789 | CCAAT/enhancer binding protein (C/EBP), gamma |
| 790 | — |
| 791 | adiponectin receptor 1 |
| 792 | — |
| 793 | tousled-like kinase 1 |
| 794 | SET translocation (myeloid leukemia-associated) |
| 795 | KIAA0500 protein |
| 796 | leucine-rich repeats and immunoglobulin-like domains 2 |
| 797 | family with sequence similarity 3, member C |
| 798 | chondroitin sulfate GalNAcT-2 |
| 799 | nuclear factor of activated T-cells, cytoplasmic, calcineurin-dependent 3 |
| 800 | lamin A/C |
| 801 | disabled homolog 2, mitogen-responsive phosphoprotein (Drosophila) |
| 802 | tumor necrosis factor, alpha-induced protein 2 |
| 803 | chromosome 1 open reading frame 38 |
| 804 | Clone IMAGE: 5289004, mRNA |
| 805 | RNA binding motif protein 25 |
| 806 | TAF9 RNA polymerase II, TATA box binding protein (TBP)-associated factor, 32 kDa |
| 807 | aldehyde dehydrogenase 2 family (mitochondrial) |
| 808 | zinc finger protein 224 |
| 809 | nucleobindin 2 |
| 810 | DR1-associated protein 1 (negative cofactor 2 alpha) |
| 811 | chromosome 14 open reading frame 147 |
| 812 | GRB2-related adaptor protein 2 |
| 813 | RNA-binding protein |
| 814 | — |
| 815 | LOC387866 |
| 816 | — |
| 817 | microtubule-associated protein, RP/EB family, member 2 |
| 818 | RAN binding protein 2 |
| 819 | protein tyrosine phosphatase type IVA, member 2 |
| 820 | SMA5 |
| 821 | SRY (sex determining region Y)-box 4 |
| 822 | soc-2 suppressor of clear homolog (C. elegans) |
| 823 | ceroid-lipofuscinosis, neuronal 2, late infantile (Jansky-Bielschowsky disease) |
| 824 | KIAA0513 gene product |
| 825 | protein phosphatase 1, regulatory (inhibitor) subunit 2 |
| 826 | down-regulator of transcription 1, TBP-binding (negative cofactor 2) |
| 827 | KDEL (Lys-Asp-Glu-Leu) endoplasmic reticulum protein retention receptor 2 |
| 828 | ATP-binding cassette, sub-family B (MDR/TAP), member 1 /// ATP-binding cassette, sub-family B (MDR/TAP), member 4 |
| 829 | haptoglobin |
| 830 | death-associated protein kinase 1 |
| 831 | nuclear factor of activated T-cells, cytoplasmic, calcineurin-dependent 2 interacting protein |
| 832 | adenylate kinase 1 |
| 833 | KIAA0240 |
| 834 | zyxin |
| 835 | tubby like protein 4 |
| 836 | patatin-like phospholipase domain containing 2 |
| 837 | adducin 1 (alpha) |
| 838 | flightless I homolog (Drosophila) |
| 839 | disabled homolog 2, mitogen-responsive phosphoprotein (Drosophila) /// CDNA FLJ35517 fis, clone SPLEN2000698 |
| 840 | A kinase (PRKA) anchor protein 11 |
| 841 | interleukin-1 receptor-associated kinase 3 |
| 842 | lipopolysaccharide-induced TNF factor |
| 843 | activin A receptor, type II |
| 844 | fem-1 homolog b (C. elegans) |
| 845 | eukaryotic translation initiation factor 3, subunit 10 theta, 150/170 kDa |
| 846 | immunoglobulin lambda-like polypeptide 1 |
| 847 | eukaryotic translation initiation factor 2, subunit 3 gamma, 52 kDa |
| 848 | transforming growth factor, beta-induced, 68 kDa |
| 849 | heat shock 70 kDa protein 9B (mortalin-2) |
| 850 | flightless I homolog (Drosophila) |
| 851 | hypothetical protein FLJ10996 |
| 852 | myotubularin related protein 3 |
| 853 | phosphorylase, glycogen; liver (Hers disease, glycogen storage disease type VI) |
| 854 | plectin 1, intermediate filament binding protein 500 kDa |
| 855 | phospholipase A2, group IVC (cytosolic, calcium-independent) |
| 856 | vascular endothelial growth factor |
| 857 | RAB11A, member RAS oncogene family |
| 858 | protein kinase, X-linked |
| 859 | phospholipase C, beta 1 (phosphoinositide-specific) |
| 860 | SIN3 homolog B, transcriptional regulator (yeast) |
| 861 | COX11 homolog, cytochrome c oxidase assembly protein (yeast) /// COX11 homolog, cytochrome c oxidase assembly protein |
| | (yeast) |
| 862 | RNA binding motif protein 5 |
| 863 | transforming growth factor beta 1 induced transcript 1 |
| 864 | androgen-induced proliferation inhibitor |
| 865 | SMT3 suppressor of mif two 3 homolog 1 (yeast) |
| 866 | rab3 GTPase-activating protein, non-catalytic subunit (150 kD) |
| 867 | Rho-related BTB domain containing 1 |
| 868 | ubiquitin-conjugating enzyme E2, J1 (UBC6 homolog, yeast) |
| 869 | nuclear receptor co-repressor 2 |
| 870 | villin 2 (ezrin) |
| 871 | erythropoietin receptor |
| 872 | Jumonji, AT rich interactive domain 1B (RBP2-like) |
| 873 | DIP13 beta |
| 874 | protein tyrosine phosphatase, non-receptor type 12 |
| 875 | similar to S. cerevisiae SSM4 |
| 876 | reticulon 3 |
| 877 | a disintegrin and metalloproteinase domain 17 (tumor necrosis factor, alpha, converting enzyme) |
| 878 | SMC6 structural maintenance of chromosomes 6-like 1 (yeast) |
| 879 | limkain b1 |
| 880 | ribosomal protein L37a |
| 881 | collagen, type IX, alpha 3 |
| 882 | phosphoglycerate kinase 1 |
| 883 | quaking homolog, KH domain RNA binding (mouse) |
| 884 | sulfotransferase family, cytosolic, 1A, phenol-preferring, member 3 |
| 885 | serine (or cysteine) proteinase inhibitor, clade B (ovalbumin), member 1 |
| 886 | hypothetical protein MGC5306 |
| 887 | hypothetical protein BC002926 |
| 888 | diaphanous homolog 2 (Drosophila) |
| 889 | chromosome 21 open reading frame 7 |
| 890 | CCAAT/enhancer binding protein (C/EBP), delta |
| 891 | intersectin 2 |
| 892 | lipopolysaccharide-induced TNF factor |
| 893 | poly (ADP-ribose) polymerase family, member 8 |
| 894 | membrane-spanning 4-domains, subfamily A, member 1 |
| 895 | hypothetical protein LOC153561 |
| 896 | hypothetical protein CL640 |
| 897 | glycoprotein Ib (platelet), alpha polypeptide |
| 898 | immunoglobulin heavy constant gamma 1 (G1m marker) /// immunoglobulin heavy constant gamma 1 (G1m marker) |
| 899 | protein phosphatase 3 (formerly 2B), catalytic subunit, alpha isoform (calcineurin A alpha) |
| 900 | heterogeneous nuclear ribonucleoprotein H3 (2H9) |
| 901 | — |
| 902 | forkhead box O3A |
| 903 | interleukin 13 receptor, alpha 1 |
| 904 | KIAA0226 gene product |
| 905 | protein kinase, Y-linked |
| 906 | quaking homolog, KH domain RNA binding (mouse) |
| 907 | protein kinase C, theta |
| 908 | tensin |
| 909 | SWAP-70 protein |
| 910 | TBC1 domain family, member 8 (with GRAM domain) |
| 911 | cytochrome b-245, beta polypeptide (chronic granulomatous disease) |
| 912 | secemin 1 |
| 913 | MRNA; cDNA DKFZp586E2317 (from clone DKFZp586E2317) |
| 914 | regulator of G-protein signalling 1 |
| 915 | chromosome 17 open reading frame 25 |
| 916 | annexin A4 |
| 917 | cell division cycle 2-like 1 (PITSLRE proteins) |
| 918 | RIO kinase 3 (yeast) |
| 919 | RAB13, member RAS oncogene family |
| 920 | presenilin 1 (Alzheimer disease 3) |
| 921 | glutaminyl-peptide cyclotransferase (glutaminyl cyclase) |
| 922 | DnaJ (Hsp40) homolog, subfamily B, member 6 |
| 923 | staufen, RNA binding protein (Drosophila) |
| 924 | caspase 1, apoptosis-related cysteine protease (interleukin 1, beta, convertase) |
| 925 | WD40 repeat protein Interacting with phosphoinositides of 49 kDa |
| 926 | microtubule-actin crosslinking factor 1 |
| 927 | nucleosome assembly protein 1-like 1 |
| 928 | sorting nexin 4 |
| 929 | glycogenin |
| 930 | hypothetical protein FLJ11822 |
| 931 | replication protein A1, 70 kDa |
| 932 | zinc finger protein 394 |
| 933 | leucine rich repeat (in FLII) interacting protein 2 |
| 934 | SAR1a gene homolog 1 (S. cerevisiae) |
| 935 | insulin induced gene 1 |
| 936 | hypothetical protein LOC54103 |
| 937 | KIAA1219 protein |
| 938 | catenin (cadherin-associated protein), alpha 1, 102 kDa |
| 939 | Friend leukemia virus integration 1 |
| 940 | DEAD (Asp-Glu-Ala-Asp) box polypeptide 3, X-linked |
| 941 | solute carrier family 11 (proton-coupled divalent metal ion transporters), member 2 |
| 942 | B-cell linker |
| 943 | F-box and leucine-rich repeat protein 14 |
| 944 | transcription factor-like 5 (basic helix-loop-helix) |
| 945 | S-phase kinase-associated protein 1A (p19A) |
| 946 | mitochondrial solute carrier protein |
| 947 | solute carrier family 25, member 28 /// solute carrier family 25, member 28 |
| 948 | Jumonji, AT rich interactive domain 1D (RBP2-like) |
| 949 | Sec23 homolog B (S. cerevisiae) |
| 950 | stomatin |
| 951 | kynureninase (L-kynurenine hydrolase) |
| 952 | embryonic ectoderm development |
| 953 | TL132 protein |
| 954 | NCK adaptor protein 1 |
| 955 | inositol polyphosphate-4-phosphatase, type I, 107 kDa |
| 956 | — |
| 957 | SMA3 |
| 958 | golgin-67 |
| 959 | nucleoporin 205 kDa |
| 960 | metastasis associated 1 /// metastasis associated 1 |
| 961 | SMAD specific E3 ubiquilin protein ligase 1 |
| 962 | solute carrier family 2 (facilitated glucose transporter), member 3 |
| 963 | filamin A, alpha (actin binding protein 280) |
| 964 | immunoglobulin superfamily, member 4 |
| 965 | Ras-GTPase activating protein SH3 domain-binding protein 2 |
| 966 | growth arrest-specific 2 like 1 |
| 967 | major histocompatibility complex, class II, DP alpha 1 |
| 968 | carcinoembryonic antigen-related cell adhesion molecule 6 (non-specific cross reacting antigen) /// carcinoembryonic antigen- |
| | related cell adhesion molecule 6 (non-specific cross reacting antigen) |
| 969 | hypothetical protein FLJ10134 |
| 970 | splicing factor 1 |
| 971 | nuclear factor of kappa light polypeptide gene enhancer in B-cells 2 (p49/p100) |
| 972 | hect domain and RLD 2 |
| 973 | endothelial cell growth factor 1 (platelet-derived) |
| 974 | azurocidin 1 (cationic antimicrobial protein 37) |
| 975 | F-box and leucine-rich repeat protein 5 |
| 976 | lectin, galactoside-binding, soluble, 2 (galectin 2) |
| 977 | coagulation factor XIII, A1 polypeptide |
| 978 | — |
| 979 | PDZ and LIM domain 1 (elfin) |
| 980 | T-cell leukemia/lymphoma 1A |
| 981 | kynureninase (L-kynurenine hydrolase) |
| 982 | cleavage and polyadenylation specific factor 1, 160 kDa) |
| 983 | aminopeptidase-like 1 |
| 984 | lymphocyte cytosolic protein 2 (SH2 domain containing leukocyte protein of 76 kDa) |
| 985 | aquarius homolog (mouse) |
| 986 | galactokinase 2 |
| 987 | NAD(P)H: quinone oxidoreductase type 3, polypeptide A2 |
| 988 | G1 to S phase transition 1 |
| 989 | — |
| 990 | pellino homolog 2 (Drosophila) |
| 991 | eukaryotic translation initiation factor 5 |
| 992 | CSE1 chromosome segregation 1-like (yeast) |
| 993 | ribonucleotide reductase M2 polypeptide |
| 994 | complement component 5 receptor 1 (C5a ligand) |
| 995 | ornithine decarboxylase antizyme inhibitor |
| 996 | adenosylmethionine decarboxylase 1 |
| 997 | KIAA0182 protein 1 |
| 998 | nerve growth factor receptor (TNFRSF16) associated protein 1 |
| 999 | UDP-Gal:betaGlcNAc beta 1,4-galactosyltransferase, polypeptide 5 |
| 1000 | cylindromatosis(turban tumor syndrome) |
| 1001 | CD44 antigen (homing function and Indian blood group system) |
| 1002 | solute carrier family 38, member 2 |
| 1003 | A kinase(PRKA) anchor protein 13 |
| 1004 | peroxiredoxin 3 |
| 1005 | ring finger protein 14 |
| 1006 | REST corepressor 1 |
| 1007 | galactosidase, alpha |
| 1008 | hypothetical protein MGC27165 |
| 1009 | actin binding LIM protein 1 |
| 1010 | KIAA0102 gene product |
| 1011 | WD repeat and FYVE domain containing 3 |
| 1012 | papillary renal cell carcinoma (translocation-associated) |
| 1013 | UDP-N-acetyl-alpha-D-galactosamine:polypeptide N-acetylgalactosaminyltransferase 10 (GalNAc-T10) |
| 1014 | IQ motif and Sec7 domain 1 |
| 1015 | NADH dehydrogenase (ubiquinone) flavoprotain 2, 24 kDA |
| 1016 | — |
| 1017 | golgi autoantigen, golgin aubfamily a, 4 |
| 1018 | KIAA0232 gene product |
| 1019 | family with sequence similarity 13, member A1 |
| 1020 | HSPC159 protein |
| 1021 | proteasome (prosome, macropain) subunit, alpha type, 1 |
| 1022 | GA binding protein transcription factor, beta subunit 2, 47 kDa |
| 1023 | POU domain, class 2, associating factor 1 |
| 1024 | SON DNA binding protein |
| 1025 | insulin receptor subtrate 2 |
| 1026 | receptor TNFRSF)-interacting serine-threonine kinase 1 |
| 1027 | dipeptidase 2 |
| 1028 | FCH and double SH3 domains 2 |
| 1029 | plexin B2 |
| 1030 | protein kinase N1 |
| 1031 | eukaryotic translation initiation factor 4E |
| 1032 | nuclear factor of activated T-cells, cytoplasmic, calcineurin-dependent 2 interacting protein |
| 1033 | annexin A4 |
| 1034 | peptidylprolyl isomerase F (cyclophilin F) |
| 1035 | RNA binding motif (RNP1, RRM)protein 3 |
| 1036 | — |
| 1037 | desmuslin |
| 1038 | — |
| 1039 | C1q domain containing 1 |
| 1040 | heat shock 60 kDa protein 1 (chaperonin) |
| 1041 | early growth response 3 |
| 1042 | PRP3 pre-mRNA processing factor 3 homolog (yeast) |
| 1043 | zinc finger protein 318 |
| 1044 | toll-like receptor 4 |
| 1045 | COP9 constitutive photomorphogenic homolog subunit 3 (Arabidopsis) |
| 1046 | programmed cell death 4 (neoplastic transformation inhibitor) |
| 1047 | KIAA1033 protein |
| 1048 | phospholipid scramblase 1 |
| 1049 | hypothetical protein FLJ10099 |
| 1050 | transgelin 2 |
| 1051 | ceroid-lipofuscinosis, neuronal 2, late infantile (Jansky-Bielschowsky disease) |
| 1052 | hypothetical protein FLJ13149 |
| 1053 | — |
| 1054 | CD58 antigen, (lymphocyte function-associated antigen 3) |
| 1055 | RAR-related orphan receptor A |
| 1056 | Clone 23872 mRNA seqence |
| 1057 | hypothetical protein SP192 |
| 1058 | ras-related C3 botulinum toxin substrate 2 (rho family, small GTP binding protein Rac2) |
| 1059 | chromosome 5 open reading frame 13 |
| 1060 | carboxypeptidase M |
| 1061 | tangerin |
| 1062 | ABC blood group (transferase A, alpha 1-3-N-acetylgalactosaminyltransferase; transfarase B, alpha 1-3-galactosyltransferase) |
| 1063 | peroxisome biogenesis factor 1 |
| 1064 | leukocyte immunoglobulin-like receptor, subfamily B (with TM and ITIM domains), member 2 |
| 1065 | adrenomedullin |
| 1066 | lectin, galactoside-binding, soluble, 8 (galectin 8) |
| 1067 | hypothetical protein MGC27165 /// immunoglobulin heavy constant gamma 1 (G1m marker) |
| 1068 | calnexin |
| 1069 | Hermansky-Pudlak syndrome 4 |
| 1070 | spectrin repeat containing, nuclear envelope 1 |
| 1071 | calmodulin 1 (phosphorylase kinase, delta) |
| 1072 | diacylglycerol kinase, alpha 80 kDa |
| 1073 | vaccinia related kinase 1 |
| 1074 | chromosome 21 open reading frame 59 |
| 1075 | KIAA0217 |
| 1076 | putative breast adenocarcinoma marker (32 kD) |
| 1077 | scaffold attachment factor B2 |
| 1078 | mannosyl (alpha-1,3-)-glycoprotein beta-1,4-N-acetylglucosaminyltransferase, isoenzyme A |
| 1079 | outer dense fiber of sperm tails 2 |
| 1080 | splicing actor,arginine/serine-rich 10 (transformer 2 homolog, Drosophila) |
| 1081 | pleckstrin homology domain containing, family A member 5 |
| 1082 | transcription factor 4 |
| 1083 | transcription factor Dp-1 |
| 1084 | caspase 4, apoptosis-related cysteine protease |
| 1085 | protein kinase C substrate 80K-H |
| 1086 | farnesyl-diphosphate farnesyltransferease 1 |
| 1087 | jumonji domain containing 1B |
| 1088 | phosphatase and actin regulator 2 |
| 1089 | nuclear protein double minute 1 |
| 1090 | granulysin |
| 1091 | suppressor of var1, 3-like 1 (S. cerevisiae) |
| 1092 | collagen, type VI, alpha 3 |
| 1093 | synovial sarcoma, X breakpoint 2 interacting protein |
| 1094 | G patch domain containing 2 |
| 1095 | leukocyte immunoglobulin-like receptor, subfamily A (with TM domain), member 2 |
| 1096 | EGF-like-domain, multiple 5 |
| 1097 | CD37 antigen |
| 1098 | CD59 antigen p18-20 (antigen identified by monoclonal antibodies 16.3A5, EJ16, EJ30, EL32 and G344) |
| 1099 | — |
| 1100 | ubiquitin protein ligase E3B |
| 1101 | KIAA1055 protein |
| 1102 | F-box and leucine-rich repeat protein 6 |
| 1103 | v-abl Abelson murine leukemia viral oncogene homolog 1 |
| 1104 | tuberous sclerosis 2 |
| 1105 | tryptophanyl-tRNA synthetase |
| 1106 | nuclear transcription factor Y, gamma |
| 1107 | fem-1 homolog b (C. elegans) |
| 1108 | cyclin G2 |
| 1109 | cytochrome b reductase 1 |
| 1110 | signal transducer and activator of transcription 3 (acute-phase response factor) |
| 1111 | beclin 1 (coiled-coil, myosin-like BCL2 interacting protein) |
| 1112 | SRY (sex determining region Y)-box 4 |
| 1113 | F-box protein 7 |
| 1114 | — |
| 1115 | — |
| 1116 | PI-3-kinase-related kinase SMG-1-like |
| 1117 | cleavage and polyadenylation specific factor 6, 68 kDa |
| 1118 | lipid phosphate phosphatase-related protein type 2 |
| 1119 | sec1 family domain containing 1 |
| 1120 | erythrocyte membrane protein band 4.2 |
| 1121 | — |
| 1122 | kelch-like 2, Mayven (Drosophila) |
| 1123 | Rap guanine nucleotide exchange factor (GEF) 2 |
| 1124 | — |
| 1125 | Fc fragment of IgE, high affinity I, receptor for; alpha polypeptide /// Fc fragment of IgE, high affinity I, receptor for; alpha |
| | polypeptide |
| 1126 | hepatocellular carcinoma-associated antigen 112 |
| 1127 | ATP-binding cassette, sub-family A (ABC1), member 5 |
| 1128 | mannosidase, alpha, class 2A, member 2 |
| 1129 | KIAA0870 protein |
| 1130 | chemokine (C-C motif) receptor 1 |
| 1131 | zinc finger, BED domain containing 4 |
| 1132 | KIAA0746 protein |
| 1133 | nuclear autoantigenic sperm protein (histone-binding) |
| 1134 | IQ motif containing GTPase activating protein 1 |
| 1135 | FLJ41352 protein /// hypothetical protein FLJ20297 |
| 1136 | AT rich interactive domain 1A (SWI-like) |
| 1137 | nuclear transcription factor Y, alpha |
| 1138 | dishevelled, dsh homolog 3 (Drosophila) |
| 1139 | paxillin |
| 1140 | DNA fragmentation factor 40 kDa, beta polypeptide (caspase-activated DNase) |
| 1141 | ariadne homolog, ubiquitin-conjugating enzyme E2 binding protein, 1 (Drosophila) |
| 1142 | GPI deacylase |
| 1143 | makorin, ring finger protein, 1 |
| 1144 | — |
| 1145 | sphingomyelin phosphodiesterase, acid-like 3A |
| 1146 | mitogen-activated protein kinase kinase kinase 7 |
| 1147 | speckle-type POZ protein |
| 1148 | Rho GTPase-activating protein |
| 1149 | major histocompatibility complex, class I, C /// major histocompatibility complex, class I, C |
| 1150 | ADP-ribosylation factor 4 |
| 1151 | superoxide dismutase 2, mitochondrial |
| 1152 | hypothetical protein FLJ13855 |
| 1153 | zinc finger, A20 domain containing 2 |
| 1154 | tensin-like SH2 domain containing 1 |
| 1155 | Similar to bA110H4.2 (similar to membrane protein) |
| 1156 | chromosome 20 open reading frame 111 |
| 1157 | solute carrier family 7 (cationic amino acid transporter, y+ system), member 7 |
| 1158 | voltage-dependent anion channel 3 |
| 1159 | diacylglycerol kinase, alpha 80 kDa |
| 1160 | KIAA0669 gene product |
| 1161 | zinc finger protein 36, C3H type-like 2 |
| 1162 | nephronophthisis 4 |
| 1163 | multiple coagulation factor deficiency 2 |
| 1164 | thromboxane A synthase 1 (platelet, cytochrome P450, family 5, subfamily A) /// thromboxane A synthase 1 (platelet, |
| | cytochrome P450, family 5, subfamily A) |
| 1165 | solute carrier family 39 (zinc transporter), member 6 |
| 1166 | SEC24 related gene family, member D (S. cerevisiae) |
| 1167 | mitogen-activated protein kinase-activated protein kinase 5 |
| 1168 | — |
| 1169 | pleckstrin and Sec7 domain containing 3 |
| 1170 | solute carrier family 9 (sodium/hydrogen exchanger), isoform 6 |
| 1171 | nuclear protein double minute 1 |
| 1172 | PC2 (positive cofactor 2, multiprotein complex) glutamine/Q-rich-associated protein |
| 1173 | glutaminase |
| 1174 | SP140 nuclear body protein |
| 1175 | EF-hand domain (C-terminal) containing 1 |
| 1176 | interleukin 1, beta |
| 1177 | zinc finger protein 207 |
| 1178 | hypothetical protein FLJ14153 |
| 1179 | polo-like kinase 3 (Drosophila) |
| 1180 | interleukin 1, beta |
| 1181 | NACHT, leucine rich repeat and PYD containing 1 |
| 1182 | nuclear transcription factor Y, gamma |
| 1183 | KIAA1128 |
| 1184 | protein phosphatase 3 (formerly 2B), catalytic subunit, alpha isoform (calcineurin A alpha) |
| 1185 | chromosome 14 open reading frame 139 |
| 1186 | platelet factor 4 (chemokine (C—X—C motif) ligand 4) |
| 1187 | phosphodiesterase 4B, cAMP-specific (phosphodiesterase E4 dunce homolog, Drosphila) |
| 1188 | capping protein (actin filament) muscle Z-line, alpha 2 |
| 1189 | amyloid beta (A4) precursor protein-binding, family B, member 1 interacting protein |
| 1190 | mitogen-activated protein kinase kinase 7 |
| 1191 | proteasome (prosome, macropain) activator subunit 4 |
| 1192 | A kinase (PRKA) anchor protein (gravin) 12 |
| 1193 | Kruppel-like factor 4 (gut) |
| 1194 | selenium binding protein 1 |
| 1195 | LIM and senescent cell antigen-like domains 1 |
| 1196 | RAB27A, member RAS oncogene family |
| 1197 | phosphodiestearase 4D interacting protein (myomegalin) |
| 1198 | synaptosomal-associated protein, 23 kDa |
| 1199 | RAR-related orphan receptor A |
| 1200 | DnaJ (Hsp40) homolog, subfamily B, member 1 |
| 1201 | tumor protein D52 |
| 1202 | hemopoietic cell kinase |
| 1203 | septin 10 |
| 1204 | BCL2-like 1 |
| 1205 | protein phosphatase 1, regulatory (inhibitor) subunit 15A |
| 1206 | prostate tumor overexpressed gene 1 |
| 1207 | putative NFkB activating protein 373 |
| 1208 | KIAA0247 |
| 1209 | capping protein (actin filament) muscle Z-line, alpha 2 |
| 1210 | O-linked N-acetylglucosamine (GlcNAc) transferase (UDP-N-acetylglucosamine:polypeptide-N-acetylglucosaminyl transferase) |
| 1211 | unc-51-like kinase 1 (C. elegans) |
| 1212 | X-ray repair complementing defective repair in Chinese hamster cells 5 (double-strand-break rejoining; Ku autoantigen, 80 kDa) |
| 1213 | jun D proto-oncogene |
| 1214 | lipin 2 |
| 1215 | O-linked N-acetylglucosamine (GlcNAc) transferase (UDP-N-acetylglucosamine:polypeptide-N-acetylglucosaminyl transferase) |
| 1216 | cyclin-dependent kinase inhibitor 1C (p57, Kip2) |
| 1217 | Tara-like protein |
| 1218 | MAX dimerization protein 1 |
| 1219 | amyloid beta (A4) precursor-like protein 2 |
| 1220 | pogo transposable element with KRAB domain |
| 1221 | sorting nexin 3 |
| 1222 | WD repeat domain 26 |
| 1223 | DRE1 protein |
| 1224 | zinc finger protein 83 (HPF1) |
| 1225 | putative translation initiation factor |
| 1226 | nebulin |
| 1227 | receptor associated protein 80 |
| 1228 | GABA(A) receptor-associated protein like 1 |
| 1229 | scaffold attachment factor B |
| 1230 | WW domain binding protein 2 |
| 1231 | GCN1 general control of amino-acid synthesis 1-like 1 (yeast) |
| 1232 | alveolar soft part sarcoma chromosome region, candidate 1 |
| 1233 | cytoplasmic linker associated protein 2 |
| 1234 | kinesin family member 1B |
| 1235 | Janus kinase 2 (a protein tyrosine kinase) |
| 1236 | protein kinase C, eta |
| 1237 | annexin A1 |
| 1238 | heterogeneous nuclear ribonucleoprotein H3 (2H9) |
| 1239 | mitogen-activated protein kinase kinase kinase 7 |
| 1240 | solute carrier family 7 (cationic amino acid transporter, y+ system), member 5 |
| 1241 | zinc finger protein 148 (pHZ-52) |
| 1242 | actin related protein 2/3 complex, subunit 4, 20 kDa /// actin related protein 2/3 complex, subunit 4, 20 kDa |
| 1243 | WW domain containing adaptor with coiled-coil |
| 1244 | phosphotriesterase related |
| 1245 | RAB22A, member RAS oncogene family |
| 1246 | U2-associated SR140 protein |
| 1247 | Rap guanine nucleotide exchange factor (GEF) 2 |
| 1248 | activating signal cointegrator 1 complex subunit 2 |
| 1249 | dehydrogenase/reductase (SDR family) member 7 |
| 1250 | zinc finger and BTB domain containing 1 |
| 1251 | diphtheria toxin receptor (heparin-binding epidermal growth factor-like growth factor) |
| 1252 | phosphatase and actin regulator 2 |
| 1253 | OGT(O-Glc-NAc transferase)-interacting protein 106 KDa |
| 1254 | cytochrome P450, family 20, subfamily A, polypeptide 1 |
| 1255 | profilin 1 |
| 1256 | — |
| 1257 | nuclear receptor interacting protein 1 |
| 1258 | ceroid-lipofuscinosis, neuronal 3, juvenile (Batten, Spielmeyer-Vogt disease) |
| 1259 | chromosome 6 open reading frame 133 |
| 1260 | cyclin-dependent kinase inhibitor 1C (p57, Kip2) |
| 1261 | apoptotic chromatin condensation inducer 1 |
| 1262 | N-glycanase 1 |
| 1263 | platelet-activating factor receptor /// platelet-activating factor receptor |
| 1264 | ubiquitin specific protease 52 |
| 1265 | hypothetical protein MGC11349 |
| 1266 | KIAA1641 |
| 1267 | myosin XVB, pseudogene |
| 1268 | hypothetical protein DKFZp434B044 |
| 1269 | General transcription factor IIH, polypeptide 2, 44 kDa |
| 1270 | nucleoporin 210 |
| 1271 | aminopeptidase puromycin sensitive |
| 1272 | NADH dehydrogenase (ubiquinone) Fe—S protein 1, 75 kDa (NADH-coenzyme Q reductase) |
| 1273 | trans-prenyltransferase |
| 1274 | polymerase (DNA directed) iota |
| 1275 | nuclear receptor subfamily 2, group C, member 1 |
| 1276 | ral guanine nucleotide dissociation stimulator |
| 1277 | intercellular adhesion molecule 1 (CD54), human rhinovirus receptor |
| 1278 | type 1 tumor necrosis factor receptor shedding aminopeptidase regulator |
| 1279 | arginyl aminopeptidase (aminopeptidase B) |
| 1280 | — |
| 1281 | cyclin-dependent kinase inhibitor 1C (p57, Kip2) |
| 1282 | T cell receptor delta diversity 3 /// T cell receptor delta locus |
| 1283 | staufen, RNA binding protein (Drosophila) |
| 1284 | BTB (POZ) domain containing 3 |
| 1285 | adenosine kinase |
| 1286 | signal sequence receptor, alpha (translocon-associated protein alpha) |
| 1287 | brain expressed X-linked-like 1 |
| 1288 | jumonji domain containing 1A |
| 1289 | checkpoint suppressor 1 |
| 1290 | ATPase, Cu++ transporting, alpha polypeptide (Menkes syndrome) |
| 1291 | toll-like receptor 1 |
| 1292 | KIAA0650 protein |
| 1293 | CGI-49 protein |
| 1294 | inositol 1,4,5-triphosphate receptor, type 3 |
| 1295 | RAB8A, member RAS oncogene family |
| 1296 | ring finger protein 139 |
| 1297 | BH3 interacting domain death agonist /// BH3 interacting domain death agonist |
| 1298 | adaptor-related protein complex 1, beta 1 subunit |
| 1299 | EF hand domain containing 2 |
| 1300 | — |
| 1301 | amyloid beta (A4) precursor-like protein 2 |
| 1302 | Rho GTPase activating protein 26 |
| 1303 | ankyrin repeat domain 25 |
| 1304 | p21/Cdc42/Rac1-activated kinase 1 (STE20 homolog, yeast) |
| 1305 | hypothetical protein FLJ12750 /// hypothetical protein FLJ12750 |
| 1306 | Jumonji, AT rich interactive domain 2 |
| 1307 | suppressor of Ty 4 homolog 1 (S. cerevisiae) |
| 1308 | formin homology 2 domain containing 1 |
| 1309 | regulator of G-protein signalling 10 |
| 1310 | — |
| 1311 | CD44 antigen (homing function and Indian blood group system) |
| 1312 | killer cell lectin-like receptor subfamily C, member 3 |
| 1313 | synaptotagmin binding, cytoplasmic RNA interacting protein |
| 1314 | inhibitor of DNA binding 2, dominant negative helix-loop-helix protein |
| 1315 | ubiquitin associated protein 2-like |
| 1316 | erythrocyte membrane protein band 4.1-like 2 |
| 1317 | — |
| 1318 | engulfment and cell motility 1 (ced-12 homolog, C. elegans) |
| 1319 | transferrin receptor (p90, CD71) |
| 1320 | SMAD, mothers against DPP homolog 1 (Drosophila) |
| 1321 | spermine synthase |
| 1322 | chromosome 9 open reading frame 55 |
| 1323 | lymphoid-restricted membrane protein |
| 1324 | USP6 N-terminal like |
| 1325 | histone 1, H2bj |
| 1326 | TAR DNA binding protein /// TAR DNA binding protein |
| 1327 | polymerase (DNA directed), gamma |
| 1328 | tripartite motif-containing 33 |
| 1329 | tumor necrosis factor receptor superfamily, member 6 |
| 1330 | ATPase, H+ transporting, lysosomal 38 kDa, V0 subunit d isoform 1 |
| 1331 | calpain 1, (mu/l) large subunit |
| 1332 | LOC388483 |
| 1333 | ectonucleoside triphosphate diphosphohydrolase 1 |
| 1334 | dual specificity phosphatase 5 |
| 1335 | salvador homolog 1 (Drosophila) |
| 1336 | HSPB (heat shock 27 kDa) associated protein 1 |
| 1337 | myomesin (M-protein) 2, 165 kDa |
| 1338 | insulin receptor substrate 1 |
| 1339 | catenin (cadherin-associated protein), alpha 1, 102 kDa |
| 1340 | lymphoid-restricted membrane protein |
| 1341 | tumor protein D52 |
| 1342 | KIAA0563 gene product |
| 1343 | hypoxia up-regulated 1 |
| 1344 | praja 2, RING-H2 motif containing |
| 1345 | ATP-binding cassette, sub-family F (GCN20), member 2 |
| 1346 | unc-51-like kinase 2 (C. elegans) |
| 1347 | KIAA0073 protein |
| 1348 | — |
| 1349 | B-cell CLL/lymphoma 11B (zinc finger protein) |
| 1350 | lysosomal-associated membrane protein 1 |
| 1351 | Rho-associated, coiled-coil containing protein kinase 1 |
| 1352 | splicing factor, arginine/serine-rich 3 |
| 1353 | carbonic anhydrase I |
| 1354 | echinoderm microtubule associated protein like 2 |
| 1355 | trans-golgi network protein 2 |
| 1356 | translocation associated membrane protein 2 |
| 1357 | pleckstrin |
| 1358 | rTS beta protein |
| 1359 | ubiquitin specific protease 46 |
| 1360 | N-glycanase 1 |
| 1361 | protein phosphatase 1, catalytic subunit, beta isoform |
| 1362 | golgi associated, gamma adaptin ear containing, ARF binding protein 2 |
| 1363 | — |
| 1364 | zinc finger protein 451 |
| 1365 | ubiquitin-conjugating enzyme E2 variant 1 /// ubiquitin-conjugating enzyme E2 variant 1 |
| 1366 | galactosylceramidase (Krabbe disease) |
| 1367 | cullin 1 |
| 1368 | Ras association (RalGDS/AF-6) domain family 2 |
| 1369 | fructose-1,6-bisphosphatase 1 |
| 1370 | ADP-ribosylation factor guanine nucleotide-exchange factor 1(brefeldin A-inhibited) |
| 1371 | chromosome 10 open reading frame 6 |
| 1372 | transmembrane protein 1 |
| 1373 | heat shock 70 kDa protein 4 |
| 1374 | STE20-like kinase (yeast) |
| 1375 | N-sulfoglucosamine sulfohydrolase (sulfamidase) |
| 1376 | GRB2-associated binding protein 2 |
| 1377 | rabconnectin-3 |
| 1378 | 1-acylglycerol-3-phosphate O-acyltransferase 1 (lysophosphatidic acid acyltransferase, alpha) |
| 1379 | protein phosphatase 3 (formerly 2B), regulatory subunit B, 19 kDa, alpha isoform (calcineurin B, type I) |
| 1380 | zinc finger protein 198 |
| 1381 | T-cell, immune regulator 1, ATPase, H+ transporting, lysosomal V0 protein a isoform 3 |
| 1382 | KIAA0754 protein |
| 1383 | NAD kinase |
| 1384 | RAP2C, member of RAS oncogene family |
| 1385 | calmodulin 3 (phosphorylase kinase, delta) |
| 1386 | leucine rich repeat (in FLII) interacting protein 1 |
| 1387 | farnesyltransferase, CAAX box, alpha |
| 1388 | calcium/calmodulin-dependent protein kinase kinase 2, beta |
| 1389 | scaffold attachment factor B |
| 1390 | Alport syndrome, mental retardation, midface hypoplasia and elliptocytosis chromosomal region, gene 1 |
| 1391 | PRP39 pre-mRNA processing factor 39 homolog (yeast) |
| 1392 | dendritic cell protein |
| 1393 | complement component 1, q subcomponent, receptor 1 |
| 1394 | MAX protein 1 |
| 1395 | zinc finger protein 36, C3H type, homolog (mouse) |
| 1396 | synaptotagmin binding, cytoplasmic RNA interacting protein 1 |
| 1397 | kinesin family member 21B |
| 1398 | — |
| 1399 | HRAS-like suppressor |
| 1400 | suppressor of Ty 3 homolog (S. cerevisiae) |
| 1401 | Mix interactor |
| 1402 | FN5 protein |
| 1403 | DKFZP434H132 protein |
| 1404 | tRNA selenocysteine associated protein |
| 1405 | — |
| 1406 | transmembrane protein 30A |
| 1407 | interleukin 8 receptor, beta |
| 1408 | KIAA0528 gene product |
| 1409 | MADS box transcription enhancer factor 2, polypeptide C (myocyte enhancer factor 2C) |
| 1410 | hydroxyacyl-Coenzyme A dehydrogenase/3-ketoacyl-Coenzyme A thiolase/enoyl-Coenzyme A hydrase (trifunctional protein), |
| | alpha subunit |
| 1411 | PAX transcription activation domain interacting protein 1 like |
| 1412 | chromosome 6 open reading frame 106 |
| 1413 | prolylcarboxypeptidase (angiotensinase C) |
| 1414 | DnaJ (Hsp40) homolog, subfamily B, member 6 |
| 1415 | guanine nucleotide binding protein (G protein), beta 5 |
| 1416 | neuroblastoma RAS viral (v-ras) oncogene homolog |
| 1417 | chromosome 16 open reading frame 44 |
| 1418 | ubiquitin specific protease 36 |
| 1419 | ORF |
| 1420 | splicing factor, arginine/serine-rich 1 (splicing factor 2, alternate splicing factor) |
| 1421 | cytidine monophosphate N-acetylneuraminic acid synthetase |
| 1422 | interleukin 15 |
| 1423 | hypothetical protein FLJ11036 |
| 1424 | protein tyrosine phosphatase type IVA, member 1 |
| 1425 | protein-L-isoaspartate (D-aspartate) O-methyltransferase |
| 1426 | polymerase (DNA-directed), delta 3, accessory subunit |
| 1427 | activin A receptor, type I |
| 1428 | solute carrier family 1 (glutamate/neutral amino acid transporter), member 4 |
| 1429 | chromosome 10 open reading frame 86 |
| 1430 | CDC14 cell division cycle 14 homolog B (S. cerevisiae) |
| 1431 | phosphodiesterase 8A |
| 1432 | oxysterol binding protein-like 3 |
| 1433 | — |
| 1434 | tryptophan rich basic protein |
| 1435 | APG16 autophagy 16-like (S. cerevisiae) |
| 1436 | BCL2-associated athanogene |
| 1437 | protein tyrosine phosphatase-like (proline instead of catalytic arginine), member b |
| 1438 | CDC42 effector protein (Rho GTPase binding) 3 |
| 1439 | tubulin beta MGC4083 |
| 1440 | transmembrane 4 superfamily member 8 |
| 1441 | peptidyl arginine deiminase, type IV |
| 1442 | WAS protein family, member 2 |
| 1443 | — |
| 1444 | SRY (sex determining region Y)-box 15 |
| 1445 | isocitrate dehydrogenase 3 (NAD+) beta |
| 1446 | family with sequence similarity 49, member A |
| 1447 | PLSC domain containing protein |
| 1448 | family with sequence similarity 13, member A1 |
| 1449 | mitogen-activated protein kinase kinase kinase kinase 1 |
| 1450 | homogentisate 1,2-dioxygenase (homogentisate oxidase) |
| 1451 | heat shock 90 kDa protein 1, beta /// heat shock 90 kDa protein 1, beta |
| 1452 | Rho guanine nucleotide exchange factor (GEF) 7 |
| 1453 | butyrophilin, subfamily 3, member A2 |
| 1454 | KIAA1718 protein |
| 1455 | dCMP deaminase |
| 1456 | coated vesicle membrane protein |
| 1457 | ATPase, H+ transporting, lysosomal 34 kDa, V1 subunit D |
| 1458 | cyclin G2 |
| 1459 | adenosine monophosphate deaminase 2 (isoform L) |
| 1460 | sema domain, immunoglobulin domain (Ig), transmembrane domain (TM) and short cytoplasmic domain, (semaphorin) 4A |
| 1461 | SEC14-like 1 (S. cerevisiae) |
| 1462 | CD36 antigen (collagen type I receptor, thrombospondin receptor) |
| 1463 | kinesin family member 5C |
| 1464 | granulysin |
| 1465 | N-terminal asparagine amidase |
| 1466 | cisplatin resistance associated |
| 1467 | seven in absentia homolog 2 (Drosophila) |
| 1468 | splicing factor, arginine/serine-rich 1 (splicing factor 2, alternate splicing factor) |
| 1469 | BRCA1 associated RING domain 1 |
| 1470 | family with sequence similarity 34, member A |
| 1471 | YTH domain family 1 |
| 1472 | forkhead box K2 |
| 1473 | latent transforming growth factor beta binding protein 3 |
| 1474 | ribonuclease, RNase A family, 2 (liver, eosinophil-derived neurotoxin) |
| 1475 | — |
| 1476 | centaurin, delta 2 |
| 1477 | UDP-glucose pyrophosphorylase 2 |
| 1478 | family with sequence similarity 49, member A /// family with sequence similarity 49, member A |
| 1479 | C-type (calcium dependent, carbohydrate-recognition domain) lectin, superfamily member 6 |
| 1480 | coatomer protein complex, subunit gamma |
| 1481 | — |
| 1482 | interleukin 18 receptor accessory protein |
| 1483 | nuclear receptor coactivator 2 |
| 1484 | triple functional domain (PTPRF interacting) |
| 1485 | ring finger protein 24 |
| 1486 | hypothetical protein CG003 |
| 1487 | joined to JAZF1 |
| 1488 | TAR (HIV) RNA binding protein 1 |
| 1489 | phosphatidylinositol 4-kinase, catalytic, beta polypeptide |
| 1490 | triggering receptor expressed on myeloid cells 1 |
| 1491 | replication protein A4, 34 kDa |
| 1492 | UPF3 regulator of nonsense transcripts homolog A (yeast) |
| 1493 | MDN1, midasin homolog (yeast) |
| 1494 | RAB1A, member RAS oncogene family |
| 1495 | cytochrome P450, family 1, subfamily B, polypeptide 1 |
| 1496 | splicing factor, arginine/serine-rich 14 |
| 1497 | caspase recruitment domain family, member 15 |
| 1498 | component of oligomeric golgi complex 7 |
| 1499 | insulin induced gene 1 |
| 1500 | synovial sarcoma translocation, chromosome 18 |
| 1501 | platelet-activating factor acetylhydrolase, isoform Ib, alpha subunit 45 kDa |
| 1502 | parvin, beta |
| 1503 | hypermethylated in cancer 2 |
| 1504 | catalase /// catalase |
| 1505 | solute carrier family 31 (copper transporters), member 2 |
| 1506 | MCM3 minichromosome maintenance deficient 3 (S. cerevisiae) associated protein |
| 1507 | SWI/SNF related, matrix associated, actin dependent regulator of chromatin, subfamily a, member 4 |
| 1508 | sulfotransferase family, cytosolic, 1A, phenol-preferring, member 1 |
| 1509 | choline phosphotransferase 1 |
| 1510 | KIAA0999 protein |
| 1511 | ELISC-1 mRNA, partial cds |
| 1512 | — |
| 1513 | ubiquitin-conjugating enzyme E2G 1 (UBC7 homolog, C. elegans) |
| 1514 | SAM domain, SH3 domain and nuclear localisation signals, 1 |
| 1515 | DAZ associated protein 2 |
| 1516 | LIM protein (similar to rat protein kinase C-binding enigma) |
| 1517 | hydatidiform mole associated and imprinted |
| 1518 | hypothetical protein FLJ10178 |
| 1519 | guanine monphosphate synthetase |
| 1520 | heterogeneous nuclear ribonucleoprotein F |
| 1521 | RNA processing factor 1 |
| 1522 | elaC homolog 2 (E. coli) |
| 1523 | nuclear factor of kappa light polypeptide gene enhancer in B-cells 1 (p105) |
| 1524 | mitogen-activated protein kinase kinase 6 |
| 1525 | CD151 antigen |
| 1526 | protein kinase C binding protein 1 |
| 1527 | ankyrin repeat and MYND domain containing 2 |
| 1528 | SMC1 structural maintenance of chromosomes 1-like 1 (yeast) |
| 1529 | ras-related C3 botulinum toxin substrate 1 (rho family, small GTP binding protein Rac1) |
| 1530 | ferrochelatase (protoporphyria) |
| 1531 | transmembrane protein 1 |
| 1532 | v-ets erythroblastosis virus E26 oncogene homolog 2 (avian) |
| 1533 | trichorhinophalangeal syndrome I |
| 1534 | CDNA FLJ38765 fis, clone KIDNE2014489 |
| 1535 | CDNA FLJ14136 fis, clone MAMMA1002744 |
| 1536 | chemokine (C-C motif) receptor 6 |
| 1537 | CASP8 and FADD-like apoptosis regulator |
| 1538 | mitogen-activated protein kinase kinase 3 |
| 1539 | ADP-ribosylation factor guanine nucleotide-exchange factor 2 (brefeldin A-inhibited) |
| 1540 | protein phosphatase 2, regulatory subunit B (B56), gamma isoform |
| 1541 | v-maf musculoaponeurotic fibrosarcoma oncogene homolog G (avian) |
| 1542 | KIAA0063 gene product |
| 1543 | hypothetical protein HSPC132 |
| 1544 | CDK2-associated protein 1 |
| 1545 | H1 histone family, member 0 |
| 1546 | signal peptidase complex (18 kD) |
| 1547 | protein phosphatase 1, regulatory (inhibitor) subunit 12A |
| 1548 | epimorphin |
| 1549 | RAP2A, member of RAS oncogene family |
| 1550 | nucleosome assembly protein 1-like 1 |
| 1551 | centrosome protein Cep63 |
| 1552 | leukocyte immunoglobulin-like receptor, subfamily B (with TM and ITIM domains), member 1 |
| 1553 | amyloid beta (A4) precursor protein (protease nexin-II, Alzheimer disease) |
| 1554 | Wilms tumor 1 associated protein |
| 1555 | zinc finger protein 451 |
| 1556 | Fc fragment of IgG, high affinity Ia, receptor for (CD64) |
| 1557 | Notch homolog 2 (Drosophila) |
| 1558 | calsyntenin 1 |
| 1559 | casein kinase 1, gamma 3 |
| 1560 | pantothenate kinase 3 |
| 1561 | hypothetical protein FLJ21272 |
| 1562 | UDP-N-acetyl-alpha-D-galactosamine:polypeptide N-acetylgalactosaminyltransferase 1 (GalNAc-T1) |
| 1563 | alkylglycerone phosphate synthase |
| 1564 | stress-associated endoplasmic reticulum protein 1 |
| 1565 | ceroid-lipofuscinosis, neuronal 2, late infantile (Jansky-Bielschowsky disease) |
| 1566 | basic leucine zipper and W2 domains 1 |
| 1567 | zinc finger protein 313 |
| 1568 | CDNA FLJ12102 fis, clone HEMBB1002684 |
| 1569 | — |
| 1570 | phospholipid scramblase 1 |
| 1571 | transketolase (Wemicke-Korsakoff syndrome) |
| 1572 | cell cycle progression 1 |
| 1573 | CBF1 interacting corepressor |
| 1574 | histone 1, H2bd |
| 1575 | laminin, gamma 1 (formerly LAMB2) |
| 1576 | 6-phosphofructo-2-kinase/fructose-2,6-biphosphatase 3 |
| 1577 | cytoplasmic FMR1 interacting protein 1 |
| 1578 | stabilin 1 |
| 1579 | KIAA0692 protein |
| 1580 | caspase 1, apoptosis-related cysteine protease (interleukin 1, beta, convertase) |
| 1581 | H2A histone family, member V |
| 1582 | PRKC, apoptosis, WT1, regulator |
| 1583 | ELAV (embryonic lethal, abnormal vision, Drosophila)-like 1 (Hu antigen R) |
| 1584 | RAB4A, member RAS oncogene family |
| 1585 | homogentisate 1,2-dioxygenase (homogentisate oxidase) |
| 1586 | dishevelled associated activator of morphogenesis 1 |
| 1587 | zinc finger protein 37b (KOX 21) |
| 1588 | v-myb myeloblastosis viral oncogene homolog (avian)-like 1 |
| 1589 | hypothetical protein FLJ20618 |
| 1590 | annexin A5 |
| 1591 | mitogen-activated protein kinase kinase kinase kinase 1 |
| 1592 | leucine-rich repeats and death domain containing |
| 1593 | CD63 antigen (melanoma 1 antigen) |
| 1594 | paraoxonase 2 |
| 1595 | ATPase, Class V, type 10D |
| 1596 | leptin receptor /// leptin receptor gene-related protein |
| 1597 | ADP-ribosylation factor 3 /// ADP-ribosylation factor 3 |
| 1598 | — |
| 1599 | hypothetical protein FLJ10707 |
| 1600 | chromosome 14 open reading frame 154 |
| 1601 | KIAA0367 |
| 1602 | solute carrier family 33 (acetyl-CoA transporter), member 1 |
| 1603 | nuclear receptor coactivator 3 |
| 1604 | basic helix-loop-helix domain containing, class B, 2 |
| 1605 | AU RNA binding protein/enoyl-Coenzyme A hydratase |
| 1606 | ATPase, Class I, type 8B, member 4 |
| 1607 | dihydrolipoamide S-acetyltransferase (E2 component of pyruvate dehydrogenase complex) |
| 1608 | — |
| 1609 | natural killer-tumor recognition sequence |
| 1610 | PR/SET domain containing protein 8 |
| 1611 | male sterility domain containing 1 |
| 1612 | phosphodiesterase 4A, cAMP-specific (phosphodiesterase E2 dunce homolog, Drosophila) |
| 1613 | tensin /// tensin |
| 1614 | sorting nexin family member 27 |
| 1615 | kinesin heavy chain member 2 |
| 1616 | solute carrier organic anion transporter family, member 3A1 |
| 1617 | sialyltransferase 10 (alpha-2,3-sialyltransferase VI) |
| 1618 | hypothetical protein FLJ11822 |
| 1619 | zinc finger protein 258 |
| 1620 | — |
| 1621 | peptidyl arginine deiminase, type II |
| 1622 | UDP-glucuronate decarboxylase 1 |
| 1623 | sperm protein SSP411 |
| 1624 | trinucleotide repeat containing 15 |
| 1625 | cylindromatosis (turban tumor syndrome) |
| 1626 | GTP binding protein 4 |
| 1627 | microtubule-associated protein, RP/EB family, member 1 |
| 1628 | Kruppel-like factor 7 (ubiquitous) |
| 1629 | CD58 antigen, (lymphocyte function-associated antigen 3) |
| 1630 | histone 1, H2ad /// histone 1, H3d |
| 1631 | centaurin, beta 1 |
| 1632 | solute carrier family 16 (monocarboxylic acid transporters), member 5 |
| 1633 | hypothetical protein LOC339047 |
| 1634 | A kinase (PRKA) anchor protein 13 |
| 1635 | hypothetical protein FLJ20699 |
| 1636 | iduronate 2-sulfatase (Hunter syndrome) |
| 1637 | vacuolar protein sorting 39 (yeast) |
| 1638 | Tax1 (human T-cell leukemia virus type I) binding protein 1 |
| 1639 | cyclin-dependent kinase (CDC2-like) 11 |
| 1640 | solute carrier organic anion transporter family, member 3A1 |
| 1641 | thymidylate synthetase |
| 1642 | chromosome X open reading frame 45 |
| 1643 | |
| 1644 | APG5 autophagy 5-like (S. cerevisiae) |
| 1645 | formin-like 1 |
| 1646 | heparanase |
| 1647 | proteasome (prosome, macropain) 26S subunit, ATPase, 3 |
| 1648 | forkhead box O3A |
| 1649 | iduronate 2-sulfatase (Hunter syndrome) |
| 1650 | catenin (cadherin-associated protein), delta 1 |
| 1651 | RAB2, member RAS oncogene family |
| 1652 | cyclin D3 |
| 1653 | leptin receptor |
| 1654 | hypothetical protein FLJ10357 |
| 1655 | hypothetical protein FLJ12806 |
| 1656 | small membrane protein 1 |
| 1657 | oxysterol binding protein-like 8 |
| 1658 | transmembrane 9 superfamily protein member 4 |
| 1659 | SERTA domain containing 2 |
| 1660 | sorting nexin 6 |
| 1661 | interleukin 4 receptor |
| 1662 | tubulin, beta, 2 |
| 1663 | BTB and CNC homology 1, basic leucine zipper transcription factor 1 |
| 1664 | hypothetical protein FLJ12270 |
| 1665 | WD repeat and FYVE domain containing 3 |
| 1666 | EH-domain containing 1 |
| 1667 | solute carrier family 15 (H+/peptide transporter), member 2 |
| 1668 | endothelial differentiation, lysophosphatidic acid G-protein-coupled receptor, 4 |
| 1669 | phosphofructokinase, platelet |
| 1670 | ring finger protein 130 |
| 1671 | chromosome 22 open reading frame 5 |
| 1672 | OGT(O-Glc-NAc transferase)-interacting protein 106 KDa |
| 1673 | procollagen-proline, 2-oxoglutarate 4-dioxygenase (proline 4-hydroxylase), beta polypeptide (protein disulfide isomerase; thyroid |
| | hormone binding protein p55) |
| 1674 | ankyrin repeat domain 11 |
| 1675 | MADS box transcription enhancer factor 2, polypeptide C (myocyte enhancer factor 2C) |
| 1676 | hypothetical protein FLJ11301 |
| 1677 | BCL2-associated X protein |
| 1678 | Clone HA1 anti-HAV capsid immunoglobulin G heavy chain variable region mRNA, partial cds /// Clone HA1 anti-HAV capsid |
| | immunoglobulin G heavy chain variable region mRNA, partial cds |
| 1679 | lectin, galactoside-binding, soluble, 8 (galectin 8) |
| 1680 | splicing factor 3b, subunit 1, 155 kDa |
| 1681 | hypothetical protein FLJ12484 |
| 1682 | cysteine-rich motor neuron 1 |
| 1683 | hypothetical protein FLJ10613 |
| 1684 | nodal modulator 2 /// pM5 protein, middle copy |
| 1685 | proteasome (prosome, macropain) subunit, beta type, 1 |
| 1686 | sulfotransferase family, cytosolic, 1A, phenol-preferring, member 3 |
| 1687 | protein tyrosine phosphatase type IVA, member 3 |
| 1688 | Clone IMAGE: 5311129, mRNA |
| 1689 | hypothetical protein FLJ10948 |
| 1690 | LUC7-like 2 (S. cerevisiae) |
| 1691 | KIAA0922 protein |
| 1692 | zinc finger protein 395 |
| 1693 | coactosin-like 1 (Dictyostelium) |
| 1694 | phosphoserine phosphatase |
| 1695 | neuropathy target esterase |
| 1696 | WD repeat domain 6 |
| 1697 | zinc finger and BTB domain containing 16 |
| 1698 | interleukin enhancer binding factor 2, 45 kDa /// interleukin enhancer binding factor 2, 45 kDa |
| 1699 | 4-aminobutyrate aminotransferase |
| 1700 | chromosome 19 open reading frame 22 |
| 1701 | mitogen-activated protein kinase kinase kinase kinase 1 |
| 1702 | KIAA0863 protein |
| 1703 | dynactin 6 |
| 1704 | IGF-II mRNA-binding protein 2 |
| 1705 | KIAA0826 |
| 1706 | isocitrate dehydrogenase 3 (NAD+) alpha |
| 1707 | v-raf-1 murine leukemia viral oncogene homolog 1 |
| 1708 | ubiquitin associated domain containing 1 |
| 1709 | SH3 domain and tetratricopeptide repeats 1 |
| 1710 | microtubule-actin crosslinking factor 1 |
| 1711 | v-ral simian leukemia viral oncogene homolog B (ras related; GTP binding protein) |
| 1712 | heterogeneous nuclear ribonucleoprotein U (scaffold attachment factor A) |
| 1713 | hypothetical protein MGC2655 |
| 1714 | polymerase (DNA directed), epsilon 3 (p17 subunit) |
| 1715 | major histocompatibility complex, class II, DQ beta 1 /// major histocompatibility complex, class II, DQ beta 1 |
| 1716 | transmembrane 7 superfamily member 1 (upregulated in kindney) |
| 1717 | leucine-rich repeats and immunoglobulin-like domains 1 /// leucine-rich repeats and immunoglobulin-like domains 1 |
| 1718 | heart and neural crest derivatives expressed 2 |
| 1719 | cold autoinflammatory syndrome 1 |
| 1720 | sodium channel, nonvoltage-gated 1 alpha |
| 1721 | inositol 1,4,5-triphosphate receptor, type 1 |
| 1722 | nuclear receptor coactivator 1 |
| 1723 | proapoptotic caspase adaptor protein |
| 1724 | hypothetical gene CG030 |
| 1725 | phospholipase A2, group XIIA /// phospholipase A2, group XIIA |
| 1726 | RNA binding motif protein 25 |
| 1727 | leukocyte immunoglobulin-like receptor, sbufamily A (with TM domain), member 2 |
| 1728 | CTAGE family, member 5 |
| 1729 | — |
| 1730 | vitamin D (1,25-dihydroxyvitamin D3) receptor |
| 1731 | phosphatidylinositol-3-phosphate/phosphatidylinositol 5-kinase, type III |
| 1732 | haptoglobin |
| 1733 | CDNA FLJ14343 fis, clone THYRO1000916 |
| 1734 | sema domain, immunoglobulin domain (Ig), transmembrane domain (TM) and short cytoplasmic domain, (semaphorin) 4D |
| 1735 | inositol 1,4,5-triphosphate receptor, type 3 |
| 1736 | loss of heterozygosity, 11, chromosomal region 2, gene A |
| 1737 | POU domain, class 2, transcription factor 2 /// POU domain, class 2, transcription factor 2 |
| 1738 | meningioma expressed antigen 5 (hyaluronidase) |
| 1739 | tetratricopeptide repeat domain 3 |
| 1740 | MRNA; cDNA DKFZp566C093 (from clone DKFZp566C093) |
| 1741 | zyxin |
| 1742 | chaperonin containing TCP1, subunit 6B (zeta 2) |
| 1743 | chromosome 13 open reading frame 18 |
| 1744 | nasopharyngeal carcinoma associated gene protein-8 |
| 1745 | NEDD9 interacting protein with calponin homology and LIM domains |
| 1746 | phosphodiesterase 4A, cAMP-specific (phosphodiesterase E2 dunce homolog, Drosophila) |
| 1747 | apolipoprotein B mRNA editing enzyme, catalytic polypeptide-like 3A |
| 1748 | SMT3 suppressor of mif two 3 homolog 2 (yeast) |
| 1749 | v-yes-1 Yamaguchi sarcoma viral related oncogene homolog |
| 1750 | camitine palmitoyltransferase 1A (liver) |
| 1751 | paired immunoglobin-like type 2 receptor alpha |
| 1752 | ATPase, Na+/K+ transporting, beta 3 polypeptide |
| 1753 | formin binding protein 3 |
| 1754 | calpain 3, (p94) |
| 1755 | cyclin-dependent kinase inhibitor 1C (p57, Kip2) |
| 1756 | H3 histone, family 3B (H3.3B) |
| 1757 | DKFZp434A0131 protein |
| 1758 | CUG triplet repeat, RNA binding protein 1 |
| 1759 | metastasis suppressor 1 |
| 1760 | nucleoporin 50 kDa |
| 1761 | transducin-like enhancer of split 3 (E(sp1) homolog, Drosophila) |
| 1762 | RER1 homolog (S. cerevisiae) |
| 1763 | chromosome 1 open reading frame 38 |
| 1764 | glycoprotein IX (platelet) |
| 1765 | leukocyte-associated Ig-like receptor 1 |
| 1766 | dystonin |
| 1767 | CD164 antigen, sialomucin |
| 1768 | ligase IV, DNA, ATP-dependent |
| 1769 | TIA1 cytotoxic granule-associated RNA binding protein-like 1 |
| 1770 | neurofibromin 1 (neurofibromatosis, von Recklinghausen disease, Watson disease) |
| 1771 | v-yes-1 Yamaguchi sarcoma viral oncogene homolog 1 |
| 1772 | interferon-induced protein 44 |
| 1773 | immunoglobulin heavy constant mu |
| 1774 | death effector domain containing |
| 1775 | proteasome (prosome, macropain) subunit, alpha type, 1 /// proteasome (prosome, macropain) subunit, alpha type, 1 |
| 1776 | TSC-22-like /// TSC-22-like |
| 1177 | mediator of DNA damage checkpoint 1 |
| 1778 | solute carrier family 2 (facilitated glucose transporter), member 14 /// solute carrier family 2 (facilitated glucose transporter), |
| | member 3 |
| 1779 | heme-regulated initiation factor 2-alpha kinase |
| 1780 | heme binding protein 1 |
| 1781 | megalencephalic leukoencephalopathy with subcortical cysts 1 |
| 1782 | activated RNA polymerase II transcription cofactor 4 |
| 1783 | KIAA1052 protein |
| 1784 | cerebral cavemous malformations 1 |
| 1785 | protein phosphatase 6, catalytic subunit |
| 1786 | cyclin-dependent kinase 7 (MO15 homolog, Xenopus laevis, cdk-activating kinase) |
| 1787 | likely ortholog of C. elegans anterior pharynx defective 1A |
| 1788 | N-ethylmaleimide-sensitive factor attachment protein, alpha |
| 1789 | proteasome (prosome, macropain) 26S subunit, non-ATPase, 12 |
| 1790 | synaptophysin-like protein |
| 1791 | eukaryotic translation termination factor 1 |
| 1792 | c-mir, cellular modulator of immune recognition |
| 1793 | ARP2 actin-related protein 2 homolog (yeast) |
| 1794 | diaphanous homolog 1 (Drosophila) |
| 1795 | proprotein convertase subtilisin/kexin type 7 |
| 1796 | eukaryotic translation initiation factor 4 gamma, 3 |
| 1797 | jumonji domain containing 1B |
| 1798 | chromosome 13 open reading frame 23 |
| 1799 | WD repeat domain 8 |
| 1800 | myosin XVB, pseudogene |
| 1801 | protein tyrosine phosphatase, non-receptor type substrate 1 |
| 1802 | thioredoxin domain containing 7 (protein disulfide isomerase) |
| 1803 | myeloproliferative leukemia virus oncogene |
| 1804 | phosphatidylcholine transfer protein |
| 1805 | caspase 10, apoptosis-related cysteine protease |
| 1806 | macrophage receptor with collagenous structure |
| 1807 | human immunodeficiency virus type I enhancer binding protein 2 |
| 1808 | a disintegrin and metalloproteinase domain 8 |
| 1809 | — |
| 1810 | dual specificity phosphatase 12 |
| 1811 | Williams-Beuren syndrome chromosome region 5 |
| 1812 | nudix (nucleoside diphosphate linked moiety X)-type motif 1 |
| 1813 | NIMA (never in mitosis gene a)-related kinase 3 |
| 1814 | splicing factor, arginine/serine-rich 9 |
| 1815 | reticulon 4 |
| 1816 | makorin, ring finger protein, 1 |
| 1817 | serum response factor (c-fos serum response element-binding transcription factor) |
| 1818 | poly(A) polymerase alpha |
| 1819 | chromosome 12 open reading frame 4 |
| 1820 | hypothetical protein LOC339047 /// nuclear pore complex interacting protein |
| 1821 | zinc finger, DHHC domain containing 17 |
| 1822 | pyruvate dehydrogenase (lipoamide) alpha 1 |
| 1823 | dysferlin, limb gridle muscular dystrophy 2B (autosomal recessive) |
| 1824 | basic transcription factor 3 |
| 1825 | major histocompatibility complex, class II, DQ beta 1 |
| 1826 | centaurin, delta 2 |
| 1827 | tumor necrosis factor receptor superfamily, member 1B |
| 1828 | RNA binding motif protein 25 |
| 1829 | solute carrier family 18 (vesicular monoamine), member 2 |
| 1830 | integrin, alpha 4 (antigen CD49D, alpha 4 subunit of VLA-4 receptor) |
| 1831 | zinc finger, CCHC domain containing 14 |
| 1832 | zinc finger protein 36, C3H type-like 2 |
| 1833 | retinoid X receptor, alpha |
| 1834 | plexin D1 |
| 1835 | nuclear receptor coactivator 1 |
| 1836 | platelet-activating factor acetylhydrolase, isoform lb, alpha subunit 45 kDa |
| 1837 | peptidoglycan recognition protein 1 |
| 1838 | heat shock 27 kDa protein 1 |
| 1839 | core-binding factor, runt domain, alpha subunit 2; translocated to, 3 |
| 1840 | pre-mRNA cleavage complex II protein Pcf11 |
| 1841 | PR domain containing 10 |
| 1842 | aryl hydrocarbon receptor nuclear translocator-like |
| 1843 | lamin B2 |
| 1844 | ATP-binding cassette, sub family B (MDR/TAP), member 4 |
| 1845 | — |
| 1846 | RNA binding protein 3 |
| 1847 | CDK5 regulatory subunit associated protein 3 |
| 1848 | A kinase (PRKA) anchor protein 2 |
| 1849 | potassium channel modulatory factor 1 |
| 1850 | brain acyl-CoA hydrolase |
| 1851 | transcriptional regulator protein |
| 1852 | calpain, small subunit 1 /// calpain, small subunit 1 |
| 1853 | OTU domain, ubiquitin aldehyde binding 1 |
| 1854 | APG5 autophagy 5-like (S. cerevisiae) |
| 1855 | RAB11A, member RAS oncogene family |
| 1856 | gasdermin-like |
| 1857 | unactive progesterone receptor, 23 kD |
| 1858 | glycogenin |
| 1859 | karyopherin alpha 4 (importin alpha 3) |
| 1860 | WD repeat and SOCS box-containing 2 |
| 1861 | FK506 binding protein 11, 19 kDa |
| 1862 | heterogeneous nuclear ribonucleoprotein H1 (H) |
| 1863 | proteasome regulatory particle subunit p44S10 |
| 1864 | CDNA FLJ43274 fis, clone KIDNE2008914 |
| 1865 | serine (or cysteine) proteinase inhibitor, clade A (alpha-1 antiproteinase, antitrypsin), member 1 |
| 1866 | armadillo repeat containing, X-linked 3 |
| 1867 | CD86 antigen (CD28 antigen ligand 2, B7-2 antigen) |
| 1868 | parvin, beta |
| 1869 | iduronate 2-sulfatase (Hunter syndrome) |
| 1870 | lysosomal associated protein transmembrane 4 beta |
| 1871 | platelet derived growth factor C |
| 1872 | translokin |
| 1873 | TATA box binding protein (TBP)-associated factor, RNA polymerase I, C, 110 kDa |
| 1874 | sialic acid binding Ig-like lectin 8 |
| 1875 | RAS guanyl releasing protein 3 (calcium and DAG-regulated) |
| 1876 | ribosomal protein S6 kinase, 52 kDa, polypeptide 1 |
| 1877 | transcription factor 3 (E2A immunoglobulin enhancer binding factors E12/E47) |
| 1878 | 3-oxoacid CoA transferase 1 |
| 1879 | THO complex 1 |
| 1880 | peptidylprolyl isomerase B (cyclophilin B) |
| 1881 | KIAA0339 gene product |
| 1882 | likely ortholog of chicken chondrocyte protein with a poly-proline region |
| 1883 | ribonuclease, RNase A family, 4 |
| 1884 | amyloid beta (A4) precursor-like protein 2 |
| 1885 | transcriptional coactivator tubedown-100 |
| 1886 | KIAA0980 protein |
| 1887 | ubiquitin-conjugating enzyme E2D 3 (UBC4/5 homolog, yeast) |
| 1888 | insulin receptor substrate 2 |
| 1889 | tyrosine kinase 2 |
| 1890 | — |
| 1891 | MMS19-like (MET18 homolog, S. cerevisiae) |
| 1892 | reticulon 4 |
| 1893 | leucine-rich repeats and calponin homology (CH) domain containing 4 |
| 1894 | centaurin, delta 1 |
| 1895 | KIAA0251 protein |
| 1896 | immunoglobulin kappa constant /// Isolate M5 immunoglobulin kappa light chain variable region mRNA, partial cds /// Partial |
| | mRNA for immunoglobulin kappa chain variable region, (V1-Vk gene), from multiple myeloma: MM-8k /// Clone P2-32 anti- |
| | oxidized LDL immunoglobulin light chain Fab mRNA, partial cds /// Immunoglobulin kappa light chain VKJ region mRNA, partial |
| | cds |
| 1897 | aminopeptidase-like 1 |
| 1898 | tropomyosin 4 |
| 1899 | dihydropyrimidinase-like 2 |
| 1900 | von Willebrand factor |
| 1901 | mannosidase, alpha, class 1A, member 2 |
| 1902 | thioredoxin reductase 1 |
| 1903 | ariadne homolog, ubiquitin-conjugating enzyme E2 binding protein, 1 (Drosophila) |
| 1904 | tubulin tyrosine ligase-like family, member 3 |
| 1905 | cell division cycle 27 |
| 1906 | tyrosine 3-monooxygenase/tryptophan 5-monooxygenase activation protein, zeta polypeptide |
| 1907 | MAX protein |
| 1908 | sperm associated antigen 9 |
| 1909 | tumor necrosis factor receptor superfamily, member 6 |
| 1910 | MASK-4E-BP3 alternate reading frame gene |
| 1911 | amyotrophic lateral sclerosis 2 (juvenile) chromosome region, candidate 3 |
| 1912 | v-myb myeloblastosis viral oncogene homolog (avian) |
| 1913 | chaperonin containing TCP1, subunit 5 (epsilon) |
| 1914 | platelet-derived growth factor alpha polypeptide |
| 1915 | RNA binding motif protein 4 |
| 1916 | Fc fragment of IgG, receptor, transporter, alpha |
| 1917 | stromal membrane-associated protein 1 |
| 1918 | DDHD domain containing 2 |
| 1919 | 6-pyruvoyltetrahydropterin synthase |
| 1920 | karyopherin alpha 6 (importin alpha 7) |
| 1921 | SEC31-like 2 (S. cerevisiae) |
| 1922 | Vpr-binding protein |
| 1923 | ELOVL family member 5, elongation of long chain fatty acids (FEN1/Elo2, SUR4/Elo3-like, yeast) |
| 1924 | chromodomain helicase DNA binding protein 7 |
| 1925 | Clone 23705 mRNA sequence |
| 1926 | vacuolar protein sorting 45A (yeast) |
| 1927 | desmin /// family with sequence similarity 48, member A |
| 1928 | growth differentiation factor 2 |
| 1929 | syntaphilin |
| 1930 | anaphase promoting complex subunit 5 |
| 1931 | transducer of ERBB2, 1 |
| 1932 | tumor protein p53 binding protein, 2 |
| 1933 | hypothetical protein MGC3265 |
| 1934 | electron-transferring-flavoprotein dehydrogenase |
| 1935 | — |
| 1936 | CHMP1.5 protein |
| 1937 | heterogeneous nuclear ribonucleoprotein H3 (2H9) |
| 1938 | hypothetical protein FLJ22679 |
| 1939 | thyroid hormone receptor associated protein 1 |
| 1940 | cylindromatosis (turban tumor syndrome) |
| 1941 | discs, large homolog 4 (Drosophila) |
| 1942 | — |
| 1943 | neutrophil cytosolic factor 2 (65 kDa, chronic granulomatous disease, autosomal 2) |
| 1944 | purinergic receptor P2X, ligand-gated ion channel, 4 |
| 1945 | hypothetical protein FLJ10159 |
| 1946 | 5-methyltetrahydrofolate-homocysteine methyltransferase reductase |
| 1947 | chromosome 6 open reading frame 75 |
| 1948 | interleukin 1 receptor, type II |
| 1949 | iduronidase, alpha-L- |
| 1950 | chromodomain helicase DNA binding protein 1-like |
| 1951 | transducin (beta)-like 1X-linked |
| 1952 | hypothetical protein FLJ13725 |
| 1953 | FUS interacting protein (serine-arginine rich) 1 |
| 1954 | FLJ20259 protein |
| 1955 | BTG family, member 2 |
| 1956 | inhibitor of growth family, member 1-like |
| 1957 | chromosome 11 open reading frame 23 |
| 1958 | solute carrier family 36 (proton/amino acid symporter), member 1 |
| 1959 | mitogen-activated protein kinase 8 interacting protein 2 |
| 1960 | POU domain, class 2, transcription factor 1 |
| 1961 | Rho GDP dissociation inhibitor (GDI) alpha /// Rho GDP dissociation inhibitor (GDI) alpha |
| 1962 | ras homolog gene family, member A /// ras homolog gene family, member A |
| 1963 | RW1 protein |
| 1964 | solute carrier family 30 (zinc transporter), member 9 |
| 1965 | Patched homolog (Drosophila) |
| 1966 | cell division cycle 2-like 1 (PITSLRE proteins) |
| 1967 | MRNA; cDNA DKFZp762M127 (from clone DKFZp762M127) |
| 1968 | hydroxysteroid (17-beta) dehydrogenase 4 |
| 1969 | small inducible cytokine subfamily E, member 1 (endothelial monocyte-activating) |
| 1970 | thioredoxin domain containing 4 (endoplasmic reticulum) |
| 1971 | peptidylprolyl isomerase F (cyclophilin F) |
| 1972 | POU domain, class 6, transcription factor 1 |
| 1973 | mago-nashi homolog, proliferation-associated (Drosophila) |
| 1974 | cAMP responsive element binding protein 1 |
| 1975 | calcium channel, voltage-dependent, alpha 1I subunit |
| 1976 | CASP8 and FADD-like apoptosis regulator |
| 1977 | leucine aminopeptidase 3 |
| 1978 | calcium binding protein 3 /// calcium binding protein 5 |
| 1979 | carnitine acetyltransferase |
| 1980 | PHD finger protein 3 |
| 1981 | phosphonoformate immuno-associated protein 5 |
| 1982 | sodium channel, voltage-gated, type I, beta |
| 1983 | hypothetical protein FLJ20186 |
| 1984 | inhibitor of kappa light polypeptide gene enhancer in B-cells, kinase beta |
| 1985 | Chediak-Higashi syndrome 1 |
| 1986 | splicing factor, arginine/serine-rich 6 |
| 1987 | HBS1-like (S. cerevisiae) |
| 1988 | zinc finger protein 297B |
| 1989 | cancer susceptibility candidate 3 |
| 1990 | Williams Beuren syndrome chromosome region 22 |
| 1991 | adenosine deaminase, RNA-specific, B1 (RED1 homolog rat) |
| 1992 | RYK receptor-like tyrosine kinase |
| 1993 | mitochondrial carrier homolog 2 (C. elegans) |
| 1994 | KIAA0971 |
| 1995 | ankyrin repeat domain 10 |
| 1996 | SET domain, bifurcated 1 |
| 1997 | discs, large homolog 5 (Drosophila) |
| 1998 | LPS-responsive vesicle trafficking, beach and anchor containing |
| 1999 | KIAA0478 gene product |
| 2000 | asparagine-linked glycosylation 6 homolog (yeast, alpha-1,3-glucosyltransferase) |
| 2001 | zinc finger CCCH type, antiviral 1 |
| 2002 | MAP-kinase activating death domain |
| 2003 | G protein-coupled receptor 144 |
| 2004 | NADH dehydrogenase (ubiquinone) 1 alpha subcomplex, 6, 14 kDa |
| 2005 | WD repeat and SOCS box-containing 1 |
| 2006 | — |
| 2007 | CDNA FLJ36340 fis, clone THYMU2006468 |
| 2008 | phosphodiesterase 4D interacting protein (myomegalin) |
| 2009 | caspase 8, apoptosis-related cysteine protease |
| 2010 | small nuclear ribonucleoprotein polypeptide A' |
| 2011 | phosphatase and actin regulator 4 |
| 2012 | membrane component, chromosome 11, surface marker 1 |
| 2013 | activating transcription factor 4 (tax-responsive enhancer element B67) |
| 2014 | protein phosphatase 1A (formerly 2C), magnesium-dependent, alpha isoform |
| 2015 | — |
| 2016 | excision repair cross-complementing rodent rapair deficiency, complementation group 3 (xeroderma pigmentosum group B |
| | complementing) |
| 2017 | hypothetical protein LOC130074 |
| 2018 | mitogen-activated protein kinase kinase kinase 6 |
| 2019 | regulator of Fas-induced apoptosis |
| 2020 | EphA4 |
| 2021 | proline-rich nuclear receptor coactivator 1 |
| 2022 | tubulin, beta, 2 |
| 2023 | TANK-binding kinase 1 |
| 2024 | CD1D antigen, d polypeptide |
| 2025 | pericentriolar material 1 |
| 2026 | serine carboxypeptidase 1 |
| 2027 | RB1-inducible coiled-coil 1 |
| 2028 | leukocyte tyrosine kinase |
| 2029 | protein kinase, lysine deficient 1 |
| 2030 | PTK2 protein tyrosine kinase 2 |
| 2031 | immunoglobulin kappa variable 1D-13 |
| 2032 | Fc fragment of IgG, high affinity Ia, raceptor for (CD64) |
| 2033 | hypothetical protein FLJ21106 |
| 2034 | pumilio homolog 1 (Drosophila) |
| 2035 | CD44 antigen (homing function and Indian blood group system) |
| 2036 | protein phosphatase 3 (formerly 2B), regulatory subunit B, 19 kDa, alpha isoform (calcineurin B, type I) |
| 2037 | — |
| 2038 | myristoylated alanine-rich protein kinase C substrate |
| 2039 | phospholipase A2, group VII (platelet-activating factor acetylhydrolase, plasma) |
| 2040 | heterogeneous nuclear ribonucleoprotein M |
| 2041 | CDNA FLJ46076 fis, clone TESTI2002293 |
| 2042 | fragile X mental retardation, autosomal homolog 1 |
| 2043 | lysosomal-associated membrane protein 2 |
| 2044 | KIAA0792 gene product |
| 2045 | chromosome 6 open reading frame 32 |
| 2046 | sorcin |
| 2047 | Rho GTPase activating protein 4 |
| 2048 | t-complex 1 |
| 2049 | ferrochelatase (protoporphyria) |
| 2050 | — |
| 2051 | hepatocyte growth factor (hepapoietin A; scatter factor) |
| 2052 | solute carrier family 4 (anion exchanger), member 1, adaptor protein |
| 2053 | HIV-1 Tat interactive protein 2, 30 kDa |
| 2054 | chromosome 2 open reading frame 24 |
| 2055 | MAP-kinase activating death domain |
| 2056 | acidic (leucine-rich) nuclear phosphoprotein 32 family, member A |
| 2057 | caldesmon 1 |
| 2058 | AHA1, activator of heat shock 90 kDa protein ATPase homolog 2 (yeast) |
| 2059 | HLA-B associated transcript 3 |
| 2060 | PAP associated domain containing 1 |
| 2061 | transcriptional intermediary factor 1 |
| 2062 | cyclic AMP phosphoprotein, 19 kD |
| 2063 | CDNA clone IMAGE: 5286091, partial cds |
| 2064 | sulfotransferase family, cytosolic, 1A, phenol-preferring, member 1 |
| 2065 | CD163 antigen |
| 2066 | Rho GTPase activating protein 19 |
| 2067 | serine protease inhibitor, Kunitz type, 2 |
| 2068 | katanin p80 (WD repeat containing) subunit B 1 |
| 2069 | isopentenyl-diphosphate delta isomerase |
| 2070 | RAN binding protein 5 |
| 2071 | catenin (cadherin-associated protein), beta 1, 88 kDa |
| 2072 | tangerin |
| 2073 | colony stimulating factor 2 receptor, alpha, low-affinity (granulocyte-macrophage) |
| 2074 | tumor protein p53 inducible protein 3 |
| 2075 | ring finger protein 126 |
| 2076 | checkpoint with forkhead and ring finger domains |
| 2077 | voltage-dependent anion channel 2 /// voltage-dependent anion channel 2 |
| 2078 | protein kinase, DNA-activated, catalytic polypeptide |
| 2079 | kelch-like 18 (Drosophila) |
| 2080 | unc-51-like kinase 2 (C. elegans) |
| 2081 | interferon-related developmental regulator 1 |
| 2082 | KIAA0146 protein |
| 2083 | glycosyltransferase-like 1 |
| 2084 | GTP binding protein 2 |
| 2085 | microtubule-associated protein, RP/EB family, member 1 |
| 2086 | ATPase, H+ transporting, lysosomal accessory protein 2 |
| 2087 | cell division cycle 2-like 1 (PITSLRE proteins) |
| 2088 | thromboxane A2 receptor |
| 2089 | RAN, member RAS oncogene family |
| 2090 | BCL2-associated athanogene 4 |
| 2091 | glutamine-fructose-6-phosphate transaminase 1 |
| 2092 | zinc finger protein-like 1 |
| 2093 | host cell factor C2 |
| 2094 | small nuclear ribonucleoprotein polypeptide A′ |
| 2095 | ras responsive element binding protein 1 |
| 2096 | chromosome 12 open reading frame 5 |
| 2097 | Rho GTPase activating protein 12 |
| 2098 | NIMA (never in mitosis gene a)-related kinase 9 |
| 2099 | programmed cell death 2 |
| 2100 | major histocompatibility complex, class II, DO beta |
| 2101 | ubiquitin-conjugating enzyme E2A (RAD6 homolog) |
| 2102 | chromodomain helicase DNA binding protein 9 |
| 2103 | thioredoxin domain containing 7 (protein disulfide isomerase) |
| 2104 | PRP4 pre-mRNA processing factor 4 homolog B (yeast) /// PRP4 pre-mRNA processing factor 4 homolog B (yeast) |
| 2105 | polymerase (RNA) II (DNA directed) polypeptide E, 25 kDa |
| 2106 | similar to KIAA0592 protein |
| 2107 | frequently rearranged in advanced T-cell lymphomas 2 |
| 2108 | aminomethyltransferase (glycine cleavage system protein T) |
| 2109 | synaptojanin 2 |
| 2110 | hypothetical protein FLJ14107 |
| 2111 | SEC63-like (S. cerevisiae) |
| 2112 | GABA(A) receptor-associated protein like 1 |
| 2113 | dynactin 1 (p150, glued homolog, Drosophila) /// dynactin 1 (p150, glued homolog, Drosophila) |
| 2114 | splicing factor 3a, subunit 1, 120 kDa |
| 2115 | formin binding protein 2 |
| 2116 | CDC42 binding protein kinase alpha (DMPK-like) |
| 2117 | aryl hydrocarbon receptor |
| 2118 | CK2 interacting protein 1; HQ0024c protein |
| 2119 | cytoplasmic linker associated protein 2 |
| 2120 | multiple inositol polyphosphate histidine phosphatase, 1 |
| 2121 | adenylosuccinate synthase |
| 2122 | Fc fragment of IgE, high affinity I, receptor for; gamma polypeptide |
| 2123 | ferritin, heavy polypeptide 1 |
| 2124 | ubiquitin-activating enzyme E1-domain containing 1 |
| 2125 | KIAA0261 |
| 2126 | E1A binding protein p400 |
| 2127 | Dicer1, Dcr-1 homolog (Drosophila) |
| 2128 | zinc finger, CCHC domain containing 2 |
| 2129 | KIAA1797 |
| 2130 | coronin 7 |
| 2131 | guanine nucleotide binding protein (G protein), beta 5 |
| 2132 | ATP-binding cassette, sub-family A (ABC1), member 1 |
| 2133 | potassium intermediate/small conductance calcium-activated channel, subfamily N, member 4 |
| 2134 | OGT(O-Glc-NAc transferase)-interacting protein 106 KDa |
| 2135 | ubiquitin-conjugating enzyme E2D 3 (UBC4/5 homolog, yeast) |
| 2136 | cyclin D2 |
| 2137 | retinoic acid receptor, alpha |
| 2138 | hypothetical protein FLJ10199 |
| 2139 | kynureninase (L-kynurenine hydrolase) |
| 2140 | solute carrier family 30 (zinc transporter), member 5 |
| 2141 | TGFB-induced factor (TALE family homeobox) |
| 2142 | copine III |
| 2143 | microtubule-actin crosslinking factor 1 |
| 2144 | I-mfa domain-containing protein /// I-mfa domain-containing protein |
| 2145 | APG7 autophagy 7-like (S. cerevisiae) |
| 2146 | hypothetical protein FLJ22054 |
| 2147 | — |
| 2148 | rabconnectin-3 |
| 2149 | cleavage stimulation factor, 3′ pre-RNA, subunit 2, 64 kDa, tau variant |
| 2150 | guanine nucleotide binding protein (G protein), beta polypeptide 2 |
| 2151 | programmed cell death 4 (neoplastic transformation inhibitor) |
| 2152 | bromodomain containing 3 |
| 2153 | stomatin |
| 2154 | ubiquitin-conjugating enzyme E2N (UBC13 homolog, yeast) |
| 2155 | gasdermin-like |
| 2156 | UDP-N-acetyl-alpha-D-galactosamine:polypeptide N-acetylgalactosaminyltransferase 3 (GalNAc-T3) |
| 2157 | Fc fragment of IgG, low affinity IIa, receptor for (CD32) /// Fc fragment of IgG, low affinity IIb, receptor for (CD32) /// Fc fragment |
| | of IgG, low affinity IIc, receptor for (CD32) |
| 2158 | gelsolin (amyloidosis, Finnish type) |
| 2159 | transducin-like enhancer of split 4 (E(sp1) homolog, Drosophila) |
| 2160 | IK cytokine, down-regulator of HLA II /// IK cytokine, down-regulator of HLA II |
| 2161 | CGI-51 protein |
| 2162 | poly(rC) binding protein 1 |
| 2163 | neural precursor cell expressed, developmentally down-regulated 5 |
| 2164 | WW domain containing E3 ubiquitin protein ligase 1 |
| 2165 | proteasome (prosome, macropain) subunit, alpha type, 1 |
| 2166 | guanine nucleotide binding protein (G protein), beta polypeptide 1 |
| 2167 | LOC388434 |
| 2168 | eukaryotic translation initiation factor 4E binding protein 3 |
| 2169 | lamin A/C |
| 2170 | protease, serine, 21 (testisin) |
| 2171 | ecotropic viral integration site 2B /// ecotropic viral integration site 2B |
| 2172 | lamin B1 |
| 2173 | dedicator of cytokinesis 9 |
| 2174 | calumenin |
| 2175 | — |
| 2176 | zinc finger, CCHC domain containing 14 |
| 2177 | chemokine-like factor super family 6 |
| 2178 | — |
| 2179 | heterogeneous nuclear ribonucleoprotein K /// heterogeneous nuclear ribonucleoprotein K |
| 2180 | H3 histone, family 3B (H3.3B) |
| 2181 | spastic paraplegia 7, paraplegin (pure and complicated autosomal recessive) |
| 2182 | hypothetical protein FLJ10824 /// similar to KIAA0592 protein |
| 2183 | v-kit Hardy-Zuckerman 4 feline sarcoma viral oncogene homolog |
| 2184 | integrin, alpha L (antigen CD11A (p180), lymphocyte function-associated antigen 1; alpha polypeptide) |
| 2185 | MCM3 minichromosome maintenance deficient 3 (S. cerevisiae) |
| 2186 | RAB40B, member RAS oncogene family |
| 2187 | chaperonin containing TCP1, subunit 2 (beta) |
| 2188 | citrate synthase |
| 2189 | KIAA0746 protein |
| 2190 | adiponectin receptor 2 |
| 2191 | calcium binding atopy-related autoantigen 1 |
| 2192 | eukaryotic translation initiation factor 2C, 4 |
| 2193 | acyl-CoA synthetase long-chain family member 4 |
| 2194 | spleen focus forming virus (SFFV) proviral integration oncogene spi1 |
| 2195 | death-associated protein kinase 3 |
| 2196 | PLSC domain containing protein |
| 2197 | four and a half LIM domains 1 |
| 2198 | transcription factor CP2 |
| 2199 | IQ motif containing E |
| 2200 | hypothetical protein FLJ12443 |
| 2201 | ATPase, H+ transporting, lysosomal accessory protein 1 |
| 2202 | cullin 2 |
| 2203 | hypothetical protein FLJ10824 |
| 2204 | proteasome (prosome, macropain) 26S subunit, non-ATPase, 8 |
| 2205 | SWI/SNF related, matrix associated, actin dependent regulator of chromatin, subfamily a, member 5 |
| 2206 | syndecan 4 (amphiglycan, ryudocan) |
| 2207 | paraneoplastic antigen MA1 |
| 2208 | fer-1-like 3, myoferlin (C. elegans) |
| 2209 | transgelin 2 |
| 2210 | related RAS viral (r-ras) oncogene homolog 2 |
| 2211 | BTB (POZ) domain containing 1 |
| 2212 | (hypothetical protein LOC339047 /// nuclear pore complex interacting protein |
| 2213 | cullin 4B |
| 2214 | NACHT, leucine rich repeat and PYD containing 1 |
| 2215 | mitochondrial isofeucine tRNA synthetase |
| 2216 | intersectin 1 (SH3 domain protein) |
| 2217 | solute carrier family 39 (zinc transporter), member 7 |
| 2218 | restin (Reed-Steinberg cell-expressed intermediate filament-associated protein) |
| 2219 | isoprenylcysteine carboxyl methyltransferase |
| 2220 | bone morphogenetic protein 6 |
| 2221 | rap2 interacting protein x |
| 2222 | cylindromatosis (turban tumor syndrome) |
| 2223 | surfactant, pulmonary-associated protein C |
| 2224 | leukocyte immunoglobulin-like receptor subfamily B (with TM and ITIM domains), member 2 |
| 2225 | CUG triplet repeat, RNA binding protein 2 |
| 2226 | polycystic kidney disease 2 (autosomal dominant) |
| 2227 | BCL2-associated transcription factor 1 |
| 2228 | solute carrier family 20 (phosphate transporter), member 1 |
| 2229 | tumor necrosis factor (ligand) superfamily, member 10 |
| 2230 | talin 1 |
| 2231 | oxidase (cytochrome c) assembly 1-like |
| 2232 | O-acetyltransferase |
| 2233 | PC4 and SFRS1 interacting protein 1 |
| 2234 | plasminogen activator, urokinase receptor |
| 2235 | cleavage and polyadenylation specific factor 5, 25 kDa |
| 2236 | splicing factor, arginine/serine-rich 8 (suppressor-of-white-apricot homolog, Drosophila) |
| 2237 | glycerol kinase |
| 2238 | vesicle-associated membrane protein 3 (cellubrevin) |
| 2239 | leptin receptor |
| 2240 | glucose phosphate isomerase |
| 2241 | APG5 autophagy 5-like (S. cerevisiae) |
| 2242 | diacylglycerol O-acyltransferase homolog 1 (mouse) |
| 2243 | casein kinase 2, alpha 1 polypeptide |
| 2244 | pleckstrin homology domain containing, family A (phosphoinositide binding specific) member 1 |
| 2245 | ribophorin I |
| 2246 | FLJ46603 protein |
| 2247 | Trophoblast-derived noncoding RNA |
| 2248 | SMAD, mothers against DPP homolog 3 (Drosophila) |
| 2249 | RAB, member of RAS oncogene family-like 2A /// RAB, member of RAS oncogene family-like 2B |
| 2250 | F-box protein 34 |
| 2251 | G protein-coupled receptor 21 |
| 2252 | laminin, alpha 2 (merosin, congenital muscular dystrophy) |
| 2253 | Clone 24629 mRNA sequence |
| 2254 | — |
| 2255 | dystonin |
| 2256 | hypothetical protein FLJ13910 |
| 2257 | MAP kinase interacting serine/threonine kinase 1 |
| 2258 | melanoma associated antigen (mutated) 1 |
| 2259 | tumor necrosis factor receptor superfamily, member 6 |
| 2260 | apoptosis-related protein PNAS-1 |
| 2261 | pleiomorphic adenoma gene-like 1 |
| 2262 | Ral GEF with PH domain and SH3 binding motif 1 |
| 2263 | phosphorylase kinase, alpha 2 (liver) |
| 2264 | mannosidase, alpha, class 2B, member 2 |
| 2265 | chromosome 14 open reading frame 11 |
| 2266 | glutamate dehydrogenase 1 |
| 2267 | butyrophilin, subfamily 3, member A2 |
| 2268 | LIM domain containing preferred translocation partner in lipoma |
| 2269 | parvin, beta |
| 2270 | N-acylsphingosine amidohydrolase (acid ceramidase) 1 |
| 2271 | Wolf-Hirschhom syndrome candidate 2 |
| 2272 | KIAA0685 |
| 2273 | BM-011 protein |
| 2274 | telomeric repeat binding factor 2, interacting protein |
| 2275 | — |
| 2276 | Endothelial-derived gene 1 |
| 2277 | fragile X mental retardation 1 |
| 2278 | ribonuclease P 14 kDa subunit |
| 2279 | calmodulin-like 4 |
| 2280 | signal transducer and activator of transcription 3 (acute-phase response factor) |
| 2281 | mitogen-activated protein kinase kinase kinase kinase 5 |
| 2282 | inositol 1,4,5-trisphosphate 3-kinase B |
| 2283 | oxysterol binding protein-like 2 |
| 2284 | intercellular adhesion molecule 4, Landsteiner-Wiener blood group |
| 2285 | CGI-72 protein |
| 2286 | phosphatase and tensin homolog (mutated in multiple advanced cancers 1) |
| 2287 | RNA (guanine-7-) methyltransferase |
| 2288 | interleukin 1 receptor antagonist |
| 2289 | flavin containing monooxygenase 5 |
| 2290 | RYK receptor-like tyrosine kinase |
| 2291 | destrin (actin depolymerizing factor) |
| 2292 | hypothetical protein FLJ12681 |
| 2293 | CGI-51 protein |
| 2294 | — |
| 2295 | chromosome 11 open reacting frame 21 |
| 2296 | catenin (cadherin-associated protein), alpha 1, 102 kDa |
| 2297 | mutS homolog 5 (E. coli) |
| 2298 | major histocompatibility complex, class I, B |
| 2299 | cyclin J |
| 2300 | core promoter element binding protein |
| 2301 | histone 1, H2bi |
| 2302 | B-cell CLL/lymphoma 7B |
| 2303 | nucleoporin 210 |
| 2304 | MRNA; cDNA DKFZp564G222 (from clone DKFZp564G222) |
| 2305 | succinate dehydrogenasa complex, subunit B, iron sulfur (lp) |
| 2306 | actinin, alpha 1 |
| 2307 | ring finger and CHY zinc finger domain containing 1 |
| 2308 | solute carrier family 12 (potassium/chloride transporters), member 6 |
| 2309 | regulator of G-protein signalling 2, 24 kDa |
| 2310 | tropomyosin 2 (beta) |
| 2311 | — |
| 2312 | UDP-N-acetyl-alpha-D-galactosamine:polypeptide N-acetylgalactosaminyltransferase 2 (GalNAc-T2) |
| 2313 | solute carrier family 22 (organic cation transporter), member 4 |
| 2314 | tyrosine 3-monooxygenase/tryptophan 5-monooxygenase activation protein, zeta polypeptide |
| 2315 | — |
| 2316 | septin 8 |
| 2317 | a disintegrin and metalloproteinase domain 23 |
| 2318 | ribonuclease H1 |
| 2319 | MYST histone acetyltransferase (monocytic leukemia) 3 |
| 2320 | Rho GTPase activating protein 26 |
| 2321 | BCL2-like 2 |
| 2322 | thyroid receptor interacting protein 15 |
| 2323 | nucleoporin 210 |
| 2324 | heat shock 70 kDa protein 9B (mortalin-2) |
| 2325 | kelch domain containing 2 |
| 2326 | KIAA0650 protein |
| 2327 | oxytocin receptor |
| 2328 | for protein disulfide isomerase-related |
| 2329 | scavenger receptor class B, member 2 |
| 2330 | LOC388969 |
| 2331 | CUG triplet repeat, RNA binding protein 1 |
| 2332 | diacylglycerol kinase, delta 130 kDa |
| 2333 | ribosomal protein S6 kinase, 90 kDa, polypeptide 2 |
| 2334 | T cell receptor delta diversity 3 /// T cell receptor delta locus |
| 2335 | v-rel reticuloendotheliosis viral oncogene homolog A, nuclear factor of kappa light polypeptide gene enhancer in B-cells 3, p65 |
| | (avian) |
| 2336 | phosphoribosylaminoimidazole carboxylase, phosphoribosylaminoimidazole succinocarboxamide synthetase |
| 2337 | HSPC063 protein |
| 2338 | phosphatidylinositol transfer protein, alpha |
| 2339 | MAX binding protein |
| 2340 | PRP18 pre-mRNA processing factor 18 homolog (yeast) |
| 2341 | fibrinogen-like 2 |
| 2342 | LIM domain only 2 (rhombotin-like 1) |
| 2343 | B-cell CLL/lymphoma 2 |
| 2344 | Ewing sarcoma breakpoint ragion 1 |
| 2345 | — |
| 2346 | serine hydroxymethyltransferase 2 (mitochondrial) |
| 2347 | ariadne homolog, ubiquitin-conjugating enzyme E2 binding protein, 1 (Drosophila) |
| 2348 | mutL homolog 3 (E. coli) |
| 2349 | guanine nucleotide binding protein (G protein), gamma 11 |
| 2350 | likely ortholog of mouse la related protein |
| 2351 | CDKN1A interacting zinc finger protein 1 |
| 2352 | E1A binding protein p300 |
| 2353 | LUC7-like (S. cerevisiae) |
| 2354 | ubiquitin-like 3 |
| 2355 | nuclear distribution gene C homolog (A. nidulans) |
| 2356 | MRNA; cDNA DKFZp762M127 (from clone DKFZp762M127) |
| 2357 | Ral GEF with PH domain and SH3 binding motif 2 |
| 2358 | protein tyrosine phosphatase type IVA, member 1 |
| 2359 | chemokine(C-C motif) receptor 3 |
| 2360 | immunoglobulin kappa constant |
| 2361 | KIAA0830 protein |
| 2362 | glucosidase, alpha; acid (Pompe disease, glycogen storage disease type II) |
| 2363 | hypothetical protein FLJ12681 |
| 2364 | — |
| 2365 | Janus kinase 2 (a protein tyrosine kinase) |
| 2366 | eukaryotic translation initiation factor 2-alpha kinase 3 |
| 2367 | UDP-N-acteylglucosamine pyrophosphorylase 1 |
| 2368 | IQ motif containing GTPase activating protein 2 |
| 2369 | lipin 1 |
| 2370 | metaxin 2 |
| 2371 | MRNA; cDNA DKFZp586K1123 (from clone DKFZp586K1123) |
| 2372 | emopamil binding protein (sterol isomerase) |
| 2373 | solute carrier family 35 (UDP-glucuronic acid/UDP-N-acetylgalactosamine dual transporter), member D1 |
| 2374 | RAD17 homolog (S. pombe) |
| 2375 | ATP/GTP binding protein 1 |
| 2376 | — |
| 2377 | low density lipoprotein receptor-related protein 10 |
| 2378 | high density lipoprotein binding protein (vigilin) |
| 2379 | DNA segment on chromosome 4 (unique) 234 expressed sequence |
| 2380 | PI-3-kinase-related kinase SMG-1-like |
| 2381 | valosin-containing protein (p97)/p47 complex-interacting protein p135 |
| 2382 | KIAA1466 protein |
| 2383 | karyopherin alpha 6 (importin alpha 7) |
| 2384 | Wiskott-Aldrich, syndrome protein interacting protein |
| 2385 | G protein-coupled receptor kinase 6 |
| 2386 | protein O-fucosyltransferase 1 |
| 2387 | serum amyloid A1 /// serum amyloid A2 /// serum amyloid A1 /// setum amyloid A2 |
| 2388 | HBS1-like (S. cerevisiae) |
| 2389 | — |
| 2390 | karyopherin (importin) beta 1 |
| 2391 | membrane-type 1 matrix metalloproteinase cytoplasmic tail binding protein-1 |
| 2392 | hypothetical protein FLJ11000 |
| 2393 | tumor-associated calcium signal transducer 2 |
| 2394 | widely-interspaced zinc finger motifs |
| 2395 | immunoglobulin superfamily, member 4 |
| 2396 | transducin-like enhancer of split 1 (E(sp1) homolog, Drosophila) |
| 2397 | hepatocyte growth factor-regulated tyrosine kinase substrate |
| 2398 | putative MAPK activating protein PM20, PM21 |
| 2399 | KIAA0420 gene product |
| 2400 | — |
| 2401 | splicing factor, arginine/serine-rich 10 (transformer 2 homolog, Drosophila) |
| 2402 | retinoblastoma binding protein 8 |
| 2403 | hypothetical protein FLJ20718 |
| 2404 | TRIAD3 protein |
| 2405 | — |
| 2406 | inositol polyphosphate-5-phosphatase, 40 kDa |
| 2407 | prostatic binding protein |
| 2408 | Chromobox homolog 5 (HP1 alpha homolog, Drosophila) |
| 2409 | hypothetical protein DT1P1A10 |
| 2410 | — |
| 2411 | immunoglobulin lambda joining 3 |
| 2412 | immunoglobulin kappa constant |
| 2413 | hexokinase 3 (white cell) |
| 2414 | KIAA0459 protein |
| 2415 | serine/threonine kinase 3 (STE20 homolog, yeast) |
| 2416 | cell division cycle 25A |
| 2417 | katanin p60 (ATPase-containing) subunit A 1 |
| 2418 | B-factor, properdin /// B-factor, properdin |
| 2419 | B lymphoid tyrosine kinase |
| 2420 | actin related protein 2/3 complex, subunit 2, 34 kDa |
| 2421 | additional sex combs like 1 (Drosophila) |
| 2422 | orosomucoid 2 |
| 2423 | ubiquitin-conjugating enzyme E2D 1 (UBC4/5 homolog, yeast) |
| 2424 | hexosaminidase B (beta polypeptide) |
| 2425 | nucleosome assembly protein 1-like 1 |
| 2426 | thyroid hormone receptor associated protein 5 |
| 2427 | carcinoembryonic antigen-related cell adhesion molecule 6 (non-specific cross reacting antigen) |
| 2428 | N-myristoyltransferase 1 |
| 2429 | SMAD, mothers against DPP homolog 2 (Drosophila) |
| 2430 | propionyl Coenzyme A carboxylase, beta polypeptide |
| 2431 | hypothetical protein FLJ21820 |
| 2432 | protein kinase C, nu |
| 2433 | E2F transcription factor 5, p130-binding |
| 2434 | protein tyrosine phosphatase type IVA, member 1 |
| 2435 | S-adenosylhomocysteine hydrolase |
| 2436 | neutrophil cytosolic factor 4, 40 kDa |
| 2437 | A kinase (PRKA) anchor protein 10 |
| 2438 | — |
| 2439 | Fc fragment of IgG, low affinity IIa, receptor for (CD32) /// Fc fragment of IgG, low affinity lib, receptor for (CD32) /// Fc fragment |
| | of IgG, low affinity IIc, receptor for (CD32) |
| 2440 | sperm associated antigen 8 |
| 2441 | hypothetical protein FLJ11259 |
| 2442 | likely homolog of rat kinase D-interacting substance of 220 kDa |
| 2443 | TBC1 domain family, member 15 |
| 2444 | HOM-TES-103 tumor antigen-like |
| 2445 | MRNA; cDNA DKFZp762M127 (from clone DKFZp762M127) |
| 2446 | CDC-like kinase 3 |
| 2447 | flavin containing monooxygenase 5 |
| 2448 | AT rich interactive domain 4A (RBP1-like) |
| 2449 | toll-like receptor 7 |
| 2450 | N-myc downstream regulated gene 1 |
| 2451 | splicing factor, arginine/serine-rich 6 |
| 2452 | forkhead box O1A (rhabdomyosarcoma) |
| 2453 | Rho guanine nucleotide exchange factor (GEF) 12 |
| 2454 | Chromosome 21q22.1 anonymous mRNA sequence |
| 2455 | zinc finger protein 313 |
| 2456 | protein kinase C, iota |
| 2457 | SAM domain and HD domain 1 |
| 2458 | mitochondrial translational release factor 1 |
| 2459 | homer homolog 2 (Drosophila) |
| 2460 | component of oligomeric golgi complex 8 /// peptide deformylase-like protein |
| 2461 | KIAA0258 |
| 2462 | transcription factor 3 (E2A immunoglobulin enhancer binding factors E12/E47) |
| 2463 | ATPase, Ca++ transporting, cardiac muscle, slow twitch 2 |
| 2464 | hypothetical protein FLJ23053 |
| 2465 | transcription factor 8 (represses interleukin 2 expression) |
| 2466 | pro-platelet basic protein (chemokine (C—X—C motif) ligand 7) |
| 2467 | ATP synthase, H+ transporting, mitochondrial F0 complex, subunit s (factor B) |
| 2468 | poly(rC) binding protein 4 |
| 2469 | calmin (calponin-like, transmembrane) |
| 2470 | chromosome 14 open reading frame 58 |
| 2471 | CGI-116 protein |
| 2472 | protein phosphatase 1G (formerly 2C), magnesium-dependent, gamma isoform |
| 2473 | synaptotagmin XI |
| 2474 | oxysterol binding protein-like 3 |
| 2475 | plasma glutamate carboxypeptidase |
| 2476 | son of sevenless homolog 1 (Drosophila) |
| 2477 | chromosome 10 open reading frame 57 |
| 2478 | ankyrin repeat domain 25 |
| 2479 | paired immunoglobin-like type 2 receptor beta |
| 2480 | succinate dehydrogenase complex, subunit C, integral membrane protein, 15 kDa |
| 2481 | dimethylarginine dimethylaminohydrolase 2 |
| 2482 | ATPase, Class VI, type 11A |
| 2483 | BCL2/adenovirus E18 19 kDa interacting protein 3-like |
| 2484 | CD44 antigen (homing function and Indian blood group system) |
| 2485 | ring finger protein 38 |
| 2486 | myeloid/lymphoid or mixed-lineage leukemia (trithorax homolog, Drosophila); translocated to, 2 |
| 2487 | UDP-glucose ceramide glucosyltransferase |
| 2488 | tumor necrosis factor receptor superfamily, member 12A |
| 2489 | myotubularin related protein 1 |
| 2490 | KIAA0217 |
| 2491 | immunoglobulin heavy constant gamma 1 (G1m marker) |
| 2492 | — |
| 2493 | destrin-2 pseudogene |
| 2494 | Kruppel-like factor 4 (gut) |
| 2495 | translocase of inner mitochondrial membrane 23 homolog (yeast) |
| 2496 | acetyl-Coenzyme A acyltransferase 2 (mitochondrial 3-oxoacyl-Coenzyme A thiolase) |
| 2497 | carboxypeptidase, vitellogenic-like /// carboxypeptidase, vitellogenic-like |
| 2498 | structure specific recognition protein 1 |
| 2499 | solute carrier family 25 (mitochondrial carrier, phosphate carrier), member 3 /// 3 solute carrier family 25 (mitochondrial carrier, |
| | phosphate carrier), member 3 |
| 2500 | — |
| 2501 | chromosome 14 open reading frame 94 |
| 2502 | hypothetical protein FLJ12205 /// phosducin-like 3 |
| 2503 | chromosome 16 open reading frame 7 |
| 2504 | restin (Reed-Steinberg cell-expressed intermediate filament-associated protein) |
| 2505 | transmembrane 4 superfamily member 13 |
| 2506 | acyl-CoA synthetase long-chain family member 1 |
| 2507 | phosphatidylinositol-3-phosphate associated protein |
| 2508 | hypothetical protein FLJ13910 |
| 2509 | dual-specificity tyrosine-(Y)-phosphorylation regulated kinase 2 |
| 2510 | C-terminal binding protein 2 |
| 2511 | HLA-B associated transcript 3 |
| 2512 | interferon regulatory factor 1 |
| 2513 | nischarin |
| 2514 | interleukin 1 receptor, type I |
| 2515 | A kinase (PRKA) anchor protein (yotiao) 9 |
| 2516 | Jumonji, AT rich interactive domain 1B (RBP2-like) |
| 2517 | coatomer protein complex, subunit alpha |
| 2518 | myeloid cell leukemia sequence 1 (BCL2-related) |
| 2519 | carnitine palmitoyltransferase 1B (muscle) |
| 2520 | — |
| 2521 | neural precursor cell expressed, developmentally down-regulated 5 /// neural precursor cell expressed, developmentally down- |
| | regulated 5 |
| 2522 | nuclear receptor coactivator 6 |
| 2523 | recombining binding protein suppressor of hairless (Drosophila) |
| 2524 | zinc finger protein 14 (KOX 6) |
| 2525 | epidermal growth factor receptor pathway substrate 15 |
| 2526 | cytoskeleton associated protein 1 |
| 2527 | prostaglandin E receptor 2 (subtype EP2), 53 kDa |
| 2528 | nuclear factor of activated T-cells, cytoplasmic, calcineurin-dependent 3 |
| 2529 | general transcription factor IIIC, polypeptide 1, alpha 220 kDa |
| 2530 | testis enhanced gene transcript (BAX inhibitor 1) |
| 2531 | integrin, alpha X (antigen CD11C (p150), alpha polypeptide) |
| 2532 | collagen, type VIII, alpha 2 |
| 2533 | ROD1 regulator of differentiation 1 (S. pombe) |
| 2534 | transketolase (Wernicke-Korsakoff syndrome) |
| 2535 | protein tyrosine phosphatase, receptor type, E |
| 2536 | KIAA0553 protein |
| 2537 | sulfotransferase family, cytosolic, 1A, phenol-preferring, member 2 |
| 2538 | ubiquitin specific protease 24 |
| 2539 | PHD finger protein 2 |
| 2540 | chromosome 20 open reading frame 36 |
| 2541 | chromosome 6 open reading frame 111 |
| 2542 | golgi associated, gamma adaptin ear containing, ARF binding protein 2 |
| 2543 | ankyrin repeat domain 15 |
| 2544 | chromosome 22 open reading frame 8 |
| 2545 | KIAA0792 gene product |
| 2546 | Sjogren syndrome antigen A2 (60 kDa, ribonucleoprotein autoantigen SS-A/Ro) |
| 2547 | transcription factor 4 |
| 2548 | hypothetical protein FLJ13868 |
| 2549 | tumor necrosis factor receptor superfamily, member 25 |
| 2550 | ninjurin 2 |
| 2551 | H3 histone, family 3B (H3.3B) |
| 2552 | CD79A antigen (immunoglobulin-associated alpha) |
| 2553 | splicing factor, arginine/serine-rich 14 |
| 2554 | RAB GTPase activating protein 1 |
| 2555 | CDNA FLJ12247 fis, clone MAMMA1001397 |
| 2556 | KIAA1033 protein |
| 2557 | immunoglobulin lambda joining 3 /// immunoglobulin lambda constant 2 (Kern-Oz-marker) |
| 2558 | calcium channel, voltage-dependent, alpha 2/delta subunit 2 |
| 2559 | resistin |
| 2560 | sodium channel, voltage-gated, type III, alpha |
| 2561 | RAB32, member RAS oncogene family |
| 2562 | mitogen-activated protein kinase-activated protein kinase 3 |
| 2563 | mitofusin 2 |
| 2564 | glutathione peroxidase 7 |
| 2565 | — |
| 2566 | uncharacterized hypothalamus protein HT008 |
| 2567 | bone marrow stromal cell antigen 1 |
| 2568 | trinucleotide repeat containing 11 (THR-associated protein, 230 kDa subunit) |
| 2569 | CGI-146 protein |
| 2570 | butyrophilin, subfamily 3, member A3 |
| 2571 | dynamin 1 |
| 2572 | DEAD (Asp-Glu-Ala-Asp) box polypeptide 18 |
| 2573 | MCM7 minichromosome maintenance deficient 7 (S. cerevisiae) |
| 2574 | ATPase, H+ transporting, lysosomal accessory protein 2 |
| 2575 | CDNA FLJ38835 fis, clone MESAN2002424 |
| 2576 | nucleobindin 1 |
| 2577 | lysosomal-associated membrane protein 2 |
| 2578 | protein tyrosine phosphatase, non-receptor type 18 (brain-derived) |
| 2579 | solute carrier family 39 (zinc transporter), member 8 |
| 2580 | integrin, alpha M (complement component receptor 3, alpha; also known as CD11b (p170), macrophage antigen alpha |
| | polypeptide) |
| 2581 | ubiquitin specific protease 3 |
| 2582 | enolase 1, (alpha) |
| 2583 | protein regulator of cytokinesis 1 |
| 2584 | SREBP CLEAVAGE-ACTIVATING PROTEIN |
| 2585 | NEFA-interacting nuclear protein NIP30 |
| 2586 | guanine nucleotide binding protein (G protein) alpha 12 |
| 2587 | activated leukocyte cell adhesion molecule |
| 2588 | chondroitin sulfate proteoglycan 2 (versican) |
| 2589 | special AT-rich sequence binding protein 1 (binds to nuclear matrix/scaffold-associating DNA's) |
| 2590 | HIV-1 Tat interactive protein 2, 30 kDa |
| 2591 | adenosylmethionine decarboxylase 1 |
| 2592 | phosphoinositide-3-kinase, catalytic, beta polypeptide |
| 2593 | inhibitor of kappa light polypeptide gene enhancer in B-cells, kinase beta |
| 2594 | TCDD-inducible poly(ADP-ribose) polymerase |
| 2595 | tumor necrosis factor (ligand) superfamily, member 12-member 13 /// tumor necrosis factor (ligand) superfamily, member 13 |
| 2596 | zinc finger protein 532 |
| 2597 | CD59 antigen p18-20 (antigen identified by monoclonal antibodies 16.3A5, EJ16, EJ30, EL32 and G344) |
| 2598 | EphB4 |
| 2599 | splicing factor, arginine/serine-rich 14 |
| 2600 | adrenergic, beta, receptor kinase 2 |
| 2601 | CTD (carboxy-terminal domain, RNA polymerase II, polypeptide A) phosphatase, subunit 1 |
| 2602 | eukaryotic translation initiation factor 3, subunit 10 theta, 150/170 kDa |
| 2603 | — |
| 2604 | kinetochore associated 2 |
| 2605 | zinc finger protein 227 |
| 2606 | septin 6 |
| 2607 | polyglutamine binding protein 1 |
| 2608 | G protein-coupled receptor 107 |
| 2609 | dual specificity phosphatase 3 (vaccinia virus phosphatase VH1-related) |
| 2610 | MCM5 minichromosome maintenance deficient 5, cell division cycle 46 (S. cerevisiae) |
| 2611 | ATPase, H+ transporting, lysosomal 70 kDa, V1 subunit A |
| 2612 | HBxAg transactivated protein 2 |
| 2613 | zinc finger protein 580 |
| 2614 | collagen-like tail subunit (single strand of homotrimer) of asymmetric acetylcholinesterase |
| 2615 | leucine-rich PPR-motif containing /// leucine-rich PPR-motif containing |
| 2616 | Similar to apolipoprotein B48 receptor |
| 2617 | colony stimulating factor 2 receptor, alpha, low-affinity (granulocyte-macrophage) |
| 2618 | protein kinase C, alpha |
| 2619 | MpV17 transgene, murine homolog, glomerulosclerosis |
| 2620 | tripartite motif-containing 44 |
| 2621 | — |
| 2622 | weakly similar to zinc finger protein 195 |
| 2623 | ret finger protein-like 1 antisense /// ret finger protein-like 3 antisense |
| 2624 | calpastatin |
| 2625 | proline-serine-threonine phosphatase interacting protein 2 |
| 2626 | phosphonoformate immuno-associated protein 5 |
| 2627 | Bruton agammaglobulinemia tyrosine kinase |
| 2628 | arginyl-tRNA synthetase |
| 2629 | programmed cell death 8 (apoptosis-inducing factor) |
| 2630 | dual specificity phosphatase 3 (vaccinia virus phosphatase VH1-related) |
| 2631 | SKI interacting protein |
| 2632 | SFRS protein kinase 2 |
| 2633 | Fc fragment of IgE, low affinity II, receptor for (CD23A) |
| 2634 | hypothetical protein FLJ20699 |
| 2635 | CDP-diacylglycerol synthase (phosphatidate cytidylyltransferase) 2 |
| 2636 | purinergic receptor P2X, ligand-gated ion channel, 7 |
| 2637 | histone 1, H2be |
| 2638 | nuclear receptor coactivator 3 |
| 2639 | Chediak-Higashi syndrome 1 |
| 2640 | CDNA: FLJ21670 fis, clone COL09010 |
| 2641 | zinc finger, ZZ domain containing 3 |
| 2642 | — |
| 2643 | mitochondrial ribosomal protein S12 |
| 2644 | zinc finger protein 292 |
| 2645 | TBC1 (tre-2/USP6, BUB2, cdc16) domain family, member 1 |
| 2646 | glycosyltransferase 25 domain containing 2 |
| 2647 | isocitrate dehydrogenase 3 (NAD+) beta |
| 2648 | apoptosis antagonizing transcription factor |
| 2649 | interleukin enhancer binding factor 3, 90 kDa |
| 2650 | ankyrin repeat domain 17 |
| 2651 | chromosome 9 open reading frame 28 |
| 2652 | hypothetical protein FLJ20729 |
| 2653 | fibrillin 2 (congenital contractural arachnodactyly) |
| 2654 | p66 alpha |
| 2655 | ring finger protein 138 |
| 2656 | dynein, cytoplasmic, light intermediate polypeptide 1 |
| 2657 | — |
| 2658 | urotensin 2 |
| 2659 | transposon-derived Buster1 transposase-like protein gene |
| 2660 | RNA binding motif protein 16 |
| 2661 | transcription factor CP2 |
| 2662 | prolyl endopeptidase |
| 2663 | iduronate 2-sulfatase (Hunter syndrome) |
| 2664 | — |
| 2665 | tyrosine 3-monooxygenase/tryptophan 5-monooxygenase activation protein, beta polypeptide |
| 2666 | UBX domain containing 1 |
| 2667 | protein phosphatase 1F (PP2C domain containing) |
| 2668 | — |
| 2669 | immunoglobulin heavy constant gamma 1 (G1m marker) /// immunoglobulin heavy constant gamma 1 (G1m marker) |
| 2670 | BUB3 budding uninhibited by benzimidazoles 3 homolog (yeast) |
| 2671 | RAN binding protein 10 |
| 2672 | zinc finger, DHHC domain containing 13 |
| 2673 | protein phosphatase 1, regulatory (inhibitor) subunit 15A |
| 2674 | homeodomain interacting protein kinase 1 |
| 2675 | cut-like 1, CCAAT displacement protein (Drosophila) |
| 2676 | CD163 antigen |
| 2677 | low density lipoprotein-related protein 1 (alpha-2-macroglobulin receptor) |
| 2678 | major histocompatibility complex, class II DR beta 3 |
| 2679 | metal-regulatory transcription factor 1 |
| 2680 | tumor necrosis factor (ligand) superfamily, member 12-member 13 /// tumor necrosis factor (ligand) superfamily, member 13 |
| 2681 | receptor-interacting serine-threonine kinase 2 |
| 2682 | CDNA FLJ12105 fis, clone HEMBB1002699 |
| 2683 | karyopherin alpha 2 (RAG cohort 1, importin alpha 1) /// karyopherin alpha 2 (RAG cohort 1, importin alpha 1) |
| 2684 | opsin 3 (encephalopsin, panopsin) |
| 2685 | neuropilin (NRP) and tolloid (TLL)-like 2 |
| 2686 | sema domain, immunoglobulin domain (Ig), short basic domain, secreted, (semaphorin) 3C |
| 2687 | RAB20, member RAS oncogene family |
| 2688 | hypothetical protein LOC284021 |
| 2689 | radixin |
| 2690 | HSPC049 protein |
| 2691 | forkhead box J3 |
| 2692 | integrin-linked kinase |
| 2693 | SWI/SNF related, matrix associated, actin dependent regulator of chromatin, subfamily c, member 2 |
| 2694 | derlin-1 |
| 2695 | hypothetical protein BC002926 |
| 2696 | ribosomal protein S6 kinase, 70 kDa, polypeptide 1 |
| 2697 | RCE1 homolog, prenyl protein protease (S. cerevisiae) |
| 2698 | Immunoglobulin lambda light chain variable and constant region mRNA, partial cds |
| 2699 | exportin, tRNA (nuclear export receptor for tRNAs) |
| 2700 | lysosomal associated protein transmembrane 4 beta /// lysosomal associated protein transmembrane 4 beta |
| 2701 | carboxypeptidase D |
| 2702 | calcineurin binding protein 1 |
| 2703 | intersectin 1 (SH3 domain protein) |
| 2704 | calcium binding protein P22 |
| 2705 | copine I |
| 2706 | KDEL (Lys-Asp-Glu-Leu) endoplasmic reticulum protein retention receptor 2 |
| 2707 | elongation factor RNA polymerase II |
| 2708 | ring finger protein 141 |
| 2709 | KIAA0931 protein |
| 2710 | intercellular adhesion molecule 5, telencephalin |
| 2711 | small optic lobes homolog (Drosophila) |
| 2712 | nucleophosmin (nucleolar phosphoprotein B23, numatrin) /// nucleophosmin (nucleolar phosphoprotein B23, numatrin) |
| 2713 | RAD23 homolog A (S. cerevisiae) |
| 2714 | activating signal cointegrator 1 complex subunit 1 |
| 2715 | AT rich interactive domain 1A (SWI-like) |
| 2716 | HCF-binding transcription factor Zhangfei |
| 2717 | ataxin 10 |
| 2718 | 2,3-bisphosphoglycerate mutase |
| 2719 | ataxia telangiectasia and Rad3 related |
| 2720 | — |
| 2721 | solute carrier family 26, member 6 |
| 2722 | major histocompatibility complex, class II, DM alpha |
| 2723 | mitogen-activated protein kinase kinase kinase 1 |
| 2724 | myotubularin 1 |
| 2725 | phosphatidylinositol-4-phosphate 5-kinase, type II, alpha |
| 2726 | acylphosphatase 1, erythrocyte (common) type |
| 2727 | sulfide quinone reductase-like (yeast) |
| 2728 | pre-B-cell leukemia transcription factor 3 |
| 2729 | chromosome 21 open reading frame 66 |
| 2730 | ceroid-lipofuscinosis, neuronal 3, juvenile (Batten, Spielmeyer-Vogt disease) |
| 2731 | egf-like module containing, mucin-like, hormone receptor-like 1 |
| 2732 | S-phase response (cyclin-related) |
| 2733 | F-box protein 21 |
| 2734 | histone deacetylase 5 |
| 2735 | KIAA1219 protein |
| 2736 | AF-1 specific protein phosphatase /// AF-1 specific protein phosphatase |
| 2737 | zinc finger protein 131 (clone pHZ-10) |
| 2738 | MAP/microtubule affinity-ragulating kinase 2 |
| 2739 | Immunoglobulin kappa light chain VKJ region mRNA, partial cds |
| 2740 | RAB11 family interacting protein 2 (class I) |
| 2741 | — |
| 2742 | WD repeat domain 13 |
| 2743 | oral-facial-digital syndrome 1 |
| 2744 | hypothetical protein FLJ20628 /// hypothetical protein FLJ20628 |
| 2745 | abhydrolase domain containing 5 |
| 2746 | superoxide dismutase 2, mitochondrial |
| 2747 | KIAA0355 |
| 2748 | CDNA FLJ1379 fis, clone THYRO1000026 |
| 2749 | ELAV (embryonic lethal, abnormal vision, Drosophila)-like 3 (Hu antigen C) |
| 2750 | myeloid/lymphoid or mixed-lineage leukemia (trithorax homolog, Drosophila); translocated to, 7 |
| 2751 | F-box protein 42 |
| 2752 | nexilin (F actin binding protein) |
| 2753 | upstream binding transcription factor, RNA polymerase I |
| 2754 | ras homolog gene family, member T2 |
| 2755 | hypothetical protein LOC339287 |
| 2756 | cyclin-dependent kinase inhibitor 2B (p15, inhibits CDK4) |
| 2757 | additional sex combs like 1 (Drosophila) |
| 2758 | cell division cycle 25B |
| 2759 | ubiquitin associated protein 2-like |
| 2760 | RAB GTPase activating protein 1-like |
| 2761 | checkpoint suppressor 1 |
| 2762 | STE20-like kinase |
| 2763 | RIO kinase 3 (yeast) |
| 2764 | phosphodiesterase 4D, cAMP-specific (phosphodiesterase E3 dunce homolog, Drosophila) |
| 2765 | — |
| 2766 | general transcription factor II, i /// general transcription factor II, i, pseudogene 1 |
| 2767 | SWI/SNF related, matrix associated, actin dependent regulator of chromatin, subfamily a, member 2 |
| 2768 | isoleucine-tRNA synthetase |
| 2769 | catenin, beta like 1 /// catenin, beta like 1 |
| 2770 | ubiquitin-conjugating enzyme E2D 3 (UBC4/5 homolog, yeast) |
| 2771 | heat shock 70 kDa protein 6 (HSP70B′) |
| 2772 | translocase of inner mitochondrial membrane 13 homolog (yeast) |
| 2773 | SMART/HDAC1 associated repressor protein |
| 2774 | heterogeneous nuclear ribonucleoprotein A3 |
| 2775 | high-mobility group 20B |
| 2776 | transcription factor 3 (E2A immunoglobulin enhancer binding factors E12/E47) |
| 2777 | DEAD (Asp-Glu-Ala-Asp) box polypeptide 1 |
| 2778 | additional sex combs like 1 (Drosophila) |
| 2779 | myosin, heavy polypeptide 3, skeletal muscle, embryonic |
| 2780 | chromosome 20 open reading frame 3 |
| 2781 | vimentin |
| 2782 | ATPase, Ca++ transporting, ubiquitous |
| 2783 | hypothetical protein FLJ11795 |
| 2784 | interleukin 15 |
| 2785 | neuropeptide FF-amide peptide precursor |
| 2786 | nuclear factor I/B |
| 2787 | Clone 23872 mRNA sequence |
| 2788 | adenylate kinase 2 |
| 2789 | DNA segment on chromosome X (unique) 9928 expressed sequence |
| 2790 | transcription elongation regulator 1 |
| 2791 | per1-like domain containing 1 |
| 2792 | I(3)mbt-like (Drosophila) |
| 2793 | Notch homolog 1, translocation-associated (Drosophila) |
| 2794 | Ras and Rab interactor 3 |
| 2795 | golgi associated gamma adaptin ear containing, ARF binding protein 1 |
| 2796 | chromosome 10 open reading frame 56 |
| 2797 | membrane cofactor protein (CD46, trophoblast-lymphocyte cross-reactive antigen) |
| 2798 | phosphoribosyl pyrophosphate synthetase-associated protein 2 |
| 2799 | poly(A) polymerse alpha |
| 2800 | H2A histone family, member Y |
| 2801 | Meis1, myeloid ecotropic viral integration site 1 homolog 4 (mouse) |
| 2802 | interferon, gamma-inducible protein 30 |
| 2803 | apoptosis inhibitor 5 |
| 2804 | spermidine/spermine N1-acetyltransferase |
| 2805 | golgi reassembly stacking protein 1, 65 kDa |
| 2806 | DiGeorge syndrome critical region gene 8 |
| 2807 | endothelial differentiation, lysophosphatidic acid G-protein-coupled receptor, 2 |
| 2808 | adrenergic, alpha-2A-, receptor |
| 2809 | BCR downstream signaling 1 |
| 2810 | sin3-associated polypeptide, 30 kDa |
| 2811 | filamin B, beta (actin binding protein 278) |
| 2812 | — |
| 2813 | similar to S. cerevisiae SSM4 |
| 2814 | — |
| 2815 | polymerase (RNA) III (DNA directed) polypeptide E (80 kD) |
| 2816 | DKFZP564M182 protein |
| 2817 | phosphatidylinositol glycan, class A (paroxysmal nocturnal hemoglobinuria) |
| 2818 | amyloid beta (A4) precursor protein-binding, family A, member 2 (X11-like) |
| 2819 | SEC5-like 1 (S. cerevisiae) |
| 2820 | glyceronephosphate O-acyltransferase |
| 2821 | hypothetical protein H41 |
| 2822 | SUMO1/sentrin specific protease 6 |
| 2823 | actin, alpha 2, smooth muscle, aorta |
| 2824 | B-cell CLL/lymphoma 11A (zinc finger protein) |
| 2825 | protein kinase C, nu /// protein kinase C, nu |
| 2826 | arachidonate 5-lipoxygenase |
| 2827 | small nuclear ribonucleoprotein polypeptide N |
| 2828 | KIAA1536 protein |
| 2829 | telomeric repeat binding factor 2 |
| 2830 | solute carrier family 35, member E2 |
| 2831 | caveolin 2 |
| 2832 | myeloid/lymphoid or mixed-lineage leukemia (trithorax homolog, Drosophila) |
| 2833 | dimethylarginine dimethylaminohydrolase 2 |
| 2834 | PCTAIRE protein kinase 2 |
| 2835 | pecanex homolog (Drosophila) |
| 2836 | discs, large homolog 1 (Drosophila) |
| 2837 | nuclear receptor subfamily 3, group C, member 1 (glucocorticoid receptor) /// nuclear receptor subfamily 3, group C, member 1 |
| | (glucocorticoid receptor) |
| 2838 | chromosome 15 open reading frame 15 |
| 2839 | Hermansky-Pudlak syndrome 4 |
| 2840 | myosin phosphatase-Rho interacting protein |
| 2841 | malic enzyme 2, NAD(+)-dependent, mitochondrial |
| 2842 | formyl peptide receptor-like 1 |
| 2843 | RAB6A, member RAS oncogene family |
| 2844 | mitochondrial carrier triple repeat 1 |
| 2845 | IGL mRNA for immunoglobulin lambda light chain VLJ region, partial cds, clone: kh0002I |
| 2846 | hypothetical protein PP1057 /// hypothetical protein PP1057 |
| 2847 | bicaudal D homolog 2 (Drosophila) |
| 2848 | Ras-related GTP binding D |
| 2849 | transcription elongation factor A (SII), 1 |
| 2850 | HLA-B associated transcript 8 |
| 2851 | N-deacetylase/N-sulfotransferase (heparan glucosaminyl) 2 |
| 2852 | cutC copper transporter homolog (E. coli) |
| 2853 | synovial sarcoma translocation, chromosome 18 |
| 2854 | clathrin, light polypeptide (Lcb) |
| 2855 | transcriptional intermediary factor 1 |
| 2856 | Notch homolog 2 (Drosophila) |
| 2857 | DEAD (Asp-Glu-Ala-Asp) box polypeptide 18 |
| 2858 | dedicator of cytokinesis 2 |
| 2859 | ELAV (embryonic lethal, abnormal vision, Drosophila)-like 1 (Hu antigen R) |
| 2860 | differentially expressed in FDCP 6 homolog (mouse) |
| 2861 | tumor rejection antigen (gp96) 1 |
| 2862 | G-protein signalling modulator 2 (AGS3-like, C. elegans) |
| 2863 | lysosomal-associated protein transmembrane 4 alpha |
| 2864 | colony stimulating factor 2 receptor, alpha, low-affinity (granulocyte-macrophage) |
| 2865 | high-mobility group box 1 |
| 2866 | forkhead box J2 |
| 2867 | nucleoporin 62 kDa |
| 2868 | glycoprotein Ib (platelet), beta polypeptide /// peanut-like 1 (Drosophila) |
| 2869 | alpha thalassemia/mental retardation syndrome X-linked (RAD54 homolog, S. cerevisiae) |
| 2870 | G protein-coupled receptor 89 |
| 2871 | splicing factor, arginine/serine-rich 14 |
| 2872 | kinesin family member 13A |
| 2873 | transmembrane 9 superfamily member 1 |
| 2874 | spermine oxidase |
| 2875 | suppressor of Ty 4 homolog 1 (S. cerevisiae) |
| 2876 | chromosome 2 open reading frame 17 |
| 2877 | tubulin-specific chaperone d /// tubulin-specific chaperone d |
| 2878 | hypothetical protein LOC283970 |
| 2879 | glucosamine (N-acetyl)-6-sulfatase (Sanfilippo disease IIID) |
| 2880 | solute carrier family 4, sodium bicarbonate cotransporter, member 7 |
| 2881 | annexin A2 |
| 2882 | decay accelerating factor for complement (CD55, Cromer blood group system) |
| 2883 | polypyrimidine tract binding protein 1 |
| 2884 | phosphatase and tensin homolog (mutated in multiple advanced cancers 1) |
| 2885 | — |
| 2886 | thioredoxin domain containing 7 (protein disulfide isomerase) |
| 2887 | malic enzyme 1, NADP(+)-dependent, cytosolic |
| 2888 | forkhead box K1 |
| 2889 | cold shock domain protein A |
| 2890 | solute carrier family 1 (glutamate/neutral amino acid transporter), member 4 |
| 2891 | nuclear factor I/B |
| 2892 | F-box protein 9 |
| 2893 | TRAF and TNF receptor associated protein |
| 2894 | GABA(A) receptor-associated protein-like 2 |
| 2895 | mitochondrial ribosomal protein L9 /// mitochondrial ribosomal protein L9 |
| 2896 | platelet derived growth factor D |
| 2897 | galactosylceramidase (Krabbe disease) |
| 2898 | myeloid leukemia factor 2 |
| 2899 | immediate early response 3 |
| 2900 | carboxypeptidase D |
| 2901 | mannose-6-phosphate receptor (cation dependent) |
| 2902 | tumor rejection antigen (gp96) 1 |
| 2903 | fatty acid binding protein 2, intestinal |
| 2904 | small nuclear ribonucleoprotein polypeptides B and B1 |
| 2905 | proteasome (prosome, macropain) 26S subunit, non-ATPase, 5 |
| 2906 | squamous cell carcinoma antigen recognized by T cells 2 |
| 2907 | retinoblastoma-associated protein 140 |
| 2908 | CD59 antigen p18-20 (antigen identified by monoclonal antibodies 16.3A5, EJ16, EJ30, EL32 and G344) |
| 2909 | regulator of nonsense transcripts 1 |
| 2910 | splicing factor, arginine/serine-rich 2 |
| 2911 | B1 for mucin |
| 2912 | activated Cdc42-associated kinase 1 |
| 2913 | protein kinase C binding protein 1 |
| 2914 | nuclear transcription factor Y, gamma |
| 2915 | mitogen-activated protein kinase kinase 4 |
| 2916 | KIAA0831 |
| 2917 | methyl CpG binding protein 2 (Rett syndrome) |
| 2918 | protein kinase C, theta |
| 2919 | Williams Beuren syndrome chromosome region 14 |
| 2920 | caspase 1, apoptosis-related cysteine protease (interleukin 1, beta, convertase) |
| 2921 | secretory carrier membrane protein 1 |
| 2922 | solute carrier family 39 (zinc transporter), member 8 |
| 2923 | plasma glutamate carboxypeptidase |
| 2924 | SWI/SNF related, matrix associated, actin dependent regulator of chromatin, subfamily a, member 4 |
| 2925 | nucleolar and coiled-body phosphoprotein 1 |
| 2926 | solute carrier family 12 (potassium/chloride transporters), member 7 |
| 2927 | asparaginase like 1 |
| 2928 | adaptor-related protein complex 2, beta 1 subunit |
| 2929 | ARF protein |
| 2930 | KIAA0882 protein |
| 2931 | RNA binding motif protein 19 |
| 2932 | chloride channel 3 |
| 2933 | tubulin, beta, 4 |
| 2934 | ubiquinol-cytochrome c reductase core protein II |
| 2935 | WD repeat domain 37 |
| 2936 | E74-like factor 2 (ets domain transcription factor) |
| 2937 | calcium binding protein 39 |
| 2938 | polymerase (RNA) II (DNA directed) polypeptide A, 220 kDa |
| 2939 | guanine nucleotide binding protein (G protein), beta polypeptide 2-like 1 |
| 2940 | aspartyl-tRNA synthetase |
| 2941 | UDP-N-acetyl-alpha-D-galactosamine:polypeptide N-acetylgalactosaminyltransferase 1 (GalNAc-T1) |
| 2942 | calpain 2, (m/II) large subunit |
| 2943 | SET translocation (myeloid leukemia-associated) |
| 2944 | LanC lantibiotic synthetase component C-like 2 (bacterial) |
| 2945 | eukaryotic translation initiation factor 5 |
| 2946 | nebulette |
| 2947 | serologically defined colon cancer antigen 8 |
| 2948 | cathepsin W (lymphopain) |
| 2949 | ubiquitin-like 3 |
| 2950 | major histocompatibility complex, class II, DR beta 4 |
| 2951 | MAX protein |
| 2952 | kinetochore associated 1 |
| 2953 | lectin, galactoside-binding, soluble, 8 (galectin 8) |
| 2954 | mucosa associated lymphoid tissue lymphoma translocation gene 1 |
| 2955 | methyl-CpG binding domain protein 4 |
| 2956 | BCL2 binding component 3 /// BCL2 binding component 3 |
| 2957 | sialyltransferase 40 (beta-galactoside alpha-2,3-sialyltransferase) |
| 2958 | tensin |
| 2959 | major histocompatibility complex, class II, DP beta 1 |
| 2960 | golgi associated, gamma adaptin ear containing, ARF binding protein 2 |
| 2961 | zinc finger protein 297 |
| 2962 | malic enzyme 2, NAD(+)-dependent, mitochondrial |
| 2963 | zinc finger protein 45 (a Kruppel-associated box (KRAB) domain polypeptide) |
| 2964 | eukaryotic translation initiation factor 4 gamma, 1 |
| 2965 | karyopherin alpha 1 (importin alpha 5) |
| 2966 | electron-transferring-flavoprotein dehydrogenase |
| 2967 | ZW10 interactor |
| 2968 | Full-length cDNA clone CS0DI052YI05 of Placenta Cot 25-normalized of Homo sapiens (human) |
| 2969 | solute carrier family 25, member 13 (citrin) |
| 2970 | GDP dissociation inhibitor 1 |
| 2971 | hypothetical protein FLJ22419 |
| 2972 | thyroid hormone receptor interactor 12 |
| 2973 | neuralized-like (Drosophila) |
| 2974 | rTS beta protein |
| 2975 | mitofusin 1 |
| 2976 | calpastatin |
| 2977 | SPTF-associated factor 65 gamma |
| 2978 | — |
| 2979 | filamin B, beta (actin binding protein 278) |
| 2980 | apoptosis inhibitor 5 |
| 2981 | RAB27A, member RAS oncogene family |
| 2982 | KIAA0317 |
| 2983 | cyclin-dependent kinase inhibitor 2A (melanoma, p16, inhibits CDK4) |
| 2984 | acid phosphatase, prostate |
| 2985 | unc-93 homolog B1 (C. elegans) /// unc-93 homolog B1 (C. elegans) |
| 2986 | ubiqutin specific protease 16 |
| 2987 | NSFL1 (p97) cofactor (p47) |
| 2988 | integrin, beta 7 |
| 2989 | chromosome 9 open reading frame 60 |
| 2990 | importin 7 |
| 2991 | granulin |
| 2992 | Tu translation elongation factor, mitochondrial |
| 2993 | phospholipase A2, group IVB (cytosolic) |
| 2994 | amyloid beta precursor protein (cytoplasmic tail) binding protein 2 |
| 2995 | nucleoporin like 1 |
| 2996 | aspartyl-tRNA synthetase |
| 2997 | TRAF family member-associated NFKB activator |
| 2998 | KIAA0182 protein |
| 2999 | protein kinase, AMP-activated, alpha 1 catalytic subunit |
| 3000 | ATPase, H+ transporting, lysosomal 21 kDa, V0 subunit c″ /// ATPase, H+ transporting, lysosomal 21 kDa, V0 subunit c″ |
| 3001 | Clone 24889 mRNA sequence |
| 3002 | transitional epithelia response protein |
| 3003 | leukocyte immunoglobulin-like receptor, subfamily A (with TM domain), member 2 |
| 3004 | solute carrier family 25 (mitochondrial carrier, brain), member 14 |
| 3005 | discs, large homolog 1 (Drosophila) |
| 3006 | protein kinase (cAMP-dependent, catalytic) inhibitor gamma |
| 3007 | transmembrane protein 1 |
| 3008 | vav 3 oncogene |
| 3009 | UDP-N-acetyl-alpha-D-galactosamine: potypeptide N-acetylgalactosaminyltransferase 12 (GalNAc-T12) |
| 3010 | golgi associated, gamma adaptin ear containing, ARF binding protein 2 |
| 3011 | zinc finger, FYVE domain containing 16 |
| 3012 | ras homolog gene family, member G (rho G) |
| 3013 | biliverdin reductase A |
| 3014 | — |
| 3015 | CDC37 cell division cycle 37 homolog (S. carevisiae) |
| 3016 | presenilin 2 (Alzheimer disease 4) |
| 3017 | platelet-activating factor acetylhydrolase, isoform lb, alpha subunit 45 kDa |
| 3018 | apolipoprotein A-IV |
| 3019 | dedicator of cytokinesis 3 |
| 3020 | neurobeachin |
| 3021 | LIM protein (similar to rat protein kinase C-binding enigma) |
| 3022 | mesoderm specific transcript homolog (mouse) |
| 3023 | U2-associated SR140 protein |
| 3024 | regulating synaptic membrane exocytosis 3 |
| 3025 | lectin, galactoside-binding, soluble, 9 (galectin 9) |
| 3026 | DEP domain containing 5 |
| 3027 | leptin receptor overlapping transcript-like 1 |
| 3028 | MLL septin-like fusion |
| 3029 | lipase A, lysosomal acid, cholesterol esterase (Wolman disease) |
| 3030 | EH-domain containing 4 |
| 3031 | C-terminal binding protein 1 |
| 3032 | dual specificity phosphatase 8 |
| 3033 | heat shock 70 kDa protein 8 |
| 3034 | staufen, RNA binding protein (Drosophila) |
| 3035 | adenosine desminase |
| 3036 | chromosome 19 open reading frame 2 |
| 3037 | a disintegrin and metalloproteinase domain 28 |
| 3038 | chromosome 1 open reading frame 24 |
| 3039 | hypothetical protein LOC51321 |
| 3040 | PTD016 protein |
| 3041 | sorting nexin 1 |
| 3042 | FK506 binding protein 1A, 12 kDa |
| 3043 | pleckstrin homology, Sec7 and coiled-coil domains 2 (cytohesin-2) |
| 3044 | cleavage and polyadenylation specific factor 6, 68 kDa |
| 3045 | dynactin 2 (p50) |
| 3046 | SEC24 related gene family, member C (S. cerevisiae) |
| 3047 | — |
| 3048 | microtubule associated serine/threonine kinase 3 |
| 3049 | trinucleotide repeat containing 11 (THR-associated protein, 230 kDa subunit) |
| 3050 | a disintegrin and metalloproteinase domain 19 (meltrin beta) |
| 3051 | hydroxyprostaglandin dehydrogenase 15-(NAD) |
| 3052 | epithelial membrane protein 3 |
| 3053 | POM121 membrane glycoprotein (rat) /// hypothetical protein LOC340318 |
| 3054 | mediator of RNA polymerase II transcription, subunit 8 homolog (yeast) |
| 3055 | developmentally regulated GTP binding protein 1 |
| 3056 | cisplatin resistance-associated overexpressed protein |
| 3057 | ring finger protein 103 |
| 3058 | vesicle-associated membrane protein 1 (synaptobrevin 1) |
| 3059 | BCL2-associated athanogene 5 |
| 3060 | inositol 1,4,5-triphosphate receptor, type 2 |
| 3061 | zinc finger protein 281 |
| 3062 | small nuclear ribonucleoprotein D1 polypeptide 16 kDa |
| 3063 | D component of complement (adipsin) |
| 3064 | KIAA1117 |
| 3065 | amplified in osteosarcoma |
| 3066 | sterol carrier protein 2 |
| 3067 | beta 5-tubulin /// beta 5-tubulin |
| 3068 | hypothetical protein FLJ20421 |
| 3069 | active BCR-related gene |
| 3070 | — |
| 3071 | DKFZP586J0619 protein |
| 3072 | pre-B-cell leukemia transcription factor 1 |
| 3073 | solute carrier family 2 (facilitated glucose transporter), member 6 |
| 3074 | dentin sialophosphoprotein |
| 3075 | cystatin F (leukocystatin) |
| 3076 | mannosyl (alpha-1,6-)-glycoprotein beta-1,2-N-acetylglucosaminyltransferase /// mannosyl (alpha-1,6-)-glycoprotein beta-1,2-N- |
| 3077 | CREBBP/EP300 inhibitor 1 |
| 3078 | peroxiredoxin 6 |
| 3079 | thymopoietin |
| 3080 | presenilin associated, rhomboid-like |
| 3081 | chromodomain protein, Y-like |
| 3082 | hypothetical protein FLJ10154 |
| 3083 | eukaryotic translation initiation factor 5 |
| 3084 | syntaxin 16 |
| 3085 | GDP dissociation inhibitor 2 /// GDP dissociation inhibitor 2 |
| 3086 | mitogen-activated protein kinase kinase kinase 3 |
| 3087 | KIAA0100 gene product |
| 3088 | annexin A2 |
| 3089 | CCAAT/enhancer binding protein (C/EBP), epsilon |
| 3090 | polymerase (RNA) II (DNA directed) polypeptide B, 140 kDa |
| 3091 | calumenin |
| 3092 | WD repeat domain 42A |
| 3093 | core-binding factor, beta subunit |
| 3094 | hect domain and RLD 4 |
| 3095 | RecQ protein-like 5 |
| 3096 | steroid sulfatase (microsomal), arylsulfatase C, isozyme S |
| 3097 | ADP-ribosylation-like factor 6 interacting protein 5 |
| 3098 | titin |
| 3099 | — |
| 3100 | peptidylglycine alpha-amidating monooxygenase |
| 3101 | MRNA; cDNA DKFZp434L092 (from clone DKFZp434L092) |
| 3102 | Chromosome 21q22.1 anonymous mRNA sequence |
| 3103 | mutS homolog 3 (E. coli) |
| 3104 | kinesin family member 13B |
| 3105 | v-akt murine thymoma viral oncogene homolog 1 |
| 3106 | hypothetical protein FLJ11286 |
| 3107 | thyroid hormone receptor associated protein 2 |
| 3108 | chromodomain protein, Y-like |
| 3109 | septin 6 |
| 3110 | HESB like domain containing 2 |
| 3111 | megakaryocyte-associated tyrosine kinase |
| 3112 | zinc finger protein 198 |
| 3113 | growth factor independent 1 |
| 3114 | two pore segment channel 1 |
| 3115 | transmembrane protein 2 |
| 3116 | immunoglobulin heavy constant mu |
| 3117 | adaptor-related protein complex 2, alpha 2 subunit |
| 3118 | bromodomain containing 1 |
| 3119 | glycoprotein Ib (platelet), beta polypeptide |
| 3120 | growth arrest-specific 7 |
| 3121 | cytosolic ovarian carcinoma antigen 1 |
| 3122 | ret finger protein 2 |
| 3123 | secretin |
| 3124 | nucleolar and spindle associated protein 1 |
| 3125 | basic transcription element binding protein 1 |
| 3126 | guanine nucleotide binding protein (G protein), alpha 15 (Gq class) |
| 3127 | peroxiredoxin 6 |
| 3128 | four and a half LIM domains 1 |
| 3129 | cathepsin B |
| 3130 | trinucleotide repeat containing 4 |
| 3131 | — |
| 3132 | APG4 autophagy 4 homolog B (S. cerevisiae) |
| 3133 | — |
| 3134 | integrin beta 4 binding protein |
| 3135 | KIAA0020 |
| 3136 | chromosome 9 open reading frame 28 |
| 3137 | insulin-like growth factor 2 receptor |
| 3138 | S-adenosylhomocysteine hydrolase-like 1 |
| 3139 | WD repeat domain 7 |
| 3140 | chromosome X open reading frame 37 |
| 3141 | chromosome 20 open reading frame 24 |
| 3142 | leukocyte immunoglobulin-like receptor, subfamily A (with TM domain), member 1 |
| 3143 | ubiquitously transcribed tetratricopeptide repeat gene, Y-linked |
| 3144 | CD99 antigen |
| 3145 | golgi autoantigen, golgin subfamily a, 1 |
| 3146 | diaphanous homolog 2 (Drosophila) |
| 3147 | SH2 domain protein 1A, Duncan's disease (lymphoproliferative syndrome) |
| 3148 | KIAA0913 |
| 3149 | cytidine monophosphate-N-acetylneuraminic acid hydroxylase (CMP-N-acetylneuraminate monooxygenase) |
| 3150 | bobby sox homolog (Drosophila) |
| 3151 | nucleoporin 160 kDa |
| 3152 | 5-methyltetrahydrofolate-homocysteine methyltransferase reductase |
| 3153 | Similar to hypothetical protein KIAA0454 - human (fragment) |
| 3154 | RIO kinase 3 (yeast) |
| 3155 | hypothetical protein FLJ22573 |
| 3156 | MADS box transcription enhancer factor 2, potypeptide A (myocyte enhancer factor 2A) |
| 3157 | — |
| 3158 | PTEN induced putative kinase 1 |
| 3159 | synaptonemal complex protein 2 |
| 3160 | myeloid cell nuclear differentiation antigen |
| 3161 | major histocompatibility complex, class II, DM beta |
| 3162 | ubiquitin specific protease 19 |
| 3163 | adenylate cyclase 9 |
| 3164 | serum/glucocorticoid regulated kinase-like |
| 3165 | MAP/microtubule affinity-regulating kinase 3 |
| 3166 | down-regulator of transcription 1, TBP-binding (negative cofactor 2) |
| 3167 | chromosome 22 open reading frame 4 |
| 3168 | cAMP responsive element binding protein 1 |
| 3169 | lactate dehydrogenase A |
| 3170 | tyrosine 3-monooxygenase/tryptophan 5-monooxygenase activation protein, epsilon polypeptide |
| 3171 | t-complex-associated-testis-expressed 1-like 1 |
| 3172 | hypothetical protein FLJ22814 |
| 3173 | chromosome 14 open reading frame 87 |
| 3174 | — |
| 3175 | O-sialoglycoprotein endopeptidase |
| 3176 | transforming, acidic coiled-coil containing protein 1 |
| 3177 | ataxin 1 |
| 3178 | far upstream element (FUSE) binding protein 1 |
| 3179 | signal sequence receptor, alpha (translocon-associated protein alpha) |
| 3180 | ferritin, heavy polypeptide 1 |
| 3181 | protein-L-isoaspartate (D-aspartate) O-methyltransferase |
| 3182 | nuclear receptor coactivator 1 |
| 3183 | latent transforming growth factor beta binding protein 4 |
| 3184 | transcription factor 7-like 2 (T-cell specific, HMG-box) |
| 3185 | SEC3-like 1 (S. cerevisiae) |
| 3186 | — |
| 3187 | translocase of outer mitochondrial membrane 20 homolog (yeast) |
| 3188 | zinc finger, DHHC domain containing 7 |
| 3189 | putative ATPase |
| 3190 | SEC31-like 1 (S. cerevisiae) |
| 3191 | chromosome 4 open reading frame 8 |
| 3192 | mitogen-activated protein kinase associated protein 1 |
| 3193 | — |
| 3194 | leucine rich repeat (in FLII) interacting protein 1 |
| 3195 | pre-B-cell leukemia transcription factor 2 |
| 3196 | ubiquitin protein ligase E3A (human papilloma virus E6-associated protein, Angelman syndrome) |
| 3197 | chromosome 13 open reading frame 17 |
| 3198 | C-type (calcium dependent, carbohydrate-recognition domain) lectin, superfamily member 6 |
| 3199 | sparc/osteonectin, cwcv and kazal-like domains proteoglycan (testican) 2 |
| 3200 | ubiquitin-activating enzyme E1 (A1S9T and BN75 temperature sensitivity complementing) |
| 3201 | CD22 antigen |
| 3202 | signal recognition particle 54 kDa |
| 3203 | KIAA0676 protein |
| 3204 | KIAA0492 protein |
| 3205 | KIAA0404 protein |
| 3206 | polymerase (DNA directed), gamma 2, accessory subunit |
| 3207 | — |
| 3208 | heterogeneous nuclear ribonucleoprotein A0 |
| 3209 | — |
| 3210 | CDNA: FLJ20931 fis, clone ADSE01282 |
| 3211 | zinc finger protein 330 |
| 3212 | suppressor of Ty 16 homolog (S. cerevisiae) |
| 3213 | COMM domain containing 3 |
| 3214 | TNF receptor-associated facter 3 |
| 3215 | — |
| 3216 | a disintegrin and metalloproteinase domain 10 |
| 3217 | ADP-ribosylation factor 1 /// ADP-ribosylation factor 1 |
| 3218 | S-phase kinase-associated protein 1A (p19A) |
| 3219 | — |
| 3220 | par-3 partitioning defective 3 homolog (C. elegans) |
| 3221 | sorbitol dehydrogenase |
| 3222 | IQ motif containing GTPase activating protein 1 |
| 3223 | hypothetical protein FLJ10357 |
| 3224 | SON DNA binding protein |
| 3225 | GrpE-like 1, mitochondrial (E. coli) |
| 3226 | — |
| 3227 | CD44 antigen (homing function and Indian blood group system) |
| 3228 | — |
| 3229 | CD24 antigen (small cell lung carcinoma cluster 4 antigen) |
| 3230 | dual specificity phosphatase 1 |
| 3231 | protein inhibitor of activated STAT, 3 |
| 3232 | down-regulator of transcription 1, TBP-binding (negative cofactor 2) |
| 3233 | hypothetical protein DKFZp434B227 |
| 3234 | nuclear receptor coactivator 1 |
| 3235 | DKFZP564G2022 protein |
| 3236 | DnaJ (Hsp40) homolog, subfamily C, member 9 |
| 3237 | hypothetical protein DKFZp761I2123 |
| 3238 | SEC23 interacting protein |
| 3239 | proteasome (prosome, macropain) subunit, alpha type, 3 |
| 3240 | ATPase, Na+/K+ transporting, beta 1 polypeptide |
| 3241 | solute carrier family 35, member B1 |
| 3242 | syntaxin binding protein 2 |
| 3243 | estrogen receptor binding protein |
| 3244 | CASP8 and FADD-like apoptosis regulator |
| 3245 | zinc finger CCCH type domain containing 1 |
| 3246 | membrane-bound transcription factor protease, site 1 |
| 3247 | transmembrane trafficking protein |
| 3248 | ubiquitination factor E4B (UFD2 homolog, yeast) |
| 3249 | transmembrane 9 superfamily protein member 4 |
| 3250 | GTPase activating Rap/RanGAP domain-like 1 |
| 3251 | chromosome X open reading frame 40 |
| 3252 | chromodomain helicase DNA binding protein 3 |
| 3253 | catenin (cadherin-associated protein), delta 1 |
| 3254 | — |
| 3255 | NADH dehydrogenase (ubiquinone) 1 alpha subcomplex, 5, 13 kDa |
| 3256 | chromosome 20 open reading frame 13 |
| 3257 | golgi phosphoprotein 4 |
| 3258 | ras homolog gene family, member C |
| 3259 | ubiquitin-conjugating enzyme E2D 1 (UBC4/5 homolog, yeast) /// ubiquitin-conjugating enzyme E2D 1 (UBC4/5 homolog, yeast) |
| 3260 | DEAD (Asp-Glu-Ala-Asp) box polypeptida 50 /// DEAD (Asp-Glu-Ala-Asp) box polypeptide 50 |
| 3261 | hypothetical protein FLJ10350 |
| 3262 | sorting nexin 13 |
| 3263 | chromosome 20 open reading frame 12 |
| 3264 | WD repeat domain 19 |
| 3265 | armadillo repeat gene deletes in velocardiofacial syndrome |
| 3266 | CDNA FLJ10131 fis, clone HEMBA1003041 |
| 3267 | solute carrier family 10 (sodium/bile acid cotransporter family), member 3 |
| 3268 | damage-specific DNA binding protein 2, 48 kDa |
| 3269 | solute carrier family 31 (copper transporters), member 1 |
| 3270 | purinergic receptor P2Y, G-protein coupled, 2 |
| 3271 | M-phase phosphoprotein, mpp8 |
| 3272 | protein kinase D2 |
| 3273 | thousand and one amino acid protein kinase |
| 3274 | nucleoporin 107 kDa |
| 3275 | H2A histone family, member Y |
| 3276 | GTPase activating Rap/RanGAP domain-like 4 |
| 3277 | CXXC finger 1 (PHD domain) |
| 3278 | DKFZP566B183 protein |
| 3279 | transcriptional adaptor 3 (NGG1 homolog, yeast)-like |
| 3280 | urocortin |
| 3281 | — |
| 3282 | carboxypeptidase D |
| 3283 | protein O-fucosyltransferase 2 |
| 3284 | olfactory receptor, family 2, subfamily A, member 20 pseudogene |
| 3285 | phosphoinositide-3-kinase, catalytic, delta polypeptide |
| 3286 | interleukin 6 signal transducer (gp130, oncostatin M receptor) |
| 3287 | tuberous sclerosis 1 |
| 3288 | calmodulin binding transcription activator 1 |
| 3289 | chromosome 2 open reading frame 17 |
| 3290 | exosome component 10 |
| 3291 | ATP citrate lyase |
| 3292 | solute carrier family 5 (inositol transporters), member 3 |
| 3293 | X-ray repair complementing defective repair in Chinese hamster cells 1 |
| 3294 | enoyl Coenzyme A hydratase domain containing 1 |
| 3295 | PRP4 pre-mRNA processing factor 4 homolog B (yeast) |
| 3296 | dCMP deaminase |
| 3297 | fibroblast growth factor receptor 3 (achondroplasia, thanatophoric dwarfism) |
| 3298 | signal transducing adaptor molecule (SH3 domain and ITAM motif) 1 |
| 3299 | F-box and leucine-rich repeat protein 11 |
| 3300 | TGFB inducible early growth response |
| 3301 | SH3-domain binding protein 2 |
| 3302 | phosphatidylinositol-4-phosphate 5-kinase, type I, gamma |
| 3303 | nucleoporin 93 kDa |
| 3304 | SMA4 |
| 3305 | uncharacterized bone marrow protein BM039 |
| 3306 | proteasome (prosome, macropain) subunit alpha type, 6 |
| 3307 | zinc finger, DHHC domain containing 4 |
| 3308 | lamin A/C |
| 3309 | karyopherin (importin) beta 1 |
| 3310 | — |
| 3311 | tRNA isopentenyltransferase 1 |
| 3312 | A kinase (PRKA) anchor protein 2 |
| 3313 | CD8 antigen, alpha polypeptide (p32) |
| 3314 | fumarate hydratase |
| 3315 | pyridoxal (pyridoxine, vitamin B6) kinase |
| 3316 | likely ortholog of mouse immediate early response, erythropoietin 4 |
| 3317 | tumor differentially expressed 2 |
| 3318 | hect (homologous to the E6-AP (UBE3A) carboxyl terminus) domain and RCC1 (CHC1)-like domain (RLD) 1 |
| 3319 | phosphofructokinase, liver |
| 3320 | NAD(P)H dehydrogenase, quinone 2 |
| 3321 | SEC13-like 1 (S. cerevisiae) |
| 3322 | chromosome 14 open reading frame 92 |
| 3323 | coagulation factor VIII-associated (intronic transcript) |
| 3324 | complement factor H-related 1 |
| 3325 | Ras-related GTP binding C |
| 3326 | KIAA0040 gene product |
| 3327 | eukaryotic translation initiation factor 1A, Y-linked |
| 3328 | NADH dehydrogenase (ubiquinone) 1 alpha subcomplex, 10, 42 kDa |
| 3329 | translin |
| 3330 | M-phase phosphoprotein 9 |
| 3331 | phospholipase A2, group IVA (cytosolic, calcium-dependent) |
| 3332 | C-terminal binding protein 1 |
| 3333 | protein phosphatase 2 (formerly 2A), regulatory subunit A (PR 65), alpha isoform |
| 3334 | pericentrin 1 |
| 3335 | craniofacial development protein 1 |
| 3336 | heterogeneous nuclear ribonucleoprotein A0 |
| 3337 | FUS interacting protein (serine-arginine rich) 1 |
| 3338 | zinc finger protein 42 (myeloid-specific retinoic acid-responsive) |
| 3339 | formyl peptide receptor-like 1 |
| 3340 | left-right determination, factor B |
| 3341 | asparagine synthetase |
| 3342 | likely ortholog of rat vacuole membrane protein 1 /// likely ortholog of rat vacuole membrane protein 1 |
| 3343 | RAB4A, member RAS oncogene family |
| 3344 | PTPRF interacting protein, binding protein 2 (liprin beta 2) |
| 3345 | casein kinase 1, alpha 1 |
| 3346 | eukaryotic translation elongation factor 1 epsilon 1 |
| 3347 | sex comb on midleg-like 1 (Drosophila) |
| 3348 | TBC1 domain family, member 12 |
| 3349 | placental growth factor, vascular endothelial growth factor-related protein |
| 3350 | mitogen-activated protein kinase 8 interacting protein 3 |
| 3351 | BUB3 budding uninhibited by benzimidazoles 3 homolog (yeast) |
| 3352 | hypothetical protein FLJ11011 |
| 3353 | CD79B antigen (immunoglobulin-associated beta) |
| 3354 | CDNA: FLJ21256 fis, clone COL01402 |
| 3355 | SWI/SNF related, matrix associated, actin dependent regulator of chromatin, subfamily a, member 4 |
| 3356 | — |
| 3357 | nucleoporin 155 kDa |
| 3358 | peroxiredoxin 4 |
| 3359 | ring finger protein 24 |
| 3360 | interleukin-1 receptor-associated kinase 1 binding protein 1 |
| 3361 | — |
| 3362 | CDNA FLJ11568 fis, clone HEMBA1003278 |
| 3363 | CDNA FLJ12248 fis, clone MAMMA1001408 |
| 3364 | beta-amyloid binding protein precursor /// beta-amyloid binding protein precursor |
| 3365 | FXYD domain containing ion transport regulator 2 |
| 3366 | poly(A) binding protein, cytoplasmic 4 (inducible form) |
| 3367 | chromosome 14 open reading frame 135 |
| 3368 | — |
| 3369 | topoisomerase (DNA) I |
| 3370 | solute carrier family 33 (acetyl-CoA transporter), member 1 |
| 3371 | mutS homolog 2, colon cancer, nonpolyposis type 1 (E. coli) |
| 3372 | cadherin 15, M-cadherin (myotubule) |
| 3373 | LAG1 longevity assurance homolog 2 (S. cerevisiae) |
| 3374 | calmodulin regulated spectrin-associated protein 1 |
| 3375 | — |
| 3376 | tumor suppressing subtransferable candidate 1 |
| 3377 | cytoplasmic linker associated protein 2 |
| 3378 | hypothetical protein FLJ22104 |
| 3379 | cysteinyl-tRNA synthetase |
| 3380 | eukaryotic translation initiation faclor 4B |
| 3381 | diacylglycerol kinase, zeta 104 kDa |
| 3382 | selectin P ligand |
| 3383 | single-stranded DNA binding protein 1 |
| 3384 | centaurin, beta 1 |
| 3385 | mannose-6-phosphate receptor (cation dependent) |
| 3386 | ubiquitin specific protease 34 |
| 3387 | bromodomain containing 1 |
| 3388 | family with sequence similarity 20, member B |
| 3389 | Nipped-B homolog (Drosophila) |
| 3390 | RNA binding motif protein 6 |
| 3391 | polymerase (RNA) III (DNA directed) polypeptide F, 39 kDa |
| 3392 | — |
| 3393 | mercaptopyruvate sulfurtransferase |
| 3394 | estrogen-related receptor beta like 1 |
| 3395 | lectin, galacloside-binding, soluble, 8 (galectin 8) |
| 3396 | ubiquitin protein ligase E3B |
| 3397 | ATPase family homolog up-regulated in senescence cells |
| 3398 | meningioma expressed antigen 5 (hyaluronidase) |
| 3399 | mitogen-activated protein kinase-activated protein kinase 3 |
| 3400 | homeo box B2 |
| 3401 | trafficking protein particle complex 3 |
| 3402 | phospholipase D3 |
| 3403 | TATA box binding protein (TBP)-associated factor, RNA polymerase I, A, 48 kDa |
| 3404 | N-acetylneuraminic acid synthase (sialic acid synthase) |
| 3405 | hypothetical protein LOC126208 |
| 3406 | killer cell lectin-like receptor subfamily D, member 1 |
| 3407 | splicing factor, arginine/serine-rich 11 |
| 3408 | synovial sarcoma, X breakpoint 2 interacting protein |
| 3409 | SH2 domain protein 1A, Duncan's disease (lymphoproliferative syndrome) |
| 3410 | polypyrimidine tract binding protein 1 |
| 3411 | cyclin-dependent kinase inhibitor 2C (p18, inhibits CDK4) |
| 3412 | adenylate kinase 2 |
| 3413 | TBC1 domain family, member 3 |
| 3414 | heterogeneous nuclear ribonucleoprotein R |
| 3415 | collagen, type XVIII, alpha 1 |
| 3416 | voltage-dependent anion channel 1 |
| 3417 | type 1 tumor necrosis factor receptor shedding aminopeptidase regulator |
| 3418 | transportin 3 |
| 3419 | TAF12 RNA polymerase II, TATA box binding protein (TBP)-associated factor, 20 kDa |
| 3420 | synaptogyrin 2 |
| 3421 | nudix (nucleoside diphosphate linked moiety X)-type motif 4 |
| 3422 | ubiquitin specific protease 24 |
| 3423 | ras homolog gene family, member T2 |
| 3424 | bromodomain adjacent to zinc finger domain, 1A |
| 3425 | hypothetical protein FLJ22269 |
| 3426 | — |
| 3427 | zinc finger protein 331 |
| 3428 | adaptor-related protein complex 1, gamma 2 subunit |
| 3429 | cadherin, EGF LAG seven-pass G-type receptor 3 (flamingo homolog, Drosophila) |
| 3430 | methyl-CpG binding domain protein 4 |
| 3431 | — |
| 3432 | Treacher Collins-Franceschetti syndrome 1 |
| 3433 | nuclear phosphoprotein similar to S. cerevisiae PWP1 |
| 3434 | ras homolog gene family, member Q |
| 3435 | ketch-like 7 (Drosophila) |
| 3436 | polyglutamine binding protein 1 |
| 3437 | somatostatin receptor 2 |
| 3438 | KIAA0082 |
| 3439 | WD repeat domain 44 |
| 3440 | karyopherin alpha 3 (importin alpha 4) |
| 3441 | adenylate kinase 2 |
| 3442 | glutathione S-transferase omega 1 |
| 3443 | actin related protein 2/3 complex, subunit 5, 16 kDa |
| 3444 | arginine vasopressin receptor 2 (nephrogenic diabetes insipidus) |
| 3445 | DEAH (Asp-Glu-Ala-His) box polypeptide 38 |
| 3446 | BUB3 budding uninhibited by benzimidazoles 3 homolog (yeast) |
| 3447 | F-box protein 21 |
| 3448 | cylindromatosis (turban tumor syndrome) |
| 3449 | chromosome 19 open reading frame 2 |
| 3450 | pre-B-cell leukemia transcription factor 1 |
| 3451 | TNF receptor-associated factor 3 |
| 3452 | KIAA0286 protein |
| 3453 | — |
| 3454 | Niemann-Pick disease, type C2 |
| 3455 | MRNA; cDNA DKFZp564F133 (from clone DKFZp564F133) |
| 3456 | phosphatidylinositol binding clathrin assembly protein |
| 3457 | E74-like factor 2 (ets domain transcription factor) |
| 3458 | pleckstrin homology, Sec7 and coiled-coil domains 1(cytohesin 1) |
| 3459 | uroporphyrinogen decarboxylase |
| 3460 | interferon gamma receptor 1 |
| 3461 | chromosome 10 open reading frame 56 |
| 3462 | SEC24 related gene family, member B (S. cerevisiae) |
| 3463 | chromosome 18 open reading frame 1 |
| 3464 | cystatin C (amyloid angiopathy and cerebral hemorrhage) |
| 3465 | toll-like receptor 5 |
| 3466 | matrix metalloproteinase 23A /// matrix metalloproteinase 23B |
| 3467 | calcium channel, voltage-dependent, beta 2 subunit |
| 3468 | spondin 2, extracellular matrix protein |
| 3469 | likely ortholog of mouse la related protein |
| 3470 | zinc finger protein 289, ID1 regulated |
| 3471 | Ras-GTPase-activating protein SH3-domain-binding protein |
| 3472 | unc-84 homolog A (C. elegans) |
| 3473 | proteasome (prosome, macropain) 26S subunit, non-ATPase, 14 |
| 3474 | 7-dehydrocholesterol reductase |
| 3475 | chromosome 13 open reading frame 18 |
| 3476 | Chediak-Higashi syndrome 1 |
| 3477 | — |
| 3478 | phosphatidylserine synthase 2 /// phosphatidylserine synthase 2 |
| 3479 | zinc finger protein 161 homolog (mouse) |
| 3480 | solute carrier family 11 (proton-coupled divalent metal ion transporters), member 1 |
| 3481 | CCR4-NOT transcription complex, subunit 3 |
| 3482 | Jumonji, AT rich interactive domain 2 |
| 3483 | solute carrier family 39 (zinc transporter), member 8 |
| 3484 | ubiquitin specific protease 4 (proto-oncogene) |
| 3485 | pan-hematopoietic expression |
| 3486 | coronin, actin binding protein, 1B |
| 3487 | sphingosine-1-phosphate lyase 1 |
| 3488 | myotubularin related protein 1 |
| 3489 | apoptotic protease activating factor |
| 3490 | NHP2 non-histone chromosome protein 2-like 1 (S. cerevisiae) |
| 3491 | syndecan binding protein (syntenin) |
| 3492 | caspase 4, apoptosis-related cysteine protease |
| 3493 | WW domain containing oxidoreductase |
| 3494 | nuclear factor of activated T-cells, cytoplasmic, calcineurin-dependent 1 |
| 3495 | related RAS viral (r-ras) oncogene homolog 2 |
| 3496 | protein phosphatase 2 (formerly 2A), regulatory subunit B (PR 52), alpha isoform |
| 3497 | ataxin 2-like |
| 3498 | lymphotoxin beta receptor (TNFR superfamily, member 3) |
| 3499 | hypothetical protein MGC2654 |
| 3500 | CDC14 cell division cycle 14 homolog A (S. cerevisiae) |
| 3501 | lipoma HMGIC fusion partner-like 2 |
| 3502 | hypothetical protein FLJ10618 |
| 3503 | zinc finger protein 516 |
| 3504 | T cell receptor delta locus |
| 3505 | — |
| 3506 | Lysosomal-associated multispanning membrane protein-5 |
| 3507 | phosphoinositide-3-kinase, catalytic, beta polypeptide |
| 3508 | heterogeneous nuclear ribonucleoprotein D-like |
| 3509 | tankyrase, TRF1-interacting ankyrin-related ADP-ribose polymerase 2 |
| 3510 | heterogeneous nuclear ribonucleoprotein K |
| 3511 | nucleoporin 98 kDa |
| 3512 | TAF9 RNA polymerase II, TATA box binding protein (TBP)-associated factor, 32 kDa |
| 3513 | Wolf-Hirschhom syndrome candidate 2 |
| 3514 | Wiskott-Aldrich syndrome protein interacting protein |
| 3515 | Rho GDP dissociation inhibitor (GDI) alpha |
| 3516 | nucleostemin |
| 3517 | single Ig IL-1R-related molecule |
| 3518 | chromosome 10 open reading frame 86 |
| 3519 | protease, serine, 23 |
| 3520 | — |

1. A method for investigating whether a test subject is affected by chronic fatigue syndrome/myalgic encephalomyelitis (CFS/ME), comprising providing a biological sample from the subject and determining the level in the sample of one or more biomarkers for CFS/ME, wherein the biomarkers comprise an expression product of at least one gene shown in Table 1.

2. A method according to claim 1, wherein the biomarkers comprise expression products of a plurality of genes shown in Table 1.

3. A method according to claim 2 wherein the biomarkers comprise expression products of 2, 3, 4, 5, 6, 7, 8, 9, 10, 15, 20 or more of the genes shown in Table 1.

4. A method according to claim 1 wherein the biomarkers comprise expression products of one or more of defensin α1, haemoglobin γ, CXCR4, tubulin beta 1 and HLA DRβ4 genes.

5. A method according to claim 4 wherein the biomarkers comprise expression products of two, three, four or five of defensin α1, haemoglobin γ, CXCR4, tubulin beta 1 and HLA DRβ4 genes.

6. A method according to claim 5 wherein the biomarkers comprise expression products of further hub genes of Table 1A.

7. A method according to claim 6 wherein the biomarkers further comprise expression products of one or more of thrombospondin 1, caspase 1 and/or IgG3.

8. A method according to claim 1 wherein the biomarkers comprise expression products of at least one gene from each of at least two of Tables 2, 3, 4 and 5.

9. A method according to claim 8 wherein said at least one gene from each said Table is a hub gene.

10. A method according to claim 4 wherein the biomarkers further comprise expression products of lactotransferrin (LTF) genes.

11. A method according to claim 10 wherein the biomarkers comprise expression products of defensin α4, integrin α2B, integrin β3, arginase 1, arginase 2, thrombospondin 1, membrane associated protein 17 (MAP 17), Charcot Leyden Crystal Protein (CLC) and/or chondroitin sulphate proteoglycan 2 (versican) genes.

12. A method according to claim 11 wherein the biomarkers comprise an expression product of haemoglobin γ (foetal haemoglobin).

13. A method according to claim 12 wherein the biomarkers comprise expression products of haemoglobin alpha 1, haemoglobin beta, haemoglobin delta, prostaglandin-endoperoxide synthase 1 and/or prostaglandin-endoperoxide synthase 2 genes.

14. A method according to claim 12 wherein the biomarkers comprise an expression product of serine/threonine kinase 17B (STK17B).

15. A method according to claim 14 wherein the biomarkers comprise expression products of caspase 1, dynamin 1-like, and/or phosphatidyl serine binding protein genes.

16. A method according to claim 15 wherein the biomarkers comprise an expression product of HLA-DRβ4.

17. A method according to claim 16 wherein the biomarkers comprise expression products of HLA-DQβ1 and/or immunoglobulin heavy chain γ3 genes.

18. A method according to claim 1 wherein the biomarkers comprise an expression product of one or more further genes from Table 6.

19. A method according to claim 18 wherein the biomarkers comprise an expression product of prostaglandin D2 synthase.

20. A method for classifying a subject affected by CFS/ME, the method comprising providing a biological sample from the subject and determining the level in the sample of one or more biomarkers for CFS/ME, wherein the biomarkers comprise expression products of at least one gene shown in one or more of Tables 2 to 5.

21. A method according to claim 20 wherein the biomarkers comprise expression products of a hub gene from one of Tables 2, 3, 4 or 5.

22. A method according to claim 21 wherein the biomarkers comprise expression products of hub genes from each of two, three or all of Tables 2, 3, 4 or 5.

23. A method according to claim 20 further comprising determining network genes from one, two, three or all of Tables 2, 3, 4 and 5.

24. A method according to claim 20 wherein the biomarkers comprise expression products of defensin α1, CXCR4 and lactotransferrin (LTF) genes.

25. A method according to claim 24 wherein the biomarkers comprise expression products of defensin α4, integrin α2B, integrin β3, arginase 1, arginase 2, thrombospondin 1, membrane associated protein 17 (MAP 17), Charcot Leyden Crystal Protein (CLC) and/or chondroitin sulphate proteoglycan 2 (versican) genes.

26. A method according to claim 20 wherein the biomarkers comprise an expression product of haemoglobin γ (foetal haemoglobin).

27. A method according to claim 26 wherein the biomarkers comprise expression products of haemoglobin alpha 1, haemoglobin beta, haemoglobin delta, prostaglandin-endoperoxide synthase 1 and/or prostaglandin-endoperoxide synthase 2 genes.

28. A method according to claim 20 wherein the biomarkers comprise an expression product of serine/threonine kinase 17B (STK17B).

29. A method according to claim 28 wherein the biomarkers comprise expression products of caspase 1, dynamin 1-like, and/or phosphatidyl serine binding protein genes.

30. A method according to claim 28 wherein the biomarkers comprise an expression product of HLA-DRβ4.

31. A method according to claim 30 wherein the biomarkers comprise expression products of HLA-DQβ1 and/or immunoglobulin heavy chain γ3 genes.

32. A method according to claim 31 further comprising classifying the subject as being affected by one or more of oxidative stress, excessive apoptosis, and immunological dysregulation (MHC I to II shift).

33. A method of determining whether an individual affected by CFS/ME is suitable for treatment with an anti-oxidant, minocycline or a corticosteroid, comprising determining whether the individual is affected by one or more of oxidative stress, excessive apoptosis, and immunological dysregulation by a method according to claim 24, and prescribing a suitable treatment depending on the outcome.

34. A method of creating a library of expression profiles for use in determining whether an individual is affected by CFS/ME, the method comprising
(a) providing biological samples from a plurality of individuals affected by CFS/ME, and determining the level in each sample of one or more biomarkers for CFS/ME to create a plurality of expression profiles from affected individuals;
(b) providing biological samples from a plurality of individuals not affected by CFS/ME, and determining the level in the sample of said one or more biomarkers for CFS/ME to create a plurality of expression profiles from unaffected individuals;
wherein the biomarkers comprise expression products of one or more genes shown in Table 1 and wherein said samples are optionally blood samples or comprise peripheral blood mononuclear cells isolated from a blood sample.

35. A method according to claim 34 comprising the step of retrievably storing each of the expression profiles on a computer data carrier, in order to create a database of expression profiles for both affected and unaffected individuals.

36. A method of determining the efficacy of a treatment for CFS/ME comprising the steps of:
(a) providing a biological sample from a subject affected by CFS/ME who has been subjected to said treatment,
(b) determining the level in said sample of one or more biomarkers for CFS/ME to create an expression profile for said subject, and
(c) comparing said expression profile with
i) a comparable expression profile obtained from said test subject before initiation of said treatment, and/or
ii) a comparable expression profile obtained from said test subject at an earlier stage of said treatment, and/or
iii) a comparable expression profile characteristic of a subject who is unaffected by CFS/ME,
wherein the one or more biomarkers for CFS/ME comprise expression products of one or more genes shown in Tables 1 to 5 and where said samples are optionally blood samples or comprise peripheral blood mononuclear cells isolated from a blood sample.

37. (canceled)

38. A method according to claim 34 wherein the expression level of each of the biomarkers is determined by contacting the sample with a binding agent capable of binding specifically to said expression product of the corresponding gene.

39. A method according to claim 38 wherein the expression product is a nucleic acid.

40. A method according to claim 39 wherein the binding agent is a nucleic acid probe or primer.

41. A method according to claim 39 wherein the expression product is mRNA, pre-mRNA or cDNA.

42. A method according to claim 41 wherein the expression level of the biomarker is determined by means of Northern blot, in situ hybridisation, RT-PCR, Southern blot or microarray assay.

43. A method according to claim 38 wherein the expression product is a polypeptide.

44. A method according to claim 43 wherein the binding agent is a ligand or receptor for said polypeptide, or an antibody or fragment thereof specific for said polypeptide

45. A method according to claim 43 wherein the expression level of the biomarker is determined by Western blot, ELISA or microarray assay.

46. A method according to claim 43 further comprising the step of determining the level, in the biological sample, or in a further biological sample from the subject, of the peptide QYNAD.

47. A method according to claim 46 wherein the further biological sample is a blood or serum sample.

48. A method according to claim 46 wherein the further biological sample is cerebrospinal fluid.

49. A kit for use in the method according to claim 1, the kit comprising a plurality of binding agents, each capable of binding specifically and individually to an expression product of one of the genes of Table 1, or the peptide QYNAD.

50. A kit according to claim 49 comprising binding agents specific for expression products of two or more genes of Tables 1, or at least one gene of Table 1 and the peptide QYNAD.

51. A kit according to claim 50 comprising binding agents capable of binding specifically and individually to expression products of 2, 3, 4, 5, 6, 7, 8, 9, 10, 15, 20 or more of the genes of Table 1 and optionally the peptide QYNAD.

52. A kit according to claim 51 wherein the binding agents are immobilised on one or more solid supports.

53. A kit according to claim 52 further comprising one or more binding agents capable of binding specifically to an expression product of a control gene which is substantially not differentially expressed between individuals affected and whose expression level is substantially unaffected by CFS/ME.

54. A kit according to claim 53 wherein the control gene is glyceraldehyde phosphate dehydrogenase (GAPDH), β-actin, or abl (ableson tyrosine kinase).

55. A kit according to claim 54 comprising binding agents for expression products of less than 1000 different genes, less than 500 different genes, less than 100, less than 50, less than 40, less than 30, less than 20, or less than 10 different genes.

56. A kit according to claim 55 wherein the expression product is a nucleic acid and the binding agent is a nucleic acid probe or primer.

57. A kit according to claim 56 wherein the expression product is mRNA, pre-mRNA or cDNA.

58. A kit according to claim 55 wherein the expression product is a polypeptide and the binding agent is a ligand or receptor for said polypeptide, or an antibody or fragment thereof specific for said polypeptide

59. An expression profile database, comprising a plurality of expression profiles of biomarkers for CFS/ME from affected and unaffected individuals, wherein the biomarkers comprise expression products of one or more genes shown in Table 1.

60. A method of treating CFS/ME in an individual suffering therefrom, comprising administering an effective amount of an anti-oxidant.

61. A method according to claim 60 further comprising administering therapeutically effective amounts of a corticosteroid and/or minocyline.

62-63. (canceled)

64. A method according to claim 60, wherein the anti-oxidant is coenzyme Q10 or an inhibitor of a cyclooxygenase (COX) enzyme such as celecoxib (4-[5-(4-methylphenyl)-3-(trifluoromethyl)-1H-pyrazol-1-yl] benzenesulfonamide).

65. A method of treating CFS/ME in an individual suffering therefrom, comprising administering an effective amount of minocycline.

66. A method according to claim 65 further comprising administering a therapeutically effective amount of a corticosteroid and/or an anti-oxidant.

67-68. (canceled)

69. A method of treating CFS/ME in an individual suffering therefrom, comprising administering an effective amount of a corticosteroid.

70. A method according to claim 69 further comprising administering a therapeutically effective amount of minocycline and/or an anti-oxidant.

71-72. (canceled)

73. A method according to claim 69, wherein the corticosteroid is hydrocortisone, dexamethasone or prednisone.

74. A pharmaceutical composition comprising therapeutically effective amounts of minocycline and a corticosteroid and/or an anti-oxidant, and a pharmaceutically acceptable carrier.

75-76. (canceled)
